(19) United States
(12) Patent Application Publication
(10) Pub. No.: US 2026/0066046 A1
(43) Pub. Date: Mar. 5, 2026

(54) METHODS FOR PROSTATE CANCER DETECTION AND TREATMENT

(71) Applicant: Liquid Biopsy Research LLC, Charlestown (KN)

(72) Inventors: Irvin Mark MODLIN, Woodbridge, CT (US); Mark KIDD, New Haven, CT (US); Ignat DROZDOV, Stratford Upon Avon (GB)

(21) Appl. No.: 19/182,880

(22) Filed: Apr. 18, 2025

Related U.S. Application Data
(63) Continuation of application No. 17/815,102, filed on Jul. 26, 2022, now abandoned.
Application No. 17/815,102 is a continuation of application No. 16/281,315, filed on Feb. 21, 2019, now Pat. No. 11,410,748.
(60) Provisional application No. 62/633,675, filed on Feb. 22, 2018.

(51) Int. Cl. G16B 25/10; C12Q 1/6886

(52) U.S. Cl. CPC G16B 25/10; C12Q 1/6886; C12Q 2600/106; C12Q 2600/112; C12Q 2600/158; G01N 2800/52

(57) ABSTRACT

The present invention is directed to methods for detecting a prostate cancer, methods for determining whether a prostate cancer is stable or progressive, low or high Gleason score, methods for differentiating benign prostate hyperplasia (BPH) from prostate cancer, methods for determining the completeness of surgery, and methods for evaluating the response to a prostate cancer therapy.

### CROSS-REFERENCE TO RELATED APPLICATIONS

This application is a continuation of U.S. patent application Ser. No. 17/815,102, filed Jul. 26, 2022, which is a continuation of U.S. patent application Ser. No. 16/281,315, filed Feb. 21, 2019, now U.S. Pat. No. 11,410,748, granted on Aug. 9, 2022, which claims priority to, and the benefit of, U.S. Provisional Application No. 62/633,675, filed Feb. 22, 2018. The contents of each of the aforementioned patent applications are incorporated herein by reference in their entireties.

### SEQUENCE LISTING

The Sequence Listing XML associated with this application is provided electronically in XML file format and is hereby incorporated by reference into the specification. The name of the XML file containing the Sequence Listing XML is “LBIO-005_C02US_SeqList.xml”. The XML filed is about 286,891 bytes in size, was created on Apr. 17, 2025, and is being submitted electronically via USPTO Patent Center.

### FIELD OF THE INVENTION

The present invention relates to prostate cancer detection.

### BACKGROUND OF THE INVENTION

Prostate cancer (PCA) is the fourth most commonly diagnosed cancer worldwide and the second most common cancer in men. Although the incidence and prevalence have been decreasing, ˜200,000 men will be diagnosed in the USA with PCA annually. Multiple factors including age and family history, genetic susceptibility and ethnicity all contribute to the high incidence of the disease. While 90% of PCA are diagnosed while they are localized (non-disseminated), the clinical behavior of tumors is highly variable and ranges from indolence that can be monitored through watchful waiting or active surveillance (e.g., biomarkers and 6 monthly digital rectal examination) to malignant evolution and androgen-resistant disease, metastatic dissemination and death.

Multiple risk stratification systems have been developed that combines clinical data and pathological information e.g., Gleason score. These, including the more recently developed next generation tools, are only ˜70% accurate for predicting outcome.

Molecular genetic information is increasingly being used to inform pathology and better subtype cancers. This information has been used as both prognostic tools as well as to stratify patients for different therapeutic interventions. Prostate cancers have been examined and mutations, DNA copy number alterations, rearrangements and gene fusions have all been identified. These may correlate with some pathological features. For example, low-Gleason tumors have few DNA copy number alterations while high grade tumors exhibit significant genome-wide copy number alterations. Somatic point mutations in contrast are relatively uncommon with the frequency of mutations ranging from 1% (IDH1) to 11% (SPOP). The most common abnormality is androgen-regulated fusions of ERG and other ETS family members (˜50% of tumors). However, fusion-bearing tumors do not have a significantly different prognosis to fusion-negative tumors after prostatectomy. Androgen receptor variant 7 (AR-V7) in contrast is implicated in the progression to castration resistance prostate cancer (CRPC) and is considered potentially useful as a treatment selection biomarker. Overall, however, there is an incomplete understanding of the molecular mechanisms underpinning PCA pathogenesis and an absence of molecular-based biomarkers that can be used to predict sensitivity to therapeutic agents. Consequently, the development of diagnostic methods that more accurately define disease status, identify sensitivity to therapy and can ultimately be used to better monitor disease progression, is critical.

Surveillance remains a cornerstone approach to monitor PCA and detect recurrence at an early stage. After potentially curative resection, monitoring can be undertaken through measurement of blood biomarkers and/or imaging like CT to detect asymptomatic metastatic disease earlier. The current biomarker used for monitoring is prostate specific antigen (PSA) (also gamma-seminoprotein or kallikrein-3). This glycoprotein enzyme is encoded by the KLK3 gene and is secreted by epithelial cells in the prostate. It, however, is not a unique indicator of prostate cancer, but may also detect prostatitis or benign prostatic hyperplasia (BPH). Use of PSA in isolation results in either unnecessary biopsies for men without cancer or an under diagnosis of men with significant disease. This is based on the low sensitivity (20-40%) and specificity (70-90%) ranges with a consequent positive predictive value of only 25-40%. The United States Preventive Services Task Force (USPSTF) does not recommend PSA use for prostate cancer. PSA, however, is included in clinical nomograms e.g., the UCSF-CAPRA score for prostate cancer risk, which has some utility in predicting disease free survival after surgery.

There remains a need for biomarker-based tools to accurately diagnose PCA.

### SUMMARY OF THE INVENTION

The present disclosure provides a method for detecting a prostate cancer in a subject in need thereof, the method comprising: determining the expression level of at least 38 biomarkers from a test sample from the subject by contacting the test sample with a plurality of agents specific to detect the expression of the at least 38 biomarkers, wherein the at least 38 biomarkers comprise AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, XPC, and a housekeeping gene; normalizing the expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC to the expression level of the housekeeping gene, thereby obtaining a normalized expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC; inputting each normalized expression level into an algorithm to generate a score; comparing the score with a first predetermined cutoff value; and identifying the presence of a prostate cancer in the subject when the score is equal to or greater than the first predetermined cutoff value or identifying the absence of a prostate cancer in the subject when the score is less than the first predetermined cutoff value.

The present disclosure provides a method for detecting a prostate cancer in a subject in need thereof, the method comprising: (a) determining the expression level of at least 38 biomarkers from a test sample from the subject by contacting the test sample with a plurality of agents specific to detect the expression of the at least 38 biomarkers, wherein the at least 38 biomarkers comprise AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, XPC, and a housekeeping gene; (b) normalizing the expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC to the expression level of the housekeeping gene, thereby obtaining a normalized expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC; (c) inputting each normalized expression level into an algorithm to generate a score; (d) comparing the score with a first predetermined cutoff value; and (e) producing a report, wherein the report identifies the presence of a prostate cancer in the subject when the score is equal to or greater than the first predetermined cutoff value or identifies the absence of a prostate cancer in the subject when the score is less than the first predetermined cutoff value.

The present disclosure also provides a method for determining whether a prostate cancer in a subject is stable or progressive, the method comprising: determining the expression level of at least 38 biomarkers from a test sample from the subject by contacting the test sample with a plurality of agents specific to detect the expression of the at least 38 biomarkers, wherein the at least 38 biomarkers comprise AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, XPC, and a housekeeping gene; normalizing the expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC to the expression level of the housekeeping gene, thereby obtaining a normalized expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC; inputting each normalized expression level into an algorithm to generate a score; comparing the score with a first predetermined cutoff value; and identifying that the prostate cancer is progressive when the score is equal to or greater than the first predetermined cutoff value or identifying that the prostate cancer is stable when the score is less than the first predetermined cutoff value.

The present disclosure provides a method for determining whether a prostate cancer in a subject is stable or progressive, the method comprising: (a) determining the expression level of at least 38 biomarkers from a test sample from the subject by contacting the test sample with a plurality of agents specific to detect the expression of the at least 38 biomarkers, wherein the at least 38 biomarkers comprise AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, XPC, and a housekeeping gene; (b) normalizing the expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC to the expression level of the housekeeping gene, thereby obtaining a normalized expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC; (c) inputting each normalized expression level into an algorithm to generate a score; (d) comparing the score with a first predetermined cutoff value; and (e) producing a report, wherein the report identifies that the prostate cancer is progressive when the score is equal to or greater than the first predetermined cutoff value or identifies that the prostate cancer is stable when the score is less than the first predetermined cutoff value.

The present disclosure also provides a method for determining whether a prostate cancer in a subject is low or high grade, the method comprising: determining the expression level of at least 38 biomarkers from a test sample from the subject by contacting the test sample with a plurality of agents specific to detect the expression of the at least 38 biomarkers, wherein the at least 38 biomarkers comprise AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, XPC, and a housekeeping gene; normalizing the expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC to the expression level of the housekeeping gene, thereby obtaining a normalized expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC; inputting each normalized expression level into an algorithm to generate a score; comparing the score with a first predetermined cutoff value; and identifying that the prostate cancer is high grade when the score is equal to or greater than the first predetermined cutoff value or identifying that the prostate cancer is low grade when the score is less than the first predetermined cutoff value.

The present disclosure provides a method for determining whether a prostate cancer in a subject is low or high grade, the method comprising: determining the expression level of at least 38 biomarkers from a test sample from the subject by contacting the test sample with a plurality of agents specific to detect the expression of the at least 38 biomarkers, wherein the at least 38 biomarkers comprise AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, XPC, and a housekeeping gene; normalizing the expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC to the expression level of the housekeeping gene, thereby obtaining a normalized expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC; inputting each normalized expression level into an algorithm to generate a score; comparing the score with a first predetermined cutoff value; and producing a report, wherein the report identifies that the prostate cancer is high grade when the score is equal to or greater than the first predetermined cutoff value or identifies that the prostate cancer is low grade when the score is less than the first predetermined cutoff value.

The present disclosure also provides a method for determining whether a prostate cancer in a subject is a low Gleason score (≤6) prostate cancer or a high Gleason score (≥7) prostate cancer, the method comprising: (a) determining the expression level of at least 38 biomarkers from a test sample from the subject by contacting the test sample with a plurality of agents specific to detect the expression of the at least 38 biomarkers, wherein the at least 38 biomarkers comprise AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, XPC, and a housekeeping gene; (b) normalizing the expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC to the expression level of the housekeeping gene, thereby obtaining a normalized expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC; (c) inputting each normalized expression level into an algorithm to generate a score; (d) comparing the score with a first predetermined cutoff value; and (e) identifying that the prostate cancer is a high Gleason score prostate cancer when the score is equal to or greater than the first predetermined cutoff value or identifying that the prostate cancer is a low Gleason score prostate cancer when the score is less than the first predetermined cutoff value.

The present disclosure provides a method for determining whether a prostate cancer in a subject is a low Gleason score (≤6) prostate cancer or a high Gleason score (≥7) prostate cancer, the method comprising: (a) determining the expression level of at least 38 biomarkers from a test sample from the subject by contacting the test sample with a plurality of agents specific to detect the expression of the at least 38 biomarkers, wherein the at least 38 biomarkers comprise AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, XPC, and a housekeeping gene; (b) normalizing the expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC to the expression level of the housekeeping gene, thereby obtaining a normalized expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC; (c) inputting each normalized expression level into an algorithm to generate a score; (d) comparing the score with a first predetermined cutoff value; and (e) producing a report, wherein the report identifies that the prostate cancer is a high Gleason score prostate cancer when the score is equal to or greater than the first predetermined cutoff value or identifies that the prostate cancer is a low Gleason score prostate cancer when the score is less than the first predetermined cutoff value.

The present disclosure also provides a method for determining the completeness of surgery in a subject having a prostate cancer, the method comprising: determining the expression level of at least 38 biomarkers from a test sample from the subject after the surgery by contacting the test sample with a plurality of agents specific to detect the expression of the at least 38 biomarkers, wherein the 38 biomarkers comprise AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_J, TMPRSS2_2, TRIM29, UNC45A, XPC, and a housekeeping gene; normalizing the expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC to the expression level of the housekeeping gene, thereby obtaining a normalized expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC; inputting each normalized expression level into an algorithm to generate a score; comparing the score with a first predetermined cutoff value; and identifying that the prostate cancer is not completely removed when the score is equal to or greater than the first predetermined cutoff value or identifying that the prostate cancer is completely removed when the score is less than the first predetermined cutoff value.

The present disclosure provides a method for determining the completeness of surgery in a subject having a prostate cancer, the method comprising: (a) determining the expression level of at least 38 biomarkers from a test sample from the subject after the surgery by contacting the test sample with a plurality of agents specific to detect the expression of the at least 38 biomarkers, wherein the 38 biomarkers comprise AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, XPC, and a housekeeping gene; (b) normalizing the expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC to the expression level of the housekeeping gene, thereby obtaining a normalized expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC; (c) inputting each normalized expression level into an algorithm to generate a score; (d) comparing the score with a first predetermined cutoff value; and (e) producing a report, wherein the report identifies that the prostate cancer is not completely removed when the score is equal to or greater than the first predetermined cutoff value or identifies that the prostate cancer is completely removed when the score is less than the first predetermined cutoff value.

The present disclosure also provides a method for differentiating benign prostate hyperplasia from a prostate cancer in a subject having an enlarged prostate, the method comprising: determining the expression level of at least 38 biomarkers from a test sample from the subject by contacting the test sample with a plurality of agents specific to detect the expression of the at least 38 biomarkers, wherein the at least 38 biomarkers comprise AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, XPC, and a housekeeping gene; normalizing the expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC to the expression level of the housekeeping gene, thereby obtaining a normalized expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC; inputting each normalized expression level into an algorithm to generate a score; comparing the score with a first predetermined cutoff value; and identifying the presence of a prostate cancer in the subject when the score is equal to or greater than the first predetermined cutoff value or identifying the presence of benign prostate hyperplasia in the subject when the score is less than the first predetermined cutoff value.

The present disclosure provides a method for differentiating benign prostate hyperplasia from a prostate cancer in a subject having an enlarged prostate, the method comprising: (a) determining the expression level of at least 38 biomarkers from a test sample from the subject by contacting the test sample with a plurality of agents specific to detect the expression of the at least 38 biomarkers, wherein the at least 38 biomarkers comprise AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, XPC, and a housekeeping gene; (b) normalizing the expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC to the expression level of the housekeeping gene, thereby obtaining a normalized expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC; (c) inputting each normalized expression level into an algorithm to generate a score; (d) comparing the score with a first predetermined cutoff value; and (e) producing a report, wherein the report identifies the presence of a prostate cancer in the subject when the score is equal to or greater than the first predetermined cutoff value or identifies the presence of benign prostate hyperplasia in the subject when the score is less than the first predetermined cutoff value.

The present disclosure provides a method for evaluating the response of a subject having a prostate cancer to a first therapy, the method comprising: (1) at a first time point: (a) determining the expression level of at least 38 biomarkers from a test sample from the subject by contacting the test sample with a plurality of agents specific to detect the expression of the at least 38 biomarkers, wherein the at least 38 biomarkers comprise AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, XPC, and a housekeeping gene; (b) normalizing the expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC to the expression level of the housekeeping gene, thereby obtaining a normalized expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC; (c) inputting each normalized expression level into an algorithm to generate a first score; (2) at a second time point, wherein the second time point is after the first time point and after the administration of the first therapy to the subject: (d) determining the expression level of the at least 38 biomarkers from a test sample from the subject by contacting the test sample with a plurality of agents specific to detect the expression of the at least 38 biomarkers; (e) normalizing the expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC to the expression level of the housekeeping gene, thereby obtaining a normalized expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC; (f) inputting each normalized expression level into an algorithm to generate a second score; (3) comparing the first score with the second score; and (4) identifying the subject as responsive to the first therapy when the second score is significantly decreased as compared to the first score or identifying the subject as not responsive to the first therapy when the second score is not significantly decreased as compared to the first score.

The preceding method can further comprise continuing to administer the first therapy to the subject when the second score is significantly decreased as compared to the first score. The preceding method can further comprise discontinuing administration of the first therapy to the subject when the second score is not significantly decreased as compared to the first score. The preceding method can further comprise administering a second therapy to the subject when the second score is not significantly decreased as compared to the first score.

The present disclosure provides a method for evaluating the response of a subject having a prostate cancer to a first therapy, the method comprising: (1) at a first time point: (a) determining the expression level of at least 38 biomarkers from a test sample from the subject by contacting the test sample with a plurality of agents specific to detect the expression of the at least 38 biomarkers, wherein the at least 38 biomarkers comprise AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, XPC, and a housekeeping gene; (b) normalizing the expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC to the expression level of the housekeeping gene, thereby obtaining a normalized expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC; (c) inputting each normalized expression level into an algorithm to generate a first score; (2) at a second time point, wherein the second time point is after the first time point and after the administration of the therapy to the subject: (d) determining the expression level of the at least 38 biomarkers from a test sample from the subject by contacting the test sample with a plurality of agents specific to detect the expression of the at least 38 biomarkers; (e) normalizing the expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC to the expression level of the housekeeping gene, thereby obtaining a normalized expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC; (f) inputting each normalized expression level into an algorithm to generate a second score; (3) comparing the first score with the second score; and (4) producing a report, wherein the report identifies that the subject is responsive to the first therapy when the second score is significantly decreased as compared to the first score or identifies that the subject is not responsive to the first therapy when the second score is not significantly decreased as compared to the first score.

The preceding method can further comprise continuing to administer the first therapy to the subject when the second score is significantly decreased as compared to the first score. The preceding method can further comprise discontinuing administration of the first therapy to the subject when the second score is not significantly decreased as compared to the first score. The preceding method can further comprise administering a second therapy to the subject when the second score is not significantly decreased as compared to the first score.

The present disclosure also provides a method for treating a prostate cancer in a subject in need thereof, the method comprising: determining the expression level of at least 38 biomarkers from a test sample from the subject by contacting the test sample with a plurality of agents specific to detect the expression of the at least 38 biomarkers, wherein the at least 38 biomarkers comprise AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, XPC, and a housekeeping gene; normalizing the expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC to the expression level of the housekeeping gene, thereby obtaining a normalized expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC; inputting each normalized expression level into an algorithm to generate a score; comparing the score with a first predetermined cutoff value; and administering at least a first therapy to the subject when the score is equal to or greater than the first predetermined cutoff value or identifies the absence of a prostate cancer in the subject when the score is less than the first predetermined cutoff value.

In the methods of the present disclosure, the housekeeping gene can be selected from the group consisting of ALG9, SEPN, YWHAQ, VPS37A, PRRC2B, DOPEY2, NDUFBI1, ND4, MRPL19, PSMC4, SF3A1, PUM1, ACTB, GAPD, GUSB, RPLPO, TFRC, MORF4L1, 18S, PPIA, PGK1, RPL13A, B2M, YWHAZ, SDHA, HPRT1, TOX4 and TPT1. The housekeeping gene can be TOX4.

In the methods of the present disclosure, a predetermined cutoff value can be at least 33% on a scale of 0-100%, or at least 50% on a scale of 0-100%. A first predetermined cutoff value can be at least 33% on a scale of 0-100%, or at least 50% on a scale of 0-100%.

The methods of the present disclosure can further comprise administering a therapy to a subject. The methods of the present disclosure can further comprise administering a first therapy a subject. The methods of the present disclosure can further comprise administering a second therapy to a subject.

In the methods of the present disclosure, a therapy can comprise active surveillance, radiation therapy, surgery, cryotherapy, hormone therapy, chemotherapy, vaccine treatment, bone-directed treatment, or any combination thereof. A first therapy can comprise active surveillance, radiation therapy, surgery, cryotherapy, hormone therapy, chemotherapy, vaccine treatment, bone-directed treatment, or any combination thereof. A second therapy can comprise active surveillance, radiation therapy, surgery, cryotherapy, hormone therapy, chemotherapy, vaccine treatment, bone-directed treatment, or any combination thereof.

In some aspects, hormone therapy can comprise androgen suppression therapy. In some aspects, chemotherapy can comprise docetaxel, cabazitaxel, mitoxantrone, estramustine, or a combination thereof. In some aspects, a vaccine treatment can comprise Sipuleucel-T. In some aspects, a bone-directed treatment can comprise a bisphosphonate, denosumab, a corticosteroid, or a combination thereof.

In the methods of the present disclosure, an algorithm can be XGB, RF, glmnet, cforest, CART, treebag, knn, nnet, SVM-radial, SVM-linear, NB, or mlp. An algorithm can be XGB.

In the methods of the present disclosure, a first time point can be either prior to or after the administration of a therapy to the subject. A first time point can be either prior to or after the administration of a first therapy to the subject.

In the methods of the present disclosure, a second score is significantly decreased as compared to the first score when the second score is at least 25% less than the first score.

The methods of the present disclosure can have a sensitivity of at least 92%. The methods of the present disclosure can have a specificity of at least 95%.

In the methods of the present disclosure, at least one of the at least 38 biomarkers can be RNA, cDNA, or protein.

In the methods of the present disclosure, when the biomarker is RNA, the RNA can be reverse transcribed to produce cDNA, and the produced cDNA expression level can be detected.

In the methods of the present disclosure, the expression level of a biomarker can be detected by forming a complex between the biomarker and a labeled probe or primer.

In the methods of the present disclosure, when the biomarker is protein, the protein can be detected by forming a complex between the protein and a labeled antibody. The label can be a fluorescent label.

In the methods of the present disclosure, when the biomarker is RNA or cDNA, the RNA or cDNA can be detected by forming a complex between the RNA or cDNA and a labeled nucleic acid probe or primer. For example, a label can be a fluorescent label. A complex between the RNA or cDNA and the labeled nucleic acid probe or primer can be a hybridization complex.

In the methods of the present disclosure, a predetermined cutoff value can be derived from a plurality of reference samples obtained from subjects not having or not diagnosed with a neoplastic disease. The neoplastic disease can be prostate cancer.

In the methods of the present disclosure, a test sample can be blood, serum, plasma, or neoplastic tissue. A reference sample can be blood, serum, plasma, or non-neoplastic tissue.

In the methods of the present disclosure, a subject can have at least one prostate cancer symptom. In the methods of the present disclosure, a subject can have a predisposition or familial history for developing a prostate cancer.

In the methods of the present disclosure, a subject can have been previously diagnosed with a prostate cancer and is tested for prostate cancer recurrence.

In the methods of the present disclosure, a subject can be human.

Any of the above aspects can be combined with any other aspect.

Unless otherwise defined, all technical and scientific terms used herein have the same meaning as commonly understood by one of ordinary skill in the art to which this disclosure belongs. In the Specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs. All patents and publications cited in this specification are incorporated herein by reference in their entireties.

### BRIEF DESCRIPTION OF THE DRAWINGS

The patent or application file contains at least one drawing executed in color. Copies of this patent or patent application publication with color drawings will be provided by the Office upon request and payment of the necessary fee.

The above and further features will be more clearly appreciated from the following detailed description when taken in conjunction with the accompanying drawings.

FIGS. 1A-1B are graphs showing visualisation of 30 putative marker genes identified by the Random Forest algorithm in the derivation cohort of n=595 tissue samples. (FIG. 1A) Expression in E-GEOD-46691. (FIG. 1B) Expression in E-GEOD-46602.

FIG. 2 is a scatter plot of average predictive importance vs. Kruskal Wallis p-values. Vertical and horizontal lines represent p-value of 0.05 and median Predictive Importance value respectively.

FIG. 3 is a graph showing hierarchical clustering of gene expression identified that PCA tumor tissue and control tissue were separately clustered. Gene expression was significantly higher in the PCA tissue.

FIG. 4 is a graph showing clustering of normalized gene expression separates prostate-derived cell lines into their tissue of origin (i.e., normal, localized, metastatic).

FIG. 5 is a graph showing gene expression in the control (n=201), BPH (prostate hyperplasia, n=26) and prostate cancer cases (n=125). Identified expression levels were significantly (p<0.0001) elevated in cases (63±19%) versus benign prostatic hyperplasia (BPH: 17±13%) and controls (8±9%).

FIG. 6 is a graph showing receiver operator curve analysis. The AUROC was 0.97 and the Youden J index was 0.94. The Z-statistic was highly significant (38.9; p<0.0001).

FIG. 7 is a graph showing the metrics of the ProstaTest for determining PCA ranged 92-99%.

FIG. 8 is a Probit risk assessment plot that identifies a ProstaTest score >30 was 60% accurate for predicting prostate cancer in a blood sample. This was increased to >80% at a test scores >34. Dotted lines represent the 95% confidence interval.

FIG. 9 is a graph showing receiver operator curve analysis comparing the ProstaTest with measurements of PSA for predicting high grade (>Gleason 7) compared to low grade (Gleason 5+6) tumors. The AUROC was 0.98 and the Youden J index was 0.87 for the ProstaTest. For PSA, the AUROC was 0.64 and the Youden J index was 0.42. The ProstaTest was significantly better than PSA for predicting grade (Z-statistic: 3.05, p=0.002).

FIG. 10 is a graph showing the effect of surgery on the ProstaTest. Levels prior to surgery are elevated (80±18%). Surgery reduced levels to 22±7% (p<0.0001), not different to control levels.

FIG. 11 is a graph showing the effect of treatment on the ProstaTest. Levels prior to treatment are elevated (74±18%). In those who responded to hormone therapy, levels were reduced to 18±7% (p<0.0001). In those requiring chemotherapy, levels were reduced to 27±10% (p<0.001).

### DETAILED DESCRIPTION OF THE INVENTION

The details of the invention are set forth in the accompanying description below.

Described herein are methods to quantitate (score) the circulating prostate cancer molecular signature with high sensitivity and specificity for purposes including, but not limited to, detecting a prostate cancer, determining whether a prostate cancer is stable or progressive, differentiating benign prostate hyperplasia (BPH) from prostate cancer, determining the completeness of surgery, and evaluating the response to a prostate cancer therapy. Specifically, the present invention is based on the discovery that the expression levels of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC, normalized by the expression level of a housekeeping gene, are elevated in subjects having prostate cancers as compared to healthy subjects or subjects with BPH.

Symptoms of prostate cancers include problems urinating, blood in the urine or semen, trouble getting an erection, pain in the hips, back (spine), chest (ribs), or other areas from cancer that has spread to bones, weakness or numbness in the legs or feet, or even loss of bladder or bowel control from cancer pressing on the spinal cord.

As described in the examples, measurements of circulating prostate cancer transcripts—the ProstaTest— diagnoses prostate cancer and decreases in the ProstaTest score in blood correlates with the efficacy of therapeutic interventions such as surgery and chemotherapy. A targeted gene expression profile of prostate cancer RNA can be isolated from the peripheral blood of patients. The expression profile is evaluated in an algorithm and converted to an output (score). It can diagnose and identify active disease and provide an assessment of treatment responses in conjunction with standard clinical assessment and imaging.

Accordingly, the present disclosure provides a method for detecting a prostate cancer in a subject in need thereof, the method comprising: (a) determining the expression level of at least 38 biomarkers from a test sample from the subject by contacting the test sample with a plurality of agents specific to detect the expression of the at least 38 biomarkers, wherein the at least 38 biomarkers comprise AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, XPC, and a housekeeping gene; (b) normalizing the expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC to the expression level of the housekeeping gene, thereby obtaining a normalized expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC; (c) inputting each normalized expression level into an algorithm to generate a score; (d) comparing the score with a first predetermined cutoff value; and (e) identifying the presence of a prostate cancer in the subject when the score is equal to or greater than the first predetermined cutoff value or identifying the absence of a prostate cancer in the subject when the score is less than the first predetermined cutoff value.

In some aspects of the preceding method, step (e) can comprise producing a report, wherein the report identifies the presence of a prostate cancer in the subject when the score is equal to or greater than the first predetermined cutoff value or identifies the absence of a prostate cancer in the subject when the score is less than the first predetermined cutoff value.

In some aspects of the preceding method, the first predetermined cutoff value can be 33% on a scale of 0-100%.

In some aspects, the preceding method can further comprise administering to the subject a first therapy. The preceding method can further comprise administering to the subject a first therapy when the score is equal to or greater than the predetermined cutoff value.

The present disclosure also provides a method for determining whether a prostate cancer in a subject is stable or progressive, the method comprising: (a) determining the expression level of at least 38 biomarkers from a test sample from the subject by contacting the test sample with a plurality of agents specific to detect the expression of the at least 38 biomarkers, wherein the at least 38 biomarkers comprise AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, XPC, and a housekeeping gene; (b) normalizing the expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC to the expression level of the housekeeping gene, thereby obtaining a normalized expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC; (c) inputting each normalized expression level into an algorithm to generate a score; (d) comparing the score with a first predetermined cutoff value; and (e) identifying that the prostate cancer is progressive when the score is equal to or greater than the first predetermined cutoff value or identifying that the prostate cancer is stable when the score is less than the first predetermined cutoff value.

In some aspects of the preceding method, step (e) can comprise producing a report, wherein the report identifies that the prostate cancer is progressive when the score is equal to or greater than the first predetermined cutoff value or identifies that the prostate cancer is stable when the score is less than the first predetermined cutoff value.

In some aspects of the preceding method, the first predetermined cutoff value can be 50% on a scale of 0-100%.

In some aspects, the preceding method can further comprise administering to the subject a first therapy. The preceding method can further comprise administering to the subject a first therapy when the score is equal to or greater than the predetermined cutoff value.

The present disclosure also provides a method for determining whether a prostate cancer in a subject is low or high grade, the method comprising: (a) determining the expression level of at least 38 biomarkers from a test sample from the subject by contacting the test sample with a plurality of agents specific to detect the expression of the at least 38 biomarkers, wherein the at least 38 biomarkers comprise AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, XPC, and a housekeeping gene; (b) normalizing the expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC to the expression level of the housekeeping gene, thereby obtaining a normalized expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC; (c) inputting each normalized expression level into an algorithm to generate a score; (d) comparing the score with a first predetermined cutoff value; and (e) identifying that the prostate cancer is high grade when the score is equal to or greater than the first predetermined cutoff value or identifying that the prostate cancer is low grade when the score is less than the first predetermined cutoff value.

In some aspects of the preceding method, step (e) can comprise producing a report, wherein the report identifies that the prostate cancer is high grade when the score is equal to or greater than the first predetermined cutoff value or identifies that the prostate cancer is low grade when the score is less than the first predetermined cutoff value.

In some aspects of the preceding method, the first predetermined cutoff value can be 50% on a scale of 0-100%.

In some aspects, the preceding method can further comprise administering to the subject a first therapy. The preceding method can further comprise administering to the subject a first therapy when the score is equal to or greater than the predetermined cutoff value.

In some aspects, a low grade prostate cancer is a prostate cancer with a Gleason score that is less than or equal to 6. In some aspects, a high grade prostate cancer is a prostate cancer with a Gleason score that is greater than or equal to 7.

The Gleason Grading System is commonly used in the art as a parameter of prognosis, often used in combination with other prognostic factors or tests, for prostate cancer. Prostate biopsy samples are examined, for example, by microscope, and a Gleason score is determined by a pathologist, based on the architectural pattern of the prostate tumor. The Gleason score is based upon the degree of loss of the normal glandular tissue architecture (i.e. shape, size and differentiation of the glands). The sample is assigned a grade to the most common tumor pattern, and a second grade to the next most common tumor pattern. There may be a primary or most common pattern and then a secondary or second most common pattern which can be identified; alternatively, there may be only a single grade. Gleason patterns are associated with the following features: Pattern 1—The cancerous prostate closely resembles normal prostate tissue. The glands are small, well-formed, and closely packed; Pattern 2—The tissue still has well-formed glands, but they are larger and have more tissue between them; Pattern 3—The tissue still has recognizable glands, but the cells are darker. At high magnification, some of these cells have left the glands and are beginning to invade the surrounding tissue; Pattern 4—The tissue has few recognizable glands. Many cells are invading the surrounding tissue; Pattern 5—The tissue does not have recognizable glands. There are often just sheets of cells throughout the surrounding tissue. The two grades are added together to get a Gleason Score, also known as a Gleason sum.

The present disclosure also provides a method for determining whether a prostate cancer in a subject is a low Gleason score (≤6) prostate cancer or a high Gleason score (≥7) prostate cancer, the method comprising: (a) determining the expression level of at least 38 biomarkers from a test sample from the subject by contacting the test sample with a plurality of agents specific to detect the expression of the at least 38 biomarkers, wherein the at least 38 biomarkers comprise AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, XPC, and a housekeeping gene; (b) normalizing the expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC to the expression level of the housekeeping gene, thereby obtaining a normalized expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC; (c) inputting each normalized expression level into an algorithm to generate a score; (d) comparing the score with a second predetermined cutoff value; and (e) identifying that the prostate cancer is a high Gleason score prostate cancer when the score is equal to or greater than the first predetermined cutoff value or identifying that the prostate cancer is a low Gleason score prostate when the score is less than the first predetermined cutoff value.

In some aspects of the preceding method, step (e) can comprise producing a report, wherein the report identifies that the prostate cancer is a high Gleason score prostate cancer when the score is equal to or greater than the first predetermined cutoff value or identifies that the prostate cancer is a low Gleason score prostate cancer when the score is less than the second predetermined cutoff value,

In some aspects of the preceding method, the first predetermined cutoff value can be 50% on a scale of 0-100%.

In some aspects, the preceding method can further comprise administering to the subject a first therapy. The preceding method can further comprise administering to the subject a first therapy when the score is equal to or greater than the predetermined cutoff value.

The present disclosure also provides a method for determining the completeness of surgery in a subject having a prostate cancer, the method comprising: (a) determining the expression level of at least 38 biomarkers from a test sample from the subject after the surgery by contacting the test sample with a plurality of agents specific to detect the expression of the at least 38 biomarkers, wherein the 38 biomarkers comprise AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, XPC, and a housekeeping gene; (b) normalizing the expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC to the expression level of the housekeeping gene, thereby obtaining a normalized expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC; (c) inputting each normalized expression level into an algorithm to generate a score; (d) comparing the score with a first predetermined cutoff value; and (e) identifying that the prostate cancer is not completely removed when the score is equal to or greater than the first predetermined cutoff value or identifying that the prostate cancer is completely removed when the score is less than the first predetermined cutoff value.

In some aspects of the preceding method, step (e) can comprise producing a report, wherein the report identifies that the prostate cancer is not completely removed when the score is equal to or greater than the first predetermined cutoff value or identifies that the prostate cancer is completely removed when the score is less than the first predetermined cutoff value.

In some aspects of the preceding method, the first predetermined cutoff value can be 33% on a scale of 0-100%.

In some aspects, the preceding method can further comprise administering to the subject a first therapy. The preceding method can further comprise administering to the subject a first therapy when the score is equal to or greater than the predetermined cutoff value.

The present disclosure also provides a method for differentiating benign prostate hyperplasia from a prostate cancer in a subject having an enlarged prostate, the method comprising: (a) determining the expression level of at least 38 biomarkers from a test sample from the subject by contacting the test sample with a plurality of agents specific to detect the expression of the at least 38 biomarkers, wherein the at least 38 biomarkers comprise AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, XPC, and a housekeeping gene; (b) normalizing the expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC to the expression level of the housekeeping gene, thereby obtaining a normalized expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC; (c) inputting each normalized expression level into an algorithm to generate a score; (d) comparing the score with a first predetermined cutoff value; and (e) identifying the presence of a prostate cancer in the subject when the score is equal to or greater than the first predetermined cutoff value or identifying the presence of benign prostate hyperplasia in the subject when the score is less than the first predetermined cutoff value.

In some aspects of the preceding method, step (e) can comprise producing a report, wherein the report identifies the presence of a prostate cancer in the subject when the score is equal to or greater than the first predetermined cutoff value or identifies the presence of benign prostate hyperplasia in the subject when the score is less than the first predetermined cutoff value,

In some aspects of the preceding method, the first predetermined cutoff value can be 33% on a scale of 0-100%.

In some aspects, the preceding method can further comprise administering to the subject a first therapy. The preceding method can further comprise administering to the subject a first therapy when the score is equal to or greater than the predetermined cutoff value.

The present disclosure provides a method for treating a prostate cancer in a subject in need thereof, the method comprising: (a) determining the expression level of at least 38 biomarkers from a test sample from the subject by contacting the test sample with a plurality of agents specific to detect the expression of the at least 38 biomarkers, wherein the at least 38 biomarkers comprise AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, XPC, and a housekeeping gene; (b) normalizing the expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC to the expression level of the housekeeping gene, thereby obtaining a normalized expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC; (c) inputting each normalized expression level into an algorithm to generate a score; (d) comparing the score with a first predetermined cutoff value; and (e) administering at least a first therapy to the subject when the score is equal to or greater than the first predetermined cutoff value or identifies the absence of a prostate cancer in the subject when the score is less than the first predetermined cutoff value.

The present disclosure also provides a method for evaluating the response of a subject having a prostate cancer to a first therapy, the method comprising: (1) at a first time point: (a) determining the expression level of at least 38 biomarkers from a test sample from the subject by contacting the test sample with a plurality of agents specific to detect the expression of the at least 38 biomarkers, wherein the at least 38 biomarkers comprise AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, XPC, and a housekeeping gene; (b) normalizing the expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC to the expression level of the housekeeping gene, thereby obtaining a normalized expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC; (c) inputting each normalized expression level into an algorithm to generate a first score; (2) at a second time point, wherein the second time point is after the first time point and after the administration of the first therapy to the subject: (d) determining the expression level of the at least 38 biomarkers from a test sample from the subject by contacting the test sample with a plurality of agents specific to detect the expression of the at least 38 biomarkers; (e) normalizing the expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC to the expression level of the housekeeping gene, thereby obtaining a normalized expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC; (f) inputting each normalized expression level into an algorithm to generate a second score; (3) comparing the first score with the second score; and (4) identifying the subject as responsive to the first therapy when the second score is significantly decreased as compared to the first score or identifying the subject as not responsive to the first therapy when the second score is not significantly decreased as compared to the first score.

In some aspects of the preceding method, step (4) can comprise producing a report, wherein the report identifies that the subject is responsive to the first therapy when the second score is significantly decreased as compared to the first score or identifies that the subject is not responsive to the first therapy when the second score is not significantly decreased as compared to the first score.

In some aspects, the preceding method can further comprise continuing to administer the first therapy to the subject when the second score is significantly decreased as compared to the first score. The preceding method can further comprise discontinuing administration of the first therapy to the subject when the second score is not significantly decreased as compared to the first score. The preceding method can further comprise administering a second therapy to the subject when the second score is not significantly decreased as compared to the first score.

In some aspects of the preceding method, the second score is significantly decreased as compared to the first score when the second score is at least about 10% less than the first score, or at least about 20% less than the first score, or at least about 25% less than the first score, or at least about 30% less than the first score, at least about 40% less than the first score, at least about 50% less than the first score, or at least about 60% less than the first score, or at least about 70% less than the first score, or at least about 75% less than the first score, or at least about 80% less than the first score, or at least about 90% less than the first score, or at least about 95% less than the first score or at least about 99% less than the first score. In some aspects, when the second score is not significantly decreased as compared to the first score, the subject is considered to be not responsive to the therapy.

In some aspects of the preceding method, a first time point can be prior to the administration of a first therapy to the subject. A first time point can be after the administration of a first therapy to the subject.

In some aspects of the methods of the present disclosure, housekeeping genes include, but are not limited to, ALG9, SEPN, YWHAQ, VPS37A, PRRC2B, DOPEY2, NDUFBI1, ND4, MRPL19, PSMC4, SF3A1, PUM1, ACTB, GAPD, GUSB, RPLPO, TFRC, MORF4L1, 18S, PPIA, PGK1, RPL13A, B2M, YWHAZ, SDHA, HPRT1, TOX4 and TPT1. In some aspects, the housekeeping gene is TOX4.

In some aspects of the methods of the present disclosure, a predetermined cutoff value can be about 33% on a scale of 0-100%. In some aspects of the methods of the present disclosure, a predetermined cutoff value can be about 50% on a scale of 0-100%. A predetermined cutoff value can be about 60% on a scale of 0-100%. A predetermine cutoff value can be about 10%, or about 20%, or about 30%, or about 40%, or about 70%, or about 80%, or about 90% on a scale of 0-100%.

The methods of the present disclosure can have a sensitivity of at least about 50%, or at least about 60%, or at least about 70%, or at least about 75%, or at least about 80%, or at least about 85%, or at least about 90%, or at least about 95%, or at least about 99%. The methods of the present disclosure can have a sensitivity of greater than about 50%, or greater than about 60%, or greater than about 70%, or greater than about 75%, or greater than about 80%, or greater than about 85%, or greater than about 90%, or greater than about 95%, or greater than about 99%.

The methods of the present disclosure can have a specificity of at least about 50%, or at least about 60%, or at least about 70%, or at least about 75%, or at least about 80%, or at least about 85%, or at least about 90%, or at least about 95%, or at least about 99%. The methods of the present disclosure can have a specificity of greater than about 50%, or greater than about 60%, or greater than about 70%, or greater than about 75%, or greater than about 80%, or greater than about 85%, or greater than about 90%, or greater than about 95%, or greater than about 99%.

The methods of the present disclosure can have an accuracy of at least about 50%, or at least about 60%, or at least about 70%, or at least about 75%, or at least about 80%, or at least about 85%, or at least about 90%, or at least about 95%, or at least about 99%. The methods of the present disclosure can have an accuracy of greater than about 50%, or greater than about 60%, or greater than about 70%, or greater than about 75%, or greater than about 80%, or greater than about 85%, or greater than about 90%, or greater than about 95%, or greater than about 99%.

In some aspects of the methods of the present disclosure, a predetermined cutoff value, for example a first predetermined cutoff value, is derived from a plurality of reference samples obtained from subjects not having or not diagnosed with a neoplastic disease. The plurality of reference samples can be about 2-500, 2-200, 10-100, or 20-80. Each reference sample produces a score using the algorithm, and the first predetermined cutoff value can be, for example, an arithmetic mean of these scores. Each reference sample can be blood, serum, plasma, or non-neoplastic tissue. In some aspects, each reference sample is blood. In some aspects, each reference sample is of the same type as the test sample.

In some aspects of the methods of the present disclosure, a test sample can comprise any biological fluid obtained from a subject. In some aspects, a test sample comprises blood, serum, plasma, neoplastic tissue or any combination thereof. In some aspects, a test sample comprises blood. In some aspects, a test sample comprises serum. In some aspects, a test sample comprises plasma.

In some aspects of the methods of the present disclosure, a reference sample can comprise any biological fluid obtained from a subject. In some aspects, a reference sample comprises blood, serum, plasma, neoplastic tissue or any combination thereof. In some aspects, a reference sample comprises blood. In some aspects, a reference sample comprises serum. In some aspects, a reference sample comprises plasma.

Each of the biomarkers disclosed herein may have one or more transcript variants. The methods disclosed herein can measure the expression level of any one of the transcript variants for each biomarker.

The expression level can be measured in a number of ways, including, but not limited to: measuring the mRNA encoded by the selected genes; measuring the amount of protein encoded by the selected genes; and measuring the activity of the protein encoded by the selected genes.

The biomarker can be RNA, cDNA, or protein. When the biomarker is RNA, the RNA can be reverse transcribed to produce cDNA (such as by RT-PCR), and the produced cDNA expression level is detected. The expression level of the biomarker can be detected by forming a complex between the biomarker and a labeled probe or primer. When the biomarker is RNA or cDNA, the RNA or cDNA is detected by forming a complex between the RNA or cDNA and a labeled nucleic acid probe or primer. The complex between the RNA or cDNA and the labeled nucleic acid probe or primer can be a hybridization complex.

Gene expression can also be detected by microarray analysis. Differential gene expression can also be identified, or confirmed using the microarray technique. Thus, the expression profile biomarkers can be measured in either fresh or fixed tissue, using microarray technology. In this method, polynucleotide sequences of interest (including cDNAs and oligonucleotides) are plated, or arrayed, on a microchip substrate. The arrayed sequences are then hybridized with specific DNA probes from cells or tissues of interest. The source of mRNA typically is total RNA isolated from a biological sample, and corresponding normal tissues or cell lines may be used to determine differential expression.

In some embodiments of the microarray technique, PCR amplified inserts of cDNA clones are applied to a substrate in a dense array. In some embodiments, at least 10,000 nucleotide sequences are applied to the substrate. The microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the microarray chip is scanned by a device such as, confocal laser microscopy or by another detection method, such as a CCD camera. Quantitation of hybridization of each arrayed element allows for assessment of corresponding mRNA abundance.

With dual color fluorescence, separately labeled cDNA probes generated from two sources of RNA are hybridized pair-wise to the array. The relative abundance of the transcripts from the two sources corresponding to each specified gene is thus determined simultaneously. Microarray analysis can be performed by commercially available equipment, following manufacturer's protocols.

In some embodiments, the biomarkers can be detected in a biological sample using qRT-PCR. The first step in gene expression profiling by RT-PCR is extracting RNA from a biological sample followed by the reverse transcription of the RNA template into cDNA and amplification by a PCR reaction. The reverse transcription reaction step is generally primed using specific primers, random hexamers, or oligo-dT primers, depending on the goal of expression profiling. The two commonly used reverse transcriptases are avilo myeloblastosis virus reverse transcriptase (AMV-RT) and Moloney murine leukemia virus reverse transcriptase (MLV-RT).

When the biomarker is protein, the protein can be detected by forming a complex between the protein and a labeled antibody. The label can be any label for example a fluorescent label, chemiluminescence label, radioactive label, etc. Exemplary methods for protein detection include, but are not limited to, enzyme immunoassay (EIA), radioimmunoassay (RIA), Western blot analysis and enzyme linked immunoabsorbant assay (ELISA). For example, the biomarker can be detected in an ELISA, in which the biomarker antibody is bound to a solid phase and an enzyme-antibody conjugate is employed to detect and/or quantify biomarker present in a sample. Alternatively, a western blot assay can be used in which solubilized and separated biomarker is bound to nitrocellulose paper. The combination of a highly specific, stable liquid conjugate with a sensitive chromogenic substrate allows rapid and accurate identification of samples.

In some aspects of the methods of the present disclosure, the methods described herein can have a specificity, sensitivity, and/or accuracy of at least 75%, 80%, 85%, 90%, 91%, 92%, 93%, 94%, 95%, 96%, 97%, 98%, or 99%.

In some aspects of the methods of the present disclosure, a labeled probe, a labeled primer, a labeled antibody or a labeled nucleic acid can comprise a fluorescent label.

Any algorithm that can generate a score for a sample by assessing where that sample value falls onto a prediction model generated using different techniques, e.g., decision trees, can be used in the methods disclosed herein. The algorithm analyzes the data (i.e., expression levels) and then assigns a score. In some embodiments, the algorithm can be a machine-learning algorithm. Exemplary algorithms that can be used in the methods disclosed herein can include, but are not limited to, XGBoost (XGB), Random Forest (RF), glmnet, cforest, Classification and Regression Trees for Machine Learning (CART), treebag, K-Nearest Neighbors (kNN), neural network (nnet), Support Vector Machine radial (SVM-radial), Support Vector Machine linear (SVM-linear), Naïve Bayes (NB), multilayer perceptron (mlp) or any combination thereof.

In some aspects of the methods of the present disclosure, the algorithm can be XGB (also called XGBoost). XGB is an implementation of gradient boosted decision trees designed for speed and performance.

In some aspects of the methods of the present disclosure, a therapy, for example a first therapy or a second therapy, can comprise active surveillance, surgery, radiation therapy, cryotherapy, hormone therapy, chemotherapy, vaccine treatment, bone-directed treatment, immunotherapy or any combination thereof.

In some aspects of the methods of the present disclosure, active surveillance can comprise a doctor visit with a prostate-specific antigen blood test and digital rectal exam about every 6 months. Active surveillance can also comprise prostate biopsies, which may be done every year.

In some aspects of the methods of the present disclosure, surgery can comprise a radical prostatectomy.

In some aspects of the methods of the present disclosure, radiation therapy can comprise external beam radiation and brachytherapy.

Cryotherapy, also referred to as cryosurgery or cryoablation, is the use of very cold temperatures to freeze and kill prostate cancer cells.

In some aspects of the methods of the present disclosure, hormone therapy can comprise androgen deprivation therapy or androgen suppression therapy. The goal is to reduce levels of male hormones, called androgens, in the body, or to stop them from affecting prostate cancer cells. Hormone therapy can comprise orchiectomy. Hormone therapy can also comprise administration of a compound that lowers the level of androgens, such as Luteinizing hormone-releasing hormone (LHRH) agonists, LHRH antagonists, and CYP17 inhibitors. Known LHRH agonists include, but are not limited to, leuprolide, goserelin, triptorelin, and histrelin. Known LHRH antagonists include degarelix. Known CYP17 inhibitors include abiraterone. Hormone therapy can also comprise administration of an anti-androgen, such as flutamide, bicalutamide, nilutamide, and enzalutamide. Hormone therapy can also include administration of an androgen-suppressing drug, such as estrogens and ketoconazole.

In some aspects of the methods of the present disclosure, chemotherapy can comprise docetaxel, cabazitaxel, mitoxantrone, estramustine, or a combination thereof.

In some aspects of the methods of the present disclosure, vaccine treatment can comprise Sipuleucel-T.

If the cancer has grown outside the prostate, preventing or slowing the spread of the cancer to the bones is a major goal of treatment. Bone-directed treatment can comprise bisphosphonates (e.g., zoledronic acid), denosumab, corticosteroids, external radiation therapy, radiopharmaceuticals (e.g., Strontium-89, Samarium-153, or Radium-223), and pain medicines.

The response of a subject having a prostate cancer to a therapy can also be evaluated by comparing the scores determined by the same algorithm at different time points of the therapy. For example, the first time point can be prior to or after the administration of the therapy to the subject; the second time point is after the first time point and after the administration of the therapy to the subject. A first score is generated at the first time point, and a second score is generated at the second time point. When the second score is significantly decreased as compared to the first score, the subject is considered to be responsive to the therapy. In some embodiments, the second score is significantly decreased as compared to the first score when the second score is at least 10% less than the first score, e.g., at least 20% less than the first score, at least 25% less than the first score, at least 40% less than the first score, at least 50% less than the first score, at least 75% less than the first score, or at least 90% less than the first score. When the second score is not significantly decreased as compared to the first score, the subject is considered to be not responsive to the therapy.

The sequence information of the prostate cancer biomarkers and housekeeper genes is shown in Table 1.

TABLE 1
Prostate Cancer Biomarker/Housekeeper Sequence Information

| | | | SEQ |
|---|---|---|---|
| Gene | RefSeq | | ID |
| Name | Accession | Sequence | NO: |
| AAMP | NM_001087.4 | ctgctggggagtcgcctacgcctacttcctgccgggaggaggggctcgagttccgcgtcgtc | 1 |
| | | gcgcagagctgactctgggaggcgtttgggcccagagaagtggatccgccgcttgcgccgc | |
| | | atggagtccgaatcggaaagcggggctgctgctgacacccccccactggagaccctaagct | |
| | | tccatggtgatgaagagattatcgaggtggtagaacttgatcccggtccgccggacccagatg | |
| | | acctggcccaggagatggaagatgtggactttgaggaagaagaggaggaagagggcaac | |
| | | gaagagggctgggttctagaaccccaggaaggggtggtcggcagcatggagggccccga | |
| | | cgatagcgaggtcacctttgcattgcactcagcatctgtgttttgtgtgagcctggaccccaag | |
| | | accaataccttggcagtgaccgggggtgaagatgacaaagccttcgtatggcggctcagcg | |
| | | atggggagctgctctttgagtgtgcaggccataaagactctgtgacttgtgctggtttcagccat | |
| | | gactccactctagtggccacaggggacatgagtggcctcttgaaagtgtggcaggtggacac | |
| | | taaggaggaggtctggtcctttgaagcgggagacctggagtggatggagtggcatcctcgg | |
| | | gcacctgtcctgttggcgggcacagctgacggcaacacctggatgtggaaagtcccgaatg | |
| | | gtgactgcaagaccttccagggtcccaactgcccagccacctgtggccgagtcctccctgat | |
| | | gggaagagagctgtggtaggctatgaagatgggaccatcaggatttgggacctgaagcagg | |
| | | gaagccctatccatgtactgaaagggactgagggtcaccagggcccactcacctgtgttgct | |
| | | gccaaccaggatggcagcttgatcctaactggctctgtggactgccaggccaagctggtcag | |
| | | tgccaccaccggcaaggtggtgggtgtttttagacctgagactgtggcctcccagcccagcct | |
| | | gggagaaggggaggagagtgagtccaactcggtggagtccttgggcttctgcagtgtgatg | |
| | | cccctggcagctgttggctacctggatgggaccttggccatctatgacctggctacgcagact | |
| | | cttaggcatcagtgtcagcaccagtcgggcatcgtgcagctgctgtgggaggcaggcactgc | |
| | | cgtggtatatacctgcagcctggatggcatcgtgcgcctctgggacgcccggaccggccgc | |
| | | ctgcttactgactaccggggccacacggctgagatcctggactttgccctcagcaaagatgcc | |
| | | tccctggtggtgaccacgtcaggagaccacaaagcgaaagtattttgtgtccaaaggcctgac | |
| | | cgttaatggctgcagcccctgcctgtgtgtctggtgttgaggggacgaagggacccctgccc | |
| | | ctgtctgccagcagaggcagtagggcacagagggaagaggagggtggggccctggatga | |
| | | ctttccagcctcttcaactgacttgctcccctctccttttcttctctttagagacccagcccagggc | |
| | | cctcccacccttgtccagacctggtgggcccttcagagggaggggtggacctgtttctctttca | |
| | | ctttcatttgctggtgtgagccatggggtgtgtatttgtatgtggggagtaggtgtttgaggttcc | |
| | | cgttctttcccttcccaagtctctgggggtggaaaggaggaagagatactagttaaagattttaa | |
| | | aaatgtaaataaaatatacttcccagaaaaaaaaaaa | |
| ANO7 | NM_001001891.3 | aaagatagatcctgctccaggagccgggaagccttgccctggccagctgtgctgggcacctc | 2 |
| | | ccctgcctgcttcctggcccacttgcaggcaaggtgagggcatgcgaatggctgccactgcc | |
| | | tgggcggggctccaagggccacccctccccaccctctgtcccgcagtgaggacgggactct | |
| | | actgccgagaccaggctcacgctgagaggtgggccatgacctccgagacctcttccggaag | |
| | | ccactgtgccaggagcaggatgctgcggcgacgggcccaggaagaggacagcaccgtcc | |
| | | tgatcgatgtgagcccccctgaggcagagaagaggggctcttacgggagcacagcccacg | |
| | | cctcggagccaggtggacagcaagcggccgcctgcagagctgggagtcctgccaagccc | |
| | | cggatcgcagacttcgtcctcgtttgggaggaggacctgaagctagacaggcagcaggaca | |
| | | gtgccgcccgggacagaacagacatgcacaggacctggcgggagacttttctggataatctt | |
| | | cgtgcggctgggctgtgtgtagaccagcaggacgtccaggacgggaacaccacagtgcac | |
| | | tacgccctcctcagcgcctcctgggctgtgctctgctactacgccgaagacctgcgcctgaag | |
| | | ctgcccttgcaggagttacccaaccaggcctccaactggtcggccggcctgctggcatggct | |
| | | gggcatccccaacgtcctgctggaggttgtgccagacgtaccccccgagtactactcctgcc | |
| | | ggttcagagtgaacaagctgccacgcttcctcgggagtgacaaccaggacaccttcttcacaa | |
| | | gcaccaagaggcaccaaattctgtttgagatcctggccaagaccccgtatggccacgagaag | |
| | | aaaaacctgcttgggatccaccagctgctggcagagggtgtcctcagtgccgccttccccctg | |
| | | catgacggccccttcaagacgcccccagagggcccgcaggctccacgcctcaaccagcgc | |
| | | caagtccttttccagcactgggcgcgctggggcaagtggaacaagtaccagcccctggacc | |
| | | acgtgcgcaggtacttcggggagaaggtggccctctacttcgcctggctcgggttttacacag | |
| | | gctggctcctgccagcggcagtggtgggcacactggtgttcctggtgggctgcttcctggtgt | |
| | | tctcagacatacccacgcaggaactgtgtggcagcaaggacagcttcgagatgtgcccacttt | |
| | | gcctcgactgccctttctggctgctctccagcgcctgtgccctggcccaggccggccggctgt | |
| | | tcgaccacggcggcaccgtgttcttcagcttgttcatggcactgtgggccgtgctgctgctgga | |
| | | gtactggaagcggaagagcgccacgctggcctaccgctgggactgctctgactacgaggac | |
| | | actgaggagaggcctcggccccagtttgccgcctcagcccccatgacagccccgaacccca | |
| | | tcacgggtgaggacgagccctacttccctgagaggagccgcgcgcgccgcatgctggccg | |
| | | gctctgtggtgatcgtggtgatggtggccgtggtggtcatgtgcctcgtgtctatcatcctgtac | |
| | | cgtgccatcatggccatcgtggtgtccaggtcgggcaacacccttctcgcagcctgggcctct | |
| | | cgcatcgccagcctcacggggtctgtagtgaacctcgtcttcatcctcatcctctccaagatcta | |
| | | tgtatccctggcccacgtcctgacacgatgggaaatgcaccgcacccagaccaagttcgagg | |
| | | acgccttcaccctcaaggtgttcatcttccagttcgtcaacttctactcctcacccgtctacattgc | |
| | | cttcttcaagggcaggtttgtgggatacccaggcaactaccacaccttgtttggagtccgcaat | |
| | | gaggagtgcgcggctggaggctgcctgatcgagctggcacaggagctcctggtcatcatgg | |
| | | tgggcaagcaggtcatcaacaacatgcaggaggtcctcatcccgaagctaaagggctggtg | |
| | | gcagaagttccggcttcgctccaagaagaggaaggcgggagcttctgcaggggctagcca | |
| | | ggggccctgggaggacgactatgagcttgtgccctgtgagggtctgtttgacgagtacctgg | |
| | | aaatggtgctgcagttcggcttcgtcaccatcttcgtggccgcctgtccgctcgcgccgctctt | |
| | | cgccctgctcaacaactgggtggagatccgcttggacgcgcgcaagttcgtctgcgagtacc | |
| | | ggcgcccggtggccgagcgcgcccaggacatcggcatctggttccacatcctggcgggcc | |
| | | tcacgcacctggcggtcatcagcaacgccttcctcctggccttctcgtccgacttcctgccgcg | |
| | | cgcctactaccggtggacccgcgcccacgacctgcgcggcttcctcaacttcacgctggcgc | |
| | | gagccccgtcctccttcgccgccgcgcacaaccgcacgtgcaggtatcgggctttccgggat | |
| | | gacgatggacattattcccagacctactggaatcttcttgccatccgcctggccttcgtcattgtg | |
| | | tttgagcatgtggttttctccgttggccgcctcctggacctcctggtgcctgacatcccagagtct | |
| | | gtggagatcaaagtgaagcgggagtactacctggctaagcaggcactggctgagaatgagg | |
| | | ttctttttggaacgaacggaacaaaggatgagcagcccgagggctcagagctcagctcccac | |
| | | tggacacccttcacggttcccaaggccagccagctgcagcagtgacgcctggaaggacatct | |
| | | ggtggtccttaggggagtggcccctcctgagccctgcgagcagcgtccttttcctcttccctca | |
| | | ggcagcggctgtgtgaaccgctggctgctgttgtgcctcatctctgggcacattgcctgcttcc | |
| | | ccccagcgccggcttctctcctcagagcgcctgtcactccatccccggcagggagggaccgt | |
| | | cagctcacaaggccctctttgtttcctgctcccagacataagcccaaggggcccctgcaccca | |
| | | agggaccctgtccctcggtggcctccccaggcccctggacacgacagttctcctcaggcag | |
| | | gtgggctttgtggtcctcgccgcccctggccacatcgccctctcctcttacacctggtgaccttc | |
| | | gaatgtttcagagcgcagggccgttctccctcgtgtcctctggacccacccgccccttcctgcc | |
| | | ctgtttgcgcagggacatcacccacatgccccagctctcggaccctgcagctctgtgtcccag | |
| | | gccacagcaaaggtctgttgaacccctccctccattcccagttatctgggtcctctggattcttct | |
| | | gtttcttgaatcaggctctgctttccccctagccactacaggcagcctctgacagtgccgcttta | |
| | | cttgcattctgcagcaattacatgtgtccttttgatccttgcccaacttccctccctctcccagctcc | |
| | | tggcccctggcccagggcccctcttgctgtttttacctctgttccttggggcctagtacccagca | |
| | | agcacccaaatgggggaggttttgggatgagaggaggaaacgtgtatacctgtaacatctgg | |
| | | tggctcttcccccagaagtttgtgttcatacataattgttttccacgctggatcataatgtgacgtg | |
| | | cagttctgccctgtgctggggagccacatgaagcttcccctggctaacttgctaccccgcagc | |
| | | aatcccagtgtggccgtctgcttgctaaaaaatggatctgtgctcatctgtattgatgtccttgga | |
| | | gttctacaagtggaacttaagtgtcaaaaagaatatgtggtttttagctgagcgtggtggctcac | |
| | | acctgtaatcccaggactttgggaggctgaggcaggaggattacaaggtcaggagttcggg | |
| | | actagcctgtccaacatggtgaaaccctgtctttactaaaaatgcaaaaatt | |
| AR | NM_000044.3 | cgagatcccggggagccagcttgctgggagagcgggacggtccggagcaagcccagag | 3 |
| | | gcagaggaggcgacagagggaaaaagggccgagctagccgctccagtgctgtacaggag | |
| | | ccgaagggacgcaccacgccagccccagcccggctccagcgacagccaacgcctcttgca | |
| | | gcgcggcggcttcgaagccgccgcccggagctgccctttcctcttcggtgaagtttttaaaag | |
| | | ctgctaaagactcggaggaagcaaggaaagtgcctggtaggactgacggctgcctttgtcct | |
| | | cctcctctccaccccgcctccccccaccctgccttccccccctcccccgtcttctctcccgcag | |
| | | ctgcctcagtcggctactctcagccaacccccctcaccacccttctccccacccgcccccccg | |
| | | cccccgtcggcccagcgctgccagcccgagtttgcagagaggtaactccctttggctgcgag | |
| | | cgggcgagctagctgcacattgcaaagaaggctcttaggagccaggcgactggggagcgg | |
| | | cttcagcactgcagccacgacccgcctggttaggctgcacgcggagagaaccctctgttttcc | |
| | | cccactctctctccacctcctcctgccttccccaccccgagtgcggagccagagatcaaaaga | |
| | | tgaaaaggcagtcaggtcttcagtagccaaaaaacaaaacaaacaaaaacaaaaaagccga | |
| | | aataaaagaaaaagataataactcagttcttatttgcacctacttcagtggacactgaatttggaa | |
| | | ggtggaggattttgtttttttcttttaagatctgggcatcttttgaatctacccttcaagtattaagag | |
| | | acagactgtgagcctagcagggcagatcttgtccaccgtgtgtcttcttctgcacgagactttg | |
| | | aggctgtcagagcgctttttgcgtggttgctcccgcaagtttccttctctggagcttcccgcagg | |
| | | tgggcagctagctgcagcgactaccgcatcatcacagcctgttgaactcttctgagcaagaga | |
| | | aggggaggcggggtaagggaagtaggtggaagattcagccaagctcaaggatggaagtg | |
| | | cagttagggctgggaagggtctaccctcggccgccgtccaagacctaccgaggagctttcca | |
| | | gaatctgttccagagcgtgcgcgaagtgatccagaacccgggccccaggcacccagaggc | |
| | | cgcgagcgcagcacctcccggcgccagtttgctgctgctgcagcagcagcagcagcagca | |
| | | gcagcagcagcagcagcagcagcagcagcagcagcagcagcagcagcaagagactagc | |
| | | cccaggcagcagcagcagcagcagggtgaggatggttctccccaagcccatcgtagaggc | |
| | | cccacaggctacctggtcctggatgaggaacagcaaccttcacagccgcagtcggccctgg | |
| | | agtgccaccccgagagaggttgcgtcccagagcctggagccgccgtggccgccagcaag | |
| | | gggctgccgcagcagctgccagcacctccggacgaggatgactcagctgccccatccacgt | |
| | | tgtccctgctgggccccactttccccggcttaagcagctgctccgctgaccttaaagacatcct | |
| | | gagcgaggccagcaccatgcaactccttcagcaacagcagcaggaagcagtatccgaagg | |
| | | cagcagcagcgggagagcgagggaggcctcgggggctcccacttcctccaaggacaatta | |
| | | cttagggggcacttcgaccatttctgacaacgccaaggagttgtgtaaggcagtgtcggtgtc | |
| | | catgggcctgggtgtggaggcgttggagcatctgagtccaggggaacagcttcggggggat | |
| | | tgcatgtacgccccacttttgggagttccacccgctgtgcgtcccactccttgtgccccattggc | |
| | | cgaatgcaaaggttctctgctagacgacagcgcaggcaagagcactgaagatactgctgagt | |
| | | attcccctttcaagggaggttacaccaaagggctagaaggcgagagcctaggctgctctggc | |
| | | agcgctgcagcagggagctccgggacacttgaactgccgtctaccctgtctctctacaagtcc | |
| | | ggagcactggacgaggcagctgcgtaccagagtcgcgactactacaactttccactggctct | |
| | | ggccggaccgccgccccctccgccgcctccccatccccacgctcgcatcaagctggagaa | |
| | | cccgctggactacggcagcgcctgggcggctgcggcggcgcagtgccgctatggggacct | |
| | | ggcgagcctgcatggcgcgggtgcagcgggacccggttctgggtcaccctcagccgccgc | |
| | | ttcctcatcctggcacactctcttcacagccgaagaaggccagttgtatggaccgtgtggtggt | |
| | | ggtgggggtggtggcggcggcggcggcggcggcggcggcggcggcggcggcggcgg | |
| | | cggcggcgaggcgggagctgtagccccctacggctacactcggccccctcaggggctggc | |
| | | gggccaggaaagcgacttcaccgcacctgatgtgtggtaccctggcggcatggtgagcaga | |
| | | gtgccctatcccagtcccacttgtgtcaaaagcgaaatgggcccctggatggatagctactcc | |
| | | ggaccttacggggacatgcgtttggagactgccagggaccatgttttgcccattgactattactt | |
| | | tccaccccagaagacctgcctgatctgtggagatgaagcttctgggtgtcactatggagctctc | |
| | | acatgtggaagctgcaaggtcttcttcaaaagagccgctgaagggaaacagaagtacctgtg | |
| | | cgccagcagaaatgattgcactattgataaattccgaaggaaaaattgtccatcttgtcgtcttc | |
| | | ggaaatgttatgaagcagggatgactctgggagcccggaagctgaagaaacttggtaatctg | |
| | | aaactacaggaggaaggagaggcttccagcaccaccagccccactgaggagacaaccca | |
| | | gaagctgacagtgtcacacattgaaggctatgaatgtcagcccatctttctgaatgtcctggaa | |
| | | gccattgagccaggtgtagtgtgtgctggacacgacaacaaccagcccgactcctttgcagc | |
| | | cttgctctctagcctcaatgaactgggagagagacagcttgtacacgtggtcaagtgggccaa | |
| | | ggccttgcctggcttccgcaacttacacgtggacgaccagatggctgtcattcagtactcctgg | |
| | | atggggctcatggtgtttgccatgggctggcgatccttcaccaatgtcaactccaggatgctct | |
| | | acttcgcccctgatctggttttcaatgagtaccgcatgcacaagtcccggatgtacagccagtg | |
| | | tgtccgaatgaggcacctctctcaagagtttggatggctccaaatcaccccccaggaattcctg | |
| | | tgcatgaaagcactgctactcttcagcattattccagtggatgggctgaaaaatcaaaaattcttt | |
| | | gatgaacttcgaatgaactacatcaaggaactcgatcgtatcattgcatgcaaaagaaaaaatc | |
| | | ccacatcctgctcaagacgcttctaccagctcaccaagctcctggactccgtgcagcctattgc | |
| | | gagagagctgcatcagttcacttttgacctgctaatcaagtcacacatggtgagcgtggactttc | |
| | | cggaaatgatggcagagatcatctctgtgcaagtgcccaagatcctttctgggaaagtcaagc | |
| | | ccatctatttccacacccagtgaagcattggaaaccctatttccccaccccagctcatgccccct | |
| | | ttcagatgtcttctgcctgttataactctgcactactcctctgcagtgccttggggaatttcctctat | |
| | | tgatgtacagtctgtcatgaacatgttcctgaattctatttgctgggctttttttttctctttctctccttt | |
| | | ctttttcttcttccctccctatctaaccctcccatggcaccttcagactttgcttcccattgtggctcc | |
| | | tatctgtgttttgaatggtgttgtatgcctttaaatctgtgatgatcctcatatggcccagtgtcaag | |
| | | ttgtgcttgtttacagcactactctgtgccagccacacaaacgtttacttatcttatgccacggga | |
| | | agtttagagagctaagattatctggggaaatcaaaacaaaaacaagcaaacaaaaaaaaaaa | |
| | | gcaaaaacaaaacaaaaaataagccaaaaaaccttgctagtgttttttcctcaaaaataaataaa | |
| | | taaataaataaatacgtacatacatacacacatacatacaaacatatagaaatccccaaagagg | |
| | | ccaatagtgacgagaaggtgaaaattgcaggcccatggggagttactgattttttcatctcctcc | |
| | | ctccacgggagactttattttctgccaatggctattgccattagagggcagagtgaccccagag | |
| | | ctgagttgggcaggggggtggacagagaggagaggacaaggagggcaatggagcatca | |
| | | gtacctgcccacagccttggtccctgggggctagactgctcaactgtggagcaattcattatac | |
| | | tgaaaatgtgcttgttgttgaaaatttgtctgcatgttaatgcctcacccccaaacccttttctctct | |
| | | cactctctgcctccaacttcagattgactttcaatagtttttctaagacctttgaactgaatgttctct | |
| | | tcagccaaaacttggcgacttccacagaaaagtctgaccactgagaagaaggagagcagag | |
| | | atttaaccctttgtaaggccccatttggatccaggtctgctttctcatgtgtgagtcagggagga | |
| | | gctggagccagaggagaagaaaatgatagcttggctgttctcctgcttaggacactgactgaa | |
| | | tagttaaactctcactgccactaccttttccccacctttaaaagacctgaatgaagttttctgccaa | |
| | | actccgtgaagccacaagcaccttatgtcctcccttcagtgttttgtgggcctgaatttcatcaca | |
| | | ctgcatttcagccatggtcatcaagcctgtttgcttcttttgggcatgttcacagattctctgttaag | |
| | | agcccccaccaccaagaaggttagcaggccaacagctctgacatctatctgtagatgccagt | |
| | | agtcacaaagatttcttaccaactctcagatcgctggagcccttagacaaactggaaagaagg | |
| | | catcaaagggatcaggcaagctgggcgtcttgcccttgtcccccagagatgataccctccca | |
| | | gcaagtggagaagttctcacttccttctttagagcagctaaaggggctacccagatcagggttg | |
| | | aagagaaaactcaattaccagggtgggaagaatgaaggcactagaaccagaaaccctgcaa | |
| | | atgctcttcttgtcacccagcatatccacctgcagaagtcatgagaagagagaaggaacaaag | |
| | | aggagactctgactactgaattaaaatcttcagcggcaaagcctaaagccagatggacaccat | |
| | | ctggtgagtttactcatcatcctcctctgctgctgattctgggctctgacattgcccatactcactc | |
| | | agattccccacctttgttgctgcctcttagtcagagggaggccaaaccattgagactttctacag | |
| | | aaccatggcttctttcggaaaggtctggttggtgtggctccaatactttgccacccatgaactca | |
| | | gggtgtgccctgggacactggttttatatagtcttttggcacacctgtgttctgttgacttcgttctt | |
| | | caagcccaagtgcaagggaaaatgtccacctactttctcatcttggcctctgcctccttacttag | |
| | | ctcttaatctcatctgttgaactcaagaaatcaagggccagtcatcaagctgcccattttaattgat | |
| | | tcactctgtttgttgagaggatagtttctgagtgacatgatatgatccacaagggtttccttccctg | |
| | | atttctgcattgatattaatagccaaacgaacttcaaaacagctttaaataacaagggagaggg | |
| | | gaacctaagatgagtaatatgccaatccaagactgctggagaaaactaaagctgacaggttcc | |
| | | ctttttggggtgggatagacatgttctggttttctttattattacacaatctggctcatgtacaggat | |
| | | cacttttagctgttttaaacagaaaaaaatatccaccactcttttcagttacactaggttacattttaa | |
| | | taggtcctttacatctgttttggaatgattttcatcttttgtgatacacagattgaattatatcattttca | |
| | | tatctctccttgtaaatactagaagctctcctttacatttctctatcaaatttttcatctttatgggtttc | |
| | | ccaattgtgactcttgtcttcatgaatatatgtttttcatttgcaaaagccaaaaatcagtgaaaca | |
| | | gcagtgtaattaaaagcaacaactggattactccaaatttccaaatgacaaaactagggaaaaa | |
| | | tagcctacacaagcctttaggcctactctttctgtgcttgggtttgagtgaacaaaggagatttta | |
| | | gcttggctctgttctcccatggatgaaaggaggaggattttttttttcttttggccattgatgttcta | |
| | | gccaatgtaattgacagaagtctcattttgcatgcgctctgctctacaaacagagttggtatggtt | |
| | | ggtatactgtactcacctgtgagggactggccactcagacccacttagctggtgagctagaag | |
| | | atgaggatcactcactggaaaagtcacaaggaccatctccaaacaagttggcagtgctcgatg | |
| | | tggacgaagagtgaggaagagaaaaagaaggagcaccagggagaaggctccgtctgtgct | |
| | | gggcagcagacagctgccaggatcacgaactctgtagtcaaagaaaagagtcgtgtggcag | |
| | | tttcagctctcgttcattgggcagctcgcctaggcccagcctctgagctgacatgggagttgttg | |
| | | gattctttgtttcatagctttttctatgccataggcaatattgttgttcttggaaagtttattatttttttaa | |
| | | ctcccttactctgagaaagggatattttgaaggactgtcatatatctttgaaaaaagaaaatctgt | |
| | | aatacatatatttttatgtatgttcactggcactaaaaaatatagagagcttcattctgtcctttgggt | |
| | | agttgctgaggtaattgtccaggttgaaaaataatgtgctgatgctagagtccctctctgtccata | |
| | | ctctacttctaaatacatataggcatacatagcaagttttatttgacttgtactttaagagaaaatat | |
| | | gtccaccatccacatgatgcacaaatgagctaacattgagcttcaagtagcttctaagtgtttgtt | |
| | | tcattaggcacagcacagatgtggcctttccccccttctctcccttgatatctggcagggcataa | |
| | | aggcccaggccacttcctctgccccttcccagccctgcaccaaagctgcatttcaggagactc | |
| | | tctccagacagcccagtaactacccgagcatggcccctgcatagccctggaaaaataagagg | |
| | | ctgactgtctacgaattatcttgtgccagttgcccaggtgagagggcactgggccaagggagt | |
| | | ggttttcatgtttgacccactacaaggggtcatgggaatcaggaatgccaaagcaccagatca | |
| | | aatccaaaacttaaagtcaaaataagccattcagcatgttcagtttcttggaaaaggaagtttcta | |
| | | cccctgatgcctttgtaggcagatctgttctcaccattaatctttttgaaaatcttttaaagcagtttt | |
| | | taaaaagagagatgaaagcatcacattatataaccaaagattacattgtacctgctaagatacc | |
| | | aaaattcataagggcagggggggagcaagcattagtgcctctttgataagctgtccaaagac | |
| | | agactaaaggactctgctggtgactgacttataagagctttgtgggtttttttttccctaataatata | |
| | | catgtttagaagaattgaaaataatttcgggaaaatgggattatgggtccttcactaagtgatttta | |
| | | taagcagaactggctttccttttctctagtagttgctgagcaaattgttgaagctccatcattgcat | |
| | | ggttggaaatggagctgttcttagccactgtgtttgctagtgcccatgttagcttatctgaagatg | |
| | | tgaaacccttgctgataagggagcatttaaagtactagattttgcactagagggacagcaggc | |
| | | agaaatccttatttctgcccactttggatggcacaaaaagttatctgcagttgaaggcagaaagt | |
| | | tgaaatacattgtaaatgaatatttgtatccatgtttcaaaattgaaatatatatatatatatatatata | |
| | | tatatatatatatatatagtgtgtgtgtgtgttctgatagctttaactttctctgcatctttatatttggtt | |
| | | ccagatcacacctgatgccatgtacttgtgagagaggatgcagttttgttttggaagctctctca | |
| | | gaacaaacaagacacctggattgatcagttaactaaaagttttctcccctattgggtttgaccca | |
| | | caggtcctgtgaaggagcagagggataaaaagagtagaggacatgatacattgtactttacta | |
| | | gttcaagacagatgaatgtggaaagcataaaaactcaatggaactgactgagatttaccacag | |
| | | ggaaggcccaaacttggggccaaaagcctacccaagtgattgaccagtggccccctaatgg | |
| | | gacctgagctgttggaagaagagaactgttccttggtcttcaccatccttgtgagagaagggc | |
| | | agtttcctgcattggaacctggagcaagcgctctatctttcacacaaattccctcacctgagattg | |
| | | aggtgctcttgttactgggtgtctgtgtgctgtaattctggttttggatatgttctgtaaagattttga | |
| | | caaatgaaaatgtgtttttctctgttaaaacttgtcagagtactagaagttgtatctctgtaggtgc | |
| | | aggtccatttctgcccacaggtagggtgtttttctttgattaagagattgacacttctgttgcctag | |
| | | gacctcccaactcaaccatttctaggtgaaggcagaaaaatccacattagttactcctcttcaga | |
| | | catttcagctgagataacaaatcttttggaattttttcacccatagaaagagtggtagatatttgaa | |
| | | tttagcaggtggagtttcatagtaaaaacagcttttgactcagctttgatttatcctcatttgatttgg | |
| | | ccagaaagtaggtaatatgcattgattggcttctgattccaattcagtatagcaaggtgctaggtt | |
| | | ttttcctttccccacctgtctcttagcctggggaattaaatgagaagccttagaatgggtggccct | |
| | | tgtgacctgaaacacttcccacataagctacttaacaagattgtcatggagctgcagattccatt | |
| | | gcccaccaaagactagaacacacacatatccatacaccaaaggaaagacaattctgaaatgc | |
| | | tgtttctctggtggttccctctctggctgctgcctcacagtatgggaacctgtactctgcagaggt | |
| | | gacaggccagatttgcattatctcacaaccttagcccttggtgctaactgtcctacagtgaagtg | |
| | | cctggggggttgtcctatcccataagccacttggatgctgacagcagccaccatcagaatgac | |
| | | ccacgcaaaaaaaagaaaaaaaaaattaaaaagtcccctcacaacccagtgacacctttctgc | |
| | | tttcctctagactggaacattgattagggagtgcctcagacatgacattcttgtgctgtccttgga | |
| | | attaatctggcagcaggagggagcagactatgtaaacagagataaaaattaattttcaatattg | |
| | | aaggaaaaaagaaataagaagagagagagaaagaaagcatcacacaaagattttcttaaaa | |
| | | gaaacaattttgcttgaaatctctttagatggggctcatttctcacggtggcacttggcctccact | |
| | | gggcagcaggaccagctccaagcgctagtgttctgttctctttttgtaatcttggaatcttttgttg | |
| | | ctctaaatacaattaaaaatggcagaaacttgtttgttggactacatgtgtgactttgggtctgtct | |
| | | ctgcctctgctttcagaaatgtcatccattgtgtaaaatattggcttactggtctgccagctaaaac | |
| | | ttggccacatcccctgttatggctgcaggatcgagttattgttaacaaagagacccaagaaaag | |
| | | ctgctaatgtcctcttatcattgttgttaatttgttaaaacataaagaaatctaaaatttcaaaaaa | |
| AR-V7 | | gacactgaatttggaaggtggaggattttgtttttttcttttaagatctgggcatcttttgaatctacc | 4 |
| | | cttcaagtattaagagacagactgtgagcctagcagggcagatcttgtccaccgtgtgtcttctt | |
| | | ctgcacgagactttgaggctgtcagagcgctttttgcgtggttgctcccgcaagtttccttctctg | |
| | | gagcttcccgcaggtgggcagctagctgcagcgactaccgcatcatcacagcctgttgaact | |
| | | cttctgagcaagagaaggggaggcggggtaagggaagtaggtggaagattcagccaagct | |
| | | caaggatggaagtgcagttagggctgggaagggtctaccctcggccgccgtccaagaccta | |
| | | ccgaggagctttccagaatctgttccagagcgtgcgcgaagtgatccagaacccgggcccc | |
| | | aggcacccagaggccgcgagcgcagcacctcccggcgccagtttgctgctgcagcagcag | |
| | | cagcagcagcagcagcagcagcagcagcagcagcagcagcagcagcagcagcagcagc | |
| | | agcagcagcagcagcagcaagagactagccccaggcagcagcagcagcagcagggtga | |
| | | ggatggttctccccaagcccatcgtagaggccccacaggctacctggtcctggatgaggaac | |
| | | agcaaccttcacagccgcagtcggccctggagtgccaccccgagagaggttgcgtcccaga | |
| | | gcctggagccgccgtggccgccagcaaggggctgccgcagcagctgccagcacctccgg | |
| | | acgaggatgactcagctgccccatccacgttgtccctgctgggccccactttccccggcttaa | |
| | | gcagctgctccgctgaccttaaagacatcctgagcgaggccagcaccatgcaactccttcag | |
| | | caacagcagcaggaagcagtatccgaaggcagcagcagcgggagagcgagggaggcct | |
| | | cgggggctcccacttcctccaaggacaattacttagggggcacttcgaccatttctgacaacg | |
| | | ccaaggagttgtgtaaggcagtgtcggtgtccatgggcctgggtgtggaggcgttggagcat | |
| | | ctgagtccaggggaacagcttcggggggattgcatgtacgccccacttttgggagttccaccc | |
| | | gctgtgcgtcccactccttgtgccccattggccgaatgcaaaggttctctgctagacgacagc | |
| | | gcaggcaagagcactgaagatactgctgagtattcccctttcaagggaggttacaccaaagg | |
| | | gctagaaggcgagagcctaggctgctctggcagcgctgcagcagggagctccgggacact | |
| | | tgaactgccgtctaccctgtctctctacaagtccggagcactggacgaggcagctgcgtacca | |
| | | gagtcgcgactactacaactttccactggctctggccggaccgccgccccctccgccgcctc | |
| | | cccatccccacgctcgcatcaagctggagaacccgctggactacggcagcgcctgggcgg | |
| | | ctgcggcggcgcagtgccgctatggggacctggcgagcctgcatggcgcgggtgcagcg | |
| | | ggacccggttctgggtcaccctcagccgccgcttcctcatcctggcacactctcttcacagcc | |
| | | gaagaaggccagttgtatggaccgtgtggtggtggtgggggtggtggcggcggcggcggc | |
| | | ggcggcggcggcggcggcggcggcgaggcgggagctgtagccccctacggctacactc | |
| | | ggccccctcaggggctggcgggccaggaaagcgacttcaccgcacctgatgtgtggtaccc | |
| | | tggcggcatggtgagcagagtgccctatcccagtcccacttgtgtcaaaagcgaaatgggcc | |
| | | cctggatggatagctactccggaccttacggggacatgcgtttggagactgccagggaccat | |
| | | gttttgcccattgactattactttccaccccagaagacctgcctgatctgtggagatgaagcttct | |
| | | gggtgtcactatggagctctcacatgtggaagctgcaaggtcttcttcaaaagagccgctgaa | |
| | | gggaaacagaagtacctgtgcgccagcagaaatgattgcactattgataaattccgaaggaa | |
| | | aaattgtccatcttgtcgtcttcggaaatgttatgaagcagggatgactctgggagaaaaattcc | |
| | | gggttggcaattgcaagcatctcaaaatgaccagaccctgaagaaaggctgacttgcctcatt | |
| | | caaaatgagggctctagagggctctagtggatagtctggagaaacctggcgtctgaggctta | |
| | | ggagcttaggtttttgctcctcaacacagactttgacgttggggttgggggctactctcttgattg | |
| | | ctgactccctccagcgggaccaatagtgttttcctacctcacagggatgttgtgaggacgggct | |
| | | gtagaagtaatagtggttaccactcatgtagttgtgagtatcatgattattgtttcctgtaatgtgg | |
| | | cttggcattggcaaagtgctttttgattgttcttgatcacatatgatgggggccaggcactgactc | |
| | | aggcggatgcagtgaagctctggctcagtcgcttgcttttcgtggtgtgctgccaggaagaaa | |
| | | ctttgctgatgggactcaaggtgtcaccttggacaagaagcaactgtgtctgtctgaggttcctg | |
| | | tggccatctttatttgtgtattaggcaattcgtatttcccccttaggttctagccttctggatcccag | |
| | | ccagtgacctagatcttagcctcaggccctgtcactgagctgaaggtagtagctgatccacag | |
| | | aagttcagtaaacaaggaccagatttctgcttctccaggagaagaagccagccaacccctctc | |
| | | ttcaaacacactgagagactacagtccgactttccctcttacatctagccttactgtagccacact | |
| | | ccttgattgctctctcacatcacatgcttctcttcatcagttgtaagcctctcattcttctcccaagc | |
| | | cagactcaaatattgtattgatgtcaaagaagaatcacttagagtttggaatatcttgttctctctct | |
| | | gctccatagcttccatattgacaccagtttctttctagtggagaagtggagtctgtgaagccagg | |
| | | gaaacacacatgtgagagtcagaaggactctccctgacttgcctggggcctgtctttcccacct | |
| | | tctccagtctgtctaaacacacacacacacacacacacacacacacacacacacacacacgc | |
| | | tctctctctctctccccccccaacacacacacactctctctctcacacacacacacatacacaca | |
| | | cacttctttctctttcccctgactcagcaacattctggagaaaagccaaggaaggacttcagga | |
| | | ggggagtttcccccttctcagggcagaattttaatctccagaccaacaagaagttccctaatgt | |
| | | ggattgaaaggctaatgaggtttatttttaactactttctatttgtttgaatgttgcatatttctactagt | |
| | | gaaattttcccttaataaagccattaatacacccaaaaaaaaaaaaaaaa | |
| C16orf89 | NM_001098514.2 | ctacgcccaggactggcctaacctccgctgcctaaccgtcatccccctgcagccacccttccc | 5 |
| | | agagtcctttgcccaggccaccccaggcttcttggcagccctgccgggccacttgtcttcatgt | |
| | | ctgccagggggaggtgggaaggaggtgggaggagggcgtgcagaggcagtctgggcttg | |
| | | gccagagctcagggtgctgagcgtgtgaccagcagtgagcagaggccggccatggccag | |
| | | cctggggctgctgctcctgctcttactgacagcactgccaccgctgtggtcctcctcactgcct | |
| | | gggctggacactgctgaaagtaaagccaccattgcagacctgatcctgtctgcgctggagag | |
| | | agccaccgtcttcctagaacagaggctgcctgaaatcaacctggatggcatggtgggggtcc | |
| | | gagtgctggaagagcagctaaaaagtgtccgggagaagtgggcccaggagcccctgctgc | |
| | | agccgctgagcctgcgcgtggggatgctgggggagaagctggaggctgccatccagagat | |
| | | ccctccactacctcaagctgagtgatcccaagtacctaagagagttccagctgaccctccagc | |
| | | ccgggttttggaagctcccacatgcctggatccacactgatgcctccttggtgtaccccacgtt | |
| | | cgggccccaggactcattctcagaggagagaagtgacgtgtgcctggtgcagctgctggga | |
| | | accgggacggacagcagcgagccctgcggcctctcagacctctgcaggagcctcatgacc | |
| | | aagcccggctgctcaggctactgcctgtcccaccaactgctcttcttcctctgggccagaatga | |
| | | gggggtgcacacagggaccactccaacagagccaggactatatcaacctcttctgcgccaa | |
| | | catgatggacttgaaccgcagagctgaggccatcggatacgcctaccctacccgggacatct | |
| | | tcatggaaaacatcatgttctgtggaatgggcggcttctccgacttctacaagctccggtggct | |
| | | ggaggccattctcagctggcagaaacagcaggaaggatgcttcggggagcctgatgctgaa | |
| | | gatgaagaattatctaaagctattcaatatcagcagcatttttcgaggagagtgaagaggcgag | |
| | | aaaaacaatttccagatggctgctcctcccacaacacagccacagcagtggcagccctgggt | |
| | | ggcttcctatacatcctggcagaataccccccagcaaacagagagccacacccatccacacc | |
| | | gccaccaccaagcagccgctgagacggacggttccatgccagctgcctggaggaggaaca | |
| | | gacccctttagtcctcatcccttagatcctggagggcacggatcacatcctgggaagaaggca | |
| | | tctggaggataagcaaagccaccccgacacccaatcttggaagccctgagtaggcagggcc | |
| | | agggtaggtgggggccgggagggacccaggtgtgaacggatgaataaagttcaactgcaa | |
| | | ctgaaaaaaaaaaaaaaaaaaaaaaaaaaa | |
| CHTOP | NM_001206612.1 | tttcccgccctctagtcgtgcgcggatctgacgcctgacgtaattgcgtagacgccattttagcc | 6 |
| | | ggtcagacaagcactggacgtggcggccattttgttttggacaccgagcaggagctggcgg | |
| | | ccgctgcagacgaaaggcaggaaagggcaggccgggtgagcagacggatcggccgact | |
| | | agacagccaaccagcaacaacgaactgagctcgcatactaccgcttacgcatctaaccaacc | |
| | | gcccatctagctaacccgagcccctccaccgtcaactcaggttcggccggtccccggcccgc | |
| | | ctgccggagccgtggtggcagccccgggaggagcactggcgtctgtttccttcgattctcgg | |
| | | gattcgaagatggctgcacagtcagcgccgaaagttgtgctaaaaagcaccaccaagatgtc | |
| | | tctaaatgagcgctttactaatatgctgaagaacaaacagccgacgccagtgaatattcgggct | |
| | | tcgatgcagcaacaacagcagctagccagtgccagaaacagaagactggcccagcagatg | |
| | | gagaatagaccctctgtccaggcagcattaaaacttaagcagaagagcttaaagcagcgcct | |
| | | gggtaagagtaacatccaggcacggttaggccgacccataggggccctggccaggggagc | |
| | | aatcggaggacgaggcctacccataatccagagaggcttgcccagaggaggactacgtgg | |
| | | gggacgtgccaccagaaccctacttaggggcgggatgtcactccgaggtcaaaacctgctc | |
| | | cgaggtggacgagccgtagctccccgaatgggcttaagaagaggtggtgttcgaggtcgtg | |
| | | gaggtcctgggagagggggcctagggcgtggagctatgggtcgtggcggaatcggtggta | |
| | | gaggtcggggtatgataggtcggggaagagggggctttggaggccgaggccgaggccgt | |
| | | ggacgagggagaggtgcccttgctcgccctgtattgaccaaggagcagctggacaaccaat | |
| | | tggatgcatatatgtcgaaaacaaaaggacacctggatgctgagttggatgcctacatggcgc | |
| | | agacagatcccgaaaccaatgattgaagcctgcccatcctcccatgagagactcttgttagtca | |
| | | acacatctgtaaataaccttgagataacagatgagaagaaatctgattgatgctggatggacct | |
| | | atcacaataggctgtggacttacttgccaccagcttgtgcatttagtgtgttccttttactttttgata | |
| | | ctgtgttgtatgaaacccttttgtcctttgatttggttttttgtttttgttttttttaggggggagggggg | |
| | | gtttcccctcctttgcccagacttctctttgaacacaaatgcattagccttgtggctagaacaccct | |
| | | cttcctacctctgtctcccctcacttgtcatatgctctgacatgctaacatttcttttgttcatccctgt | |
| | | tgcccccacagaaacatcccagaaaaaccggtcagtgttccttcctccctgatccttaggtttct | |
| | | gaaatagggttctgttacatcctcttcgatagcctgtttaaaatgtttagaaggtctggagctcaa | |
| | | aaatgcgttcttccacattgataatttagtaaactgagaacattgacatcactacagggcagcat | |
| | | aagaggttgcttacatgtggtagcagctctggtttgattcaagttgctaccatgtacattgacagc | |
| | | acatataccataaccagcgtgttgggttgaattgcactttctacctttgtatgagatttacagacttt | |
| | | ccttctgggtttgtatcatgaccagaggggtactatagggttggtttatactgcaatatagaggat | |
| | | cagaagccatttgatttggtaggtgtgtcagaagggagaatgatggcagacgaactgctgga | |
| | | agaggtcagaagatagccatgctaaaatgcaattatatcctcatgtttatcccaaactaatcttgg | |
| | | acttttccactcattagctttgttttgcccttgtttcccttgaaggtttaagttcaaccatattctgtca | |
| | | actgttcagtttcagtggaatcttgtatttctggttcattataacaaactgttcgcttaaatccaaaa | |
| | | aaaaaa | |
| COL1A1 | NM_000088.3 | tcgtcggagcagacgggagtttctcctcggggtcggagcaggaggcacgcggagtgtgag | 7 |
| | | gccacgcatgagcggacgctaaccccctccccagccacaaagagtctacatgtctagggtct | |
| | | agacatgttcagctttgtggacctccggctcctgctcctcttagcggccaccgccctcctgacg | |
| | | cacggccaagaggaaggccaagtcgagggccaagacgaagacatcccaccaatcacctg | |
| | | cgtacagaacggcctcaggtaccatgaccgagacgtgtggaaacccgagccctgccggatc | |
| | | tgcgtctgcgacaacggcaaggtgttgtgcgatgacgtgatctgtgacgagaccaagaactg | |
| | | ccccggcgccgaagtccccgagggcgagtgctgtcccgtctgccccgacggctcagagtc | |
| | | acccaccgaccaagaaaccaccggcgtcgagggacccaagggagacactggcccccgag | |
| | | gcccaaggggacccgcaggcccccctggccgagatggcatccctggacagcctggacttc | |
| | | ccggaccccccggaccccccggacctcccggaccccctggcctcggaggaaactttgctcc | |
| | | ccagctgtcttatggctatgatgagaaatcaaccggaggaatttccgtgcctggccccatgggt | |
| | | ccctctggtcctcgtggtctccctggcccccctggtgcacctggtccccaaggcttccaaggt | |
| | | ccccctggtgagcctggcgagcctggagcttcaggtcccatgggtccccgaggtcccccag | |
| | | gtccccctggaaagaatggagatgatggggaagctggaaaacctggtcgtcctggtgagcg | |
| | | tgggcctcctgggcctcagggtgctcgaggattgcccggaacagctggcctccctggaatga | |
| | | agggacacagaggtttcagtggtttggatggtgccaagggagatgctggtcctgctggtccta | |
| | | agggtgagcctggcagccctggtgaaaatggagctcctggtcagatgggcccccgtggcct | |
| | | gcctggtgagagaggtcgccctggagcccctggccctgctggtgctcgtggaaatgatggtg | |
| | | ctactggtgctgccgggccccctggtcccaccggccccgctggtcctcctggcttccctggtg | |
| | | ctgttggtgctaagggtgaagctggtccccaagggccccgaggctctgaaggtccccaggg | |
| | | tgtgcgtggtgagcctggcccccctggccctgctggtgctgctggccctgctggaaaccctg | |
| | | gtgctgatggacagcctggtgctaaaggtgccaatggtgctcctggtattgctggtgctcctgg | |
| | | cttccctggtgcccgaggcccctctggaccccagggccccggcggccctcctggtcccaag | |
| | | ggtaacagcggtgaacctggtgctcctggcagcaaaggagacactggtgctaagggagag | |
| | | cctggccctgttggtgttcaaggaccccctggccctgctggagaggaaggaaagcgaggag | |
| | | ctcgaggtgaacccggacccactggcctgcccggaccccctggcgagcgtggtggacctg | |
| | | gtagccgtggtttccctggcgcagatggtgttgctggtcccaagggtcccgctggtgaacgtg | |
| | | gttctcctggccctgctggccccaaaggatctcctggtgaagctggtcgtcccggtgaagctg | |
| | | gtctgcctggtgccaagggtctgactggaagccctggcagccctggtcctgatggcaaaact | |
| | | ggcccccctggtcccgccggtcaagatggtcgccccggacccccaggcccacctggtgcc | |
| | | cgtggtcaggctggtgtgatgggattccctggacctaaaggtgctgctggagagcccggcaa | |
| | | ggctggagagcgaggtgttcccggaccccctggcgctgtcggtcctgctggcaaagatgga | |
| | | gaggctggagctcagggaccccctggccctgctggtcccgctggcgagagaggtgaacaa | |
| | | ggccctgctggctcccccggattccagggtctccctggtcctgctggtcctccaggtgaagca | |
| | | ggcaaacctggtgaacagggtgttcctggagaccttggcgcccctggcccctctggagcaa | |
| | | gaggcgagagaggtttccctggcgagcgtggtgtgcaaggtccccctggtcctgctggtccc | |
| | | cgaggggccaacggtgctcccggcaacgatggtgctaagggtgatgctggtgcccctgga | |
| | | gctcccggtagccagggcgcccctggccttcagggaatgcctggtgaacgtggtgcagctg | |
| | | gtcttccagggcctaagggtgacagaggtgatgctggtcccaaaggtgctgatggctctcctg | |
| | | gcaaagatggcgtccgtggtctgactggccccattggtcctcctggccctgctggtgcccctg | |
| | | gtgacaagggtgaaagtggtcccagcggccctgctggtcccactggagctcgtggtgcccc | |
| | | cggagaccgtggtgagcctggtccccccggccctgctggctttgctggcccccctggtgctg | |
| | | acggccaacctggtgctaaaggcgaacctggtgatgctggtgctaaaggcgatgctggtccc | |
| | | cctggccctgccggacccgctggaccccctggccccattggtaatgttggtgctcctggagc | |
| | | caaaggtgctcgcggcagcgctggtccccctggtgctactggtttccctggtgctgctggccg | |
| | | agtcggtcctcctggcccctctggaaatgctggaccccctggccctcctggtcctgctggcaa | |
| | | agaaggcggcaaaggtccccgtggtgagactggccctgctggacgtcctggtgaagttggt | |
| | | ccccctggtccccctggccctgctggcgagaaaggatcccctggtgctgatggtcctgctggt | |
| | | gctcctggtactcccgggcctcaaggtattgctggacagcgtggtgtggtcggcctgcctggt | |
| | | cagagaggagagagaggcttccctggtcttcctggcccctctggtgaacctggcaaacaag | |
| | | gtccctctggagcaagtggtgaacgtggtccccctggtcccatgggcccccctggattggct | |
| | | ggaccccctggtgaatctggacgtgagggggctcctggtgccgaaggttcccctggacgag | |
| | | acggttctcctggcgccaagggtgaccgtggtgagaccggccccgctggaccccctggtgc | |
| | | tcctggtgctcctggtgcccctggccccgttggccctgctggcaagagtggtgatcgtggtga | |
| | | gactggtcctgctggtcccgccggtcctgtcggccctgttggcgcccgtggccccgccggac | |
| | | cccaaggcccccgtggtgacaagggtgagacaggcgaacagggcgacagaggcataaag | |
| | | ggtcaccgtggcttctctggcctccagggtccccctggccctcctggctctcctggtgaacaa | |
| | | ggtccctctggagcctctggtcctgctggtccccgaggtccccctggctctgctggtgctcctg | |
| | | gcaaagatggactcaacggtctccctggccccattgggccccctggtcctcgcggtcgcact | |
| | | ggtgatgctggtcctgttggtccccccggccctcctggacctcctggtccccctggtcctccca | |
| | | gcgctggtttcgacttcagcttcctgccccagccacctcaagagaaggctcacgatggtggcc | |
| | | gctactaccgggctgatgatgccaatgtggttcgtgaccgtgacctcgaggtggacaccacc | |
| | | ctcaagagcctgagccagcagatcgagaacatccggagcccagagggcagccgcaagaa | |
| | | ccccgcccgcacctgccgtgacctcaagatgtgccactctgactggaagagtggagagtact | |
| | | ggattgaccccaaccaaggctgcaacctggatgccatcaaagtcttctgcaacatggagactg | |
| | | gtgagacctgcgtgtaccccactcagcccagtgtggcccagaagaactggtacatcagcaag | |
| | | aaccccaaggacaagaggcatgtctggttcggcgagagcatgaccgatggattccagttcga | |
| | | gtatggcggccagggctccgaccctgccgatgtggccatccagctgaccttcctgcgcctga | |
| | | tgtccaccgaggcctcccagaacatcacctaccactgcaagaacagcgtggcctacatggac | |
| | | cagcagactggcaacctcaagaaggccctgctcctccagggctccaacgagatcgagatcc | |
| | | gcgccgagggcaacagccgcttcacctacagcgtcactgtcgatggctgcacgagtcacac | |
| | | cggagcctggggcaagacagtgattgaatacaaaaccaccaagacctcccgcctgcccatc | |
| | | atcgatgtggcccccttggacgttggtgccccagaccaggaattcggcttcgacgttggccct | |
| | | gtctgcttcctgtaaactccctccatcccaacctggctccctcccacccaaccaactttcccccc | |
| | | aacccggaaacagacaagcaacccaaactgaaccccctcaaaagccaaaaaatgggagac | |
| | | aatttcacatggactttggaaaatatttttttcctttgcattcatctctcaaacttagtttttatctttgac | |
| | | caaccgaacatgaccaaaaaccaaaagtgcattcaaccttaccaaaaaaaaaaaaaaaaaaa | |
| | | gaataaataaataactttttaaaaaaggaagcttggtccacttgcttgaagacccatgcggggg | |
| | | taagtccctttctgcccgttgggcttatgaaaccccaatgctgccctttctgctcctttctccacac | |
| | | cccccttggggcctcccctccactccttcccaaatctgtctccccagaagacacaggaaacaa | |
| | | tgtattgtctgcccagcaatcaaaggcaatgctcaaacacccaagtggcccccaccctcagcc | |
| | | cgctcctgcccgcccagcacccccaggccctgggggacctggggttctcagactgccaaag | |
| | | aagccttgccatctggcgctcccatggctcttgcaacatctccccttcgtttttgagggggtcat | |
| | | gccgggggagccaccagcccctcactgggttcggaggagagtcaggaagggccacgaca | |
| | | aagcagaaacatcggatttggggaacgcgtgtcaatcccttgtgccgcagggctgggcggg | |
| | | agagactgttctgttccttgtgtaactgtgttgctgaaagactacctcgttcttgtcttgatgtgtca | |
| | | ccggggcaactgcctgggggcggggatgggggcagggtggaagcggctccccattttata | |
| | | ccaaaggtgctacatctatgtgatgggtggggtggggagggaatcactggtgctatagaaatt | |
| | | gagatgcccccccaggccagcaaatgttcctttttgttcaaagtctatttttattccttgatatttttc | |
| | | tttttttttttttttttttgtggatggggacttgtgaatttttctaaaggtgctatttaacatgggaggag | |
| | | agcgtgtgcggctccagcccagcccgctgctcactttccaccctctctccacctgcctctggct | |
| | | tctcaggcctctgctctccgacctctctcctctgaaaccctcctccacagctgcagcccatcctc | |
| | | ccggctccctcctagtctgtcctgcgtcctctgtccccgggtttcagagacaacttcccaaagc | |
| | | acaaagcagtttttccccctaggggtgggaggaagcaaaagactctgtacctattttgtatgtgt | |
| | | ataataatttgagatgtttttaattattttgattgctggaataaagcatgtggaaatgacccaaacat | |
| | | aa | |
| EDC4 | NM_014329.4 | tttcttctccggccctggcaggttccgggagcctcggctcgtgggtgccggaagtggaggcg | 8 |
| | | gttggtggggttggcggggctcagcgacgctgcgcgggtggcggtttgcgaactgcgggtg | |
| | | gactgtgtagtgaccggcgtcccgctgtctcgccccgtggcgggtgagcgagggtgcgtgg | |
| | | tgcgcggcggcggcggaacgaacgcggtgcgggcggggcgcccgccgcagggcccat | |
| | | ggcctcctgcgcgagcatcgacatcgaggacgccacgcagcacctgcgggacatcctcaa | |
| | | gctggaccggcccgcgggcggccccagtgcagagagcccacggccatccagtgcctaca | |
| | | atggggacctcaatggacttctggtcccagacccgctctgctcaggtgatagtacctcagcaa | |
| | | acaagactggtcttcggaccatgccacccattaacctgcaagagaagcaggtcatctgtctctc | |
| | | aggagatgatagctccacctgcattgggattttggccaaggaggtggagattgtggctagcag | |
| | | tgactctagcatttcaagcaaggcccggggaagcaacaaggtgaaaattcagcctgtcgcca | |
| | | agtatgactgggaacagaagtactactatggcaacctgattgctgtgtctaactccttcttggcc | |
| | | tatgccattcgggctgccaacaatggctctgccatggtgcgggtgatcagcgtcagcacttcg | |
| | | gagcggaccttgctcaagggcttcacaggcagtgtggctgatctggctttcgcgcacctcaac | |
| | | tctccacagctggcctgcctggatgaggcaggcaacctgttcgtgtggcgcttggctctggtta | |
| | | atggcaaaattcaagaagagatcttggtccatattcggcagccagagggcacgccactgaac | |
| | | cactttcgcaggatcatctggtgccccttcatccctgaggagagcgaagactgctgtgaggag | |
| | | agcagcccaacagtggccctgctgcatgaagaccgggctgaggtgtgggacctggacatg | |
| | | ctccgctccagccacagtacctggcctgtggatgttagccagatcaagcagggcttcattgtg | |
| | | gtaaaaggtcatagcacgtgcctcagtgaaggagccctctctcctgatgggactgtgctggct | |
| | | actgcgagccacgatggctatgtcaagttctggcagatctacattgaggggcaagatgagcc | |
| | | aaggtgtctgcacgagtggaaacctcatgatgggcggcccctctcctgcctcctgttctgtgac | |
| | | aaccataagaaacaagaccctgatgtccctttctggaggttccttattactggtgctgaccagaa | |
| | | ccgagagttaaagatgtggtgtacagtatcctggacctgcctgcagactattcgcttctcccca | |
| | | gatatcttcagctcagtgagtgtgccccctagcctcaaggtttgcttggacctctcagcagaata | |
| | | cctgattctcagcgatgtgcaacggaaggtcctctatgtgatggagctgctgcaaaaccagga | |
| | | ggagggccacgcctgcttcagctccatctcggagttcctgctcacccaccctgtgctgagcttt | |
| | | ggtatccaggttgtgagtcgctgccggctacggcacactgaggtgctgcctgccgaagagg | |
| | | aaaatgacagcctgggtgctgatggtacccatggagccggtgccatggagtctgcggccgg | |
| | | tgtgctcatcaagctcttttgtgtgcatactaaggcactgcaagatgtgcagatccgcttccagc | |
| | | cacagctgaaccctgatgtggtggccccactgcccacccacactgcccacgaggacttcaca | |
| | | tttggagagtctcggcccgaactgggctctgagggcctggggtcagccgctcacggctccca | |
| | | gcctgacctccgacgaatcgtggagctgcctgcacctgccgacttcctcagtctgagcagtga | |
| | | gaccaagcccaagttgatgacacctgacgccttcatgacacctagcgcctccttgcagcagat | |
| | | cactgcctctcccagcagcagcagcagcggtagcagcagcagcagcagcagtagcagcag | |
| | | ctcccttacagctgtgtctgccatgagcagcacctcagctgtggacccctccttgaccaggcc | |
| | | acctgaggagctgaccttgagccccaagctgcagctggatggcagcctgacaatgagcagc | |
| | | agtggcagccttcaggcaagcccgcgtggcctcctgcctggcctgctcccagccccagctg | |
| | | acaaactgactcccaaggggccgggccaggtgcctactgccacctctgcactgtccctggag | |
| | | ctgcaggaagtggagcccctggggctaccccaagcctcccctagccgcactcgttcccctga | |
| | | tgtcatctcctcagcttccactgccctgtcccaggacatccctgagattgcatctgaggccctgt | |
| | | cccgtggttttggctcctctgcaccagagggccttgagccagacagtatggcttcagccgcct | |
| | | cggcactgcacctgctgtccccacggccccggccagggcccgagctcggcccccagctcg | |
| | | ggcttgatggaggccctggggatggagatcggcataataccccctccctcctggaggcagc | |
| | | cttgacccaggaggcctcgactcctgacagtcaggtttggcccacagcacctgacattactcg | |
| | | tgagacctgcagcaccctggcagaaagccccaggaatggccttcaggaaaagcacaagag | |
| | | cctggccttccaccgaccaccatatcacctgctgcagcaacgtgacagccaggatgccagtg | |
| | | ctgagcaaagtgaccatgatgatgaggtggccagccttgcctctgcttcaggaggctttggca | |
| | | ccaaagttcctgctccacggctgcctgccaaggactggaagaccaagggatcccctcgaac | |
| | | ctcacccaagctcaagaggaaaagcaagaaggatgatggggatgcagccatgggatcccg | |
| | | gctcacagagcaccaggtggcagagccccctgaggactggccagcactaatttggcaacag | |
| | | cagagagagctggcagagctgcggcacagccaggaagagctgctgcagcgtctgtgtacc | |
| | | caactcgaaggcctgcagagcacagtcacaggccacgtagaacgtgcccttgagactcggc | |
| | | acgagcaggaacagcggcggctggagcgagcactggctgaggggcagcagcggggag | |
| | | ggcagctgcaggagcagctgacacaacagttgtcccaagcactgtcgtcagctgtagctgg | |
| | | gcggctagagcgcagcatacgggatgagatcaagaagacagtccctccatgtgtctcaagg | |
| | | agtctggagcctatggcaggccaactgagcaactcagtggctaccaagctcacagctgtgga | |
| | | gggcagcatgaaagagaacatctccaagctgctcaagtccaagaacttgactgatgccatcg | |
| | | cccgagcagctgcagacacattacaagggccgatgcaggctgcctaccgggaagccttcca | |
| | | gagtgtggtgctgccggcctttgagaagagctgccaggccatgttccagcaaatcaatgatag | |
| | | cttccggctggggacacaggaatacttgcagcagctagaaagccacatgaagagccggaag | |
| | | gcacgggaacaggaggccagggagcctgtgctagcccagctgcggggcctggtcagcac | |
| | | actgcagagtgccactgagcagatggcagccaccgtggccggcagtgttcgtgctgaggtg | |
| | | cagcaccagctgcatgtggctgtgggcagcctgcaggagtccattttagcacaggtacagcg | |
| | | catcgttaagggtgaggtgagtgtggcgctcaaggagcagcaggccgccgtcacctccagc | |
| | | atcatgcaggccatgcgctcagctgctggcacacctgtcccctctgcccaccttgactgccag | |
| | | gcccagcaagcccatatcctgcagctgctgcagcagggccacctcaatcaggccttccagca | |
| | | ggcgctgacagctgctgacctgaacctggtgctgtatgtgtgtgaaactgtggacccagccca | |
| | | ggtttttgggcagccaccctgcccgctctcccagcctgtgctcctttccctcatccagcagctg | |
| | | gcatctgaccttggcactcgaactgacctcaagctcagctacctggaagaggccgtgatgca | |
| | | cctggaccacagtgaccccatcactcgggaccacatgggctccgttatggcccaggtgcgcc | |
| | | aaaagctttttcagttcctgcaggctgagccacacaactcacttggcaaagcagctcggcgtct | |
| | | cagcctcatgctgcatggcctcgtgacccccagcctcccttagctgctaagcctgccttgccca | |
| | | ggggtgggatggcactgaaggccagcagacaggcctaggctggggcagggtcacggctg | |
| | | gcctttacctgctcaggcccccatctctggggtgtttgggggtcagggagcagggagcactg | |
| | | gccgtggtctacagcgtgtggtagtcagaaggtttagctgggcccagggcaggtattgcgcc | |
| | | tgcttgggttctgccatgcctggagcatgaccctgagatcgtgacaccacttgagtggaattttc | |
| | | catgttcctttttacctctaatttggatctttttgtttttgaaaaacattgagaaattcaattaaatgcttt | |
| | | tggaataaaatggagtatgtgtgtg | |
| FGFR2 | NM_000141.4 | ggcggcggctggaggagagcgcggtggagagccgagcgggcgggcggcgggtgcgga | 9 |
| | | gcgggcgagggagcgcgcgcggccgccacaaagctcgggcgccgcggggctgcatgc | |
| | | ggcgtacctggcccggcgcggcgactgctctccgggctggcgggggccggccgcgagcc | |
| | | ccgggggccccgaggccgcagcttgcctgcgcgctctgagccttcgcaactcgcgagcaa | |
| | | agtttggtggaggcaacgccaagcctgagtcctttcttcctctcgttccccaaatccgagggca | |
| | | gcccgcgggcgtcatgcccgcgctcctccgcagcctggggtacgcgtgaagcccgggag | |
| | | gcttggcgccggcgaagacccaaggaccactcttctgcgtttggagttgctccccgcaaccc | |
| | | cgggctcgtcgctttctccatcccgacccacgcggggcgcggggacaacacaggtcgcgg | |
| | | aggagcgttgccattcaagtgactgcagcagcagcggcagcgcctcggttcctgagcccac | |
| | | cgcaggctgaaggcattgcgcgtagtccatgcccgtagaggaagtgtgcagatgggattaac | |
| | | gtccacatggagatatggaagaggaccggggattggtaccgtaaccatggtcagctggggt | |
| | | cgtttcatctgcctggtcgtggtcaccatggcaaccttgtccctggcccggccctccttcagttt | |
| | | agttgaggataccacattagagccagaagagccaccaaccaaataccaaatctctcaaccag | |
| | | aagtgtacgtggctgcgccaggggagtcgctagaggtgcgctgcctgttgaaagatgccgc | |
| | | cgtgatcagttggactaaggatggggtgcacttggggcccaacaataggacagtgcttattgg | |
| | | ggagtacttgcagataaagggcgccacgcctagagactccggcctctatgcttgtactgcca | |
| | | gtaggactgtagacagtgaaacttggtacttcatggtgaatgtcacagatgccatctcatccgg | |
| | | agatgatgaggatgacaccgatggtgcggaagattttgtcagtgagaacagtaacaacaaga | |
| | | gagcaccatactggaccaacacagaaaagatggaaaagcggctccatgctgtgcctgcggc | |
| | | caacactgtcaagtttcgctgcccagccggggggaacccaatgccaaccatgcggtggctg | |
| | | aaaaacgggaaggagtttaagcaggagcatcgcattggaggctacaaggtacgaaaccagc | |
| | | actggagcctcattatggaaagtgtggtcccatctgacaagggaaattatacctgtgtagtgga | |
| | | gaatgaatacgggtccatcaatcacacgtaccacctggatgttgtggagcgatcgcctcaccg | |
| | | gcccatcctccaagccggactgccggcaaatgcctccacagtggtcggaggagacgtagag | |
| | | tttgtctgcaaggtttacagtgatgcccagccccacatccagtggatcaagcacgtggaaaag | |
| | | aacggcagtaaatacgggcccgacgggctgccctacctcaaggttctcaaggccgccggtg | |
| | | ttaacaccacggacaaagagattgaggttctctatattcggaatgtaacttttgaggacgctgg | |
| | | ggaatatacgtgcttggcgggtaattctattgggatatcctttcactctgcatggttgacagttctg | |
| | | ccagcgcctggaagagaaaaggagattacagcttccccagactacctggagatagccattta | |
| | | ctgcataggggtcttcttaatcgcctgtatggtggtaacagtcatcctgtgccgaatgaagaac | |
| | | acgaccaagaagccagacttcagcagccagccggctgtgcacaagctgaccaaacgtatcc | |
| | | ccctgcggagacaggtaacagtttcggctgagtccagctcctccatgaactccaacaccccg | |
| | | ctggtgaggataacaacacgcctctcttcaacggcagacacccccatgctggcaggggtctc | |
| | | cgagtatgaacttccagaggacccaaaatgggagtttccaagagataagctgacactgggca | |
| | | agcccctgggagaaggttgctttgggcaagtggtcatggcggaagcagtgggaattgacaa | |
| | | agacaagcccaaggaggcggtcaccgtggccgtgaagatgttgaaagatgatgccacaga | |
| | | gaaagacctttctgatctggtgtcagagatggagatgatgaagatgattgggaaacacaagaa | |
| | | tatcataaatcttcttggagcctgcacacaggatgggcctctctatgtcatagttgagtatgcctc | |
| | | taaaggcaacctccgagaatacctccgagcccggaggccacccgggatggagtactcctat | |
| | | gacattaaccgtgttcctgaggagcagatgaccttcaaggacttggtgtcatgcacctaccag | |
| | | ctggccagaggcatggagtacttggcttcccaaaaatgtattcatcgagatttagcagccaga | |
| | | aatgttttggtaacagaaaacaatgtgatgaaaatagcagactttggactcgccagagatatca | |
| | | acaatatagactattacaaaaagaccaccaatgggcggcttccagtcaagtggatggctccag | |
| | | aagccctgtttgatagagtatacactcatcagagtgatgtctggtccttcggggtgttaatgtgg | |
| | | gagatcttcactttagggggctcgccctacccagggattcccgtggaggaactttttaagctgc | |
| | | tgaaggaaggacacagaatggataagccagccaactgcaccaacgaactgtacatgatgat | |
| | | gagggactgttggcatgcagtgccctcccagagaccaacgttcaagcagttggtagaagact | |
| | | tggatcgaattctcactctcacaaccaatgaggaatacttggacctcagccaacctctcgaaca | |
| | | gtattcacctagttaccctgacacaagaagttcttgttcttcaggagatgattctgttttttctccag | |
| | | accccatgccttacgaaccatgccttcctcagtatccacacataaacggcagtgttaaaacatg | |
| | | aatgactgtgtctgcctgtccccaaacaggacagcactgggaacctagctacactgagcagg | |
| | | gagaccatgcctcccagagcttgttgtctccacttgtatatatggatcagaggagtaaataattg | |
| | | gaaaagtaatcagcatatgtgtaaagatttatacagttgaaaacttgtaatcttccccaggagga | |
| | | gaagaaggtttctggagcagtggactgccacaagccaccatgtaacccctctcacctgccgt | |
| | | gcgtactggctgtggaccagtaggactcaaggtggacgtgcgttctgccttccttgttaattttgt | |
| | | aataattggagaagatttatgtcagcacacacttacagagcacaaatgcagtatataggtgctg | |
| | | gatgtatgtaaatatattcaaattatgtataaatatatattatatatttacaaggagttattttttgtatt | |
| | | gattttaaatggatgtcccaatgcacctagaaaattggtctctctttttttaatagctatttgctaaat | |
| | | gctgttcttacacataatttcttaattttcaccgagcagaggtggaaaaatacttttgctttcaggg | |
| | | aaaatggtataacgttaatttattaataaattggtaatatacaaaacaattaatcatttatagtttttttt | |
| | | gtaatttaagtggcatttctatgcaggcagcacagcagactagttaatctattgcttggacttaac | |
| | | tagttatcagatcctttgaaaagagaatatttacaatatatgactaatttggggaaaatgaagtttt | |
| | | gatttatttgtgtttaaatgctgctgtcagacgattgttcttagacctcctaaatgccccatattaaa | |
| | | agaactcattcataggaaggtgtttcattttggtgtgcaaccctgtcattacgtcaacgcaacgtc | |
| | | taactggacttcccaagataaatggtaccagcgtcctcttaaaagatgccttaatccattccttga | |
| | | ggacagaccttagttgaaatgatagcagaatgtgcttctctctggcagctggccttctgcttctg | |
| | | agttgcacattaatcagattagcctgtattctcttcagtgaattttgataatggcttccagactcttt | |
| | | ggcgttggagacgcctgttaggatcttcaagtcccatcatagaaaattgaaacacagagttgtt | |
| | | ctgctgatagttttggggatacgtccatctttttaagggattgctttcatctaattctggcaggacct | |
| | | caccaaaagatccagcctcatacctacatcagacaaaatatcgccgttgttccttctgtactaaa | |
| | | gtattgtgttttgctttggaaacacccactcactttgcaatagccgtgcaagatgaatgcagatta | |
| | | cactgatcttatgtgttacaaaattggagaaagtatttaataaaacctgttaatttttatactgacaa | |
| | | taaaaatgtttctacagatattaatgttaacaagacaaaataaatgtcacgcaacttatttttttaata | |
| | | aaaaaaaaaaaaaa | |
| FXYD7 | NM_022006.1 | ccccacatcggtccgtcctgcttccagctgctgcagcgcgccttcgccgccaaagcatccag | 10 |
| | | cagccccctgctccggcccagcatggcgaccccgacccagacccccacaaaggctcctga | |
| | | ggaacctgacccattttactatgactacaacacggtgcagactgtgggcatgactctggcaac | |
| | | catcttgttcctgctgggtatcctcatcgtcatcagcaagaaggtgaagtgcaggaaggcgga | |
| | | ctccaggtctgagagcccaacctgcaaatcctgtaagtctgagcttccctcttcagcccctggt | |
| | | ggcggcggcgtgtaacaccttcccgaggaaactccgctgccgaccctgcctgagcgcggg | |
| | | agcctgaggaccgggtggaggcggtggggacccagccgcgcgccgggagcgctccccg | |
| | | gaatgagccgccccacccaccccaaggctggagccgctgcaccctgctgtccctctccagg | |
| | | ccttggcaatgacgatcccccaaagagcccgtctgcaccccagacccagggcctcaggcct | |
| | | ccagctcctgggatccgggagtccatcccggcccagcacccccagcatccccgtgtatggc | |
| | | ccccctgcacctccttgtctcatccccgaagatccgtccccctggcccctcagtgtccatgtctt | |
| | | gagcttaataaatgtgcatttggttttttcctctg | |
| FYCO1 | NM_024513.3 | accctcttggtgtgcgcgtcatggccgtcagcaccgcgttcccgtcctcttccgcttggcccca | 11 |
| | | gaaagtttcggttctgcccggcggtggacccacgagcgcgtgccaccatggagtctgacca | |
| | | ctgctgagcagacagccaccgagggccgaaattctgagccttcctctggacccaggcagga | |
| | | gacatacagacaagaaaggcaaactcaccatggcctccaccaatgcagagagccagctcca | |
| | | gagaatcatccgagacttgcaagatgctgtgacagaactaagcaaagaatttcaggaagcag | |
| | | gggaacccatcacggatgacagcaccagcttgcataaattttcttataaacttgagtatctcctg | |
| | | caatttgatcagaaagagaaggccaccctcctgggcaacaagaaggactactgggattacttc | |
| | | tgtgcctgcctggccaaggtgaaaggagccaatgatgggatccgctttgtcaagtctatctca | |
| | | gagctccgaacatccttggggaaaggaagagcatttattcgctactccttggtgcaccagagg | |
| | | ttggcagacaccttacagcagtgcttcatgaacaccaaagtgaccagtgactggtactatgca | |
| | | agaagcccctttctgcagccaaagctgagctcggacattgtgggccaactctatgagctgact | |
| | | gaggttcagtttgacctggcgtcgaggggctttgacttggatgctgcctggccaacatttgcca | |
| | | ggaggacgctgaccactggctcttctgcttacctgtggaaaccccctagccgcagctccagc | |
| | | atgagcagcttggtgagcagctacctgcagactcaagagatggtgtccaactttgacctgaac | |
| | | agccccctaaacaacgaggcattggagggctttgatgagatgcgactagagctggaccagtt | |
| | | ggaggtgcgggagaagcagctacgggagcgcatgcagcagctggacagagagaaccag | |
| | | gagctgagggcagctgtcagccagcaaggggagcaactgcagacagagagggagaggg | |
| | | ggcgcactgcagcggaggacaacgttcgcctcacttgcttggtagctgagctccagaagca | |
| | | gtgggaggtcacccaggccacccagaacactgtgaaggagctgcagacatgcctgcaggg | |
| | | cctggagctaggagcagcagagaaggaggaggactaccacacagccctgcggcggctgg | |
| | | agtccatgctgcagcccttggcacaggagcttgaggccacacgggactcactggacaagaa | |
| | | aaaccagcatttagccagcttcccaggctggctagccatggctcagcagaaggcagatacgg | |
| | | catcagacacaaagggccggcaagaacctattcccagtgatgcggcccaggagatgcagg | |
| | | agctaggggagaagcttcaagccctagaaagggagagaaccaaggtcgaggaggtcaac | |
| | | agacagcagagtgcccaactggaacagctggtcaaggagcttcagctgaaagaggatgccc | |
| | | gggccagcctggagcgcctggtgaaggagatggccccactccaggaggagttgtctggga | |
| | | agggacaggaggcagaccagctctggcgacggctgcaggagttgctggcccacacgagct | |
| | | cctgggaggaggagctagcagagttgaggcgggagaaaaaacagcaacaggaggagaa | |
| | | ggagctgctggagcaggaggtcaggtctctgacccggcagctgcagttcctggagacccag | |
| | | ctggcacaggtgagccaacatgtgagtgacctggaggagcagaagaagcagctcattcagg | |
| | | acaaagaccacctcagccagcaggtgggtatgctcgagcggcttgctgggccgcctggccc | |
| | | agaactgccagtggcaggtgagaagaatgaggccctggtccctgtgaactccagtctgcaa | |
| | | gaggcctgggggaagccagaggaggagcagaggggcctgcaggaggcacagttagacg | |
| | | ataccaaggtgcaagagggcagccaggaggaagagctccggcaggccaacagggagct | |
| | | ggagaaggagctacagaatgtggtcgggcgtaaccagctcctggagggcaagctgcaagc | |
| | | cctgcaggccgattaccaggctttgcagcagcgggaatcagccatccagggctccttggcct | |
| | | ccctggaggccgagcaggccagcatccggcacttgggtgaccagatggaggcgagcttgc | |
| | | tggctgtaaggaaggccaaggaggccatgaaagcccagatggcagagaaggaggccattc | |
| | | tacagagcaaggagggcgagtgtcagcagctgcgggaggaggtggagcagtgccagcaa | |
| | | ctggcagaagcccggcacagagagcttagggctctcgagagccagtgccagcagcagacc | |
| | | cagctgattgaggtcctcacagcagagaaaggccaacagggagttggcccacccactgaca | |
| | | atgaagcccgtgagctggctgcccagctagccctgtctcaggcgcagctggaagtccatcag | |
| | | ggggaggtccaacggctgcaggctcaggtggtggacctccaggccaagatgcgggcagc | |
| | | cctggatgaccaggacaaggtgcagagccagctaagcatggctgaggccgtcctgaggga | |
| | | gcacaaaacccttgtgcagcagctgaaggagcagaatgaagcccttaacagagcccatgtc | |
| | | caggagctgctgcaatgctcggagcgtgaaggggcactgcaggaggagagggccgatga | |
| | | ggcccagcagagggaggaggagctgcgggccctgcaggaggagctgtcccaggccaaat | |
| | | gcagctccgaggaagcacagctggagcacgctgagctgcaagagcagctgcaccgggcc | |
| | | aacacagacacagctgagctgggcatccaggtttgcgcactgaccgtggaaaaggagcga | |
| | | gtggaggaggcactggcctgtgctgtccaggagctccaggacgccaaagaggcagcctca | |
| | | agggagcgagagggcctggagcgccaagtagctgggctgcagcaagagaaggagagctt | |
| | | gcaggagaagctgaaggcggccaaggcagcagccggctcactgcctggcctgcaggccc | |
| | | agctcgcccaggcagagcagcgggcccagagcctccaagaggctgcacaccaggagctc | |
| | | aacaccctcaagttccagctgagtgctgaaatcatggactaccagagcagacttaagaatgct | |
| | | ggtgaagagtgcaagagcctcaggggccagcttgaggagcaaggccggcagctgcaggc | |
| | | tgctgaggaagctgtggagaagctgaaggccacccaagcagacatgggagagaagctgag | |
| | | ctgcactagcaaccatcttgcagagtgccaggcggccatgctgaggaaggacaaggaggg | |
| | | ggctgccctgcgtgaagacctagaaaggacccagaaggaactcgaaaaagccacaacaaa | |
| | | aatccaagagtattacaacaaactctgccaggaggtgacaaatcgtgagaggaatgaccaga | |
| | | agatgcttgctgacctggatgacctcaacagaaccaagaagtatctcgaggagcggctgata | |
| | | gagctgctcagggacaaggatgctctctggcagaagtcagatgccctggaattccagcagaa | |
| | | gctcagtgctgaggagagatggctcggagacacagaggcaaaccactgcctcgactgtaag | |
| | | cgggagttcagctggatggtgcggcggcaccactgcaggatatgtggccgcatcttctgttac | |
| | | tactgctgcaacaactacgtcctgagcaagcacggtggcaaaaaggagcgctgctgccgag | |
| | | cctgtttccagaagctcagtgaaggccctggctcccctgatagcagtggctcaggcactagcc | |
| | | agggagagcccagccctgcactgtcaccagcctcacctgggccccaggccacaggaggcc | |
| | | aaggagcaaatacagactacaggccaccggacgacgctgtgtttgatatcatcacagatgag | |
| | | gaattgtgccagatacaggagtccggctcctctttgcctgaaacacccactgaaactgattctct | |
| | | cgacccaaatgcggctgaacaggatactacatcaacctcgctaacgcctgaggacactgaag | |
| | | acatgcccgtggggcaggattcggaaatctgcctgctgaagtctggagaactgatgatcaaa | |
| | | gtacccctcacagtggatgagatcgccagcttcggggagggtagcagggagctgtttgtgag | |
| | | gtccagcacctacagcctgatccccatcactgtggccgaggcaggcctcaccatcagctggg | |
| | | tcttctcctctgaccccaagagcatctccttcagtgtggtcttccaggaggccgaggacacacc | |
| | | gctggatcagtgtaaggtcctcattcccacgacccgatgcaactcccacaaggagaacatcc | |
| | | agggccagctcaaggttcgcacacccggcatctacatgctcatcttcgacaataccttctcaag | |
| | | gtttgtctctaaaaaggtattttatcacttgacggttgatcggcctgtgatctacgatggaagtgat | |
| | | ttcctgtagcttcagcacctcagtaacttcacttcatccacaggaaacactgctcttcctcacctg | |
| | | tcacataaagcatttttttaaaaagtcagctgctccaaaatcatcaactcagcccctgggctgcc | |
| | | cctcagaggcggtgtctggggaggactttgtgctcagcactctgcaccggccactcttagccc | |
| | | ccgaggcgttgaagggctcaggcaatgtttccattaagtagagactcagctgttgtcacaccc | |
| | | aaagggatgctctgccaaaggtttaaacacccaggagaccatcagcctctcctgggagcaca | |
| | | gttggctacaggcctcttgtggagagtttcacgggcaggggtgattccaacttctgcctgtgga | |
| | | gagattttctgccctgccccaccagggccctgcatgttggagactgagctgggtgcactggcc | |
| | | atgccctgtgaatcctcaggctgtgacgccctcaggtactcctgggaaaaggaggtacacag | |
| | | ccatcatgcgagtcggtgccaggggaccccccggagatcctgaccagctcctccagtcatgc | |
| | | tcttgtccctcactgccccagtaagctggaggctgctccagaactcagcagtgttggaggggc | |
| | | ctctaagctgcactctctttctggcccttttgtctgggtgattctgtcctcaaataaagcccttcact | |
| | | cagccagacctctccacagctcaaagcattgccctaagaatcagaagtaaagataatccaag | |
| | | agcaaaacccactgtacttggggcctgcaatggctgtgtgtacactacatctaatgcccaaatg | |
| | | ccagccagtgtggatgttgtgaccacagagcaggattgtgcattggctttagagctactcctca | |
| | | gctgatggcccacttttgtttatataaataagagcttctgccccacctgcagacatgtttactaatg | |
| | | atcatagccaggattagaaccactttcaaacattggggccttcttaacaaaagtctttgataactt | |
| | | aagaaccaaagtaacagagtaaacagaggcatgatggatccctgggccccactcccctcctg | |
| | | acaggttccccaacagcccatttgcccacttcccactgctcagcccacaccagacctccagga | |
| | | gacatccccccttgaggcagagagatcctgttccctattcccagacaagaattatttaatcttcc | |
| | | ctgttctctgtggtccttttcttccccaacaacagatagctcaccttggacagctcttcgtcccttg | |
| | | ttcatggaaccagctgcctgcagtcaggccccaggttcttccatgggtgaacagagcatctga | |
| | | caaaaggtcccagtttggccaggggtgagggagagagcaccagacaggctatccgagaat | |
| | | ctgagagctgggcccggcagttcctccagctacccttgtgacctaagtccagtcacacatttcc | |
| | | caaagtttctctttgtcataaccctggtctggctggttttgagggcttgagaatgggtcagggact | |
| | | ccaggccaagtccaacagagaccccaaacccaccacacaccagcagccacaacctcacca | |
| | | ccaacaaagaggacttttgtggggccacaagtaagaggtcatttctggaatggactcagacct | |
| | | ttaaacaggagagttgagcacttccagtcagtttttaagcaaggcatggggaacagggaatag | |
| | | aacctttcaaagaggttgcccagagaaaagctgggcctcttgcattcggcttccttggagcag | |
| | | cctcttctggcagaaagccatcaggtgctcaatcatcttctcctggccaaggctctgaccatgct | |
| | | tagtactggaatagaggtggccaggcccccagcgactcttcttggcctgatgtttgtcctcaca | |
| | | ggcatgccacgtggcctgagatgattcagaacaaatcatgctaactttgaatccatccagccac | |
| | | ttgcaaatgataatcagaagtcagcttgttcactgttagaaagaaactaacaaaagagaaccca | |
| | | gagcaatctagaatctttgagtgcttggctttccaaggatactgcggagactctggccaagctg | |
| | | atgaccttctgaagtgtcactggcaccatatgcaacaagaaccaccattcactgagtagctaat | |
| | | gggtttggggcctgggacattccatctgaggtccttcctgaacatgtcactccacagcagagg | |
| | | accggttgcagcttacccagaaccactcctccaggagagctggatgttttgcgtgcaacacctt | |
| | | gagcactgactgctattgttcaaaaaaagcctttgctgcattcggaggactgccccgtgccctg | |
| | | aggtgacttcctaactatgtggtttcattagcgaatttattttttgtgctgggtggacatttgtattttg | |
| | | ttaggttgctgtttaagctcaagtttgctgtgctctctgcagctacaaaacatcttggcatatttaa | |
| | | gagtggcttttataaatagctttattctgatattaatcagattcccaactttactgagaattaaggac | |
| | | tggggtactttaaagaaatgcaaatagcaattgaagaaccactgctgcaggtggtagccctgg | |
| | | ctagactgaattacactagaaatcagccagaaggaagcgtccttgggatcccagatcactcttt | |
| | | ttttttttttttaaaaggggcagccccttgatggctcatctctctgaataacagttacgtcttcatatc | |
| | | gataccagatgccttcttcatcatgccactgaagccactcaccaccttcaagaacatgccaacc | |
| | | tctgtcagattcacttacccacaaacaaggaggcacgtttggcacaaagtgttgtcctccaggt | |
| | | ccaagtggactctacagagtgcttgacctcaacacactggattccaggtggactggaccaag | |
| | | agcaggcaaagacacgggaactgaaaaactccacagggtttggagaatagaaatgaaaag | |
| | | ccacgtcatataactcaagaataaatggtgttttggaaattttaaaattatcatcgaaggtggtga | |
| | | aactatttcaggcccaaatgaaaggaaatcgccagttggggatgaaatcacagagcctgtgtt | |
| | | ttatgatatggttggatgtccactgatgaaattttaaaggagtttcagttttaaaagtgcgcatgatt | |
| | | ctacatatgagaattctttaggccaagaaactgtccttggctcagaggtgttgggaattaaagca | |
| | | gagagaagccattcgtgatgcttagaaccaaggatggtcatgtacacaaagaccatcgagac | |
| | | ggccattcttgtttacaaaacacttaccaagaaagcactttgtaggggaactttagtaagttcttct | |
| | | catttcattatgtttcttccaaggaaacaggagagactgaattaataattctctctttcctcttaagc | |
| | | acttttaaaataataaagtacatcttgaaatttggggaggcatctctgatttaaaaaaagaaaaag | |
| | | gctgcttgatgtatgttatgcagagacactctgcctctggtggctgcagagcaatacccaagcc | |
| | | tcatttggaaggctcaacatttggaattgcactttaattgattaatcctcaattcatgtggccttacg | |
| | | ggatggtgggtctgggaccccaattcattcttatctgccaaagaattatctagaagcacatcaa | |
| | | ataccagcaccccacctgctcaatgggggtggaaaacttttgtatccctaagcatattattttata | |
| | | gtgtctgccatgccatgtggaaatactttatttttaacctcaggatttaaataaagtaaacactatg | |
| | | acatttagaca | |
| HNRNPU | NM_004501.3 | ctcgcgccaggcgagtctccgcgtctccctcgcgaactcggtgaaaggaattggcgccgttc | 12 |
| | | gacaccaggcggatccgctctgcagcacgaacccatctccagccgcagccgcagccgccg | |
| | | cccgggccgaggagcagccgcagcagccgccaccagtggccgagtgagcggagccgag | |
| | | tttgaggcagcgcctagcggtgaatcggggccctcaccatgagttcctcgcctgttaatgtaaa | |
| | | aaagctgaaggtgtcggagctgaaagaggagctcaagaagcgacgcctttctgacaagggt | |
| | | ctcaaggccgagctcatggagcgactccaggctgcgctggacgacgaggaggccggggg | |
| | | ccgccccgccatggagcccgggaacggcagcctagacctgggcggggattccgctgggc | |
| | | gctcgggagcaggcctcgagcaggaggccgcggccggcggcgatgaagaggaggagg | |
| | | aagaggaagaggaggaggaaggaatctccgctctggacggcgaccagatggagctagga | |
| | | gaggagaacggggccgcgggggcggccgactcgggcccgatggaggaggaggaggcc | |
| | | gcctcggaagacgagaacggcgacgatcagggtttccaggaaggggaagatgagctcgg | |
| | | ggacgaagaggaaggcgcgggcgacgagaacgggcacggggagcagcagcctcaacc | |
| | | gccggcgacgcagcagcaacagccccaacagcagcgcggggccgccaaggaggccgc | |
| | | ggggaagagcagcggccccacctcgctgttcgcggtgacggtggcgccgcccggggcga | |
| | | ggcagggccagcagcaggcgggaggggacggcaaaacagaacagaaaggcggagata | |
| | | aaaagaggggtgttaaaagaccacgagaagatcatggccgtggatattttgagtacattgaag | |
| | | agaacaagtatagcagagccaaatctcctcagccacctgttgaagaagaagatgaacacttc | |
| | | gatgacacagtggtttgtcttgatacttataattgtgatctacattttaaaatatcaagagatcgtct | |
| | | cagtgcttcttcccttacaatggagagttttgcttttctttgggctggaggaagagcatcctatggt | |
| | | gtgtcaaaaggcaaagtgtgttttgagatgaaggttacagagaagatcccagtaaggcatttat | |
| | | atacaaaagatattgacatacatgaagttcgtattggctggtcactaactacaagtggaatgtta | |
| | | cttggtgaagaagaattttcttatgggtattctctaaaaggaataaaaacatgcaactgtgagact | |
| | | gaagattatggagaaaagtttgatgaaaatgatgtgattacatgttttgctaactttgaaagtgat | |
| | | gaagtagaactctcgtatgctaagaatggacaagatcttggcgttgccttcaaaatcagtaagg | |
| | | aagttcttgctggacggccactgttcccgcatgttctctgccacaactgtgcagttgaatttaattt | |
| | | tggtcagaaggaaaagccatattttccaatacctgaagagtatactttcatccagaacgtcccct | |
| | | tagaggatcgagttagaggaccaaaggggcctgaagagaagaaagattgtgaagttgtgat | |
| | | gatgattggcttgccaggagctggaaaaactacctgggttactaaacatgcagcagaaaatcc | |
| | | agggaaatataacattcttggcacaaatactattatggataagatgatggtggcaggttttaaga | |
| | | agcaaatggcagatactggaaaactgaacacactgttgcagagagccccccagtgtcttggg | |
| | | aaatttattgagattgctgcccgaaagaagcgaaattttattctggatcagacaaatgtgtctgct | |
| | | gctgcccagaggagaaaaatgtgcctgtttgcaggcttccagcgaaaagctgttgtagtttgc | |
| | | ccaaaagatgaagactataagcaaagaacacagaagaaagcagaagtagaggggaaaga | |
| | | cctaccagaacatgcggtcctcaaaatgaaaggaaactttaccctcccagaggtagctgagtg | |
| | | ctttgatgaaataacctatgttgaacttcagaaggaagaagcccaaaaactcttggagcaatat | |
| | | aaggaagaaagcaaaaaggctcttccaccagaaaagaaacagaacactggctcaaagaaa | |
| | | agcaataaaaataagagtggcaagaaccagtttaacagaggtggtggccatagaggacgtg | |
| | | gaggattcaatatgcgtggtggaaatttcagaggaggagcccctgggaatcgtggcggatat | |
| | | aataggaggggcaacatgccacagagaggtggtggcggtggaggaagtggtggaatcgg | |
| | | ctatccataccctcgtgcccctgtttttcctggccgtggtagttactcaaacagagggaactaca | |
| | | acagaggtggaatgcccaacagagggaactacaaccagaacttcagaggacgaggaaaca | |
| | | atcgtggctacaaaaatcaatctcagggctacaaccagtggcagcagggtcaattctggggtc | |
| | | agaagccatggagtcagcattatcaccaaggatattattgaatacccaaataaaacgaactgat | |
| | | acatatttctccaaaaccttcacaagaagtcgactgttttctttagtaggctaactttttaaacattc | |
| | | cacaagaggaagtgcctgcgggttccttttttagaagctttgtgggttgattttttttcttttctttttt | |
| | | gtacatttttaattgcagtttaaaagtgaatcgtaagagaacctcagcattgtgcacgataagag | |
| | | aatgtgtcagtatttcagggttctacattttatctgtaaaatgtgacttttttttttttttatcacaacag | |
| | | aagtaaaatgttgctttgtacctggtgtcttttattaagaatttactccccccatttctcacagagaa | |
| | | taacagtcgggagtcattgtcacaatataatagaaatgttagcaaccagattcatgtaaggacta | |
| | | agtggtcctcatgaattgcattaagactctgtactgctcatattacactccatcctctctgtagtttg | |
| | | ctgggtagtggagggggtaagctaaatcatagtttctgacaataactgggaaggttttttcttaa | |
| | | aataacaatggaattggtataattgggattgaaaactaaaacttggaactaagatagagaagat | |
| | | ggagtgtatgtagaagggctgttaaaaatgtaaaacttggttgcattatttgtggaggctcaaac | |
| | | ttgtgaaggttaataccataatttttccatttgttctgcattttgattctgaaaagaaagctggctttg | |
| | | cccatttcttattaaaaaaacttgttgtaaatccagttgtctaatgggatctatatgaagttagccat | |
| | | gtctgtatgcccttctcccacaaaatactgtataactagtgtgcttgtagtagttaactccaccatc | |
| | | tttgtaagctaatgaaattgtgagtcacccatttatatcttaatttttaatcatgtcagttcttgaatgg | |
| | | gtatctccttagcctgctgatttctttttctttctaaagaaagtgggtggagaaattaatttagacgtt | |
| | | tgtttgcaataaaaagaattcattttactcttgttttgggattctcgccatcaaggttcaaaatccctt | |
| | | tatataactcccaagaggagaaatttattaagtgtgtgctttctggacagcttattctttactctgca | |
| | | tagaacatttaggttttaaaaacttaaatgtatactgacaattgatacataattatgaagtaaagttg | |
| | | aattcttcccttcccctcccccccagacaacttttaacatatttaatgaggggaaaaggtactgg | |
| | | ctgggagaagttaacactgagtttatcatctttacagaatgctaatgctgtcctcaactgattatttt | |
| | | atatacatatatatgatacatgaaactctgggatcagatgcttttagaagccatcatgcaagcca | |
| | | gtcattgatgtcactgctacacaacactgctaacttgactgtagctatgtaataacattagatccc | |
| | | ctaattgtaattatattgggtttgcacagaacactttaatcttcccctcaccaatgtgaagtgagga | |
| | | atcaggagtcaaactgtagaactaaaatttgacttcagtctagcgtttccttggtgtttttaggttg | |
| | | ctttggtaagtttaggtttgctatatttctgattgcttagaattttgttttagccctttaaaatcagatca | |
| | | taaatatgaattcatacttctaaggaattttcttgctataagctggagtttaggtgatgtataggttc | |
| | | agttgagacatttttggaacaggcaaatccttagttaacataagatatttaacagttgaagatagt | |
| | | gtcatggatttttatcttttttagcaagtaatgctaagaaccactggcctgagctactactcttcagt | |
| | | atacattattaggattgcatagacttactagaggaacagtttcaggttttgatgctaatcagtgttg | |
| | | tgtcctaaagttgtcctttgtgcctttaaaaagttttggatatatcttctagtttaaaattgcttattaa | |
| | | ggaattcattttataattgcagtgggaaagtaatggtcaagtaacactaggtagactatcatgcc | |
| | | tgtttagcccagagaatttggggggagagagaatagataaaaatggcacccagaaaaatgtt | |
| | | aaaatctttagtcaagactagaattaatacaattgtctacacttgtatggcagaaataaccttataa | |
| | | agtgtttaaggaattcagagaagggaatgtaccaaataagcaacagggagaaaattaggtaa | |
| | | gaagtaagatacgaacgagaaacctgatttattgctcatccttcccttgcctccctaatggcaag | |
| | | caaaactctgaacatctgaaaaggatgtagttctggacaaatcctgactacccagaggaaact | |
| | | cactgtgagattgctgttgatttgaagggtgctttcactaaggttatattttaaagtagaataacac | |
| | | atgctgagtgtaaactggctttggattggtcagctgcagtagtacaaaaacagcatagaatttg | |
| | | agaaaactaaaactgctatgagatagctatgagaaaactaaaactgctatgagatagaaatgat | |
| | | gtaaaattatgtggaaagttttccctcatatactcacatacagcctttgaagggctctggctctga | |
| | | ccggttgatggccttgagcgagatgaaatcatgaaattgagtcaaatcaatttgacattgaaatg | |
| | | acaagaggaaactcttaaatacataaaaacaagctctcatttgcctaggatagatactgtcttaa | |
| | | aaataaagactgaacctagatgttctgagcactagcaacaaggtattttaacaagtttaaagga | |
| | | attctctgaaaaagttataaaattattctaggaaacataaccataatagtgttttaagggactttca | |
| | | cctggggattttatattcatgaacagagtgtattctgtatttaaaatgtctcatttgtgggaattgga | |
| | | tgacatgttttttgataaatttattcacaatataaattgactttttattctaggaccatgtgaataatgg | |
| | | gttccattgcacaaatacaaatattttaatagcttcttaggcagtggtgtagacatcttggatataa | |
| | | ataattgtagatcttgtatatttgatttttaaaaaactagaataaacagagaggcataaacatatctt | |
| | | agagtccaagtggtagtgtttagcattggatataataaatggatgttttacaaagtgtttccataat | |
| | | tctcttcctatacataaatgtcttgttttcaaaagtggatggaacttggctgggtgtggtggctcac | |
| | | gcctgtaatcctagcactttgggaggccaaggcgggaggatcacttgagctcaggagtttga | |
| | | gaccatcctgggcaacatagtgagacctggtctcttgaaaaaaaaaaagtggatggaacttgt | |
| | | agcagagaattttatctacttctcaacctgcttcagaatacccatttgagatgttcccctggaaag | |
| | | atgaacacaatactgcatctgaagccatttcttcccacctaacattcttaaaatgattagagtctaa | |
| | | actttgtcattcattcctaattctggagctctgaggttgaggtgttcagagtttggtgaataattgg | |
| | | gtttaagttttaacattttagtaataataaaagcaaacacatacatgttaaggcctgacaataggtt | |
| | | gcaatacccatgcattgggactcatacccagcataaatggtgagggactgaacattagtgcttt | |
| | | gagcaagaattggttaactacctgagatctctaaaacagtaatttgaattactaatacataaacca | |
| | | caagtctcttcgaattgttaattgcctaaatttaccctaaaacttagttaacgctgcagtgcctttat | |
| | | taaagcttttttttgggggggggtgtgggcacagctactccataactttaaattttaaagcatgaa | |
| | | ggtgatctagctagtgttagtgcttgagttggtagttacataggctgctcttgaatcgtcctgttttc | |
| | | tttgcctagtattagcaccacaggtacatatttgtaaaataagcattaaaagtactttgtccagc | |
| HPN | NM_002151.2 | tcgagcccgctttccagggaccctacctgagggcccacaggtgaggcagcctggcctagca | 13 |
| | | ggccccacgccaccgcctctgcctccaggccgcccgctgctgcggggccaccatgctcctg | |
| | | cccaggcctggagactgacccgaccccggcactacctcgaggctccgcccccacctgctgg | |
| | | accccagggtcccaccctggcccaggaggtcagccagggaatcattaacaagaggcagtg | |
| | | acatggcgcagaaggagggtggccggactgtgccatgctgctccagacccaaggtggcag | |
| | | ctctcactgcggggaccctgctacttctgacagccatcggggcggcatcctgggccattgtgg | |
| | | ctgttctcctcaggagtgaccaggagccgctgtacccagtgcaggtcagctctgcggacgct | |
| | | cggctcatggtctttgacaagacggaagggacgtggcggctgctgtgctcctcgcgctccaa | |
| | | cgccagggtagccggactcagctgcgaggagatgggcttcctcagggcactgacccactcc | |
| | | gagctggacgtgcgaacggcgggcgccaatggcacgtcgggcttcttctgtgtggacgagg | |
| | | ggaggctgccccacacccagaggctgctggaggtcatctccgtgtgtgattgccccagagg | |
| | | ccgtttcttggccgccatctgccaagactgtggccgcaggaagctgcccgtggaccgcatcg | |
| | | tgggaggccgggacaccagcttgggccggtggccgtggcaagtcagccttcgctatgatgg | |
| | | agcacacctctgtgggggatccctgctctccggggactgggtgctgacagccgcccactgct | |
| | | tcccggagcggaaccgggtcctgtcccgatggcgagtgtttgccggtgccgtggcccaggc | |
| | | ctctccccacggtctgcagctgggggtgcaggctgtggtctaccacgggggctatcttcccttt | |
| | | cgggaccccaacagcgaggagaacagcaacgatattgccctggtccacctctccagtcccct | |
| | | gcccctcacagaatacatccagcctgtgtgcctcccagctgccggccaggccctggtggatg | |
| | | gcaagatctgtaccgtgacgggctggggcaacacgcagtactatggccaacaggccgggg | |
| | | tactccaggaggctcgagtccccataatcagcaatgatgtctgcaatggcgctgacttctatgg | |
| | | aaaccagatcaagcccaagatgttctgtgctggctaccccgagggtggcattgatgcctgcca | |
| | | gggcgacagcggtggtccctttgtgtgtgaggacagcatctctcggacgccacgttggcggc | |
| | | tgtgtggcattgtgagttggggcactggctgtgccctggcccagaagccaggcgtctacacc | |
| | | aaagtcagtgacttccgggagtggatcttccaggccataaagactcactccgaagccagcgg | |
| | | catggtgacccagctctgaccggtggcttctcgctgcgcagcctccagggcccgaggtgatc | |
| | | ccggtggtgggatccacgctgggcctaggatgggacgtttttcttcttgggcccggtccacag | |
| | | gtccaaggacaccctccctccagggtcctctcttccacagtggcgggcccactcagccccga | |
| | | gaccacccaacctcaccctcctgacccccatgtaaatattgttctgctgtctgggactcctgtct | |
| | | aggtgcccctgatgacgggatgctctttaaataataaagatggttttgattaaaaaaaaaaaaaa | |
| | | aaaaaaaaaaaa | |
| KRT15 | NM_002275.3 | cactcaaggtgtgcaggcagctgtgtttgtcaggaaggcagaaggagttggctttgctttagg | 14 |
| | | ggaggagacgaggtcccacaacaccctctgaagggtatataaggagccccagcgtgcagc | |
| | | ctggcctggtacctcctgccagcatctcttgggtttgctgagaactcacgggctccagctacct | |
| | | ggccatgaccaccacatttctgcaaacttcttcctccacctttgggggtggctcaacccgaggg | |
| | | ggttccctcctggctgggggaggtggctttggtggggggagtctctctgggggaggtggaa | |
| | | gccgaagtatctcagcttcttctgctaggtttgtctcttcagggtcaggaggaggatatgggggt | |
| | | ggcatgagggtctgtggctttggtggaggggctggtagtgttttcggtggaggctttggaggg | |
| | | ggcgttggtgggggttttggtggtggctttggtggtggcgatggtggtctcctctctggcaatg | |
| | | agaaaattaccatgcagaacctcaatgaccgcctggcctcctacctggacaaggtacgtgcc | |
| | | ctggaggaggccaatgctgacctggaggtgaagatccatgactggtaccagaagcagaccc | |
| | | caaccagcccagaatgcgactacagccaatacttcaagaccattgaagagctccgggacaa | |
| | | gatcatggccaccaccatcgacaactcccgggtcatcctggagatcgacaatgccaggctgg | |
| | | ctgcggacgacttcaggctcaagtatgagaatgagctggccctgcgccagggcgttgaggct | |
| | | gacatcaacggcttgcgccgagtcctggatgagctgaccctggccaggactgacctggaga | |
| | | tgcagatcgagggcctgaatgaggagctagcctacctgaagaagaaccacgaagaggaga | |
| | | tgaaggagttcagcagccagctggccggccaggtcaatgtggagatggacgcagcaccgg | |
| | | gtgtggacctgacccgtgtgctggcagagatgagggagcagtacgaggccatggcggaga | |
| | | agaaccgccgggatgtcgaggcctggttcttcagcaagactgaggagctgaacaaagaggt | |
| | | ggcctccaacacagaaatgatccagaccagcaagacggagatcacagacctgagacgcac | |
| | | gatgcaggagctggagatcgagctgcagtcccagctcagcatgaaagctgggctggagaa | |
| | | ctcactggccgagacagagtgccgctatgccacgcagctgcagcagatccaggggctcatt | |
| | | ggtggcctggaggcccagctgagtgagctccgatgcgagatggaggctcagaaccaggag | |
| | | tacaagatgctgcttgacataaagacacggctggagcaggagatcgctacttaccgcagcct | |
| | | gctcgagggccaggatgccaagatggctggcattggcatcagggaagcctcttcaggaggt | |
| | | ggtggtagcagcagcaatttccacatcaatgtagaagagtcagtggatggacaggtggtttctt | |
| | | cccacaagagagaaatctaagtgtctattgcaggagaaacgtcccttgccactccccactctc | |
| | | atcaggccaagtggaggactggccagagggcctgcacatgcaaactccagtccctgccttca | |
| | | gagagctgaaaagggtccctcggtcttttatttcagggctttgcatgcgctctattccccctctgc | |
| | | ctctccccaccttctttggagcaaggagatgcagctgtattgtgtaacaagctcatttgtacagt | |
| | | gtctgttcatgtaataaagaattacttttccttttgcaaataaaaaaaaaaaaaaaaaaaaaa | |
| KRT23 | NM_001282433.1 | aactagcacgtggctcccgcccccagactgcttctttattcattcctaacctttaccttggcagat | 15 |
| | | gaaatataagattcatcaaccacatttgacagcccatggcaggtttcctgttttccatcgtccctct | |
| | | gcaggtcacagacacacagagcccagccgtggcaggctcagccggggtccggggctgct | |
| | | aacaacggctacattcctcccccagggccaagggaaatcctgagcgcaggccagggttgttt | |
| | | ggttttgaggtgtgctgggatgaaaggcaccctggaagtggaaggttcggtcattcattaatta | |
| | | attacatctataattgagggtttgttcttaagagcgagtcctttgaaagtactttccttcaaacagtg | |
| | | actgccacaaaggcatcagatattcaccaccttctcggctgcctcagcacagcaagctttattct | |
| | | gggacctgagatcctgttctgagctggctttcccttctccaggctcgctcaccctccctttagag | |
| | | atagtggatggtaagatgaccaatgctcagattattcttctcattgacaatgccaggatggcagt | |
| | | ggatgacttcaacctcaagtatgaaaatgaacactcctttaagaaagacttggaaattgaagtc | |
| | | gagggcctccgaaggaccttagacaacctgaccattgtcacaacagacctagaacaggagg | |
| | | tggaaggaatgaggaaagagctcattctcatgaagaagcaccatgagcaggaaatggagaa | |
| | | gcatcatgtgccaagtgacttcaatgtcaatgtgaaggtggatacaggtcccagggaagatct | |
| | | gattaaggtcctggaggatatgagacaagaatatgagcttataataaagaagaagcatcgaga | |
| | | cttggacacttggtataaagaacagtctgcagccatgtcccaggaggcagccagtccagcca | |
| | | ctgtgcagagcagacaaggtgacatccacgaactgaagcgcacattccaggccctggagatt | |
| | | gacctgcagacacagtacagcacgaaatctgctttggaaaacatgttatccgagacccagtct | |
| | | cggtactcctgcaagctccaggacatgcaagagatcatctcccactatgaggaggaactgac | |
| | | gcagctacgccatgaactggagcggcagaacaatgaataccaagtgctgctgggcatcaaa | |
| | | acccacctggagaaggaaatcaccacgtaccgacggctcctggagggagagagtgaagg | |
| | | gacacgggaagaatcaaagtcgagcatgaaagtgtctgcaactccaaagatcaaggccata | |
| | | acccaggagaccatcaacggaagattagttctttgtcaagtgaatgaaatccaaaagcacgca | |
| | | tgagaccaatgaaagtttccgcctgttgtaaaatctattttcccccaaggaaagtccttgcacag | |
| | | acaccagtgagtgagttctaaaagatacccttggaattatcagactcagaaacttttatttttttttt | |
| | | ctgtaacagtctcaccagacttctcataatgctcttaatatattgcacttttctaatcaaagtgcga | |
| | | gtttatgagggtaaagctctactttcctactgcagccttcagattctcatcattttgcatctattttgt | |
| | | agccaataaaactccgcactagctgcaaaaaaaaaaaaaaaaaaa | |
| MAN2B2 | NM_001292038.1 | ggaagtgggcctggcaccttcccggcctgccgcagggatggggcagctgtgctggctgcc | 16 |
| | | gctgctggcaccgctcctgttgctgcgaccgccaggggtccagtccgccggccccatccgg | |
| | | gccttcgtggtgccccacagccacatggacgtgggctgggtctacactgtgcaggaaagcat | |
| | | gcgggcgtacgccgccaatgtctacacctcagtggtggaagagctggcccgcggccagca | |
| | | gcgccggttcatcgctgtggagcaggagtttttccggctgtggtgggatggcgtcgcctcgga | |
| | | ccagcagaaataccaggtccgccagctcctggaggaaggacgcctggaatttgtcatcgga | |
| | | ggccaggtcatgcatgacgaggctgtgacgcaccttgatgaccagatcctgcagctcacaga | |
| | | aggacacgggtttctctatgaaacatttgggatccggccacagttctcctggcacgttgacccg | |
| | | tttggcgcctctgccacgacgcccaccctatttgcgctggcgggcttcaatgcccacctcggc | |
| | | tcccggatcgactacgacctgaaggcagccatgcaggaggcccgggggctgcagttcgtgt | |
| | | ggcgagggtccccatccctctcagagcggcaggaaatcttcacgcacatcatggaccagtac | |
| | | agctactgcaccccgtcccacatccctttctccaacaggtcaggattttactggaatggcgtgg | |
| | | ctgtcttccccaagcctccccaagatggggtgtaccccaacatgagtgagcctgtcaccccag | |
| | | ccaacatcaacctctatgccgagctcggtgtctcggtgcagtatgccacgctgggcgactact | |
| | | tccgtgccctgcacgctctcaatgtcacctggcgtgtccgcgaccaccacgacttcctgcccta | |
| | | ttccacagaaccattccaggcctggacgggcttctacacgtcccgcagctcactgaaggggc | |
| | | tggcccggcgagccagcgccttgttgtatgccggggagtccatgttcacacgctacctgtgg | |
| | | ccggccccccgtgggcatctggaccccacctgggccctgcagcagctccagcagcttcgct | |
| | | gggccgtctccgaggtccagcaccatgatgccatcactgggactgagtcccccaaggtgag | |
| | | agacatgtacgcaacgcacctggcctcggggatgctgggcatgcgcaagctgatggcctcc | |
| | | atcgtcctagatgagctccagccccaggcacccatggcggccagctccgatgcaggacctg | |
| | | caggacattttgcctcggtctacaacccgctggcctggacggtcaccaccatcgtcaccctga | |
| | | ctgttggtttccctggagtccgcgtcacagatgaggcgggccacccagtgccctcgcagatc | |
| | | cagaactcaacagagaccccatctgcgtatgacctgcttattctgaccacaatcccaggcctca | |
| | | gttaccggcactacaacatcagacccactgcaggggcccaagagggcacccaggagccgg | |
| | | ctgccactgtggcgagcacccttcaatttggccgcaggctgaggagacgcaccagccatgc | |
| | | gggcaggtacttggtgcctgtggcaaacgactgctacattgtgctgctcgaccaggataccaa | |
| | | cctgatgcacagcatctgggagagacagagtaaccgaacggtgcgcgtgacccaggaattc | |
| | | ctggagtaccacgtcaacggggatgtgaaacagggccccatttccgataactacctgttcaca | |
| | | ccgggcaaggccgcggtgcctgcgtgggaagctgtggaaatggagattgtggcgggacag | |
| | | cttgtgactgagatccggcagtacttctacaggaacatgacagcacagaattacacgtatgca | |
| | | atccgctcccggctcacccatgtgccgcagggccatgacggggagctgctctgccaccgga | |
| | | tagagcaggagtaccaagccggccccctggagctgaaccgtgaggctgtcctgaggacca | |
| | | gcaccaacctaaacagccagcaggtcatctactcagacaacaacggctaccagatgcagcg | |
| | | gaggccctacgtttcctatgtgaacaacagcatcgcccggaattactaccccatggttcagtcg | |
| | | gccttcatggaggatggcaaaagcaggcttgtgttgctgtcggagcgggcacatggcatctc | |
| | | cagccaagggaatgggcaggtggaggtcatgctccaccggcggctgtggaacaacttcga | |
| | | ctgggacctgggctacaacctcacgctgaacgacacctcagtcgtccacccagtgctctggct | |
| | | tctgctgggatcctggtccctcaccactgccctgcgccagaggagcgcactggcgctgcagc | |
| | | acaggcccgtggtgctgttcggagacctcgctgggactgcgccgaagctcccaggacccca | |
| | | gcagcaagaggccgtgacgctgcccccgaatcttcacctgcagatcctgagcatccctggct | |
| | | ggcgctacagctccaaccacacggagcactctcagaatctccggaaaggccatcgagggg | |
| | | aagcccaggctgacctccgccgtgtcctgctgcggctctaccacctgtatgaagtgggcgag | |
| | | gacccagtcctgtctcagccagtgacagtgaatctggaggctgtgctgcaggcgctggggtc | |
| | | cgtggtggcagtggaggagcgctcgctcacagggacctgggatttgagcatgctgcaccgc | |
| | | tggagctggaggacggggcctggccgccacagaggtgacaccacctctccctcgaggcca | |
| | | ccaggaggccccatcatcaccgtccacccaaaggaaatccggacgttctttattcactttcaac | |
| | | agcagtgagccctgggcagatgccccggccccagggcttcccccaggaactccatgtaaca | |
| | | gaacagacccaggacagggaaaagcagtgcggagggatgggactggggagtcagctgct | |
| | | catctgcaggctaatggcaggaaatggtcatatttggggtttttccctaatttttttaaacaaaaatt | |
| | | acattacaagatccaggttcttcccccccacactcaatcaagccagccctctcctcttctgtcac | |
| | | gtaaaggatatttggcacactcatgcgtcattcattcacaaaacacaaacccaggactttctgcc | |
| | | taaggcagagcacaagactcacagcagcaccgaagcgcatctgccgtccgggccctgcca | |
| | | ggcttgccaggctgccagtggtaactgtggacctactgcgtgccacgtgttttcatagactcat | |
| | | cccatgctggcaacagccctgcaaggggcttggctctgccacagggcaggagaggaagtt | |
| | | gtagcgcctagcgagagttccagccccagacgcccacctgtgcctcagggcaccgcctgcc | |
| | | gagcagagaaggcacagcagccgtcagagtccatgagaggtgaaaccacacagcaggga | |
| | | tgtccaatatcagaactattaatatcaataaaagtataaccttcccaggtctatgcccaagagaat | |
| | | tgaaaacatccatccacacaatacctgtgctcccgcgttcatagcagcattactcaaaagtcaa | |
| | | acggtagcaacaacccaaatgtccatccacagatgaattaagacatgaagtgtgttctgtccat | |
| | | acaatggaatattatttggccataaaaaggaaggaaattctgacgcatgccacagcctgagtg | |
| | | aatcctacaaatattacgctaagtgaaagaagccaatcacgagtttatgtgaaatgtccagaata | |
| | | ggcaaatctgtgtatcagagacaaagcacattggtggttgccaggtactggaggaagagaga | |
| | | agaggcatgacagctaacagggacgggctttctttggaagatgatgaaattgtggaatgatgg | |
| | | ttgcacaactttgtgaatatactagaaaccaatgaattaaaaactttggaagatgaaaaaaaaaa | |
| | | aaaaaaaa | |
| MAX | NM_001320415.1 | tgcgtgccgccgttgatccggtgctgcagtgaggaggtggttcttgcccgtgttgtgtgtgtgt | 17 |
| | | gtgagtgagagagcgagtgagtgagtgagtgagtgtgtgtgtgggggggactcggcttgttg | |
| | | ttgtcggtgacttccccctccccttcaccccttcccctccccgccgccgctgcagtggccgctc | |
| | | cctgggccgtaggaaatgagcgataacgatgacatcgaggtggagagcgacgaagagcaa | |
| | | ccgaggtttcaatctgcggctgacaaacgggctcatcataatgcactggaacgaaaacgtag | |
| | | ggaccacatcaaagacagctttcacagtttgcgggactcagtcccatcactccaaggagaga | |
| | | aggcatcccgggcccaaatcctagacaaagccacagaatatatccagtatatgcgaaggaaa | |
| | | aaccacacacaccagcaagatattgacgacctcaagcggcagaatgctcttctggagcagca | |
| | | aggggaaagcgagagctgatcaagttctttgttcctggggaattcacttctcttcctccctcatg | |
| | | gaagatgcaagtaaaaggaaattccgtgcactggagaaggcgaggtcaagtgcccaactgc | |
| | | agaccaactacccctcctcagacaacagcctctacaccaacgccaagggcagcaccatctct | |
| | | gccttcgatgggggctcggactccagctcggagtctgagcctgaagagccccaaagcagga | |
| | | agaagctccggatggaggccagctaagccactcggggcaggccagcaataaaaactgtctg | |
| | | tctccatcgtctcatcctcctttcagttcgttggtagagccctcagaaccatttaagagactctttat | |
| | | ttttctctttctcccttttttttttaaatttttatttttacgtagaagctcttggacaacagctctcgttctc | |
| | | cttccccatttccactgtatattttttaatgtattcccttcagggattccctgtccccaacaggaattt | |
| | | ttaaaccaaaacaccccaacttggcagctttttctgtggaggacagacggccggccggacctc | |
| | | tgagcacatagtgtcctgcccaccctaccagctcctccagccctgccgggcacatgcccggg | |
| | | ggacgcctgccctgcccaggtggcctcctggcccgccctcacctctgatagactttgtgaatc | |
| | | tgaactgctctactttgagaagatgaccggtttggagtaatcagaatgaaccctcctcctttttaa | |
| | | gggttttttttttttcctttttctaaaaagctatgtatcgctcctattgaaagaccagatccttagaga | |
| | | agtttgtggtataaaaaggaagtggggacagattcgcagcacagagtcgctggcatgtttcac | |
| | | tcctgcttctctcagccagctgtttaagcctgcggcgccagcctcacggagggccgtgtgaca | |
| | | ctctcgtggtatgtatgggagatggcagcagtgaagcagcagccaccagggagtggccattt | |
| | | ggggttgggacagggagggtgttttgggtggcatagaggttttgtattgagggccagtgatga | |
| | | tgttttgatatttatttcctgctacttaaatttgaatctgagtgaattgtacctatttctgatgatgtcgg | |
| | | tcttgcaaagcgacagattcataaagtaatgatgaaatctttctttcttcccgtgtgtatttctaaga | |
| | | aatagagccaactgattttgtatgtaaataccaagagcaatttacctggtactaaacccgcaccc | |
| | | cagtgcggacccttcccagccctcatcccacttcctttcctactgtcctggaacctgtctccattg | |
| | | tgtgatccagccctggttctggctgtggtcagcagatgccagtgaagggttttgtgtgtttaggc | |
| | | ctcatttctttgtctttttcctactccgttcctggcatttgctgatttctagtgtatactctgtagtctca | |
| | | gttcgtgtttgattccattccatggaaataaaaagtatgttgtacatactgccgaagaattgtcttg | |
| | | caagttaaggcttccccctttactataagactataaataaaaacttattttatccttcttaaaaaaaa | |
| | | aaaaaaaa | |
| MRPS25 | NM_022497.4 | gaagcgtctcctgtgcgtctgcgcgggaagtggaacctggctctggggagaagccgcgtga | 18 |
| | | gatccgcgcgggtgctagctagtcctttctcgtcgctgctcggctcgcggcccgtggggtcg | |
| | | gccccgccaccgttgccgccatgcccatgaagggccgcttccccatccgccgcaccctgca | |
| | | atatctgagccaggggaacgtggtgttcaaggactccgtgaaggtcatgacagtgaattacaa | |
| | | cacgcatggggagctgggcgagggcgccaggaagtttgtgtttttcaacatacctcagattca | |
| | | atacaaaaacccttgggtgcagatcatgatgtttaagaacatgacgccgtcacccttcctgcga | |
| | | ttctacttagattctggggagcaggtcctggtggatgtggagaccaagagcaataaggagatc | |
| | | atggagcacatcagaaaaatcttggggaagaatgaggaaaccctcagggaagaggaggag | |
| | | gagaaaaagcagctttctcacccagccaacttcggccctcgaaagtactgcctgcgggagtg | |
| | | catctgtgaagtggaagggcaggtgccctgccccagcctggtgccattacccaaggagatg | |
| | | agggggaagtacaaagccgctctgaaagccgatgcccaggactaaggcccacggtcactgt | |
| | | gggctggggtgatggtgtctgaccagtggggagattggaatgggattactttggcccaggga | |
| | | agcccctggttctgtccctggagactctggaaatccttttgcattaaaaggactttacacacctgt | |
| | | gtaaaaggatgtgggagaggagggtctgaagctgagctgctaaatgaatatccctgctctgct | |
| | | ggtcaataaaacgcttcctaatagcagcttggcgtgtatctggtcctagtgaagaggaaggcct | |
| | | gtgtagcagaaaggctttgggcctgagaggttaaggccacagcctgttgacacctgttttggtc | |
| | | ctgcgaccctttactggtctccgctggctttgaatcttcctctgggctctactctggagaacataa | |
| | | gggctgctgtggttgagtctggctagcactgtctgtggttggcagtgtgtacacccctccgttca | |
| | | gttccttgggggtatttttcagaaatccaaaggcaacccttcgtgcagtgctcacttttttaagtac | |
| | | agttgattacccttgcctgctggggggcctagccatgggccagagatggaggagccccagt | |
| | | ggctgacaggccagcctcactcaggcacgtacctgctgaccagtcagccactgccaacccat | |
| | | ggcccagccactgtgtgcattagcagggaggtttgtaggccatggaggaaatgaggagaca | |
| | | ccacctagtggagacattggggccctgctggggggatggtgtctatagctggctctgctggct | |
| | | ccctcaggccctgcttaccaagctctggaggagggagtgctgcattactgagcaccttccttgt | |
| | | tctttcctcataggacactgatgttactgtcactttagttatgctaaagtggaggtttcagcctcca | |
| | | gaagacagcagagccttctagggtcaccttaagaataggtttagctaggctgggtgtggtggc | |
| | | tcatgcctgtaatcccagcactttgggaggctgatgtgggcggatagttgagcccaggagttc | |
| | | gagaccagcctgggcaacatggcaagaccttgtctctacaaaaagtacaaaaattagatgggt | |
| | | gtggtgtttcgcgtctgtagtcccagctatttgggaagctgaggtgggaggatctcgagcctgc | |
| | | gagatcaaggctgcagtgaatcatgatcgtgcgctactgcactccagcctgggccacagagc | |
| | | aagactgtctcaagaaaaaaaattttttaaataagtttaattataaagtgaaggaccagttggacc | |
| | | acggacccctagaaactcagccaaggagacctgactttatctgagacaggaaggcagtggtt | |
| | | aaaagaattaaaaaaggacagtggtgcttgacagagcaggactgaagttttcattcctctacct | |
| | | gctggggccttggtcaagtcctaaaagctttccaggtctgtaactggttttgtctgtaaaatggg | |
| | | gaaaaggttaccttttctgccacatgaagtagctggctggtgaaatgatgtcatactacgatgtg | |
| | | tttcattaactgttggagattatcacattaaattgagcagatttggatcttttacatttgatgctcaag | |
| | | tttgattctgtaccagatctgggtttggggtgatggcttgtcgggggtgggaggggaaggtgg | |
| | | gcagagactgatgtataatggacaggtgttccttgagtgacagacttacaaagggagtgtttaa | |
| | | ttgaaatctagcatcttgtctctctgctatcagcattttggctgggtggaaaaagctctcctccac | |
| | | agagagccctcccatgtctgctggtttcagttccccacccaactgactgaagtcttgaccttgat | |
| | | accacctgcctgggcgttctacttggctgcaagggctcttcagaaagcacctagttccttttggc | |
| | | tttcatctgcagtgtaggtctccatagctagtgacacacaaagccttatgagagtattccttccct | |
| | | ctagggcccaaagaggctgctaaccagcaggaatgtcctgaaccaaaaggagtggtgacca | |
| | | ttctctagacagaataaatgtctggtccctaatgggacagataactagggtttgagttcaggctt | |
| | | gacacaggagcaatgggcagtgaggtggacagcccagaccctgaaaggacgtgtggaga | |
| | | cagcagcagtcctgtggtgcagttactgcctgtgtggccagcttcacaggcagcagataattt | |
| | | gtggaaataaacattattttctttttttgttccctgtaggctacttgatcctggatagtttctctttaaa | |
| | | cctttgtctttccctattttgtttagtagtgatgaaagatgggtttaaaatatacagaaatgggaga | |
| | | aaattaattagagaaaatgtgaggattttataattgataacatgttcttaattttacttgcacaagca | |
| | | gtgttcacagccttattctgaagtgaccacttttcttctctttgaggcactacctggcagcaccatc | |
| | | taaacctgtatcatctgctgtggtcccagttactcaggaggctgaggcaggaggatcacttga | |
| | | gcccaggaggttgaggctggagtgagccatgactgtaccactgcattccagcctgggtgacg | |
| | | gagtgaggccctatctgcctgccagtgagttctgtcatgcatcagactttggacctcagaatttt | |
| | | cacctggaacatgagaggtcagatgacataatgaatatgttctaacttctgaaacttttggagctt | |
| | | gtttttgcccttgatagcacgtatccttagagaataagcttgccaatttaaggcctgtctgtaatgg | |
| | | catctgaatgttccagcagcctaactgtgctctgctccagaaacatgtgtgtggcttccctacct | |
| | | ccacaccaaggagctgccttgctgggtggatgtaaagactgtcctgccagggtttcccactaa | |
| | | aggtattaataagtccagtggaccacagagggcccaatccctcttggccgctaaggctaaggt | |
| | | aggagccagagagctgtagagacaggaccaccaggatgcttcatgtccataacacaagaga | |
| | | gcgctgcattgtttgggggccaggcattactactatcatgaagaaaaatgaagagcaggatgc | |
| | | atgttagtctttaattcacttttaatagtataaacctcatttaggtagtagtattaagccacaacaata | |
| | | atgccacattgaaacagcatttaataaaatgcataaagctaattcatgcactgcaatactctatat | |
| | | acaaacacaacaatgcaaattcttcttcaagactgaagacattgattagatataaaattcagttta | |
| | | aaaagaacatgctattttttaaatgccatcacataaacaaagtgatttcacagggagaagaaag | |
| | | ctgtataaagctgcagctttcaacaggttttaaacctggcattaaaatgtaatggcaaaaccaat | |
| | | attttctatattgtgaagggcaaaggttacagaaacggtcccaagaaaatctaacacccaaattt | |
| | | tcctttgatagatagatgcctttaaaagggctggtaatgcagttacattctaacagagaagtcca | |
| | | aactacaggtaaaaactacggcttgtactgtgaaaaatgtgcagcttttcagttataaaactagtt | |
| | | gaacactggtttacaagataatccgtagaacagagagactgtagaaaaatattccagcacttga | |
| | | gttgtgtgtggcagcagcatttgagtccatgaatgctatggtcattaaaataattgattatactttc | |
| | | ctaaagaaaagccattttttaatgcaaagtctactacttccaaatgttattccaaacattaaatttta | |
| | | acagtctatcaatcaataaatgttgaacattttttttctaaaaaaaaa | |
| NDUFS2 | NM_001166159.1 | aaaagccagccaggagctgtgggaggaaacgccctcagtaaagatgaccgcggtcactgtt | 19 |
| | | atctaaacgcaagtgaagccgagtcacaggacccggatgttgtcagttcgacggtaaacgac | |
| | | cctgccagcttccaagagggcggcttcactgtgcgaataggtgagaagccaagaaggaggc | |
| | | gcgctggagttacttccgcccggttctccttcccgcagtctgcagccggagtaagatggcggc | |
| | | gctgagggctttgtgcggcttccggggcgtcgcggcccaggtgctgcggcctggggctgga | |
| | | gtccgattgccgattcagcccagcagaggtgttcggcagtggcagccagatgtggaatggg | |
| | | cacagcagtttgggggagctgttatgtacccaagcaaagaaacagcccactggaagcctcca | |
| | | ccttggaatgatgtggaccctccaaaggacacaattgtgaagaacattaccctgaactttgggc | |
| | | cccaacacccagcagcgcatggtgtcctgcgactagtgatggaattgagtggggagatggtg | |
| | | cggaagtgtgatcctcacatcgggctcctgcaccgaggcactgagaagctcattgaatacaa | |
| | | gacctatcttcaggcccttccatactttgaccggctagactatgtgtccatgatgtgtaacgaac | |
| | | aggcctattctctagctgtggagaagttgctaaacatccggcctcctcctcgggcacagtggat | |
| | | ccgagtgctgtttggagaaatcacacgtttgttgaaccacatcatggctgtgaccacacatgcc | |
| | | ctggaccttggggccatgacccctttcttctggctgtttgaagaaagggagaagatgtttgagtt | |
| | | ctacgagcgagtgtctggagcccgaatgcatgctgcttatatccggccaggaggagtgcacc | |
| | | aggacctaccccttgggcttatggatgacatttatcagttttctaagaacttctctcttcggcttgat | |
| | | gagttggaggagttgctgaccaacaataggatctggcgaaatcggacaattgacattggggtt | |
| | | gtaacagcagaagaagcacttaactatggttttagtggagtgatgcttcggggctcaggcatc | |
| | | cagtgggacctgcggaagacccagccctatgatgtttacgaccaggttgagtttgatgttcctg | |
| | | ttggttctcgaggggactgctatgataggtacctgtgccgggtggaggagatgcgccagtcc | |
| | | ctgagaattatcgcacagtgtctaaacaagatgcctcctggggagatcaaggttgatgatgcc | |
| | | aaagtgtctccacctaagcgagcagagatgaagacttccatggagtcactgattcatcacttta | |
| | | agttgtatactgagggctaccaagttcctccaggagccacatatactgccattgaggctcccaa | |
| | | gggagagtttggggtgtacctggtgtctgatggcagcagccgcccttatcgatgcaagatcaa | |
| | | ggctcctggttttgcccatctggctggtttggacaagatgtctaagggacacatgttggcagat | |
| | | gtcgttgccatcataggtacgaggcctattgtgtagtagaggtatcctagacaaaggagttcgg | |
| | | gacgcccactggggacagaaggagaacacttcctgttcaccataggccatggcatggactc | |
| | | gggtcctcaatcttttgagcacagtaatgggttctggatcttgggtaacaccactttttttgtttgttt | |
| | | tgcctcacaacaggaagataagtaacatcacttttttcctccatcctctcacctaggtacccaag | |
| | | atattgtatttggagaagtagatcggtgagcaggggagcagcgtttgatcccccctgcctatca | |
| | | gcttcttctgtggagcctgttcctcactggaaattggcctctgtgtgtgtgtgtgtgtgtgtgtgtg | |
| | | tgtgtatgttcatgtacacttggctgtcaggctttctgtgcatgtactaaaaaaggagaaattataa | |
| | | taaattagccgtcttgcggcccctaggcctaaaaaaaaaaaaaaaaaa | |
| PPARGC1A | NM_013261.3 | tagtaagacaggtgccttcagttcactctcagtaaggggctggttgcctgcatgagtgtgtgct | 20 |
| | | ctgtgtcactgtggattggagttgaaaaagcttgactggcgtcattcaggagctggatggcgtg | |
| | | ggacatgtgcaaccaggactctgagtctgtatggagtgacatcgagtgtgctgctctggttggt | |
| | | gaagaccagcctctttgcccagatcttcctgaacttgatctttctgaactagatgtgaacgacttg | |
| | | gatacagacagctttctgggtggactcaagtggtgcagtgaccaatcagaaataatatccaatc | |
| | | agtacaacaatgagccttcaaacatatttgagaagatagatgaagagaatgaggcaaacttgc | |
| | | tagcagtcctcacagagacactagacagtctccctgtggatgaagacggattgccctcatttga | |
| | | tgcgctgacagatggagacgtgaccactgacaatgaggctagtccttcctccatgcctgacg | |
| | | gcacccctccaccccaggaggcagaagagccgtctctacttaagaagctcttactggcacca | |
| | | gccaacactcagctaagttataatgaatgcagtggtctcagtacccagaaccatgcaaatcac | |
| | | aatcacaggatcagaacaaaccctgcaattgttaagactgagaattcatggagcaataaagcg | |
| | | aagagtatttgtcaacagcaaaagccacaaagacgtccctgctcggagcttctcaaatatctga | |
| | | ccacaaacgatgaccctcctcacaccaaacccacagagaacagaaacagcagcagagaca | |
| | | aatgcacctccaaaaagaagtcccacacacagtcgcagtcacaacacttacaagccaaacca | |
| | | acaactttatctcttcctctgaccccagagtcaccaaatgaccccaagggttccccatttgagaa | |
| | | caagactattgaacgcaccttaagtgtggaactctctggaactgcaggcctaactccacccac | |
| | | cactcctcctcataaagccaaccaagataacccttttagggcttctccaaagctgaagtcctctt | |
| | | gcaagactgtggtgccaccaccatcaaagaagcccaggtacagtgagtcttctggtacacaa | |
| | | ggcaataactccaccaagaaagggccggagcaatccgagttgtatgcacaactcagcaagt | |
| | | cctcagtcctcactggtggacacgaggaaaggaagaccaagcggcccagtctgcggctgttt | |
| | | ggtgaccatgactattgccagtcaattaattccaaaacagaaatactcattaatatatcacagga | |
| | | gctccaagactctagacaactagaaaataaagatgtctcctctgattggcaggggcagatttgt | |
| | | tcttccacagattcagaccagtgctacctgagagagactttggaggcaagcaagcaggtctct | |
| | | ccttgcagcacaagaaaacagctccaagaccaggaaatccgagccgagctgaacaagcact | |
| | | tcggtcatcccagtcaagctgtttttgacgacgaagcagacaagaccggtgaactgagggac | |
| | | agtgatttcagtaatgaacaattctccaaactacctatgtttataaattcaggactagccatggat | |
| | | ggcctgtttgatgacagcgaagatgaaagtgataaactgagctacccttgggatggcacgca | |
| | | atcctattcattgttcaatgtgtctccttcttgttcttcttttaactctccatgtagagattctgtgtcac | |
| | | cacccaaatccttattttctcaaagaccccaaaggatgcgctctcgttcaaggtccttttctcgac | |
| | | acaggtcgtgttcccgatcaccatattccaggtcaagatcaaggtctccaggcagtagatcctc | |
| | | ttcaagatcctgctattactatgagtcaagccactacagacaccgcacgcaccgaaattctccc | |
| | | ttgtatgtgagatcacgttcaagatcgccctacagccgtcggcccaggtatgacagctacgag | |
| | | gaatatcagcacgagaggctgaagagggaagaatatcgcagagagtatgagaagcgagag | |
| | | tctgagagggccaagcaaagggagaggcagaggcagaaggcaattgaagagcgccgtgt | |
| | | gatttatgtcggtaaaatcagacctgacacaacacggacagaactgagggaccgttttgaagt | |
| | | ttttggtgaaattgaggagtgcacagtaaatctgcgggatgatggagacagctatggtttcatta | |
| | | cctaccgttatacctgtgatgcttttgctgctcttgaaaatggatacactttgcgcaggtcaaacg | |
| | | aaactgactttgagctgtacttttgtggacgcaagcaatttttcaagtctaactatgcagacctag | |
| | | attcaaactcagatgactttgaccctgcttccaccaagagcaagtatgactctctggattttgata | |
| | | gtttactgaaagaagctcagagaagcttgcgcaggtaacatgttccctagctgaggatgacag | |
| | | agggatggcgaatacctcatgggacagcgcgtccttccctaaagactattgcaagtcatactta | |
| | | ggaatttctcctactttacactctctgtacaaaaacaaaacaaaacaacaacaatacaacaaga | |
| | | acaacaacaacaataacaacaatggtttacatgaacacagctgctgaagaggcaagagaca | |
| | | gaatgatatccagtaagcacatgtttattcatgggtgtcagctttgcttttcctggagtctcttggt | |
| | | gatggagtgtgcgtgtgtgcatgtatgtgtgtgtgtatgtatgtgtgtggtgtgtgtgcttggttta | |
| | | ggggaagtatgtgtgggtacatgtgaggactgggggcacctgaccagaatgcgcaagggc | |
| | | aaaccatttcaaatggcagcagttccatgaagacacgcttaaaacctagaacttcaaaatgttc | |
| | | gtattctattcaaaaggaaatatatatatatatatatatatatatatatatatatataaattaaaaagg | |
| | | aaagaaaactaacaaccaaccaaccaaccaaccaaccacaaaccaccctaaaatgacagcc | |
| | | gctgatgtctgggcatcagcctttgtactctgtttttttaagaaagtgcagaatcaacttgaagca | |
| | | agctttctctcataacgtaatgattatatgacaatcctgaagaaaccacaggttccatagaactaa | |
| | | tatcctgtctctctctctctctctctctctctcttttttttttctttttccttttgccatggaatctgggtgg | |
| | | gagaggatactgcgggcaccagaatgctaaagtttcctaacattttgaagtttctgtagttcatc | |
| | | cttaatcctgacacccatgtaaatgtccaaaatgttgatcttccactgcaaatttcaaaagccttgt | |
| | | caatggtcaagcgtgcagcttgttcagcggttctttctgaggagcggacaccgggttacattac | |
| | | taatgagagttgggtagaactctctgagatgtgttcagatagtgtaattgctacattctctgatgta | |
| | | gttaagtatttacagatgttaaatggagtatttttattttatgtatatactatacaacaatgttcttttttg | |
| | | ttacagctatgcactgtaaatgcagccttcttttcaaaactgctaaatttttcttaatcaagaatattc | |
| | | aaatgtaattatgaggtgaaacaattattgtacactaacatatttagaagctgaacttactgcttat | |
| | | atatatttgattgtaaaaacaaaaagacagtgtgtgtgtctgttgagtgcaacaagagcaaaatg | |
| | | atgctttccgcacatccatcccttaggtgagcttcaatctaagcatcttgtcaagaaatatcctagt | |
| | | cccctaaaggtattaaccacttctgcgatatttttccacattttcttgtcgcttgtttttctttgaagttt | |
| | | tatacactggatttgttaggggaatgaaattttctcatctaaaatttttctagaagatatcatgatttt | |
| | | atgtaaagtctctcaatgggtaaccattaagaaatgtttttattttctctatcaacagtagttttgaaa | |
| | | ctagaagtcaaaaatctttttaaaatgctgttttgttttaatttttgtgattttaatttgatacaaaatgc | |
| | | tgaggtaataattatagtatgatttttacaataattaatgtgtgtctgaagactatctttgaagccag | |
| | | tatttctttcccttggcagagtatgacgatggtatttatctgtattttttacagttatgcatcctgtata | |
| | | aatactgatatttcattcctttgtttactaaagagacatatttatcagttgcagatagcctatttattat | |
| | | aaattatgagatgatgaaaataataaagccagtggaaattttctacctaggatgcatgacaattg | |
| | | tcaggttggagtgtaagtgcttcatttgggaaattcagcttttgcagaagcagtgtttctacttgca | |
| | | ctagcatggcctctgacgtgaccatggtgttgttcttgatgacattgcttctgctaaatttaataaa | |
| | | aacttcagaaaaacctccattttgatcatcaggatttcatctgagtgtggagtccctggaatgga | |
| | | attcagtaacatttggagtgtgtattcaagtttctaaattgagattcgattactgtttggctgacatg | |
| | | acttttctggaagacatgatacacctactactcaattgttcttttcctttctctcgcccaacacgatc | |
| | | ttgtaagatggatttcacccccaggccaatgcagctaattttgatagctgcattcatttatcacca | |
| | | gcatattgtgttctgagtgaatccactgtttgtcctgtcggatgcttgcttgattttttggcttcttattt | |
| | | ctaagtagatagaaagcaataaaaatactatgaaatgaaagaacttgttcacaggttctgcgtta | |
| | | caacagtaacacatctttaatccgcctaattcttgttgttctgtaggttaaatgcaggtattttaact | |
| | | gtgtgaacgccaaactaaagtttacagtctttctttctgaattttgagtatcttctgttgtagaataat | |
| | | aataaaaagactattaagagcaataaattatttttaagaaatcgagatttagtaaatcctattatgt | |
| | | gttcaaggaccacatgtgttctctattttgcctttaaatttttgtgaaccaattttaaatacattctcct | |
| | | ttttgccctggattgttgacatgagtggaatacttggtttcttttcttacttatcaaaagacagcact | |
| | | acagatatcatattgaggattaatttatcccccctacccccagcctgacaaatattgttaccatga | |
| | | agatagttttcctcaatggacttcaaattgcatctagaattagtggagcttttgtatcttctgcagac | |
| | | actgtgggtagcccatcaaaatgtaagctgtgctcctctcatttttatttttatttttttgggagagaa | |
| | | tatttcaaatgaacacgtgcaccccatcatcactggaggcaaatttcagcatagatctgtaggat | |
| | | ttttagaagaccgtgggccattgccttcatgccgtggtaagtaccacatctacaattttggtaacc | |
| | | gaactggtgctttagtaatgtggatttttttcttttttaaaagagatgtagcagaataattcttccagt | |
| | | gcaacaaaatcaattttttgctaaacgactccgagaacaacagttgggctgtcaacattcaaag | |
| | | cagcagagagggaactttgcactattggggtatgatgtttgggtcagttgataaaaggaaacct | |
| | | tttcatgcctttagatgtgagcttccagtaggtaatgattatgtgtcctttcttgatggctgtaatga | |
| | | gaacttcaatcactgtagtctaagacctgatctatagatgacctagaatagccatgtactataatg | |
| | | tgatgattctaaatttgtacctatgtgacagacattttcaataatgtgaactgctgatttgatggag | |
| | | ctactttaagatttgtaggtgaaagtgtaatactgttggttgaactatgctgaagagggaaagtg | |
| | | agcgattagttgagcccttgccgggccttttttccacctgccaattctacatgtattgttgtggtttt | |
| | | attcattgtatgaaaattcctgtgattttttttaaatgtgcagtacacatcagcctcactgagctaat | |
| | | aaagggaaacgaatgtttcaaatcta | |
| PPRC1 | NM_001288727.1 | gtagttcctttcccacaatcggctgggcgaggcggcgccagcgatcagagcagcgctgggt | 21 |
| | | gttcaggggccaagatggcggcgcgccggggacggagagacggagtcgcgccgccccc | |
| | | gagtgggggccccggtccggaccctggcgggggagcccgcggcagtggttggggaagtc | |
| | | gaagccaagcgccgtatgggactttgggcgctgtgagcggcggcgagcaggtgctgctgc | |
| | | atgaggaggcgggtgattctggctttgtcagtctctctcggctgggcccatctctgagggaca | |
| | | aggacctggaaatggaggagctaatgctgcaggatgagacactgctggggaccatgcaga | |
| | | gctacatggatgcctcccttatctccctcattgaggattttgggagccttggagagagcaggtta | |
| | | tctctggaggaccagaatgaagtgtcgctgctcacggctctgacggagatcttggacaatgca | |
| | | gattctgagaacctttctccatttgacagcattcctgattcggagctgcttgtgtcaccccggga | |
| | | gggctcctctctgcacaagctgcttactctctctcggacacccccagaacgtgacctcatcacc | |
| | | ccagttgacccactggggcccagtacaggcagcagtagagggagtggggttgaaatgtctct | |
| | | tccagatccctcttgggacttctccccaccctctttcttagagacctcttcccccaagcttcctag | |
| | | ctggagacccccaagatcaagaccacgctggggccaatccccacctccccagcagcgcag | |
| | | tgatggagaagaagaggaggaggtggccagcttcagtggccagattcttgccggggagctt | |
| | | gacaactgtgtgagcagtatcccggacttccccatgcatttggcctgccctgaggaggaagat | |
| | | aaagcaacagcagcagagatggcagtgccagcagctggtgatgagagcatctcctccctga | |
| | | gtgagctggtgcgggccatgcacccatactgcctgcccaacctcacccacctggcatcacttg | |
| | | aggatgagcttcaggagcagccagatgatttgacactgcctgagggctgcgtagtgctggag | |
| | | attgtggggcaggcagccacagctggcgatgacctggagatcccagttgtggtgcgacagg | |
| | | tctctcctggaccccggcctgtgctcctggatgactcgctagagactagttctgccttgcagct | |
| | | gcttatgcctacactggagtcagagacagaggctgctgtgcccaaggtaaccctctgctctga | |
| | | gaaagaggggttgtcattgaactcagaggagaagctggactcagcctgcttattgaagccca | |
| | | gggaggtcgtggagccggtggtgcccaaggagcctcagaacccacctgccaatgcagcac | |
| | | caggttcccagagagctcgaaagggcaggaagaagaagagcaaggagcagccagcagc | |
| | | ctgtgtggaaggctatgccaggaggctgaggtcatcttctcgcgggcagtctactgtaggtac | |
| | | agaagtgacctctcaggtagacaacttgcagaaacagcctcaggaagaacttcaaaaagagt | |
| | | ctgggcctctccagggtaaggggaagccccgggcttgggctcgggcctgggcagctgcctt | |
| | | ggagaattctagccctaagaacttggagagaagtgctggacaaagtagtcctgctaaagaag | |
| | | gccctctagacctctacccaaagctggctgacactatccaaaccaatcctataccaacccatct | |
| | | ctcattggtcgactctgcccaagccagccccatgccagttgactctgttgaagctgatcccact | |
| | | gcagttggccctgttctagctggccctgtacctgttgaccctgggttggttgaccttgcttcaac | |
| | | cagctcagaactggttgagcctctcccggctgagccagtgctgatcaacccagtcctggctga | |
| | | ctcagcagcagttgaccctgcagtggttcccatctcagataacttgccaccagttgatgctgtc | |
| | | ccgtctggcccagcaccagttgatctagcactggttgaccctgttcctaatgacctgactccag | |
| | | ttgacccagtgctagttaagtccagaccaactgatcccagacgtggtgcagtgtcatcagccct | |
| | | ggggggttcagcaccccagctcctcgtggagtcagagtccttggacccaccaaagaccatca | |
| | | tccctgaagtcaaagaggttgtggattctctgaaaattgaaagtggtaccagtgctacaaccca | |
| | | tgaagccagacctcggcctctcagcttatctgagtaccggcgacgaaggcagcaacgccaa | |
| | | gcagaaacagaagagagaagtccacagcccccaactgggaagtggcctagccttccagag | |
| | | actcccacagggctggcagacatcccttgtcttgtcatcccaccagccccagccaagaagac | |
| | | agctctgcagagaagccctgaaacaccccttgagatttgccttgtgcctgtaggtcccagccct | |
| | | gcttctcctagtcctgagccacctgtaagcaaacctgtggcctcatctcccactgagcaggtgc | |
| | | catcccaggagatgccactgttggcgagaccttcccctcctgtgcagtctgtgtcccctgctgt | |
| | | gcccacacctccctcgatgtctgctgccctgcctttccctgcaggtgggcttggcatgcccccc | |
| | | agtctgcccccacctcccttgcagcctcctagtcttccattgtctatggggccagtactacctgat | |
| | | ccgtttactcactatgcccccttgccatcctggccttgttatcctcatgtgtccccttctggctatcc | |
| | | ttgcctgccccccccaccaacggtgcccctagtgtctggtactcctggtgcctatgccgtgcct | |
| | | cccacttgcagtgtgccttgggcaccccctcctgccccagtctcaccttacagttccacatgtac | |
| | | ctatgggcccttgggatggggcccagggcctcaacatgctccattctggtctactgttccccca | |
| | | cctcctttgcctccagcctccattgggagagctgttccccaacctaaaatggagtctaggggca | |
| | | ctccagctggccctcctgaaaatgtacttcccttgtcgatggctcctcccctcagtcttgggcta | |
| | | cctggccatggagctcctcagacagagcctaccaaggtggaggtcaagccagtgcctgcat | |
| | | ctccccatccgaaacacaaggtgtctgccctggtgcaaagtccccagatgaaggctctagcat | |
| | | gtgtgtctgctgaaggtgtgactgttgaggagcctgcatcagagaggctaaagcctgagacc | |
| | | caagagaccaggcccagggagaagccccccttgcctgctaccaaggctgttcccacaccaa | |
| | | ggcagagcactgtccccaagctgcctgctgtccacccagcccgtctaaggaagctgtccttcc | |
| | | tgcctaccccacgtactcagggttctgaagatgtggtacaggctttcatcagtgagattggaatt | |
| | | gaggcatcggacctgtccagtctgctggagcagtttgagaaatcagaagccaaaaaggagt | |
| | | gtcctcctccggctcctgctgacagcttggctgtaggaaactcagggtccagctgtagttcctc | |
| | | tggacgttctcgaagatgctcttcctcttcttcgtcatcatcttcctcttcgtcttcctcatcctcatc | |
| | | atccagttctcgaagccgctcacgatccccatccccccgccggagaagtgacaggaggcgg | |
| | | cggtacagctcttatcgttcacatgaccattaccaaaggcaaagagtgctacaaaaggagcgt | |
| | | gcaatagaagaaagaagggtggtcttcattggaaagatacctggccgcatgactcgatcaga | |
| | | gctgaaacagaggttctccgtttttggagagattgaggagtgcaccatccacttccgtgtccaa | |
| | | ggggacaactacggcttcgtcacttatcgctatgctgaggaggcatttgcagccattgagagt | |
| | | ggccacaagctgcggcaggcagatgagcagccctttgatctctgctttgggggccgaaggc | |
| | | agttctgcaagaggagctattctgatcttgactccaaccgggaagactttgacccagcacctgt | |
| | | aaagagcaaatttgattctcttgactttgacacattgttgaaacaggcccagaagaacctcagg | |
| | | aggtaaccttgggcccttccctgctatcctttttctcctttggaggtgcccaacctcctccacccc | |
| | | cttcccctactctaggggagagagctgctagtgagatgactgttttataaagaaatggaaaaaa | |
| | | gtgaaataaaaaatatgttgaatcagattttttaaaaggggtatttgtttttttataacaggtattgaa | |
| | | acaagttaacttgcattcctatgtaagataggaggggctgaggggatccccagtgtttggaac | |
| | | ataagtcactatgcagactaataaacatcaactagagagaactcccaaaaaaaaaaaaaaaaaa | |
| RAD23A | NM_001270362.1 | ggtggtcagcgcgcgcattgcctgccccggaagtggtcggcgcgcggcgcggcgcgcct | 22 |
| | | gggcgctaagatggcggcggcgtgagttgcatgttgtgtgaggatcccggggccgccgcgt | |
| | | cgctcgggccccgccatggccgtcaccatcacgctcaaaacgctgcagcagcagaccttca | |
| | | agatccgcatggagcctgacgagacggtgaaggtgctaaaggagaagatagaagctgaga | |
| | | agggtcgtgatgccttccccgtggctggacagaaactcatctatgccggcaagatcttgagtg | |
| | | acgatgtccctatcagggactatcgcatcgatgagaagaactttgtggtcgtcatggtgaccaa | |
| | | gaccaaagccggccagggtacctcagcacccccagaggcctcacccacagctgccccaga | |
| | | gtcctctacatccttcccgcctgcccccacctcaggcatgtcccatcccccacctgccgccag | |
| | | agaggacaagagcccatcagaggaatccgcccccacgacgtccccagagtctgtgtcagg | |
| | | ctctgttccctcttcaggtagcagcgggcgagaggaagacgcggcctccacgctagtgacg | |
| | | ggctctgagtatgagacgatgctgacggagatcatgtccatgggctatgagcgagagcgggt | |
| | | cgtggccgccctgagagccagctacaacaacccccaccgagccgtggagtatctgctcacg | |
| | | ggaattcctgggagccccgagccggaacacggttctgtccaggagagccaggtatcggag | |
| | | cagccggccacggaagcaggagagaaccccctggagttcctgcgggaccagccccagttc | |
| | | cagaacatgcggcaggtgattcagcagaaccctgcgctgctgcccgccctgctccagcagct | |
| | | gggccaggagaaccctcagcttttacagcaaatcagccggcaccaggagcagttcatccag | |
| | | atgctgaacgagccccctggggagctggcggacatctcagatgtggagggggaggtgggc | |
| | | gccataggagaggaggccccgcagatgaactacatccaggtgacgccgcaggagaaaga | |
| | | agctatagagaggttgaaggccctgggcttcccagagagcctggtcatccaggcctatttcgc | |
| | | gtgtgaaaaaaatgagaacttggctgccaacttcctcctgagtcagaactttgatgacgagtga | |
| | | tgccaggaagccaggccaccgaagcccccaccctacccttattccatgaaagttttataaaag | |
| | | aaaaaatatatatatattcatgtttatttaagaaatggaaaaaaaaatcaaaaatcttaaaaaaaca | |
| | | agcaaacagtccagcttcctgtcctcctaaagtggcccctgttcccatctcccgggccagaca | |
| | | gctgtccccccgtcctcctccccagcccagcctgctcagagaagctggcaggactgggagg | |
| | | cgacagatgggcccctcttggcctctgtcccagctctctgcagccagacggaaaggcggctg | |
| | | cttgcctctccatcctccgaaaaacccctgaggacccccccccatcctcttctaggatgaggg | |
| | | gaagctggagccccaactttgatcctccattggagtggcccaaatctttccatctagggcaagt | |
| | | cctgaaaggcccaaggccccctccccagtctggccttggcctccagcctggagaagggcta | |
| | | acatcagctcattgtcaaggccacccccaccccagaacagaaccgtgtctctgataaaggtttt | |
| | | gaagtgaataaagttttaaaaactag | |
| REPIN1 | NM_013400.3 | cccggcggggggaggttggggacgggcctggcagttgtgaactcgaacctgccgctgtcg | 23 |
| | | ccgcggcggggcggggagcgagagtgggccgcggaggccggccttcgggctccatgga | |
| | | cgggcgccgcgtccctgcacagcccgccgcagaggtaaggctggcctctctgcagtcaga | |
| | | ggtctgagctctgccatggggataggggtgtctttattactgcagttttctctaacacctggggg | |
| | | ctaccggagtgtgggccgaagcaggcgctgcagccgcggaagtatccccaggaacatccc | |
| | | caagaggagctggaaaaagcctcatccccagctctgcagtctccaggggagctcagtgtctg | |
| | | tttgtccagcttctcagagttgctgtgcagctcggatgtggcataggaaacagcagacacagg | |
| | | gagagggcagcataaggcactgtagggagcagtggccacattttctgcagaggaagaacc | |
| | | gatgctggaacgtcgttgcaggggccccctggccatgggcctggcccagccccgactccttt | |
| | | ctgggccctcccaggagtcaccccagaccctggggaaggagtcccgcgggctgaggcaa | |
| | | caaggcacgtcagtggcccagtctggtgcccaagccccaggcagggcccatcgctgtgccc | |
| | | actgtcgaaggcacttccctggctgggtggctctgtggcttcacacccgccggtgccaggcc | |
| | | cggctgcccttgccctgccctgagtgtggccgtcgctttcgccatgcccccttcttagcactgc | |
| | | accgccaggtccatgctgctgccaccccagacctgggctttgcctgccacctctgtgggcag | |
| | | agcttccgaggctgggtggccctggttctgcatctgcgggcccattcagctgcaaagcggcc | |
| | | catcgcttgtcccaaatgcgagagacgcttctggcgacgaaagcagcttcgagctcatctgcg | |
| | | gcggtgccaccctcccgccccggaggcccggcccttcatatgcggcaactgtggccggag | |
| | | ctttgcccagtgggaccagctagttgcccacaagcgggtgcacgtagctgaggccctggag | |
| | | gaggccgcagccaaggctctggggccccggcccaggggccgccccgcggtgaccgccc | |
| | | cccggcccggtggagatgccgtcgaccgccccttccagtgtgcctgttgtggcaagcgcttc | |
| | | cggcacaagcccaacttgatcgctcaccgccgcgtgcacacgggcgagcggccccaccag | |
| | | tgccccgagtgcgggaagcgctttaccaataagccctatctgacttcgcaccggcgcatccac | |
| | | accggcgagaagccctacccgtgcaaagagtgcggccgccgcttccggcacaaacccaac | |
| | | ctgctgtctcacagcaagattcacaagcgatccgaggggtcggcccaggccgcccccggcc | |
| | | cggggagcccccagctgccagccggcccccaggagtccgcggccgagcccaccccggc | |
| | | ggtacctctgaaaccggcccaggagccgccgccaggggccccgccagagcacccgcagg | |
| | | acccgatcgaagcccccccctccctctacagctgcgacgactgcggcaggagcttccggct | |
| | | ggagcgcttcctgcgggcccaccagcggcagcacaccggggagcggcccttcacctgcg | |
| | | ccgagtgcgggaagaacttcggcaagaagacgcacctggtggcgcactcgcgcgtgcact | |
| | | ccggcgagcggcccttcgcctgcgaggagtgcggccgccgcttctcccagggcagccatct | |
| | | ggcggcgcatcggcgcgaccacgcccccgatcggcccttcgtgtgtcccgactgcggcaa | |
| | | ggccttccgccacaaaccctacctggcggcgcaccggcgcatccacaccggcgagaagcc | |
| | | ctacgtctgccccgactgcggcaaagccttcagccagaagtccaacctggtgtcgcaccggc | |
| | | gcatccacacgggcgagcggccctacgcctgtcccgactgcgaccgcagcttcagccaga | |
| | | agtccaacctcatcacccaccgcaagagccacatccgggacggcgccttctgctgtgccatc | |
| | | tgtggccagaccttcgacgacgaggagagactcctggcccaccagaagaagcacgatgtct | |
| | | gagacggtgggcggggccgtgttggctgagagagggctggggtccttcgtggtgggagtc | |
| | | gcagtgggctgggggtgcctgcctagtgctggagtaggggacaatgggaatcctagaggg | |
| | | gatggaagacgcggggagtgagctgggtgggccctgctagcgagagaggtcaaccccgg | |
| | | tggccagggaacccacttccaagcgcagggacgccggcctccagctggtgtgtgctaaggc | |
| | | tccgtcctgactgccctgtgccctggaaaagcagcaatagcatccgccccttagagccctctg | |
| | | gctagaggagccaccagtggaaaggaagaccctccatcctctggtattaacgccttaatgccc | |
| | | ctgtcttttactgtaagttacttaagatcatttttggaagcaggcgtggtagagtcctgtaaatgaa | |
| | | tgctctgggctagatacagcttggagaacctgctggccttgttagacagcacttgggcctttgc | |
| | | cagcagcaagaggtgaagcgaagccactcttacctctcccttcccctcccacctgccccctgc | |
| | | gtaggcacccagacttggagagacccgtctgctgttaatacttccatcctcttccttcccaaaga | |
| | | gcagatcccaaggcatttactccttggtctgtctcgctttatctgtcgcccctcccagcgctgag | |
| | | agcctcccctggctgtcagcagcactgtgtccaggctcttgtctgaacaccgcagcccctcctt | |
| | | cgctccttccagagctcagcatgtcacggcaaggactgccgcattggtgatggagggccag | |
| | | ctgaggggaagttgctggtgagtttccttttctccatttctagcatatggacacctggcctctgctt | |
| | | gagcacttaggtgacaggaacttccgcacctcctgaggccctggatgattctaattgttagaaa | |
| | | ttctaattgttagaaatccttccttataatgaatgaattctgctttcctataatttctacctattgggcc | |
| | | ttgttctgttctctggaactaaacagaacaaccatttacccctccttttcaaactagagaataaag | |
| | | atttggttttagaactggtaaaaaaaaaaaaaaaaaa | |
| SDR39U1 | NM_020195.2 | actttacgcaggcgcgttaggtcctagtcgctatgcgtgtgcttgtgggtggcgggacaggctt | 24 |
| | | cattgggacagccctaacccagctgctgaatgccagaggccacgaagtgacgttggtctccc | |
| | | gaaagcccgggcccggccggatcacgtgggatgagctcgctgcatcggggctgccgagct | |
| | | gcgatgccgccgtcaacctggccggagagaacatcctcaaccctctccgaagatggaatga | |
| | | aaccttccaaaaagaggtaatcggcagccgcctagagaccacccaattgctggctaaagcca | |
| | | tcaccaaagccccacaaccccccaaggcctgggtcttagtcacaggtgtagcttactaccagc | |
| | | ccagtctgactgcggagtatgatgaagacagcccaggaggggactttgactttttctccaacct | |
| | | cgtaaccaaatgggaagctgcagccaggcttcctggagattctacacgccaggtggtggtgc | |
| | | gctcaggggttgtgctgggccgtgggggtggtgccatgggccacatgctgctgccctttcgc | |
| | | ctgggcctggggggccccatcggctcaggccaccaattcttcccctggatacacatcgggga | |
| | | cctggcaggaatcctgacccatgcccttgaagcaaaccacgtgcacggggtcctgaatgga | |
| | | gtggctccatcctccgccactaatgctgagtttgcccagaccttgggtgctgccctgggccgc | |
| | | cgagccttcatccctctccccagcgctgtggtgcaagctgtctttgggcgacagcgtgccatc | |
| | | atgctgctggagggccagaaggtgatcccacagcgaacactggccactggctaccagtattc | |
| | | cttcccagagctaggggctgccttaaaggaaattgtagcctaagtaggtcgtggcaagggcct | |
| | | gaggcctgttcctcacaggcttccaggttaggcactgtgaataggctcagctcctctagagag | |
| | | ctgaagccatctggttcttagattcctctcccagtcctctttcccattgttctgttgctccaccttatt | |
| | | gtctcaaggccgtaatctcatcaggttgggacattaatcttttcaactccttgtaagatttcccagt | |
| | | ttggtttctctacatgtcctgcagctgccccacttctcctttacgctgtgtagagaatgctctgcag | |
| | | tttaggcaataaaaataaattgtctcactaaaaaaaaaaaaaaaaaa | |
| SETBP1 | NM_001130110.1 | aagcgcggggccggccggggcgcccgccgctcgccgccgcctccgcgcgcccggggg | 25 |
| | | ccccggcgccccccgagctagggcggccagctcgcggctcgccgtttgacagatgctcatc | |
| | | gccatggagttgccgcagcagcacctttgggggctcgggcgagcgacgggagccgggatc | |
| | | tgagcgagcgccggggccagcggagccggagccgccgggacatggttgcagatctgatct | |
| | | cttctgaacacctcatcgtgtctccatccctgggaatctgaccctagcaactggaccactttgttc | |
| | | ttggaattttgggtgtcctcttttctcacctttcccttttcccttttccccttccccctcctgagaactc | |
| | | cggaagactgtagagattgtcatggagtccagggaaaccttaagcagctcccggcaaagag | |
| | | ggggcgagtcagacttcctgccggtctcctcagccaagcccccagctgctcctggctgtgca | |
| | | ggagaacctttgctctccactccaggacctgggaaggggatcccggtgggcggagagcgc | |
| | | atggagccagaggaggaggatgaactaggctcagggcgggatgtggattccaactccaac | |
| | | gcggacagtgagaaatgggtggcaggagatggtttggaagagcaggaattttctatcaagga | |
| | | ggcaaacttcacagagggaagtctgaagctaaagattcagaccacaaagcgggctaagaaa | |
| | | cccccaaagaatttggagaactatatatgtccacctgagatcaagatcaccatcaagcagtctg | |
| | | gggaccagaaggtgtcccgtgctggaaaaaatagcaaagccacgaaggaggaagaaaga | |
| | | agccactccaaaaagaagctcctcacagccagtgaccttgcagccagtgacctcaaaggattt | |
| | | cagccacagattaaagactccagtaaggaggaagtctggaagagaagaggaggccaaggc | |
| | | atcccattcaaaaagcaattcctgtcccaggaacgtgccatgtgcttctcatgcccccggaacc | |
| | | cattccccgcaaaacccggttctctcactcttccttttcacagtgaacctgcagtctgggcacaa | |
| | | gaagtataacttcgcatggattctgcaaagcccacacctgtggtcattccctgttctttccattca | |
| | | acaatggagacttgcccaagattgtaaactagtgagtgacagcatttgggcttatgatcttctct | |
| | | gcctgctagctagacattctctctgggtctaaaagataatccaaaaagatccagcttcacaatgc | |
| | | tgccctgaagagataatgcattaggcggccctgatgcagcattcactgtcttccagggcaggc | |
| | | ttgatcccatgagtttgcttgctcagacgatcacttaggaaacacatgcctttactctctaggcctt | |
| | | ttttctagcttgccttgtatcagctatgccatggatctttgctctcttaccccatgctatacagagtat | |
| | | gggctccaagccacagctggcctgtcaagtgtgtgtcgctggtccaccatgggatacatttag | |
| | | aaacttttatagcaatttgacatttttgtgatatccaagcatgtgattgttttcctacggatttgtctta | |
| | | tagtattttaccaaagtttccacacaaaaagtatggattaaggacaaagtatctggtccttcatca | |
| | | aagatcgtttgataagctctgttctagttaaccaacactgagctttcctagttttaataaaagagta | |
| | | ggatttggaaaaaaaaaaaaaaaaaaaa | |
| SLC14A1 | NM_001128588.3 | acacagagcagagtggggctctgagtatataactgttaggtgcctccctccagcaccatctcct | 26 |
| | | gagaagcactctcccttgtcgtggaggtgggcaaatctttatcagccactgccttctgctgcca | |
| | | ggaagccagctagagtggtctttaaagaaaactgggcatctcctgctacttaaaatcaaaaact | |
| | | acctaaaataaagattataaaaaagtaaggatgaatggacggtctttgattggcggcgctggtg | |
| | | acgcccgtcatggtcctgtttggaaggacccttttggaactaaagctggtgacgcagcgcgca | |
| | | gaggcatcgcccggctaagcttggccctggcagatgggtcgcaggaacaggagccagag | |
| | | gaagagatagccatggaggacagccccactatggttagagtggacagccccactatggttag | |
| | | gggtgaaaaccaggtttcgccatgtcaagggagaaggtgcttccccaaagctcttggctatgt | |
| | | caccggtgacatgaaagaacttgccaaccagcttaaagacaaacccgtggtgctccagttcat | |
| | | tgactggattctccggggcatatcccaagtggtgttcgtcaacaaccccgtcagtggaatcctg | |
| | | attctggtaggacttcttgttcagaacccctggtgggctctcactggctggctgggaacagtgg | |
| | | tctccactctgatggccctcttgctcagccaggacaggtcattaatagcatctgggctctatggc | |
| | | tacaatgccaccctggtgggagtactcatggctgtcttttcggacaagggagactatttctggtg | |
| | | gctgttactccctgtatgtgctatgtccatgacttgcccaattttctcaagtgcattgaattccatgc | |
| | | tcagcaaatgggacctccccgtcttcaccctccctttcaacatggcgttgtcaatgtacctttcag | |
| | | ccacaggacattacaatccattctttccagccaaactggtcatacctataactacagctccaaat | |
| | | atctcctggtctgacctcagtgccctggagttgttgaaatctataccagtgggagttggtcagat | |
| | | ctatggctgtgataatccatggacagggggcattttcctgggagccatcctactctcctcccca | |
| | | ctcatgtgcctgcatgctgccataggatcattgctgggcatagcagcgggactcagtctttcag | |
| | | ccccatttgaggacatctactttggactctggggtttcaacagctctctggcctgcattgcaatg | |
| | | ggaggaatgttcatggcgctcacctggcaaacccacctcctggctcttggctgtgccctgttca | |
| | | cggcctatcttggagtcggcatggcaaactttatggctgaggttggattgccagcttgtacctg | |
| | | gcccttctgtttggccacgctattgttcctcatcatgaccacaaaaaattccaacatctacaagat | |
| | | gcccctcagtaaagttacttatcctgaagaaaaccgcatcttctacctgcaagccaagaaaaga | |
| | | atggtggaaagccctttgtgagaacaagccccatttgcagccatggtcacgagtcatttctgcc | |
| | | tgactgctccagctaacttccagggtctcagcaaactgctgtttttcacgagtatcaactttcata | |
| | | ctgacgcgtctgtaatctgttcttatgctcattttgtattttcctttcaactccaggaatatccttgag | |
| | | catatgagagtcacatccaggtgatgtgctctggtatggaatttgaaaccccaatggggccttg | |
| | | gcactaagactggaatgtatataaagtcaaagtgctccaacagaaggaggaagtgaaaacaa | |
| | | actattagtatttattgatattcttggtgtttagctggctcgatgatgttaacagtattaaaaattaaa | |
| | | ccccataaaccaactaagccttatggaattcacagtcacaaaatcgaagttaatccagaattctg | |
| | | tgataagcagcttggctttttttttaaatcaatgcaagttacacattatagccagaatctgtatcaca | |
| | | gaggtgcaagctgacagcagagctcagtccccacttcctgcaaacaatggcctgcaccctat | |
| | | cccttgtgtgtgtgacattctctcatgggacaatgttggggtttttcagactgacaggactgcaa | |
| | | gagggagaaaggaattttgtcaatcaaaattattctgtattgcaacttttctcagagattgcaaag | |
| | | gattttttaggtagagattatttttccttatgaaaaatgatctgttttaaatgagataaaataggaga | |
| | | agttcctggcttaacctgttcttacatattaaagaaaagttacttactgtatttatgaaatactcagc | |
| | | ttaggcatttttactttaacccctaaattgattttgtaaatgccacaaatgcatagaattgttaccaa | |
| | | cctccaaagggctctttaaaatcatattttttattcatttgaggatgtcttataaagactgaaggca | |
| | | aaggtcagattgcttacgggtgttatttttataagttgttgaattccttaatttaaaaaagctcattatt | |
| | | ttttgcacactcacaatattctctctcagaaatcaatggcatttgaaccaccaaaaagaaataaa | |
| | | gggctgagtgcggtggctcacgcctgtaatcccagcactttggggagcccaggcgggcaga | |
| | | ttgcttgaacccaggagttcaagaccagcctgggcagcatggtgaaaccctgtatctacaaaa | |
| | | aatacaaaaattagccaggcatggtggtgggtgcctgtagttccagctacttgggaggctgag | |
| | | gtgggaaaatgacttgagcccaggaggaggaggctgcagtgagctaagattgcaccactgc | |
| | | actccaacctgggcgacaagagtgaaactgtgtctctcaaaaaaaaaaaaaaacaaacaaaa | |
| | | acaaaaacaaaacaaaacaaaacaaaacaaaacaggtaaggattcccctgttttcctctcttta | |
| | | attttaaagttatcagttccgtaaagtctctgtaaccaaacatactgaagacagcaacagaagtc | |
| | | acgttcagggactggctcacacctgtaatcccagcactttgggagatggaggtaaaaggatct | |
| | | cttgagcccaggagttcaagaccagcttgggcaacatagcaagactccatctcttaaaaaata | |
| | | aaaatagtaacattagccaggtgtagcagcacacatctgcagcagctactcaggaggctgag | |
| | | gtggaaagatcgcttgtgcacagaagttcgaggctgcagtgagctatatgatcatgtcactgc | |
| | | actccagcctgtgtgaccgagcaagaccctatctcaaaaaaattaattaattaattaattaattaat | |
| | | ttaaaaaggaagtcatgttcatttactttccacttcagtgtgtatcgtgtagtattttggaggttgga | |
| | | aagtgaaacgtaggaatcctgaagattttttccacttctagtttgcagtgctcagtgcacaatata | |
| | | cattttgctgaatgaataaacagaaatagggaagtaaacctacaaatattttagggagaagctc | |
| | | acttcttccttttctcaggaaaccaagcaagcaaacatatcgttccaattttaaaacccagtgacc | |
| | | aaagcctttggaactatgaatttgcaactgtcataggtttatggatattgctgtggagaagctcaa | |
| | | ttttcagtgtttgaactgaaccctttcttgttagggaacgtgtgaaagaagaattgtggggaaaa | |
| | | aaaagcaagcataaccaaagatcatcagcagtgaagaatctaggctgtggctgagagaacc | |
| | | agaggcctctaaaatggacccgagtcgatcttcagaacagggatctaccatgcaggagcttct | |
| | | tgtgctcacacaaatctgtaaatgggaacattgtacattgtcgaatttaaatgatattaattttctca | |
| | | agctatttttgttactattttcctaaaattgaatatttgcagggagcacttatactttttcctaatgtctg | |
| | | tataacaaatttctatgcaagtacatgaataaattatgctcacagctca | |
| SLC18A2 | NM_003054.4 | agagccggacggggtaaactgagcggcggcggcggggcgctggggcggagactgcgac | 27 |
| | | ccggagccgcccggactgacggagcccactgcggtgcgggcgttggcgcgggcacgga | |
| | | ggacccgggcaggcatcgcaagcgaccccgagcggagccccggagccatggccctgag | |
| | | cgagctggcgctggtccgctggctgcaggagagccgccgctcgcggaagctcatcctgttc | |
| | | atcgtgttcctggcgctgctgctggacaacatgctgctcactgtcgtggtccccatcatcccaa | |
| | | gttatctgtacagcattaagcatgagaagaatgctacagaaatccagacggccaggccagtg | |
| | | cacactgcctccatctcagacagcttccagagcatcttctcctattatgataactcgactatggtc | |
| | | accgggaatgctaccagagacctgacacttcatcagaccgccacacagcacatggtgacca | |
| | | acgcgtccgctgttccttccgactgtcccagtgaagacaaagacctcctgaatgaaaacgtgc | |
| | | aagttggtctgttgtttgcctcgaaagccaccgtccagctcatcaccaaccctttcataggacta | |
| | | ctgaccaacagaattggctatccaattcccatatttgcgggattctgcatcatgtttgtctcaaca | |
| | | attatgtttgccttctccagcagctatgccttcctgctgattgccaggtcgctgcagggcatcgg | |
| | | ctcgtcctgctcctctgtggctgggatgggcatgcttgccagtgtctacacagatgatgaagag | |
| | | agaggcaacgtcatgggaatcgccttgggaggcctggccatgggggtcttagtgggccccc | |
| | | ccttcgggagtgtgctctatgagtttgtggggaagacggctccgttcctggtgctggccgccct | |
| | | ggtactcttggatggagctattcagctctttgtgctccagccgtcccgggtgcagccagagagt | |
| | | cagaaggggacacccctaaccacgctgctgaaggacccgtacatcctcattgctgcaggctc | |
| | | catctgctttgcaaacatgggcatcgccatgctggagccagccctgcccatctggatgatgga | |
| | | gaccatgtgttcccgaaagtggcagctgggcgttgccttcttgccagctagtatctcttatctcat | |
| | | tggaaccaatatttttgggatacttgcacacaaaatggggaggtggctttgtgctcttctgggaa | |
| | | tgataattgttggagtcagcattttatgtattccatttgcaaaaaacatttatggactcatagctccg | |
| | | aactttggagttggttttgcaattggaatggtggattcgtcaatgatgcctatcatgggctacctc | |
| | | gtagacctgcggcacgtgtccgtctatgggagtgtgtacgccattgcggatgtggcattttgtat | |
| | | ggggtatgctataggtccttctgctggtggtgctattgcaaaggcaattggatttccatggctcat | |
| | | gacaattattgggataattgatattctttttgcccctctctgcttttttcttcgaagtccacctgccaa | |
| | | agaagaaaaaatggctattctcatggatcacaactgccctattaaaacaaaaatgtacactcag | |
| | | aataatatccagtcatatccgataggtgaagatgaagaatctgaaagtgactgagatgagatcc | |
| | | tcaaaaatcatcaaagtgtttaattgtataaaacagtgtttccagtgacacaactcatccagaact | |
| | | gtcttagtcataccatccatccctggtgaaagagtaaaaccaaaggttattatttcctttccatggt | |
| | | tatggtcgattgccaacagccttataaagaaaaagaagcttttctaggggtttgtataaatagtgt | |
| | | tgaaactttattttatgtatttaattttattaaatatcatacaatatattttgatgaaataggtattgtgta | |
| | | aatctataaatatttgaatccaaaccaaatataattttttaacttacattaacaaacatttgggcaaa | |
| | | aatcatattggtaatgagtgtttaaaattaaagcacacattatctctgagactcttccaacaaaga | |
| | | gaaactagaatgaagtctgaaaaacagaatcaagtaagacagcatgttatatagtgacactga | |
| | | atgttatttaacttgtagttactatcaatatatttatgcgttaaacagctagttctctcaagtgtagag | |
| | | gacaagaacttgtgtcagttatcttttgaatccataaatcttagctggcattagttttctatgtaatca | |
| | | cctacctagagagagttgtaaattatatgttaacatgttatctggttggcagcaaacactaaagc | |
| | | caataaaggaaaaacagtaaatgttccgaaagcagagaaaagcaaccaaacatattgttatga | |
| | | actaaaagctttccctttaagatgcatacttgtcttactggatgaagaaaattgagggtacatgta | |
| | | ccttatactgtcaaggttgtttaaacatgataaggttaatcgccatctacttcaagttttagaaaag | |
| | | gaaacaagaagctgaaaacagctgctctgactttaatatctgactatatctttgatctgtttgcag | |
| | | gtcatccaagtgttttctaggaatatatttattttaggttgtctgaaactactattttttagactcctga | |
| | | aagttgttcacatcaatgtgaagacaaattttaaatgaaaatgaagaatgaaattatgtcttgaat | |
| | | catatattaagaagtaaaaataatagtgatcaggcagaaaagaaaaatggaacatctaaaaat | |
| | | gtatgtgctaactatatcatccagtgtgcagtgttgtgtatttttctaagcatgacaacattgatgtg | |
| | | ccttttcagtgtaacagcaaatactgttagtgaacattgtcaatttatgtcattttgttaagagatat | |
| | | gactggagtgtgcagtgtggaatgtctctaatactacttgtgaatcctgcagttctataatcataa | |
| | | acaaaaattacttagtttcgttaagctaagattgtgtttgtgttaacttcgacatcaaggagcaaa | |
| | | gaactttagaacagactcctcaatcttgtgactttcttattctctaggaaagtaacacttcgtttcat | |
| | | gaagcttttctgtggggcttcgattatttcaagtctggtttctaagtgcagtgtgtttgaagcaaac | |
| | | gaacttccaactcacttatttggcattgggcaacttggccaagtctgccactttggaagatggct | |
| | | ctggaggaaactctcatatggctaaaaaggcaggctagtttcttacttctacaggggtagagcc | |
| | | ttaaaaaagaacgtgctacaaattggttctctttgagggtttctggttctccctgcccccaatacc | |
| | | atatactttattgcaattttatttttgcctttacggctctgtgtctttctgcaagaaggcctggcaaag | |
| | | gtatgcctgctgttggtccctcgggataagataaaatataaataaaaccttcagaactgttttgga | |
| | | gcaaaagatagcttgtacttggggaaaaaaattctaagttcttttatatgactaatattcttggttag | |
| | | caagactggaaagaggtgtttttttaaaatgtacataccagaacaaagaacatacagctctctg | |
| | | aacatttattttttgaacagaggtggtttttatgtttggacctggtaatacagatacaaaaactttaa | |
| | | tgaggtagcaatgaatattcaactgtttgactgctaagtgtatctgtccatattttagcaagtttact | |
| | | taataaatcttctgaaccatgaaaaaaaaaaaaa | |
| SMC4 | NM_001002800.2 | gccctcttctgaagaggcgtttctggaccactgagccccgcctcccactgtgagcggaaccct | 28 |
| | | accgtttttaaaaaaatctttttcaaaacttgccaggttgtctttccaaatatttttaataatagtgctg | |
| | | ctgctgtagaccacagagaaaagaatccctcgctcttccttttcacttagtagaaacttctaccg | |
| | | cgtaggtcccgccaggagttcgcgcatgcgcaggagcgacaataagatggcggtgataatc | |
| | | gccgcactttttttcaaattagtggatcccagaaatcattgcgcgcatttgtaacgaatttccgttc | |
| | | gagtttgtattttaggcgccattttcgagtgaaggacccggagccgaaacaccggtaggagc | |
| | | ggggaggtgggtactacacaaccgtctccagccttggtctgagtggactgtcctgcagcgac | |
| | | catgccccgtaaaggcacccagccctccactgcccggcgcagagaggaagggccgccgc | |
| | | cgccgtcccctgacggcgccagcagcgacgcggagcctgagccgccgtccggccgcacg | |
| | | gagagcccagccaccgccgcagagactgcaagtgaggaacttgataatagaagtttagaag | |
| | | agattttgaacagcattcctcctcccccgcctccagcaatgaccaatgaagctggagctcctcg | |
| | | gcttatgataactcatattgtaaaccagaacttcaaatcctatgctggggagaaaattctgggac | |
| | | ctttccataagcgcttttcctgtattatcgggccaaatggcagtggcaaatccaatgttattgattc | |
| | | tatgctttttgtgtttggctatcgagcacaaaaaataagatctaaaaaactctcagtattaatacat | |
| | | aattctgatgaacacaaggacattcagagttgtacagtagaagttcattttcaaaagataattgat | |
| | | aaggaaggggatgattatgaagtcattcctaacagtaatttctatgtatccagaacggcctgca | |
| | | gagataatacttctgtctatcacataagtggaaagaaaaagacatttaaggatgttggaaatctt | |
| | | cttcgaagccatggaattgacttggaccataatagatttttaattttacagggtgaagttgaacaa | |
| | | attgctatgatgaaaccaaaaggccagactgaacacgatgagggtatgcttgaatatttagaa | |
| | | gatataattggttgtggacggctaaatgaacctattaaagtcttgtgtcggagagttgaaatatta | |
| | | aatgaacacagaggagagaagttaaacagggtaaagatggtggaaaaggaaaaggatgcc | |
| | | ttagaaggagagaaaaacatagctatcgaatttcttaccttggaaaatgaaatatttagaaaaaa | |
| | | gaatcatgtttgtcaatattatatttatgagttgcagaaacgaattgctgaaatggaaactcaaaa | |
| | | ggaaaaaattcatgaagataccaaagaaattaatgagaagagcaatatactatcaaatgaaat | |
| | | gaaagctaagaataaagatgtaaaagatacagaaaagaaactgaataaaattacaaaatttatt | |
| | | gaggagaataaagaaaaatttacacagctagatttggaagatgttcaagttagagaaaagttaa | |
| | | aacatgccacgagtaaagccaaaaaactggagaaacaacttcaaaaagataaagaaaaggtt | |
| | | gaagaatttaaaagtatacctgccaagagtaacaatatcattaatgaaacaacaaccagaaac | |
| | | aatgccctcgagaaggaaaaagagaaagaagaaaaaaaattaaaggaagttatggatagcc | |
| | | ttaaacaggaaacacaagggcttcagaaagaaaaagaaagtcgagagaaagaacttatggg | |
| | | tttcagcaaatcggtaaatgaagcacgttcaaagatggatgtagcccagtcagaacttgatatc | |
| | | tatctcagtcgtcataatactgcagtgtctcaattaactaaggctaaggaagctctaattgcagct | |
| | | tctgagactctcaaagaaaggaaagctgcaatcagagatatagaaggaaaactccctcaaact | |
| | | gaacaagaattaaaggagaaagaaaaagaacttcaaaaacttacacaagaagaaacaaactt | |
| | | taaaagtttggttcatgatctctttcaaaaagttgaagaagcaaagagctcattagcaatgaatc | |
| | | gaagtagggggaaagtccttgatgcaataattcaagaaaaaaaatctggcaggattccagga | |
| | | atatatggaagattgggggacttaggagccattgatgaaaaatacgacgtggctatatcatcct | |
| | | gttgtcatgcactggactacattgttgttgattctattgatatagcccaagaatgtgtaaacttcctt | |
| | | aaaagacaaaatattggagttgcaacctttataggtttagataagatggctgtatgggcgaaaa | |
| | | agatgaccgaaattcaaactcctgaaaatactcctcgtttatttgatttagtaaaagtaaaagatg | |
| | | agaaaattcgccaagctttttattttgctttacgagataccttagtagctgacaacttggatcaagc | |
| | | cacaagagtagcatatcaaaaagatagaagatggagagtggtaactttacagggacaaatcat | |
| | | agaacagtcaggtacaatgactggtggtggaagcaaagtaatgaaaggaagaatgggttcct | |
| | | cacttgttattgaaatctctgaagaagaggtaaacaaaatggaatcacagttgcaaaacgactc | |
| | | taaaaaagcaatgcaaatccaagaacagaaagtacaacttgaagaaagagtagttaagttacg | |
| | | gcatagtgaacgagaaatgaggaacacactagaaaaatttactgcaagcatccagcgtttaat | |
| | | agagcaagaagaatatttgaatgtccaagttaaggaacttgaagctaatgtacttgctacagcc | |
| | | cctgacaaaaaaaagcagaaattgctagaagaaaacgttagtgctttcaaaacagaatatgat | |
| | | gctgtggctgagaaagctggtaaagtagaagctgaggttaaacgcttacacaataccatcgta | |
| | | gaaatcaataatcataaactcaaggcccaacaagacaaacttgataaaataaataagcaattag | |
| | | atgaatgtgcttctgctattactaaagcccaagtagcaatcaagactgctgacagaaaccttcaa | |
| | | aaggcacaagactctgtcttgcgtacagagaaagaaataaaagatactgagaaagaggtgga | |
| | | tgacctaacagcagagctgaaaagtcttgaggacaaagcagcagaggtcgtaaagaataca | |
| | | aatgctgcagaggaatccttaccagagatccagaaagaacatcgcaatctgcttcaagaatta | |
| | | aaagttattcaagaaaatgaacatgctcttcaaaaagatgcacttagtattaagttgaaacttgaa | |
| | | caaatagatggtcacattgctgaacataattctaaaataaaatattggcacaaagagatttcaaa | |
| | | aatatcactgcatcctatagaagataatcctattgaagagatttcggttctaagcccagaggatct | |
| | | tgaagcgatcaagaatccagattctataacaaatcaaattgcacttttggaagcccggtgtcatg | |
| | | aaatgaaaccaaacctcggtgccatcgcagagtataaaaagaaggaagaattgtatttgcaac | |
| | | gggtagcagaattggacaaaattacttatgaaagagacagttttagacaggcatatgaagatct | |
| | | tcggaaacaaaggcttaatgaatttatggcaggtttttatataataacaaataaattaaaggaaaa | |
| | | ttaccaaatgcttactttgggaggggacgccgaactcgagcttgtagacagcttggatcctttct | |
| | | ctgaaggaatcatgttcagtgttcgaccacctaagaaaagttggaaaaagatcttcaacctttcg | |
| | | ggaggagagaaaacacttagttcattggctttagtatttgctcttcaccactacaagcccactcc | |
| | | cctttacttcatggatgagattgatgcagcccttgattttaaaaatgtgtccattgttgcattttatat | |
| | | atatgaacaaacaaaaaatgcacagttcataataatttctcttcgaaataatatgtttgagatttcg | |
| | | gatagacttattggaatttacaagacatacaacataacaaaaagtgttgctgtaaatccaaaaga | |
| | | aattgcatctaagggactttgttgaactttatgctgaagattcttcaagttgattcagtgtattactg | |
| | | atttttttctatttgtaaaggattatgagttgtataaaatacatactccctaaactagatcatgaaact | |
| | | ggtttctgttttatgcagttgtcatttgtaaagtctaataaaatattctctataattgcttctagattaca | |
| | | aaaatatgacaatcttgtaagtagcagactatggagaaaaatgagttacctggagggtcaggt | |
| | | aacttgccaaactaaaaagtatgttagttgaggcaaagtcctaagcaaggttgtgctatcaagg | |
| | | ctcagcataccttcgtgggcctttgatttaccaacactggaaatgcctgccaactaatcttggata | |
| | | gattctttaaggcattccacttagcttgccagttgagacaatcaccacagttattacccaaatact | |
| | | atgaacatatttttgtaaaccagtcattctgaattatagtgatgagaatttaaatatatgcttttctag | |
| | | aatttgatgtttgaccatttatgacttaattaccagagagccagtaaattaggacagtgtttcaaca | |
| | | agcctaggctatctcgtaagttgaaaaatatcccactatagttgcttcatgagtatgaagtaagat | |
| | | ggcctctgatttacactggttcaatttacaaattttcaactttatgataggtttatccgggtactaaat | |
| | | gcatttcaacttgatagtttcaacttatgataggtttaccaggatgtagtcccactgttgaggagc | |
| | | atctatttaggggttaattactttagtaataagtggaaagtaagataccttgagtaatgtttgcctat | |
| | | aaaattgtcagcgtatttttacactattggctcaagaatgttataatgctaagggacataagttgg | |
| | | caaccacttggtttttggaaggactttcggtattgtattagaagtctgccctagctgttaaatttctg | |
| | | ggtatttatcctaaggaattaattaaagagttaattgttcctttcttcagtgggccattgttttagata | |
| | | tttaaaaaatccaacagtttctatcataatgtaactgtaaaaatgtaaacacattattagcatggac | |
| | | ttttaaataaagatttaaagaaagcaagatcggaaaaaaaaaaaaaaaaaa | |
| SPARC | NM_001309443.1 | gggagaaggaggaggccgggggaaggaggagacaggaggaggagggaccacggggt | 29 |
| | | ggaggggagatagacccagcccagagctctgagtggtttcctgttgcctgtctctaaacccct | |
| | | ccacattcccgcggtccttcagactgcccggagagcgcgctctgcctgccgcctgcctgcct | |
| | | gccactgagggttcccagcaccatgagggcctggatcttctttctcctttgcctggccgggag | |
| | | ggccttggcagcccctcaagaagccctgcctgatgagacagaggtggtggaagaaactgtg | |
| | | gcagaggtgactgaggtatctgtgggagctaatcctgtccaggtggaagtaggagaatttgat | |
| | | gatggtgcagaggaaaccgaagaggaggtggtggcggaaaatccctgccagaaccacca | |
| | | ctgcaaacacggcaaggtgtgcgagctggatgagaacaacacccccatgtgcgtgtgccag | |
| | | gaccccaccagctgcccagcccccattggcgagtttgagaaggtgtgcagcaatgacaaca | |
| | | agaccttcgactcttcctgccacttctttgccacaaagtgcaccctggagggcaccaagaagg | |
| | | gccacaagctccacctggactacatcgggccttgcaaatacatccccccttgcctggactctg | |
| | | agctgaccgaattccccctgcgcatgcgggactggctcaagaacgtcctggtcaccctgtatg | |
| | | agagggatgaggacaacaaccttctgactgagaagcagaagctgcgggtgaagaagatcc | |
| | | atgagaatgagaagcgcctggaggcaggagaccaccccgtggagctgctggcccgggact | |
| | | tcgagaagaactataacatgtacatcttccctgtacactggcagttcggccagctggaccagc | |
| | | accccattgacgggtacctctcccacaccgagctggctccactgcgtgctcccctcatcccca | |
| | | tggagcattgcaccacccgctttttcgagacctgtgacctggacaatgacaagtacatcgccct | |
| | | ggatgagtgggccggctgcttcggcatcaagcagaaggatatcgacaaggatcttgtgatct | |
| | | aaatccactccttccacagtaccggattctctctttaaccctccccttcgtgtttcccccaatgttta | |
| | | aaatgtttggatggtttgttgttctgcctggagacaaggtgctaacatagatttaagtgaatacatt | |
| | | aacggtgctaaaaatgaaaattctaacccaagacatgacattcttagctgtaacttaactattaag | |
| | | gccttttccacacgcattaatagtcccatttttctcttgccatttgtagctttgcccattgtcttattgg | |
| | | cacatgggtggacacggatctgctgggctctgccttaaacacacattgcagcttcaacttttctc | |
| | | tttagtgttctgtttgaaactaatacttaccgagtcagactttgtgttcatttcatttcagggtcttgg | |
| | | ctgcctgtgggcttccccaggtggcctggaggtgggcaaagggaagtaacagacacacgat | |
| | | gttgtcaaggatggttttgggactagaggctcagtggtgggagagatccctgcagaacccac | |
| | | caaccagaacgtggtttgcctgaggctgtaactgagagaaagattctggggctgtgttatgaa | |
| | | aatatagacattctcacataagcccagttcatcaccatttcctcctttacctttcagtgcagtttcttt | |
| | | tcacattaggctgttggttcaaacttttgggagcacggactgtcagttctctgggaagtggtcag | |
| | | cgcatcctgcagggcttctcctcctctgtcttttggagaaccagggctcttctcaggggctctag | |
| | | ggactgccaggctgtttcagccaggaaggccaaaatcaagagtgagatgtagaaagttgtaa | |
| | | aatagaaaaagtggagttggtgaatcggttgttctttcctcacatttggatgattgtcataaggttt | |
| | | ttagcatgttcctccttttcttcaccctccccttttttcttctattaatcaagagaaacttcaaagttaat | |
| | | gggatggtcggatctcacaggctgagaactcgttcacctccaagcatttcatgaaaaagctgc | |
| | | ttcttattaatcatacaaactctcaccatgatgtgaagagtttcacaaatccttcaaaataaaaagt | |
| | | aatgacttagaaactgccttcctgggtgatttgcatgtgtcttagtcttagtcaccttattatcctga | |
| | | cacaaaaacacatgagcatacatgtctacacatgactacacaaatgcaaacctttgcaaacac | |
| | | attatgcttttgcacacacacacctgtacacacacaccggcatgtttatacacagggagtgtatg | |
| | | gttcctgtaagcactaagttagctgttttcatttaatgacctgtggtttaacccttttgatcactacca | |
| | | ccattatcagcaccagactgagcagctatatccttttattaatcatggtcattcattcattcattcatt | |
| | | cacaaaatatttatgatgtatttactctgcaccaggtcccatgccaagcactggggacacagtta | |
| | | tggcaaagtagacaaagcatttgttcatttggagcttagagtccaggaggaatacattagataat | |
| | | gacacaatcaaatataaattgcaagatgtcacaggtgtgatgaagggagagtaggagagacc | |
| | | atgagtatgtgtaacaggaggacacagcattattctagtgctgtactgttccgtacggcagcca | |
| | | ctacccacatgtaactttttaagatttaaatttaaattagttaacattcaaaacgcagctccccaatc | |
| | | acactagcaacatttcaagtgcttgagagccatgcatgattagtggttaccctattgaataggtc | |
| | | agaagtagaatcttttcatcatcacagaaagttctattggacagtgctcttctagatcatcataag | |
| | | actacagagcacttttcaaagctcatgcatgttcatcatgttagtgtcgtattttgagctggggtttt | |
| | | gagactccccttagagatagagaaacagacccaagaaatgtgctcaattgcaatgggccaca | |
| | | tacctagatctccagatgtcatttcccctctcttattttaagttatgttaagattactaaaacaataaa | |
| | | agctcctaaaaaatcaaactgtattctggtgttctcttctacacagtgggagggcgagcagtag | |
| | | gagagattggcccatttggtgctggccatttgaggaatgcaagcccagcactagtctcataatc | |
| | | tctaggaatctgtagagagaggaattgaagtaaatttcagcattggctcattcagtcattcggcg | |
| | | acattcatcaggtacctgcaatgtgttaggggatcttatgagtaggcagcgtgcgtgatccttgc | |
| | | tcccctggagctttctaacattctagcaggcagaccacacataaatttgcaatactgtttctgata | |
| | | aaaacgtgctgtaaaggaaataaagcagagaactatcatggaaaaaaaaaaaaaaaaaa | |
| SQLE | NM_003129.3 | gtctgggccgagcccgcccagctggctgagacgcgtggagcctggcggcgagtgggggc | 30 |
| | | gtgcgacggttactctggttactggggccgcgccgcgctggcgagagccgccgcccgcga | |
| | | gggatgctggtgaggaagccgtcgggagccgccgccgccatctgagggaggtaccctgga | |
| | | aaccaccttttatcggtggggaagtgcagtcgcggtgggcggctctgggggccagcgaaac | |
| | | gggaggcctctaaatctttaggttggggctgcattgccctggagccgcactcttgagtccgag | |
| | | gccatcttttgttggagaaggcgtcggcgttggcgttttcccgaggttgggctgtacagtgtctc | |
| | | cgtccgcggaaaaagaagcctctgaacccgcgccggcccgcagcccccgtgccttccggc | |
| | | cgctgctcgccgtcgccagaggctaggccacgtttcccccagtgccgaggtgtttctgtgacc | |
| | | ctccctccactcccattcccttctgaaagggcacctgctcttggtgagaaaagaaattatagcac | |
| | | gaagagccagtatcagaagagtatccatcacccgcagcaaccgctcagggaacaccatcaa | |
| | | aaaagaaaaaaagggaatatctggatttcctgggcgaggaggagcgagtctgctcgggagc | |
| | | tgttccagcaggcgatttttaaatactgctttctacgccctatacaacttggcttcacatacttttac | |
| | | actaactttatatgatttttaaaaactggtctgatcggacttctcgtcctgggacactgtttactgga | |
| | | gtctggccggctctccgtgctcctcttggtacctcattttggggagaaccttaaacccactcgag | |
| | | cagataatctccgccttgaccggtgccaccaaagaagccttggaaccatgtggacttttctgg | |
| | | gcattgccactttcacctatttttataagaagttcggggacttcatcactttggccaacagggag | |
| | | gtcctgttgtgcgtgctggtgttcctctcgctgggcctggtgctctcctaccgctgtcgccaccg | |
| | | aaacgggggtctcctcgggcgccagcagagcggctcccagttcgccctcttctcggatattct | |
| | | ctcaggcctgcctttcattggcttcttctgggccaaatccccccctgaatcagaaaataaggag | |
| | | cagctcgaggccaggaggcgcagaaaaggaaccaatatttcagaaacaagcttaataggaa | |
| | | cagctgcctgtacatcaacatcttctcagaatgacccagaagttatcatcgtgggagctggcgt | |
| | | gcttggctctgctttggcagctgtgctttccagagatggaagaaaggtgacagtcattgagaga | |
| | | gacttaaaagagcctgacagaatagttggagaattcctgcagccgggtggttatcatgttctca | |
| | | aagaccttggtcttggagatacagtggaaggtcttgatgcccaggttgtaaatggttacatgatt | |
| | | catgatcaggaaagcaaatcagaggttcagattccttaccctctgtcagaaaacaatcaagtgc | |
| | | agagtggaagagctttccatcacggaagattcatcatgagtctccggaaagcagctatggca | |
| | | gagcccaatgcaaagtttattgaaggtgttgtgttacagttattagaggaagatgatgttgtgatg | |
| | | ggagttcagtacaaggataaagagactggagatatcaaggaactccatgctccactgactgtt | |
| | | gttgcagatgggcttttctccaagttcaggaaaagcctggtctccaataaagtttctgtatcatct | |
| | | cattttgttggctttcttatgaagaatgcaccacagtttaaagcaaatcatgctgaacttattttagc | |
| | | taacccgagtccagttctcatctaccagatttcatccagtgaaactcgagtacttgttgacattag | |
| | | aggagaaatgccaaggaatttaagagaatacatggttgaaaaaatttacccacaaatacctgat | |
| | | cacctgaaagaaccattcttagaagccactgacaattctcatctgaggtccatgccagcaagct | |
| | | tccttcctccttcatcagtgaagaaacgaggtgttcttcttttgggagacgcatataatatgaggc | |
| | | atccacttactggtggaggaatgactgttgcttttaaagatataaaactatggagaaaactgcta | |
| | | aagggtatccctgacctttatgatgatgcagctattttcgaggccaaaaaatcattttactgggca | |
| | | agaaaaacatctcattcctttgtcgtgaatatccttgctcaggctctttatgaattattttctgccac | |
| | | agatgattccctgcatcaactaagaaaagcctgttttctttatttcaaacttggtggcgaatgtgtt | |
| | | gcgggtcctgttgggctgctttctgtattgtctcctaaccctctagttttaattggacacttctttgct | |
| | | gttgcaatctatgccgtgtatttttgctttaagtcagaaccttggattacaaaacctcgagcccttc | |
| | | tcagtagtggtgctgtattgtacaaagcgtgttctgtaatatttcctctaatttactcagaaatgaa | |
| | | gtatatggttcattaagcttaaaggggaaccatttgtgaatgaatatttggaacttaccaagtcct | |
| | | aagagacttttggaagaggatatatatagcatagtaccataccacttataaagtggaaactcttg | |
| | | gaccaagatttggattaatttgtttttgaagttttttgtatataaatatgtaaatacatgctttaatttgc | |
| | | aatttaaaatgaaggggttaaataagttagacatttaaaagaaatgattgttaccataaattagtg | |
| | | ctaatgctgaggagaactacagtttttcttttgaatttagtatttgagatgagttgttgggacatgc | |
| | | aaataaaatgaagaatgaa | |
| STRIP1 | NM_001270768.1 | attttacccagcccctgttcaagatggagttgctgtggttcacacatctctgacaaaaatacagg | 31 |
| | | gctattcggagtcaccagacctggagtttgagtatgctgacacagacaagtgggctgcagag | |
| | | ctctcggagctttacagctacacggaagggccagaattcctgatgaatcgaaaatgctttgag | |
| | | gaggacttccggatccatgtgacagacaagaagtggactgagctggataccaaccagcacc | |
| | | ggacccatgccatgaggctcctggatggcttggaagtcactgccagggagaagagactcaa | |
| | | ggtggctcgagcaattctctatgttgctcaaggcacgtttggggagtgcagctcggaggcaga | |
| | | ggtgcagtcctggatgcgctacaacatctttctcctcctggaggtgggcacgttcaatgctttgg | |
| | | tggagcttctgaacatggaaatagacaacagtgccgcctgcagcagtgctgtgaggaagcct | |
| | | gccatctccctggctgacagcacagacctcagggtcctgctcaacatcatgtacctgatagtg | |
| | | gagaccgttcatcaggagtgtgagggtgacaaggctgagtggaggaccatgcggcagacct | |
| | | tcagagccgagctgggctccccgctgtacaacaatgagccatttgccatcatgctgtttgggat | |
| | | ggtgaccaaattttgcagtggtcacgcccctcactttcccatgaagaaagttctcttgctgctctg | |
| | | gaagacagtattgtgcacgctaggcggctttgaggagctgcagagcatgaaggctgagaag | |
| | | cgcagcatcctgggcctccccccgcttcctgaggacagcatcaaagtgattcgcaacatgag | |
| | | agcagcctctccaccagcatctgcttcagacttgattgagcagcagcagaaacggggccgcc | |
| | | gagagcacaaggctctgataaagcaggacaacctagatgccttcaacgagcgggatcccta | |
| | | caaggctgatgactctcgagaagaggaagaggagaatgatgatgacaacagtctggaggg | |
| | | ggagacgtttcccctggaacgggatgaagtgatgcctcccccgctacagcacccacagactg | |
| | | acaggctgacttgccccaaagggctcccgtgggctcccaaggtcagagagaaagacattga | |
| | | gatgttccttgagtccagccgcagcaaatttataggttacactctaggcagtgacacgaacaca | |
| | | gtggtggggctgcccaggccaatccacgaaagcatcaagactctgaaacagcacaagtaca | |
| | | cgtcgattgcagaggtccaggcacagatggaggaggaatacctccgctcccctctctcaggg | |
| | | ggagaagaagaagttgagcaagtccctgcagaaaccctctaccaaggcttgctccccagcct | |
| | | gcctcagtatatgattgccctcctgaagatcctgttggctgcagcacccacctcaaaagccaaa | |
| | | acagactcaatcaacatcctagcggacgtcttgcctgaggagatgcccaccacagtgttgcag | |
| | | agcatgaagctgggggtggatgtaaaccgccacaaagaggtcattgttaaggccatttctgct | |
| | | gtcctgctgctgctgctcaagcactttaagttgaaccatgtctaccagtttgaatacatggccca | |
| | | gcacctggtgtttgccaactgcattcctttgatcctaaagttcttcaatcaaaacatcatgtcctac | |
| | | atcactgccaagaacagcatttctgtcctggattaccctcactgcgtggtgcatgagctgccag | |
| | | agctgacggcggagagtttggaagcaggtgacagtaaccaattttgctggaggaacctctttt | |
| | | cttgtatcaatctgcttcggatcttgaacaagctgacaaagtggaagcattcaaggacaatgat | |
| | | gctggtggtgttcaagtcagcccccatcttgaagcgggccctaaaggtgaaacaagccatga | |
| | | tgcagctctatgtgctgaagctgctcaaggtacagaccaaatacttggggcggcagtggcga | |
| | | aagagcaacatgaagaccatgtctgccatctaccagaaggtgcggcatcggctgaacgacg | |
| | | actgggcatacggcaatgatcttgatgcccggccttgggacttccaggcagaggagtgtgcc | |
| | | cttcgtgccaacattgaacgcttcaacgcccggcgctatgaccgggcccacagcaaccctga | |
| | | cttcctgccagtggacaactgcctgcagagtgtcctgggccaacgggtggacctccctgagg | |
| | | actttcagatgaactatgacctctggttagaaagggaggtcttctccaagcccatttcctgggaa | |
| | | gagctgctgcagtgaggctgttggttaggggactgaaatggagagaaaagatgatctgaagg | |
| | | tacctgtgggactgtcctagttcattgctgcagtgctcccatcccccaccaggtggcagcaca | |
| | | gccccactgtgtcttccgcagtctgtcctgggcttgggtgagcccagcttgacctccccttggtt | |
| | | cccagggtcctgctccgaagcagtcatctctgcctgagatccattcttcctttacttcccccacc | |
| | | ctcctctcttggatatggttggttttggctcatttcacaatcagcccaaggctgggaaagctgga | |
| | | atgggatgggaacccctccgccgtgcatctgaatttcaggggtcatgctgatgcctctcgaga | |
| | | catacaaatccttgctttgtcagcttgcaaaggaggagagtttaggattagggccagggccag | |
| | | aaagtcggtatcttggttgtgctctggggtgggggtggggtgtttctgatgttattccagcctcct | |
| | | gctacattatatccagaagtaattgcggaggctccttcagctgcctcagcactttgattttggaca | |
| | | gggacaaggtaggaagagaagcttcccttaaccagaggggccatttttccttttggctttcgag | |
| | | ggcctgtaaatatctatatataattctgtgtgtattctgtgtcatgttggggtttttaatgtgattgtgt | |
| | | attctgtttacattaaaaagaagcaaaaataaaaaaaaaaaaa | |
| STX12 | NM_177424.2 | gcacgcagccttagctccttccccgcccttgctcttcccagtttctccgtcagcctgcgggtccc | 32 |
| | | ggctggcggctgcttccggtaggagagcggtgtagagcgagcaggtctcagctcctcgtcat | |
| | | gtcatacggtcccttagacatgtaccggaacccggggccctcggggccccagctccgggac | |
| | | ttcagcagcatcatccagacgtgcagcggcaacatccagcggatcagccaagccactgctca | |
| | | gataaagaatttgatgagccagctaggaactaagcaggactcaagcaagctacaggaaaatc | |
| | | tgcaacagttacaacactccacaaatcagctcgccaaggaaacaaatgaattgctgaaagaat | |
| | | tagggtccttgccccttcccttatctacttcagaacagcgccagcagagacttcagaaggaac | |
| | | gcctcatgaatgacttctctgcagccttaaacaatttccaggctgtgcagagaagggtatctga | |
| | | aaaggaaaaggagagtattgccagagcaagagctggatctcgtctttctgcagaagagagg | |
| | | caaagagaggagcagctggtctcatttgacagccatgaggagtggaaccagatgcagagcc | |
| | | aggaggatgaggtggccatcactgagcaggatttggaacttattaaagaaagagaaacggc | |
| | | aattcggcagctggaggctgacattttggatgtcaatcagatatttaaagatttggccatgatgat | |
| | | ccatgaccagggtgatctgattgatagcatagaagccaatgtggaaagctcagaggtgcacg | |
| | | tcgaaagagccactgaacagttacagcgagctgcttactatcagaaaaaatctcgcaagaag | |
| | | atgtgtatcctggtgcttgtcctgtcagtgattattctaatcttgggacttattatctggctagtttata | |
| | | aaacgaagtgattgcctccgatcgttctcccgctgagctgttttcaagggcaagtgcttgttgaa | |
| | | gtcttgccagaacaaactgatcacaagaagacagcatatatcagaacgtcctgtaatcatttagt | |
| | | tagaaactaactactaactagtctttggaattcgtgacctatggagacagtaattatcaatttattg | |
| | | attctattgatttctcaaattaggaattaacttatgtggattttgcttcctcttgtattctgattgccctt | |
| | | catcccaagtgtttactgaaaattccattctagatattcttgttttgacaaatgacactacagtctcg | |
| | | taatattgtcttttatgtatatacaaaatttacctttttactagcatctgagatagagttactttctggta | |
| | | cccagtatattggagtctgtcagaaactctataataggccaccagtttttattatttaacatttttattt | |
| | | gaatttctaagaagcctattctctatctattttgaaagattttggcactatatttaattggaaggtaaa | |
| | | atattgtacatgtgatccagagtaaatgagaagtctctatctgagctggtcagttactggagtac | |
| | | atgttactaatctgggtttaaagtttacttcattatctgctagtgtcatccacagcagttcatcctcat | |
| | | ccacactaagccatcctgttagcttttaaaggaagttaatttaattaacattaatatactctatggg | |
| | | ctccctctcccacctgtctgcatagaaaggcagaattagacatagcatgctttggaaaagcaaa | |
| | | taggaattgttgggaatgatttaatcttgttgttgttgttgttgttgttcacttgtggttctacattcctg | |
| | | gtgaatgatgaatgttgctgtcaaagggctgccccctaccttataagggttgctgggcatttgaa | |
| | | ggcaggaagatttttaaagatagattgaggttggtttaaaattattcctgtaaaccaacaataaag | |
| | | caaagaagaggttcatttttgtaaataacactggtttcaaatagtgatgttagacttaacctaattt | |
| | | ataaacaagagattaatatctccatgcatagttttagacaaaaaaagatgtttcaataaaattact | |
| | | gtcttgtaatataaatgttgtccacttcccttttccacaggcctagaacagttaaagggaacataa | |
| | | tttgtttaggctcccacataaatgtgaatctggccaacaactttggttcatcctttagtgaattaga | |
| | | ggatttggctaccctgagtatatttatattcatttcttctgttctccttctgttattatacttaatcttcta | |
| | | aactaaactaatgtgaacagtagggaagcaagggcccaaatgcataagtttctttgcactgttg | |
| | | cacttacttaatacaaataaatgttttttaaagcttttgtagtatgtttttatgagttaacatcctaatgt | |
| | | ggtaggtattaggtaatgtgctgtcatgagaaaaattgagacttccaagaaaactggacacca | |
| | | ggtgagggttggtttggagacggaataggtgtagctgcctttccttgaaaaacagtgtgtagag | |
| | | atggctgagtgcaatggctcacacttgtaatcccaacactttggaaggctgaggcgggaggat | |
| | | cagtatatcggtacgtctgagcccaagagttcaagatcagcctgggcagtatagcaagaccc | |
| | | catctccattttttttttaatgattttttaattaaaaaaaagaacaacaggatagagctgttggggtg | |
| | | gcacagtggcccaaagagcagcttcagagataattccttggttctatgatccctgtttaactcca | |
| | | aattacagtcggacttggatacatcatttgtaacattgtaggaaagaaaaaagtcttggttgtga | |
| | | aaaacgatttgcatttgggtaaaataaagtgaccatgcttttgttctgtaatactgtgtgacctgtg | |
| | | gttgttgtaatggtgatcatggagagcaaatatgaacttggcctggattttaaatggcctagaatt | |
| | | tgtggtagttgccaaagaggttctcctaggtggtcttaataaacctattcacagaattctccttaa | |
| | | aaaaaaaaaaaaaaaaaa | |
| TMPRSS2_1 | DQ204772.1 | taggcgcgagctaagcaggaggcggaggcggaggcggagggcgaggggcggggagc | 33 |
| | | gccgcctggagcgcggcaggaagccttatcagttgtgagtgaggaccagtcgttgtttgagt | |
| | | gtgcctacggaacgccacacctggctaagacagagatgaccgcgtcctcctccagcgactat | |
| | | ggacagacttccaagatgagcccacgcgtccctcagcaggattggctgtctcaacccccagc | |
| | | cagggtcaccatcaaaatggaatgtaaccctagccaggtgaatggctcaag | |
| TMPRSS2_2 | OM_TMPRSS2- | gggaacaaaagctggagctccaccgcggtggcggccgctctagccctcaaggaactctcct | 34 |
| | ERG_T2E4_ | gatgaatgcagtgtggccaaaggcgggaagatggtgggcagcccagacaccgttgggatg | |
| | COSF28.0 | aactacggcagctacatggaggagaagcacatgccacccccaaacatgaccacgaacgag | |
| | | cgcagagttatcgtgccagcagatcctacgctatggagtacagaccatgtgcggcagtggct | |
| | | ggagtgggcggtgaaagaatatggccttccagacgtcaacatcttgttattccagaacatcgat | |
| | | gggaaggaactgtgcaagatgaccaaggacgacttccagaggctcacccccagctacaac | |
| | | gccgacatccttctctcacatctccactacctcagagagactcctcttccacatttgacttcagat | |
| | | gatgttgataaagccttacaaaactctccacggttaatgcatgctagaaacacagggggtgca | |
| | | gcttttattttcccaaatacttcagtatatcctgaagctacgcaaagaattacaactaggccagatt | |
| | | taccatatgagccccccaggagatcagcctggaccggtcacggccaccccacgccccagtc | |
| | | gaaagctgctcaaccatctccttccacagtgcccaaaactgaagaccagcgtcctcagttaga | |
| | | accttatcagattcttggaccaacaagtagccgccttgcaaatccaggcagtggccagatcca | |
| | | gctttggcagttcctcctggagctcctgtcggacagctccaactccagctgcatcacctggga | |
| | | aggcaccaacggggagttcaagatgacggatcccgacgaggtggcccggcgctggggag | |
| | | agcggaagagcaaacccaacatgaactacgataagctcagccgcgccctccgttactactat | |
| | | gacaagaacatcatgaccaaggtccatgggaagcgctacgcctacaagttcgacttccacgg | |
| | | gatcgcccaggccctccagccccaccccccggagtcatctctgtacaagtacccctcagacc | |
| | | tcccgtacatgggctcctatcacgcccacccacagaagatgaactttgtggcgccccaccctc | |
| | | cagccctccccgtgacatcttccagtttttttgctgccccaaacccatactggaattcaccaactg | |
| | | ggggtatataccccaacactaggctccccaccagccatatgccttctcatctgggcacttacta | |
| | | ctaaagacctggcggaggcttttcccatcagcgtgcattcaccagcccatcgccacaaactct | |
| | | atcggagaacatgaatcaaaagtgcctcaagaggaatgaaaaaagctttactggggctgggg | |
| | | aaggaagccggggaagagatccaaagactcttgggagggagttactgaagtcttactgaagt | |
| | | cttactacagaaatgaggaggatgctaaaaatgtcacgaatatggacatatcatctgtggactg | |
| | | accttgtaaaagacagtgtatgtagaagcatgaagtcttaaggacaaagtgccaaagaaagtg | |
| | | gtcttaagaaatgtataaactttagagtagagtttgaatcccactaatgcaaactgggatgaaac | |
| | | taaagcaatagaaacaacacagttttgacctaacataccgtttataatgccattttaaggaaaact | |
| | | acctgtatttaaaaatagaaacatatcaaaaacaagagaaaagacacgagagagactgtggc | |
| | | ccatcaacagacgttgatatgcaactgcatggcatgtgctgttttggttgaaatcaaatacattcc | |
| | | gtttgatggacagctgtcagctttctcaaactgtgaagatgacccaaagtttccaactcctttaca | |
| | | gtattaccgggactatgaactaaaaggtgggactgaggatgtgtatagagtgagcgtgtgatt | |
| | | gtagacagaggggtgaagaaggaggaggaagaggcagagaaggaggagaccagggct | |
| | | gggaaagaaacttctcaagcaatgaagactggactcaggacatttggggactgtgtacaatg | |
| | | agttatggagactcgagggttcatgcagtcagtgttataccaaacccagtgttaggagaaagg | |
| | | acacagcgtaatggagaaaggggaagtagtagaattcagaaacaaaaatgcgcatctctttct | |
| | | ttgtttgtcaaatgaaaattttaactggaattgtctgatatttaagagaaacattcaggacctcatc | |
| | | attatgtgggggctttgttctccacagggtcaggtaagagatggccttcttggctgccacaatca | |
| | | gaaatcacgcaggcattttgggtaggcggcctccagttttcctttgagtcgcgaacgctgtgcg | |
| | | tttgtcagaatgaagtatacaagtcaatgtttttccccctttttatataataattatataacttatgcatt | |
| | | tatacactacgagttgatctcggccagccaaagacacacgacaaaagagacaatcgatataat | |
| | | gtggccttgaattttaactctgtatgcttaatgtttacaatatgaagttattagttcttagaatgcaga | |
| | | atgtatgtaataaaataagcttggcctagcatggcaaatcagatttatacaggagtctgcatttgc | |
| | | actttttttagtgactaaagttgcttaatgaaaacatgtgctgaatgttgtggattttgtgttataattt | |
| | | actttgtccaggaacttgtgcaagggagagccaaggaaataggatgtttggcacccaaatgg | |
| | | cgtcagcctctccaggtccttcttgcctcccctcctgtcttttatttctagccccttttggaacagaa | |
| | | ggaccccgggtttcacattggagcctccatatttatgcctggaatggaaagaggcctatgaag | |
| | | ctggggttgtcattgagaaattctagttcagcacctggtcacaaatcacccttaattcctgctatg | |
| | | attaaaatacatttgttgaacagtgaacaagctaccactcgtaaggcaaactgtattattactggc | |
| | | gggcggatcccccgggctgcaggaattcgatatcaagcttatcgataccgtcga | |
| TRIM29 | NM_012101.3 | ctcctcacaggtgtgtctctagtcctcgtggttgcctgccccactccctgccgagacgcctgcc | 35 |
| | | agaaaggtcacctatcctgaaccccagcaagcctgaaacagctcagccaagcaccctgcga | |
| | | tggaagctgcagatgcctccaggagcaacgggtcgagcccagaagccagggatgcccgg | |
| | | agcccgtcgggccccagtggcagcctggagaatggcaccaaggctgacggcaaggatgc | |
| | | caagaccaccaacgggcacggcggggaggcagctgagggcaagagcctgggcagcgcc | |
| | | ctgaagccaggggaaggtaggagcgccctgttcgcgggcaatgagtggcggcgacccatc | |
| | | atccagtttgtcgagtccggggacgacaagaactccaactacttcagcatggactctatggaa | |
| | | ggcaagaggtcgccgtacgcagggctccagctgggggctgccaagaagccacccgttacc | |
| | | tttgccgaaaagggcgagctgcgcaagtccattttctcggagtcccggaagcccacggtgtc | |
| | | catcatggagcccggggagacccggcggaacagctacccccgggccgacacgggcctttt | |
| | | ttcacggtccaagtccggctccgaggaggtgctgtgcgactcctgcatcggcaacaagcaga | |
| | | aggcggtcaagtcctgcctggtgtgccaggcctccttctgcgagctgcatctcaagccccacc | |
| | | tggagggcgccgccttccgagaccaccagctgctcgagcccatccgggactttgaggcccg | |
| | | caagtgtcccgtgcatggcaagacgatggagctcttctgccagaccgaccagacctgcatct | |
| | | gctacctttgcatgttccaggagcacaagaatcatagcaccgtgacagtggaggaggccaag | |
| | | gccgagaaggagacggagctgtcattgcaaaaggagcagctgcagctcaagatcattgaga | |
| | | ttgaggatgaagctgagaagtggcagaaggagaaggaccgcatcaagagcttcaccaccaa | |
| | | tgagaaggccatcctggagcagaacttccgggacctggtgcgggacctggagaagcaaaa | |
| | | ggaggaagtgagggctgcgctggagcagcgggagcaggatgctgtggaccaagtgaagg | |
| | | tgatcatggatgctctggatgagagagccaaggtgctgcatgaggacaagcagacccggga | |
| | | gcagctgcatagcatcagcgactctgtgttgtttctgcaggaatttggtgcattgatgagcaatt | |
| | | actctctccccccacccctgcccacctatcatgtcctgctggagggggagggcctgggacag | |
| | | tcactaggcaacttcaaggacgacctgctcaatgtatgcatgcgccacgttgagaagatgtgc | |
| | | aaggcggacctgagccgtaacttcattgagaggaaccacatggagaacggtggtgaccatc | |
| | | gctatgtgaacaactacacgaacagcttcgggggtgagtggagtgcaccggacaccatgaa | |
| | | gagatactccatgtacctgacacccaaaggtggggtccggacatcataccagccctcgtctcc | |
| | | tggccgcttcaccaaggagaccacccagaagaatttcaacaatctctatggcaccaaaggtaa | |
| | | ctacacctcccgggtctgggagtactcctccagcattcagaactctgacaatgacctgcccgtc | |
| | | gtccaaggcagctcctccttctccctgaaaggctatccctccctcatgcggagccaaagcccc | |
| | | aaggcccagccccagacttggaaatctggcaagcagactatgctgtctcactaccggccattc | |
| | | tacgtcaacaaaggcaacgggattgggtccaacgaagccccatgagctcctggcggaagga | |
| | | acgaggcgccacacccctgctcttcctcctgaccctgctgctcttgccttctaagctactgtgctt | |
| | | gtctgggtgggagggagcctggtcctgcacctgccctctgcagccctctgccagcctcttgg | |
| | | gggcagttccggcctctccgacttccccactggccacactccattcagactcctttcctgccttg | |
| | | tgacctcagatggtcaccatcattcctgtgctcagaggccaacccatcacaggggtgagatag | |
| | | gttggggcctgccctaacccgccagcctcctcctctcgggctggatctgggggctagcagtg | |
| | | agtacccgcatggtatcagcctgcctctcccgcccacgccctgctgtctccaggcctatagac | |
| | | gtttctctccaaggccctatcccccaatgttgtcagcagatgcctggacagcacagccacccat | |
| | | ctcccattcacatggcccacctcctgcttcccagaggactggccctacgtgctctctctcgtcct | |
| | | acctatcaatgcccagcatggcagaacctgcagcccttggccactgcagatggaaacctctc | |
| | | agtgtcttgacatcaccctacccaggcggtgggtctccaccacagccactttgagtctgtggtc | |
| | | cctggagggtggcttctcctgactggcaggatgaccttagccaagatattcctctgttccctctg | |
| | | ctgagataaagaattcccttaacatgatataatccacccatgcaaatagctactggcccagcta | |
| | | ccatttaccatttgcctacagaatttcattcagtctacactttggcattctctctggcgatggagtgt | |
| | | ggctgggctgaccgcaaaaggtgccttacacactgcccccaccctcagccgttgccccatca | |
| | | gaggctgcctcctccttctgattaccccccatgttgcatatcagggtgctcaaggattggagag | |
| | | gagacaaaaccaggagcagcacagtggggacatctcccgtctcaacagccccaggcctatg | |
| | | ggggctctggaaggatgggccagcttgcaggggttggggagggagacatccagcttgggc | |
| | | tttcccctttggaataaaccattggtctgtcaaaaaaaaaaaaaaaaaaa | |
| UNC45A | NM_001039675.1 | acttaacaaccgaagtaacccgcaatgcggaagggcgaggggattgcgagtcaccgagttt | 36 |
| | | cccgcgcggcttgagtcacggcctagaaagagagatgttggggttcccaggaccaggaca | |
| | | gaggtggtagtgaactctcatgggcatccagagaaggtcaggccccttgctgacaggcctat | |
| | | ctgtggggctactgctgctcttcagctgggtgacccttgtccagccaacctctctctcagctctg | |
| | | gtccaccaccctcacttgtgccagaccacccgggatgtccatggccgtcactaccctggtttct | |
| | | tttgccctcgtctgtctgattctccagaggaagcctactgctgccacctgcaggctgcagggg | |
| | | gctcctgctgcacccgggctgaatttgaggccctgtaccaagtcaatctgtccgctcttccgcc | |
| | | cccgcccatcctcaggggcccaggcccgctcctagtgctgggcctctacaacctactggttgt | |
| | | gaccctgatgaccgtagacctcgtgcacttctgctgcggtcggggccggagtctgggctgga | |
| | | gccaccgcaggcctccctctgggtcctccgccgcgagctccctgcaggtctctgcggggac | |
| | | agcttaggtgcgcccggagcttgcctgcacctgcgatccagagccaagcgccccgcccctg | |
| | | cccgggcgcgctccctccttagccctgcccctctctgaccccacctccgacgcaagagtggg | |
| | | gcggggcagctgccggtggcgtcccgaacccagactcgccccgccccagagactgcgcct | |
| | | gcgcgggcacgagacaacctctccgcgatgactgccagctcagtggagcagctgcggaag | |
| | | gagggcaatgagctgttcaaatgtggagactacgggggcgccctggcggcctacactcagg | |
| | | ccctgggtctggacgcgacgccccaggaccaggccgttctgcaccggaaccgggccgcct | |
| | | gccacctcaagctggaagattacgacaaagcagaaacagaggcatccaaagccattgaaaa | |
| | | ggatggtggggatgtcaaagcactctaccggcggagccaagccctagagaagctgggccg | |
| | | cctggaccaggctgtccttgacctgcagagatgtgtgagcttggagcccaagaacaaagtttt | |
| | | ccaggaggccttgcggaacatcgggggccagattcaggagaaggtgcgatacatgtcctcg | |
| | | acggatgccaaagtggaacagatgtttcagatactgttggacccagaagagaagggcactga | |
| | | gaaaaagcaaaaggcttctcagaacctggtggtgctggccagggaggatgctggagcgga | |
| | | gaagatcttccggagtaatggggttcagctcttgcaacgtttactggacatgggagagactga | |
| | | cctcatgctggcggctctgcgtacgctggttggcatttgctctgagcatcagtcacggacagtg | |
| | | gcaaccctgagcatactgggaactcggcgagtagtctccatcctgggcgtggaaagccagg | |
| | | ctgtgtccctggctgcctgccacctgctgcaggttatgtttgatgccctcaaggaaggtgtcaa | |
| | | aaaaggcttccgaggcaaagaaggtgccatcattgtggatcctgcccgggagctgaaggtc | |
| | | ctcatcagtaacctcttagatctgctgacagaggtgggggtctctggccaaggccgagacaat | |
| | | gccctgaccctcctgattaaagcggtgccccggaagtctctcaaggaccccaacaacagcct | |
| | | caccctctgggtcatcgaccaaggtctgaaaaagattttggaagtggggggctctctacagga | |
| | | ccctcctggggagctcgcagtgaccgcaaacagccgcatgagcgcctctattctcctcagca | |
| | | agctctttgatgacctcaagtgtgatgcggagagggagaatttccacagactttgtgaaaacta | |
| | | catcaagagctggtttgagggccaagggctggccgggaagctacgggccatccagacggt | |
| | | gtcctgcctcctgcagggcccatgtgacgctggcaaccgggccttggagctgagcggtgtca | |
| | | tggagagtgtgattgctctgtgtgcctctgagcaggaggaggagcagctggtggccgtggag | |
| | | gctctgatccatgcagccggcaaggctaagcgggcctcattcatcactgccaatggtgtctcg | |
| | | ctgctgaaggacctatataagtgcagcgagaaggacagcatccgcatccgggcgctagtgg | |
| | | gactctgtaagctcggttcggctggagggactgacttcagcatgaagcagtttgctgaaggct | |
| | | ccactctcaaactggctaagcagtgtcgaaagtggctgtgcaatgaccagatcgacgcaggc | |
| | | actcggcgctgggcagtggagggcctggcttacctgacctttgatgccgacgtgaaggaag | |
| | | agtttgtggaggatgcggctgctctgaaagctctgttccagctcagcaggttggaggagaggt | |
| | | cagtgctctttgcggtggcctcagcgctggtgaactgcaccaacagctatgactacgaggag | |
| | | cccgaccccaagatggtggagctggccaagtatgccaagcagcatgtgcccgagcagcac | |
| | | cccaaggacaagccaagcttcgtgcgggctcgggtgaagaagctgctggcagcgggtgtg | |
| | | gtgtcggccatggtgtgcatggtgaagacggagagccctgtgctgaccagttcctgcagaga | |
| | | gctgctctccagggtcttcttggctttagtggaagaggtagaggaccgaggcactgtggttgc | |
| | | ccagggaggcggcagggcgctgatcccgctggccctggaaggcacggacgtggggcag | |
| | | acaaaggcagcccaggcccttgccaagctcaccatcacctccaacccggagatgaccttccc | |
| | | tggcgagcggatctatgaggtggtccggcccctcgtctccctgttgcacctcaactgctcagg | |
| | | cctgcagaacttcgaggcgctcatggccctaacaaacctggctgggatcagcgagaggctc | |
| | | cggcagaagatcctgaaggagaaggctgtgcccatgatagaaggctacatgtttgaggagc | |
| | | atgagatgatccgccgggcagccacggagtgcatgtgtaacttggccatgagcaaggaggt | |
| | | gcaggacctcttcgaagcccagggcaatgaccgactgaagctgctggtgctgtacagtgga | |
| | | gaggatgatgagctgctacagcgggcagctgccgggggcttggccatgcttacctccatgc | |
| | | ggcccacgctctgcagccgcattccccaagtgaccacacactggctggagatcctgcaggc | |
| | | cctgcttctgagctccaaccaggagctgcagcaccggggtgctgtggtggtgctgaacatgg | |
| | | tggaggcctcgagggagattgccagcaccctgatggagagtgagatgatggagatcttgtca | |
| | | gtgctagctaagggtgaccacagccctgtcacaagggctgctgcagcctgcctggacaaag | |
| | | cagtggaatatgggcttatccaacccaaccaagatggagagtgagggggttgtccctgggcc | |
| | | caaggctcatgcacacgctacctattgtggcacggagagtaaggacggaagcagctttggct | |
| | | ggtggtggctggcatgcccaatactcttgcccatcctcgcttgctgccctaggatgtcctctgtt | |
| | | ctgagtcagcggccacgttcagtcacacagccctgcttggccagcactgcctgcagcctcact | |
| | | cagaggggccctttttctgtactactgtagtcagctgggaatggggaaggtgcatcccaacac | |
| | | agcctgtggatcctggggcatctggaagggcgcacacatcagcagcctcaccagctgtgag | |
| | | cctgctatcaggcctgcccctccaataaaagtgtgtagaactccaaaaaaaaaaaaaaaaaaa | |
| | | aaaaaaaaaaaaaaa | |
| XPC | NM_004628.4 | cgaaggggcgtggccaagcgcaccgcctcggggcggggccggcgttctagcgcatcgcg | 37 |
| | | gccgggtgcgtcactcgcgaagtggaatttgcccagacaagcaacatggctcggaaacgcg | |
| | | cggccggcggggagccgcggggacgcgaactgcgcagccagaaatccaaggccaagag | |
| | | caaggcccggcgtgaggaggaggaggaggatgcctttgaagatgagaaacccccaaaga | |
| | | agagccttctctccaaagtttcacaaggaaagaggaaaagaggctgcagtcatcctgggggt | |
| | | tcagcagatggtccagcaaaaaagaaagtggccaaggtgactgttaaatctgaaaacctcaa | |
| | | ggttataaaggatgaagccctcagcgatggggatgacctcagggactttccaagtgacctca | |
| | | agaaggcacaccatctgaagagaggggctaccatgaatgaagacagcaatgaagaagagg | |
| | | aagaaagtgaaaatgattgggaagaggttgaagaacttagtgagcctgtgctgggtgacgtg | |
| | | agagaaagtacagccttctctcgatctcttctgcctgtgaagccagtggagatagagattgaaa | |
| | | cgccagagcaggcgaagacaagagaaagaagtgaaaagataaaactggagtttgagacat | |
| | | atcttcggagggcgatgaaacgtttcaataaaggggtccatgaggacacacacaaggttcac | |
| | | cttctctgcctgctagcaaatggcttctatcgaaataacatctgcagccagccagatctgcatgc | |
| | | tattggcctgtccatcatcccagcccgctttaccagagtgctgcctcgagatgtggacacctac | |
| | | tacctctcaaacctggtgaagtggttcattggaacatttacagttaatgcagaactttcagccagt | |
| | | gaacaagataacctgcagactacattggaaaggagatttgctatttactctgctcgagatgatg | |
| | | aggaattggtccatatattcttactgattctccgggctctgcagctcttgacccggctggtattgt | |
| | | ctctacagccaattcctctgaagtcagcaacagcaaagggaaagaaaccttccaaggaaaga | |
| | | ttgactgcggatccaggaggctcctcagaaacttccagccaagttctagaaaaccacaccaaa | |
| | | ccaaagaccagcaaaggaaccaaacaagaggaaacctttgctaagggcacctgcaggcca | |
| | | agtgccaaagggaagaggaacaagggaggcagaaagaaacggagcaagccctcctccag | |
| | | cgaggaagatgagggcccaggagacaagcaggagaaggcaacccagcgacgtccgcat | |
| | | ggccgggagcggcgggtggcctccagggtgtcttataaagaggagagtgggagtgatgag | |
| | | gctggcagcggctctgattttgagctctccagtggagaagcctctgatccctctgatgaggatt | |
| | | ccgaacctggccctccaaagcagaggaaagcccccgctcctcagaggacaaaggctgggt | |
| | | ccaagagtgcctccaggacccatcgtgggagccatcgtaaggacccaagcttgccagcggc | |
| | | atcctcaagctcttcaagcagtaaaagaggcaagaaaatgtgcagcgatggtgagaaggca | |
| | | gaaaaaagaagcatagctggtatagaccagtggctagaggtgttctgtgagcaggaggaaa | |
| | | agtgggtatgtgtagactgtgtgcacggtgtggtgggccagcctctgacctgttacaagtacg | |
| | | ccaccaagcccatgacctatgtggtgggcattgacagtgacggctgggtccgagatgtcaca | |
| | | cagaggtacgacccagtctggatgacagtgacccgcaagtgccgggttgatgctgagtggt | |
| | | gggccgagaccttgagaccataccagagcccatttatggacagggagaagaaagaagactt | |
| | | ggagtttcaggcaaaacacatggaccagcctttgcccactgccattggcttatataagaaccac | |
| | | cctctgtatgccctgaagcggcatctcctgaaatatgaggccatctatcccgagacagctgcc | |
| | | atccttgggtattgtcgtggagaagcggtctactccagggattgtgtgcacactctgcattccag | |
| | | ggacacgtggctgaagaaagcaagagtggtgaggcttggagaagtaccctacaagatggtg | |
| | | aaaggcttttctaaccgtgctcggaaagcccgacttgctgagccccagctgcgggaagaaaa | |
| | | tgacctgggcctgtttggctactggcagacagaggagtatcagcccccagtggccgtggacg | |
| | | ggaaggtgccccggaacgagtttgggaatgtgtacctcttcctgcccagcatgatgcctattg | |
| | | gctgtgtccagctgaacctgcccaatctacaccgcgtggcccgcaagctggacatcgactgt | |
| | | gtccaggccatcactggctttgatttccatggcggctactcccatcccgtgactgatggatacat | |
| | | cgtctgcgaggaattcaaagacgtgctcctgactgcctgggaaaatgagcaggcagtcattg | |
| | | aaaggaaggagaaggagaaaaaggagaagcgggctctagggaactggaagttgctggcc | |
| | | aaaggtctgctcatcagggagaggctgaagcgtcgctacgggcccaagagtgaggcagca | |
| | | gctccccacacagatgcaggaggtggactctcttctgatgaagaggaggggaccagctctca | |
| | | agcagaagcggccaggatactggctgcctcctggcctcaaaaccgagaagatgaagaaaa | |
| | | gcagaagctgaagggtgggcccaagaagaccaaaagggaaaagaaagcagcagcttccc | |
| | | acctgttcccatttgagcagctgtgagctgagcgcccactagaggggcacccaccagttgct | |
| | | gctgccccactacaggccccacacctgccctgggcatgcccagcccctggtggtgggggct | |
| | | tctctgctgagaaggcaaactgaggcagcatgcacggaggcggggtcaggggagacgag | |
| | | gccaagctgaggaggtgctgcaggtcccgtctggctccagcccttgtcagattcacccaggg | |
| | | tgaagccttcaaagctttttgctaccaaagcccactcaccctttgagctacagaacactttgcta | |
| | | ggagatactcttctgcctcctagacctgttctttccatctttagaaacatcagtttttgtatggaagc | |
| | | caccgggagatttctggatggtggtgcatccgtgaatgcgctgatcgtttcttccagttagagtc | |
| | | ttcatctgtccgacaagttcactcgcctcggttgcggacctaggaccatttctctgcaggccact | |
| | | taccttcccctgagtcaggcttactaatgctgccctcactgcctctttgcagtaggggagagag | |
| | | cagagaagtacaggtcatctgctgggatctagttttccaagtaacattttgtggtgacagaagc | |
| | | ctaaaaaaagctaaaatcaggaaagaaaaggaaaaatacgaattgaaaattaaggaaatgtta | |
| | | gtaaaatagatgagtgttaaactagattgtattcattactagataaaatgtataaagctctctgtac | |
| | | taaggagaaatgacttttataacattttgagaaaataataaagcatttatctaaaaaaaaaaa | |
| ALG9 | NM_001077690.1 | gtcttttgtccctcggcggacaccgtttgccagccaaagctatgtctgcgcgctcaccgacttc | 38 |
| | | atagggtgccgaattcttttttccccaggcttgccatggctagtcgaggggctcggcagcgcct | |
| | | gaagggcagcggggccagcagtggggatacggccccggctgcggacaagctgcgggag | |
| | | ctgctgggcagccgagaggcgggcggcgcggagcaccggaccgagttatctgggaacaa | |
| | | agcaggacaagtctgggcacctgaaggatctactgctttcaagtgtctgctttcagcaaggtta | |
| | | tgtgctgctctcctgagcaacatctctgactgtgatgaaacattcaactactgggagccaacac | |
| | | actacctcatctatggggaagggtttcagacttgggaatattccccagcatatgccattcgctcc | |
| | | tatgcttacctgttgcttcatgcctggccagctgcatttcatgcaagaattctacaaactaataag | |
| | | attcttgtgttttactttttgcgatgtcttctggcttttgtgagctgtatttgtgaactttacttttacaag | |
| | | gctgtgtgcaagaagtttgggttgcacgtgagtcgaatgatgctagccttcttggttctcagcac | |
| | | tggcatgttttgctcatcatcagcattccttcctagtagcttctgtatgtacactacgttgatagcca | |
| | | tgactggatggtatatggacaagacttccattgctgtgctgggagtagcagctggggctatctt | |
| | | aggctggccattcagtgcagctcttggtttacccattgcctttgatttgctggtcatgaaacacag | |
| | | gtggaagagtttctttcattggtcgctgatggccctcatactatttctggtgcctgtggtggtcatt | |
| | | gacagctactattatgggaagttggtgattgcaccactcaacattgttttgtataatgtctttactcc | |
| | | tcatggacctgatctttatggtacagaaccctggtatttctatttaattaatggatttctgaatttcaa | |
| | | tgtagcctttgctttggctctcctagtcctaccactgacttctcttatggaatacctgctgcagaga | |
| | | tttcatgttcagaatttaggccacccgtattggcttaccttggctccaatgtatatttggtttataattt | |
| | | tcttcatccagcctcacaaagaggagagatttcttttccctgtgtatccacttatatgtctctgtgg | |
| | | cgctgtggctctctctgcacttcagaaatgttaccactttgtgtttcaacgatatcgcctggagca | |
| | | ctatactgtgacatcgaattggctggcattaggaactgtcttcctgtttgggctcttgtcattttctc | |
| | | gctctgtggcactgttcagaggatatcacgggccccttgatttgtatccagaattttaccgaattg | |
| | | ctacagacccaaccatccacactgtcccagaaggcagacctgtgaatgtctgtgtgggaaaa | |
| | | gagtggtatcgatttcccagcagcttccttcttcctgacaattggcagcttcagttcattccatca | |
| | | gagttcagaggtcagttaccaaaaccttttgcagaaggacctctggccacccggattgttccta | |
| | | ctgacatgaatgaccagaatctagaagagccatccagatatattgatatcagtaaatgccattat | |
| | | ttagtggatttggacaccatgagagaaacaccccgggagccaaaatattcatccaataaagaa | |
| | | gaatggatcagcttggcctatagaccattccttgatgcttctagatcttcaaagctgctgcgggc | |
| | | attctatgtccccttcctgtcagatcagtatacagtgtacgtaaactacaccatcctcaaaccccg | |
| | | gaaagcaaagcaaatcaggaagaaaagtggaggttagcaacacacctgtggccccaaagg | |
| | | acaaccatcttgttaactattgattccagtgacctgactccctgcaagtcatcgcctgtaacatttg | |
| | | taataaaggtcttctgacatgaatactggaatctgggtgctctgggctagtcaaagtctatttcaa | |
| | | agtctaatcaaagtcacatttgctccctgtgtgtgtctctgttctgcatgtaaactttttgcagctag | |
| | | gcagagaaaggccctaaagcacagatagatatattgctccacatctcattgtttttcctctgttca | |
| | | attatttactagaccggagaagagcagaaccaacttacaggaagaattgaaaatcctggtact | |
| | | ggatggctgtgataagctgttctccacactctggcctggcatctgagaactagcaagcctctctt | |
| | | aggccatatgggcttctccaccaaagctgtttggcagctcctagcagaccttcttattgaaatcc | |
| | | tcatgctgaaaatgaacacagcctagttgccaacccacatgtccttttcacctccagcaagact | |
| | | aagcttctttaaagcacttcacaggactaggaccctgtcctggagctatctcaggaaaaaggtg | |
| | | accatttgaggaactgtgacctaattttattataatgatgcctctaattttcatttcctttacaaccaa | |
| | | ctgtaactataaggttgtattgcttttttgttcagttttagcatgctattttttgaattctagactcctcc | |
| | | atgtgaagatatcaacagacaaaactacaactgtataggacatatttggagaaaattctatcaat | |
| | | tgatacatttggatgacatcacatttttaagtaatgtaatctgaggccattgctgaggaaattaag | |
| | | aattttcctttttttttaaccacccccagtgaaaaggatcagtgtatatttatagcacctattttttagt | |
| | | tctgtctgttgtgaggcacatcctgcatggggcacttctagtcaaataggcaatgataaggacc | |
| | | taattaaaatgtgataagtgtatactattactttaaaagcctttacagtcagtacttcagtttacaag | |
| | | gcactttcacagcatctcgtttgatcctcacagtcacaacatgtggtagacaaggcaggtgattt | |
| | | ttatccccattttacagataaggaaacaggctgcgggtggggagtgaggggaggtaaagata | |
| | | gttagttgcctaaggtcacacagccagtaagtaatagagctgggactggaacccaggtttcctt | |
| | | actctcatctattgctcctccatattcctcactcaaccatgaaaacattacttgaaaggactgatga | |
| | | ggttaaccagagacctaactgatattgtaactttctattttaaggaagaattgtgtctgtatttgagt | |
| | | tctttggagcctccagtctgcctgtgtgttagaccagcacagcagtgctgtgtgatgcagcctg | |
| | | acctgtggcaggaaagtagtgcttctgtttggaagtcatgttcttttgcagccacacaggatcca | |
| | | aatatcagtactattcctgtagtcaatctggggtcacattataggtgccttatttccctaagggtaa | |
| | | ctgatctgaatatctgcaaataggatgaatctatttttcagaagttccatctttcatttttcttttttttttt | |
| | | gagacagagtctcattctgtcgcccatgctggagtgcagtggcgcgatctcggctcgctgcaa | |
| | | cctctgcctcccaggttgaagcaattctcatgcctcagccacccgagtagctgggattacagg | |
| | | catgcgccatcatgcccagctaatttatgtatttttagtagagttggagtttcaccatgttggccag | |
| | | gctggtcttggactcctgacctcaggtcatccacccgcctcagcctcccaaagtgctggtatta | |
| | | caggcgtgagccaccgcacccagccccatctttcattttcaaagagaagggcattctaatagg | |
| | | aactggtgccaagagagaagaaaagaagtgataacagaagaaatggctagttacaatattaa | |
| | | aaagctcctctttgagatctcctctgcaggaatatcagagacggagttgaagcgctggagagg | |
| | | taataggtctagacagtacagaacaataactggggagtgtgtgaggatagactgggctcccc | |
| | | cttgcttgaaagatctctggcatttaattctcaattcttgattactattttccagtgtaaaactagcac | |
| | | atatgatctgactacaggacagagaattttaagtgaaacatttgccttacttgcagtaataatgtg | |
| | | ctgttcttcacagtagctaaggccctctatgtttcccagaggtaaataagaatccaggaatgga | |
| | | ggtccatctgtgatgaatggcttttttctaatcaaagtagtataatgctgttttatctgttttgtcatctt | |
| | | gtttttttttttttttaaaaaaacaaaaccttaattataatatagcgcaaagaaaggccaggactgat | |
| | | gcagggattccttggaaatatcagttcctatcacttttaaaacctgattttggatctctctgttctat | |
| | | gtatgtctttagtgagagcacaatacatggcagaacgctgtgccaaatgttataggtaaggaat | |
| | | atagaaatgaatgttttttgttgtgaaggtgttttcatgtgatattttataaacacattttaaaaaatct | |
| | | ccatcactttttagtataggaaggatagctttgcctgggaaaaacagtttcaacacacctgctca | |
| | | gagtagcagttctccctcaaaaaagcagtgttcagcctgcactgactgttctgcttgccaaaag | |
| | | gaggaagcatgcaagatacttatttctccatagattgtggagtatagagggatgtgggactaca | |
| | | gattattattttttttccccgagacagagtcttgctctgtcgcccaggttggaacacaatggcacg | |
| | | acctcagctcactgcaacctctgtctcccgggttcaagcaattctcctgcttcagcctcctgagt | |
| | | agctgggattacaggcacacaccaccaccgcactcagctaatttttgtatttttagtagaggtgg | |
| | | ggttttaccatgttggccaggctggtcttaaactcctgaccttgtaatcatcccgcctcggcctcc | |
| | | taaagtgctaggattacaggcatgagccaccgcacccggcccagataatttttaatagcctttg | |
| | | atcatggggtgagtgagggagtaggtatacttggcaaatgcatggttctctgatttctagctcta | |
| | | aagcagccttatctgaatccccaaatcttgtgatgctgagtaccattactgaaccagtctgcacg | |
| | | gtaggcatctgctaccaaaatttacctcctacctggtaggtgtcatctgataagaaagaagaca | |
| | | ggttattttaattttttgagataatcacagaaaattgcagcccatactctttattaccgaattcaagtt | |
| | | tggaaatagaccctttgttttaaatcatgatgggtctttatcccaatcatttatctgggtcatttttcc | |
| | | aactttggagttctaggaaagaaccttgaaaacctgatatgattctgcagcatgaggtctacggt | |
| | | gaccatttgggcaaagctccagtggcaatcatttattgtgttttgcatttcctgggatttattgaaat | |
| | | aagaattcactgtgattatgtagtcttctggctagtatcaggcagctctgcttttaatttggttaattt | |
| | | tattttctctgaagagggagaagaggtacaatttaatcttggcctccacaagcatattaaagctc | |
| | | acgtgttaatcagtgcattcttatgctcctacattaaatgccttgggtaaatggataaatggacat | |
| | | gtgcccagctttaattttttttgcaacagaaagatcagacttccgtatggcatcgttggatttcaga | |
| | | ggctttctggtgtatctgtaaatctgaatgttgccttctgccagtctgtataaccaggtgattcatg | |
| | | ctgcaaatgaaatcaggaagcagtaaagtgttaaagcaagagtattgtccaattcacttgtcttc | |
| | | ctgatccttgtactttatttcacgtgtcggtgtttacattacatacttatatttcctgtgaaagaaaga | |
| | | gttaaataaattgtagcagtttga | |
| SEPN | NM_031475.2 | agcggagcgccaggcagcgcggagcggaggccaggcccacagccgctccgcctcccgg | 39 |
| | | cccgcagatccccgacggccgcaccgcgggctcctctggcccgcaagaacacgtgcatgg | |
| | | cgtcctggggaaggcgctgagtgcggagtcgcggcgccgcacgcggcaccatggccctg | |
| | | gagcaggcgctgcaggcggcgcggcagggcgagctggacgtgctgaggtcgctgcacgc | |
| | | cgcaggcctcctggggccctcgctgcgcgacccgctggacgcgctgcccgtgcaccacgc | |
| | | ggcccgcgctgggaagctgcactgtctgcgcttcctggtggaggaagccgccctccccgcc | |
| | | gcggcccgcgcccgcaacggcgccacaccggcccacgacgcctccgccaccggccacct | |
| | | cgcctgcctgcagtggctgctgtcgcagggcggctgcagagtgcaggacaaagacaattct | |
| | | ggtgccacagtcttgcatctggctgcccgcttcggccaccccgaggtggtgaactggctcttg | |
| | | catcatggcggtggggaccccaccgcggccacagacatgggcgccctgcctatccactacg | |
| | | ctgccgccaaaggagacttcccctccctgaggcttctcgtcgagcactaccctgagggagtg | |
| | | aatgcccaaaccaagaacggtgccacgcccctgtacctggcgtgccaggagggccacctg | |
| | | gaggtgacccagtacctggtgcaggaatgcggcgcagacccgcacgcgcgcgcccacga | |
| | | cggcatgaccccgctgcacgccgcggcgcagatgggccacagcccagtcatcgtgtggttg | |
| | | gtgagctgcaccgacgtgagcctgtccgagcaggacaaagacggcgccaccgccatgcac | |
| | | ttcgcggcgagccgcggccacaccaaggtgctcagctggctgctgctgcacggcggggag | |
| | | atctcggctgacctgtggggcgggaccccgctgcacgacgccgccgagaacggggagcta | |
| | | gagtgctgccagatcctggtagtgaacggcgcggagctggacgtccgcgaccgcgacggg | |
| | | tacacggccgccgacctgtcggacttcaacggccacagccactgcacccgctacctgcgca | |
| | | cggtggagaacctgagcgtggagcaccgcgtgctttcccgggatccatccgcagagctgga | |
| | | ggctaagcagccggattcaggcatgtcctcacccaataccacggtgtcggtccagccgctga | |
| | | actttgacctcagctcgcctaccagcaccctctccaactacgactcctgctcctccagccactc | |
| | | cagcatcaagggccagcaccctccatgtgggctttccagcgctagagctgcagacatacaga | |
| | | gctacatggacatgctgaacccggagctgggcctgcctcggggcacgattgggaagcccac | |
| | | acccccaccacccccacccagcttccccccgccacccccgcccccaggcacccaactgcc | |
| | | cccacccccacctggctacccagctcccaagcctcctgtaggaccacaggcagctgacatct | |
| | | acatgcagaccaagaacaaactccgccacgtggagacagaggccctcaagaaggagctga | |
| | | gctcctgtgacggccacgacgggctgcggaggcaggactccagccgcaagccccgcgcc | |
| | | ttcagcaagcagcccagcacgggggactactaccggcagctgggccgctgccccggcga | |
| | | gacgctggccgcacgcccgggcatggcgcacagcgaggaggtgcgtgcccgccagccc | |
| | | gcgcgcgccggctgcccgcgcctcggccctgccgcccgcggctcactcgaaggcccctcc | |
| | | gctcccccgcaggcggcgctgcttcctgggaaccatgttcctaacggctgcgccgcggacc | |
| | | ccaaggcgtccagggagctgccaccgccgcccccaccgccgccgccgcccctgccggag | |
| | | gccgcgagttcgccaccgccggccccgcctctgcccctcgagagcgctggccctggctgc | |
| | | gggcagcgccgctcctcctcgtccaccggcagcaccaagtctttcaacatgatgtccccgac | |
| | | gggcgacaactcggagctactggctgagattaaggcaggcaagagcctgaagccgacgcc | |
| | | ccagagcaaggggctgaccacagtgttctcaggcatcgggcagccggccttccagcccgat | |
| | | tcgccgctgccttctgtgtcacctgcactgtcaccagtccggagccccacaccgccagctgcg | |
| | | gggtttcagccgctgctcaatggaagcttggttcccgtgccgcccactactcctgcgccggga | |
| | | gtgcagctggacgtggaggctctcatccccacgcacgatgagcagggccggcccatcccc | |
| | | gagtggaagcgccaggtgatggtgcgcaagatgcagctgaagatgcaggaggaggagga | |
| | | gcagaggcggaaggaggaggaggaggaggcccggctggccagcatgcccgcctggag | |
| | | gcgggacctcctgcggaagaagctggaagaagagagggagcagaagcggaaagaggag | |
| | | gagcgacagaagcaggaggagctgcggcgggagaaggaacagtcagagaagctgcgga | |
| | | cgctgggctacgatgagagcaagctggcgccctggcagcgacaggtcatcctgaagaagg | |
| | | gggacatcgctaagtactagaggccgcagactcctgtccgcagcctcgcagctccgtgggg | |
| | | ccctccgccccagccccagccagccaggccctggtggaaaggctgggagccgcacagcc | |
| | | ctcccctcctgcgctggaaaccctccctgacccccaccctggccccccgtatccccagccctt | |
| | | ggcaacactggagtgcacacgccgccacggttgcccagaaaaagtgcccaagctgctgac | |
| | | gcaaacaacaacaaatgctgcttatttgcatgccgacttacatatatttgcatgttcgttgactatc | |
| | | aaagagtgcagagctctccccagccccgtgggtggtgactttgttttcctgcggggctcagcc | |
| | | ccctccaggatgcagccccctcccccgcaccccggaaccggcgtcgctggcgcatcctgg | |
| | | gtggaggcaggccccgagctcggggaaggggttttcccttcctctctgacccagatctgcgc | |
| | | gcggcctagcccgggcctcatttcttatccccgccaagggtttcctctcagtcatttgtttaccag | |
| | | aaacatgaaaactgcctgtctggccgggccgcacttgtggcccccgggaccccacctctggc | |
| | | cccacctccctcaagtctgcgccccgtccccagccagacccactcgctgccgggaccctttc | |
| | | actgccccggtggagtgaatagaggatgaggggccctgaccctgtgtctccaactgctgcac | |
| | | cccatcccgaccctgtctccgccacctcgcagccccattaaagcgctctcatctgggctccgg | |
| | | ttcactca | |
| YWHAQ | NM_006826.3 | ttgggcggtggaccgcccctcggccccggggtaggctgacacgggagggtcctcagctaa | 40 |
| | | agccaaaagcagatcaaagtggtgggactcgcgtcgcggccgcggagacgtgaagctctc | |
| | | gaggctcctcccgctgcgggtcggcgctcgccctcgctctcctcgccctccgccccggcccc | |
| | | ggccccgcgcccgccatggagaagactgagctgatccagaaggccaagctggccgagca | |
| | | ggccgagcgctacgacgacatggccacctgcatgaaggcagtgaccgagcagggcgccg | |
| | | agctgtccaacgaggagcgcaacctgctctccgtggcctacaagaacgtggtcgggggccg | |
| | | caggtccgcctggagggtcatctctagcatcgagcagaagaccgacacctccgacaagaag | |
| | | ttgcagctgattaaggactatcgggagaaagtggagtccgagctgagatccatctgcaccac | |
| | | ggtgctggaattgttggataaatatttaatagccaatgcaactaatccagagagtaaggtcttct | |
| | | atctgaaaatgaagggtgattacttccggtaccttgctgaagttgcgtgtggtgatgatcgaaaa | |
| | | caaacgatagataattcccaaggagcttaccaagaggcatttgatataagcaagaaagagatg | |
| | | caacccacacacccaatccgcctggggcttgctcttaacttttctgtattttactatgagattctta | |
| | | ataacccagagcttgcctgcacgctggctaaaacggcttttgatgaggccattgctgaacttga | |
| | | tacactgaatgaagactcatacaaagacagcaccctcatcatgcagttgcttagagacaaccta | |
| | | acactttggacatcagacagtgcaggagaagaatgtgatgcggcagaaggggctgaaaact | |
| | | aaatccatacagggtgtcatccttctttccttcaagaaacctttttacacatctccattccttattcca | |
| | | cttggatttcctatagcaaagaaacccattcatgtgtatggaatcaactgtttatagtcttttcacac | |
| | | tgcagctttgggaaaacttcattccttgatttgtgtttgtcttggccttcctggtgtgcagtactgct | |
| | | gtagaaaagtattaatagcttcatttcatataaacataagtaactcccaaacacttatgtagagga | |
| | | ctaaaaatgtatctggtatttaagtaatctgaaccagttctgcaagtgactgtgttttgtattactgt | |
| | | gaaaataagaaaatgtagttaattacaatttaaagagtattccacataacttcttaatttctacattc | |
| | | cctcccttactcttcgggggtttcctttcagtaagcaacttttccatgctcttaatgtattcctttttag | |
| | | taggaatccggaagtattagattgaatggaaaagcacttgccatctctgtctaggggtcacaaa | |
| | | ttgaaatggctcctgtatcacatacggaggtcttgtgtatctgtggcaacagggagtttccttatt | |
| | | cactctttatttgctgctgtttaagttgccaacctcccctcccaataaaaattcacttacacctcctg | |
| | | cctttgtagttctggtattcactttactatgtgatagaagtagcatgttgctgccagaatacaagca | |
| | | ttgcttttggcaaattaaagtgcatgtcatttcttaatacactagaaaggggaaataaattaaagta | |
| | | cacaagtccaagtctaaaactttagtacttttccatgcagatttgtgcacatgtgagagggtgtcc | |
| | | agtttgtctagtgattgttatttagagagttggaccactattgtgtgttgctaatcattgactgtagtc | |
| | | ccaaaaaagccttgtgaaaatgttatgccctatgtaacagcagagtaacataaaataaaagtac | |
| | | attttataaaccatttactatggctttgtaacaattgcatacccatattttaagggacaggtgaattt | |
| | | actactttctaaagtttattgatacttcccttttatgtaaaatgtagtagtgatacctatatttccacatt | |
| | | gtgcattgtgacacacttgtctagggatgcctggaagtgtataaaattggactgcatttcttaga | |
| | | gtgttttactatagatcagtctcatgggccatctcttcctcagatgtaaatgatatctggttaagtgt | |
| | | tatatggaataaagtggacattttaaaactagcaaagttaaaaaaaaaaaaaaaaaaaa | |
| VPS37A | NM_001145152.1 | gcagagggggcggagagcgcccccgggggcggggcacgcaagtgacggcggcgcgg | 41 |
| | | gtggtggagcgctgggcggccaggctccctggctggccggtttgggcgtctgggccgtgaa | |
| | | ggtgggacctcctgttccgggccgcaagtttccctctccagccgcccgccgttcgtagcatgt | |
| | | cccccagaactcggggagcgcaggcaggacaggcttagagaagacgcggtccccagcgc | |
| | | ttgggccacggacgtcccaccccgctcctctgtcgctggagaaccgccgggccgagccact | |
| | | gggagaagcaggccagagccttccagggcctccggcccgtggacccgaggaggatgagc | |
| | | tggctttttcccctgaccaagagcgcctcctcctccgcggctgggtcccccggtggcctcacc | |
| | | agcctccagcagcagaagcagcgcctgatcgagtccctccggaactcacactccagattgct | |
| | | tcctccacagtttcctcaggaaaaaccagtgatcagtgtttatccaccaatacgacatcacttaat | |
| | | ggataaacaaggagtgtatgttacctctccattagtaaacaattttacaatgcactcagatcttgg | |
| | | aaaaattattcagagtctgttggatgagttttggaagaatcctccagttttagctcctacttcaaca | |
| | | gcatttccttatctatacagtaacccaagtgggatgtctccttatgcttctcagggttttccatttctt | |
| | | cctccatatcctccacaagaagcaaacaggagtatcacttctttatctgttgctgacactgtttctt | |
| | | cttcaacaacaagtcataccacagccaagcctgccgctccttcatttggtgtcctttcaaatctgc | |
| | | cattacccattcccacagtggatgcttcaataccgacaagccaaaatggttttgggtacaagat | |
| | | gccagatgtccctgatgcatttccagaactctcagaactaagtgtgtcacaactcacagatatg | |
| | | aatgaacaagaggaggtattactagaacagtttctgactttgcctcaactaaaacaaattattac | |
| | | cgacaaagatgacttagtaaaaagtattgaggaactagcaagaaaaaatctccttttggagccc | |
| | | agcttggaagccaaaagacaaactgttttagataagtatgaattacttacacagatgaagtcca | |
| | | ctttcgaaaagaagatgcaaaggcagcatgaacttagtgagagctgtagtgcaagtgcccttc | |
| | | aggcaagattgaaagtagctgcacatgaagctgaggaagaatctgataatattgcagaagact | |
| | | tcttggagggaaagatggaaatagatgattttctcagtagcttcatggaaaagagaacaatttg | |
| | | ccactgtagaagagccaaggaagagaaacttcagcaggcgatagcaatgcacagccaattt | |
| | | catgctccactatagattttcctggaaacatgaactgccaagagaggaatgggacacaaaacc | |
| | | aaacactgttttatatttatggtttgcaaactggcatttcatcagtggctaaattcacagatatccta | |
| | | tatagattgtatacagaactgagactgattttgtaccgattagaatgattgctatgatctttgagaa | |
| | | atttttctgcactatttgcactgaaatgtttatttattgttgataaattgtatcatatttaagttccactg | |
| | | ctgttcctcttaccttgattaaatgcctatgcatgtacttttagctagtttttaatattttataaaacttc | |
| | | atttaaatttgtatttttaacttgaagttccatttctttatcaaggatggtatttagatttttttcctcttaa | |
| | | ccttttttcaaaaactattttcaactgtgaggaaacccttatttttctttctttgtggataaaactttcaa | |
| | | aagcaatttaagatattcatagtgttaggaaacaccaaacctgcctatgtgccatctcacaaaag | |
| | | aaacttttaatacctacaataaatcaaaagaataaaccagctgttcttatatattgtttcatttttaaa | |
| | | actaaagatgcatttaagaagcaatacaagtaaatattttacctaataggaaaaaaaaaagttgc | |
| | | ctttcatttaaaccattccaacagaaattcttatgctaatttaaaacatatatatatctggtaggtttg | |
| | | tggttggataggttttctaaattcctaatgttaaaaacaatctttatgttaatatacactaaatctata | |
| | | cacaaaaaaagtcagtgaacttttctgacctttactgtgagttaccttttcctaagaggaaagcta | |
| | | tagtaataagtaaaatttaatttttaggcaatcctgatttttaatgaatttaattgagtgttcttgtatac | |
| | | tacattgagcagtttgcttctataccgtgtcacaaaattcatgtatttcttgagaagccctaaaagc | |
| | | tcataaaggaaaatgccgtgaactatgtagctcaggcttggtaaggtgccatctaaattacaaa | |
| | | acaaactaatgcataattttgcttaaatttcatcccagtatgattgtcttcccaacaccagcatata | |
| | | gtatagattgtctgtcttttttatattttttagttcttcctgtacatgtttttggcaataaagttataggaa | |
| | | gaacaaaattattttgttagaattaaaacatgcttaatatttagtctgtttgtggagggcaggtattc | |
| | | acgtggactgagatacaatgttggatacagaaaataactttcattgtcttcctgacactgtgctaa | |
| | | ggacatgctgttaaagcttcaaagtgaccagatgaggaaggaataattaattattactcctgattt | |
| | | gtagataactgaggtaagagtgtttcaaatttatgatagtcttttgggtattcagaaacctttcctta | |
| | | tactgcactggccaccagagcttaattttcccagcagttacagcaatgggagatagaacagtct | |
| | | caatcttttgccaaccatcaggttcctagaaaccaggtaggtgtatcccataacaagggagga | |
| | | gcataccacagcccctcatttgattaattcatttgatctatctatgttattaagtacctactaggaat | |
| | | aaggcattgtggaaatactatacaaagataaacattgtttagatgcttatctactttccttttcacca | |
| | | gaaaaacagaaaaaaaagaaacattttcttacagagtaaaaatgttctacataatcacatgagta | |
| | | gttcatctcagtgttttttattctttaaagttgaactatcccagtttcattctataccattcattggataa | |
| | | ccttgttacaacccagtcatgaaacagagcagtgtgatcagttatctgcatttaacaaatagaca | |
| | | aatcagtttacataaaggttatgtatgtcacccacgatgaaaagaatctgcatttgaatatgcccg | |
| | | tatgaatgtgggttctgtttttgcaacagagattaagtgaccattttttctaattttatggctatatattt | |
| | | tcttcataaaaattggtcacatcggagaagcagtgccacaggaaaaatgaaatgcatgtgaaa | |
| | | gtttgtattctgattttacaagatgagatagaaatcagaattaaagaggaatacttaggagttact | |
| | | aggctaatcagtgtacgaatttgtcataggtagagatttaaaggttaatatcttaaaatagaagaa | |
| | | aattctaaatcaatcaatcagtgagatataaactaaacagacccacttcaaagttgaaagaaatt | |
| | | tctaggcataaattgagactaggaaatttatatcagaatagagggtgcttgacacatatatatgct | |
| | | taaattgaaggacagctcagattcatttttaggagaagaaagtaaactaatgtgctcttaaagaa | |
| | | taaaaatttattctatggtttctgtctctgatcatcaccttccattctataaaaagctcagttactgatt | |
| | | tgctgggtcatggtcaaaattcttacctatttatttcatatcaactttaaaaaataaattacttgcatt | |
| | | ctatatattactaattgggaagtaatatgcctcaaatcagttttatactggattattccctatgcttta | |
| | | aaccactgctctcaataaaacacttcctgattaatgtttgattattagatattttagtcttgttgggga | |
| | | tattttagtcttgttgggttagccatgctctgaagaatctgtgaaagtacagtaaagttttaataag | |
| | | caataaatgtaaccttttatataaatctcagtgctaggttaacttctaataagcagacgaacatgtt | |
| | | acataaattataatgtctgtcttgtaaaaaagttgaggggactaaaagtttatgactctgatatgg | |
| | | aagttgtcatattaaaaaactacattttaaaacatcaaatatttatactatttgcttttcaaataaaag | |
| | | catagtgctgtttggcata | |
| PRRC2B | NM_013318.3 | gcagatcgggagcggtgccgagaaaaatttccttactagatgacatttcatcgcaatgtccgat | 42 |
| | | cgtttggggcaaattaccaagggcaaggatgggaaaagcaagtactcgactctcagcctgttt | |
| | | gataagtataaaggaaaatcagtagacgcgattagatcctcagttattcctagacatggcttaca | |
| | | gagtcttgggaaagttgctgcagcccggcgcatgccaccgcctgcaaacctgccaagcttga | |
| | | agtctgaaaacaaaggaaacgaccccaacatcgtgatagtacccaaggacgggacgggat | |
| | | gggcaaacaagcaggatcagcaagacccaaagagttccagtgcgacggcctctcagccgc | |
| | | cggagtcgctgccgcagccgggtttgcagaaatctgtctccaatttgcagaaaccgacacagt | |
| | | caatcagtcaggagaatacaaattcagtgccaggtggaccaaagtcatgggcacagctgaat | |
| | | ggaaagccagtaggacacgaaggtggtttaaggggctcaagccgactgttatccttctctccc | |
| | | gaggaatttccgacgctgaaagcagctggagggcaggacaaggctggcaaagaaaaggg | |
| | | cgtcttagatctgtcgtatgggccaggaccaagcctccgccctcagaatgtgacaagctgga | |
| | | gggagggcggtgggcgacacataatttctgccacgtctctgagcacctccccaactgagctg | |
| | | ggcagcaggaactcgagtacgggagatggagccccctcctcggcatgtaccagcgattcta | |
| | | aggacccctctctccgcccggctcagcctgtccgaaaaggggcttcacagttcatgggaaat | |
| | | gtataccacccacctacataccatgacatgcttcctgcttttatgtgttcgccgaagtcatcagaa | |
| | | aaccagggtacagtggaacgaggctcttttccccttcctcagctccgccttgaacctcgagttc | |
| | | cttttagacagttccagatgaatgaccaagacggaaaagaaaacaggctgggattgtctcgcc | |
| | | cactccgcccactaaggcagctggtggagcgggcaccacggcccaccattatcaatgcgga | |
| | | aaacctgaagggccttgacgatctggacgccgatgccgatgatggctgggcaggcctccat | |
| | | gaagaagtggactattctgagaaactgaagttcagtgatgatgaagaggaggaagaagttgt | |
| | | gaaggacggcaggccaaagtggaacagttgggaccctaggaggcagcggcagttgtcaat | |
| | | gagctctgcagacagtgcggacgctaagcggactcgagaggaagggaaggactgggctg | |
| | | aagcagtgggtgcgtcccgtgtggtccgaaaggcgccagaccctcagccaccgcccagga | |
| | | agcttcatggctgggcaccaggccctgactaccagaagtcatcaatgggcagcatgttccgg | |
| | | caacagtccatcgaggacaaggaggacaagcccccaccaaggcagaagttcattcagtcag | |
| | | agatgtccgaggcggtggagcgagcccgaaagcgccgggaagaagaggagcgccgagc | |
| | | ccgggaggagaggctggccgcctgtgctgccaaactcaagcagctggaccagaagtgtaa | |
| | | gcaggcacgaaaggcaggtgaggcccggaagcaggcagagaaggaagtgccctggtctc | |
| | | caagtgctgagaaggcatctccccaggaaaacggccctgctgtccacaaaggctccccaga | |
| | | attccctgcccaagagacccccaccacattcccagaagaggcacccacagtgtccccagca | |
| | | gtggcacagagcaacagcagtgaggaagaggccagagaggctgggtcccctgcacagga | |
| | | gttcaagtatcagaagtcccttcctccccgattccagcgccagcagcagcaacaacagcagg | |
| | | agcagctgtacaagatgcagcactggcagccggtgtaccccccgccgtcccacccccagcg | |
| | | caccttttacccacaccacccccagatgttgggcttcgatcccaggtggatgatgatgccttcct | |
| | | acatggacccacgtatcacgcccactcggaccccggtggacttctacccctccgccctgcatc | |
| | | cctcaggactgatgaagcccatgatgccccaggagtccctcaatgggacaggctgtcgctct | |
| | | gaggatcagaactgtgtgcccccactccaagaaagaaaagtgacccccatcgactcacccc | |
| | | ctgtgtggagcccagagggctacatggcactgcagagcaagggctacccgctcccgcaccc | |
| | | gaagtcgagtgacaccttggctatggacatgcgtgtcaggaatgaaagctctttctctgcctca | |
| | | ctcggaagggcagggggcgtaagtgctcagcgcgatctctttgaggagagaggggaggag | |
| | | tacttgagtgcttttgacaagaaggcccaagcagactttgacagctgtatctcttctcaaagaat | |
| | | aggccaggagcttttgtttccaccccaagaaaatgttcaggatgcaggtgctcctgggggtca | |
| | | cacccaaaacctcaggtgttccccattggagcctgactttgtcccagatgagaaaaagccaga | |
| | | gtgtggcagttgggatgttagccaccagccagagaccgctgacacagcccatggtgttgagc | |
| | | gggagacaccccgggaggggacggcctttaacatctcctcctgggacaagaacgggagcc | |
| | | ccaacaaacagccatcctcggagcctgaatggactcccgagccccggagctccagcagcc | |
| | | agcacccggagcagacgggcaggacccggaggtcgggacccatcaagaaaccagtcctg | |
| | | aaagccctcaaggtggaagacaaggagaaggagcttgagaagattaagcaggagctaggg | |
| | | gaggagagtacccggctggccaaggagaaggagcagagccccacggcagaaaaggatg | |
| | | aggacgaagagaacgatgcctctctggccaactcctccaccaccactttggaggacaaagg | |
| | | ccctggccatgccacttttggccgcgaggccaccaaatttgaagaggaggagaaacctgaca | |
| | | aggcctgggaagccagacccccacgagagtccagcgatgttccccccatgaagagaaataa | |
| | | ctggatctttattgatgaggagcaagcctttggggtcagaggacaggcccggggccggggc | |
| | | cgtggtttcagagagttcacttttcgtggtcggcctgctggcggaaatgggagcggcctctgtg | |
| | | gtgggggggtcctgggggcccgcagcatctactgcagcagtcagcgcagcggccgtggcc | |
| | | ggggcctgcgagagtttgcgcggccagaggactgccccagagccaagccccgacggaga | |
| | | gttgccagtgagacccatagcgagggctcagagtatgaagaacttcccaagcgccgccggc | |
| | | agaggggctccgagaacgggaatgaaggctcgctcctggagagggaggagagcaccttg | |
| | | aagaagggcgactgcagagattcttggcggtccaacaaggggtgctctgaggaccacagc | |
| | | ggtctagatgccaagagccgaggccctcgggcctttgggcgagccctccctccccggctga | |
| | | gcaattgcgggtatggacggagaaccttcgtctccaaagagtcaccccactggcagagcaaa | |
| | | agtccaggcagctcttggcaggaatatggcccttccgacacatgcggatcccggcgacctac | |
| | | agacagagactatgtcccagattcctacagacaccctgacgcatttggtggccggggctttga | |
| | | ggacagccgcgcggaggacaagagatccttcttccaagatgaacacgtggcagattctgaa | |
| | | aatgcagagaaccggcccttcaggagaaggcgccccccacgccaagataagccccctcga | |
| | | ttccggcgcctccggcaagagcgggagtccctgggcctgtggggacccgaggaggagcc | |
| | | ccacctgctggcaggtcagtggccaggcaggcccaaactgtgttctggggacaagagtggc | |
| | | actgtgggccgcaggtcccctgagctctcctaccagaactcctccgatcacgccaatgagga | |
| | | gtgggagacggcctccgaaagcagcgacttcagcgagcggcgggagcggcgggaaggc | |
| | | cctgggtccgagcccgactcccaggtggatggtggcctgtcgggggctagtttgggtgaga | |
| | | agaaggagctggccaagaggagcttctccagtcagagacccgtggttgacagacagagcc | |
| | | gaaagctggagccgggagggtttggggagaagcccgttaggccaggtggtggtgacacct | |
| | | cccctcgctatgagagccaacagaatgggacgcctttgaaagtgaaaagatccccagacga | |
| | | ggccttgcctggaggtcttagtggctgcagcagtgggagtggccactccccctatgccctgg | |
| | | agcgggcagcccatgccagtgctgaccttcccgaagcctccagtaaaaaggcagagaagg | |
| | | aggccaagttggctgctccgagggcaggtgaacagggagaggccatgaaacagtttgacct | |
| | | gaactatggaagtgccatcattgaaaattgcgggtccagccccggggaggagagtgaggtg | |
| | | ggttctatggtgggcgaaggcttcatcgaagtcctgaccaagaagcagcgccgcctgctgga | |
| | | ggaagagagaagaaagaaggagcaggccgtgcaggtgcctgtcaaaggtcgaggcctttc | |
| | | ctcccgtattcctcctcgatttgcaaaaaagcagaacaacttatgtctggagcaaggtgacgtg | |
| | | accgtgcctggcagcagcctgggcactgagatctgggagagcagcagccaggctctccctg | |
| | | tgcaggccccagccaacgactcctggaggaaagctgtcactgccttcagcagcaccgagac | |
| | | tggctctgcggagcagggttttaagagcagccagggagatagtggcgttgacttgagtgccg | |
| | | agtctcgggagtcgtctgcgacctcctcgcagcgcagctccccatatgggactctgaagcca | |
| | | gaggagatgagcgggcccggcctggcggaacccaaggccgacagccacaaggagcagg | |
| | | ctccaaagccatctgagcagaaggattcagaacaaggctctggacagagcaaggagcaca | |
| | | gaccaggacccatcggcaacgagcgttctctgaaaaacagaaagggctcggagggggcc | |
| | | gagcggctgcaaggggctgtcgtcccgcctgttaacggggtggagattcacgtggactccgt | |
| | | gctgcctgtgccacccattgaatttggagtcagtccaaaagactccgatttcagcttgccacct | |
| | | ggttctgcctctggtcctactgggagtccagttgttaaacttcaggatgccttggccagtaatgc | |
| | | agggttaacacagagtatccccatcctgcggcgggaccatcacatccagagggccatcggtc | |
| | | tctccccaatgtccttccccaccgccgaccttactctgaagatggagtctgcgcgcaaggcttg | |
| | | ggaaaactcccccagtttgccggagcagagctctccaggcggcgctggctcaggcatccag | |
| | | cctccatcctctgtgggtgcctccagcggggtcaactacagctccttcggtggagtgtccatgc | |
| | | cacccatgcctgtggcctctgtagcaccttctgcttctatgccaggcagccacctcccgcccct | |
| | | gtacctggatggccatgtgtttgcaagtcagccccggctggttcctcaaacgatacctcagca | |
| | | gcagagttaccaacaggccgccgctgcccagcagatcccgatctcccttcacacatctctgca | |
| | | ggcacaagctcagcttggactgaggggtgggcttcctgtgtcccagtcccaggagatcttca | |
| | | gctccttgcagcccttcagatctcaggtgtacatgcaccccagcctgtcaccgcccagcacca | |
| | | tgatcctctctgggggcacagccttgaagcctccatactcggcgttcccaggcatgcagccct | |
| | | tggagatggtgaagccgcagtctggctcaccctaccagcccatgagcgggaaccaagccct | |
| | | ggtctacgagggccagctcagccaggctgctggcctgggtgcctcccagatgttggactccc | |
| | | agctcccacagctgaccatgccactgcctcggtacggctccgggcagcagccactgatcctg | |
| | | ccccagtctattcagctgccacctgggcagagcctctccgttggggccccccgaaggattcct | |
| | | ccgcccgggtcccagccgccagtcctgaacaccagcagagagccctctcagatggagatg | |
| | | aaaggcttccactttgccgacagtaaacagaatgtcccttcaggaggccccgtgccatcgcca | |
| | | cagacctacaggcctagctctgctagccccagtgggaagccctctggatcagcagttaacat | |
| | | gggctctgtgcagggacactacgtgcaacaggcaaaacaacgagtggatgagaaacccag | |
| | | cctgggagccgtgaagctgcaggaggccccctcggctgcctcccagatgaagcgaaccgg | |
| | | agcgatcaagcctcgggctgtcaaagtggaggagagtaaggcctgacagtgcctggctgcc | |
| | | acctcgcctctccctactgaggacggtgccgccatgcggcctcgacacagccgacactcgg | |
| | | gagcctcaccagatccaccgtccaaatgcgtggcccagactgagagacctccctcctctcca | |
| | | ctcccgaaagctccgttgtcaaccagcttgcacccgtggatatatggcattgacccgcttgcttt | |
| | | gatacgaaacaaaaaagcagacgactccttcatcccatctgctcctaccgtgactgtggagtg | |
| | | acgcctcctgtgcagtgcagatttgccctccctgcctcctccctgtcctgccgcgcagccagg | |
| | | gcgccttctcagcagtgcttccggcccagccgcccatccctaggcacagtgatttggcagca | |
| | | gggtcattttactttgaggctttttgttttaaaatgtagccaaggtttttacaaaggggaaaggaaa | |
| | | agaaaacaaaaacgcaagctccatgtgtatagctgaacttttatatgtttcttgccagcccctcc | |
| | | gctcccttccatctctagcctctgtcctgtttagtttgatacgtcactgcagtaccttaagaggtga | |
| | | ctcttaagaatgcatcccctcctgattcctcagctggttcacccttgaggttatttgcaaaaagaa | |
| | | aaggaggttcttgagggcaccgattgcgagcattctggtgcctggctccccgcctgggaagc | |
| | | gatggggtgctcagagcagcaggcaggttgggggagggggggggtcatagttgggttcca | |
| | | gctcctggcttgatgagcccagggcgcttacaggcagcccatgaagttgatgacagttttagc | |
| | | atgagaatcacacagggtccctgtcctgggctcctctaaagccagtggatgtgctgggcacc | |
| | | agagacaaatcatggagatggctgctggtggctcccaggttggcccagatggggtgagctg | |
| | | acataccacaggcccatcccaggccccgtgggctctgcttctggggctccataccctgccctg | |
| | | caggggtgctgtgtttttcacacatttctttccctgaagccttctgtaacctgtcattttccttccttc | |
| | | ctcttccggagcctgctgctttctctggacctgtctccacctcccacacagctcatcgtgaacac | |
| | | cacttggtgatggagggagtggacccgtgtgtggtccccaagtgaggccactgggagtttgt | |
| | | ccttttcctcctttgcttcactcccagcagcagacccaggttgtcaggacaggagggcctgag | |
| | | ctaagcagtaggcatcagtctcgtttgtcttcagacggcgggggcaggtccagggtgaggct | |
| | | gggtggagggctgaccaaggtccaaagggcctgcgcagcctccgggagggcagcttctcc | |
| | | agccagaggcttgtgtgagccatcgtgtgctgggcttgtttttaagtaagaaacaaggaaatca | |
| | | ctccagattctgtcattccaaggaaagggaaggggacagttcaggtttctcagctgttcttagg | |
| | | ggtcactgagcgtctacctcctcctccagaggaggctggctcagaacacctagaggagggg | |
| | | gccggggatgcaccccccaccagaggctgccttcagcgtctcacgggtgcaggacagcgc | |
| | | tcaggcttgggctctaagctctgtgtctagtgtagaacatggggaaggagcatcttaggaactg | |
| | | ctgaagtaacttcttactgctctcacaattctaaggaagcgggagaacggcctcctaccaacag | |
| | | cgcccaccccagagctgcctgggaaagggcagttttactgaaaggtgctttactgttcacctg | |
| | | catctttcagcagctcccctcctgccctcacctggtcttttccctctttatcccaagcctttatgctt | |
| | | gagtcccttccccaggggctgcccacccgacagttccaggcattccctacctgagcttcttgtc | |
| | | tgcttttccttctcccactgcaagcggctgcttgtggggcctgggatgagccctctctgtcccca | |
| | | ccggccctccttgccaagccattcctgggtgagttcaggcctgcgggagccacacattcatct | |
| | | ccacctggacacttgagccgcatggccagacccctcccacctgatgcggtggtgcgtgtgat | |
| | | ttgtcaaaagaaagccttctggatgctgttaagatgtacccttcaggtgaacctggtatcagacc | |
| | | cacagtacttgctgtttgagaaaaaataaaaacaaaaaggtcacctgttctccagcccttttctct | |
| | | tacctggtatttccttcctttctcctcccccaccccaaataaaaaaacaaaaaacactagaatttat | |
| | | ttatatgtattgatgttgtaggtctaggtgaaaaaaaaagaagtaaatgtttcactgctctatttata | |
| | | tataatgtctgaattaattctgtgcaggaaaggccaggaaattgcatgtgaagttcggtgcagtc | |
| | | accacctgtgtgtgacctgagctgcagtctcttcgctgagatgcaggttttaaatgagacttggg | |
| | | gggctgagggcaggcctcaggcctcccagcgccccaacccctccttggtctaatgaaatgca | |
| | | gttcttagtgcagagatgttttaaggtgcaatatatctcttcctttcccgtggttttagagccaagct | |
| | | caaggtagtaggacgtagggtcttattttgttttcaaacccccatcctcagagcgcagatacatg | |
| | | cagaggcttctgccaggataccacggggccttagtgggaacaggtggagaccagcacttcc | |
| | | ctttcctgctgctgaggtagggattggggggtcagaacccactcacttttgcctgttaaagttgc | |
| | | cctcctgacgctggcagctctgccttggtcactggggatgcggctcgttgctcagccaccagt | |
| | | ggccttgcggtattgtccaccatccactagagtgggatgaagtccagagtgtgggtatacatct | |
| | | cagatgcccatctacccactggggacttcaatgccagctgcatttggtttggttttcttaactgttg | |
| | | gcttctccccacagcgttttttgtttttttttaaacattcatattgttttcaaacttggaattcatagaca | |
| | | ctctggctctaggttccttaagggggaaaacaaaagatgactttatttcacattcaagaaaatca | |
| | | gttcagttccaaagctgtggtccttccagccacttctagggacactggggaaccttgttaaacgt | |
| | | tgacatcagtgctctccagccgtgctgtcaccctcctatcttctggatctgccttcgcgatggtca | |
| | | gtgacagcttctggaagctgagcacacacaggtgcacagccatgctgtggtctggcctgcta | |
| | | cggcagcatggcagctctggtggagccttctcccttgccatttggttcccctgtgccaagtagc | |
| | | tgcaggctgcccctcaaatcttcatttgtcccttttcacttcctgcagaacaagcctgggttagag | |
| | | ggtctgctggaaatggcctttgaagaccaaggataccaggatgtgtgcactctgtcgtgttctg | |
| | | tgatgaatgggaaacgtaggcttccagaaagccagctctcttctgaaatgtgacggacctaag | |
| | | caggaagtcatccaggacaggagtggctcagtgttggggatggacgctgtcgcccagccat | |
| | | gctccaccagggccaccaatgtgtagttggctggtggtcttcgggcatgtgagacctgctcttc | |
| | | actgtttccaccccacttggtggcctccaggatggtagtggcaccctcagagccccatcttcag | |
| | | catgttctgaagcctcagagtggaaattcctgctaaggctctgtgtggacgcctttctcccgtga | |
| | | tctaaaggggacactgtactcaagcttttgacctcatgccttgtgtagtaaaaaaggatttgggg | |
| | | gttttgtttggttcctgagagggttgtgttttgtttttgtttccttttgtttatgttttggcctttcctctttg | |
| | | tctttccatgtagaccagatatttgaaagggcagacgatggctagaggtgtaatgtgcagcttgt | |
| | | ttatacggtattttgggaaacttaccttggatgggaaatcgaatcgtggattcaccaggccggtg | |
| | | ctggcacactcaccctcgccctttccctccggttcagtacctattgtttctcctttcaaatatgtgat | |
| | | tgtactagctctttccatatgaaagaattctccttatttaaataaaaaaagtttaaaaa | |
| DOPEY2 | NM_005128.3 | tcccacagtgcctggcccagaagccttgctaaatatttgaacaggattgcccaatacttttctgct | 43 |
| | | gtgagaatgtaagatggatccagaagagcaggagctcttaaatgattacagatacagaagcta | |
| | | ctcttcagtgattgaaaaggctttgagaaattttgagtcctcgagtgaatgggcggatctcatatc | |
| | | ttcacttggcaaactcaacaaggctcttcagagtaacctgaggtactccttgttgccaagacgg | |
| | | ctcctcatcagcaaaagattagctcagtgtttgcaccctgccctgcccagtggtgtccacttaaa | |
| | | agctctggaaacctacgagattatctttaaaatcgtggggaccaaatggctggccaaggactt | |
| | | gtttctgtacagctgcgggttatttcctctcctggcacacgcggcggtgtcggtgaggccggtg | |
| | | ctgctcaccctgtacgagaagtacttcctcccactgcagaagctgctcctgcccagtctgcag | |
| | | gccttcatcgtgggcctgctgcccggccttgaagagggctccgagatctccgacagaacgg | |
| | | atgctctgctcctgagactgtcgctggtggttggcaaagaggtgttttacaccgccctctgggg | |
| | | gagcgtcctggccagcccgtccatccgcctccctgcctcagtcttcgtggtgggccacatcaa | |
| | | cagggatgcccccggccgggagcagaagtacatgctggggaccaatcaccaactcacggt | |
| | | gaagtctttgcgtgcctccctgttggactcaaatgttcttgtgcaaagaaataatctggaaatcgt | |
| | | tctgtttttcttcccattttatacctgtctggattccaatgagagagccatccccctcctcagatctg | |
| | | acatcgtgcgcattctctcagccgccacccagaccctactgagaagggacatgtccctgaac | |
| | | agaagactgtatgcatggttactaggctcagacataaaaggaaataccgttgtgccagaatct | |
| | | gaaatctcaaattcttatgaagaccagtcgtcttatttttttgaaaaatactccaaggatcttttagtt | |
| | | gagggtttggctgagatattgcatcagaagttcatagatgctgacgtggaggaacgccatcat | |
| | | gcatacctgaagccttttcgcgtcctcatcagtctgcttgacaagccagaaatagggcctcaag | |
| | | tggttgggaatttgtttctcgaagtcatcagggccttttattcttactgcagagatgcccttggctc | |
| | | tgatcttaaacttagctacacccagagtggaaattcgctgataagtgcaatcaaggaaaacaga | |
| | | aatgcctctgagattgtcaaaacggtaaatttgctgataacttctctaagcacagactttctctgg | |
| | | gattatatgacaaggtgttttgaggaatgctttagaccagtgaagcagcgttacagcgtgagga | |
| | | acagcgtcagccctccccccacggtctcggagctctgcgccctcctggtcttcctgctggatg | |
| | | tcattcctttggaactttactctgaggtgcaaacccagtatctccctcaggtgctcggctgcctg | |
| | | gtgcagcctcttgctgaggacatggaggccttaagtttacctgaactcacgcatgccttgaaga | |
| | | cgtgtttcaaggtgctcagcaaagtccagatgcctccttcctacctcgacacggagtccacca | |
| | | gcggaacctcgagtccagtaaaaggtgaaaacggcaaaataattttggaaacaaaggcagtg | |
| | | attcccggtgacgaagatgcttcgtttccccctctgaagtctgaggacagtgggatcgggctc | |
| | | agtgcctcgtcaccggagctctctgagcacttgagggttcctcgagtttctctggaaagggac | |
| | | gacgtttggaagaagggcgggagcatgcagaggacgtttctttgcatccaagagctaatcgc | |
| | | caactttgccagcaagaacatttttggagtacagctgacagcgtcaggagaagaaagcaagt | |
| | | ccgaggagcctgcagggaagagggacagggatgggacgcagagcctggcagccaatgat | |
| | | tccagcaggaagaactcttgggagcccaagcccatcactgtgcctcagttcaagcagatgct | |
| | | gtcagacttgttcacagcacgagggtctccattcaagacaaaaagttcagagtcaccatcgtct | |
| | | tcgcccagcagccctgccaggaaaaacgggggagaatgggatgttgagaaggtggtcattg | |
| | | acctggggggttccagggaggaacgcagggaggcctttgccgccgcctgccacctgctgct | |
| | | ggattgtgccactttccctgtctacctgtccgaggaagagaccgagcagctctgtgcaacgct | |
| | | cttccagctgccaggagccggtgattccagttttccatcttggctgaagtccctcatgactatttg | |
| | | ctgctgtgtgactgactgctacctccagaacgtggccatttccactctgctggaagtgataaac | |
| | | cattcccagtccctggcgcttgtcattgaagacaagatgaaacgctataagagctctggacac | |
| | | aaccctttttttggcaagctgcagatggtgacggttcctcccattgctccagggatattgaaagt | |
| | | cattgcagagaaaacagatttctatcagagggtggctcgtgtgctttggaatcagctgaacaaa | |
| | | gagacccgggagcatcacgtcacctgcgtagaattgttctaccggctgcactgcctggcccct | |
| | | acggccaacatctgcgaggacatcatctgccatgccctcctggaccctgacaagggaacaa | |
| | | ggctggaagctctgtttagattttccgtgatctggcatctgacaagagagatccaaggcagtcg | |
| | | agtaacatctcacaatcgctcctttgataggtccttgtttgtcgtgctggacagcctggcctgca | |
| | | cggatggtgccatcggtgcggcagcccagggctggctggtgcgtgcgctctccctcgggga | |
| | | cgtggctcgcatcctcgaacccgtgctcctgctgctgctgcagccaaaaacccagagaacct | |
| | | ccatccactgcctcaagcaggagaactcggccgatgacttgcaccgttggtttaacaggaag | |
| | | aaaacctctttcagagaggcatgcgcagtgcccgagcctcaggagagcggctctgaagagc | |
| | | acctgcctctgagccagttcaccacagtggaccgtgaagccatttgggccgaagtggagaag | |
| | | gagcccgagaagtacccgctgcgaggcgagctgagcgaggaagagctgccctactacgtg | |
| | | gagcttccagacaggacggcccacggcgccccggacagcagcgagcacaccgagtctgc | |
| | | agatacaagctcctgccacacggacagcgagaacacgtcctccttctcctccccttcccacga | |
| | | cctgcaggagctgagcaacgaagagaactgctgtgcacccatccccatggggggcagggc | |
| | | gtaccccaagcgctcggccctgctggcggccttccagtcagaaagcttcaaggctggggcc | |
| | | aagttaagcctggtgcgggtggactcggacaagacgcaggcttctgagtcgttctccagcga | |
| | | cgaggaggcggacttggagctccaggccctcaccacatccaggctgctaaagcagcagcg | |
| | | ggaaaggcaggaggccgtcgaggccttgttcaagcacatcctgctctacctgcagccctacg | |
| | | actctcggcgggtcctctatgccttctcggtgctggaggctgtgctcaaaaccaaccctaagg | |
| | | aattcatcgaggctgtgtccaggactagcatggataccagctccaccgcgcacctcaacctca | |
| | | tctccaacctcctcgctcgccaccaggaggccctcattggccagagtttctacggaaagctcc | |
| | | agacccaggtccccaacgtgtgcccccactctctgctcctggagctgctcacctacctctgcct | |
| | | gagcttcctgcgctcctactacccttgctatttgaaggtctcgcaccgagacattctcggcaacc | |
| | | gggacgtgcaggtcaaaagtgtcgaggttttgatcaggataatgatgcagctggtctcagtgg | |
| | | ccaagtcttcggaagggaagaacgtggagttcatccacagcttgctgcagaggtgcaaagtt | |
| | | caggagtttgtcctgctctccctgtcggcgtccatgtacacgagccagaagcgctacgggctg | |
| | | gccaccgcccaccacggcagggccctgccagaggacagcctctttgaggagagtctcatta | |
| | | acttgggtcaggaccagatctggagtgagcacccgctgcagattgagctgctgaagctgctg | |
| | | caggtgctgattgtcttggaacaccacctgggtcgggcccatgaggaggcggaaaaccagc | |
| | | ccgacctgtcccgggagtggcagagagccctgaacttccagcaggccatcagcgccctgca | |
| | | gtacgtgcagccccaccccctcacctcccagggtcttctggtctctgcggtggtgaggggtct | |
| | | gcagcccgcctacggttacggcatgcatccggcctgggtgagcttggtcacgcattccttgcc | |
| | | ctacttcggaaagtccctgggctggacggtgacaccctttgttgtccagatttgcaaaaacttg | |
| | | gatgacttggtcaagcagtatgaaagcgaatctgtgaagctctctgtcagcacaacctccaag | |
| | | agggaaaacatttctccagattatccactcacccttctagaaggtctaacgaccattagtcattttt | |
| | | gtcttttggaacaagccaaccaaaacaaaaagaccatggctgcaggtgatcctgccaacttga | |
| | | ggaatgccagaaatgccattttggaagagctgcctcgaactgttaacaccatggcccttctctg | |
| | | gaatgttctcagaaaggaggagactcaaaagagacctgtcgatctcctaggggccacgaag | |
| | | ggatcctcttccgtttactttaaaaccaccaaaaccataagacaaaaaattttagacttcttaaac | |
| | | cccttgacggcccatcttggggttcagttgacagcggctgttgcggcagtgtggagcagaaa | |
| | | gaaagcccagcgtcacagtaagatgaagattatcccaacggcaagtgcatcccagctaaccc | |
| | | ttgtcgacttggtgtgtgcactcagcaccctgcagactgacacgctgctgcacctggtgaagg | |
| | | aggtggtgaagaggccaccccaagtcaaagggggtgatgagaaatcgcccctagtggacat | |
| | | tcctgtgttgcagttttgctatgcttttctccaaaggctcccagtaccagccttgcaagagaacttt | |
| | | tcttcactgttgggagtattgaaagagtctgtacagttgaatctagccccacctgggtattttctg | |
| | | cttctcagcatgctgaatgactttgtaacaagaactcccaacctggaaaacaagaaggaccaa | |
| | | aaagacctgcaggaaatcactcagaaaatcctagaagctgtggggaacattgccggctcttcc | |
| | | ttggagcaaaccagctggctaagcagaaacctggaagtgaaggcccaacctcaggcctctct | |
| | | agaagaatctgatgctgaggaggacctgtatgatgctgctgcagcttcagcaatggtgtcttca | |
| | | tccgccccgtcggtgtacagcgtgcaagccctctctctcctggcagaggtactggcttccctc | |
| | | ctggacatggtttatcgaagtgatgagaaggagaaagctgtgccgttaatctcccgtctgcttta | |
| | | ctatgtttttccatacttacgcaaccacagtgcctacaatgctcccagcttccgggctggcgctc | |
| | | agctgctgagctccctgagtggctatgcctacacaaagcgagcctggaggaaggaggtcct | |
| | | ggagctgtttctcgaccccgctttctttcagatggatacttcctgtgttcattggaagtccattattg | |
| | | accatcttttgactcatgagaaaacaatgtttaaggatttaatgaacatgcagagcagttctttga | |
| | | aactattctcaagttttgaacagaaagccatgctgttaaagcgccaggcttttgctgtcttcagtg | |
| | | gagaacttgatcaataccacctttaccttccactgatacaagaacgcctgacagacaatctcag | |
| | | agttggacagacatccatagttgctgctcagatgtttctttttttcagagttttgctgctaagaatat | |
| | | ctcctcaacatttgacttcattgtggccaataatggtctctgaattgattcagacattcacacagct | |
| | | tgaagaagatctaaaagatgaagatgagtcattgagaagcaccaacaaagtaaacagaacga | |
| | | aagtttcagtcccggatgcaaatggaccctcagtgggggagataccccagagtgaactcatct | |
| | | tgtatttatcagcttgcaaattcttggacacagcgctttcttttccacctgacaagatgccattattt | |
| | | caaatttataggtgggcatttattccagaagtggacacagagggccctgccttcctgtcggatg | |
| | | tagaggagaatcaccaagaatgcaaaccccacactgtcaggattctagaacttctaaaattaaa | |
| | | gtttggggaaatcagtagctctgatgagatcaccatgaagagtgaattcccgcttctgcgccaa | |
| | | cattctgtttccagcatcaggcagttgatgccattcttcatgactctaaatggtgcatttaagaccc | |
| | | agagacagctgcctgctgatagcccaggaactccattcttggactttcctgtcacagatagccc | |
| | | aaggatcttaaaacaactggaagaatgcatcgaatatgattttctggaacatccagaatgttaac | |
| | | catgtgagagagaatatgtttaatccatgtattggtactttactgaaaaccaggttatattctaaag | |
| | | aagaaagaaggcaggatagtgcttttgaacaagcctatttccattttgaaagtagatttcaggct | |
| | | aggtgcggtggctcacacctgtaatctcagcactttgggaggccaaggcaggcagatcactt | |
| | | gaggtcaggagttcgagaccagcctgaccaacatggtgagaccctgtctctactaaaaataca | |
| | | aaaattagctgggtgtggtggcggcgcctgtaatcccagctacttgggaggctaaggcatga | |
| | | gaattgcttgaacccaggaggtggaggctgcagtgagccgagatcacgacactgcactcca | |
| | | gctgtgtgacagaatgagaccatctccaaaaaaaaaaaaaagtagatttcagataatttactgtt | |
| | | cagcaacaggacacacctccctaaatgccttgtaatatatttgaatctgattctgcatttcttcctc | |
| | | aatttatgtaatgaaaataaaattaatatatcatctaacagtagcacaaaatttgtaatatgaagta | |
| | | aagtatgaagataatgaagaagttgttttctttgttgaagcagttatatgggtctttctcagtatattt | |
| | | ctcttttctctaaaagtttaaacttattaaaagaatgttatttttaacctttcaaaaaaaaaaa | |
| NDUFB11 | NM_019056.6 | gctctggccggccccggcgattggtcaccgcccgctaggggacagccctggcctcctctga | 44 |
| | | ttggcaagcgctggccacctccccacaccccttgcgaacgctcccctagtggagaaaagga | |
| | | gtagctattagccaattcggcagggcccgctttttagaagcttgatttcctttgaagatgaaaga | |
| | | ctagcggaagctctgcctctttccccagtgggcgagggaactcggggcgattggctgggaa | |
| | | ctgtatccacccaaatgtcaccgatttcttcctatgcaggaaatgagcagacccatcaataaga | |
| | | aatttctcagcctggccgaaaatggttggccccacgaagccacgacaactggaggcaaaga | |
| | | gggttgctcaacgccccgcctcattggaaaaccaaatcagatctgggacctatatagcgtggc | |
| | | ggaggcggggcgatgattgtcgcgctcgcacccactgcagctgcgcacagtcgcatttctttc | |
| | | cccgcccctgagaccctgcagcaccatctgtcatggcggctgggctgtttggtttgagcgctc | |
| | | gccgtcttttggcggcagcggcgacgcgagggctcccggccgcccgcgtccgctgggaat | |
| | | ctagcttctccaggactgtggtcgccccgtccgctgtggcgggaaagcggcccccagaacc | |
| | | gaccacaccgtggcaagaggacccagaacccgaggacgaaaacttgtatgagaagaaccc | |
| | | agactcccatggttatgacaaggaccccgttttggacgtctggaacatgcgacttgtcttcttctt | |
| | | tggcgtctccatcatcctggtccttggcagcacctttgtggcctatctgcctgactacaggtgca | |
| | | cagggtgtccaagagcgtgggatgggatgaaagagtggtcccgccgcgaagctgagaggc | |
| | | ttgtgaaataccgagaggccaatggccttcccatcatggaatccaactgcttcgaccccagca | |
| | | agatccagctgccagaggatgagtgaccagttgctaagtggggctcaagaagcaccgccttc | |
| | | cccaccccctgcctgccattctgacctcttctcagagcacctaattaaaggggctgaaagtctg | |
| | | aaaaaaaaaaaaaaa | |
| ND4 | NC_012920.1 | atgctaaaactaatcgtcccaacaattatattactaccactgacatgactttccaaaaaacacata | 45 |
| | | atttgaatcaacacaaccacccacagcctaattattagcatcatccctctactattttttaaccaaa | |
| | | tcaacaacaacctatttagctgttccccaaccttttcctccgaccccctaacaacccccctcctaa | |
| | | tactaactacctgactcctacccctcacaatcatggcaagccaacgccacttatccagtgaacc | |
| | | actatcacgaaaaaaactctacctctctatactaatctccctacaaatctccttaattataacattca | |
| | | cagccacagaactaatcatattttatatcttcttcgaaaccacacttatccccaccttggctatcat | |
| | | cacccgatgaggcaaccagccagaacgcctgaacgcaggcacatacttcctattctacaccc | |
| | | tagtaggctcccttcccctactcatcgcactaatttacactcacaacaccctaggctcactaaac | |
| | | attctactactcactctcactgcccaagaactatcaaactcctgagccaacaacttaatatgacta | |
| | | gcttacacaatagcttttatagtaaagatacctctttacggactccacttatgactccctaaagcc | |
| | | catgtcgaagcccccatcgctgggtcaatagtacttgccgcagtactcttaaaactaggcggct | |
| | | atggtataatacgcctcacactcattctcaaccccctgacaaaacacatagcctaccccttcctt | |
| | | gtactatccctatgaggcataattataacaagctccatctgcctacgacaaacagacctaaaatc | |
| | | gctcattgcatactcttcaatcagccacatagccctcgtagtaacagccattctcatccaaaccc | |
| | | cctgaagcttcaccggcgcagtcattctcataatcgcccacgggcttacatcctcattactattct | |
| | | gcctagcaaactcaaactacgaacgcactcacagtcgcatcataatcctctctcaaggacttca | |
| | | aacttactcccactaatagctttttgatgacttctagcaagcctcgctaacctcgccttacccccc | |
| | | actattaacctactgggagaactctctgtgctagtaaccacgttctcctgatcaaatatcactctc | |
| | | ctacttacaggactcaacatactagtcacagccctatactccctctacatatttaccacaacaca | |
| | | atggggctcactcacccaccacattaacaacataaaaccctcattcacacgagaaaacaccct | |
| | | catgttcatacacctatcccccattctcctcctatccctcaaccccgacatcattaccgggttttcc | |
| | | tctt | |
| MORF4L1 | NM_001265603.1 | cggcgtgccctggggcggcgcgggcgcaggggcgcgtgcgcggcgggctgtcgttggct | 46 |
| | | ggagcagcggctgcgcgggtcgcggtgctgtgaggtctgcgggcgctggcaaatccggcc | |
| | | caggatgtagagctggcagtgcctgacggcgcgtctgacgcggagttgggtggggtagag | |
| | | agtagggggcggtagtcgggggtggtgggagaaggaggaggcggcgaatcacttataaat | |
| | | ggcgccgaagcaggacccgaagcctaaattccaggaggttgggatgaatgggttccggag | |
| | | agcagagtactcaaatacgtggacaccaatttgcagaaacagcgagaacttcaaaaagccaa | |
| | | tcaggagcagtatgcagaggggaagatgagaggggctgccccaggaaagaagacatctg | |
| | | gtctgcaacagaaaaatgttgaagtgaaaacgaaaaagaacaaacagaaaacacctggaaa | |
| | | tggagatggtggcagtaccagtgagacccctcagcctcctcggaagaaaagggcccgggta | |
| | | gatcctactgttgaaaatgaggaaacattcatgaacagagttgaagttaaagtaaagattcctg | |
| | | aagagctaaaaccgtggcttgttgatgactgggacttaattaccaggcaaaaacagctcttttat | |
| | | cttcctgccaagaagaatgtggattccattcttgaggattatgcaaattacaagaaatctcgtgg | |
| | | aaacacagataataaggagtatgcggttaatgaagttgtggcagggataaaagaatacttcaa | |
| | | cgtaatgttgggtacccagctactctataaatttgagagaccacagtatgctgaaattcttgcag | |
| | | atcatcccgatgcacccatgtcccaggtgtatggagcgccacatctcctgagattatttgtacg | |
| | | aattggagcaatgttggcttatacacctctggatgagaagagccttgctttattactcaattatctt | |
| | | cacgatttcctaaagtacctggcaaagaattctgcaactttgttcagtgccagcgattatgaagt | |
| | | ggctcctcctgagtaccatcggaaagctgtgtgagaggcactctcactcacttatgtttggatct | |
| | | ccgtaaacacatttttgttcttagtctatctcttgtacaaacgatgtgctttgaagatgttagtgtata | |
| | | acaattgatgtttgttttctgtttgattttaaacagagaaaaaataaaagggggtaatagctcctttt | |
| | | ttcttctttctttttttttttcatttcaaaattgctgccagtgttttcaatgatggacaacagagggatat | |
| | | gctgtagagtgttttattgcctagttgacaaagctgcttttgaatgctggtggttctattcctttgac | |
| | | actacgcacttttataatacatgttaatgctatatgacaaaatgctctgattcctagtgccaaaggt | |
| | | tcaattcagtgtatataactgaacacactcatccatttgtgcttttgtttttttttatggtgcttaaagta | |
| | | aagagcccatcctttgcaagtcatccatgttgttacttaggcattttatcttggctcaaattgttgaa | |
| | | gaatggtggcttgtttcatggtttttgtatttgtgtctaatgcacgttttaacatgatagacgcaatg | |
| | | cattgtgtagctagttttctggaaaagtcaatcttttaggaattgtttttcagatcttcaataaatttttt | |
| | | ctttaaatttcaaagaacaaaaaaaaaaaaaaa | |
| MRPL19 | NM_014763.3 | gtagtcttgacgtgagctagctggcatggcggcctgcattgcagcggggcactgggctgcaa | 47 |
| | | tgggcctaggccggagtttccaagccgccaggactctgctccccccgccggcctctatcgcc | |
| | | tgcagggtccacgcggggcctgtccggcagcagagcactgggccttccgagcccggtgcg | |
| | | ttccaaccgccgccgaaaccggtcatcgtggacaagcaccgccccgtggaaccggaacgc | |
| | | aggttcttgagtcctgaattcattcctcgaaggggaagaacagatcctctgaaatttcaaataga | |
| | | aagaaaagatatgttagaaaggagaaaagtactccacattccagagttctatgttggaagtatt | |
| | | cttcgtgttactacagctgacccatatgccagtggaaaaatcagccagtttctggggatttgcat | |
| | | tcagagatcaggaagaggacttggagctactttcatccttaggaatgttatcgaaggacaaggt | |
| | | gtcgagatttgctttgaactttataatcctcgggtccaggagattcaggtggtcaaattagagaa | |
| | | acggctggatgatagcttgctatacttacgagatgcccttcctgaatatagcacttttgatgtgaa | |
| | | tatgaagccagtagtacaagagcctaaccaaaaagttcctgttaatgagctgaaagtaaaaatg | |
| | | aagcctaagccctggtctaaacgctgggaacgtccaaattttaatattaaaggaatcagatttga | |
| | | tctttgtttaactgaacagcaaatgaaagaagctcagaagtggaatcagccatggcttgaatttg | |
| | | atatgatgagggaatatgatacttcaaaaattgaagctgcaatatggaaggaaattgaagcgtc | |
| | | gaaaaggtcttgattctgagaatgaatttggttagttgcagaagatacattggctctaagaggat | |
| | | atattttgagaccaatttaatttcatttataagaacatagtaattaagtgaactaagcattcattgtttt | |
| | | attaatactttttttctaaaataaaacttgtacaccagtttattactctaaaaagagaattacacatgc | |
| | | caaatggaccaatgtccatttgcttattggaggcaaagctacaatagaagtcagagcatcacca | |
| | | gaatggtctttaatgagcatggaacctgagcaaagggaataggtgggatgaattttttttttaatt | |
| | | gtgaaacaattcataagcacaatatgatttacagaataataaacattcatgtacccactatcaggt | |
| | | taagaaatagaacatttattaatatgtaggaatgttaagaaataaaacatttaataagatctcaga | |
| | | agactccagtaaatctgcaattgtatctctctcctttttaaatgtaaatatcatcttgacttgttaatta | |
| | | ttcccttgcatttcttttagtttactgccaacacatatattcttcaacaatatatttaattttgaaaaacc | |
| | | tgaaaaaaaaaacctgttagcaagtataaaggggcagtattactattattgcatgaaggcttcaa | |
| | | gggaaacgttacagtctttgggtcatagtctggcttcagcttcctctgagagtttacagaggcca | |
| | | attttgagcaaattcatggctaaggttatgagtgagttctgctaaacagaaggctcaccacaag | |
| | | gtatctggcaggattatactgggtagctggatgttgcagaaatgtggttagaggaagtaaactg | |
| | | ttttttgatgctcacagcatgatgaatcaaactctgtatcttaggattaggttaaaacaatacctttg | |
| | | gtatgatatgagtgttgttgctgatccatgcagcatggattggaaagctggggtataagcacac | |
| | | atgctaaagaaaaacatgtaatttggtccatactcacctggatatactgttcctcaggttaaaaaa | |
| | | tacagtactatcctaaatcttgaaggcaactctcagcctatccattgagttaccttcagatctgcc | |
| | | ctctggttcctagctgtcttgggactaacttctttcctgcgctcagctgttttctggattccatgtttt | |
| | | ccattttattgagtactaacttgttttgctgcagcacatcctttggtagcttctagaggaagtttgtg | |
| | | tggaggtaaaatttttgagaccttgcatgtctcatgtttgattgatactttatacgtttaggtaggag | |
| | | gtaattttccttcaggactttaaaaatattgttgctccattttctttgtttctattgttgtattgagaaatc | |
| | | caatgccattttgatttccccatcataaatttcatgatgatgtgtcttggtgtgggtctatatttatcc | |
| | | attgtattgggttttaggtgaacccttccagatagtaactcatttctgtcagttctgggaaacactt | |
| | | agcattggttgatgatttattctctgctgctttgttctcccaactattatttggatgttggatatccag | |
| | | cactgggtatctattttcttacctccctcccttgaccccagtctctgttttttagctctttagctcaatc | |
| | | ttccaactctttgctattgtattttaaaatcttaagaccccttcttgatttgtagaagttccttttcttac | |
| | | aaccaaaaagcctttatctatggatttgttcacagataaggggtattcaatatagtgtatttttttttc | |
| | | atttaaaattgtttgcgcatctatttcctccaaatttctttctgtatttattttttgttgtctatatttcagac | |
| | | ttttccaggatatctgataatctttggctgtcttcttatggttgaaagagggactaaaaagcttgga | |
| | | aagcctttgggttgtgggaaggggctgtctttaggattatctgaatgggcttttttgggagtcccc | |
| | | tcctccacatgaatattttggttttgtcagattccctagaatagaggcttccaatctccttcctgga | |
| | | ggggtctgtccaggaaggagattgtctaggggtctgtcagacagcagctttcagctacttcctt | |
| | | gatctttttcactaatgattatatagtcatctaactactgtcaacaagtaatagatatcctatccttca | |
| | | cttgtttagattatttgctgagataacctctcaaaagaacctctcaaaataaaaggttaacaagag | |
| | | cctatatcttatatttttcttctctttatcttgttagaagatagctattaaaacctgttctttttctgtcttg | |
| | | ataaacacacttcaatcttggtagaatggtagatgggacagtatattttaggacctaaagctctg | |
| | | caaatgtatgatcagcttgtaagtacaggtgctcaaaaacatgtaaacaatcatgctttttactct | |
| | | gtaggaatatctttaaaattcttgtgaatttttccccagaagtaaagcaaatcttcccccagaaata | |
| | | aaattaaatgtgcataatctaaagctttttttttttattgtggtaggatatatatataaaacataatttg | |
| | | ccattgtaaacattttaaatttacaagtcagaggcattaattacatcacaatgttgtgaaattattac | |
| | | tactatttccaaaattttctcatcaccccaaactgaaactctgtaactgttgagcaataacctcattc | |
| | | ctgtatctctcccaaccccaggtaacctcaaatctttctttttatctttgagacaaggtctcattctat | |
| | | cactcaggtaggagtgcagtggtgtgatcatagctcattgcagcctcaaaatcctgggctcaa | |
| | | gcaatcctccttgagtagctaagactataggcacacattaactgcgcctggctgattttgttttttg | |
| | | tagagatgtggtcttgctatgtttcccatgctggtcttgagttcctggcctcaagcagtccttaag | |
| | | attcatccatgttgtggcatgtgtcagaatttcatttgtttttatgactaaataatattccattgtatgt | |
| | | atatacattttgttcatccatcttctgatgaacactgggatatgtctaccttttggctattgtgaataa | |
| | | tgctgcagtaaacattgacataacaagtatgtatttgattgcctgtttctaagttcttttgggtatac | |
| | | atcttgagtagaattgctagataatgtcatgttttatttctcttgtgatttcttcttcgatcccctggttg | |
| | | agtgtgttaatttctacatgtttatgaatttcccactgtttttttgttattgatttccaagttcattccatt | |
| | | gtgattagagaagatacttagtatgattttaatgtttttgagaattggtgtgtggcctgatagatgg | |
| | | tctgtcctggagaatgttcctcatacacttgagcaaaatatttatcatgctattgttgactgtagtttt | |
| | | ctatatgtctcttaggtcaaggtggtttacaatgtgttaaggttctctttttttaaaaaaatttttgcac | |
| | | agagtatctttttctatgtgttccatgtatttgtgtctttggagctatagtctcttgtagacagcatatc | |
| | | actatcttgttttgttttgttttttctgtccattctgccaatttctgccttttgattggaaaatttaatccat | |
| | | ttgcatttaaagtaattaaggaaggactttcttctaccatttaacacttcttctatatgtcatatactttt | |
| | | ttggcccctcatttcctctttatggccttcttttctgtttttttgtagtgaactagtctgattctctttcca | |
| | | ctcccctttgtgtatatttgttagatgttttatttgtggttgctatggggattatagttaacatcctaca | |
| | | cttaaaacaatctaatttaaactgataccaatttaccttcaatagcatacaaaatctctactcctgta | |
| | | aagctctgcccctgccccccttatgttattgatggcacaaattgcctaataaataatttatagttatt | |
| | | tgtatgagtttgtcttttaaatcatttaggaaataaaaagtggagttagaaaacagtatgatagtaa | |
| | | tactgacttttatatttgtcaatatatttatcttattttggatccttatttcattatatagatttgagttact | |
| | | gtctagtgcccttccatttcggcccaaaggattcccttatgcatttcttgcagggcaagtctaatt | |
| | | gtaataaactccctcagcttttgttttatctgagaatgtcttgatttctcccttatttttgatggataatt | |
| | | ttgccagatacatgaatttttggtaacagtatttttctttcagcactttaaatatgtcatcccactacc | |
| | | ttctgacttcatggtttctcatgagatattagatgttataaaatttgaggattcctcattcttgatgag | |
| | | tcagttctgtcttattgcttttcggatttgctcagcttttgtcttttgacagtttgattataacgcggct | |
| | | cagtgtgggtctctgagtttatcccacttagagtttgttgagtttcttggagtcatagatttatgtctt | |
| | | ttatcaaattttggacatatttggctattatttcttcaatttttttcactgcttctttcttttccttctgaaat | |
| | | attcttaatgtatatgttggtctgtttgatgctgtctcaccagtttcttaggctgtgttctcttttgttcct | |
| | | cagacttgattattgcagttgcccttctttttatttttttcaagtttgttgattcttctccctgttcagatc | |
| | | aactgttgaactcctctagtgaatttatttcagttactgtacttttcagctccaagatttatctttggtt | |
| | | cctttttataacgtctgtgtctttattgatattctcattttgttcatatgtctctttcttcctttagttctttgt | |
| | | ccatgttttcctttagctctttgggcttatttaagacaattgtttaaagtctttgcatagtaagtccaat | |
| | | gtctgtgtttcttcagggatggttttcattattttgttttcaatgagccatactttcctgtgtctttgtat | |
| | | gctgtctttttgttgttgaaaactgtatgtttgaacatcataacgtggtggccctgaaaatcagata | |
| | | ttccccccttcctgagagttagttttatttttattattgaagattgtagcagtctattgctacatgtgca | |
| | | gtcatttccaaactatttttgcaaagactgtattccttctgtgtgtcatcactgaagtctctgttcctt | |
| | | agtttgtgtttaatagtttgacatagatttccttgaaaggagttaaaactagcagaaaaatctctct | |
| | | cccagtctttccagtctttgtagattggttctgtgctgggcttttccattaatacttagccaggcttg | |
| | | tactgagcctaacaatcaggcccaaaagcgtagggtctttgcagatcttgtctgagcatgcttct | |
| | | tgctgtgtatgcacgtagttttctaaatctccctgtatgtgctgttgaatattctaatttcccaaaga | |
| | | aactcctttgcagctttttctcacagaacatagatggttttttggatatcttgaccatagtctttcgac | |
| | | ccaggtgtttgcggttgttagttcaccttacacttttttcaagcattgcctactgcttacgatgagtg | |
| | | ctctgtcaatcctttaagtagccccagacaggctaccagagacttaaacaagaatttgtaagttc | |
| | | tgctcagcttcctctagaaatggggatcagggtccaagacagaatgcagttgctgatttcaaga | |
| | | ctgctgcaacaccagggagcttgtgggggaagggcaagcagaaatgtcacaaagctttcttg | |
| | | ccattttaaagttgcctgttcttgactcagcatttgcttcattgctataaactttttactgtttttcagag | |
| | | ttctgataaaattggctatgcctgttcctgctttaaaaaatatatatatattttttagggattggggtc | |
| | | tcactatactgaccaggctggtcttgaacttctggcctcaagccatcctctcatttcagcttccca | |
| | | aagtgctgcaattacacgcgtgaaccaccacacccagcccctgcttgtttttcaatgtgcctact | |
| | | ccaccatgttgctcaagtatgtatattttctaaactaccttgtagtgttgtgatgggaaataaatcc | |
| | | ctgagccttttgaataactcagagagatcaaaaacttagtttatcctattcgaaggattagaaaaa | |
| | | tgatatatctttcactttttcagggataggctcctcattagaaggctcctatgtgccgatgctgtac | |
| | | aagacatttcatttctcttaatgtttacaacaagcttgttgccaaggctgatcttgaactcctggcc | |
| | | tcaaacgatcctcccagctcagtctcacaaagtgttgggatgtctggccaactaatgactatctt | |
| | | aactcttgtgtttcaatgtttatgccttcttttatcttgactgattgtatgactatgtcttctagaacaat | |
| | | gttgaacagaaatggtgagagcagacatccttgctttaatatttcaccattatatatgatgttaggt | |
| | | atagatttttctcacagatgccttttatcagattgaggaatttatattcctactttgccgaaaggttttt | |
| | | gtagtatgagggggtgctgaattttgtcaaacactttttcggtaataattgagatgattggttctgc | |
| | | agtcatcgagatgtggattttctcctttattctgttcgtgagtgattacactggttgactaatgttaa | |
| | | aacaaccttactttccaggaataaaccctattatcttttttataca | |
| PSMC4 | NM_153001.2 | tgcgggtacggacagcgcatgagcttatgttgagggcggagcccagaccagcccttcgtcct | 48 |
| | | atcctgcccttccagcacctctcagccgtaacttaaactacacttcccagaagcctcctcagcc | |
| | | agggacttccgttgtcgtcagcggaagcggtgacagatcatcccaggccacacagaggccg | |
| | | gcttggtcactatggaggagataggcatcttggtggagaaggctcaggatgagatcccagca | |
| | | ctgtccgtgtcccggccccagaccggcctgtccttcctgggccctgagcctgaggacctgga | |
| | | ggacctgtacagccgctacaaggaggaggtgaagcgaatccaaagcatcccgctggtcatc | |
| | | ggacaatttctggaggctgtggatcagaatacagccatcgtgggctctaccacaggctccaac | |
| | | tattatgtgcgcatcctgagcaccatcgatcgggagctgctcaagcccaacgcctcagtggcc | |
| | | ctccacaagcacagcaatgcactggtggacgtgctgccccccgaagccgacagcagcatca | |
| | | tgatgctcacctcagaccagaagccagatgtgatgtacgcggacatcggaggcatggacatc | |
| | | cagaagcaggaggtgcgggaggccgtggagctcccgctcacgcatttcgagctctacaagc | |
| | | agatcggcatcgatcccccccgaggcgtcctcatgtatggcccacctggctgtgggaagacc | |
| | | atgttggcaaaggcggtggcacatcacacaacagctgcattcatccgggtcgtgggctcgga | |
| | | gtttgtacagaagtatctgggtgagggcccccgcatggtccgggatgtgttccgcctggccaa | |
| | | ggagaatgcacctgccatcatcttcatagacgagattgatgccatcgccaccaagagattcga | |
| | | tgctcagacaggggccgacagggaggttcagaggatcctgctggagctgctgaatcagatg | |
| | | gatggatttgatcagaatgtcaatgtcaaggtaatcatggccacaaacagagcagacaccctg | |
| | | gatccggccctgctacggccaggacggctggaccgtaaaattgaatttccacttcctgaccgc | |
| | | cgccagaagagattgattttctccactatcactagcaagatgaacctctctgaggaggttgactt | |
| | | ggaagactatgtggcccggccagataagatttcaggagctgatattaactccatctgtcagga | |
| | | gagtggaatgttggctgtccgtgaaaaccgctacattgtcctggccaaggacttcgagaaagc | |
| | | atacaagactgtcatcaagaaggacgagcaggagcatgagttttacaagtgacccttcccttc | |
| | | cctccaccacaccactcaggggctggggcttctctcgcacccccagcacctctgtcccaaaa | |
| | | cctcattcccttttttctttacccaggattggtttcttcaataaatagataagatcgaatccatttaatt | |
| | | tcttcttagaagtttaactcctttggagaatgtgggccttgaataggatcctctgggtccctcttaa | |
| | | tctgacagatgagcagacgaggtgcatggcctgggttgcagcttgagagaaccaaaatattc | |
| | | aaaccagatgacttccaaaatgtggggaaagggatggaaaatgaacctgagatggagtcctt | |
| | | aatcacgggataaagccctgtgcatctccctcatttcctacaggtaaaagacagtaaagaaatt | |
| | | caggtcacaggccttgggagttcataggaaggagatgtccagtgctgtccagtagaacttt | |
| SF3A1 | NM_005877.5 | ggtcccggaagtgcgccagtcgtaccttcgcggccgcaactcgctcggccgccgccatcttg | 49 |
| | | cgagctcgtcgtactgaccgagcggggaggctgtcttgaggcggcaccgctcaccgacacc | |
| | | gaggcggactggcagccctgagcgtcgcagtcatgccggccggacccgtgcaggcggtg | |
| | | cccccgccgccgcccgtgcccacggagcccaaacagcccacagaagaagaagcatcttca | |
| | | aaggaggattctgcaccttctaagccagttgtggggattatttaccctcctccagaggtcagaa | |
| | | atattgttgacaagactgccagctttgtggccagaaacgggcctgaatttgaagctaggatccg | |
| | | acagaacgagatcaacaaccccaagttcaactttctgaaccccaatgacccttaccatgcctac | |
| | | taccgccacaaggtcagcgagttcaaggaagggaaggctcaggagccgtccgccgccatc | |
| | | cccaaggtcatgcagcagcagcagcagaccacccagcagcagctgccccagaaggtccaa | |
| | | gcccaagtaatccaagagaccatcgtgcccaaagagcctcctcctgagtttgagttcattgctg | |
| | | atcctccctctatctcagccttcgacttggatgtggtgaagctgacggctcagtttgtggccagg | |
| | | aatgggcgccagtttctgacccagctgatgcagaaagagcagcgcaactaccagtttgacttt | |
| | | ctccgcccacagcacagcctcttcaactacttcacgaagctagtggaacagtacaccaagatc | |
| | | ttgattccacccaaaggtttattttcaaagctcaagaaagaggctgaaaacccccgagaagtttt | |
| | | ggatcaggtgtgttaccgagtggaatgggccaaattccaggaacgtgagaggaagaaggaa | |
| | | gaagaggagaaggagaaggagcgggtggcctatgctcagatcgactggcatgattttgtggt | |
| | | ggtggaaacagtggacttccaacccaatgagcaagggaacttccctccccccaccacgcca | |
| | | gaggagctgggggcccgaatcctcattcaggagcgctatgaaaagtttggggagagtgagg | |
| | | aagttgagatggaggtcgagtctgatgaggaggatgacaaacaggagaaggcggaggagc | |
| | | ctccttcccagctggaccaggacacccaagtacaagatatggatgagggttcagatgatgaa | |
| | | gaagaagggcagaaagtgcccccacccccagagacacccatgcctccacctctgccccca | |
| | | actccagaccaagtcattgtccgcaaggattatgatcccaaagcctccaagcccttgcctcca | |
| | | gcccctgctccagatgagtatcttgtgtcccccattactggggagaagatccccgccagcaaa | |
| | | atgcaggaacacatgcgcattggacttcttgaccctcgctggctggagcagcgggatcgctc | |
| | | catccgtgagaagcagagcgatgatgaggtgtacgcaccaggtctggatattgagagcagct | |
| | | tgaagcagttggctgagcggcgtactgacatcttcggtgtagaggaaacagccattggtaag | |
| | | aagatcggtgaggaggagatccagaagccagaggaaaaggtgacctgggatggccactca | |
| | | ggcagcatggcccggacccagcaggctgcccaggccaacatcaccctccaggagcagatt | |
| | | gaggccattcacaaggccaaaggcctggtgccagaggatgacactaaagagaagattggcc | |
| | | ccagcaagcccaatgaaatccctcaacagccaccgccaccatcttcagccaccaacatcccc | |
| | | agctcggctccacccatcacttcagtgccccgaccacccacaatgccacctccagttcgtact | |
| | | acagttgtctccgcagtacccgtcatgccccggcccccaatggcatctgtggtccggctgccc | |
| | | ccaggctcagtgatcgcccccatgccgcccatcatccacgcgcccagaatcaacgtggtgcc | |
| | | catgcctccctcggcccctcctattatggccccccgcccaccccccatgattgtgccaacagc | |
| | | ctttgtgcctgctccacctgtggcacctgtcccagctccagccccaatgccccctgtgcatccc | |
| | | ccacctcccatggaagatgagcccacctccaaaaaactgaagacagaggacagcctcatgc | |
| | | cagaggaggagttcctgcgcagaaacaagggtccagtgtccatcaaagtccaggtgcccaa | |
| | | catgcaggataagacggaatggaaactgaatgggcaggtgctggtcttcaccctcccactca | |
| | | cggaccaggtctctgtcattaaggtgaagattcatgaagccacaggcatgcctgcagggaaa | |
| | | cagaagctacagtatgagggtatcttcatcaaagattccaactcactggcttactacaacatgg | |
| | | ccaatggcgcagtcatccacctggccctcaaggagagaggcgggaggaagaagtagacaa | |
| | | gaggaacctgctgtcaagtccctgccattttgcctctcctgtctcccaccccctgccccagacc | |
| | | caggagcccccctgaggctttgccttgcctgcatatttgtttcgctcttactcagtttgggaattca | |
| | | aattgtcctgcagaggttcattcccctgaccctttccccacattggtaagagtagctgggttttct | |
| | | aagccactctctggaatctctttgtgttagggtctcgatttgaggacattcatttcttcagcagccc | |
| | | attagcaactgagagcccagggatgtcctacaggatagtttcatagtgacaggtggcacttgg | |
| | | ctaatagaatatggctgatattgtcattaatcattttgtaccttgacatgggttgtctaataaaactc | |
| | | ggacccttcttgtgaaatcagttaaataagacttgtctcggtcacctgtgccctgtccagactcg | |
| | | aggcagtggtaacactgcacagtgctatgtggcttctctttgaggatttttgggttttgtaactaaa | |
| | | ttcttgctgccctcatactttttatgtattagaatcatattcgtattgcccttttaaaacattgggatcc | |
| | | tccaaaggcctgccccatgtatttaacagtaatacaggaagcatggcaggcaccatgcaaac | |
| | | caaggatggatggtgcagtccctgtgtcagtgggcggtggtttcctgctggcctggaatcact | |
| | | catcacctgattgattggctctgtggtcctgggcaggtgcctcataggtgtgtggatatgatgac | |
| | | gtttctttaaaatgtatgtatttaacaaatacttaattgtattaaggtcatgtaccaaggatttgataa | |
| | | agtttaaataatttactctctacttttatccattttatccattttaactcatgtaatcctcatgtgagtatt | |
| | | cctgtttaacacttgagtaaactgaggcacagagaacataagttgcatgccatagtcacacact | |
| | | gtgaaagtgaaaagagaatgtgtgcaaaacacgtcacagtcctggtttctgagtaaaggcag | |
| | | gctgttatctttagaatcaagctatcacagggagataggcaatgctgtgggtgttggaggaag | |
| | | gtgagagcctgttgctaacaatttcctggttttaaagctaaggctgattttattgggaagatctca | |
| | | catgtgtgtggcccctgagagttcccagtgccttttatttgcagtccttccatttggacctcctagc | |
| | | tgccccatcaggtcatctccagggctcagaggggtgagaccatttcccaaggtcacagaacc | |
| | | agctctctagtcaccaccctgcctctccctctcacccagagtcagtaccagttttatggctttatta | |
| | | caaactgctgggtccctcccattttcaacttgattgatgggatgtcatcccttatcctgtctgacat | |
| | | ttgcctctggcctggttgctagaagtttgccccaggggcaagagttgaaatttggcttcctgag | |
| | | gtgggctttgtggtttgcgtccctaaagtgagcccactactggttgcttgtccatggccaacacc | |
| | | agaaatcccctgagcactacctgggtctcattccaagaaggaagagggtcaggagacctgg | |
| | | ggagtctcatattccaagttcttctttctttctgggagcagtgggcagttcatggtgttagggcac | |
| | | tcacccccacagactggcaaaccctgcaggacttccgtggctgaggctgtgaccggaggcc | |
| | | aggaatgccgttgggtggattgtgagtgaatgggccctttgagctgccctctagagagcaaat | |
| | | ccagtttcctggagctcctgaatgaatatctgtactggctcgctcagatgcagaagctccattga | |
| | | ccatgaggccttgtgaacatcagtggccacaggcccagtgtgctgcttggcactgcactagttt | |
| | | aggacctgcagcatgtaggtagcgtcctagtgtttataatacaaagctgctctgcacagcttttc | |
| | | tgattcttcttgcaatctcctgaggattatctgccccatttttaaaacgaggtggaatacccaagg | |
| | | tcatgtagccagtgagtgctctggaaagccaaagcagctcatcccttcctggggaccacactg | |
| | | ctctgctccaccagaccacactatgaaataggaataagtgctcctgttgcaggactgctggga | |
| | | aaacaggtggtgtgggacttaagtcaccataattttgaagacttgcatgcagagggctccagg | |
| | | aattgtagacattaaggaatttcactttcagttctacccactacttaagtacttgtcatgtactctta | |
| | | gaggaggccagtaatgatcagaaccattttactttaaaattaataatattgtattagagaatatatt | |
| | | aaatggttatattgggttatgttaggatatatacttgaatggaaatacatgtactattagcaatcata | |
| | | tttcatttatccctgtaattagacaagaaagcataatatagctctactcatgggtacacataccagt | |
| | | gtataagatttttagaagtttactttttaaaaataaaagcaaaatgtaagatcttaaaaaaaaaaaa | |
| | | aaaaaa | |
| PUM1 | NM_001020658.1 | agtgggccgccatgttgtcggagtgaaaggtaagggggagcgagagcgccagagagaga | 50 |
| | | agatcggggggctgaaatccatcttcatcctaccgctccgcccgtgttggtggaatgagcgtt | |
| | | gcatgtgtcttgaagagaaaagcagtgctttggcaggactctttcagcccccacctgaaacatc | |
| | | accctcaagaaccagctaatcccaacatgcctgttgttttgacatctggaacagggtcgcaagc | |
| | | gcagccacaaccagctgcaaatcaggctcttgcagctgggactcactccagccctgtcccag | |
| | | gatctataggagttgcaggccgttcccaggacgacgctatggtggactacttctttcagaggc | |
| | | agcatggtgagcagcttgggggaggaggaagtggaggaggcggctataataatagcaaac | |
| | | atcgatggcctactggggataacattcatgcagaacatcaggtgcgttccatggatgaactga | |
| | | atcatgattttcaagcacttgctctggagggaagagcgatgggagagcagctcttgccaggta | |
| | | aaaagttttgggaaacagatgaatccagcaaagatggaccaaaaggaatattcctgggtgatc | |
| | | aatggcgagacagtgcctggggaacatcagatcattcagtttcccagccaatcatggtgcaga | |
| | | gaagacctggtcagagtttccatgtgaacagtgaggtcaattctgtactgtccccacgatcgga | |
| | | gagtgggggactaggcgttagcatggtggagtatgtgttgagctcatccccgggcgattcctg | |
| | | tctaagaaaaggaggatttggcccaagggatgcagacagtgatgaaaacgacaaaggtgaa | |
| | | aagaagaacaagggtacgtttgatggagataagctaggagatttgaaggaggagggtgatgt | |
| | | gatggacaagaccaatggtttaccagtgcagaatgggattgatgcagacgtcaaagattttag | |
| | | ccgtacccctggtaattgccagaactctgctaatgaagtggatcttctgggtccaaaccagaat | |
| | | ggttctgagggcttagcccagctgaccagcaccaatggtgccaagcctgtggaggatttctcc | |
| | | aacatggagtcccagagtgtccccttggaccccatggaacatgtgggcatggagcctcttca | |
| | | gtttgattattcaggcacgcaggtacctgtggactcagcagcagcaactgtgggactttttgact | |
| | | acaattctcaacaacagctgttccaaagacctaatgcgcttgctgtccagcagttgacagctgc | |
| | | tcagcagcagcagtatgcactggcagctgctcatcagccgcacatcggtttagctcccgctgc | |
| | | gtttgtccccaatccatacatcatcagcgctgctcccccagggacggacccctacacagctgg | |
| | | attggctgcagcagcgacactaggcccagctgtggtccctcaccagtattatggagttactcc | |
| | | ctggggagtctaccctgccagtcttttccagcagcaagctgccgctgccgctgcagcaactaa | |
| | | ttcagctaatcaacagaccaccccacaggctcagcaaggacagcagcaggttctccgtgga | |
| | | ggagccagccaacgtcctttgaccccaaaccagaaccagcagggacagcaaacggatccc | |
| | | cttgtggcagctgcagcagtgaattctgcccttgcatttggacaaggtctggcagcaggcatg | |
| | | ccaggttatccggtgttggctcctgctgcttactatgaccaaactggtgcccttgtagtgaatgc | |
| | | aggcgcgagaaatggtcttggagctcctgttcgacttgtagctcctgccccagtcatcattagtt | |
| | | cctcagctgcacaagcagctgttgcagcagccgcagcttcagcaaatggagcagctggtggt | |
| | | cttgctggaacaacaaatggaccatttcgccctttaggaacacagcagcctcagccccagccc | |
| | | cagcagcagcccaataacaacctggcatccagttctttctacggcaacaactctctgaacagc | |
| | | aattcacagagcagctccctcttctcccagggctctgcccagcctgccaacacatccttgggat | |
| | | tcggaagtagcagttctctcggcgccaccctgggatccgcccttggagggtttggaacagca | |
| | | gttgcaaactccaacactggcagtggctcccgccgtgactccctgactggcagcagtgacctt | |
| | | tataagaggacatcgagcagcttgacccccattggacacagtttttataacggccttagcttttc | |
| | | ctcctctcctggacccgtgggcatgcctctccctagtcagggaccaggacattcacagacacc | |
| | | acctccttccctctcttcacatggatcctcttcaagcttaaacctgggaggactcacgaatggca | |
| | | gtggaagatacatctctgctgctccaggcgctgaagccaagtaccgcagtgcaagcagcgc | |
| | | ctccagcctcttcagcccgagcagcactcttttctcttcctctcgtttgcgatatggaatgtctgat | |
| | | gtcatgccttctggcaggagcaggcttttggaagattttcgaaacaaccggtaccccaatttac | |
| | | aactgcgggagattgctggacatataatggaattttcccaagaccagcatgggtccagattcat | |
| | | tcagctgaaactggagcgtgccacaccagctgagcgccagcttgtcttcaatgaaatcctcca | |
| | | ggctgcctaccaactcatggtggatgtgtttggtaattacgtcattcagaagttctttgaatttggc | |
| | | agtcttgaacagaagctggctttggcagaacggattcgaggccacgtcctgtcattggcacta | |
| | | cagatgtatggctgccgtgttatccagaaagctcttgagtttattccttcagaccagcaggtaatt | |
| | | aatgagatggttcgggaactagatggccatgtcttgaagtgtgtgaaagatcagaatggcaat | |
| | | cacgtggttcagaaatgcattgaatgtgtacagccccagtctttgcaatttatcatcgatgcgttt | |
| | | aagggacaggtatttgccttatccacacatccttatggctgccgagtgattcagagaatcctgg | |
| | | agcactgtctccctgaccagacactccctattttagaggagcttcaccagcacacagagcagc | |
| | | ttgtacaggatcaatatggaaattatgtaatccaacatgtactggagcacggtcgtcctgaggat | |
| | | aaaagcaaaattgtagcagaaatccgaggcaatgtacttgtattgagtcagcacaaatttgcaa | |
| | | gcaatgttgtggagaagtgtgttactcacgcctcacgtacggagcgcgctgtgctcatcgatg | |
| | | aggtgtgcaccatgaacgacggtccccacagtgccttatacaccatgatgaaggaccagtat | |
| | | gccaactacgtggtccagaagatgattgacgtggcggagccaggccagcggaagatcgtca | |
| | | tgcataagatccggccccacatcgcaactcttcgtaagtacacctatggcaagcacattctggc | |
| | | caagctggagaagtactacatgaagaacggtgttgacttagggcccatctgtggcccccctaa | |
| | | tggtatcatctgaggcagtgtcacccgctgttccctcattcccgctgacctcactggcccactg | |
| | | gcaaatccaaccagcaaccagaaatgttctagtgtagagtctgagacgggcaagtggttgctc | |
| | | caggattactccctcctccaaaaaaggaatcaaatccacgagtggaaaagcctttgtaaattta | |
| | | attttattacacataacatgtactattttttttaattgactaattgccctgctgttttactggtgtatagg | |
| | | atacttgtacataggtaaccaatgtacatgggaggccacatattttgttcactgttgtatctatattt | |
| | | cacatgtggaaactttcagggtggttggtttaacaaaaaaaaaaagctttaaaaaaaaaagaaa | |
| | | aaaaggaaaaggtttttagctcatttgcctggccggcaagttttgcaaatagctcttccccacct | |
| | | cctcattttagtaaaaaacaaacaaaaacaaaaaaacctgagaagtttgaattgtagttaaatga | |
| | | ccccaaactggcatttaacactgtttataaaaaatatatatatatatatatatatatataatgaaaaa | |
| | | ggtttcagagttgctaaagcttcagtttgtgacattaagtttatgaaattctaaaaaatgccttttttg | |
| | | gagactatattatgctgaagaaggctgttcgtgaggaggagatgcgagcacccagaacgtctt | |
| | | ttgaggctgggcgggtgtgattgtttactgcctactggatttttttctattaacattgaaaggtaaa | |
| | | atctgattatttagcatgagaaaaaaaaatccaactctgcttttggtcttgcttctataaatatatag | |
| | | tgtatacttggtgtagactttgcatatatacaaatttgtagtattttcttgttttgatgtctaatctgtat | |
| | | ctataatgtaccctagtagtcgaacatacttttgattgtacaattgtacatttgtatacctgtaatgta | |
| | | aatgtggagaagtttgaatcaacataaacacgttttttggtaagaaaagagaattagccagccc | |
| | | tgtgcattcagtgtatattctcaccttttatggtcgtagcatatagtgttgtatattgtaaattgtaattt | |
| | | caaccagaagtaaatttttttcttttgaaggaataaatgttctttatacagcctagttaatgtttaaaa | |
| | | agaaaaaaatagcttggttttatttgtcatctagtctcaagtatagcgagattctttctaaatgttatt | |
| | | caagattgagttctcactagtgtttttttaatcctaaaaaagtaatgttttgattttgtgacagtcaaa | |
| | | aggacgtgcaaaagtctagccttgcccgagctttccttacaatcagagcccctctcaccttgta | |
| | | aagtgtgaatcgcccttcccttttgtacagaagatgaactgtattttgcattttgtctacttgtaagt | |
| | | gaatgtaacatactgtcaattttccttgtttgaatatagaattgtaacactacacggtgtacatttcc | |
| | | agagccttgtgtatatttccaatgaacttttttgcaagcacacttgtaaccatatgtgtataattaac | |
| | | aaacctgtgtatgcttatgcctgggcaactattttttgtaactcttgtgtagattgtctctaaacaat | |
| | | gtgtgatctttattttgaaaaatacagaactttggaatctgaaaaaaaaaaaaaaaaaaaaaaaa | |
| | | aaaaaaa | |
| ACTB | NM_001101.4 | gagtgagcggcgcggggccaatcagcgtgcgccgttccgaaagttgccttttatggctcgag | 51 |
| | | cggccgcggcggcgccctataaaacccagcggcgcgacgcgccaccaccgccgagacc | |
| | | gcgtccgccccgcgagcacagagcctcgcctttgccgatccgccgcccgtccacacccgcc | |
| | | gccagctcaccatggatgatgatatcgccgcgctcgtcgtcgacaacggctccggcatgtgc | |
| | | aaggccggcttcgcgggcgacgatgccccccgggccgtcttcccctccatcgtggggcgcc | |
| | | ccaggcaccagggcgtgatggtgggcatgggtcagaaggattcctatgtgggcgacgagg | |
| | | cccagagcaagagaggcatcctcaccctgaagtaccccatcgagcacggcatcgtcaccaa | |
| | | ctgggacgacatggagaaaatctggcaccacaccttctacaatgagctgcgtgtggctcccg | |
| | | aggagcaccccgtgctgctgaccgaggcccccctgaaccccaaggccaaccgcgagaag | |
| | | atgacccagatcatgtttgagaccttcaacaccccagccatgtacgttgctatccaggctgtgct | |
| | | atccctgtacgcctctggccgtaccactggcatcgtgatggactccggtgacggggtcaccc | |
| | | acactgtgcccatctacgaggggtatgccctcccccatgccatcctgcgtctggacctggctg | |
| | | gccgggacctgactgactacctcatgaagatcctcaccgagcgcggctacagcttcaccacc | |
| | | acggccgagcgggaaatcgtgcgtgacattaaggagaagctgtgctacgtcgccctggactt | |
| | | cgagcaagagatggccacggctgcttccagctcctccctggagaagagctacgagctgcct | |
| | | gacggccaggtcatcaccattggcaatgagcggttccgctgccctgaggcactcttccagcct | |
| | | tccttcctgggcatggagtcctgtggcatccacgaaactaccttcaactccatcatgaagtgtg | |
| | | acgtggacatccgcaaagacctgtacgccaacacagtgctgtctggcggcaccaccatgtac | |
| | | cctggcattgccgacaggatgcagaaggagatcactgccctggcacccagcacaatgaaga | |
| | | tcaagatcattgctcctcctgagcgcaagtactccgtgtggatcggcggctccatcctggcctc | |
| | | gctgtccaccttccagcagatgtggatcagcaagcaggagtatgacgagtccggcccctcca | |
| | | tcgtccaccgcaaatgcttctaggcggactatgacttagttgcgttacaccctttcttgacaaaac | |
| | | ctaacttgcgcagaaaacaagatgagattggcatggctttatttgttttttttgttttgttttggtttttt | |
| | | ttttttttttggcttgactcaggatttaaaaactggaacggtgaaggtgacagcagtcggttggag | |
| | | cgagcatcccccaaagttcacaatgtggccgaggactttgattgcacattgttgtttttttaatagt | |
| | | cattccaaatatgagatgcgttgttacaggaagtcccttgccatcctaaaagccaccccacttct | |
| | | ctctaaggagaatggcccagtcctctcccaagtccacacaggggaggtgatagcattgctttc | |
| | | gtgtaaattatgtaatgcaaaatttttttaatcttcgccttaatacttttttattttgttttattttgaatgat | |
| | | gagccttcgtgcccccccttcccccttttttgtcccccaacttgagatgtatgaaggcttttggtct | |
| | | ccctgggagtgggtggaggcagccagggcttacctgtacactgacttgagaccagttgaata | |
| | | aaagtgcacaccttaaaaatgaggaaaaaaaaaaaaaaaaaa | |
| GAPD | NM_002046.6 | gctctctgctcctcctgttcgacagtcagccgcatcttcttttgcgtcgccagccgagccacatc | 52 |
| | | gctcagacaccatggggaaggtgaaggtcggagtcaacggatttggtcgtattgggcgcctg | |
| | | gtcaccagggctgcttttaactctggtaaagtggatattgttgccatcaatgaccccttcattgac | |
| | | ctcaactacatggtttacatgttccaatatgattccacccatggcaaattccatggcaccgtcaa | |
| | | ggctgagaacgggaagcttgtcatcaatggaaatcccatcaccatcttccaggagcgagatc | |
| | | cctccaaaatcaagtggggcgatgctggcgctgagtacgtcgtggagtccactggcgtcttca | |
| | | ccaccatggagaaggctggggctcatttgcaggggggagccaaaagggtcatcatctctgc | |
| | | cccctctgctgatgcccccatgttcgtcatgggtgtgaaccatgagaagtatgacaacagcctc | |
| | | aagatcatcagcaatgcctcctgcaccaccaactgcttagcacccctggccaaggtcatccat | |
| | | gacaactttggtatcgtggaaggactcatgaccacagtccatgccatcactgccacccagaag | |
| | | actgtggatggcccctccgggaaactgtggcgtgatggccgcggggctctccagaacatcat | |
| | | ccctgcctctactggcgctgccaaggctgtgggcaaggtcatccctgagctgaacgggaag | |
| | | ctcactggcatggccttccgtgtccccactgccaacgtgtcagtggtggacctgacctgccgt | |
| | | ctagaaaaacctgccaaatatgatgacatcaagaaggtggtgaagcaggcgtcggagggcc | |
| | | ccctcaagggcatcctgggctacactgagcaccaggtggtctcctctgacttcaacagcgaca | |
| | | cccactcctccacctttgacgctggggctggcattgccctcaacgaccactttgtcaagctcatt | |
| | | tcctggtatgacaacgaatttggctacagcaacagggtggtggacctcatggcccacatggc | |
| | | ctccaaggagtaagacccctggaccaccagccccagcaagagcacaagaggaagagaga | |
| | | gaccctcactgctggggagtccctgccacactcagtcccccaccacactgaatctcccctcct | |
| | | cacagttgccatgtagaccccttgaagaggggaggggcctagggagccgcaccttgtcatgt | |
| | | accatcaataaagtaccctgtgctcaaccagttaaaaaaaaaaaaaaaaaaaaa | |
| GUSB | NM_000181.3 | gtcctcaaccaagatggcgcggatggcttcaggcgcatcacgacaccggcgcgtcacgcga | 53 |
| | | cccgccctacgggcacctcccgcgcttttcttagcgccgcagacggtggccgagcggggga | |
| | | ccgggaagcatggcccgggggtcggcggttgcctgggcggcgctcgggccgttgttgtgg | |
| | | ggctgcgcgctggggctgcagggcgggatgctgtacccccaggagagcccgtcgcggga | |
| | | gtgcaaggagctggacggcctctggagcttccgcgccgacttctctgacaaccgacgccgg | |
| | | ggcttcgaggagcagtggtaccggcggccgctgtgggagtcaggccccaccgtggacatg | |
| | | ccagttccctccagcttcaatgacatcagccaggactggcgtctgcggcattttgtcggctggg | |
| | | tgtggtacgaacgggaggtgatcctgccggagcgatggacccaggacctgcgcacaagag | |
| | | tggtgctgaggattggcagtgcccattcctatgccatcgtgtgggtgaatggggtcgacacgc | |
| | | tagagcatgaggggggctacctccccttcgaggccgacatcagcaacctggtccaggtggg | |
| | | gcccctgccctcccggctccgaatcactatcgccatcaacaacacactcacccccaccaccct | |
| | | gccaccagggaccatccaatacctgactgacacctccaagtatcccaagggttactttgtcca | |
| | | gaacacatattttgactttttcaactacgctggactgcagcggtctgtacttctgtacacgacacc | |
| | | caccacctacatcgatgacatcaccgtcaccaccagcgtggagcaagacagtgggctggtg | |
| | | aattaccagatctctgtcaagggcagtaacctgttcaagttggaagtgcgtcttttggatgcaga | |
| | | aaacaaagtcgtggcgaatgggactgggacccagggccaacttaaggtgccaggtgtcagc | |
| | | ctctggtggccgtacctgatgcacgaacgccctgcctatctgtattcattggaggtgcagctga | |
| | | ctgcacagacgtcactggggcctgtgtctgacttctacacactccctgtggggatccgcactgt | |
| | | ggctgtcaccaagagccagttcctcatcaatgggaaacctttctatttccacggtgtcaacaag | |
| | | catgaggatgcggacatccgagggaagggcttcgactggccgctgctggtgaaggacttca | |
| | | acctgcttcgctggcttggtgccaacgctttccgtaccagccactacccctatgcagaggaagt | |
| | | gatgcagatgtgtgaccgctatgggattgtggtcatcgatgagtgtcccggcgtgggcctggc | |
| | | gctgccgcagttcttcaacaacgtttctctgcatcaccacatgcaggtgatggaagaagtggtg | |
| | | cgtagggacaagaaccaccccgcggtcgtgatgtggtctgtggccaacgagcctgcgtccc | |
| | | acctagaatctgctggctactacttgaagatggtgatcgctcacaccaaatccttggacccctc | |
| | | ccggcctgtgacctttgtgagcaactctaactatgcagcagacaagggggctccgtatgtgga | |
| | | tgtgatctgtttgaacagctactactcttggtatcacgactacgggcacctggagttgattcagct | |
| | | gcagctggccacccagtttgagaactggtataagaagtatcagaagcccattattcagagcga | |
| | | gtatggagcagaaacgattgcagggtttcaccaggatccacctctgatgttcactgaagagta | |
| | | ccagaaaagtctgctagagcagtaccatctgggtctggatcaaaaacgcagaaaatacgtgg | |
| | | ttggagagctcatttggaattttgccgatttcatgactgaacagtcaccgacgagagtgctggg | |
| | | gaataaaaaggggatcttcactcggcagagacaaccaaaaagtgcagcgttccttttgcgag | |
| | | agagatactggaagattgccaatgaaaccaggtatccccactcagtagccaagtcacaatgttt | |
| | | ggaaaacagcctgtttacttgagcaagactgataccacctgcgtgtcccttcctccccgagtca | |
| | | gggcgacttccacagcagcagaacaagtgcctcctggactgttcacggcagaccagaacgtt | |
| | | tctggcctgggttttgtggtcatctattctagcagggaacactaaaggtggaaataaaagattttc | |
| | | tattatggaaataaagagttggcatgaaagtggctactgaaaaaaaaaaaaaaaaaaaaaaaaa | |
| RPLP0 | NM_001002.3 | gtctgacgggcgatggcgcagccaatagacaggagcgctatccgcggtttctgattggctac | 54 |
| | | tttgttcgcattataaaaggcacgcgcgggcgcgaggcccttctctcgccaggcgtcctcgtg | |
| | | gaagtgacatcgtctttaaaccctgcgtggcaatccctgacgcaccgccgtgatgcccaggg | |
| | | aagacagggcgacctggaagtccaactacttccttaagatcatccaactattggatgattatcc | |
| | | gaaatgtttcattgtgggagcagacaatgtgggctccaagcagatgcagcagatccgcatgtc | |
| | | ccttcgcgggaaggctgtggtgctgatgggcaagaacaccatgatgcgcaaggccatccga | |
| | | gggcacctggaaaacaacccagctctggagaaactgctgcctcatatccgggggaatgtgg | |
| | | gctttgtgttcaccaaggaggacctcactgagatcagggacatgttgctggccaataaggtgc | |
| | | cagctgctgcccgtgctggtgccattgccccatgtgaagtcactgtgccagcccagaacactg | |
| | | gtctcgggcccgagaagacctcctttttccaggctttaggtatcaccactaaaatctccagggg | |
| | | caccattgaaatcctgagtgatgtgcagctgatcaagactggagacaaagtgggagccagcg | |
| | | aagccacgctgctgaacatgctcaacatctcccccttctcctttgggctggtcatccagcaggt | |
| | | gttcgacaatggcagcatctacaaccctgaagtgcttgatatcacagaggaaactctgcattct | |
| | | cgcttcctggagggtgtccgcaatgttgccagtgtctgtctgcagattggctacccaactgttgc | |
| | | atcagtaccccattctatcatcaacgggtacaaacgagtcctggccttgtctgtggagacggat | |
| | | tacaccttcccacttgctgaaaaggtcaaggccttcttggctgatccatctgcctttgtggctgct | |
| | | gcccctgtggctgctgccaccacagctgctcctgctgctgctgcagccccagctaaggttgaa | |
| | | gccaaggaagagtcggaggagtcggacgaggatatgggatttggtctctttgactaatcacc | |
| | | aaaaagcaaccaacttagccagttttatttgcaaaacaaggaaataaaggcttacttctttaaaa | |
| | | agtaaaaaaaaaaaaaaaaaaaaaaaaa | |
| TFRC | NM_003234.3 | agagcgtcgggatatcgggtggcggctcgggacggaggacgcgctagtgtgagtgcggg | 55 |
| | | cttctagaactacaccgaccctcgtgtcctcccttcatcctgcggggctggctggagcggccg | |
| | | ctccggtgctgtccagcagccatagggagccgcacggggagcgggaaagcggtcgcggc | |
| | | cccaggcggggcggccgggatggagcggggccgcgagcctgtggggaaggggctgtgg | |
| | | cggcgcctcgagcggctgcaggttcttctgtgtggcagttcagaatgatggatcaagctagat | |
| | | cagcattctctaacttgtttggtggagaaccattgtcatatacccggttcagcctggctcggcaa | |
| | | gtagatggcgataacagtcatgtggagatgaaacttgctgtagatgaagaagaaaatgctgac | |
| | | aataacacaaaggccaatgtcacaaaaccaaaaaggtgtagtggaagtatctgctatgggact | |
| | | attgctgtgatcgtctttttcttgattggatttatgattggctacttgggctattgtaaaggggtaga | |
| | | accaaaaactgagtgtgagagactggcaggaaccgagtctccagtgagggaggagccagg | |
| | | agaggacttccctgcagcacgtcgcttatattgggatgacctgaagagaaagttgtcggagaa | |
| | | actggacagcacagacttcaccggcaccatcaagctgctgaatgaaaattcatatgtccctcgt | |
| | | gaggctggatctcaaaaagatgaaaatcttgcgttgtatgttgaaaatcaatttcgtgaatttaaa | |
| | | ctcagcaaagtctggcgtgatcaacattttgttaagattcaggtcaaagacagcgctcaaaact | |
| | | cggtgatcatagttgataagaacggtagacttgtttacctggtggagaatcctgggggttatgtg | |
| | | gcgtatagtaaggctgcaacagttactggtaaactggtccatgctaattttggtactaaaaaaga | |
| | | ttttgaggatttatacactcctgtgaatggatctatagtgattgtcagagcagggaaaatcaccttt | |
| | | gcagaaaaggttgcaaatgctgaaagcttaaatgcaattggtgtgttgatatacatggaccaga | |
| | | ctaaatttcccattgttaacgcagaactttcattctttggacatgctcatctggggacaggtgacc | |
| | | cttacacacctggattcccttccttcaatcacactcagtttccaccatctcggtcatcaggattgc | |
| | | ctaatatacctgtccagacaatctccagagctgctgcagaaaagctgtttgggaatatggaag | |
| | | gagactgtccctctgactggaaaacagactctacatgtaggatggtaacctcagaaagcaaga | |
| | | atgtgaagctcactgtgagcaatgtgctgaaagagataaaaattcttaacatctttggagttatta | |
| | | aaggctttgtagaaccagatcactatgttgtagttggggcccagagagatgcatggggccctg | |
| | | gagctgcaaaatccggtgtaggcacagctctcctattgaaacttgcccagatgttctcagatat | |
| | | ggtcttaaaagatgggtttcagcccagcagaagcattatctttgccagttggagtgctggagac | |
| | | tttggatcggttggtgccactgaatggctagagggatacctttcgtccctgcatttaaaggctttc | |
| | | acttatattaatctggataaagcggttcttggtaccagcaacttcaaggtttctgccagcccactg | |
| | | ttgtatacgcttattgagaaaacaatgcaaaatgtgaagcatccggttactgggcaatttctatat | |
| | | caggacagcaactgggccagcaaagttgagaaactcactttagacaatgctgctttccctttcc | |
| | | ttgcatattctggaatcccagcagtttctttctgtttttgcgaggacacagattatccttatttgggta | |
| | | ccaccatggacacctataaggaactgattgagaggattcctgagttgaacaaagtggcacga | |
| | | gcagctgcagaggtcgctggtcagttcgtgattaaactaacccatgatgttgaattgaacctgg | |
| | | actatgagaggtacaacagccaactgctttcatttgtgagggatctgaaccaatacagagcag | |
| | | acataaaggaaatgggcctgagtttacagtggctgtattctgctcgtggagacttcttccgtgct | |
| | | acttccagactaacaacagatttcgggaatgctgagaaaacagacagatttgtcatgaagaaa | |
| | | ctcaatgatcgtgtcatgagagtggagtatcacttcctctctccctacgtatctccaaaagagtct | |
| | | cctttccgacatgtcttctggggctccggctctcacacgctgccagctttactggagaacttgaa | |
| | | actgcgtaaacaaaataacggtgcttttaatgaaacgctgttcagaaaccagttggctctagcta | |
| | | cttggactattcagggagctgcaaatgccctctctggtgacgtttgggacattgacaatgagttt | |
| | | taaatgtgatacccatagcttccatgagaacagcagggtagtctggtttctagacttgtgctgat | |
| | | cgtgctaaattttcagtagggctacaaaacctgatgttaaaattccatcccatcatcttggtactac | |
| | | tagatgtctttaggcagcagcttttaatacagggtagataacctgtacttcaagttaaagtgaata | |
| | | accacttaaaaaatgtccatgatggaatattcccctatctctagaattttaagtgctttgtaatggg | |
| | | aactgcctctttcctgttgttgttaatgaaaatgtcagaaaccagttatgtgaatgatctctctgaat | |
| | | cctaagggctggtctctgctgaaggttgtaagtggtcgcttactttgagtgatcctccaacttcat | |
| | | ttgatgctaaataggagataccaggttgaaagaccttctccaaatgagatctaagcctttccata | |
| | | aggaatgtagctggtttcctcattcctgaaagaaacagttaactttcagaagagatgggcttgttt | |
| | | tcttgccaatgaggtctgaaatggaggtccttctgctggataaaatgaggttcaactgttgattg | |
| | | caggaataaggccttaatatgttaacctcagtgtcatttatgaaaagaggggaccagaagcca | |
| | | aagacttagtatattttcttttcctctgtcccttcccccataagcctccatttagttctttgttatttttgt | |
| | | ttcttccaaagcacattgaaagagaaccagtttcaggtgtttagttgcagactcagtttgtcagac | |
| | | tttaaagaataatatgctgccaaattttggccaaagtgttaatcttaggggagagctttctgtcctt | |
| | | ttggcactgagatatttattgtttatttatcagtgacagagttcactataaatggtgtttttttaataga | |
| | | atataattatcggaagcagtgccttccataattatgacagttatactgtcggttttttttaaataaaa | |
| | | gcagcatctgctaataaaacccaacagatactggaagttttgcatttatggtcaacacttaaggg | |
| | | ttttagaaaacagccgtcagccaaatgtaattgaataaagttgaagctaagatttagagatgaat | |
| | | taaatttaattaggggttgctaagaagcgagcactgaccagataagaatgctggttttcctaaat | |
| | | gcagtgaattgtgaccaagttataaatcaatgtcacttaaaggctgtggtagtactcctgcaaaa | |
| | | ttttatagctcagtttatccaaggtgtaactctaattcccattttgcaaaatttccagtacctttgtca | |
| | | caatcctaacacattatcgggagcagtgtcttccataatgtataaagaacaaggtagtttttacct | |
| | | accacagtgtctgtatcggagacagtgatctccatatgttacactaagggtgtaagtaattatcg | |
| | | ggaacagtgtttcccataattttcttcatgcaatgacatcttcaaagcttgaagatcgttagtatct | |
| | | aacatgtatcccaactcctataattccctatcttttagttttagttgcagaaacattttgtggtcatta | |
| | | agcattgggtgggtaaattcaaccactgtaaaatgaaattactacaaaatttgaaatttagcttgg | |
| | | gtttttgttacctttatggtttctccaggtcctctacttaatgagatagtagcatacatttataatgttt | |
| | | gctattgacaagtcattttaactttatcacattatttgcatgttacctcctataaacttagtgcggac | |
| | | aagttttaatccagaattgaccttttgacttaaagcagagggactttgtatagaaggtttggggg | |
| | | ctgtggggaaggagagtcccctgaaggtctgacacgtctgcctacccattcgtggtgatcaat | |
| | | taaatgtaggtatgaataagttcgaagctccgtgagtgaaccatcattataaacgtgatgatcag | |
| | | ctgtttgtcatagggcagttggaaacggcctcctagggaaaagttcatagggtctcttcaggttc | |
| | | ttagtgtcacttacctagatttacagcctcacttgaatgtgtcactactcacagtctctttaatcttca | |
| | | gttttatctttaatctcctcttttatcttggactgacatttagcgtagctaagtgaaaaggtcatagct | |
| | | gagattcctggttcgggtgttacgcacacgtacttaaatgaaagcatgtggcatgttcatcgtat | |
| | | aacacaatatgaatacagggcatgcattttgcagcagtgagtctcttcagaaaacccttttctac | |
| | | agttagggttgagttacttcctatcaagccagtacgtgctaacaggctcaatattcctgaatgaa | |
| | | atatcagactagtgacaagctcctggtcttgagatgtcttctcgttaaggagatgggccttttgg | |
| | | aggtaaaggataaaatgaatgagttctgtcatgattcactattctagaacttgcatgacctttact | |
| | | gtgttagctctttgaatgttcttgaaattttagactttctttgtaaacaaatgatatgtccttatcattgt | |
| | | ataaaagctgttatgtgcaacagtgtggagattccttgtctgatttaataaaatacttaaacactga | |
| | | aaaaaaaaaa | |
| 18S | X03205.1 | tacctggttgatcctgccagtagcatatgcttgtctcaaagattaagccatgcatgtctaagtacg | 56 |
| | | cacggccggtacagtgaaactgcgaatggctcattaaatcagttatggttcctttggtcgctcg | |
| | | ctcctctcccacttggataactgtggtaattctagagctaatacatgccgacgggcgctgaccc | |
| | | ccttcgcgggggggatgcgtgcatttatcagatcaaaaccaacccggtcagcccctctccgg | |
| | | ccccggccggggggcgggcgccggcggctttggtgactctagataacctcgggccgatcg | |
| | | cacgccccccgtggcggcgacgacccattcgaacgtctgccctatcaactttcgatggtagtc | |
| | | gccgtgcctaccatggtgaccacgggtgacggggaatcagggttcgattccggagaggga | |
| | | gcctgagaaacggctaccacatccaaggaaggcagcaggcgcgcaaattacccactcccg | |
| | | acccggggaggtagtgacgaaaaataacaatacaggactctttcgaggccctgtaattggaat | |
| | | gagtccactttaaatcctttaacgaggatccattggagggcaagtctggtgccagcagccgcg | |
| | | gtaattccagctccaatagcgtatattaaagttgctgcagttaaaaagctcgtagttggatcttgg | |
| | | gagcgggcgggcggtccgccgcgaggcgagccaccgcccgtccccgccccttgcctctc | |
| | | ggcgccccctcgatgctcttagctgagtgtcccgcggggcccgaagcgtttactttgaaaaaa | |
| | | ttagagtgttcaaagcaggcccgagccgcctggataccgcagctaggaataatggaatagga | |
| | | ccgcggttctattttgttggttttcggaactgaggccatgattaagagggacggccgggggcat | |
| | | tcgtattgcgccgctagaggtgaaattcttggaccggcgcaagacggaccagagcgaaagc | |
| | | atttgccaagaatgttttcattaatcaagaacgaaagtcggaggttcgaagacgatcagatacc | |
| | | gtcgtagttccgaccataaacgatgccgaccggcgatgcggcggcgttattcccatgacccg | |
| | | ccgggcagcttccgggaaaccaaagtctttgggttccggggggagtatggttgcaaagctga | |
| | | aacttaaaggaattgacggaagggcaccaccaggagtggagcctgcggcttaatttgactca | |
| | | acacgggaaacctcacccggcccggacacggacaggattgacagattgatagctctttctcg | |
| | | attccgtgggtggtggtgcatggccgttcttagttggtggagcgatttgtctggttaattccgata | |
| | | acgaacgagactctggcatgctaactagttacgcgacccccgagcggtcggcgtcccccaa | |
| | | cttcttagagggacaagtggcgttcagccacccgagattgagcaataacaggtctgtgatgcc | |
| | | cttagatgtccggggctgcacgcgcgctacactgactggctcagcgtgtgcctaccctacgc | |
| | | cggcaggcgcgggtaacccgttgaaccccattcgtgatggggatcggggattgcaattattc | |
| | | cccatgaacgaggaattcccagtaagtgcgggtcataagcttgcgttgattaagtccctgccct | |
| | | ttgtacacaccgcccgtcgctactaccgattggatggtttagtgaggccctcggatcggcccc | |
| | | gccggggtcggcccacggccctggcggagcgctgagaagacggtcgaacttgactatcta | |
| | | gaggaagtaaaagtcgtaacaaggtttccgtaggtgaacctgcggaaggatcatta | |
| PPIA | NM_021130.4 | ggggccgaacgtggtataaaaggggcgggaggccaggctcgtgccgttttgcagacgcca | 57 |
| | | ccgccgaggaaaaccgtgtactattagccatggtcaaccccaccgtgttcttcgacattgccgt | |
| | | cgacggcgagcccttgggccgcgtctcctttgagctgtttgcagacaaggtcccaaagacag | |
| | | cagaaaattttcgtgctctgagcactggagagaaaggatttggttataagggttcctgctttcac | |
| | | agaattattccagggtttatgtgtcagggtggtgacttcacacgccataatggcactggtggca | |
| | | agtccatctatggggagaaatttgaagatgagaacttcatcctaaagcatacgggtcctggcat | |
| | | cttgtccatggcaaatgctggacccaacacaaatggttcccagtttttcatctgcactgccaaga | |
| | | ctgagtggttggatggcaagcatgtggtgtttggcaaagtgaaagaaggcatgaatattgtgg | |
| | | aggccatggagcgctttgggtccaggaatggcaagaccagcaagaagatcaccattgctga | |
| | | ctgtggacaactcgaataagtttgacttgtgttttatcttaaccaccagatcattccttctgtagctc | |
| | | aggagagcacccctccaccccatttgctcgcagtatcctagaatctttgtgctctcgctgcagtt | |
| | | ccctttgggttccatgttttccttgttccctcccatgcctagctggattgcagagttaagtttatgatt | |
| | | atgaaataaaaactaaataacaattgtcctcgtttgagttaagagtgttgatgtaggctttattttaa | |
| | | gcagtaatgggttacttctgaaacatcacttgtttgcttaattctacacagtacttagattttttttact | |
| | | ttccagtcccaggaagtgtcaatgtttgttgagtggaatattgaaaatgtaggcagcaactggg | |
| | | catggtggctcactgtctgtaatgtattacctgaggcagaagaccacctgagggtaggagtca | |
| | | agatcagcctgggcaacatagtgagacgctgtctctacaaaaaataattagcctggcctggtg | |
| | | gtgcatgcctagtcctagctgatctggaggctgacgtgggaggattgcttgagcctagagtga | |
| | | gctattatcatgccactgtacagcctgggtgttcacagatcttgtgtctcaaaggtaggcagag | |
| | | gcaggaaaagcaaggagccagaattaagaggttgggtcagtctgcagtgagttcatgcattta | |
| | | gaggtgttcttcaagatgactaatgtcaaaaattgagacatctgttgcggttttttttttttttttttccc | |
| | | ctggaatgcagtggcgtgatctcagctcactgcagcctccgcctcctgggttcaagtgattcta | |
| | | gtgcctcagcctcctgagtagctgggataatgggcgtgtgccaccatgcccagctaatttttgt | |
| | | atttttagtatagatggggtttcatcattttgaccaggctggtctcaaactcttgacctcagctgat | |
| | | gcgcctgccttggcctcccaaactgctgagattacagatgtgagccaccgcaccctacctcatt | |
| | | ttctgtaacaaagctaagcttgaacactgttgatgttcttgagggaagcatattgggctttaggct | |
| | | gtaggtcaagtttatacatcttaattatggtggaattcctatgtagagtctaaaaagccaggtactt | |
| | | ggtgctacagtcagtctccctgcagagggttaaggcgcagactacctgcagtgaggaggtac | |
| | | tgcttgtagcatatagagcctctccctagctttggttatggaggctttgaggttttgcaaacctga | |
| | | ccaatttaagccataagatctggtcaaagggatacccttcccactaaggacttggtttctcagga | |
| | | aattatatgtacagtgcttgctggcagttagatgtcaggacaatctaagctgagaaaaccccttc | |
| | | tctgcccaccttaacagacctctagggttcttaacccagcaatcaagtttgcctatcctagaggt | |
| | | ggcggatttgatcatttggtgtgttgggcaatttttgttttactgtctggttccttctgcgtgaattac | |
| | | caccaccaccacttgtgcatctcagtcttgtgtgttgtctggttacgtattccctgggtgataccat | |
| | | tcaatgtcttaatgtacttgtggctcagacctgagtgcaaggtggaaataaacatcaaacatcttt | |
| | | tcattatcccta | |
| PGK1 | NM_000291.3 | gagagcagcggccgggaaggggcggtgcgggaggcggggtgtggggcggtagtgtgg | 58 |
| | | gccctgttcctgcccgcgcggtgttccgcattctgcaagcctccggagcgcacgtcggcagt | |
| | | cggctccctcgttgaccgaatcaccgacctctctccccagctgtatttccaaaatgtcgctttcta | |
| | | acaagctgacgctggacaagctggacgttaaagggaagcgggtcgttatgagagtcgacttc | |
| | | aatgttcctatgaagaacaaccagataacaaacaaccagaggattaaggctgctgtcccaagc | |
| | | atcaaattctgcttggacaatggagccaagtcggtagtccttatgagccacctaggccggcct | |
| | | gatggtgtgcccatgcctgacaagtactccttagagccagttgctgtagaactcaaatctctgct | |
| | | gggcaaggatgttctgttcttgaaggactgtgtaggcccagaagtggagaaagcctgtgcca | |
| | | acccagctgctgggtctgtcatcctgctggagaacctccgctttcatgtggaggaagaaggga | |
| | | agggaaaagatgcttctgggaacaaggttaaagccgagccagccaaaatagaagctttccga | |
| | | gcttcactttccaagctaggggatgtctatgtcaatgatgcttttggcactgctcacagagccca | |
| | | cagctccatggtaggagtcaatctgccacagaaggctggtgggtttttgatgaagaaggagct | |
| | | gaactactttgcaaaggccttggagagcccagagcgacccttcctggccatcctgggcggag | |
| | | ctaaagttgcagacaagatccagctcatcaataatatgctggacaaagtcaatgagatgattatt | |
| | | ggtggtggaatggcttttaccttccttaaggtgctcaacaacatggagattggcacttctctgttt | |
| | | gatgaagagggagccaagattgtcaaagacctaatgtccaaagctgagaagaatggtgtgaa | |
| | | gattaccttgcctgttgactttgtcactgctgacaagtttgatgagaatgccaagactggccaag | |
| | | ccactgtggcttctggcatacctgctggctggatgggcttggactgtggtcctgaaagcagca | |
| | | agaagtatgctgaggctgtcactcgggctaagcagattgtgtggaatggtcctgtgggggtatt | |
| | | tgaatgggaagcttttgcccggggaaccaaagctctcatggatgaggtggtgaaagccacttc | |
| | | taggggctgcatcaccatcataggtggtggagacactgccacttgctgtgccaaatggaaca | |
| | | cggaggataaagtcagccatgtgagcactgggggtggtgccagtttggagctcctggaaggt | |
| | | aaagtccttcctggggtggatgctctcagcaatatttagtactttcctgccttttagttcctgtgca | |
| | | cagcccctaagtcaacttagcattttctgcatctccacttggcattagctaaaaccttccatgtca | |
| | | agattcagctagtggccaagagatgcagtgccaggaacccttaaacagttgcacagcatctca | |
| | | gctcatcttcactgcaccctggatttgcatacattcttcaagatcccatttgaattttttagtgacta | |
| | | aaccattgtgcattctagagtgcatatatttatattttgcctgttaaaaagaaagtgagcagtgtta | |
| | | gcttagttctcttttgatgtaggttattatgattagctttgtcactgtttcactactcagcatggaaac | |
| | | aagatgaaattccatttgtaggtagtgagacaaaattgatgatccattaagtaaacaataaaagt | |
| | | gtccattgaaaccgtgatttttttttttttcctgtcatactttgttaggaagggtgagaatagaatctt | |
| | | gaggaacggatcagatgtctatattgctgaatgcaagaagtggggcagcagcagtggagag | |
| | | atgggacaattagataaatgtccattctttatcaagggcctactttatggcagacattgtgctagt | |
| | | gcttttattctaacttttatttttatcagttacacatgatcataatttaaaaagtcaaggcttataacaa | |
| | | aaaagccccagcccattcctcccattcaagattcccactccccagaggtgaccactttcaactc | |
| | | ttgagtttttcaggtatatacctccatgtttctaagtaatatgcttatattgttcacttcttttttttttatttt | |
| | | ttaaagaaatctatttcataccatggaggaaggctctgttccacatatatttccacttcttcattctct | |
| | | cggtatagttttgtcacaattatagattagatcaaaagtctacataactaatacagctgagctatgt | |
| | | agtatgctatgattaaatttacttatgtaaaaaaaaaaaaaaaaaa | |
| RPL13A | NM_012423.3 | cacttctgccgcccctgtttcaagggataagaaaccctgcgacaaaacctcctccttttccaag | 59 |
| | | cggctgccgaagatggcggaggtgcaggtcctggtgcttgatggtcgaggccatctcctgg | |
| | | gccgcctggcggccatcgtggctaaacaggtactgctgggccggaaggtggtggtcgtacg | |
| | | ctgtgaaggcatcaacatttctggcaatttctacagaaacaagttgaagtacctggctttcctcc | |
| | | gcaagcggatgaacaccaacccttcccgaggcccctaccacttccgggcccccagccgcat | |
| | | cttctggcggaccgtgcgaggtatgctgccccacaaaaccaagcgaggccaggccgctctg | |
| | | gaccgtctcaaggtgtttgacggcatcccaccgccctacgacaagaaaaagcggatggtggt | |
| | | tcctgctgccctcaaggtcgtgcgtctgaagcctacaagaaagtttgcctatctggggcgcctg | |
| | | gctcacgaggttggctggaagtaccaggcagtgacagccaccctggaggagaagaggaaa | |
| | | gagaaagccaagatccactaccggaagaagaaacagctcatgaggctacggaaacaggcc | |
| | | gagaagaacgtggagaagaaaattgacaaatacacagaggtcctcaagacccacggactcc | |
| | | tggtctgagcccaataaagactgttaattcctcatgcgttgcctgcccttcctccattgttgccct | |
| | | ggaatgtacgggacccaggggcagcagcagtccaggtgccacaggcagccctgggacat | |
| | | aggaagctgggagcaaggaaagggtcttagtcactgcctcccgaagttgcttgaaagcactc | |
| | | ggagaattgtgcaggtgtcatttatctatgaccaataggaagagcaaccagttactatgagtga | |
| | | aagggagccagaagactgattggagggccctatcttgtgagtggggcatctgttggactttcc | |
| | | acctggtcatatactctgcagctgttagaatgtgcaagcacttggggacagcatgagcttgctg | |
| | | ttgtacacagggtatttctagaagcagaaatagactgggaagatgcacaaccaaggggttaca | |
| | | ggcatcgcccatgctcctcacctgtattttgtaatcagaaataaattgcttttaaagaaaaaaaaa | |
| | | aaaaaaaaa | |
| B2M | NM_004048.2 | aatataagtggaggcgtcgcgctggcgggcattcctgaagctgacagcattcgggccgagat | 60 |
| | | gtctcgctccgtggccttagctgtgctcgcgctactctctctttctggcctggaggctatccagc | |
| | | gtactccaaagattcaggtttactcacgtcatccagcagagaatggaaagtcaaatttcctgaat | |
| | | tgctatgtgtctgggtttcatccatccgacattgaagttgacttactgaagaatggagagagaatt | |
| | | gaaaaagtggagcattcagacttgtctttcagcaaggactggtctttctatctcttgtactacact | |
| | | gaattcacccccactgaaaaagatgagtatgcctgccgtgtgaaccatgtgactttgtcacagc | |
| | | ccaagatagttaagtgggatcgagacatgtaagcagcatcatggaggtttgaagatgccgcat | |
| | | ttggattggatgaattccaaattctgcttgcttgctttttaatattgatatgcttatacacttacacttta | |
| | | tgcacaaaatgtagggttataataatgttaacatggacatgatcttctttataattctactttgagtg | |
| | | ctgtctccatgtttgatgtatctgagcaggttgctccacaggtagctctaggagggctggcaact | |
| | | tagaggtggggagcagagaattctcttatccaacatcaacatcttggtcagatttgaactcttca | |
| | | atctcttgcactcaaagcttgttaagatagttaagcgtgcataagttaacttccaatttacatactct | |
| | | gcttagaatttgggggaaaatttagaaatataattgacaggattattggaaatttgttataatgaat | |
| | | gaaacattttgtcatataagattcatatttacttcttatacatttgataaagtaaggcatggttgtggt | |
| | | taatctggtttatttttgttccacaagttaaataaatcataaaacttgatgtgttatctctta | |
| YWHAZ | NM_003406.3 | ctttctccttccccttcttccgggctcccgtcccggctcatcacccggcctgtggcccactccca | 61 |
| | | ccgccagctggaaccctggggactacgacgtccctcaaaccttgcttctaggagataaaaag | |
| | | aacatccagtcatggataaaaatgagctggttcagaaggccaaactggccgagcaggctga | |
| | | gcgatatgatgacatggcagcctgcatgaagtctgtaactgagcaaggagctgaattatccaa | |
| | | tgaggagaggaatcttctctcagttgcttataaaaatgttgtaggagcccgtaggtcatcttgga | |
| | | gggtcgtctcaagtattgaacaaaagacggaaggtgctgagaaaaaacagcagatggctcg | |
| | | agaatacagagagaaaattgagacggagctaagagatatctgcaatgatgtactgtctcttttg | |
| | | gaaaagttcttgatccccaatgcttcacaagcagagagcaaagtcttctatttgaaaatgaaag | |
| | | gagattactaccgttacttggctgaggttgccgctggtgatgacaagaaagggattgtcgatca | |
| | | gtcacaacaagcataccaagaagcttttgaaatcagcaaaaaggaaatgcaaccaacacatc | |
| | | ctatcagactgggtctggcccttaacttctctgtgttctattatgagattctgaactccccagaga | |
| | | aagcctgctctcttgcaaagacagcttttgatgaagccattgctgaacttgatacattaagtgaa | |
| | | gagtcatacaaagacagcacgctaataatgcaattactgagagacaacttgacattgtggacat | |
| | | cggatacccaaggagacgaagctgaagcaggagaaggaggggaaaattaaccggccttcc | |
| | | aacttttgtctgcctcattctaaaatttacacagtagaccatttgtcatccatgctgtcccacaaata | |
| | | gttttttgtttacgatttatgacaggtttatgttacttctatttgaatttctatatttcccatgtggtttttat | |
| | | gtttaatattaggggagtagagccagttaacatttagggagttatctgttttcatcttgaggtggc | |
| | | caatatggggatgtggaatttttatacaagttataagtgtttggcatagtacttttggtacattgtgg | |
| | | cttcaaaagggccagtgtaaaactgcttccatgtctaagcaaagaaaactgcctacatactggt | |
| | | ttgtcctggcggggaataaaagggatcattggttccagtcacaggtgtagtaattgtgggtactt | |
| | | taaggtttggagcacttacaaggctgtggtagaatcataccccatggataccacatattaaacc | |
| | | atgtatatctgtggaatactcaatgtgtacacctttgactacagctgcagaagtgttcctttagac | |
| | | aaagttgtgacccattttactctggataagggcagaaacggttcacattccattatttgtaaagtt | |
| | | acctgctgttagctttcattatttttgctacactcattttatttgtatttaaatgttttaggcaacctaag | |
| | | aacaaatgtaaaagtaaagatgcaggaaaaatgaattgcttggtattcattacttcatgtatatca | |
| | | agcacagcagtaaaacaaaaacccatgtatttaacttttttttaggatttttgcttttgtgattttttttt | |
| | | ttttgatacttgcctaacatgcatgtgctgtaaaaatagttaacagggaaataacttgagatgatg | |
| | | gctagctttgtttaatgtcttatgaaattttcatgaacaatccaagcataattgttaagaacacgtgt | |
| | | attaaattcatgtaagtggaataaaagttttatgaatggacttttcaactactttctctacagcttttc | |
| | | atgtaaattagtcttggttctgaaacttctctaaaggaaattgtacattttttgaaatttattccttattc | |
| | | cctcttggcagctaatgggctcttaccaagtttaaacacaaaatttatcataacaaaaatactact | |
| | | aatataactactgtttccatgtcccatgatcccctctcttcctccccaccctgaaaaaaatgagttc | |
| | | ctattttttctgggagagggggggattgattagaaaaaaatgtagtgtgttccatttaaaattttgg | |
| | | catatggcattttctaacttaggaagccacaatgttcttggcccatcatgacattgggtagcatta | |
| | | actgtaagttttgtgcttccaaatcactttttggtttttaagaatttcttgatactcttatagcctgcctt | |
| | | caattttgatcctttattctttctatttgtcaggtgcacaagattaccttcctgttttagccttctgtctt | |
| | | gtcaccaaccattcttacttggtggccatgtacttggaaaaaggccgcatgatctttctggctcc | |
| | | actcagtgtctaaggcaccctgcttcctttgcttgcatcccacagactatttccctcatcctatttac | |
| | | tgcagcaaatctctccttagttgatgagactgtgtttatctccctttaaaaccctacctatcctgaat | |
| | | ggtctgtcattgtctgcctttaaaatccttcctctttcttcctcctctattctctaaataatgatgggg | |
| | | ctaagttatacccaaagctcactttacaaaatatttcctcagtactttgcagaaaacaccaaacaa | |
| | | aaatgccattttaaaaaaggtgtattttttcttttagaatgtaagctcctcaagagcagggacaat | |
| | | gttttctgtatgttctattgtgcctagtacactgtaaatgctcaataaatattgatgatgggaggca | |
| | | gtgagtcttgatgataagggtgagaaactgaaatcccaaacactgttttgttgcttgttttattatg | |
| | | acctcagattaaattgggaaatattggcccttttgaataattgtcccaaatattacattcaaataaa | |
| | | agtgcaatggagaaaaaaaaaaa | |
| SDHA | NM_004168.3 | actgcagccccgctcgactccggcgtggtgcgcaggcgcggtatcccccctcccccgccag | 62 |
| | | ctcgaccccggtgtggtgcgcaggcgcagtctgcgcagggactggcgggactgcgcggc | |
| | | ggcaacagcagacatgtcgggggtccggggcctgtcgcggctgctgagcgctcggcgcct | |
| | | ggcgctggccaaggcgtggccaacagtgttgcaaacaggaacccgaggttttcacttcactg | |
| | | ttgatgggaacaagagggcatctgctaaagtttcagattccatttctgctcagtatccagtagtg | |
| | | gatcatgaatttgatgcagtggtggtaggcgctggaggggcaggcttgcgagctgcatttggc | |
| | | ctttctgaggcagggtttaatacagcatgtgttaccaagctgtttcctaccaggtcacacactgtt | |
| | | gcagcacagggaggaatcaatgctgctctggggaacatggaggaggacaactggaggtgg | |
| | | catttctacgacaccgtgaagggctccgactggctgggggaccaggatgccatccactacat | |
| | | gacggagcaggcccccgccgccgtggtcgagctagaaaattatggcatgccgtttagcaga | |
| | | actgaagatgggaagatttatcagcgtgcatttggtggacagagcctcaagtttggaaagggc | |
| | | gggcaggcccatcggtgctgctgtgtggctgatcggactggccactcgctattgcacacctta | |
| | | tatggaaggtctctgcgatatgataccagctattttgtggagtattttgccttggatctcctgatgg | |
| | | agaatggggagtgccgtggtgtcatcgcactgtgcatagaggacgggtccatccatcgcata | |
| | | agagcaaagaacactgttgttgccacaggaggctacgggcgcacctacttcagctgcacgtc | |
| | | tgcccacaccagcactggcgacggcacggccatgatcaccagggcaggccttccttgccag | |
| | | gacctagagtttgttcagttccaccctacaggcatatatggtgctggttgtctcattacggaagg | |
| | | atgtcgtggagagggaggcattctcattaacagtcaaggcgaaaggtttatggagcgatacg | |
| | | cccctgtcgcgaaggacctggcgtctagagatgtggtgtctcggtccatgactctggagatcc | |
| | | gagaaggaagaggctgtggccctgagaaagatcacgtctacctgcagctgcaccacctacct | |
| | | ccagagcagctggccacgcgcctgcctggcatttcagagacagccatgatcttcgctggcgt | |
| | | ggacgtcacgaaggagccgatccctgtcctccccaccgtgcattataacatgggcggcattc | |
| | | ccaccaactacaaggggcaggtcctgaggcacgtgaatggccaggatcagattgtgcccgg | |
| | | cctgtacgcctgtggggaggccgcctgtgcctcggtacatggtgccaaccgcctcggggca | |
| | | aactcgctcttggacctggttgtctttggtcgggcatgtgccctgagcatcgaagagtcatgca | |
| | | ggcctggagataaagtccctccaattaaaccaaacgctggggaagaatctgtcatgaatcttg | |
| | | acaaattgagatttgctgatggaagcataagaacatcggaactgcgactcagcatgcagaagt | |
| | | caatgcaaaatcatgctgccgtgttccgtgtgggaagcgtgttgcaagaaggttgtgggaaaa | |
| | | tcagcaagctctatggagacctaaagcacctgaagacgttcgaccggggaatggtctggaac | |
| | | acggacctggtggagaccctggagctgcagaacctgatgctgtgtgcgctgcagaccatcta | |
| | | cggagcagaggcacggaaggagtcacggggcgcgcatgccagggaagactacaaggtg | |
| | | cggattgatgagtacgattactccaagcccatccaggggcaacagaagaagccctttgagga | |
| | | gcactggaggaagcacaccctgtcctatgtggacgttggcactgggaaggtcactctggaat | |
| | | atagacccgtgatcgacaaaactttgaacgaggctgactgtgccaccgtcccgccagccattc | |
| | | gctcctactgatgagacaagatgtggtgatgacagaatcagcttttgtaattatgtataatagctc | |
| | | atgcatgtgtccatgtcataactgtcttcatacgcttctgcactctggggaagaaggagtacatt | |
| | | gaagggagattggcacctagtggctgggagcttgccaggaacccagtggccagggagcgt | |
| | | ggcacttacctttgtcccttgcttcattcttgtgagatgataaaactgggcacagctcttaaataaa | |
| | | atataaatgaacaaactttcttttatttccaaatccatttgaaatattttactgttgtgactttagtcata | |
| | | tttgttgacctaaaaatcaaatgtaatctttgtattgtgttacatcaaaatccagatattttgtatagtt | |
| | | tcttttttctttttcttttcttttttttttttgagacaggatcggtgcagtagtacaatcacagctcactgc | |
| | | agcctcaaactcctgggcagctcaggtgatcttcctgactcagccttctgagtagttggggcta | |
| | | caggtgtgcaccaccatgcccagctcatttattttgtaattgtagggacagggtctcactgtgttg | |
| | | cctaggctggtctcaagtgatcctccctccttggcctcccaaggtgctggaattataggtgtga | |
| | | acaaaccaaaaaaaaaaaaaa | |
| HPRT1 | NM_000194.2 | ggcggggcctgcttctcctcagcttcaggcggctgcgacgagccctcaggcgaacctctcg | 63 |
| | | gctttcccgcgcggcgccgcctcttgctgcgcctccgcctcctcctctgctccgccaccggctt | |
| | | cctcctcctgagcagtcagcccgcgcgccggccggctccgttatggcgacccgcagccctg | |
| | | gcgtcgtgattagtgatgatgaaccaggttatgaccttgatttattttgcatacctaatcattatgct | |
| | | gaggatttggaaagggtgtttattcctcatggactaattatggacaggactgaacgtcttgctcg | |
| | | agatgtgatgaaggagatgggaggccatcacattgtagccctctgtgtgctcaaggggggct | |
| | | ataaattctttgctgacctgctggattacatcaaagcactgaatagaaatagtgatagatccattc | |
| | | ctatgactgtagattttatcagactgaagagctattgtaatgaccagtcaacaggggacataaa | |
| | | agtaattggtggagatgatctctcaactttaactggaaagaatgtcttgattgtggaagatataat | |
| | | tgacactggcaaaacaatgcagactttgctttccttggtcaggcagtataatccaaagatggtc | |
| | | aaggtcgcaagcttgctggtgaaaaggaccccacgaagtgttggatataagccagactttgtt | |
| | | ggatttgaaattccagacaagtttgttgtaggatatgcccttgactataatgaatacttcagggatt | |
| | | tgaatcatgtttgtgtcattagtgaaactggaaaagcaaaatacaaagcctaagatgagagttc | |
| | | aagttgagtttggaaacatctggagtcctattgacatcgccagtaaaattatcaatgttctagttct | |
| | | gtggccatctgcttagtagagctttttgcatgtatcttctaagaattttatctgttttgtactttagaaa | |
| | | tgtcagttgctgcattcctaaactgtttatttgcactatgagcctatagactatcagttccctttggg | |
| | | cggattgttgtttaacttgtaaatgaaaaaattctcttaaaccacagcactattgagtgaaacattg | |
| | | aactcatatctgtaagaaataaagagaagatatattagttttttaattggtattttaatttttatatatg | |
| | | caggaaagaatagaagtgattgaatattgttaattataccaccgtgtgttagaaaagtaagaag | |
| | | cagtcaattttcacatcaaagacagcatctaagaagttttgttctgtcctggaattattttagtagtg | |
| | | tttcagtaatgttgactgtattttccaacttgttcaaattattaccagtgaatctttgtcagcagttcc | |
| | | cttttaaatgcaaatcaataaattcccaaaaatttaaaaaaaaaaaaaaaaaaaaaa | |
| TOX4 | NM_001303523.1 | agcagagagaacacacgtccttgcggaagtgacggcagttccgagtccagtgggggcggt | 64 |
| | | gggagcgatgagggtctgagacggtgggagcggttgtgtgaagatggagacattccataca | |
| | | ccaagcttgggtgatgaggaatttgaaatcccacctatctccttggattctgatccctcattggct | |
| | | gtctcagatgtggttggccactttgatgacctggcagacccttcctcttcacaggatggcagtttt | |
| | | tcagcccagtatggggtccagacattggacatgcctgtgggcatgacccatggcttgatgga | |
| | | gcagggcggggggctcctgagtgggggcttgaccatggacttggaccactctataggaact | |
| | | cagtatagtgccaacccacctgttacaattgatgtaccaatgacagacatgacatctggcttgat | |
| | | ggggcatagccagttgaccaccattgatcagtcagaactgagttcccagctgggtttgagcct | |
| | | agggggtggcaccatcctgccacctgcccagtcacctgaagatcgtctttcaaccaccccttc | |
| | | acctactagttcacttcacgaggatggtgttgaggatttccggaggcaacttcccagccagaa | |
| | | gacagtcgtggtggaagcagggaaaaagcagaaggccccaaagaagagaaaaaagaaag | |
| | | atcctaatgaacctcagaaaccagtttcagcatatgctttattctttcgtgatacacaggctgccat | |
| | | caagggacagaatcctaatgccacttttggtgaggtttcaaaaattgtggcctccatgtgggat | |
| | | agtcttggagaggagcaaaaacaggtatataagaggaaaactgaggctgccaagaaagagt | |
| | | atctgaaggcactggctgcttacaaagacaaccaggagtgtcaggccactgtggaaacagtg | |
| | | gaattggatccagcaccaccatcacaaactccttctccacctcctatggctactgttgacccag | |
| | | catctccagcaccagcttcaatagagccccctgccctgtccccatccattgttgttaactccacc | |
| | | ctttcatcctatgtggcaaaccaggcatcttctggagctgggggtcagcccaatatcaccaagt | |
| | | tgattattaccaaacaaatgttgccctcttctattactatgtctcaaggagggatggttactgttatc | |
| | | ccagccacagtggtgacctcccgggggctccaactaggccaaaccagtacagctactatcca | |
| | | gcccagtcaacaagcccagattgtcactcggtcagtgttgcaggcagcagcagctgctgctg | |
| | | ctgctgcttctatgcaactgcctccaccccgactacagccccctccattacaacagatgccaca | |
| | | gcccccgactcagcagcaagttaccattctgcagcagcctcctccactccaggccatgcaac | |
| | | agcctccacctcagaaagttcgaatcaatttacagcaacagcctcctcctctgcagatcaagag | |
| | | tgtgcctctacccactttgaaaatgcagactaccttagtcccaccaactgtggaaagtagtcctg | |
| | | agcggcctatgaacaacagccctgaggcccatacagtggaggcaccttctcctgagactatct | |
| | | gtgagatgatcacagatgtagttcctgaggttgagtctccttctcagatggatgttgaattggtga | |
| | | gtgggtctcctgtggcactctcaccccagcctcgatgtgtgaggtctggttgtgagaaccctcc | |
| | | cattgtgagtaaggactgggacaatgaatactgcagcaatgagtgtgtggtgaagcactgca | |
| | | gggatgtattcttggcctgggtagcctctagaaattcaaacacagtggtgtttgtgaaatagtcc | |
| | | ttcctgttctccaagccagtgaagagttatctgctgggaaagtgtccaagagcctgtttttgaaa | |
| | | cacaagctgggcttctggtagtgcctcatcacaacccatgatggctgttcatgtttcacccctttt | |
| | | cttccttcagcagaggccaggctatggagcagggccactgaatttgctgtaatctggagatgc | |
| | | tttttactttcaaccataagcggtaatagcagaggaaagggtgaagggagtctgggcaagcaa | |
| | | agcatagagatggtggggtggtggtggggttgaagaaacttgttggtataattgtcataggact | |
| | | tgcctaaaatattattaaaattacgggagtgtactcagctttgagcctaggagaaaatgccactg | |
| | | tgtgcatccattttaaagggttccctcataaaaaaatgttattccccattatcacatcagtacactg | |
| | | ctttgaaaacaaaacttttcaacatgggcatactgggctacatggaaaatgacatcacccagga | |
| | | gtgatttctctttatatatattatttctgcagttaccatccttatctgagttatcacagttcatgaatcta | |
| | | agaggcggaactctacatcattagtaagaggttccaccaaagtctaaagttgtattcacttgtgtt | |
| | | tgatgaactatctttaaaagaccataggtctatcattatttcttagacataatctaaagaaaaacag | |
| | | actagagaagccacctggttgtaacagaataagcagaagtttacagcatgatagtccaagtgg | |
| | | tgataactttaaataaaactcaaatttttactgtttgtagacaggaatgctgtcctagagaacctcc | |
| | | tcctcaaccagctacgtacatagttttatcctatgcattcctgttttctgtgtgttttttgttttttttttttt | |
| | | tttttttttttgagacagagtctcgctctgtcacccaggctggagtgcagtggtgcgacctcagct | |
| | | cactgaaacctctgcctcccgggttcaagcgattctcctgcatcagcctcccgagtagctagg | |
| | | attacaggcgcccgccactacgcccagctaatttgtggtatttttagtagagacagggtttcacc | |
| | | atgttggccaggctggtctcgaactcctgacctcatgatccgcccgccttgacctcccaaagtg | |
| | | ctgggattacaggcatgagccaccgcacccagcctgcattcctgtttttttaatggttttggagg | |
| | | gtagcagtagagatggggtctcactatgttgcccagtctagtcttgaactcctgggctacagtta | |
| | | ccctcctacctcggcttcccaaagtgctcggattacaggtgtgagccactgtgcctagcctata | |
| | | atgatcattttaatgtttcccatgcactcatttagtttgaaccttcacagcaacccaatgaggtaat | |
| | | actcccatttcacatataatactgagagatgagttgcacaagattatacactgttaagtagcaga | |
| | | gccagaatggacttcagaatcccaactacaatacaaatgtttatttaaataaagaagaaagctat | |
| | | tgtacaaatatcactcttcaggtttagcttacagagccatggctatggattcttagctctgtaagg | |
| | | aagtgcttctataaattcttaggtttagagatgataccatctgggtacctttgcttgaaccgtgcaa | |
| | | ccacatctgggtctagtaggtggatcccatccagttggtttccaagggtgatcctgaaacagtg | |
| | | taaaaggaggggcaaaccagaaatcctggaattagagggtttaatattgttaaaaaatgcatac | |
| | | caaatgaagactgcctatcatcatatcaaatatgccaattctaaaaagagcttaacattagaata | |
| | | gtatatggtagaattactagttcagaattggcatagattctggtgttaaaatagactggatctgtat | |
| | | tatctgagggttagtaactaatgcttagccaggcctgcttcacagagttgctaccagggagtatt | |
| | | ctttggataagcaaaatgctagcagcatgtgttttaagctctgttaaggggtgaaagatgtaatta | |
| | | ttgacagattaaatagataacttcgtaaccaccagggggcagattcaatacatcacagaatggc | |
| | | tgaggaagatccttgggttgtgaagagagtagaaaccctagggagcagtgcttttgggtccta | |
| | | gaacctgttgagtttctaatgaatatttgtagaatctcataaaacagtttaaatacaagcttaagtg | |
| | | gcttatgaatcctgtgaagctcatttatggactagtgtaaaacaatgtgaagctctactaagttct | |
| | | gtccttaatcataaataatagccccttgaggactagcctgttctctggtcaccttaccagttgggt | |
| | | tgcacattgtgtggtcgtccaaataactcaatcttgcgagtgccaggagatagtctttcaatcat | |
| | | gccatagatttcatctggtttatgactggtggaacgaacctaggaaataaaaactagctgcttttt | |
| | | aagttacacaagaaaaaa | |
| TPT1 | NM_001286272.1 | cttcgtgccacgtcaccgcctgcgtcgcttccggaggcgcagcgggcgatgacgtagaggg | 65 |
| | | acgtgccctctatatgaggttggggagcggctgagtcggccttttccgcccgctcccccctcc | |
| | | ccccgagcgccgctccggctgcaccgcgctcgctccgagtttcaggctcgtgctaagctagc | |
| | | gccgtcgtcgtctcccttcagtcgccatcatgattatctaccgggacctcatcagccacgatga | |
| | | gatgttctccgacatctacaagatccgggagatcgcggacgggttgtgcctggaggtggagg | |
| | | ggaagatggtcagtaggacagaaggtaacattgatgactcgctcattggtggaaatgcctccg | |
| | | ctgaaggccccgagggcgaaggtaccgaaagcacagtaatcactggtgtcgatattgtcatg | |
| | | aaccatcacctgcaggaaacaagtttcacaaaagaagcctacaagaagtacatcaaagattac | |
| | | atgaaatcaatcaaagggaaacttgaagaacagagaccagaaagagtaaaaccttttatgaca | |
| | | ggggctgcagaacaaatcaagcacatccttgctaatttcaaaaactaccagttctttattggtga | |
| | | aaacatgaatccagatggcatggttgctctattggactaccgtgaggatggtgtgaccccatat | |
| | | atgattttctttaaggatggtttagaaatggaaaaatgtgatgcaaaagaaagaaatccctgcgc | |
| | | tttctgtctgtctttgtggcggcccagattgaattggggaatacatctttagcctggaaatgtagg | |
| | | ctgcatgttaatggtaatgtaacttttgcagtgtaatgtttgaaaaatattaatgtagtttttgctttta | |
| | | cagtaacaaatgtggcaattattttggatctatcacctgtcatcataactggcttctgcttgtcatc | |
| | | cacacaacaccaggacttaagacaaatgggactgatgtcatcttgagctcttcatttattttgact | |
| | | gtgatttatttggagtggaggcattgtttttaagaaaaacatgtcatgtaggttgtctaaaaataaa | |
| | | atgcatttaaactcatttgagagaatgccttttagtttaatgcatatttaaactaaattgatcctgtag | |
| | | tgttcctggagaagctagagcctgattgtaggctactactcatcaattaacttctacagtggaga | |
| | | ctacttctgggactggaatataaaaaagaatcaaaggttctgattttgagttgcaataaagggaa | |
| | | agaccatgctcatagcagtgccaacatctgaagtgtggagccttacccatttcatcacctacaa | |
| | | cggaagtagttaactggaagagattaccaagagaataaaaagagactcattcagtggaagca | |
| | | actttgtctcagcttatttcacataaagagagcgaagtcttttgggatgaatgttaattaaactccc | |
| | | tggtaactagaacagggactggcaaactagcctatctgaccacctgttttgtacactttaaggtg | |
| | | gttggttgcctttttaaatggttgaggggaaaagaataccttgtgggatatggaatttaagttcga | |
| | | gtccagttttattggaacgtggctatgcttattcatttatggattgactgtggctgttgtcagtgcat | |
| | | gagcagagttgtgtctaacagactagagcctgcaagtttgccagcccctgatttaaaagatga | |
| | | aggtacacagaatgtgggctggctggtgggcaaaggggtaaaaatgttctctatattgtatctg | |
| | | aaaagatggggtgtctgaataagaaaatgcatctatttgacagacctggagcagttgctatctg | |
| | | ctgctatggtttccaccacagatgcaagaagaacatgtccttgcgctttccgtctgtctaattgtg | |
| | | gcagctgagattgaatagaggaatacaggaggaaaaaaagcgggaagagtttttgaggcag | |
| | | gtcggtcacccaggcttgtagtgcagtggcacaagcaactcactgcattctctgcatcctgtgc | |
| | | tcaagccattttcccacctcagtctcactagttgctgggactgcaggcatgcacccctatgccc | |
| | | agctaatttttgtagagaccgagtatcgcttagttgcccagggtggtctcaactcctgggctcaa | |
| | | ggagatctgcccacctcagcctcccaaagtgcaggcctagcctgggaggggaattttcaaaa | |
| | | cgtgagttttgggaaatagtctatcagccttacctggttgattacacttgtaaaagaaagattaaa | |
| | | agcaggccagtgactctggtctgcttgaacatgtgaatgtagtggtttgagcaatctggagtttg | |
| | | ccctagtgtcaaattccagactgtccatagtgtccaaaacctgaggcagatactaatgttaaccc | |
| | | ccagcaccccgtgattggaaacaaacctaaatacgtattgggaacttaatagcaattttaagcat | |
| | | tctgatagattttttgtagggatggggtcatgccatgtggcccaggctggtctgaaaactctggc | |
| | | ctcaagtgatctcaagctttggccttctaaagtgttgggattacaggtgtgaggcattgcacctg | |
| | | gcttagcgttctgatttgacattgtaatgaaaagtgtgagtctcatctacagggccttttgtcctct | |
| | | gaaatgatagcaggaagggaattttcaggcagtggtcaaagctggggaaaccaggatagtg | |
| | | aagaaggccttgaggtgagagatggaagctaattggtgaactagccttggaagcctgaaaca | |
| | | gacaagtagcaattcagagactttgtgggctccactgctccaacttgttttgaagattttcagttct | |
| | | gcagaagaggtatttccccagttgtcctttcagtgctcttagctgttttcccaacatccagatcca | |
| | | atcaaggctgggacatagcattttatcatgtctatttaagtcagaagtgatgaaccccagctgttt | |
| | | acctcatggtaaacctttgaagattccaggtagaatcttctcagactttgaagactgtctcatttta | |
| | | tatctttttctcgttattcctagggtcaagacgttttgggcaagaataaggatgtgaacatcagaa | |
| | | agctcataacattttgtttttgatgctaagtttaacaaaggcatgctttagtagcctgtgggcccta | |
| | | gggtttgttaaagtgtggagaacaactgagtggagcaagaggacttttctaggaaggtccttgt | |
| | | aatgtgacatttgaaaacaaatgaaggtgtggaagtaggccatgtggatatcaggacaaacca | |
| | | ttccaggccaagacaacagcagttagtctggagtgtgatgtgttctgggaaaaaagtggccac | |
| | | tttgctaacccaagaagacaggaagggttgtaaagcagtgggagtgtgcaaggaaggaaga | |
| | | ccagacctcaaggaaaccacaggcgctctgagcagaagagttacatgatatgactcaaattttt | |
| | | aaaggatcactttggctgccaggtggcagggtaaaagcatagaataattgtgtataatgtgtttt | |
| | | taaggcaaagatagtggcttagtctagggtagtagactgaggtggtaggaaatgaagataga | |
| | | gacaacaggatatgctggtgggtgaggatggatttaatgttgatacaagtattttggtctgagcg | |
| | | tttggaagaaagttggcactgaggtgggaagtcgagtttagttttgttagttttggatgtgttaagt | |
| | | ttgagatgctgattcttcagagaagtctaagctggagaactatatagagagtggaaagataaca | |
| | | atagacattgaaagccatgatacaggataaggtcatttggagagaggatagactgcattccaa | |
| | | catgagattggttgacaaagagaaaccaacaaaggtaattaagaggtgctcccactgcacttg | |
| | | tactcagaaggctgaggtaggattgttagaggccagcctgggcaccacagggagaccccat | |
| | | ctctaaaatttagccaggaaccatggctcatgcctgtagccccaggaatttgggaggctgagt | |
| | | ggggaggatcgcttgaggtcaggagtttgagaccagcctgggcaacatagggagacctaaa | |
| | | aaaattaattgggcatctgtagtcccagctactcaggcggctgagctgagaggatggcttgag | |
| | | tccgagagattgagggtgcagtgagctgtgatcataccactgcactccagcctgggcggcag | |
| | | tgagacactatctgaaaaaagtttaaaaattttaaaaaagaaggaactgcccctgaggtaagaa | |
| | | ccaagggagggcctcccagaggtcaggtggaaaaagttttaggaaggaggaagtagtcaa | |
| | | cagggttacctgttgcaaagtacttaagtaatatgaggcctgatagtggtaaacttgactaccgt | |
| | | tggatttcactagtgggaaaggaagtctaattaaaatgcactcaagagactaacagtcgcagg | |
| | | catgaaatacaatacaggtacatggttttttattatgtgtgcatctgcttcagtaataggtgtgaatt | |
| | | actcatttggatcattaggagtttcaaaatctagttaaatgactagatttttgttgatgtaaattctgt | |
| | | cattctgaactgcagggattgtcagtaacttaactgcaaactaaactggtgataattatggtaaa | |
| | | attgcaagacgagcaataaatctcaaccaacttgagagaacactgataa | |

### Definitions

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

As used herein, the terms “polynucleotide” and “nucleic acid molecule” are used interchangeably to mean a polymeric form of nucleotides of at least 10 bases or base pairs in length, either ribonucleotides or deoxynucleotides or a modified form of either type of nucleotide, and is meant to include single and double stranded forms of DNA. As used herein, a nucleic acid molecule or nucleic acid sequence that serves as a probe in a microarray analysis preferably comprises a chain of nucleotides, more preferably DNA and/or RNA. In other embodiments, a nucleic acid molecule or nucleic acid sequence comprises other kinds of nucleic acid structures such a for instance a DNA/RNA helix, peptide nucleic acid (PNA), locked nucleic acid (LNA) and/or a ribozyme. Hence, as used herein the term “nucleic acid molecule” also encompasses a chain comprising non-natural nucleotides, modified nucleotides and/or non-nucleotide building blocks which exhibit the same function as natural nucleotides.

As used herein, the terms “hybridize,” “hybridizing”, “hybridizes,” and the like, used in the context of polynucleotides, are meant to refer to conventional hybridization conditions, such as hybridization in 50% formamide/6×SSC/0.1% SDS/100 μg/ml ssDNA, in which temperatures for hybridization are above 37 degrees centigrade and temperatures for washing in 0.1×SSC/0.1% SDS are above 55 degrees C., and preferably to stringent hybridization conditions.

As used herein, the term “normalization” or “normalizer” refers to the expression of a differential value in terms of a standard value to adjust for effects which arise from technical variation due to sample handling, sample preparation, and measurement methods rather than biological variation of biomarker concentration in a sample. For example, when measuring the expression of a differentially expressed protein, the absolute value for the expression of the protein can be expressed in terms of an absolute value for the expression of a standard protein that is substantially constant in expression.

The terms “diagnosis” and “diagnostics” also encompass the terms “prognosis” and “prognostics”, respectively, as well as the applications of such procedures over two or more time points to monitor the diagnosis and/or prognosis over time, and statistical modeling based thereupon. Furthermore, the term diagnosis includes: a. prediction (determining if a patient will likely develop aggressive disease (hyperproliferative/invasive)), b. prognosis (predicting whether a patient will likely have a better or worse outcome at a pre-selected time in the future), c. therapy selection, d. therapeutic drug monitoring, and e. relapse monitoring.

“Accuracy” refers to the degree of conformity of a measured or calculated quantity (a test reported value) to its actual (or true) value. Clinical accuracy relates to the proportion of true outcomes (true positives (TP) or true negatives (TN)) versus misclassified outcomes (false positives (FP) or false negatives (FN)), and may be stated as a sensitivity, specificity, positive predictive values (PPV) or negative predictive values (NPV), or as a likelihood, odds ratio, among other measures.

The term “biological sample” as used herein refers to any sample of biological origin potentially containing one or more biomarkers. Examples of biological samples include tissue, organs, or bodily fluids such as whole blood, plasma, serum, tissue, lavage or any other specimen used for detection of disease.

The term “subject” as used herein refers to a mammal, preferably a human. In some embodiments, the subject has at least one prostate cancer symptom. In some embodiments, the subject has a predisposition or familial history for developing a prostate cancer. The subject could also be previously diagnosed with a prostate cancer and is tested for cancer recurrence. In some embodiments, the subject has benign prostate hyperplasia.

“Treating” or “treatment” as used herein with regard to a condition may refer to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof.

Biomarker levels may change due to treatment of the disease. The changes in biomarker levels may be measured by the present disclosure. Changes in biomarker levels may be used to monitor the progression of disease or therapy.

“Altered”, “changed” or “significantly different” refer to a detectable change or difference from a reasonably comparable state, profile, measurement, or the like. Such changes may be all or none. They may be incremental and need not be linear. They may be by orders of magnitude. A change may be an increase or decrease by 5%, 10%, 20%, 30%, 40%, 50%, 60%, 70%, 80%, 90%, 95%, 99%, 100%, or more, or any value in between 0% and 100%. Alternatively, the change may be 1-fold, 1.5-fold, 2-fold, 3-fold, 4-fold, 5-fold or more, or any values in between 1-fold and 5-fold. The change may be statistically significant with a p value of 0.1, 0.05, 0.001, or 0.0001.

The term “stable disease” refers to a diagnosis for the presence of a prostate cancer, however the prostate cancer has been treated and remains in a stable condition, i.e. one that that is not progressive, as determined by imaging data and/or best clinical judgment.

The term “progressive disease” refers to a diagnosis for the presence of a highly active state of a prostate cancer, i.e. one has not been treated and is not stable or has been treated and has not responded to therapy, or has been treated and active disease remains, as determined by imaging data and/or best clinical judgment.

The term “neoplastic disease” refers to any abnormal growth of cells or tissues being either benign (non-cancerous) or malignant (cancerous). For example, the neoplastic disease can be a prostate cancer.

The term “neoplastic tissue” refers to a mass of cells that grow abnormally.

The term “non-neoplastic tissue” refers to a mass of cells that grow normally.

The term “immunotherapy” can refer to activating immunotherapy or suppressing immunotherapy. As will be appreciated by those in the art, activating immunotherapy refers to the use of a therapeutic agent that induces, enhances, or promotes an immune response, including, e.g., a T cell response while suppressing immunotherapy refers to the use of a therapeutic agent that interferes with, suppresses, or inhibits an immune response, including, e.g., a T cell response. Activating immunotherapy may comprise the use of checkpoint inhibitors.

Activating immunotherapy may comprise administering to a subject a therapeutic agent that activates a stimulatory checkpoint molecule. Stimulatory checkpoint molecules include, but are not limited to, CD27, CD28, CD40, CD122, CD137, OX40, GITR and ICOS. Therapeutic agents that activate a stimulatory checkpoint molecule include, but are not limited to, MEDI0562, TGN1412, CDX-1127, lipocalin.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An antibody that binds to a target refers to an antibody that is capable of binding the target with sufficient affinity such that the antibody is useful as a diagnostic and/or therapeutic agent in targeting the target. In one embodiment, the extent of binding of an anti-target antibody to an unrelated, non-target protein is less than about 10% of the binding of the antibody to target as measured, e.g., by a radioimmunoassay (RIA) or biacore assay. In certain embodiments, an antibody that binds to a target has a dissociation constant (Kd) of <1 μM, <100 nM, <10 nM, <1 nM, <0.1 nM, <0.01 nM, or <0.001 nM (e.g. 108 M or less, e.g. from 108 M to 1013 M, e.g., from 109 M to 1013 M). In certain embodiments, an anti-target antibody binds to an epitope of a target that is conserved among different species.

A “blocking antibody” or an “antagonist antibody” is one that partially or fully blocks, inhibits, interferes, or neutralizes a normal biological activity of the antigen it binds. For example, an antagonist antibody may block signaling through an immune cell receptor (e.g., a T cell receptor) so as to restore a functional response by T cells (e.g., proliferation, cytokine production, target cell killing) from a dysfunctional state to antigen stimulation.

An “agonist antibody” or “activating antibody” is one that mimics, promotes, stimulates, or enhances a normal biological activity of the antigen it binds. Agonist antibodies can also enhance or initiate signaling by the antigen to which it binds. In some embodiments, agonist antibodies cause or activate signaling without the presence of the natural ligand. For example, an agonist antibody may increase memory T cell proliferation, increase cytokine production by memory T cells, inhibit regulatory T cell function, and/or inhibit regulatory T cell suppression of effector T cell function, such as effector T cell proliferation and/or cytokine production.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

Administering chemotherapy to a subject can comprise administering a therapeutically effective dose of at least one chemotherapeutic agent. Chemotherapeutic agents include, but are not limited to, 13-cis-Retinoic Acid, 2-CdA, 2-Chlorodeoxyadenosine, 5-Azacitidine, 5-Fluorouracil, 5-FU, 6-Mercaptopurine, 6-MP, 6-TG, 6-Thioguanine, Abemaciclib, Abiraterone acetate, Abraxane, Accutane, Actinomycin-D, Adcetris, Ado-Trastuzumab Emtansine, Adriamycin, Adrucil, Afatinib, Afinitor, Agrylin, Ala-Cort, Aldesleukin, Alemtuzumab, Alecensa, Alectinib, Alimta, Alitretinoin, Alkaban-AQ, Alkeran, All-transretinoic Acid, Alpha Interferon, Altretamine, Alunbrig, Amethopterin, Amifostine, Aminoglutethimide, Anagrelide, Anandron, Anastrozole, Apalutamide, Arabinosylcytosine, Ara-C, Aranesp, Aredia, Arimidex, Aromasin, Arranon, Arsenic Trioxide, Arzerra, Asparaginase, Atezolizumab, Atra, Avastin, Avelumab, Axicabtagene Ciloleucel, Axitinib, Azacitidine, Bavencio, Bcg, Beleodaq, Belinostat, Bendamustine, Bendeka, Besponsa, Bevacizumab, Bexarotene, Bexxar, Bicalutamide, Bicnu, Blenoxane, Bleomycin, Blinatumomab, Blincyto, Bortezomib, Bosulif, Bosutinib, Brentuximab Vedotin, Brigatinib, Busulfan, Busulfex, C225, Cabazitaxel, Cabozantinib, Calcium Leucovorin, Campath, Camptosar, Camptothecin-11, Capecitabine, Caprelsa, Carac, Carboplatin, Carfilzomib, Carmustine, Carmustine Wafer, Casodex, CCI-779, Ccnu, Cddp, Ceenu, Ceritinib, Cerubidine, Cetuximab, Chlorambucil, Cisplatin, Citrovorum Factor, Cladribine, Clofarabine, Clolar, Cobimetinib, Cometriq, Cortisone, Cosmegen, Cotellic, Cpt-11, Crizotinib, Cyclophosphamide, Cyramza, Cytadren, Cytarabine, Cytarabine Liposomal, Cytosar-U, Cytoxan, Dabrafenib, Dacarbazine, Dacogen, Dactinomycin, Daratumumab, Darbepoetin Alfa, Darzalex, Dasatinib, Daunomycin, Daunorubicin, Daunorubicin Cytarabine (Liposomal), daunorubicin-hydrochloride, Daunorubicin Liposomal, DaunoXome, Decadron, Decitabine, Degarelix, Delta-Cortef, Deltasone, Denileukin Diftitox, Denosumab, DepoCyt, Dexamethasone, Dexamethasone Acetate, Dexamethasone Sodium Phosphate, Dexasone, Dexrazoxane, Dhad, Dic, Diodex, Docetaxel, Doxil, Doxorubicin, Doxorubicin Liposomal, Droxia, DTIC, Dtic-Dome, Duralone, Durvalumab, Eculizumab, Efudex, Ellence, Elotuzumab, Eloxatin, Elspar, Eltrombopag, Emcyt, Empliciti, Enasidenib, Enzalutamide, Epirubicin, Epoetin Alfa, Erbitux, Eribulin, Erivedge, Erleada, Erlotinib, Erwinia L-asparaginase, Estramustine, Ethyol, Etopophos, Etoposide, Etoposide Phosphate, Eulexin, Everolimus, Evista, Exemestane, Fareston, Farydak, Faslodex, Femara, Filgrastim, Firmagon, Floxuridine, Fludara, Fludarabine, Fluoroplex, Fluorouracil, Fluorouracil (cream), Fluoxymesterone, Flutamide, Folinic Acid, Folotyn, Fudr, Fulvestrant, G-Csf, Gazyva, Gefitinib, Gemcitabine, Gemtuzumab ozogamicin, Gemzar, Gilotrif, Gleevec, Gleostine, Gliadel Wafer, Gm-Csf, Goserelin, Granix, Granulocyte—Colony Stimulating Factor, Granulocyte Macrophage Colony Stimulating Factor, Halaven, Halotestin, Herceptin, Hexadrol, Hexalen, Hexamethylmelamine, Hmm, Hycamtin, Hydrea, Hydrocort Acetate, Hydrocortisone, Hydrocortisone Sodium Phosphate, Hydrocortisone Sodium Succinate, Hydrocortone Phosphate, Hydroxyurea, Ibrance, Ibritumomab, Ibritumomab Tiuxetan, Ibrutinib, Iclusig, Idamycin, Idarubicin, Idelalisib, Idhifa, Ifex, IFN-alpha, Ifosfamide, IL-11, IL-2, Imbruvica, Imatinib Mesylate, Imfinzi, Imidazole Carboxamide, Imlygic, Inlyta, Inotuzumab Ozogamicin, Interferon-Alfa, Interferon Alfa-2b (PEG Conjugate), Interleukin-2, Interleukin-11, Intron A (interferon alfa-2b), Ipilimumab, Iressa, Irinotecan, Irinotecan (Liposomal), Isotretinoin, Istodax, Ixabepilone, Ixazomib, Ixempra, Jakafi, Jevtana, Kadcyla, Keytruda, Kidrolase, Kisqali, Kymriah, Kyprolis, Lanacort, Lanreotide, Lapatinib, Lartruvo, L-Asparaginase, Lbrance, Lcr, Lenalidomide, Lenvatinib, Lenvima, Letrozole, Leucovorin, Leukeran, Leukine, Leuprolide, Leurocristine, Leustatin, Liposomal Ara-C, Liquid Pred, Lomustine, Lonsurf, L-PAM, L-Sarcolysin, Lupron, Lupron Depot, Lynparza, Marqibo, Matulane, Maxidex, Mechlorethamine, Mechlorethamine Hydrochloride, Medralone, Medrol, Megace, Megestrol, Megestrol Acetate, Mekinist, Mercaptopurine, Mesna, Mesnex, Methotrexate, Methotrexate Sodium, Methylprednisolone, Meticorten, Midostaurin, Mitomycin, Mitomycin-C, Mitoxantrone, M-Prednisol, MTC, MTX, Mustargen, Mustine, Mutamycin, Myleran, Mylocel, Mylotarg, Navelbine, Necitumumab, Nelarabine, Neosar, Neratinib, Nerlynx, Neulasta, Neumega, Neupogen, Nexavar, Nilandron, Nilotinib, Nilutamide, Ninlaro, Nipent, Niraparib, Nitrogen Mustard, Nivolumab, Nolvadex, Novantrone, Nplate, Obinutuzumab, Octreotide, Octreotide Acetate, Odomzo, Ofatumumab, Olaparib, Olaratumab, Omacetaxine, Oncospar, Oncovin, Onivyde, Ontak, Onxal, Opdivo, Oprelvekin, Orapred, Orasone, Osimertinib, Otrexup, Oxaliplatin, Paclitaxel, Paclitaxel Protein-bound, Palbociclib, Pamidronate, Panitumumab, Panobinostat, Panretin, Paraplatin, Pazopanib, Pediapred, Peg Interferon, Pegaspargase, Pegfilgrastim, Peg-Intron, PEG-L-asparaginase, Pembrolizumab, Pemetrexed, Pentostatin, Perjeta, Pertuzumab, Phenylalanine Mustard, Platinol, Platinol-AQ, Pomalidomide, Pomalyst, Ponatinib, Portrazza, Pralatrexate, Prednisolone, Prednisone, Prelone, Procarbazine, Procrit, Proleukin, Prolia, Prolifeprospan 20 with Carmustine Implant, Promacta, Provenge, Purinethol, Radium 223 Dichloride, Raloxifene, Ramucirumab, Rasuvo, Regorafenib, Revlimid, Rheumatrex, Ribociclib, Rituxan, Rituxan Hycela, Rituximab, Rituximab Hyalurodinase, Roferon-A (Interferon Alfa-2a), Romidepsin, Romiplostim, Rubex, Rubidomycin Hydrochloride, Rubraca, Rucaparib, Ruxolitinib, Rydapt, Sandostatin, Sandostatin LAR, Sargramostim, Siltuximab, Sipuleucel-T, Soliris, Solu-Cortef, Solu-Medrol, Somatuline, Sonidegib, Sorafenib, Sprycel, Sti-571, Stivarga, Streptozocin, SU11248, Sunitinib, Sutent, Sylvant, Synribo, Tafinlar, Tagrisso, Talimogene Laherparepvec, Tamoxifen, Tarceva, Targretin, Tasigna, Taxol, Taxotere, Tecentriq, Temodar, Temozolomide, Temsirolimus, Teniposide, Tespa, Thalidomide, Thalomid, TheraCys, Thioguanine, Thioguanine Tabloid, Thiophosphoamide, Thioplex, Thiotepa, Tice, Tisagenlecleucel, Toposar, Topotecan, Toremifene, Torisel, Tositumomab, Trabectedin, Trametinib, Trastuzumab, Treanda, Trelstar, Tretinoin, Trexall, Trifluridine/Tipiricil, Triptorelin pamoate, Trisenox, Tspa, T-VEC, Tykerb, Valrubicin, Valstar, Vandetanib, VCR, Vectibix, Velban, Velcade, Vemurafenib, Venclexta, Venetoclax, VePesid, Verzenio, Vesanoid, Viadur, Vidaza, Vinblastine, Vinblastine Sulfate, Vincasar Pfs, Vincristine, Vincristine Liposomal, Vinorelbine, Vinorelbine Tartrate, Vismodegib, Vlb, VM-26, Vorinostat, Votrient, VP-16, Vumon, Vyxeos, Xalkori Capsules, Xeloda, Xgeva, Xofigo, Xtandi, Yervoy, Yescarta, Yondelis, Zaltrap, Zanosar, Zarxio, Zejula, Zelboraf, Zevalin, Zinecard, Ziv-aflibercept, Zoladex, Zoledronic Acid, Zolinza, Zometa, Zydelig, Zykadia, Zytiga, or any combination thereof.

The terms “effective amount” and “therapeutically effective amount” of an agent or compound are used in the broadest sense to refer to a nontoxic but sufficient amount of an active agent or compound to provide the desired effect or benefit.

The term “benefit” is used in the broadest sense and refers to any desirable effect and specifically includes clinical benefit as defined herein. Clinical benefit can be measured by assessing various endpoints, e.g., inhibition, to some extent, of disease progression, including slowing down and complete arrest; reduction in the number of disease episodes and/or symptoms; reduction in lesion size; inhibition (i.e., reduction, slowing down or complete stopping) of disease cell infiltration into adjacent peripheral organs and/or tissues; inhibition (i.e. reduction, slowing down or complete stopping) of disease spread; decrease of auto-immune response, which may, but does not have to, result in the regression or ablation of the disease lesion; relief, to some extent, of one or more symptoms associated with the disorder; increase in the length of disease-free presentation following treatment, e.g., progression-free survival; increased overall survival; higher response rate; and/or decreased mortality at a given point of time following treatment.

The terms “cancer” and “cancerous” refer to or describe the physiological condition in mammals that is typically characterized by unregulated cell growth. Included in this definition are benign and malignant cancers. Examples of cancer include but are not limited to, carcinoma, lymphoma, blastoma, sarcoma, and leukemia. More particular examples of such cancers include adrenocortical carcinoma, bladder urothelial carcinoma, breast invasive carcinoma, cervical squamous cell carcinoma, endocervical adenocarcinoma, cholangiocarcinoma, colon adenocarcinoma, lymphoid neoplasm diffuse large B-cell lymphoma, esophageal carcinoma, glioblastoma multiforme, head and neck squamous cell carcinoma, kidney chromophobe, kidney renal clear cell carcinoma, kidney renal papillary cell carcinoma, acute myeloid leukemia, brain lower grade glioma, liver hepatocellular carcinoma, lung adenocarcinoma, lung squamous cell carcinoma, mesothelioma, ovarian serous cystadenocarcinoma, pancreatic adenocarcinoma, pheochromocytoma, paraganglioma, prostate adenocarcinoma, rectum adenocarcinoma, sarcoma, skin cutaneous melanoma, stomach adenocarcinoma, testicular germ cell tumors, thyroid carcinoma, thymoma, uterine carcinosarcoma, uveal melanoma. Other examples include breast cancer, lung cancer, lymphoma, melanoma, liver cancer, colorectal cancer, ovarian cancer, bladder cancer, renal cancer or gastric cancer. Further examples of cancer include neuroendocrine cancer, non-small cell lung cancer (NSCLC), small cell lung cancer, thyroid cancer, endometrial cancer, biliary cancer, esophageal cancer, anal cancer, salivary, cancer, vulvar cancer or cervical cancer.

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein.

As used herein, the term “about” when used in conjunction with numerical values and/or ranges generally refers to those numerical values and/or ranges near to a recited numerical value and/or range. In some instances, the term “about” can mean within +10% of the recited value. For example, in some instances, “about 100 [units]” can mean within +10% of 100 (e.g., from 90 to 110).

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1. Derivation of a 38-Marker Gene Panel

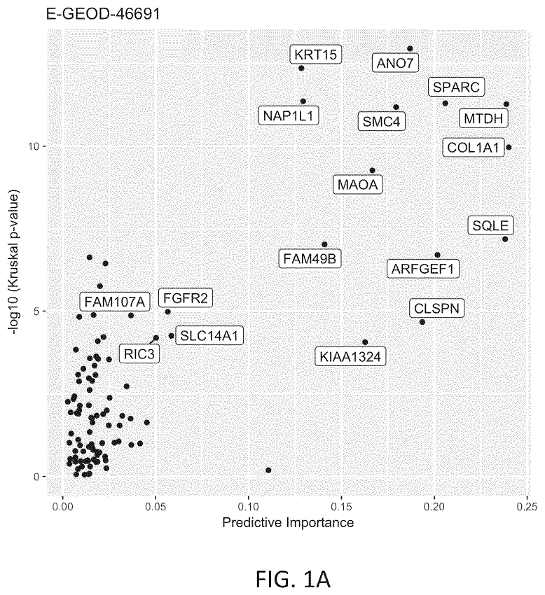

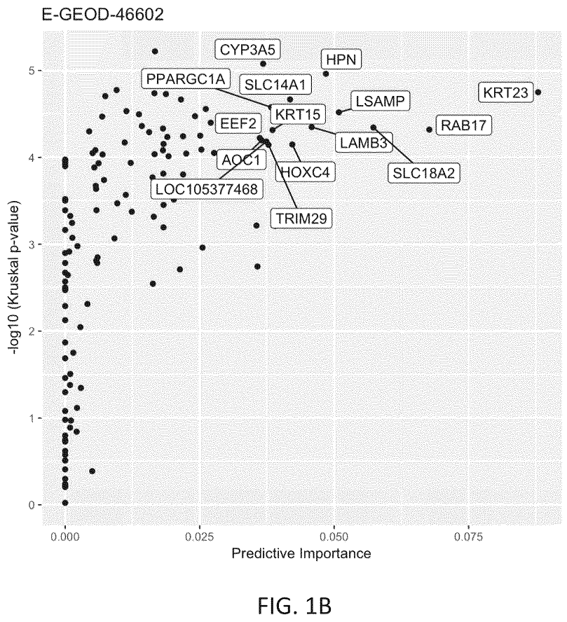

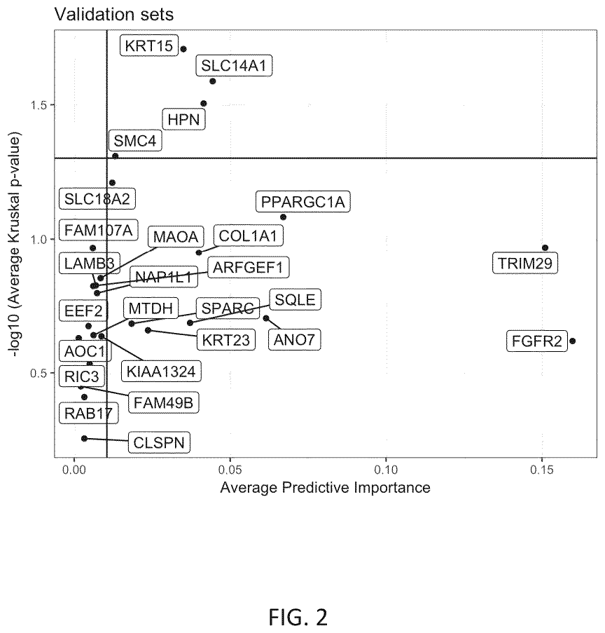

Two microarray datasets (E-GEOD-46691 and E-GEOD-46602, Table 2) were used as derivation cohorts (n=595 samples). Random Forest algorithm was applied to each dataset to identify the most important sets of transcripts that are predictive of phenotypic diversity within each set. Each microarray dataset comprised 22,011 and 54,675 probe sets, respectively. The Random Forest-drive marker selection algorithm identified n=129 transcripts as predictive of disease progression across the two datasets. Of these n=30 exhibited high Predictive Importance scores in both datasets (FIGS. 1A-1B). Three microarray datasets (E-GEOD-62116, E-GEOD-62667, E-GEOD-72220, Table 2) were then used to validate the putative Prostate Cancer signature. For each transcript across the validation cohort (n=564 samples) Predictive Importance and Kruskal-Wallis p-values were obtained and averaged across the three datasets (FIG. 2). Examination of the literature identified the ARv7 variant, the ERG-TRMS22 fusion gene and AR1/AR2 signaling as additional gene sets to include and evaluate.

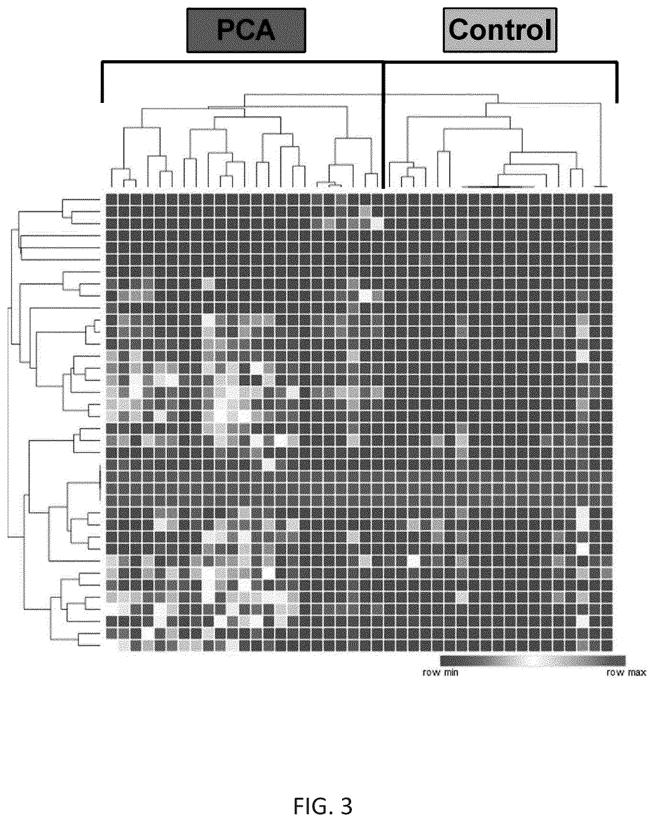

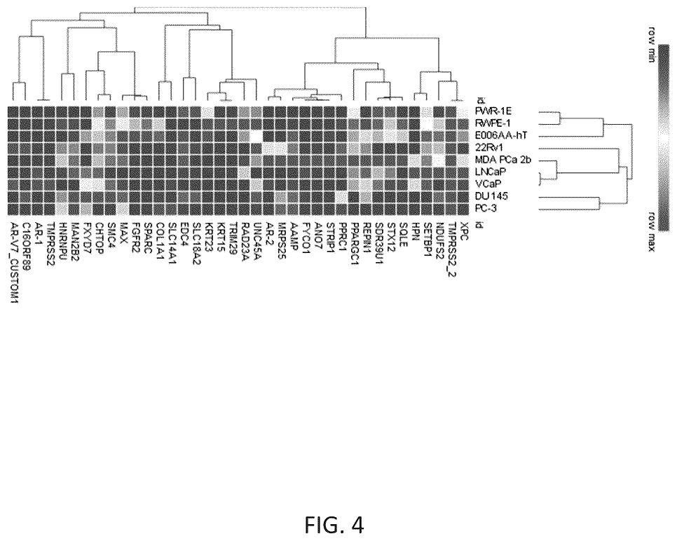

Evaluation of transcripts in a preliminary dataset of blood samples from prostate cancer (n=20) and matched normal blood (n=20) confirmed expression of the 37 genes as markers of PCA (Table 3). These genes were demonstrated to be highly expressed in PCA tumor tissue compared to normal prostate and this could be used to effectively differentiate tumor from control (FIG. 3). They also differentiated seven different PCA cell lines, 22Rv1 and E006AA-hT (localized); VCaP; PC-3; LNCaP; DU145 and MDA PCa2b (all metastatic) from two normal prostate epithelial lines: PWR-1E and RWPE-1 (FIG. 4). These data demonstrate the candidate target transcripts are produced by neoplastic transformed prostate epithelial cell and are detectable in blood.

An artificial intelligence model of prostate cancer disease was built using normalized gene expression of these 37 markers in whole blood from Controls (n=100), and PCA (n=21) samples. The dataset was randomly split into training and testing partitions for model creation and validation respectively. Twelve algorithms were evaluated (XGB, RF, glmnet, cforest, CART, treebag, knn, nnet, SVM-radial, SVM-linear, NB and mlp). The top performing algorithm (XGB—“gradient boosting”) best predicted the training data. In the test set, XGB produced probability scores that predicted the sample. Each probability score reflects the “certainty” of an algorithm that an unknown sample belongs to either “Control” or “PCA” class. For example, an unknown sample Si can have the following probability vector [Control=20%, PCA=80%]. This sample would be considered a PCA sample.

TABLE 2
Summary of all public microarray datasets that were used to
derived Prostate Cancer specific gene signature.

| Accession Number | Sample Count | Dataset Description |
|---|---|---|
| E-GEOD-46691 | 545 | Gleason Score 5-10 |
| | | Score 5 (n = 3) |
| | | Score 6 (n = 60) |
| | | Score 7 (n = 271) |
| | | Score 8 (n = 68) |
| | | Score 9 (n = 134) |
| | | Score 10 (n = 9) |
| E-GEOD-46602 | 50 | Benign Prostate Glands (n = 14) |
| | | Prostate Tumor (n = 36) |
| E-GEOD-62116 | 235 | Gleason Score 5-10 |
| | | Score 5 (n = 1) |
| | | Score 6 (n = 17) |
| | | Score 7 (n = 119) |
| | | Score 8 (n = 39) |
| | | Score 9 (n = 58) |
| | | Score 10 (n = 1) |
| E-GEOD-62667 | 182 | Gleason Score 6-9 |
| | | Score 6 (n = 25) |
| | | Score 7 (n = 112) |
| | | Score 8 (n = 23) |
| | | Score 9 (n = 22) |
| E-GEOD-72220 | 147 | Normal Tissue (n = 90) |
| | | Tumor Tissue (n = 57 |

TABLE 3
37 PCA marker gene panel (not including the housekeeping gene)

| | | NCBI | | | | | |
|---|---|---|---|---|---|---|---|
| Prostate Cancer Biomarker Gene | | Chromosome | UniGene | | Exon | Assay | Amplicon |
| Symbol | Name | Location | ID | RefSeq | Boundary | Location | Length |
| AAMP | Angio-associated | Chr.2: | Hs.83347 | NM_001087.4 | 2-3 | 395 | 58 |
| | migratory cell | 218264129- | | | | | |
| | protein | 218270209 | | | | | |
| ANO7 | Anoctamin 7 | Chr.2: | Hs.163909 | NM_001001891.3 | 5-6 | 686 | 60 |
| | | 241188509- | | | | | |
| | | 241239602 | | | | | |
| AR | androgen receptor | Chr.X: | Hs.76704 | NM_000044.3 | 2-3 | 2882 | 144 |
| | | 67544032- | | | | | |
| | | 67730619 | | | | | |
| AR-V7 | androgen receptor | AR truncation | | | | | 73 |
| | transcript variant 7 | | | | | | |
| C16orf89 | chromosome 16 | Chr.16: | Hs.11782 | NM_001098514.2 | 5-6 | 995 | 75 |
| | open reading frame | 5042772- | | | | | |
| | 89 | 5066145 | | | | | |
| CHTOP | chromatin target of | Chr.1: | Hs.611057 | NM_001206612.1 | 2-3 | 445 | 66 |
| | PRMT1 | 153633982- | | | | | |
| | | 153646306 | | | | | |
| COL1A1 | collagen type I alpha | Chr.17: | Hs.172928 | NM_000088.3 | 1-2 | 230 | 66 |
| | 1 | 50184096- | | | | | |
| | | 50201648 | | | | | |
| EDC4 | enhancer of mRNA | Chr.16: | Hs.75682 | NM_014329.4 | 27-28 | 4089 | 72 |
| | decapping 4 | 67873023- | | | | | |
| | | 67884514 | | | | | |
| FGFR2 | fibroblast growth | Chr.10: | Hs.533683 | NM_000141.4 | 13-14 | 2516 | 74 |
| | factor receptor 2 | 121478330- | | | | | |
| | | 121598656 | | | | | |
| FXYD7 | FXYD domain | Chr.19: | Hs.134729 | NM_022006.1 | 5-6 | 300 | 89 |
| | containing ion | 35143250- | | | | | |
| | transport regulator 7 | 35154302 | | | | | |
| FYCO1 | FYVE and coiled- | Chr.3: | Hs.200227 | NM_024513.3 | 11-12 | 3651 | 59 |
| | coil domain | 45917899- | | | | | |
| | containing 1 | 45995824 | | | | | |
| HNRNPU | heterogeneous | Chr.1: | Hs.106212 | NM_004501.3 | 8-9 | 1773 | 79 |
| | nuclear | 244842123- | | | | | |
| | ribonucleoprotein U | 244864720 | | | | | |
| HPN | hepsin | Chr.19: | Hs.182385 | NM_002151.2 | 10-11 | 1052 | 89 |
| | | 35040506- | | | | | |
| | | 35066573 | | | | | |
| KRT15 | keratin 15 | Chr.17: | Hs.654570 | NM_002275.3 | 7-8 | 1461 | 81 |
| | | 41513745- | | | | | |
| | | 41522413 | | | | | |
| KRT23 | keratin 23 | Chr.17: | Hs.9029 | NM_001282433.1 | 5-6 | 1045 | 90 |
| | | 40922696- | | | | | |
| | | 40937643 | | | | | |
| MAN2B2 | mannosidase alpha | Chr.4: | Hs.188464 | NM_001292038.1 | 5-6 | 716 | 55 |
| | class 2B member 2 | 6575174- | | | | | |
| | | 6622403 | | | | | |
| MAX | MYC associated | Chr.14: | Hs.285354 | NM_001320415.1 | 3-4 | 377 | 61 |
| | factor X | 65006101- | | | | | |
| | | 65102695 | | | | | |
| MRPS25 | mitochondrial | Chr.3: | Hs.657764 | NM_022497.4 | 2-3 | 380 | 80 |
| | ribosomal protein | 15042251- | | | | | |
| | S25 | 15065337 | | | | | |
| NDUFS2 | NADH: ubiquinone | Chr.1 | Hs.173611 | NM_001166159.1 | 3-4 | 632 | 80 |
| | oxidoreductase core | 161197377- | | | | | |
| | subunit S2 | 161214395 | | | | | |
| PPARGC1A | PPARG coactivator | Chr.4: | Hs.527078 | NM_013261.3 | 7-8 | 998 | 83 |
| | 1 alpha | 23792021- | | | | | |
| | | 24472975 | | | | | |
| PPRC1 | peroxisome | Chr.10: | Hs.533551 | NM_001288727.1 | 1-2 | 224 | 55 |
| | proliferator- | 102132994- | | | | | |
| | activated receptor | 102150333 | | | | | |
| | gamma, coactivator- | | | | | | |
| | related 1 | | | | | | |
| RAD23A | RAD23 homolog A, | Chr.19: | Hs.643267 | NM_001270362.1 | 1-2 | 203 | 74 |
| | nucleotide excision | 12945814- | | | | | |
| | repair protein | 12953643 | | | | | |
| REPIN1 | replication initiator | Chr.7: | Hs.647086 | NM_013400.3 | 3-4 | 474 | 70 |
| | 1 | 150368228- | | | | | |
| | | 150374044 | | | | | |
| SDR39U1 | short chain | Chr.14: | Hs.643552 | NM_020195.2 | 4-5 | 358 | 91 |
| | dehydrogenase/ | 24439766- | | | | | |
| | reductase family | 24442905 | | | | | |
| | 39U member 1 | | | | | | |
| SETBP1 | SET binding protein | Chr.18: | Hs.435458 | NM_001130110.1 | 2-3 | 882 | 70 |
| | 1 | 44680173- | | | | | |
| | | 45068510 | | | | | |
| SLC14A1 | solute carrier family | Chr.18: | Hs.101307 | NM_001128588.3 | 7-8 | 1057 | 80 |
| | 14 member 1 (Kidd | 45724123- | | | | | |
| | blood group) | 45752520 | | | | | |
| SLC18A2 | solute carrier family | Chr.10: | Hs.596992 | NM_003054.4 | 15-16 | 1605 | 145 |
| | 18 member A2 | 117241073- | | | | | |
| | | 117279430 | | | | | |
| SMC4 | structural | Chr.3: | Hs.58992 | NM_001002800.2 | 5-6 | 1134 | 91 |
| | maintenance of | 160399304- | | | | | |
| | chromosomes 4 | 160434962 | | | | | |
| SPARC | secreted protein | Chr.5: | Hs.111779 | NM_001309443.1 | 6-7 | 650 | 76 |
| | acidic and cysteine | 151661096- | | | | | |
| | rich | 151687054 | | | | | |
| SQLE | squalene epoxidase | Chr.8: | Hs.71465 | NM_003129.3 | 9-10 | 2368 | 109 |
| | | 124998478- | | | | | |
| | | 125022283 | | | | | |
| STRIP1 | striatin interacting | Chr.1: | Hs.584996 | NM_001270768.1 | 12-13 | 1303 | 64 |
| | protein 1 | 110031577- | | | | | |
| | | 110054641 | | | | | |
| STX12 | syntaxin 12 | Chr.1: | Hs.523855 | NM_177424.2 | 1-2 | 248 | 70 |
| | | 27773183- | | | | | |
| | | 27824452 | | | | | |
| TMPRSS2_1 | TMPRSS2-ERG | TMPRSS2 | | DQ204772.1 | | 49 | 106 |
| | fusion | exon 1 and | | | | | |
| TMPRSS2_2 | TMPRSS2-ERG | ERG exon 4 | | OM_TMPRSS2- | | 58 | 88 |
| | fusion | fusion | | ERG_T2E4_COSF28.0 | | | |
| TRIM29 | tripartite motif | Chr.11: | Hs.504115 | NM_012101.3 | 2-3 | 1025 | 62 |
| | containing 29 | 120111286- | | | | | |
| | | 120138179 | | | | | |
| UNC45A | unc-45 myosin | Chr.15: | Hs.389461 | NM_001039675.1 | 20-21 | 3095 | 62 |
| | chaperone A | 90929980- | | | | | |
| | | 90954093 | | | | | |
| XPC | XPC complex | Chr.3: | Hs.475538 | NM_004628.4 | 2-3 | 403 | 104 |
| | subunit, DNA | 14145147- | | | | | |
| | damage recognition | 14178672 | | | | | |
| | and repair factor | | | | | | |

### Example 2. Clinical Utility

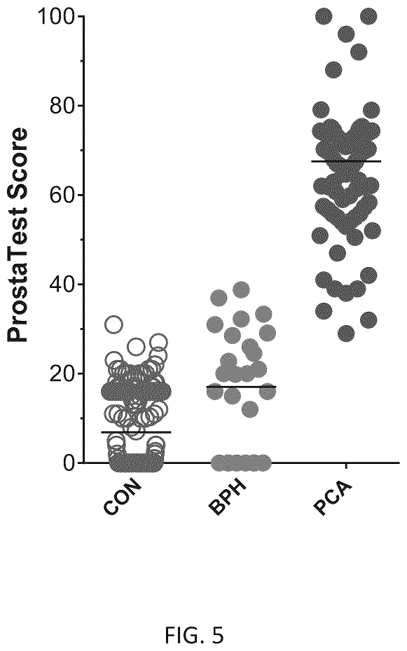

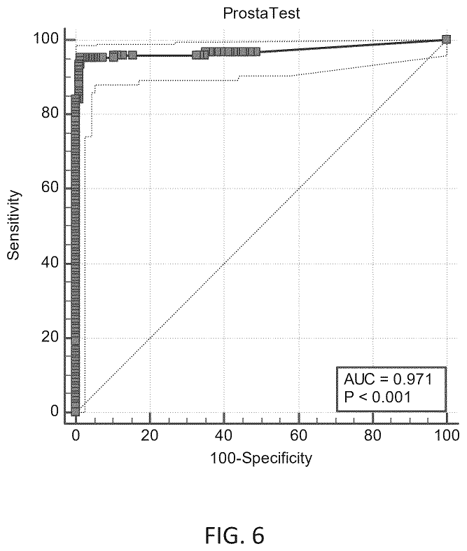

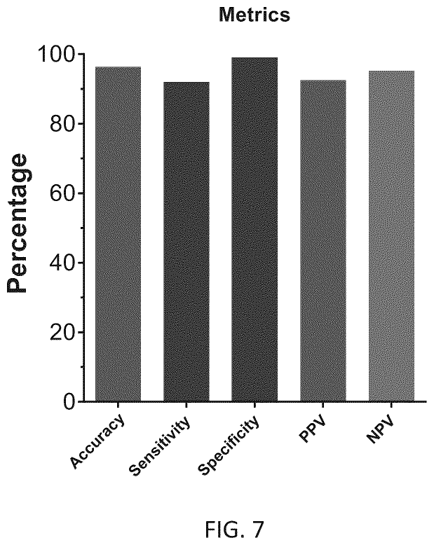

The ProstaTest scores were significantly (p<0.001) elevated in PCA (63±19%) compared to men with benign prostate hyperplasia (17±13%) and controls (8±9%) (FIG. 5). There was no difference in levels between controls and hyperplasia. The data (receiver operator curve analysis and metrics) for the utility of the test to differentiate patients with prostate cancer (n=125) from controls (n=201) in the validation is included in FIG. 6. The score exhibited an area under the curve (AUROC) of 0.97. The metrics are: sensitivity: 92% and specificity: 99% (FIG. 7). The Youden index J is 0.94 and the Z-statistic for differentiating controls was 38.9.

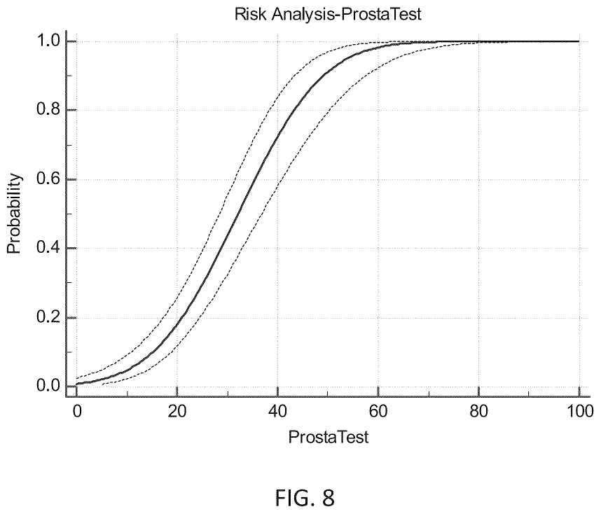

A Probit-risk assessment plot identified a ProstaTest score >30 was 50% accurate for predicting PCA in a blood sample (FIG. 8). This was increased to 60% at a ProstaTest score >32 and was >80% at a score >34. The tool can therefore accurately differentiate between controls and prostate cancer disease.

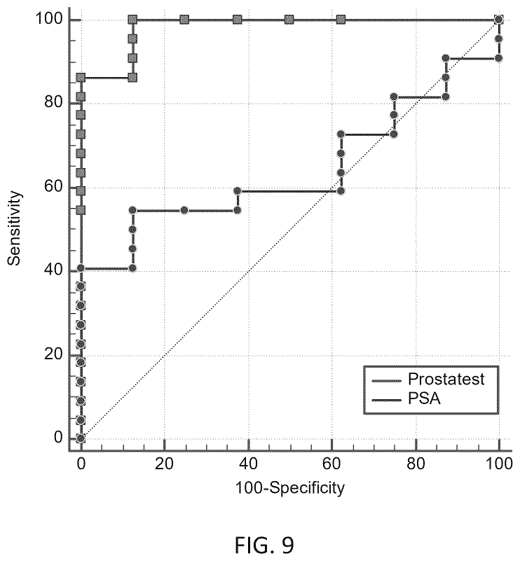

The ProstaTest scores were significantly (p<0.001) elevated in high grade (Gleason score ≥7: 70±19%) compared to low grade (Gleason 5±6) PCA (41±7%). The data (receiver operator curve analysis and metrics) for the utility of the test to differentiate high from low grade scores is included in FIG. 9. The score exhibited an area under the curve (AUROC) of 0.98. The metrics are: sensitivity: 100% and specificity: 88%. The Youden index J is 0.87. PSA levels, in comparison, exhibited an AUROC of 64%. The sensitivity and specificity were 54% and 87% respectively. The Z-statistic for differentiating the ProstaTest from PSA was 3.05 (p=0.002).

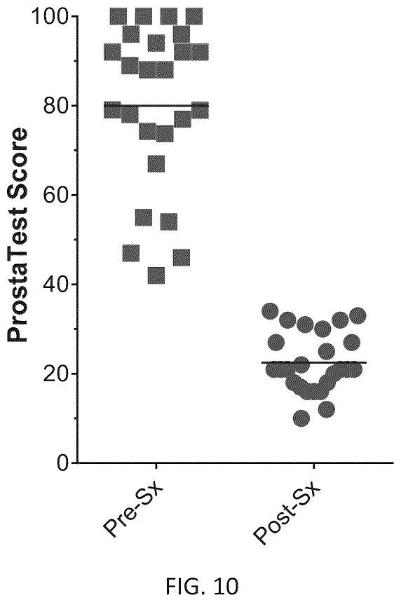

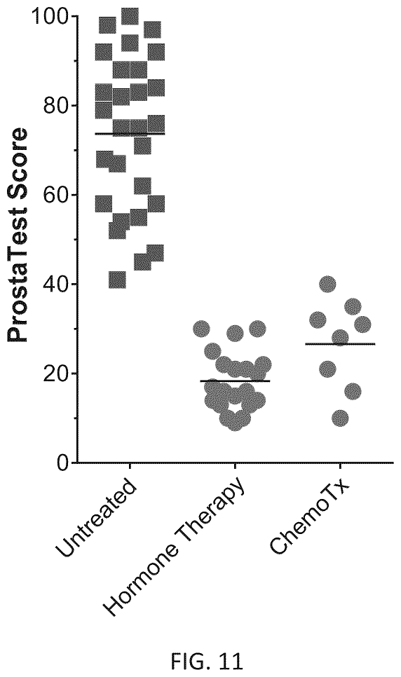

Specific evaluation of a PCA cohort before and after surgery identified that complete removal of a tumor and no evidence of disease was associated with a significant decrease (p<0.0001) in the ProstaTest (FIG. 10). Levels were not significantly different to controls or those with prostate hyperplasia. Evaluation of separate cohort identified that patients who underwent and responded to therapy exhibited a significant lower score (p<0.001) that those diagnosed with disease (FIG. 11). Therapies included hormone and chemotherapy. The tool can therefore accurately identify treatment response in prostate cancer disease.

### REFERENCES

Ferlay J, Steliarova-Foucher E, Lortet-Tieulent J, Rosso S, Coebergh J W, Comber H, Forman D, Bray F. Cancer incidence and mortality patterns in Europe: estimates for 40 countries in 2012. Eur J Cancer. 2013; 49: 1374-403. doi: 10.016/j.ejca.2012.12.027. Epub 3 Feb. 26.
Jemal A, Fedewa S A, Ma J, Siegel R, Lin C C, Brawley O, Ward E M. Prostate Cancer Incidence and PSA Testing Patterns in Relation to USPSTF Screening Recommendations. JAMA. 2015; 314: 2054-61. doi: 10.1001/jama.2015.14905.
Nam R K, Satkunavisam R, Chin J L, Izawa J, Trachtenberg J, Rendon R, Bell D, Singal R, Sherman C, Sugar L, Chagin K, Kattan W M. Next-generation prostate cancer risk calculator for primary care physicians. Can Urol Assoc J. 2017; 1.
The Molecular Taxonomy of Primary Prostate Cancer. Cell. 2015; 163: 1011-25. doi: 10.6/j.cell.2015.10.025.
Lalonde E, Ishkanian A S, Sykes J, Fraser M, Ross-Adams H, Erho N, Dunning M J, Halim S, Lamb A D, Moon N C, Zafarana G, Warren A Y, Meng X, et al. Tumour genomic and microenvironmental heterogeneity for integrated prediction of 5-year biochemical recurrence of prostate cancer: a retrospective cohort study. Lancet Oncol. 2014; 15: 1521-32. doi: 10.016/S470-2045(14)71021-6. Epub 2014 Nov. 13.
Barbieri C E, Baca S C, Lawrence M S, Demichelis F, Blattner M, Theurillat J P, White T A, Stojanov P, Van Allen E, Stransky N, Nickerson E, Chae S S, Boysen G, et al. Exome sequencing identifies recurrent SPOP, FOXA1 and MED12 mutations in prostate cancer. Nat Genet. 2012; 44: 685-9. doi: 10.1038/ng.2279.
Tomlins S A, Rhodes D R, Perner S, Dhanasekaran S M, Mehra R, Sun X W, Varambally S, Cao X, Tchinda J, Kuefer R, Lee C, Montie J E, Shah R B, et al. Recurrent fusion of TMPRSS2 and ETS transcription factor genes in prostate cancer. Science. 2005; 310: 644-8. doi: 10.1126/science.1117679.
Pettersson A, Graff R E, Bauer S R, Pitt M J, Lis R T, Stack E C, Martin N E, Kunz L, Penney K L, Ligon A H, Suppan C, Flavin R, Sesso H D, et al. The TMPRSS2:ERG rearrangement, ERG expression, and prostate cancer outcomes: a cohort study and meta-analysis. Cancer Epidemiol Biomarkers Prev. 2012; 21: 1497-509. doi: 10.158/055-9965.EPI-12-0042. Epub 2012 Jun. 26.
Antonarakis E S, Lu C, Luber B, Wang H, Chen Y, Nakazawa M, Nadal R, Paller C J, Denmeade S R, Carducci M A, Eisenberger M A, Luo J. Androgen Receptor Splice Variant 7 and Efficacy of Taxane Chemotherapy in Patients With Metastatic Castration-Resistant Prostate Cancer. JAMA Oncol. 2015; 1: 582-91. doi: 10.1001/jamaoncol.2015.1341.
Velonas V M, Woo H H, dos Remedios C G, Assinder S J. Current status of biomarkers for prostate cancer. Int J Mol Sci. 2013; 14: 11034-60. doi: 10.3390/ijms140611034.
Schroder F H, Hugosson J, Roobol M J, Tammela T L, Zappa M, Nelen V, Kwiatkowski M, Lujan M, Maattanen L, Lilja H, Denis L J, Recker F, Paez A, et al. Screening and prostate cancer mortality: results of the European Randomised Study of Screening for Prostate Cancer (ERSPC) at 13 years of follow-up. Lancet. 2014; 384: 2027-35. doi: 10.1016/S0140-6736(14)60525-0. Epub 2014 Aug. 6.
Schroder F H, Carter H B, Wolters T, van den Bergh R C, Gosselaar C, Bangma C H, Roobol M J. Early detection of prostate cancer in 2007. Part 1: PSA and PSA kinetics. Eur Urol. 2008; 53: 468-77. doi: 10.1016/j.eururo.2007.10.047. Epub November 5.
Cooperberg M R, Pasta D J, Elkin E P, Litwin M S, Latini D M, Du Chane J, Carroll P R. The University of California, San Francisco Cancer of the Prostate Risk Assessment score: a straightforward and reliable preoperative predictor of disease recurrence after radical prostatectomy. J Urol. 2005; 173: 1938-42. doi: 10.097/01.ju.0000158155.33890.e7. Troyer D A, Lucia M S, de Bruine A P, Mendez-Meza R, Baldewijns M M, Dunscomb N,
Van Engeland M, McAskill T, Bierau K, Louwagie J, Bigley J W. Prostate cancer detected by methylated gene markers in histopathologically cancer-negative tissues from men with subsequent positive biopsies. Cancer Epidemiol Biomarkers Prev. 2009; 18: 2717-22. doi: 10.1158/055-9965.EPI-09-0068. Epub 2009 Sep. 15.
Van Neste L, Partin A W, Stewart G D, Epstein J I, Harrison D J, Van Criekinge W. Risk score predicts high-grade prostate cancer in DNA-methylation positive, histopathologically negative biopsies. Prostate. 2016; 76: 1078-87. doi: 10.02/pros.23191. Epub 2016 Apr. 28.
Stewart G D, Van Neste L, Delvenne P, Delree P, Delga A, McNeill S A, O'Donnell M, Clark J, Van Criekinge W, Bigley J, Harrison D J. Clinical utility of an epigenetic assay to detect occult prostate cancer in histopathologically negative biopsies: results of the MATLOC study. J Urol. 2013; 189: 1110-6. doi: 10.016/j.juro.2012.08.219. Epub October 8.
Day J R, Jost M, Reynolds M A, Groskopf J, Rittenhouse H. PCA3: from basic molecular science to the clinical lab. Cancer Lett. 2011; 301: 1-6. doi: 10.1016/j.canlet.2010.10.019. Epub November 18.
Cuzick J, Swanson G P, Fisher G, Brothman A R, Berney D M, Reid J E, Mesher D, Speights V O, Stankiewicz E, Foster C S, Moller H, Scardino P, Warren J D, et al. Prognostic value of an RNA expression signature derived from cell cycle proliferation genes in patients with prostate cancer: a retrospective study. Lancet Oncol. 2011; 12: 245-55. doi: 10.1016/S470-2045(10)70295-3.
Erho N, Crisan A, Vergara I A, Mitra A P, Ghadessi M, Buerki C, Bergstralh E J, Kollmeyer T, Fink S, Haddad Z, Zimmermann B, Sierocinski T, Ballman K V, et al. Discovery and validation of a prostate cancer genomic classifier that predicts early metastasis following radical prostatectomy. PLoS One. 2013; 8: e66855. doi: 10.1371/journal.pone.0066855. Print 2013.
Karnes R J, Bergstralh E J, Davicioni E, Ghadessi M, Buerki C, Mitra A P, Crisan A, Erho N, Vergara I A, Lam L L, Carlson R, Thompson D J, Haddad Z, et al. Validation of a genomic classifier that predicts metastasis following radical prostatectomy in an at risk patient population. J Urol. 2013; 190: 2047-53. doi: 10.1016/j.juro.2013.06.017. Epub June 11.
Knezevic D, Goddard A D, Natraj N, Cherbavaz D B, Clark-Langone K M, Snable J, Watson D, Falzarano S M, Magi-Galluzzi C, Klein E A, Quale C. Analytical validation of the Oncotype D X prostate cancer assay—a clinical R T-PCR assay optimized for prostate needle biopsies. BMC Genomics. 2013; 14:690: 10.1186/471-2164-14-690.
Klein E A, Cooperberg M R, Magi-Galluzzi C, Simko J P, Falzarano S M, Maddala T, Chan J M, Li J, Cowan J E, Tsiatis A C, Cherbavaz D B, Pelham R J, Tenggara-Hunter I, et al. A 17-gene assay to predict prostate cancer aggressiveness in the context of Gleason grade heterogeneity, tumor multifocality, and biopsy undersampling. Eur Urol. 2014; 66: 550-60. doi: 10.1016/j.eururo.2014.05.004. Epub May 16.
Zhao S G, Evans J R, Kothari V, Sun G, Larm A, Mondine V, Schaeffer E M, Ross A E, Klein E A, Den R B, Dicker A P, Karnes R J, Erho N, et al. The Landscape of Prognostic Outlier Genes in High-Risk Prostate Cancer. Clin Cancer Res. 2016; 22: 1777-86. doi: 10.158/078-0432.CCR-15-1250. Epub 2015 Dec. 2.
Zhao S G, Chang S L, Spratt D E, Erho N, Yu M, Ashab H A, Alshalalfa M, Speers C, Tomlins S A, Davicioni E, Dicker A P, Carroll P R, Cooperberg M R, et al. Development and validation of a 24-gene predictor of response to postoperative radiotherapy in prostate cancer: a matched, retrospective analysis. Lancet Oncol. 2016; 17: 1612-20. doi: 10.016/S470-2045(16)30491-0. Epub 2016 Oct. 12.
Ross R W, Galsky M D, Scher H I, Magidson J, Wassmann K, Lee G S, Katz L, Subudhi S K, Anand A, Fleisher M, Kantoff P W, Oh W K. A whole-blood RNA transcript-based prognostic model in men with castration-resistant prostate cancer: a prospective study. Lancet Oncol. 2012; 13: 1105-13. doi: 10.016/S470-2045(12)70263-2. Epub 2012 Oct. 9.
Olmos D, Brewer D, Clark J, Danila D C, Parker C, Attard G, Fleisher M, Reid A H, Castro E, Sandhu S K, Barwell L, Oommen N B, Carreira S, et al. Prognostic value of blood mRNA expression signatures in castration-resistant prostate cancer: a prospective, two-stage study. Lancet Oncol. 2012; 13: 1114-24. doi: 10.016/S470-2045(12)70372-8. Epub 2012 Oct. 9.
Danila D C, Anand A, Schultz N, Heller G, Wan M, Sung C C, Dai C, Khanin R, Fleisher M, Lilja H, Scher H I. Analytic and clinical validation of a prostate cancer-enhanced messenger RNA detection assay in whole blood as a prognostic biomarker for survival. Eur Urol. 2014; 65: 1191-7. doi: 10.016/j.eururo.2013.07.006. Epub July 26.
Wang L, Gong Y, Chippada-Venkata U, Heck M M, Retz M, Nawroth R, Galsky M, Tsao C K, Schadt E, de Bono J, Olmos D, Zhu J, Oh W K. A robust blood gene expression-based prognostic model for castration-resistant prostate cancer. BMC Med. 2015; 13:201: 10.1186/s12916-015-0442-0.
Kohli M, Young C Y, Tindall D J, Nandy D, McKenzie K M, Bevan G H, Donkena K V. Whole blood defensin mRNA expression is a predictive biomarker of docetaxel response in castration-resistant prostate cancer. Onco Targets Ther. 2015; 8:1915-22: 10.2147/OTT.S86637. eCollection 2015.
Diaz-Uriarte R, Alvarez de Andres S. Gene selection and classification of microarray data using random forest. BMC Bioinformatics. 2006; 7: 3. doi: 10.1186/1471-2105-7-3.

### EQUIVALENTS

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

What is claimed is:

1. A method for detecting a prostate cancer in a subject in need thereof, the method comprising:
determining the expression level of at least 38 biomarkers from a test sample from the subject by contacting the test sample with a plurality of agents specific to detect the expression of the at least 38 biomarkers, wherein the at least 38 biomarkers comprise AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, XPC, and a housekeeping gene;
normalizing the expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC to the expression level of the housekeeping gene, thereby obtaining a normalized expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC;
inputting each normalized expression level into an algorithm to generate a score;
comparing the score with a first predetermined cutoff value; and
producing a report, wherein the report identifies the presence of a prostate cancer in the subject when the score is equal to or greater than the first predetermined cutoff value or identifies the absence of a prostate cancer in the subject when the score is less than the first predetermined cutoff value.

2. A method for evaluating the response of a subject having a prostate cancer to a first therapy, the method comprising:
(1) at a first time point:
(a) determining the expression level of at least 38 biomarkers from a test sample from the subject by contacting the test sample with a plurality of agents specific to detect the expression of the at least 38 biomarkers, wherein the at least 38 biomarkers comprise AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, XPC, and a housekeeping gene;
(b) normalizing the expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC to the expression level of the housekeeping gene, thereby obtaining a normalized expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC;
(c) inputting each normalized expression level into an algorithm to generate a first score;
(2) at a second time point, wherein the second time point is after the first time point and after the administration of the first therapy to the subject:
(d) determining the expression level of the at least 38 biomarkers from a test sample from the subject by contacting the test sample with a plurality of agents specific to detect the expression of the at least 38 biomarkers;
(e) normalizing the expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC to the expression level of the housekeeping gene, thereby obtaining a normalized expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC;
(f) inputting each normalized expression level into an algorithm to generate a second score;
(3) comparing the first score with the second score; and
(4) producing a report, wherein the report identifies that the subject is responsive to the first therapy when the second score is significantly decreased as compared to the first score or identifies that the subject is not responsive to the first therapy when the second score is not significantly decreased as compared to the first score.

3. A method of treating prostate cancer in a subject comprising:
determining the expression level of at least 38 biomarkers from a test sample from the subject by contacting the test sample with a plurality of agents specific to detect the expression of the at least 38 biomarkers, wherein the at least 38 biomarkers comprise AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, XPC, and a housekeeping gene, wherein the housekeeping gene is TOX4;
normalizing the expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC to the expression level of the housekeeping gene, thereby obtaining a normalized expression level of each of AAMP, ANO7, AR, AR-V7, C16orf89, CHTOP, COL1A1, EDC4, FGFR2, FXYD7, FYCO1, HNRNPU, HPN, KRT15, KRT23, MAN2B2, MAX, MRPS25, NDUFS2, PPARGC1A, PPRC1, RAD23A, REPIN1, SDR39U1, SETBP1, SLC14A1, SLC18A2, SMC4, SPARC, SQLE, STRIP1, STX12, TMPRSS2_1, TMPRSS2_2, TRIM29, UNC45A, and XPC;
inputting each normalized expression level into an algorithm to generate a score, wherein the algorithm is a product of a model of prostate cancer derived using the XGB algorithm;
comparing the score with a first predetermined cutoff value;
determining that the score is equal to or greater than the predetermined cutoff value, thereby identifying the subject as having prostate cancer; and
administering to the subject identified as having prostate cancer a therapy, wherein the therapy comprises radiation therapy, cryotherapy, hormone therapy, chemotherapy, vaccine treatment, bone-directed treatment, or a combination thereof.

4. The method of claim 3, wherein the first predetermined cutoff value is at least 33% on a scale of 0-100%.

5. The method of claim 3, wherein the predetermined cutoff value has a sensitivity of identifying the subject as having prostate cancer that is-at least 92%.

6. The method of claim 3, wherein the predetermined cutoff value has a specificity of identifying the subject as having prostate cancer that is at least 95%.

7. The method of claim 3, wherein at least one of the at least 38 biomarkers is RNA, cDNA, or protein.

8. The method of claim 7, wherein when the biomarker is RNA, the RNA is reverse transcribed to produce cDNA, and the produced cDNA expression level is detected.

9. The method of claim 3, wherein the first predetermined cutoff value is derived from a plurality of reference samples obtained from subjects not having or not diagnosed with a prostate cancer.

10. The method of claim 3, wherein the test sample comprises blood, serum, plasma, or neoplastic tissue.

11. The method of claim 10, wherein reference sample comprises blood, serum, plasma, or non-neoplastic tissue.

12. The method of claim 3, wherein the therapy comprises hormone therapy, wherein the hormone therapy comprises androgen suppression therapy.

13. The method of claim 3, wherein the therapy comprises chemotherapy, wherein the chemotherapy comprises docetaxel, cabazitaxel, mitoxantrone, estramustine, or a combination thereof.

14. The method of claim 3, wherein the therapy comprises vaccine treatment, wherein the vaccine treatment comprises Sipuleucel-T.

15. The method of claim 3, wherein the therapy comprises bone-directed treatment, wherein the bone-directed treatment comprises a bisphosphonate, denosumab, a corticosteroid, or a combination thereof.


SEQUENCE LISTING (65 sequences)

SEQ ID NO: 1 - DNA, 1842 bp, Homo sapiens
  source 1..1842: mol_type = other DNA
ctgctggggagtcgcctacgcctacttcctgccgggaggaggggctcgagttccgcgtcgtcgcgcagagctgactctgggaggcgtttgggcccagagaagtggatccgccgcttgcgccgcatggagtccgaatcggaaagcggggctgctgctgacacccccccactggagaccctaagcttccatggtgatgaagagattatcgaggtggtagaacttgatcccggtccgccggacccagatgacctggcccaggagatggaagatgtggactttgaggaagaagaggaggaagagggcaacgaagagggctgggttctagaaccccaggaaggggtggtcggcagcatggagggccccgacgatagcgaggtcacctttgcattgcactcagcatctgtgttttgtgtgagcctggaccccaagaccaataccttggcagtgaccgggggtgaagatgacaaagccttcgtatggcggctcagcgatggggagctgctctttgagtgtgcaggccataaagactctgtgacttgtgctggtttcagccatgactccactctagtggccacaggggacatgagtggcctcttgaaagtgtggcaggtggacactaaggaggaggtctggtcctttgaagcgggagacctggagtggatggagtggcatcctcgggcacctgtcctgttggcgggcacagctgacggcaacacctggatgtggaaagtcccgaatggtgactgcaagaccttccagggtcccaactgcccagccacctgtggccgagtcctccctgatgggaagagagctgtggtaggctatgaagatgggaccatcaggatttgggacctgaagcagggaagccctatccatgtactgaaagggactgagggtcaccagggcccactcacctgtgttgctgccaaccaggatggcagcttgatcctaactggctctgtggactgccaggccaagctggtcagtgccaccaccggcaaggtggtgggtgtttttagacctgagactgtggcctcccagcccagcctgggagaaggggaggagagtgagtccaactcggtggagtccttgggcttctgcagtgtgatgcccctggcagctgttggctacctggatgggaccttggccatctatgacctggctacgcagactcttaggcatcagtgtcagcaccagtcgggcatcgtgcagctgctgtgggaggcaggcactgccgtggtatatacctgcagcctggatggcatcgtgcgcctctgggacgcccggaccggccgcctgcttactgactaccggggccacacggctgagatcctggactttgccctcagcaaagatgcctccctggtggtgaccacgtcaggagaccacaaagcgaaagtattttgtgtccaaaggcctgaccgttaatggctgcagcccctgcctgtgtgtctggtgttgaggggacgaagggacccctgcccctgtctgccagcagaggcagtagggcacagagggaagaggagggtggggccctggatgactttccagcctcttcaactgacttgctcccctctccttttcttctctttagagacccagcccagggccctcccacccttgtccagacctggtgggcccttcagagggaggggtggacctgtttctctttcactttcatttgctggtgtgagccatggggtgtgtatttgtatgtggggagtaggtgtttgaggttcccgttctttcccttcccaagtctctgggggtggaaaggaggaagagatactagttaaagattttaaaaatgtaaataaaatatacttcccagaaaaaaaaaaa

SEQ ID NO: 2 - DNA, 4128 bp, Homo sapiens
  source 1..4128: mol_type = other DNA
aaagatagatcctgctccaggagccgggaagccttgccctggccagctgtgctgggcacctcccctgcctgcttcctggcccacttgcaggcaaggtgagggcatgcgaatggctgccactgcctgggcggggctccaagggccacccctccccaccctctgtcccgcagtgaggacgggactctactgccgagaccaggctcacgctgagaggtgggccatgacctccgagacctcttccggaagccactgtgccaggagcaggatgctgcggcgacgggcccaggaagaggacagcaccgtcctgatcgatgtgagcccccctgaggcagagaagaggggctcttacgggagcacagcccacgcctcggagccaggtggacagcaagcggccgcctgcagagctgggagtcctgccaagccccggatcgcagacttcgtcctcgtttgggaggaggacctgaagctagacaggcagcaggacagtgccgcccgggacagaacagacatgcacaggacctggcgggagacttttctggataatcttcgtgcggctgggctgtgtgtagaccagcaggacgtccaggacgggaacaccacagtgcactacgccctcctcagcgcctcctgggctgtgctctgctactacgccgaagacctgcgcctgaagctgcccttgcaggagttacccaaccaggcctccaactggtcggccggcctgctggcatggctgggcatccccaacgtcctgctggaggttgtgccagacgtaccccccgagtactactcctgccggttcagagtgaacaagctgccacgcttcctcgggagtgacaaccaggacaccttcttcacaagcaccaagaggcaccaaattctgtttgagatcctggccaagaccccgtatggccacgagaagaaaaacctgcttgggatccaccagctgctggcagagggtgtcctcagtgccgccttccccctgcatgacggccccttcaagacgcccccagagggcccgcaggctccacgcctcaaccagcgccaagtccttttccagcactgggcgcgctggggcaagtggaacaagtaccagcccctggaccacgtgcgcaggtacttcggggagaaggtggccctctacttcgcctggctcgggttttacacaggctggctcctgccagcggcagtggtgggcacactggtgttcctggtgggctgcttcctggtgttctcagacatacccacgcaggaactgtgtggcagcaaggacagcttcgagatgtgcccactttgcctcgactgccctttctggctgctctccagcgcctgtgccctggcccaggccggccggctgttcgaccacggcggcaccgtgttcttcagcttgttcatggcactgtgggccgtgctgctgctggagtactggaagcggaagagcgccacgctggcctaccgctgggactgctctgactacgaggacactgaggagaggcctcggccccagtttgccgcctcagcccccatgacagccccgaaccccatcacgggtgaggacgagccctacttccctgagaggagccgcgcgcgccgcatgctggccggctctgtggtgatcgtggtgatggtggccgtggtggtcatgtgcctcgtgtctatcatcctgtaccgtgccatcatggccatcgtggtgtccaggtcgggcaacacccttctcgcagcctgggcctctcgcatcgccagcctcacggggtctgtagtgaacctcgtcttcatcctcatcctctccaagatctatgtatccctggcccacgtcctgacacgatgggaaatgcaccgcacccagaccaagttcgaggacgccttcaccctcaaggtgttcatcttccagttcgtcaacttctactcctcacccgtctacattgccttcttcaagggcaggtttgtgggatacccaggcaactaccacaccttgtttggagtccgcaatgaggagtgcgcggctggaggctgcctgatcgagctggcacaggagctcctggtcatcatggtgggcaagcaggtcatcaacaacatgcaggaggtcctcatcccgaagctaaagggctggtggcagaagttccggcttcgctccaagaagaggaaggcgggagcttctgcaggggctagccaggggccctgggaggacgactatgagcttgtgccctgtgagggtctgtttgacgagtacctggaaatggtgctgcagttcggcttcgtcaccatcttcgtggccgcctgtccgctcgcgccgctcttcgccctgctcaacaactgggtggagatccgcttggacgcgcgcaagttcgtctgcgagtaccggcgcccggtggccgagcgcgcccaggacatcggcatctggttccacatcctggcgggcctcacgcacctggcggtcatcagcaacgccttcctcctggccttctcgtccgacttcctgccgcgcgcctactaccggtggacccgcgcccacgacctgcgcggcttcctcaacttcacgctggcgcgagccccgtcctccttcgccgccgcgcacaaccgcacgtgcaggtatcgggctttccgggatgacgatggacattattcccagacctactggaatcttcttgccatccgcctggccttcgtcattgtgtttgagcatgtggttttctccgttggccgcctcctggacctcctggtgcctgacatcccagagtctgtggagatcaaagtgaagcgggagtactacctggctaagcaggcactggctgagaatgaggttctttttggaacgaacggaacaaaggatgagcagcccgagggctcagagctcagctcccactggacacccttcacggttcccaaggccagccagctgcagcagtgacgcctggaaggacatctggtggtccttaggggagtggcccctcctgagccctgcgagcagcgtccttttcctcttccctcaggcagcggctgtgtgaaccgctggctgctgttgtgcctcatctctgggcacattgcctgcttccccccagcgccggcttctctcctcagagcgcctgtcactccatccccggcagggagggaccgtcagctcacaaggccctctttgtttcctgctcccagacataagcccaaggggcccctgcacccaagggaccctgtccctcggtggcctccccaggcccctggacacgacagttctcctcaggcaggtgggctttgtggtcctcgccgcccctggccacatcgccctctcctcttacacctggtgaccttcgaatgtttcagagcgcagggccgttctccctcgtgtcctctggacccacccgccccttcctgccctgtttgcgcagggacatcacccacatgccccagctctcggaccctgcagctctgtgtcccaggccacagcaaaggtctgttgaacccctccctccattcccagttatctgggtcctctggattcttctgtttcttgaatcaggctctgctttccccctagccactacaggcagcctctgacagtgccgctttacttgcattctgcagcaattacatgtgtccttttgatccttgcccaacttccctccctctcccagctcctggcccctggcccagggcccctcttgctgtttttacctctgttccttggggcctagtacccagcaagcacccaaatgggggaggttttgggatgagaggaggaaacgtgtatacctgtaacatctggtggctcttcccccagaagtttgtgttcatacataattgttttccacgctggatcataatgtgacgtgcagttctgccctgtgctggggagccacatgaagcttcccctggctaacttgctaccccgcagcaatcccagtgtggccgtctgcttgctaaaaaatggatctgtgctcatctgtattgatgtccttggagttctacaagtggaacttaagtgtcaaaaagaatatgtggtttttagctgagcgtggtggctcacacctgtaatcccaggactttgggaggctgaggcaggaggattacaaggtcaggagttcgggactagcctgtccaacatggtgaaaccctgtctttactaaaaatgcaaaaatt

SEQ ID NO: 3 - DNA, 10661 bp, Homo sapiens
  source 1..10661: mol_type = other DNA
cgagatcccggggagccagcttgctgggagagcgggacggtccggagcaagcccagaggcagaggaggcgacagagggaaaaagggccgagctagccgctccagtgctgtacaggagccgaagggacgcaccacgccagccccagcccggctccagcgacagccaacgcctcttgcagcgcggcggcttcgaagccgccgcccggagctgccctttcctcttcggtgaagtttttaaaagctgctaaagactcggaggaagcaaggaaagtgcctggtaggactgacggctgcctttgtcctcctcctctccaccccgcctccccccaccctgccttccccccctcccccgtcttctctcccgcagctgcctcagtcggctactctcagccaacccccctcaccacccttctccccacccgcccccccgcccccgtcggcccagcgctgccagcccgagtttgcagagaggtaactccctttggctgcgagcgggcgagctagctgcacattgcaaagaaggctcttaggagccaggcgactggggagcggcttcagcactgcagccacgacccgcctggttaggctgcacgcggagagaaccctctgttttcccccactctctctccacctcctcctgccttccccaccccgagtgcggagccagagatcaaaagatgaaaaggcagtcaggtcttcagtagccaaaaaacaaaacaaacaaaaacaaaaaagccgaaataaaagaaaaagataataactcagttcttatttgcacctacttcagtggacactgaatttggaaggtggaggattttgtttttttcttttaagatctgggcatcttttgaatctacccttcaagtattaagagacagactgtgagcctagcagggcagatcttgtccaccgtgtgtcttcttctgcacgagactttgaggctgtcagagcgctttttgcgtggttgctcccgcaagtttccttctctggagcttcccgcaggtgggcagctagctgcagcgactaccgcatcatcacagcctgttgaactcttctgagcaagagaaggggaggcggggtaagggaagtaggtggaagattcagccaagctcaaggatggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtgaagcattggaaaccctatttccccaccccagctcatgccccctttcagatgtcttctgcctgttataactctgcactactcctctgcagtgccttggggaatttcctctattgatgtacagtctgtcatgaacatgttcctgaattctatttgctgggctttttttttctctttctctcctttctttttcttcttccctccctatctaaccctcccatggcaccttcagactttgcttcccattgtggctcctatctgtgttttgaatggtgttgtatgcctttaaatctgtgatgatcctcatatggcccagtgtcaagttgtgcttgtttacagcactactctgtgccagccacacaaacgtttacttatcttatgccacgggaagtttagagagctaagattatctggggaaatcaaaacaaaaacaagcaaacaaaaaaaaaaagcaaaaacaaaacaaaaaataagccaaaaaaccttgctagtgttttttcctcaaaaataaataaataaataaataaatacgtacatacatacacacatacatacaaacatatagaaatccccaaagaggccaatagtgacgagaaggtgaaaattgcaggcccatggggagttactgattttttcatctcctccctccacgggagactttattttctgccaatggctattgccattagagggcagagtgaccccagagctgagttgggcaggggggtggacagagaggagaggacaaggagggcaatggagcatcagtacctgcccacagccttggtccctgggggctagactgctcaactgtggagcaattcattatactgaaaatgtgcttgttgttgaaaatttgtctgcatgttaatgcctcacccccaaacccttttctctctcactctctgcctccaacttcagattgactttcaatagtttttctaagacctttgaactgaatgttctcttcagccaaaacttggcgacttccacagaaaagtctgaccactgagaagaaggagagcagagatttaaccctttgtaaggccccatttggatccaggtctgctttctcatgtgtgagtcagggaggagctggagccagaggagaagaaaatgatagcttggctgttctcctgcttaggacactgactgaatagttaaactctcactgccactaccttttccccacctttaaaagacctgaatgaagttttctgccaaactccgtgaagccacaagcaccttatgtcctcccttcagtgttttgtgggcctgaatttcatcacactgcatttcagccatggtcatcaagcctgtttgcttcttttgggcatgttcacagattctctgttaagagcccccaccaccaagaaggttagcaggccaacagctctgacatctatctgtagatgccagtagtcacaaagatttcttaccaactctcagatcgctggagcccttagacaaactggaaagaaggcatcaaagggatcaggcaagctgggcgtcttgcccttgtcccccagagatgataccctcccagcaagtggagaagttctcacttccttctttagagcagctaaaggggctacccagatcagggttgaagagaaaactcaattaccagggtgggaagaatgaaggcactagaaccagaaaccctgcaaatgctcttcttgtcacccagcatatccacctgcagaagtcatgagaagagagaaggaacaaagaggagactctgactactgaattaaaatcttcagcggcaaagcctaaagccagatggacaccatctggtgagtttactcatcatcctcctctgctgctgattctgggctctgacattgcccatactcactcagattccccacctttgttgctgcctcttagtcagagggaggccaaaccattgagactttctacagaaccatggcttctttcggaaaggtctggttggtgtggctccaatactttgccacccatgaactcagggtgtgccctgggacactggttttatatagtcttttggcacacctgtgttctgttgacttcgttcttcaagcccaagtgcaagggaaaatgtccacctactttctcatcttggcctctgcctccttacttagctcttaatctcatctgttgaactcaagaaatcaagggccagtcatcaagctgcccattttaattgattcactctgtttgttgagaggatagtttctgagtgacatgatatgatccacaagggtttccttccctgatttctgcattgatattaatagccaaacgaacttcaaaacagctttaaataacaagggagaggggaacctaagatgagtaatatgccaatccaagactgctggagaaaactaaagctgacaggttccctttttggggtgggatagacatgttctggttttctttattattacacaatctggctcatgtacaggatcacttttagctgttttaaacagaaaaaaatatccaccactcttttcagttacactaggttacattttaataggtcctttacatctgttttggaatgattttcatcttttgtgatacacagattgaattatatcattttcatatctctccttgtaaatactagaagctctcctttacatttctctatcaaatttttcatctttatgggtttcccaattgtgactcttgtcttcatgaatatatgtttttcatttgcaaaagccaaaaatcagtgaaacagcagtgtaattaaaagcaacaactggattactccaaatttccaaatgacaaaactagggaaaaatagcctacacaagcctttaggcctactctttctgtgcttgggtttgagtgaacaaaggagattttagcttggctctgttctcccatggatgaaaggaggaggattttttttttcttttggccattgatgttctagccaatgtaattgacagaagtctcattttgcatgcgctctgctctacaaacagagttggtatggttggtatactgtactcacctgtgagggactggccactcagacccacttagctggtgagctagaagatgaggatcactcactggaaaagtcacaaggaccatctccaaacaagttggcagtgctcgatgtggacgaagagtgaggaagagaaaaagaaggagcaccagggagaaggctccgtctgtgctgggcagcagacagctgccaggatcacgaactctgtagtcaaagaaaagagtcgtgtggcagtttcagctctcgttcattgggcagctcgcctaggcccagcctctgagctgacatgggagttgttggattctttgtttcatagctttttctatgccataggcaatattgttgttcttggaaagtttattatttttttaactcccttactctgagaaagggatattttgaaggactgtcatatatctttgaaaaaagaaaatctgtaatacatatatttttatgtatgttcactggcactaaaaaatatagagagcttcattctgtcctttgggtagttgctgaggtaattgtccaggttgaaaaataatgtgctgatgctagagtccctctctgtccatactctacttctaaatacatataggcatacatagcaagttttatttgacttgtactttaagagaaaatatgtccaccatccacatgatgcacaaatgagctaacattgagcttcaagtagcttctaagtgtttgtttcattaggcacagcacagatgtggcctttccccccttctctcccttgatatctggcagggcataaaggcccaggccacttcctctgccccttcccagccctgcaccaaagctgcatttcaggagactctctccagacagcccagtaactacccgagcatggcccctgcatagccctggaaaaataagaggctgactgtctacgaattatcttgtgccagttgcccaggtgagagggcactgggccaagggagtggttttcatgtttgacccactacaaggggtcatgggaatcaggaatgccaaagcaccagatcaaatccaaaacttaaagtcaaaataagccattcagcatgttcagtttcttggaaaaggaagtttctacccctgatgcctttgtaggcagatctgttctcaccattaatctttttgaaaatcttttaaagcagtttttaaaaagagagatgaaagcatcacattatataaccaaagattacattgtacctgctaagataccaaaattcataagggcagggggggagcaagcattagtgcctctttgataagctgtccaaagacagactaaaggactctgctggtgactgacttataagagctttgtgggtttttttttccctaataatatacatgtttagaagaattgaaaataatttcgggaaaatgggattatgggtccttcactaagtgattttataagcagaactggctttccttttctctagtagttgctgagcaaattgttgaagctccatcattgcatggttggaaatggagctgttcttagccactgtgtttgctagtgcccatgttagcttatctgaagatgtgaaacccttgctgataagggagcatttaaagtactagattttgcactagagggacagcaggcagaaatccttatttctgcccactttggatggcacaaaaagttatctgcagttgaaggcagaaagttgaaatacattgtaaatgaatatttgtatccatgtttcaaaattgaaatatatatatatatatatatatatatatatatatatatatagtgtgtgtgtgtgttctgatagctttaactttctctgcatctttatatttggttccagatcacacctgatgccatgtacttgtgagagaggatgcagttttgttttggaagctctctcagaacaaacaagacacctggattgatcagttaactaaaagttttctcccctattgggtttgacccacaggtcctgtgaaggagcagagggataaaaagagtagaggacatgatacattgtactttactagttcaagacagatgaatgtggaaagcataaaaactcaatggaactgactgagatttaccacagggaaggcccaaacttggggccaaaagcctacccaagtgattgaccagtggccccctaatgggacctgagctgttggaagaagagaactgttccttggtcttcaccatccttgtgagagaagggcagtttcctgcattggaacctggagcaagcgctctatctttcacacaaattccctcacctgagattgaggtgctcttgttactgggtgtctgtgtgctgtaattctggttttggatatgttctgtaaagattttgacaaatgaaaatgtgtttttctctgttaaaacttgtcagagtactagaagttgtatctctgtaggtgcaggtccatttctgcccacaggtagggtgtttttctttgattaagagattgacacttctgttgcctaggacctcccaactcaaccatttctaggtgaaggcagaaaaatccacattagttactcctcttcagacatttcagctgagataacaaatcttttggaattttttcacccatagaaagagtggtagatatttgaatttagcaggtggagtttcatagtaaaaacagcttttgactcagctttgatttatcctcatttgatttggccagaaagtaggtaatatgcattgattggcttctgattccaattcagtatagcaaggtgctaggttttttcctttccccacctgtctcttagcctggggaattaaatgagaagccttagaatgggtggcccttgtgacctgaaacacttcccacataagctacttaacaagattgtcatggagctgcagattccattgcccaccaaagactagaacacacacatatccatacaccaaaggaaagacaattctgaaatgctgtttctctggtggttccctctctggctgctgcctcacagtatgggaacctgtactctgcagaggtgacaggccagatttgcattatctcacaaccttagcccttggtgctaactgtcctacagtgaagtgcctggggggttgtcctatcccataagccacttggatgctgacagcagccaccatcagaatgacccacgcaaaaaaaagaaaaaaaaaattaaaaagtcccctcacaacccagtgacacctttctgctttcctctagactggaacattgattagggagtgcctcagacatgacattcttgtgctgtccttggaattaatctggcagcaggagggagcagactatgtaaacagagataaaaattaattttcaatattgaaggaaaaaagaaataagaagagagagagaaagaaagcatcacacaaagattttcttaaaagaaacaattttgcttgaaatctctttagatggggctcatttctcacggtggcacttggcctccactgggcagcaggaccagctccaagcgctagtgttctgttctctttttgtaatcttggaatcttttgttgctctaaatacaattaaaaatggcagaaacttgtttgttggactacatgtgtgactttgggtctgtctctgcctctgctttcagaaatgtcatccattgtgtaaaatattggcttactggtctgccagctaaaacttggccacatcccctgttatggctgcaggatcgagttattgttaacaaagagacccaagaaaagctgctaatgtcctcttatcattgttgttaatttgttaaaacataaagaaatctaaaatttcaaaaaa

SEQ ID NO: 4 - DNA, 3641 bp, Homo sapiens
  source 1..3641: mol_type = other DNA
gacactgaatttggaaggtggaggattttgtttttttcttttaagatctgggcatcttttgaatctacccttcaagtattaagagacagactgtgagcctagcagggcagatcttgtccaccgtgtgtcttcttctgcacgagactttgaggctgtcagagcgctttttgcgtggttgctcccgcaagtttccttctctggagcttcccgcaggtgggcagctagctgcagcgactaccgcatcatcacagcctgttgaactcttctgagcaagagaaggggaggcggggtaagggaagtaggtggaagattcagccaagctcaaggatggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgcagcagcagcagcagcagcagcagcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagaaaaattccgggttggcaattgcaagcatctcaaaatgaccagaccctgaagaaaggctgacttgcctcattcaaaatgagggctctagagggctctagtggatagtctggagaaacctggcgtctgaggcttaggagcttaggtttttgctcctcaacacagactttgacgttggggttgggggctactctcttgattgctgactccctccagcgggaccaatagtgttttcctacctcacagggatgttgtgaggacgggctgtagaagtaatagtggttaccactcatgtagttgtgagtatcatgattattgtttcctgtaatgtggcttggcattggcaaagtgctttttgattgttcttgatcacatatgatgggggccaggcactgactcaggcggatgcagtgaagctctggctcagtcgcttgcttttcgtggtgtgctgccaggaagaaactttgctgatgggactcaaggtgtcaccttggacaagaagcaactgtgtctgtctgaggttcctgtggccatctttatttgtgtattaggcaattcgtatttcccccttaggttctagccttctggatcccagccagtgacctagatcttagcctcaggccctgtcactgagctgaaggtagtagctgatccacagaagttcagtaaacaaggaccagatttctgcttctccaggagaagaagccagccaacccctctcttcaaacacactgagagactacagtccgactttccctcttacatctagccttactgtagccacactccttgattgctctctcacatcacatgcttctcttcatcagttgtaagcctctcattcttctcccaagccagactcaaatattgtattgatgtcaaagaagaatcacttagagtttggaatatcttgttctctctctgctccatagcttccatattgacaccagtttctttctagtggagaagtggagtctgtgaagccagggaaacacacatgtgagagtcagaaggactctccctgacttgcctggggcctgtctttcccaccttctccagtctgtctaaacacacacacacacacacacacacacacacacacacacacacacgctctctctctctctccccccccaacacacacacactctctctctcacacacacacacatacacacacacttctttctctttcccctgactcagcaacattctggagaaaagccaaggaaggacttcaggaggggagtttcccccttctcagggcagaattttaatctccagaccaacaagaagttccctaatgtggattgaaaggctaatgaggtttatttttaactactttctatttgtttgaatgttgcatatttctactagtgaaattttcccttaataaagccattaatacacccaaaaaaaaaaaaaaaa

SEQ ID NO: 5 - DNA, 1575 bp, Homo sapiens
  source 1..1575: mol_type = other DNA
ctacgcccaggactggcctaacctccgctgcctaaccgtcatccccctgcagccacccttcccagagtcctttgcccaggccaccccaggcttcttggcagccctgccgggccacttgtcttcatgtctgccagggggaggtgggaaggaggtgggaggagggcgtgcagaggcagtctgggcttggccagagctcagggtgctgagcgtgtgaccagcagtgagcagaggccggccatggccagcctggggctgctgctcctgctcttactgacagcactgccaccgctgtggtcctcctcactgcctgggctggacactgctgaaagtaaagccaccattgcagacctgatcctgtctgcgctggagagagccaccgtcttcctagaacagaggctgcctgaaatcaacctggatggcatggtgggggtccgagtgctggaagagcagctaaaaagtgtccgggagaagtgggcccaggagcccctgctgcagccgctgagcctgcgcgtggggatgctgggggagaagctggaggctgccatccagagatccctccactacctcaagctgagtgatcccaagtacctaagagagttccagctgaccctccagcccgggttttggaagctcccacatgcctggatccacactgatgcctccttggtgtaccccacgttcgggccccaggactcattctcagaggagagaagtgacgtgtgcctggtgcagctgctgggaaccgggacggacagcagcgagccctgcggcctctcagacctctgcaggagcctcatgaccaagcccggctgctcaggctactgcctgtcccaccaactgctcttcttcctctgggccagaatgagggggtgcacacagggaccactccaacagagccaggactatatcaacctcttctgcgccaacatgatggacttgaaccgcagagctgaggccatcggatacgcctaccctacccgggacatcttcatggaaaacatcatgttctgtggaatgggcggcttctccgacttctacaagctccggtggctggaggccattctcagctggcagaaacagcaggaaggatgcttcggggagcctgatgctgaagatgaagaattatctaaagctattcaatatcagcagcatttttcgaggagagtgaagaggcgagaaaaacaatttccagatggctgctcctcccacaacacagccacagcagtggcagccctgggtggcttcctatacatcctggcagaataccccccagcaaacagagagccacacccatccacaccgccaccaccaagcagccgctgagacggacggttccatgccagctgcctggaggaggaacagacccctttagtcctcatcccttagatcctggagggcacggatcacatcctgggaagaaggcatctggaggataagcaaagccaccccgacacccaatcttggaagccctgagtaggcagggccagggtaggtgggggccgggagggacccaggtgtgaacggatgaataaagttcaactgcaactgaaaaaaaaaaaaaaaaaaaaaaaaaaa

SEQ ID NO: 6 - DNA, 2175 bp, Homo sapiens
  source 1..2175: mol_type = other DNA
tttcccgccctctagtcgtgcgcggatctgacgcctgacgtaattgcgtagacgccattttagccggtcagacaagcactggacgtggcggccattttgttttggacaccgagcaggagctggcggccgctgcagacgaaaggcaggaaagggcaggccgggtgagcagacggatcggccgactagacagccaaccagcaacaacgaactgagctcgcatactaccgcttacgcatctaaccaaccgcccatctagctaacccgagcccctccaccgtcaactcaggttcggccggtccccggcccgcctgccggagccgtggtggcagccccgggaggagcactggcgtctgtttccttcgattctcgggattcgaagatggctgcacagtcagcgccgaaagttgtgctaaaaagcaccaccaagatgtctctaaatgagcgctttactaatatgctgaagaacaaacagccgacgccagtgaatattcgggcttcgatgcagcaacaacagcagctagccagtgccagaaacagaagactggcccagcagatggagaatagaccctctgtccaggcagcattaaaacttaagcagaagagcttaaagcagcgcctgggtaagagtaacatccaggcacggttaggccgacccataggggccctggccaggggagcaatcggaggacgaggcctacccataatccagagaggcttgcccagaggaggactacgtgggggacgtgccaccagaaccctacttaggggcgggatgtcactccgaggtcaaaacctgctccgaggtggacgagccgtagctccccgaatgggcttaagaagaggtggtgttcgaggtcgtggaggtcctgggagagggggcctagggcgtggagctatgggtcgtggcggaatcggtggtagaggtcggggtatgataggtcggggaagagggggctttggaggccgaggccgaggccgtggacgagggagaggtgcccttgctcgccctgtattgaccaaggagcagctggacaaccaattggatgcatatatgtcgaaaacaaaaggacacctggatgctgagttggatgcctacatggcgcagacagatcccgaaaccaatgattgaagcctgcccatcctcccatgagagactcttgttagtcaacacatctgtaaataaccttgagataacagatgagaagaaatctgattgatgctggatggacctatcacaataggctgtggacttacttgccaccagcttgtgcatttagtgtgttccttttactttttgatactgtgttgtatgaaacccttttgtcctttgatttggttttttgtttttgtttttttaggggggagggggggtttcccctcctttgcccagacttctctttgaacacaaatgcattagccttgtggctagaacaccctcttcctacctctgtctcccctcacttgtcatatgctctgacatgctaacatttcttttgttcatccctgttgcccccacagaaacatcccagaaaaaccggtcagtgttccttcctccctgatccttaggtttctgaaatagggttctgttacatcctcttcgatagcctgtttaaaatgtttagaaggtctggagctcaaaaatgcgttcttccacattgataatttagtaaactgagaacattgacatcactacagggcagcataagaggttgcttacatgtggtagcagctctggtttgattcaagttgctaccatgtacattgacagcacatataccataaccagcgtgttgggttgaattgcactttctacctttgtatgagatttacagactttccttctgggtttgtatcatgaccagaggggtactatagggttggtttatactgcaatatagaggatcagaagccatttgatttggtaggtgtgtcagaagggagaatgatggcagacgaactgctggaagaggtcagaagatagccatgctaaaatgcaattatatcctcatgtttatcccaaactaatcttggacttttccactcattagctttgttttgcccttgtttcccttgaaggtttaagttcaaccatattctgtcaactgttcagtttcagtggaatcttgtatttctggttcattataacaaactgttcgcttaaatccaaaaaaaaaa

SEQ ID NO: 7 - DNA, 5927 bp, Homo sapiens
  source 1..5927: mol_type = other DNA
tcgtcggagcagacgggagtttctcctcggggtcggagcaggaggcacgcggagtgtgaggccacgcatgagcggacgctaaccccctccccagccacaaagagtctacatgtctagggtctagacatgttcagctttgtggacctccggctcctgctcctcttagcggccaccgccctcctgacgcacggccaagaggaaggccaagtcgagggccaagacgaagacatcccaccaatcacctgcgtacagaacggcctcaggtaccatgaccgagacgtgtggaaacccgagccctgccggatctgcgtctgcgacaacggcaaggtgttgtgcgatgacgtgatctgtgacgagaccaagaactgccccggcgccgaagtccccgagggcgagtgctgtcccgtctgccccgacggctcagagtcacccaccgaccaagaaaccaccggcgtcgagggacccaagggagacactggcccccgaggcccaaggggacccgcaggcccccctggccgagatggcatccctggacagcctggacttcccggaccccccggaccccccggacctcccggaccccctggcctcggaggaaactttgctccccagctgtcttatggctatgatgagaaatcaaccggaggaatttccgtgcctggccccatgggtccctctggtcctcgtggtctccctggcccccctggtgcacctggtccccaaggcttccaaggtccccctggtgagcctggcgagcctggagcttcaggtcccatgggtccccgaggtcccccaggtccccctggaaagaatggagatgatggggaagctggaaaacctggtcgtcctggtgagcgtgggcctcctgggcctcagggtgctcgaggattgcccggaacagctggcctccctggaatgaagggacacagaggtttcagtggtttggatggtgccaagggagatgctggtcctgctggtcctaagggtgagcctggcagccctggtgaaaatggagctcctggtcagatgggcccccgtggcctgcctggtgagagaggtcgccctggagcccctggccctgctggtgctcgtggaaatgatggtgctactggtgctgccgggccccctggtcccaccggccccgctggtcctcctggcttccctggtgctgttggtgctaagggtgaagctggtccccaagggccccgaggctctgaaggtccccagggtgtgcgtggtgagcctggcccccctggccctgctggtgctgctggccctgctggaaaccctggtgctgatggacagcctggtgctaaaggtgccaatggtgctcctggtattgctggtgctcctggcttccctggtgcccgaggcccctctggaccccagggccccggcggccctcctggtcccaagggtaacagcggtgaacctggtgctcctggcagcaaaggagacactggtgctaagggagagcctggccctgttggtgttcaaggaccccctggccctgctggagaggaaggaaagcgaggagctcgaggtgaacccggacccactggcctgcccggaccccctggcgagcgtggtggacctggtagccgtggtttccctggcgcagatggtgttgctggtcccaagggtcccgctggtgaacgtggttctcctggccctgctggccccaaaggatctcctggtgaagctggtcgtcccggtgaagctggtctgcctggtgccaagggtctgactggaagccctggcagccctggtcctgatggcaaaactggcccccctggtcccgccggtcaagatggtcgccccggacccccaggcccacctggtgcccgtggtcaggctggtgtgatgggattccctggacctaaaggtgctgctggagagcccggcaaggctggagagcgaggtgttcccggaccccctggcgctgtcggtcctgctggcaaagatggagaggctggagctcagggaccccctggccctgctggtcccgctggcgagagaggtgaacaaggccctgctggctcccccggattccagggtctccctggtcctgctggtcctccaggtgaagcaggcaaacctggtgaacagggtgttcctggagaccttggcgcccctggcccctctggagcaagaggcgagagaggtttccctggcgagcgtggtgtgcaaggtccccctggtcctgctggtccccgaggggccaacggtgctcccggcaacgatggtgctaagggtgatgctggtgcccctggagctcccggtagccagggcgcccctggccttcagggaatgcctggtgaacgtggtgcagctggtcttccagggcctaagggtgacagaggtgatgctggtcccaaaggtgctgatggctctcctggcaaagatggcgtccgtggtctgactggccccattggtcctcctggccctgctggtgcccctggtgacaagggtgaaagtggtcccagcggccctgctggtcccactggagctcgtggtgcccccggagaccgtggtgagcctggtccccccggccctgctggctttgctggcccccctggtgctgacggccaacctggtgctaaaggcgaacctggtgatgctggtgctaaaggcgatgctggtccccctggccctgccggacccgctggaccccctggccccattggtaatgttggtgctcctggagccaaaggtgctcgcggcagcgctggtccccctggtgctactggtttccctggtgctgctggccgagtcggtcctcctggcccctctggaaatgctggaccccctggccctcctggtcctgctggcaaagaaggcggcaaaggtccccgtggtgagactggccctgctggacgtcctggtgaagttggtccccctggtccccctggccctgctggcgagaaaggatcccctggtgctgatggtcctgctggtgctcctggtactcccgggcctcaaggtattgctggacagcgtggtgtggtcggcctgcctggtcagagaggagagagaggcttccctggtcttcctggcccctctggtgaacctggcaaacaaggtccctctggagcaagtggtgaacgtggtccccctggtcccatgggcccccctggattggctggaccccctggtgaatctggacgtgagggggctcctggtgccgaaggttcccctggacgagacggttctcctggcgccaagggtgaccgtggtgagaccggccccgctggaccccctggtgctcctggtgctcctggtgcccctggccccgttggccctgctggcaagagtggtgatcgtggtgagactggtcctgctggtcccgccggtcctgtcggccctgttggcgcccgtggccccgccggaccccaaggcccccgtggtgacaagggtgagacaggcgaacagggcgacagaggcataaagggtcaccgtggcttctctggcctccagggtccccctggccctcctggctctcctggtgaacaaggtccctctggagcctctggtcctgctggtccccgaggtccccctggctctgctggtgctcctggcaaagatggactcaacggtctccctggccccattgggccccctggtcctcgcggtcgcactggtgatgctggtcctgttggtccccccggccctcctggacctcctggtccccctggtcctcccagcgctggtttcgacttcagcttcctgccccagccacctcaagagaaggctcacgatggtggccgctactaccgggctgatgatgccaatgtggttcgtgaccgtgacctcgaggtggacaccaccctcaagagcctgagccagcagatcgagaacatccggagcccagagggcagccgcaagaaccccgcccgcacctgccgtgacctcaagatgtgccactctgactggaagagtggagagtactggattgaccccaaccaaggctgcaacctggatgccatcaaagtcttctgcaacatggagactggtgagacctgcgtgtaccccactcagcccagtgtggcccagaagaactggtacatcagcaagaaccccaaggacaagaggcatgtctggttcggcgagagcatgaccgatggattccagttcgagtatggcggccagggctccgaccctgccgatgtggccatccagctgaccttcctgcgcctgatgtccaccgaggcctcccagaacatcacctaccactgcaagaacagcgtggcctacatggaccagcagactggcaacctcaagaaggccctgctcctccagggctccaacgagatcgagatccgcgccgagggcaacagccgcttcacctacagcgtcactgtcgatggctgcacgagtcacaccggagcctggggcaagacagtgattgaatacaaaaccaccaagacctcccgcctgcccatcatcgatgtggcccccttggacgttggtgccccagaccaggaattcggcttcgacgttggccctgtctgcttcctgtaaactccctccatcccaacctggctccctcccacccaaccaactttccccccaacccggaaacagacaagcaacccaaactgaaccccctcaaaagccaaaaaatgggagacaatttcacatggactttggaaaatatttttttcctttgcattcatctctcaaacttagtttttatctttgaccaaccgaacatgaccaaaaaccaaaagtgcattcaaccttaccaaaaaaaaaaaaaaaaaaagaataaataaataactttttaaaaaaggaagcttggtccacttgcttgaagacccatgcgggggtaagtccctttctgcccgttgggcttatgaaaccccaatgctgccctttctgctcctttctccacaccccccttggggcctcccctccactccttcccaaatctgtctccccagaagacacaggaaacaatgtattgtctgcccagcaatcaaaggcaatgctcaaacacccaagtggcccccaccctcagcccgctcctgcccgcccagcacccccaggccctgggggacctggggttctcagactgccaaagaagccttgccatctggcgctcccatggctcttgcaacatctccccttcgtttttgagggggtcatgccgggggagccaccagcccctcactgggttcggaggagagtcaggaagggccacgacaaagcagaaacatcggatttggggaacgcgtgtcaatcccttgtgccgcagggctgggcgggagagactgttctgttccttgtgtaactgtgttgctgaaagactacctcgttcttgtcttgatgtgtcaccggggcaactgcctgggggcggggatgggggcagggtggaagcggctccccattttataccaaaggtgctacatctatgtgatgggtggggtggggagggaatcactggtgctatagaaattgagatgcccccccaggccagcaaatgttcctttttgttcaaagtctatttttattccttgatatttttctttttttttttttttttttgtggatggggacttgtgaatttttctaaaggtgctatttaacatgggaggagagcgtgtgcggctccagcccagcccgctgctcactttccaccctctctccacctgcctctggcttctcaggcctctgctctccgacctctctcctctgaaaccctcctccacagctgcagcccatcctcccggctccctcctagtctgtcctgcgtcctctgtccccgggtttcagagacaacttcccaaagcacaaagcagtttttccccctaggggtgggaggaagcaaaagactctgtacctattttgtatgtgtataataatttgagatgtttttaattattttgattgctggaataaagcatgtggaaatgacccaaacataa

SEQ ID NO: 8 - DNA, 4811 bp, Homo sapiens
  source 1..4811: mol_type = other DNA
tttcttctccggccctggcaggttccgggagcctcggctcgtgggtgccggaagtggaggcggttggtggggttggcggggctcagcgacgctgcgcgggtggcggtttgcgaactgcgggtggactgtgtagtgaccggcgtcccgctgtctcgccccgtggcgggtgagcgagggtgcgtggtgcgcggcggcggcggaacgaacgcggtgcgggcggggcgcccgccgcagggcccatggcctcctgcgcgagcatcgacatcgaggacgccacgcagcacctgcgggacatcctcaagctggaccggcccgcgggcggccccagtgcagagagcccacggccatccagtgcctacaatggggacctcaatggacttctggtcccagacccgctctgctcaggtgatagtacctcagcaaacaagactggtcttcggaccatgccacccattaacctgcaagagaagcaggtcatctgtctctcaggagatgatagctccacctgcattgggattttggccaaggaggtggagattgtggctagcagtgactctagcatttcaagcaaggcccggggaagcaacaaggtgaaaattcagcctgtcgccaagtatgactgggaacagaagtactactatggcaacctgattgctgtgtctaactccttcttggcctatgccattcgggctgccaacaatggctctgccatggtgcgggtgatcagcgtcagcacttcggagcggaccttgctcaagggcttcacaggcagtgtggctgatctggctttcgcgcacctcaactctccacagctggcctgcctggatgaggcaggcaacctgttcgtgtggcgcttggctctggttaatggcaaaattcaagaagagatcttggtccatattcggcagccagagggcacgccactgaaccactttcgcaggatcatctggtgccccttcatccctgaggagagcgaagactgctgtgaggagagcagcccaacagtggccctgctgcatgaagaccgggctgaggtgtgggacctggacatgctccgctccagccacagtacctggcctgtggatgttagccagatcaagcagggcttcattgtggtaaaaggtcatagcacgtgcctcagtgaaggagccctctctcctgatgggactgtgctggctactgcgagccacgatggctatgtcaagttctggcagatctacattgaggggcaagatgagccaaggtgtctgcacgagtggaaacctcatgatgggcggcccctctcctgcctcctgttctgtgacaaccataagaaacaagaccctgatgtccctttctggaggttccttattactggtgctgaccagaaccgagagttaaagatgtggtgtacagtatcctggacctgcctgcagactattcgcttctccccagatatcttcagctcagtgagtgtgccccctagcctcaaggtttgcttggacctctcagcagaatacctgattctcagcgatgtgcaacggaaggtcctctatgtgatggagctgctgcaaaaccaggaggagggccacgcctgcttcagctccatctcggagttcctgctcacccaccctgtgctgagctttggtatccaggttgtgagtcgctgccggctacggcacactgaggtgctgcctgccgaagaggaaaatgacagcctgggtgctgatggtacccatggagccggtgccatggagtctgcggccggtgtgctcatcaagctcttttgtgtgcatactaaggcactgcaagatgtgcagatccgcttccagccacagctgaaccctgatgtggtggccccactgcccacccacactgcccacgaggacttcacatttggagagtctcggcccgaactgggctctgagggcctggggtcagccgctcacggctcccagcctgacctccgacgaatcgtggagctgcctgcacctgccgacttcctcagtctgagcagtgagaccaagcccaagttgatgacacctgacgccttcatgacacctagcgcctccttgcagcagatcactgcctctcccagcagcagcagcagcggtagcagcagcagcagcagcagtagcagcagctcccttacagctgtgtctgccatgagcagcacctcagctgtggacccctccttgaccaggccacctgaggagctgaccttgagccccaagctgcagctggatggcagcctgacaatgagcagcagtggcagccttcaggcaagcccgcgtggcctcctgcctggcctgctcccagccccagctgacaaactgactcccaaggggccgggccaggtgcctactgccacctctgcactgtccctggagctgcaggaagtggagcccctggggctaccccaagcctcccctagccgcactcgttcccctgatgtcatctcctcagcttccactgccctgtcccaggacatccctgagattgcatctgaggccctgtcccgtggttttggctcctctgcaccagagggccttgagccagacagtatggcttcagccgcctcggcactgcacctgctgtccccacggccccggccagggcccgagctcggcccccagctcgggcttgatggaggccctggggatggagatcggcataataccccctccctcctggaggcagccttgacccaggaggcctcgactcctgacagtcaggtttggcccacagcacctgacattactcgtgagacctgcagcaccctggcagaaagccccaggaatggccttcaggaaaagcacaagagcctggccttccaccgaccaccatatcacctgctgcagcaacgtgacagccaggatgccagtgctgagcaaagtgaccatgatgatgaggtggccagccttgcctctgcttcaggaggctttggcaccaaagttcctgctccacggctgcctgccaaggactggaagaccaagggatcccctcgaacctcacccaagctcaagaggaaaagcaagaaggatgatggggatgcagccatgggatcccggctcacagagcaccaggtggcagagccccctgaggactggccagcactaatttggcaacagcagagagagctggcagagctgcggcacagccaggaagagctgctgcagcgtctgtgtacccaactcgaaggcctgcagagcacagtcacaggccacgtagaacgtgcccttgagactcggcacgagcaggaacagcggcggctggagcgagcactggctgaggggcagcagcggggagggcagctgcaggagcagctgacacaacagttgtcccaagcactgtcgtcagctgtagctgggcggctagagcgcagcatacgggatgagatcaagaagacagtccctccatgtgtctcaaggagtctggagcctatggcaggccaactgagcaactcagtggctaccaagctcacagctgtggagggcagcatgaaagagaacatctccaagctgctcaagtccaagaacttgactgatgccatcgcccgagcagctgcagacacattacaagggccgatgcaggctgcctaccgggaagccttccagagtgtggtgctgccggcctttgagaagagctgccaggccatgttccagcaaatcaatgatagcttccggctggggacacaggaatacttgcagcagctagaaagccacatgaagagccggaaggcacgggaacaggaggccagggagcctgtgctagcccagctgcggggcctggtcagcacactgcagagtgccactgagcagatggcagccaccgtggccggcagtgttcgtgctgaggtgcagcaccagctgcatgtggctgtgggcagcctgcaggagtccattttagcacaggtacagcgcatcgttaagggtgaggtgagtgtggcgctcaaggagcagcaggccgccgtcacctccagcatcatgcaggccatgcgctcagctgctggcacacctgtcccctctgcccaccttgactgccaggcccagcaagcccatatcctgcagctgctgcagcagggccacctcaatcaggccttccagcaggcgctgacagctgctgacctgaacctggtgctgtatgtgtgtgaaactgtggacccagcccaggtttttgggcagccaccctgcccgctctcccagcctgtgctcctttccctcatccagcagctggcatctgaccttggcactcgaactgacctcaagctcagctacctggaagaggccgtgatgcacctggaccacagtgaccccatcactcgggaccacatgggctccgttatggcccaggtgcgccaaaagctttttcagttcctgcaggctgagccacacaactcacttggcaaagcagctcggcgtctcagcctcatgctgcatggcctcgtgacccccagcctcccttagctgctaagcctgccttgcccaggggtgggatggcactgaaggccagcagacaggcctaggctggggcagggtcacggctggcctttacctgctcaggcccccatctctggggtgtttgggggtcagggagcagggagcactggccgtggtctacagcgtgtggtagtcagaaggtttagctgggcccagggcaggtattgcgcctgcttgggttctgccatgcctggagcatgaccctgagatcgtgacaccacttgagtggaattttccatgttcctttttacctctaatttggatctttttgtttttgaaaaacattgagaaattcaattaaatgcttttggaataaaatggagtatgtgtgtg

SEQ ID NO: 9 - DNA, 4654 bp, Homo sapiens
  source 1..4654: mol_type = other DNA
ggcggcggctggaggagagcgcggtggagagccgagcgggcgggcggcgggtgcggagcgggcgagggagcgcgcgcggccgccacaaagctcgggcgccgcggggctgcatgcggcgtacctggcccggcgcggcgactgctctccgggctggcgggggccggccgcgagccccgggggccccgaggccgcagcttgcctgcgcgctctgagccttcgcaactcgcgagcaaagtttggtggaggcaacgccaagcctgagtcctttcttcctctcgttccccaaatccgagggcagcccgcgggcgtcatgcccgcgctcctccgcagcctggggtacgcgtgaagcccgggaggcttggcgccggcgaagacccaaggaccactcttctgcgtttggagttgctccccgcaaccccgggctcgtcgctttctccatcccgacccacgcggggcgcggggacaacacaggtcgcggaggagcgttgccattcaagtgactgcagcagcagcggcagcgcctcggttcctgagcccaccgcaggctgaaggcattgcgcgtagtccatgcccgtagaggaagtgtgcagatgggattaacgtccacatggagatatggaagaggaccggggattggtaccgtaaccatggtcagctggggtcgtttcatctgcctggtcgtggtcaccatggcaaccttgtccctggcccggccctccttcagtttagttgaggataccacattagagccagaagagccaccaaccaaataccaaatctctcaaccagaagtgtacgtggctgcgccaggggagtcgctagaggtgcgctgcctgttgaaagatgccgccgtgatcagttggactaaggatggggtgcacttggggcccaacaataggacagtgcttattggggagtacttgcagataaagggcgccacgcctagagactccggcctctatgcttgtactgccagtaggactgtagacagtgaaacttggtacttcatggtgaatgtcacagatgccatctcatccggagatgatgaggatgacaccgatggtgcggaagattttgtcagtgagaacagtaacaacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtagtggagaatgaatacgggtccatcaatcacacgtaccacctggatgttgtggagcgatcgcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctgccagcgcctggaagagaaaaggagattacagcttccccagactacctggagatagccatttactgcataggggtcttcttaatcgcctgtatggtggtaacagtcatcctgtgccgaatgaagaacacgaccaagaagccagacttcagcagccagccggctgtgcacaagctgaccaaacgtatccccctgcggagacaggtaacagtttcggctgagtccagctcctccatgaactccaacaccccgctggtgaggataacaacacgcctctcttcaacggcagacacccccatgctggcaggggtctccgagtatgaacttccagaggacccaaaatgggagtttccaagagataagctgacactgggcaagcccctgggagaaggttgctttgggcaagtggtcatggcggaagcagtgggaattgacaaagacaagcccaaggaggcggtcaccgtggccgtgaagatgttgaaagatgatgccacagagaaagacctttctgatctggtgtcagagatggagatgatgaagatgattgggaaacacaagaatatcataaatcttcttggagcctgcacacaggatgggcctctctatgtcatagttgagtatgcctctaaaggcaacctccgagaatacctccgagcccggaggccacccgggatggagtactcctatgacattaaccgtgttcctgaggagcagatgaccttcaaggacttggtgtcatgcacctaccagctggccagaggcatggagtacttggcttcccaaaaatgtattcatcgagatttagcagccagaaatgttttggtaacagaaaacaatgtgatgaaaatagcagactttggactcgccagagatatcaacaatatagactattacaaaaagaccaccaatgggcggcttccagtcaagtggatggctccagaagccctgtttgatagagtatacactcatcagagtgatgtctggtccttcggggtgttaatgtgggagatcttcactttagggggctcgccctacccagggattcccgtggaggaactttttaagctgctgaaggaaggacacagaatggataagccagccaactgcaccaacgaactgtacatgatgatgagggactgttggcatgcagtgccctcccagagaccaacgttcaagcagttggtagaagacttggatcgaattctcactctcacaaccaatgaggaatacttggacctcagccaacctctcgaacagtattcacctagttaccctgacacaagaagttcttgttcttcaggagatgattctgttttttctccagaccccatgccttacgaaccatgccttcctcagtatccacacataaacggcagtgttaaaacatgaatgactgtgtctgcctgtccccaaacaggacagcactgggaacctagctacactgagcagggagaccatgcctcccagagcttgttgtctccacttgtatatatggatcagaggagtaaataattggaaaagtaatcagcatatgtgtaaagatttatacagttgaaaacttgtaatcttccccaggaggagaagaaggtttctggagcagtggactgccacaagccaccatgtaacccctctcacctgccgtgcgtactggctgtggaccagtaggactcaaggtggacgtgcgttctgccttccttgttaattttgtaataattggagaagatttatgtcagcacacacttacagagcacaaatgcagtatataggtgctggatgtatgtaaatatattcaaattatgtataaatatatattatatatttacaaggagttattttttgtattgattttaaatggatgtcccaatgcacctagaaaattggtctctctttttttaatagctatttgctaaatgctgttcttacacataatttcttaattttcaccgagcagaggtggaaaaatacttttgctttcagggaaaatggtataacgttaatttattaataaattggtaatatacaaaacaattaatcatttatagttttttttgtaatttaagtggcatttctatgcaggcagcacagcagactagttaatctattgcttggacttaactagttatcagatcctttgaaaagagaatatttacaatatatgactaatttggggaaaatgaagttttgatttatttgtgtttaaatgctgctgtcagacgattgttcttagacctcctaaatgccccatattaaaagaactcattcataggaaggtgtttcattttggtgtgcaaccctgtcattacgtcaacgcaacgtctaactggacttcccaagataaatggtaccagcgtcctcttaaaagatgccttaatccattccttgaggacagaccttagttgaaatgatagcagaatgtgcttctctctggcagctggccttctgcttctgagttgcacattaatcagattagcctgtattctcttcagtgaattttgataatggcttccagactctttggcgttggagacgcctgttaggatcttcaagtcccatcatagaaaattgaaacacagagttgttctgctgatagttttggggatacgtccatctttttaagggattgctttcatctaattctggcaggacctcaccaaaagatccagcctcatacctacatcagacaaaatatcgccgttgttccttctgtactaaagtattgtgttttgctttggaaacacccactcactttgcaatagccgtgcaagatgaatgcagattacactgatcttatgtgttacaaaattggagaaagtatttaataaaacctgttaatttttatactgacaataaaaatgtttctacagatattaatgttaacaagacaaaataaatgtcacgcaacttatttttttaataaaaaaaaaaaaaaa

SEQ ID NO: 10 - DNA, 713 bp, Homo sapiens
  source 1..713: mol_type = other DNA
ccccacatcggtccgtcctgcttccagctgctgcagcgcgccttcgccgccaaagcatccagcagccccctgctccggcccagcatggcgaccccgacccagacccccacaaaggctcctgaggaacctgacccattttactatgactacaacacggtgcagactgtgggcatgactctggcaaccatcttgttcctgctgggtatcctcatcgtcatcagcaagaaggtgaagtgcaggaaggcggactccaggtctgagagcccaacctgcaaatcctgtaagtctgagcttccctcttcagcccctggtggcggcggcgtgtaacaccttcccgaggaaactccgctgccgaccctgcctgagcgcgggagcctgaggaccgggtggaggcggtggggacccagccgcgcgccgggagcgctccccggaatgagccgccccacccaccccaaggctggagccgctgcaccctgctgtccctctccaggccttggcaatgacgatcccccaaagagcccgtctgcaccccagacccagggcctcaggcctccagctcctgggatccgggagtccatcccggcccagcacccccagcatccccgtgtatggcccccctgcacctccttgtctcatccccgaagatccgtccccctggcccctcagtgtccatgtcttgagcttaataaatgtgcatttggttttttcctctg

SEQ ID NO: 11 - DNA, 8518 bp, Homo sapiens
  source 1..8518: mol_type = other DNA
accctcttggtgtgcgcgtcatggccgtcagcaccgcgttcccgtcctcttccgcttggccccagaaagtttcggttctgcccggcggtggacccacgagcgcgtgccaccatggagtctgaccactgctgagcagacagccaccgagggccgaaattctgagccttcctctggacccaggcaggagacatacagacaagaaaggcaaactcaccatggcctccaccaatgcagagagccagctccagagaatcatccgagacttgcaagatgctgtgacagaactaagcaaagaatttcaggaagcaggggaacccatcacggatgacagcaccagcttgcataaattttcttataaacttgagtatctcctgcaatttgatcagaaagagaaggccaccctcctgggcaacaagaaggactactgggattacttctgtgcctgcctggccaaggtgaaaggagccaatgatgggatccgctttgtcaagtctatctcagagctccgaacatccttggggaaaggaagagcatttattcgctactccttggtgcaccagaggttggcagacaccttacagcagtgcttcatgaacaccaaagtgaccagtgactggtactatgcaagaagcccctttctgcagccaaagctgagctcggacattgtgggccaactctatgagctgactgaggttcagtttgacctggcgtcgaggggctttgacttggatgctgcctggccaacatttgccaggaggacgctgaccactggctcttctgcttacctgtggaaaccccctagccgcagctccagcatgagcagcttggtgagcagctacctgcagactcaagagatggtgtccaactttgacctgaacagccccctaaacaacgaggcattggagggctttgatgagatgcgactagagctggaccagttggaggtgcgggagaagcagctacgggagcgcatgcagcagctggacagagagaaccaggagctgagggcagctgtcagccagcaaggggagcaactgcagacagagagggagagggggcgcactgcagcggaggacaacgttcgcctcacttgcttggtagctgagctccagaagcagtgggaggtcacccaggccacccagaacactgtgaaggagctgcagacatgcctgcagggcctggagctaggagcagcagagaaggaggaggactaccacacagccctgcggcggctggagtccatgctgcagcccttggcacaggagcttgaggccacacgggactcactggacaagaaaaaccagcatttagccagcttcccaggctggctagccatggctcagcagaaggcagatacggcatcagacacaaagggccggcaagaacctattcccagtgatgcggcccaggagatgcaggagctaggggagaagcttcaagccctagaaagggagagaaccaaggtcgaggaggtcaacagacagcagagtgcccaactggaacagctggtcaaggagcttcagctgaaagaggatgcccgggccagcctggagcgcctggtgaaggagatggccccactccaggaggagttgtctgggaagggacaggaggcagaccagctctggcgacggctgcaggagttgctggcccacacgagctcctgggaggaggagctagcagagttgaggcgggagaaaaaacagcaacaggaggagaaggagctgctggagcaggaggtcaggtctctgacccggcagctgcagttcctggagacccagctggcacaggtgagccaacatgtgagtgacctggaggagcagaagaagcagctcattcaggacaaagaccacctcagccagcaggtgggtatgctcgagcggcttgctgggccgcctggcccagaactgccagtggcaggtgagaagaatgaggccctggtccctgtgaactccagtctgcaagaggcctgggggaagccagaggaggagcagaggggcctgcaggaggcacagttagacgataccaaggtgcaagagggcagccaggaggaagagctccggcaggccaacagggagctggagaaggagctacagaatgtggtcgggcgtaaccagctcctggagggcaagctgcaagccctgcaggccgattaccaggctttgcagcagcgggaatcagccatccagggctccttggcctccctggaggccgagcaggccagcatccggcacttgggtgaccagatggaggcgagcttgctggctgtaaggaaggccaaggaggccatgaaagcccagatggcagagaaggaggccattctacagagcaaggagggcgagtgtcagcagctgcgggaggaggtggagcagtgccagcaactggcagaagcccggcacagagagcttagggctctcgagagccagtgccagcagcagacccagctgattgaggtcctcacagcagagaaaggccaacagggagttggcccacccactgacaatgaagcccgtgagctggctgcccagctagccctgtctcaggcgcagctggaagtccatcagggggaggtccaacggctgcaggctcaggtggtggacctccaggccaagatgcgggcagccctggatgaccaggacaaggtgcagagccagctaagcatggctgaggccgtcctgagggagcacaaaacccttgtgcagcagctgaaggagcagaatgaagcccttaacagagcccatgtccaggagctgctgcaatgctcggagcgtgaaggggcactgcaggaggagagggccgatgaggcccagcagagggaggaggagctgcgggccctgcaggaggagctgtcccaggccaaatgcagctccgaggaagcacagctggagcacgctgagctgcaagagcagctgcaccgggccaacacagacacagctgagctgggcatccaggtttgcgcactgaccgtggaaaaggagcgagtggaggaggcactggcctgtgctgtccaggagctccaggacgccaaagaggcagcctcaagggagcgagagggcctggagcgccaagtagctgggctgcagcaagagaaggagagcttgcaggagaagctgaaggcggccaaggcagcagccggctcactgcctggcctgcaggcccagctcgcccaggcagagcagcgggcccagagcctccaagaggctgcacaccaggagctcaacaccctcaagttccagctgagtgctgaaatcatggactaccagagcagacttaagaatgctggtgaagagtgcaagagcctcaggggccagcttgaggagcaaggccggcagctgcaggctgctgaggaagctgtggagaagctgaaggccacccaagcagacatgggagagaagctgagctgcactagcaaccatcttgcagagtgccaggcggccatgctgaggaaggacaaggagggggctgccctgcgtgaagacctagaaaggacccagaaggaactcgaaaaagccacaacaaaaatccaagagtattacaacaaactctgccaggaggtgacaaatcgtgagaggaatgaccagaagatgcttgctgacctggatgacctcaacagaaccaagaagtatctcgaggagcggctgatagagctgctcagggacaaggatgctctctggcagaagtcagatgccctggaattccagcagaagctcagtgctgaggagagatggctcggagacacagaggcaaaccactgcctcgactgtaagcgggagttcagctggatggtgcggcggcaccactgcaggatatgtggccgcatcttctgttactactgctgcaacaactacgtcctgagcaagcacggtggcaaaaaggagcgctgctgccgagcctgtttccagaagctcagtgaaggccctggctcccctgatagcagtggctcaggcactagccagggagagcccagccctgcactgtcaccagcctcacctgggccccaggccacaggaggccaaggagcaaatacagactacaggccaccggacgacgctgtgtttgatatcatcacagatgaggaattgtgccagatacaggagtccggctcctctttgcctgaaacacccactgaaactgattctctcgacccaaatgcggctgaacaggatactacatcaacctcgctaacgcctgaggacactgaagacatgcccgtggggcaggattcggaaatctgcctgctgaagtctggagaactgatgatcaaagtacccctcacagtggatgagatcgccagcttcggggagggtagcagggagctgtttgtgaggtccagcacctacagcctgatccccatcactgtggccgaggcaggcctcaccatcagctgggtcttctcctctgaccccaagagcatctccttcagtgtggtcttccaggaggccgaggacacaccgctggatcagtgtaaggtcctcattcccacgacccgatgcaactcccacaaggagaacatccagggccagctcaaggttcgcacacccggcatctacatgctcatcttcgacaataccttctcaaggtttgtctctaaaaaggtattttatcacttgacggttgatcggcctgtgatctacgatggaagtgatttcctgtagcttcagcacctcagtaacttcacttcatccacaggaaacactgctcttcctcacctgtcacataaagcatttttttaaaaagtcagctgctccaaaatcatcaactcagcccctgggctgcccctcagaggcggtgtctggggaggactttgtgctcagcactctgcaccggccactcttagcccccgaggcgttgaagggctcaggcaatgtttccattaagtagagactcagctgttgtcacacccaaagggatgctctgccaaaggtttaaacacccaggagaccatcagcctctcctgggagcacagttggctacaggcctcttgtggagagtttcacgggcaggggtgattccaacttctgcctgtggagagattttctgccctgccccaccagggccctgcatgttggagactgagctgggtgcactggccatgccctgtgaatcctcaggctgtgacgccctcaggtactcctgggaaaaggaggtacacagccatcatgcgagtcggtgccaggggaccccccggagatcctgaccagctcctccagtcatgctcttgtccctcactgccccagtaagctggaggctgctccagaactcagcagtgttggaggggcctctaagctgcactctctttctggcccttttgtctgggtgattctgtcctcaaataaagcccttcactcagccagacctctccacagctcaaagcattgccctaagaatcagaagtaaagataatccaagagcaaaacccactgtacttggggcctgcaatggctgtgtgtacactacatctaatgcccaaatgccagccagtgtggatgttgtgaccacagagcaggattgtgcattggctttagagctactcctcagctgatggcccacttttgtttatataaataagagcttctgccccacctgcagacatgtttactaatgatcatagccaggattagaaccactttcaaacattggggccttcttaacaaaagtctttgataacttaagaaccaaagtaacagagtaaacagaggcatgatggatccctgggccccactcccctcctgacaggttccccaacagcccatttgcccacttcccactgctcagcccacaccagacctccaggagacatccccccttgaggcagagagatcctgttccctattcccagacaagaattatttaatcttccctgttctctgtggtccttttcttccccaacaacagatagctcaccttggacagctcttcgtcccttgttcatggaaccagctgcctgcagtcaggccccaggttcttccatgggtgaacagagcatctgacaaaaggtcccagtttggccaggggtgagggagagagcaccagacaggctatccgagaatctgagagctgggcccggcagttcctccagctacccttgtgacctaagtccagtcacacatttcccaaagtttctctttgtcataaccctggtctggctggttttgagggcttgagaatgggtcagggactccaggccaagtccaacagagaccccaaacccaccacacaccagcagccacaacctcaccaccaacaaagaggacttttgtggggccacaagtaagaggtcatttctggaatggactcagacctttaaacaggagagttgagcacttccagtcagtttttaagcaaggcatggggaacagggaatagaacctttcaaagaggttgcccagagaaaagctgggcctcttgcattcggcttccttggagcagcctcttctggcagaaagccatcaggtgctcaatcatcttctcctggccaaggctctgaccatgcttagtactggaatagaggtggccaggcccccagcgactcttcttggcctgatgtttgtcctcacaggcatgccacgtggcctgagatgattcagaacaaatcatgctaactttgaatccatccagccacttgcaaatgataatcagaagtcagcttgttcactgttagaaagaaactaacaaaagagaacccagagcaatctagaatctttgagtgcttggctttccaaggatactgcggagactctggccaagctgatgaccttctgaagtgtcactggcaccatatgcaacaagaaccaccattcactgagtagctaatgggtttggggcctgggacattccatctgaggtccttcctgaacatgtcactccacagcagaggaccggttgcagcttacccagaaccactcctccaggagagctggatgttttgcgtgcaacaccttgagcactgactgctattgttcaaaaaaagcctttgctgcattcggaggactgccccgtgccctgaggtgacttcctaactatgtggtttcattagcgaatttattttttgtgctgggtggacatttgtattttgttaggttgctgtttaagctcaagtttgctgtgctctctgcagctacaaaacatcttggcatatttaagagtggcttttataaatagctttattctgatattaatcagattcccaactttactgagaattaaggactggggtactttaaagaaatgcaaatagcaattgaagaaccactgctgcaggtggtagccctggctagactgaattacactagaaatcagccagaaggaagcgtccttgggatcccagatcactctttttttttttttttaaaaggggcagccccttgatggctcatctctctgaataacagttacgtcttcatatcgataccagatgccttcttcatcatgccactgaagccactcaccaccttcaagaacatgccaacctctgtcagattcacttacccacaaacaaggaggcacgtttggcacaaagtgttgtcctccaggtccaagtggactctacagagtgcttgacctcaacacactggattccaggtggactggaccaagagcaggcaaagacacgggaactgaaaaactccacagggtttggagaatagaaatgaaaagccacgtcatataactcaagaataaatggtgttttggaaattttaaaattatcatcgaaggtggtgaaactatttcaggcccaaatgaaaggaaatcgccagttggggatgaaatcacagagcctgtgttttatgatatggttggatgtccactgatgaaattttaaaggagtttcagttttaaaagtgcgcatgattctacatatgagaattctttaggccaagaaactgtccttggctcagaggtgttgggaattaaagcagagagaagccattcgtgatgcttagaaccaaggatggtcatgtacacaaagaccatcgagacggccattcttgtttacaaaacacttaccaagaaagcactttgtaggggaactttagtaagttcttctcatttcattatgtttcttccaaggaaacaggagagactgaattaataattctctctttcctcttaagcacttttaaaataataaagtacatcttgaaatttggggaggcatctctgatttaaaaaaagaaaaaggctgcttgatgtatgttatgcagagacactctgcctctggtggctgcagagcaatacccaagcctcatttggaaggctcaacatttggaattgcactttaattgattaatcctcaattcatgtggccttacgggatggtgggtctgggaccccaattcattcttatctgccaaagaattatctagaagcacatcaaataccagcaccccacctgctcaatgggggtggaaaacttttgtatccctaagcatattattttatagtgtctgccatgccatgtggaaatactttatttttaacctcaggatttaaataaagtaaacactatgacatttagaca

SEQ ID NO: 12 - DNA, 6789 bp, Homo sapiens
  source 1..6789: mol_type = other DNA
ctcgcgccaggcgagtctccgcgtctccctcgcgaactcggtgaaaggaattggcgccgttcgacaccaggcggatccgctctgcagcacgaacccatctccagccgcagccgcagccgccgcccgggccgaggagcagccgcagcagccgccaccagtggccgagtgagcggagccgagtttgaggcagcgcctagcggtgaatcggggccctcaccatgagttcctcgcctgttaatgtaaaaaagctgaaggtgtcggagctgaaagaggagctcaagaagcgacgcctttctgacaagggtctcaaggccgagctcatggagcgactccaggctgcgctggacgacgaggaggccgggggccgccccgccatggagcccgggaacggcagcctagacctgggcggggattccgctgggcgctcgggagcaggcctcgagcaggaggccgcggccggcggcgatgaagaggaggaggaagaggaagaggaggaggaaggaatctccgctctggacggcgaccagatggagctaggagaggagaacggggccgcgggggcggccgactcgggcccgatggaggaggaggaggccgcctcggaagacgagaacggcgacgatcagggtttccaggaaggggaagatgagctcggggacgaagaggaaggcgcgggcgacgagaacgggcacggggagcagcagcctcaaccgccggcgacgcagcagcaacagccccaacagcagcgcggggccgccaaggaggccgcggggaagagcagcggccccacctcgctgttcgcggtgacggtggcgccgcccggggcgaggcagggccagcagcaggcgggaggggacggcaaaacagaacagaaaggcggagataaaaagaggggtgttaaaagaccacgagaagatcatggccgtggatattttgagtacattgaagagaacaagtatagcagagccaaatctcctcagccacctgttgaagaagaagatgaacacttcgatgacacagtggtttgtcttgatacttataattgtgatctacattttaaaatatcaagagatcgtctcagtgcttcttcccttacaatggagagttttgcttttctttgggctggaggaagagcatcctatggtgtgtcaaaaggcaaagtgtgttttgagatgaaggttacagagaagatcccagtaaggcatttatatacaaaagatattgacatacatgaagttcgtattggctggtcactaactacaagtggaatgttacttggtgaagaagaattttcttatgggtattctctaaaaggaataaaaacatgcaactgtgagactgaagattatggagaaaagtttgatgaaaatgatgtgattacatgttttgctaactttgaaagtgatgaagtagaactctcgtatgctaagaatggacaagatcttggcgttgccttcaaaatcagtaaggaagttcttgctggacggccactgttcccgcatgttctctgccacaactgtgcagttgaatttaattttggtcagaaggaaaagccatattttccaatacctgaagagtatactttcatccagaacgtccccttagaggatcgagttagaggaccaaaggggcctgaagagaagaaagattgtgaagttgtgatgatgattggcttgccaggagctggaaaaactacctgggttactaaacatgcagcagaaaatccagggaaatataacattcttggcacaaatactattatggataagatgatggtggcaggttttaagaagcaaatggcagatactggaaaactgaacacactgttgcagagagccccccagtgtcttgggaaatttattgagattgctgcccgaaagaagcgaaattttattctggatcagacaaatgtgtctgctgctgcccagaggagaaaaatgtgcctgtttgcaggcttccagcgaaaagctgttgtagtttgcccaaaagatgaagactataagcaaagaacacagaagaaagcagaagtagaggggaaagacctaccagaacatgcggtcctcaaaatgaaaggaaactttaccctcccagaggtagctgagtgctttgatgaaataacctatgttgaacttcagaaggaagaagcccaaaaactcttggagcaatataaggaagaaagcaaaaaggctcttccaccagaaaagaaacagaacactggctcaaagaaaagcaataaaaataagagtggcaagaaccagtttaacagaggtggtggccatagaggacgtggaggattcaatatgcgtggtggaaatttcagaggaggagcccctgggaatcgtggcggatataataggaggggcaacatgccacagagaggtggtggcggtggaggaagtggtggaatcggctatccataccctcgtgcccctgtttttcctggccgtggtagttactcaaacagagggaactacaacagaggtggaatgcccaacagagggaactacaaccagaacttcagaggacgaggaaacaatcgtggctacaaaaatcaatctcagggctacaaccagtggcagcagggtcaattctggggtcagaagccatggagtcagcattatcaccaaggatattattgaatacccaaataaaacgaactgatacatatttctccaaaaccttcacaagaagtcgactgttttctttagtaggctaactttttaaacattccacaagaggaagtgcctgcgggttccttttttagaagctttgtgggttgattttttttcttttcttttttgtacatttttaattgcagtttaaaagtgaatcgtaagagaacctcagcattgtgcacgataagagaatgtgtcagtatttcagggttctacattttatctgtaaaatgtgacttttttttttttttatcacaacagaagtaaaatgttgctttgtacctggtgtcttttattaagaatttactccccccatttctcacagagaataacagtcgggagtcattgtcacaatataatagaaatgttagcaaccagattcatgtaaggactaagtggtcctcatgaattgcattaagactctgtactgctcatattacactccatcctctctgtagtttgctgggtagtggagggggtaagctaaatcatagtttctgacaataactgggaaggttttttcttaaaataacaatggaattggtataattgggattgaaaactaaaacttggaactaagatagagaagatggagtgtatgtagaagggctgttaaaaatgtaaaacttggttgcattatttgtggaggctcaaacttgtgaaggttaataccataatttttccatttgttctgcattttgattctgaaaagaaagctggctttgcccatttcttattaaaaaaacttgttgtaaatccagttgtctaatgggatctatatgaagttagccatgtctgtatgcccttctcccacaaaatactgtataactagtgtgcttgtagtagttaactccaccatctttgtaagctaatgaaattgtgagtcacccatttatatcttaatttttaatcatgtcagttcttgaatgggtatctccttagcctgctgatttctttttctttctaaagaaagtgggtggagaaattaatttagacgtttgtttgcaataaaaagaattcattttactcttgttttgggattctcgccatcaaggttcaaaatccctttatataactcccaagaggagaaatttattaagtgtgtgctttctggacagcttattctttactctgcatagaacatttaggttttaaaaacttaaatgtatactgacaattgatacataattatgaagtaaagttgaattcttcccttcccctcccccccagacaacttttaacatatttaatgaggggaaaaggtactggctgggagaagttaacactgagtttatcatctttacagaatgctaatgctgtcctcaactgattattttatatacatatatatgatacatgaaactctgggatcagatgcttttagaagccatcatgcaagccagtcattgatgtcactgctacacaacactgctaacttgactgtagctatgtaataacattagatcccctaattgtaattatattgggtttgcacagaacactttaatcttcccctcaccaatgtgaagtgaggaatcaggagtcaaactgtagaactaaaatttgacttcagtctagcgtttccttggtgtttttaggttgctttggtaagtttaggtttgctatatttctgattgcttagaattttgttttagccctttaaaatcagatcataaatatgaattcatacttctaaggaattttcttgctataagctggagtttaggtgatgtataggttcagttgagacatttttggaacaggcaaatccttagttaacataagatatttaacagttgaagatagtgtcatggatttttatcttttttagcaagtaatgctaagaaccactggcctgagctactactcttcagtatacattattaggattgcatagacttactagaggaacagtttcaggttttgatgctaatcagtgttgtgtcctaaagttgtcctttgtgcctttaaaaagttttggatatatcttctagtttaaaattgcttattaaggaattcattttataattgcagtgggaaagtaatggtcaagtaacactaggtagactatcatgcctgtttagcccagagaatttggggggagagagaatagataaaaatggcacccagaaaaatgttaaaatctttagtcaagactagaattaatacaattgtctacacttgtatggcagaaataaccttataaagtgtttaaggaattcagagaagggaatgtaccaaataagcaacagggagaaaattaggtaagaagtaagatacgaacgagaaacctgatttattgctcatccttcccttgcctccctaatggcaagcaaaactctgaacatctgaaaaggatgtagttctggacaaatcctgactacccagaggaaactcactgtgagattgctgttgatttgaagggtgctttcactaaggttatattttaaagtagaataacacatgctgagtgtaaactggctttggattggtcagctgcagtagtacaaaaacagcatagaatttgagaaaactaaaactgctatgagatagctatgagaaaactaaaactgctatgagatagaaatgatgtaaaattatgtggaaagttttccctcatatactcacatacagcctttgaagggctctggctctgaccggttgatggccttgagcgagatgaaatcatgaaattgagtcaaatcaatttgacattgaaatgacaagaggaaactcttaaatacataaaaacaagctctcatttgcctaggatagatactgtcttaaaaataaagactgaacctagatgttctgagcactagcaacaaggtattttaacaagtttaaaggaattctctgaaaaagttataaaattattctaggaaacataaccataatagtgttttaagggactttcacctggggattttatattcatgaacagagtgtattctgtatttaaaatgtctcatttgtgggaattggatgacatgttttttgataaatttattcacaatataaattgactttttattctaggaccatgtgaataatgggttccattgcacaaatacaaatattttaatagcttcttaggcagtggtgtagacatcttggatataaataattgtagatcttgtatatttgatttttaaaaaactagaataaacagagaggcataaacatatcttagagtccaagtggtagtgtttagcattggatataataaatggatgttttacaaagtgtttccataattctcttcctatacataaatgtcttgttttcaaaagtggatggaacttggctgggtgtggtggctcacgcctgtaatcctagcactttgggaggccaaggcgggaggatcacttgagctcaggagtttgagaccatcctgggcaacatagtgagacctggtctcttgaaaaaaaaaaagtggatggaacttgtagcagagaattttatctacttctcaacctgcttcagaatacccatttgagatgttcccctggaaagatgaacacaatactgcatctgaagccatttcttcccacctaacattcttaaaatgattagagtctaaactttgtcattcattcctaattctggagctctgaggttgaggtgttcagagtttggtgaataattgggtttaagttttaacattttagtaataataaaagcaaacacatacatgttaaggcctgacaataggttgcaatacccatgcattgggactcatacccagcataaatggtgagggactgaacattagtgctttgagcaagaattggttaactacctgagatctctaaaacagtaatttgaattactaatacataaaccacaagtctcttcgaattgttaattgcctaaatttaccctaaaacttagttaacgctgcagtgcctttattaaagcttttttttgggggggggtgtgggcacagctactccataactttaaattttaaagcatgaaggtgatctagctagtgttagtgcttgagttggtagttacataggctgctcttgaatcgtcctgttttctttgcctagtattagcaccacaggtacatatttgtaaaataagcattaaaagtactttgtccagc

SEQ ID NO: 13 - DNA, 1809 bp, Homo sapiens
  source 1..1809: mol_type = other DNA
tcgagcccgctttccagggaccctacctgagggcccacaggtgaggcagcctggcctagcaggccccacgccaccgcctctgcctccaggccgcccgctgctgcggggccaccatgctcctgcccaggcctggagactgacccgaccccggcactacctcgaggctccgcccccacctgctggaccccagggtcccaccctggcccaggaggtcagccagggaatcattaacaagaggcagtgacatggcgcagaaggagggtggccggactgtgccatgctgctccagacccaaggtggcagctctcactgcggggaccctgctacttctgacagccatcggggcggcatcctgggccattgtggctgttctcctcaggagtgaccaggagccgctgtacccagtgcaggtcagctctgcggacgctcggctcatggtctttgacaagacggaagggacgtggcggctgctgtgctcctcgcgctccaacgccagggtagccggactcagctgcgaggagatgggcttcctcagggcactgacccactccgagctggacgtgcgaacggcgggcgccaatggcacgtcgggcttcttctgtgtggacgaggggaggctgccccacacccagaggctgctggaggtcatctccgtgtgtgattgccccagaggccgtttcttggccgccatctgccaagactgtggccgcaggaagctgcccgtggaccgcatcgtgggaggccgggacaccagcttgggccggtggccgtggcaagtcagccttcgctatgatggagcacacctctgtgggggatccctgctctccggggactgggtgctgacagccgcccactgcttcccggagcggaaccgggtcctgtcccgatggcgagtgtttgccggtgccgtggcccaggcctctccccacggtctgcagctgggggtgcaggctgtggtctaccacgggggctatcttccctttcgggaccccaacagcgaggagaacagcaacgatattgccctggtccacctctccagtcccctgcccctcacagaatacatccagcctgtgtgcctcccagctgccggccaggccctggtggatggcaagatctgtaccgtgacgggctggggcaacacgcagtactatggccaacaggccggggtactccaggaggctcgagtccccataatcagcaatgatgtctgcaatggcgctgacttctatggaaaccagatcaagcccaagatgttctgtgctggctaccccgagggtggcattgatgcctgccagggcgacagcggtggtccctttgtgtgtgaggacagcatctctcggacgccacgttggcggctgtgtggcattgtgagttggggcactggctgtgccctggcccagaagccaggcgtctacaccaaagtcagtgacttccgggagtggatcttccaggccataaagactcactccgaagccagcggcatggtgacccagctctgaccggtggcttctcgctgcgcagcctccagggcccgaggtgatcccggtggtgggatccacgctgggcctaggatgggacgtttttcttcttgggcccggtccacaggtccaaggacaccctccctccagggtcctctcttccacagtggcgggcccactcagccccgagaccacccaacctcaccctcctgacccccatgtaaatattgttctgctgtctgggactcctgtctaggtgcccctgatgacgggatgctctttaaataataaagatggttttgattaaaaaaaaaaaaaaaaaaaaaaaaaa

SEQ ID NO: 14 - DNA, 1861 bp, Homo sapiens
  source 1..1861: mol_type = other DNA
cactcaaggtgtgcaggcagctgtgtttgtcaggaaggcagaaggagttggctttgctttaggggaggagacgaggtcccacaacaccctctgaagggtatataaggagccccagcgtgcagcctggcctggtacctcctgccagcatctcttgggtttgctgagaactcacgggctccagctacctggccatgaccaccacatttctgcaaacttcttcctccacctttgggggtggctcaacccgagggggttccctcctggctgggggaggtggctttggtggggggagtctctctgggggaggtggaagccgaagtatctcagcttcttctgctaggtttgtctcttcagggtcaggaggaggatatgggggtggcatgagggtctgtggctttggtggaggggctggtagtgttttcggtggaggctttggagggggcgttggtgggggttttggtggtggctttggtggtggcgatggtggtctcctctctggcaatgagaaaattaccatgcagaacctcaatgaccgcctggcctcctacctggacaaggtacgtgccctggaggaggccaatgctgacctggaggtgaagatccatgactggtaccagaagcagaccccaaccagcccagaatgcgactacagccaatacttcaagaccattgaagagctccgggacaagatcatggccaccaccatcgacaactcccgggtcatcctggagatcgacaatgccaggctggctgcggacgacttcaggctcaagtatgagaatgagctggccctgcgccagggcgttgaggctgacatcaacggcttgcgccgagtcctggatgagctgaccctggccaggactgacctggagatgcagatcgagggcctgaatgaggagctagcctacctgaagaagaaccacgaagaggagatgaaggagttcagcagccagctggccggccaggtcaatgtggagatggacgcagcaccgggtgtggacctgacccgtgtgctggcagagatgagggagcagtacgaggccatggcggagaagaaccgccgggatgtcgaggcctggttcttcagcaagactgaggagctgaacaaagaggtggcctccaacacagaaatgatccagaccagcaagacggagatcacagacctgagacgcacgatgcaggagctggagatcgagctgcagtcccagctcagcatgaaagctgggctggagaactcactggccgagacagagtgccgctatgccacgcagctgcagcagatccaggggctcattggtggcctggaggcccagctgagtgagctccgatgcgagatggaggctcagaaccaggagtacaagatgctgcttgacataaagacacggctggagcaggagatcgctacttaccgcagcctgctcgagggccaggatgccaagatggctggcattggcatcagggaagcctcttcaggaggtggtggtagcagcagcaatttccacatcaatgtagaagagtcagtggatggacaggtggtttcttcccacaagagagaaatctaagtgtctattgcaggagaaacgtcccttgccactccccactctcatcaggccaagtggaggactggccagagggcctgcacatgcaaactccagtccctgccttcagagagctgaaaagggtccctcggtcttttatttcagggctttgcatgcgctctattccccctctgcctctccccaccttctttggagcaaggagatgcagctgtattgtgtaacaagctcatttgtacagtgtctgttcatgtaataaagaattacttttccttttgcaaataaaaaaaaaaaaaaaaaaaaaa

SEQ ID NO: 15 - DNA, 1700 bp, Homo sapiens
  source 1..1700: mol_type = other DNA
aactagcacgtggctcccgcccccagactgcttctttattcattcctaacctttaccttggcagatgaaatataagattcatcaaccacatttgacagcccatggcaggtttcctgttttccatcgtccctctgcaggtcacagacacacagagcccagccgtggcaggctcagccggggtccggggctgctaacaacggctacattcctcccccagggccaagggaaatcctgagcgcaggccagggttgtttggttttgaggtgtgctgggatgaaaggcaccctggaagtggaaggttcggtcattcattaattaattacatctataattgagggtttgttcttaagagcgagtcctttgaaagtactttccttcaaacagtgactgccacaaaggcatcagatattcaccaccttctcggctgcctcagcacagcaagctttattctgggacctgagatcctgttctgagctggctttcccttctccaggctcgctcaccctccctttagagatagtggatggtaagatgaccaatgctcagattattcttctcattgacaatgccaggatggcagtggatgacttcaacctcaagtatgaaaatgaacactcctttaagaaagacttggaaattgaagtcgagggcctccgaaggaccttagacaacctgaccattgtcacaacagacctagaacaggaggtggaaggaatgaggaaagagctcattctcatgaagaagcaccatgagcaggaaatggagaagcatcatgtgccaagtgacttcaatgtcaatgtgaaggtggatacaggtcccagggaagatctgattaaggtcctggaggatatgagacaagaatatgagcttataataaagaagaagcatcgagacttggacacttggtataaagaacagtctgcagccatgtcccaggaggcagccagtccagccactgtgcagagcagacaaggtgacatccacgaactgaagcgcacattccaggccctggagattgacctgcagacacagtacagcacgaaatctgctttggaaaacatgttatccgagacccagtctcggtactcctgcaagctccaggacatgcaagagatcatctcccactatgaggaggaactgacgcagctacgccatgaactggagcggcagaacaatgaataccaagtgctgctgggcatcaaaacccacctggagaaggaaatcaccacgtaccgacggctcctggagggagagagtgaagggacacgggaagaatcaaagtcgagcatgaaagtgtctgcaactccaaagatcaaggccataacccaggagaccatcaacggaagattagttctttgtcaagtgaatgaaatccaaaagcacgcatgagaccaatgaaagtttccgcctgttgtaaaatctattttcccccaaggaaagtccttgcacagacaccagtgagtgagttctaaaagatacccttggaattatcagactcagaaacttttattttttttttctgtaacagtctcaccagacttctcataatgctcttaatatattgcacttttctaatcaaagtgcgagtttatgagggtaaagctctactttcctactgcagccttcagattctcatcattttgcatctattttgtagccaataaaactccgcactagctgcaaaaaaaaaaaaaaaaaaa

SEQ ID NO: 16 - DNA, 4048 bp, Homo sapiens
  source 1..4048: mol_type = other DNA
ggaagtgggcctggcaccttcccggcctgccgcagggatggggcagctgtgctggctgccgctgctggcaccgctcctgttgctgcgaccgccaggggtccagtccgccggccccatccgggccttcgtggtgccccacagccacatggacgtgggctgggtctacactgtgcaggaaagcatgcgggcgtacgccgccaatgtctacacctcagtggtggaagagctggcccgcggccagcagcgccggttcatcgctgtggagcaggagtttttccggctgtggtgggatggcgtcgcctcggaccagcagaaataccaggtccgccagctcctggaggaaggacgcctggaatttgtcatcggaggccaggtcatgcatgacgaggctgtgacgcaccttgatgaccagatcctgcagctcacagaaggacacgggtttctctatgaaacatttgggatccggccacagttctcctggcacgttgacccgtttggcgcctctgccacgacgcccaccctatttgcgctggcgggcttcaatgcccacctcggctcccggatcgactacgacctgaaggcagccatgcaggaggcccgggggctgcagttcgtgtggcgagggtccccatccctctcagagcggcaggaaatcttcacgcacatcatggaccagtacagctactgcaccccgtcccacatccctttctccaacaggtcaggattttactggaatggcgtggctgtcttccccaagcctccccaagatggggtgtaccccaacatgagtgagcctgtcaccccagccaacatcaacctctatgccgagctcggtgtctcggtgcagtatgccacgctgggcgactacttccgtgccctgcacgctctcaatgtcacctggcgtgtccgcgaccaccacgacttcctgccctattccacagaaccattccaggcctggacgggcttctacacgtcccgcagctcactgaaggggctggcccggcgagccagcgccttgttgtatgccggggagtccatgttcacacgctacctgtggccggccccccgtgggcatctggaccccacctgggccctgcagcagctccagcagcttcgctgggccgtctccgaggtccagcaccatgatgccatcactgggactgagtcccccaaggtgagagacatgtacgcaacgcacctggcctcggggatgctgggcatgcgcaagctgatggcctccatcgtcctagatgagctccagccccaggcacccatggcggccagctccgatgcaggacctgcaggacattttgcctcggtctacaacccgctggcctggacggtcaccaccatcgtcaccctgactgttggtttccctggagtccgcgtcacagatgaggcgggccacccagtgccctcgcagatccagaactcaacagagaccccatctgcgtatgacctgcttattctgaccacaatcccaggcctcagttaccggcactacaacatcagacccactgcaggggcccaagagggcacccaggagccggctgccactgtggcgagcacccttcaatttggccgcaggctgaggagacgcaccagccatgcgggcaggtacttggtgcctgtggcaaacgactgctacattgtgctgctcgaccaggataccaacctgatgcacagcatctgggagagacagagtaaccgaacggtgcgcgtgacccaggaattcctggagtaccacgtcaacggggatgtgaaacagggccccatttccgataactacctgttcacaccgggcaaggccgcggtgcctgcgtgggaagctgtggaaatggagattgtggcgggacagcttgtgactgagatccggcagtacttctacaggaacatgacagcacagaattacacgtatgcaatccgctcccggctcacccatgtgccgcagggccatgacggggagctgctctgccaccggatagagcaggagtaccaagccggccccctggagctgaaccgtgaggctgtcctgaggaccagcaccaacctaaacagccagcaggtcatctactcagacaacaacggctaccagatgcagcggaggccctacgtttcctatgtgaacaacagcatcgcccggaattactaccccatggttcagtcggccttcatggaggatggcaaaagcaggcttgtgttgctgtcggagcgggcacatggcatctccagccaagggaatgggcaggtggaggtcatgctccaccggcggctgtggaacaacttcgactgggacctgggctacaacctcacgctgaacgacacctcagtcgtccacccagtgctctggcttctgctgggatcctggtccctcaccactgccctgcgccagaggagcgcactggcgctgcagcacaggcccgtggtgctgttcggagacctcgctgggactgcgccgaagctcccaggaccccagcagcaagaggccgtgacgctgcccccgaatcttcacctgcagatcctgagcatccctggctggcgctacagctccaaccacacggagcactctcagaatctccggaaaggccatcgaggggaagcccaggctgacctccgccgtgtcctgctgcggctctaccacctgtatgaagtgggcgaggacccagtcctgtctcagccagtgacagtgaatctggaggctgtgctgcaggcgctggggtccgtggtggcagtggaggagcgctcgctcacagggacctgggatttgagcatgctgcaccgctggagctggaggacggggcctggccgccacagaggtgacaccacctctccctcgaggccaccaggaggccccatcatcaccgtccacccaaaggaaatccggacgttctttattcactttcaacagcagtgagccctgggcagatgccccggccccagggcttcccccaggaactccatgtaacagaacagacccaggacagggaaaagcagtgcggagggatgggactggggagtcagctgctcatctgcaggctaatggcaggaaatggtcatatttggggtttttccctaatttttttaaacaaaaattacattacaagatccaggttcttcccccccacactcaatcaagccagccctctcctcttctgtcacgtaaaggatatttggcacactcatgcgtcattcattcacaaaacacaaacccaggactttctgcctaaggcagagcacaagactcacagcagcaccgaagcgcatctgccgtccgggccctgccaggcttgccaggctgccagtggtaactgtggacctactgcgtgccacgtgttttcatagactcatcccatgctggcaacagccctgcaaggggcttggctctgccacagggcaggagaggaagttgtagcgcctagcgagagttccagccccagacgcccacctgtgcctcagggcaccgcctgccgagcagagaaggcacagcagccgtcagagtccatgagaggtgaaaccacacagcagggatgtccaatatcagaactattaatatcaataaaagtataaccttcccaggtctatgcccaagagaattgaaaacatccatccacacaatacctgtgctcccgcgttcatagcagcattactcaaaagtcaaacggtagcaacaacccaaatgtccatccacagatgaattaagacatgaagtgtgttctgtccatacaatggaatattatttggccataaaaaggaaggaaattctgacgcatgccacagcctgagtgaatcctacaaatattacgctaagtgaaagaagccaatcacgagtttatgtgaaatgtccagaataggcaaatctgtgtatcagagacaaagcacattggtggttgccaggtactggaggaagagagaagaggcatgacagctaacagggacgggctttctttggaagatgatgaaattgtggaatgatggttgcacaactttgtgaatatactagaaaccaatgaattaaaaactttggaagatgaaaaaaaaaaaaaaaaaa

SEQ ID NO: 17 - DNA, 2141 bp, Homo sapiens
  source 1..2141: mol_type = other DNA
tgcgtgccgccgttgatccggtgctgcagtgaggaggtggttcttgcccgtgttgtgtgtgtgtgtgagtgagagagcgagtgagtgagtgagtgagtgtgtgtgtgggggggactcggcttgttgttgtcggtgacttccccctccccttcaccccttcccctccccgccgccgctgcagtggccgctccctgggccgtaggaaatgagcgataacgatgacatcgaggtggagagcgacgaagagcaaccgaggtttcaatctgcggctgacaaacgggctcatcataatgcactggaacgaaaacgtagggaccacatcaaagacagctttcacagtttgcgggactcagtcccatcactccaaggagagaaggcatcccgggcccaaatcctagacaaagccacagaatatatccagtatatgcgaaggaaaaaccacacacaccagcaagatattgacgacctcaagcggcagaatgctcttctggagcagcaaggggaaagcgagagctgatcaagttctttgttcctggggaattcacttctcttcctccctcatggaagatgcaagtaaaaggaaattccgtgcactggagaaggcgaggtcaagtgcccaactgcagaccaactacccctcctcagacaacagcctctacaccaacgccaagggcagcaccatctctgccttcgatgggggctcggactccagctcggagtctgagcctgaagagccccaaagcaggaagaagctccggatggaggccagctaagccactcggggcaggccagcaataaaaactgtctgtctccatcgtctcatcctcctttcagttcgttggtagagccctcagaaccatttaagagactctttatttttctctttctcccttttttttttaaatttttatttttacgtagaagctcttggacaacagctctcgttctccttccccatttccactgtatattttttaatgtattcccttcagggattccctgtccccaacaggaatttttaaaccaaaacaccccaacttggcagctttttctgtggaggacagacggccggccggacctctgagcacatagtgtcctgcccaccctaccagctcctccagccctgccgggcacatgcccgggggacgcctgccctgcccaggtggcctcctggcccgccctcacctctgatagactttgtgaatctgaactgctctactttgagaagatgaccggtttggagtaatcagaatgaaccctcctcctttttaagggttttttttttttcctttttctaaaaagctatgtatcgctcctattgaaagaccagatccttagagaagtttgtggtataaaaaggaagtggggacagattcgcagcacagagtcgctggcatgtttcactcctgcttctctcagccagctgtttaagcctgcggcgccagcctcacggagggccgtgtgacactctcgtggtatgtatgggagatggcagcagtgaagcagcagccaccagggagtggccatttggggttgggacagggagggtgttttgggtggcatagaggttttgtattgagggccagtgatgatgttttgatatttatttcctgctacttaaatttgaatctgagtgaattgtacctatttctgatgatgtcggtcttgcaaagcgacagattcataaagtaatgatgaaatctttctttcttcccgtgtgtatttctaagaaatagagccaactgattttgtatgtaaataccaagagcaatttacctggtactaaacccgcaccccagtgcggacccttcccagccctcatcccacttcctttcctactgtcctggaacctgtctccattgtgtgatccagccctggttctggctgtggtcagcagatgccagtgaagggttttgtgtgtttaggcctcatttctttgtctttttcctactccgttcctggcatttgctgatttctagtgtatactctgtagtctcagttcgtgtttgattccattccatggaaataaaaagtatgttgtacatactgccgaagaattgtcttgcaagttaaggcttccccctttactataagactataaataaaaacttattttatccttcttaaaaaaaaaaaaaaaa

SEQ ID NO: 18 - DNA, 4602 bp, Homo sapiens
  source 1..4602: mol_type = other DNA
gaagcgtctcctgtgcgtctgcgcgggaagtggaacctggctctggggagaagccgcgtgagatccgcgcgggtgctagctagtcctttctcgtcgctgctcggctcgcggcccgtggggtcggccccgccaccgttgccgccatgcccatgaagggccgcttccccatccgccgcaccctgcaatatctgagccaggggaacgtggtgttcaaggactccgtgaaggtcatgacagtgaattacaacacgcatggggagctgggcgagggcgccaggaagtttgtgtttttcaacatacctcagattcaatacaaaaacccttgggtgcagatcatgatgtttaagaacatgacgccgtcacccttcctgcgattctacttagattctggggagcaggtcctggtggatgtggagaccaagagcaataaggagatcatggagcacatcagaaaaatcttggggaagaatgaggaaaccctcagggaagaggaggaggagaaaaagcagctttctcacccagccaacttcggccctcgaaagtactgcctgcgggagtgcatctgtgaagtggaagggcaggtgccctgccccagcctggtgccattacccaaggagatgagggggaagtacaaagccgctctgaaagccgatgcccaggactaaggcccacggtcactgtgggctggggtgatggtgtctgaccagtggggagattggaatgggattactttggcccagggaagcccctggttctgtccctggagactctggaaatccttttgcattaaaaggactttacacacctgtgtaaaaggatgtgggagaggagggtctgaagctgagctgctaaatgaatatccctgctctgctggtcaataaaacgcttcctaatagcagcttggcgtgtatctggtcctagtgaagaggaaggcctgtgtagcagaaaggctttgggcctgagaggttaaggccacagcctgttgacacctgttttggtcctgcgaccctttactggtctccgctggctttgaatcttcctctgggctctactctggagaacataagggctgctgtggttgagtctggctagcactgtctgtggttggcagtgtgtacacccctccgttcagttccttgggggtatttttcagaaatccaaaggcaacccttcgtgcagtgctcacttttttaagtacagttgattacccttgcctgctggggggcctagccatgggccagagatggaggagccccagtggctgacaggccagcctcactcaggcacgtacctgctgaccagtcagccactgccaacccatggcccagccactgtgtgcattagcagggaggtttgtaggccatggaggaaatgaggagacaccacctagtggagacattggggccctgctggggggatggtgtctatagctggctctgctggctccctcaggccctgcttaccaagctctggaggagggagtgctgcattactgagcaccttccttgttctttcctcataggacactgatgttactgtcactttagttatgctaaagtggaggtttcagcctccagaagacagcagagccttctagggtcaccttaagaataggtttagctaggctgggtgtggtggctcatgcctgtaatcccagcactttgggaggctgatgtgggcggatagttgagcccaggagttcgagaccagcctgggcaacatggcaagaccttgtctctacaaaaagtacaaaaattagatgggtgtggtgtttcgcgtctgtagtcccagctatttgggaagctgaggtgggaggatctcgagcctgcgagatcaaggctgcagtgaatcatgatcgtgcgctactgcactccagcctgggccacagagcaagactgtctcaagaaaaaaaattttttaaataagtttaattataaagtgaaggaccagttggaccacggacccctagaaactcagccaaggagacctgactttatctgagacaggaaggcagtggttaaaagaattaaaaaaggacagtggtgcttgacagagcaggactgaagttttcattcctctacctgctggggccttggtcaagtcctaaaagctttccaggtctgtaactggttttgtctgtaaaatggggaaaaggttaccttttctgccacatgaagtagctggctggtgaaatgatgtcatactacgatgtgtttcattaactgttggagattatcacattaaattgagcagatttggatcttttacatttgatgctcaagtttgattctgtaccagatctgggtttggggtgatggcttgtcgggggtgggaggggaaggtgggcagagactgatgtataatggacaggtgttccttgagtgacagacttacaaagggagtgtttaattgaaatctagcatcttgtctctctgctatcagcattttggctgggtggaaaaagctctcctccacagagagccctcccatgtctgctggtttcagttccccacccaactgactgaagtcttgaccttgataccacctgcctgggcgttctacttggctgcaagggctcttcagaaagcacctagttccttttggctttcatctgcagtgtaggtctccatagctagtgacacacaaagccttatgagagtattccttccctctagggcccaaagaggctgctaaccagcaggaatgtcctgaaccaaaaggagtggtgaccattctctagacagaataaatgtctggtccctaatgggacagataactagggtttgagttcaggcttgacacaggagcaatgggcagtgaggtggacagcccagaccctgaaaggacgtgtggagacagcagcagtcctgtggtgcagttactgcctgtgtggccagcttcacaggcagcagataatttgtggaaataaacattattttctttttttgttccctgtaggctacttgatcctggatagtttctctttaaacctttgtctttccctattttgtttagtagtgatgaaagatgggtttaaaatatacagaaatgggagaaaattaattagagaaaatgtgaggattttataattgataacatgttcttaattttacttgcacaagcagtgttcacagccttattctgaagtgaccacttttcttctctttgaggcactacctggcagcaccatctaaacctgtatcatctgctgtggtcccagttactcaggaggctgaggcaggaggatcacttgagcccaggaggttgaggctggagtgagccatgactgtaccactgcattccagcctgggtgacggagtgaggccctatctgcctgccagtgagttctgtcatgcatcagactttggacctcagaattttcacctggaacatgagaggtcagatgacataatgaatatgttctaacttctgaaacttttggagcttgtttttgcccttgatagcacgtatccttagagaataagcttgccaatttaaggcctgtctgtaatggcatctgaatgttccagcagcctaactgtgctctgctccagaaacatgtgtgtggcttccctacctccacaccaaggagctgccttgctgggtggatgtaaagactgtcctgccagggtttcccactaaaggtattaataagtccagtggaccacagagggcccaatccctcttggccgctaaggctaaggtaggagccagagagctgtagagacaggaccaccaggatgcttcatgtccataacacaagagagcgctgcattgtttgggggccaggcattactactatcatgaagaaaaatgaagagcaggatgcatgttagtctttaattcacttttaatagtataaacctcatttaggtagtagtattaagccacaacaataatgccacattgaaacagcatttaataaaatgcataaagctaattcatgcactgcaatactctatatacaaacacaacaatgcaaattcttcttcaagactgaagacattgattagatataaaattcagtttaaaaagaacatgctattttttaaatgccatcacataaacaaagtgatttcacagggagaagaaagctgtataaagctgcagctttcaacaggttttaaacctggcattaaaatgtaatggcaaaaccaatattttctatattgtgaagggcaaaggttacagaaacggtcccaagaaaatctaacacccaaattttcctttgatagatagatgcctttaaaagggctggtaatgcagttacattctaacagagaagtccaaactacaggtaaaaactacggcttgtactgtgaaaaatgtgcagcttttcagttataaaactagttgaacactggtttacaagataatccgtagaacagagagactgtagaaaaatattccagcacttgagttgtgtgtggcagcagcatttgagtccatgaatgctatggtcattaaaataattgattatactttcctaaagaaaagccattttttaatgcaaagtctactacttccaaatgttattccaaacattaaattttaacagtctatcaatcaataaatgttgaacattttttttctaaaaaaaaa

SEQ ID NO: 19 - DNA, 2082 bp, Homo sapiens
  source 1..2082: mol_type = other DNA
aaaagccagccaggagctgtgggaggaaacgccctcagtaaagatgaccgcggtcactgttatctaaacgcaagtgaagccgagtcacaggacccggatgttgtcagttcgacggtaaacgaccctgccagcttccaagagggcggcttcactgtgcgaataggtgagaagccaagaaggaggcgcgctggagttacttccgcccggttctccttcccgcagtctgcagccggagtaagatggcggcgctgagggctttgtgcggcttccggggcgtcgcggcccaggtgctgcggcctggggctggagtccgattgccgattcagcccagcagaggtgttcggcagtggcagccagatgtggaatgggcacagcagtttgggggagctgttatgtacccaagcaaagaaacagcccactggaagcctccaccttggaatgatgtggaccctccaaaggacacaattgtgaagaacattaccctgaactttgggccccaacacccagcagcgcatggtgtcctgcgactagtgatggaattgagtggggagatggtgcggaagtgtgatcctcacatcgggctcctgcaccgaggcactgagaagctcattgaatacaagacctatcttcaggcccttccatactttgaccggctagactatgtgtccatgatgtgtaacgaacaggcctattctctagctgtggagaagttgctaaacatccggcctcctcctcgggcacagtggatccgagtgctgtttggagaaatcacacgtttgttgaaccacatcatggctgtgaccacacatgccctggaccttggggccatgacccctttcttctggctgtttgaagaaagggagaagatgtttgagttctacgagcgagtgtctggagcccgaatgcatgctgcttatatccggccaggaggagtgcaccaggacctaccccttgggcttatggatgacatttatcagttttctaagaacttctctcttcggcttgatgagttggaggagttgctgaccaacaataggatctggcgaaatcggacaattgacattggggttgtaacagcagaagaagcacttaactatggttttagtggagtgatgcttcggggctcaggcatccagtgggacctgcggaagacccagccctatgatgtttacgaccaggttgagtttgatgttcctgttggttctcgaggggactgctatgataggtacctgtgccgggtggaggagatgcgccagtccctgagaattatcgcacagtgtctaaacaagatgcctcctggggagatcaaggttgatgatgccaaagtgtctccacctaagcgagcagagatgaagacttccatggagtcactgattcatcactttaagttgtatactgagggctaccaagttcctccaggagccacatatactgccattgaggctcccaagggagagtttggggtgtacctggtgtctgatggcagcagccgcccttatcgatgcaagatcaaggctcctggttttgcccatctggctggtttggacaagatgtctaagggacacatgttggcagatgtcgttgccatcataggtacgaggcctattgtgtagtagaggtatcctagacaaaggagttcgggacgcccactggggacagaaggagaacacttcctgttcaccataggccatggcatggactcgggtcctcaatcttttgagcacagtaatgggttctggatcttgggtaacaccactttttttgtttgttttgcctcacaacaggaagataagtaacatcacttttttcctccatcctctcacctaggtacccaagatattgtatttggagaagtagatcggtgagcaggggagcagcgtttgatcccccctgcctatcagcttcttctgtggagcctgttcctcactggaaattggcctctgtgtgtgtgtgtgtgtgtgtgtgtgtgtgtatgttcatgtacacttggctgtcaggctttctgtgcatgtactaaaaaaggagaaattataataaattagccgtcttgcggcccctaggcctaaaaaaaaaaaaaaaaaa

SEQ ID NO: 20 - DNA, 6318 bp, Homo sapiens
  source 1..6318: mol_type = other DNA
tagtaagacaggtgccttcagttcactctcagtaaggggctggttgcctgcatgagtgtgtgctctgtgtcactgtggattggagttgaaaaagcttgactggcgtcattcaggagctggatggcgtgggacatgtgcaaccaggactctgagtctgtatggagtgacatcgagtgtgctgctctggttggtgaagaccagcctctttgcccagatcttcctgaacttgatctttctgaactagatgtgaacgacttggatacagacagctttctgggtggactcaagtggtgcagtgaccaatcagaaataatatccaatcagtacaacaatgagccttcaaacatatttgagaagatagatgaagagaatgaggcaaacttgctagcagtcctcacagagacactagacagtctccctgtggatgaagacggattgccctcatttgatgcgctgacagatggagacgtgaccactgacaatgaggctagtccttcctccatgcctgacggcacccctccaccccaggaggcagaagagccgtctctacttaagaagctcttactggcaccagccaacactcagctaagttataatgaatgcagtggtctcagtacccagaaccatgcaaatcacaatcacaggatcagaacaaaccctgcaattgttaagactgagaattcatggagcaataaagcgaagagtatttgtcaacagcaaaagccacaaagacgtccctgctcggagcttctcaaatatctgaccacaaacgatgaccctcctcacaccaaacccacagagaacagaaacagcagcagagacaaatgcacctccaaaaagaagtcccacacacagtcgcagtcacaacacttacaagccaaaccaacaactttatctcttcctctgaccccagagtcaccaaatgaccccaagggttccccatttgagaacaagactattgaacgcaccttaagtgtggaactctctggaactgcaggcctaactccacccaccactcctcctcataaagccaaccaagataacccttttagggcttctccaaagctgaagtcctcttgcaagactgtggtgccaccaccatcaaagaagcccaggtacagtgagtcttctggtacacaaggcaataactccaccaagaaagggccggagcaatccgagttgtatgcacaactcagcaagtcctcagtcctcactggtggacacgaggaaaggaagaccaagcggcccagtctgcggctgtttggtgaccatgactattgccagtcaattaattccaaaacagaaatactcattaatatatcacaggagctccaagactctagacaactagaaaataaagatgtctcctctgattggcaggggcagatttgttcttccacagattcagaccagtgctacctgagagagactttggaggcaagcaagcaggtctctccttgcagcacaagaaaacagctccaagaccaggaaatccgagccgagctgaacaagcacttcggtcatcccagtcaagctgtttttgacgacgaagcagacaagaccggtgaactgagggacagtgatttcagtaatgaacaattctccaaactacctatgtttataaattcaggactagccatggatggcctgtttgatgacagcgaagatgaaagtgataaactgagctacccttgggatggcacgcaatcctattcattgttcaatgtgtctccttcttgttcttcttttaactctccatgtagagattctgtgtcaccacccaaatccttattttctcaaagaccccaaaggatgcgctctcgttcaaggtccttttctcgacacaggtcgtgttcccgatcaccatattccaggtcaagatcaaggtctccaggcagtagatcctcttcaagatcctgctattactatgagtcaagccactacagacaccgcacgcaccgaaattctcccttgtatgtgagatcacgttcaagatcgccctacagccgtcggcccaggtatgacagctacgaggaatatcagcacgagaggctgaagagggaagaatatcgcagagagtatgagaagcgagagtctgagagggccaagcaaagggagaggcagaggcagaaggcaattgaagagcgccgtgtgatttatgtcggtaaaatcagacctgacacaacacggacagaactgagggaccgttttgaagtttttggtgaaattgaggagtgcacagtaaatctgcgggatgatggagacagctatggtttcattacctaccgttatacctgtgatgcttttgctgctcttgaaaatggatacactttgcgcaggtcaaacgaaactgactttgagctgtacttttgtggacgcaagcaatttttcaagtctaactatgcagacctagattcaaactcagatgactttgaccctgcttccaccaagagcaagtatgactctctggattttgatagtttactgaaagaagctcagagaagcttgcgcaggtaacatgttccctagctgaggatgacagagggatggcgaatacctcatgggacagcgcgtccttccctaaagactattgcaagtcatacttaggaatttctcctactttacactctctgtacaaaaacaaaacaaaacaacaacaatacaacaagaacaacaacaacaataacaacaatggtttacatgaacacagctgctgaagaggcaagagacagaatgatatccagtaagcacatgtttattcatgggtgtcagctttgcttttcctggagtctcttggtgatggagtgtgcgtgtgtgcatgtatgtgtgtgtgtatgtatgtgtgtggtgtgtgtgcttggtttaggggaagtatgtgtgggtacatgtgaggactgggggcacctgaccagaatgcgcaagggcaaaccatttcaaatggcagcagttccatgaagacacgcttaaaacctagaacttcaaaatgttcgtattctattcaaaaggaaatatatatatatatatatatatatatatatatatatataaattaaaaaggaaagaaaactaacaaccaaccaaccaaccaaccaaccacaaaccaccctaaaatgacagccgctgatgtctgggcatcagcctttgtactctgtttttttaagaaagtgcagaatcaacttgaagcaagctttctctcataacgtaatgattatatgacaatcctgaagaaaccacaggttccatagaactaatatcctgtctctctctctctctctctctctctcttttttttttctttttccttttgccatggaatctgggtgggagaggatactgcgggcaccagaatgctaaagtttcctaacattttgaagtttctgtagttcatccttaatcctgacacccatgtaaatgtccaaaatgttgatcttccactgcaaatttcaaaagccttgtcaatggtcaagcgtgcagcttgttcagcggttctttctgaggagcggacaccgggttacattactaatgagagttgggtagaactctctgagatgtgttcagatagtgtaattgctacattctctgatgtagttaagtatttacagatgttaaatggagtatttttattttatgtatatactatacaacaatgttcttttttgttacagctatgcactgtaaatgcagccttcttttcaaaactgctaaatttttcttaatcaagaatattcaaatgtaattatgaggtgaaacaattattgtacactaacatatttagaagctgaacttactgcttatatatatttgattgtaaaaacaaaaagacagtgtgtgtgtctgttgagtgcaacaagagcaaaatgatgctttccgcacatccatcccttaggtgagcttcaatctaagcatcttgtcaagaaatatcctagtcccctaaaggtattaaccacttctgcgatatttttccacattttcttgtcgcttgtttttctttgaagttttatacactggatttgttaggggaatgaaattttctcatctaaaatttttctagaagatatcatgattttatgtaaagtctctcaatgggtaaccattaagaaatgtttttattttctctatcaacagtagttttgaaactagaagtcaaaaatctttttaaaatgctgttttgttttaatttttgtgattttaatttgatacaaaatgctgaggtaataattatagtatgatttttacaataattaatgtgtgtctgaagactatctttgaagccagtatttctttcccttggcagagtatgacgatggtatttatctgtattttttacagttatgcatcctgtataaatactgatatttcattcctttgtttactaaagagacatatttatcagttgcagatagcctatttattataaattatgagatgatgaaaataataaagccagtggaaattttctacctaggatgcatgacaattgtcaggttggagtgtaagtgcttcatttgggaaattcagcttttgcagaagcagtgtttctacttgcactagcatggcctctgacgtgaccatggtgttgttcttgatgacattgcttctgctaaatttaataaaaacttcagaaaaacctccattttgatcatcaggatttcatctgagtgtggagtccctggaatggaattcagtaacatttggagtgtgtattcaagtttctaaattgagattcgattactgtttggctgacatgacttttctggaagacatgatacacctactactcaattgttcttttcctttctctcgcccaacacgatcttgtaagatggatttcacccccaggccaatgcagctaattttgatagctgcattcatttatcaccagcatattgtgttctgagtgaatccactgtttgtcctgtcggatgcttgcttgattttttggcttcttatttctaagtagatagaaagcaataaaaatactatgaaatgaaagaacttgttcacaggttctgcgttacaacagtaacacatctttaatccgcctaattcttgttgttctgtaggttaaatgcaggtattttaactgtgtgaacgccaaactaaagtttacagtctttctttctgaattttgagtatcttctgttgtagaataataataaaaagactattaagagcaataaattatttttaagaaatcgagatttagtaaatcctattatgtgttcaaggaccacatgtgttctctattttgcctttaaatttttgtgaaccaattttaaatacattctcctttttgccctggattgttgacatgagtggaatacttggtttcttttcttacttatcaaaagacagcactacagatatcatattgaggattaatttatcccccctacccccagcctgacaaatattgttaccatgaagatagttttcctcaatggacttcaaattgcatctagaattagtggagcttttgtatcttctgcagacactgtgggtagcccatcaaaatgtaagctgtgctcctctcatttttatttttatttttttgggagagaatatttcaaatgaacacgtgcaccccatcatcactggaggcaaatttcagcatagatctgtaggatttttagaagaccgtgggccattgccttcatgccgtggtaagtaccacatctacaattttggtaaccgaactggtgctttagtaatgtggatttttttcttttttaaaagagatgtagcagaataattcttccagtgcaacaaaatcaattttttgctaaacgactccgagaacaacagttgggctgtcaacattcaaagcagcagagagggaactttgcactattggggtatgatgtttgggtcagttgataaaaggaaaccttttcatgcctttagatgtgagcttccagtaggtaatgattatgtgtcctttcttgatggctgtaatgagaacttcaatcactgtagtctaagacctgatctatagatgacctagaatagccatgtactataatgtgatgattctaaatttgtacctatgtgacagacattttcaataatgtgaactgctgatttgatggagctactttaagatttgtaggtgaaagtgtaatactgttggttgaactatgctgaagagggaaagtgagcgattagttgagcccttgccgggccttttttccacctgccaattctacatgtattgttgtggttttattcattgtatgaaaattcctgtgattttttttaaatgtgcagtacacatcagcctcactgagctaataaagggaaacgaatgtttcaaatcta

SEQ ID NO: 21 - DNA, 4598 bp, Homo sapiens
  source 1..4598: mol_type = other DNA
gtagttcctttcccacaatcggctgggcgaggcggcgccagcgatcagagcagcgctgggtgttcaggggccaagatggcggcgcgccggggacggagagacggagtcgcgccgcccccgagtgggggccccggtccggaccctggcgggggagcccgcggcagtggttggggaagtcgaagccaagcgccgtatgggactttgggcgctgtgagcggcggcgagcaggtgctgctgcatgaggaggcgggtgattctggctttgtcagtctctctcggctgggcccatctctgagggacaaggacctggaaatggaggagctaatgctgcaggatgagacactgctggggaccatgcagagctacatggatgcctcccttatctccctcattgaggattttgggagccttggagagagcaggttatctctggaggaccagaatgaagtgtcgctgctcacggctctgacggagatcttggacaatgcagattctgagaacctttctccatttgacagcattcctgattcggagctgcttgtgtcaccccgggagggctcctctctgcacaagctgcttactctctctcggacacccccagaacgtgacctcatcaccccagttgacccactggggcccagtacaggcagcagtagagggagtggggttgaaatgtctcttccagatccctcttgggacttctccccaccctctttcttagagacctcttcccccaagcttcctagctggagacccccaagatcaagaccacgctggggccaatccccacctccccagcagcgcagtgatggagaagaagaggaggaggtggccagcttcagtggccagattcttgccggggagcttgacaactgtgtgagcagtatcccggacttccccatgcatttggcctgccctgaggaggaagataaagcaacagcagcagagatggcagtgccagcagctggtgatgagagcatctcctccctgagtgagctggtgcgggccatgcacccatactgcctgcccaacctcacccacctggcatcacttgaggatgagcttcaggagcagccagatgatttgacactgcctgagggctgcgtagtgctggagattgtggggcaggcagccacagctggcgatgacctggagatcccagttgtggtgcgacaggtctctcctggaccccggcctgtgctcctggatgactcgctagagactagttctgccttgcagctgcttatgcctacactggagtcagagacagaggctgctgtgcccaaggtaaccctctgctctgagaaagaggggttgtcattgaactcagaggagaagctggactcagcctgcttattgaagcccagggaggtcgtggagccggtggtgcccaaggagcctcagaacccacctgccaatgcagcaccaggttcccagagagctcgaaagggcaggaagaagaagagcaaggagcagccagcagcctgtgtggaaggctatgccaggaggctgaggtcatcttctcgcgggcagtctactgtaggtacagaagtgacctctcaggtagacaacttgcagaaacagcctcaggaagaacttcaaaaagagtctgggcctctccagggtaaggggaagccccgggcttgggctcgggcctgggcagctgccttggagaattctagccctaagaacttggagagaagtgctggacaaagtagtcctgctaaagaaggccctctagacctctacccaaagctggctgacactatccaaaccaatcctataccaacccatctctcattggtcgactctgcccaagccagccccatgccagttgactctgttgaagctgatcccactgcagttggccctgttctagctggccctgtacctgttgaccctgggttggttgaccttgcttcaaccagctcagaactggttgagcctctcccggctgagccagtgctgatcaacccagtcctggctgactcagcagcagttgaccctgcagtggttcccatctcagataacttgccaccagttgatgctgtcccgtctggcccagcaccagttgatctagcactggttgaccctgttcctaatgacctgactccagttgacccagtgctagttaagtccagaccaactgatcccagacgtggtgcagtgtcatcagccctggggggttcagcaccccagctcctcgtggagtcagagtccttggacccaccaaagaccatcatccctgaagtcaaagaggttgtggattctctgaaaattgaaagtggtaccagtgctacaacccatgaagccagacctcggcctctcagcttatctgagtaccggcgacgaaggcagcaacgccaagcagaaacagaagagagaagtccacagcccccaactgggaagtggcctagccttccagagactcccacagggctggcagacatcccttgtcttgtcatcccaccagccccagccaagaagacagctctgcagagaagccctgaaacaccccttgagatttgccttgtgcctgtaggtcccagccctgcttctcctagtcctgagccacctgtaagcaaacctgtggcctcatctcccactgagcaggtgccatcccaggagatgccactgttggcgagaccttcccctcctgtgcagtctgtgtcccctgctgtgcccacacctccctcgatgtctgctgccctgcctttccctgcaggtgggcttggcatgccccccagtctgcccccacctcccttgcagcctcctagtcttccattgtctatggggccagtactacctgatccgtttactcactatgcccccttgccatcctggccttgttatcctcatgtgtccccttctggctatccttgcctgccccccccaccaacggtgcccctagtgtctggtactcctggtgcctatgccgtgcctcccacttgcagtgtgccttgggcaccccctcctgccccagtctcaccttacagttccacatgtacctatgggcccttgggatggggcccagggcctcaacatgctccattctggtctactgttcccccacctcctttgcctccagcctccattgggagagctgttccccaacctaaaatggagtctaggggcactccagctggccctcctgaaaatgtacttcccttgtcgatggctcctcccctcagtcttgggctacctggccatggagctcctcagacagagcctaccaaggtggaggtcaagccagtgcctgcatctccccatccgaaacacaaggtgtctgccctggtgcaaagtccccagatgaaggctctagcatgtgtgtctgctgaaggtgtgactgttgaggagcctgcatcagagaggctaaagcctgagacccaagagaccaggcccagggagaagccccccttgcctgctaccaaggctgttcccacaccaaggcagagcactgtccccaagctgcctgctgtccacccagcccgtctaaggaagctgtccttcctgcctaccccacgtactcagggttctgaagatgtggtacaggctttcatcagtgagattggaattgaggcatcggacctgtccagtctgctggagcagtttgagaaatcagaagccaaaaaggagtgtcctcctccggctcctgctgacagcttggctgtaggaaactcagggtccagctgtagttcctctggacgttctcgaagatgctcttcctcttcttcgtcatcatcttcctcttcgtcttcctcatcctcatcatccagttctcgaagccgctcacgatccccatccccccgccggagaagtgacaggaggcggcggtacagctcttatcgttcacatgaccattaccaaaggcaaagagtgctacaaaaggagcgtgcaatagaagaaagaagggtggtcttcattggaaagatacctggccgcatgactcgatcagagctgaaacagaggttctccgtttttggagagattgaggagtgcaccatccacttccgtgtccaaggggacaactacggcttcgtcacttatcgctatgctgaggaggcatttgcagccattgagagtggccacaagctgcggcaggcagatgagcagccctttgatctctgctttgggggccgaaggcagttctgcaagaggagctattctgatcttgactccaaccgggaagactttgacccagcacctgtaaagagcaaatttgattctcttgactttgacacattgttgaaacaggcccagaagaacctcaggaggtaaccttgggcccttccctgctatcctttttctcctttggaggtgcccaacctcctccacccccttcccctactctaggggagagagctgctagtgagatgactgttttataaagaaatggaaaaaagtgaaataaaaaatatgttgaatcagattttttaaaaggggtatttgtttttttataacaggtattgaaacaagttaacttgcattcctatgtaagataggaggggctgaggggatccccagtgtttggaacataagtcactatgcagactaataaacatcaactagagagaactcccaaaaaaaaaaaaaaaaaa

SEQ ID NO: 22 - DNA, 1818 bp, Homo sapiens
  source 1..1818: mol_type = other DNA
ggtggtcagcgcgcgcattgcctgccccggaagtggtcggcgcgcggcgcggcgcgcctgggcgctaagatggcggcggcgtgagttgcatgttgtgtgaggatcccggggccgccgcgtcgctcgggccccgccatggccgtcaccatcacgctcaaaacgctgcagcagcagaccttcaagatccgcatggagcctgacgagacggtgaaggtgctaaaggagaagatagaagctgagaagggtcgtgatgccttccccgtggctggacagaaactcatctatgccggcaagatcttgagtgacgatgtccctatcagggactatcgcatcgatgagaagaactttgtggtcgtcatggtgaccaagaccaaagccggccagggtacctcagcacccccagaggcctcacccacagctgccccagagtcctctacatccttcccgcctgcccccacctcaggcatgtcccatcccccacctgccgccagagaggacaagagcccatcagaggaatccgcccccacgacgtccccagagtctgtgtcaggctctgttccctcttcaggtagcagcgggcgagaggaagacgcggcctccacgctagtgacgggctctgagtatgagacgatgctgacggagatcatgtccatgggctatgagcgagagcgggtcgtggccgccctgagagccagctacaacaacccccaccgagccgtggagtatctgctcacgggaattcctgggagccccgagccggaacacggttctgtccaggagagccaggtatcggagcagccggccacggaagcaggagagaaccccctggagttcctgcgggaccagccccagttccagaacatgcggcaggtgattcagcagaaccctgcgctgctgcccgccctgctccagcagctgggccaggagaaccctcagcttttacagcaaatcagccggcaccaggagcagttcatccagatgctgaacgagccccctggggagctggcggacatctcagatgtggagggggaggtgggcgccataggagaggaggccccgcagatgaactacatccaggtgacgccgcaggagaaagaagctatagagaggttgaaggccctgggcttcccagagagcctggtcatccaggcctatttcgcgtgtgaaaaaaatgagaacttggctgccaacttcctcctgagtcagaactttgatgacgagtgatgccaggaagccaggccaccgaagcccccaccctacccttattccatgaaagttttataaaagaaaaaatatatatatattcatgtttatttaagaaatggaaaaaaaaatcaaaaatcttaaaaaaacaagcaaacagtccagcttcctgtcctcctaaagtggcccctgttcccatctcccgggccagacagctgtccccccgtcctcctccccagcccagcctgctcagagaagctggcaggactgggaggcgacagatgggcccctcttggcctctgtcccagctctctgcagccagacggaaaggcggctgcttgcctctccatcctccgaaaaacccctgaggacccccccccatcctcttctaggatgaggggaagctggagccccaactttgatcctccattggagtggcccaaatctttccatctagggcaagtcctgaaaggcccaaggccccctccccagtctggccttggcctccagcctggagaagggctaacatcagctcattgtcaaggccacccccaccccagaacagaaccgtgtctctgataaaggttttgaagtgaataaagttttaaaaactag

SEQ ID NO: 23 - DNA, 3310 bp, Homo sapiens
  source 1..3310: mol_type = other DNA
cccggcggggggaggttggggacgggcctggcagttgtgaactcgaacctgccgctgtcgccgcggcggggcggggagcgagagtgggccgcggaggccggccttcgggctccatggacgggcgccgcgtccctgcacagcccgccgcagaggtaaggctggcctctctgcagtcagaggtctgagctctgccatggggataggggtgtctttattactgcagttttctctaacacctgggggctaccggagtgtgggccgaagcaggcgctgcagccgcggaagtatccccaggaacatccccaagaggagctggaaaaagcctcatccccagctctgcagtctccaggggagctcagtgtctgtttgtccagcttctcagagttgctgtgcagctcggatgtggcataggaaacagcagacacagggagagggcagcataaggcactgtagggagcagtggccacattttctgcagaggaagaaccgatgctggaacgtcgttgcaggggccccctggccatgggcctggcccagccccgactcctttctgggccctcccaggagtcaccccagaccctggggaaggagtcccgcgggctgaggcaacaaggcacgtcagtggcccagtctggtgcccaagccccaggcagggcccatcgctgtgcccactgtcgaaggcacttccctggctgggtggctctgtggcttcacacccgccggtgccaggcccggctgcccttgccctgccctgagtgtggccgtcgctttcgccatgcccccttcttagcactgcaccgccaggtccatgctgctgccaccccagacctgggctttgcctgccacctctgtgggcagagcttccgaggctgggtggccctggttctgcatctgcgggcccattcagctgcaaagcggcccatcgcttgtcccaaatgcgagagacgcttctggcgacgaaagcagcttcgagctcatctgcggcggtgccaccctcccgccccggaggcccggcccttcatatgcggcaactgtggccggagctttgcccagtgggaccagctagttgcccacaagcgggtgcacgtagctgaggccctggaggaggccgcagccaaggctctggggccccggcccaggggccgccccgcggtgaccgccccccggcccggtggagatgccgtcgaccgccccttccagtgtgcctgttgtggcaagcgcttccggcacaagcccaacttgatcgctcaccgccgcgtgcacacgggcgagcggccccaccagtgccccgagtgcgggaagcgctttaccaataagccctatctgacttcgcaccggcgcatccacaccggcgagaagccctacccgtgcaaagagtgcggccgccgcttccggcacaaacccaacctgctgtctcacagcaagattcacaagcgatccgaggggtcggcccaggccgcccccggcccggggagcccccagctgccagccggcccccaggagtccgcggccgagcccaccccggcggtacctctgaaaccggcccaggagccgccgccaggggccccgccagagcacccgcaggacccgatcgaagcccccccctccctctacagctgcgacgactgcggcaggagcttccggctggagcgcttcctgcgggcccaccagcggcagcacaccggggagcggcccttcacctgcgccgagtgcgggaagaacttcggcaagaagacgcacctggtggcgcactcgcgcgtgcactccggcgagcggcccttcgcctgcgaggagtgcggccgccgcttctcccagggcagccatctggcggcgcatcggcgcgaccacgcccccgatcggcccttcgtgtgtcccgactgcggcaaggccttccgccacaaaccctacctggcggcgcaccggcgcatccacaccggcgagaagccctacgtctgccccgactgcggcaaagccttcagccagaagtccaacctggtgtcgcaccggcgcatccacacgggcgagcggccctacgcctgtcccgactgcgaccgcagcttcagccagaagtccaacctcatcacccaccgcaagagccacatccgggacggcgccttctgctgtgccatctgtggccagaccttcgacgacgaggagagactcctggcccaccagaagaagcacgatgtctgagacggtgggcggggccgtgttggctgagagagggctggggtccttcgtggtgggagtcgcagtgggctgggggtgcctgcctagtgctggagtaggggacaatgggaatcctagaggggatggaagacgcggggagtgagctgggtgggccctgctagcgagagaggtcaaccccggtggccagggaacccacttccaagcgcagggacgccggcctccagctggtgtgtgctaaggctccgtcctgactgccctgtgccctggaaaagcagcaatagcatccgccccttagagccctctggctagaggagccaccagtggaaaggaagaccctccatcctctggtattaacgccttaatgcccctgtcttttactgtaagttacttaagatcatttttggaagcaggcgtggtagagtcctgtaaatgaatgctctgggctagatacagcttggagaacctgctggccttgttagacagcacttgggcctttgccagcagcaagaggtgaagcgaagccactcttacctctcccttcccctcccacctgccccctgcgtaggcacccagacttggagagacccgtctgctgttaatacttccatcctcttccttcccaaagagcagatcccaaggcatttactccttggtctgtctcgctttatctgtcgcccctcccagcgctgagagcctcccctggctgtcagcagcactgtgtccaggctcttgtctgaacaccgcagcccctccttcgctccttccagagctcagcatgtcacggcaaggactgccgcattggtgatggagggccagctgaggggaagttgctggtgagtttccttttctccatttctagcatatggacacctggcctctgcttgagcacttaggtgacaggaacttccgcacctcctgaggccctggatgattctaattgttagaaattctaattgttagaaatccttccttataatgaatgaattctgctttcctataatttctacctattgggccttgttctgttctctggaactaaacagaacaaccatttacccctccttttcaaactagagaataaagatttggttttagaactggtaaaaaaaaaaaaaaaaaa

SEQ ID NO: 24 - DNA, 1247 bp, Homo sapiens
  source 1..1247: mol_type = other DNA
actttacgcaggcgcgttaggtcctagtcgctatgcgtgtgcttgtgggtggcgggacaggcttcattgggacagccctaacccagctgctgaatgccagaggccacgaagtgacgttggtctcccgaaagcccgggcccggccggatcacgtgggatgagctcgctgcatcggggctgccgagctgcgatgccgccgtcaacctggccggagagaacatcctcaaccctctccgaagatggaatgaaaccttccaaaaagaggtaatcggcagccgcctagagaccacccaattgctggctaaagccatcaccaaagccccacaaccccccaaggcctgggtcttagtcacaggtgtagcttactaccagcccagtctgactgcggagtatgatgaagacagcccaggaggggactttgactttttctccaacctcgtaaccaaatgggaagctgcagccaggcttcctggagattctacacgccaggtggtggtgcgctcaggggttgtgctgggccgtgggggtggtgccatgggccacatgctgctgccctttcgcctgggcctggggggccccatcggctcaggccaccaattcttcccctggatacacatcggggacctggcaggaatcctgacccatgcccttgaagcaaaccacgtgcacggggtcctgaatggagtggctccatcctccgccactaatgctgagtttgcccagaccttgggtgctgccctgggccgccgagccttcatccctctccccagcgctgtggtgcaagctgtctttgggcgacagcgtgccatcatgctgctggagggccagaaggtgatcccacagcgaacactggccactggctaccagtattccttcccagagctaggggctgccttaaaggaaattgtagcctaagtaggtcgtggcaagggcctgaggcctgttcctcacaggcttccaggttaggcactgtgaataggctcagctcctctagagagctgaagccatctggttcttagattcctctcccagtcctctttcccattgttctgttgctccaccttattgtctcaaggccgtaatctcatcaggttgggacattaatcttttcaactccttgtaagatttcccagtttggtttctctacatgtcctgcagctgccccacttctcctttacgctgtgtagagaatgctctgcagtttaggcaataaaaataaattgtctcactaaaaaaaaaaaaaaaaaa

SEQ ID NO: 25 - DNA, 1804 bp, Homo sapiens
  source 1..1804: mol_type = other DNA
aagcgcggggccggccggggcgcccgccgctcgccgccgcctccgcgcgcccgggggccccggcgccccccgagctagggcggccagctcgcggctcgccgtttgacagatgctcatcgccatggagttgccgcagcagcacctttgggggctcgggcgagcgacgggagccgggatctgagcgagcgccggggccagcggagccggagccgccgggacatggttgcagatctgatctcttctgaacacctcatcgtgtctccatccctgggaatctgaccctagcaactggaccactttgttcttggaattttgggtgtcctcttttctcacctttcccttttcccttttccccttccccctcctgagaactccggaagactgtagagattgtcatggagtccagggaaaccttaagcagctcccggcaaagagggggcgagtcagacttcctgccggtctcctcagccaagcccccagctgctcctggctgtgcaggagaacctttgctctccactccaggacctgggaaggggatcccggtgggcggagagcgcatggagccagaggaggaggatgaactaggctcagggcgggatgtggattccaactccaacgcggacagtgagaaatgggtggcaggagatggtttggaagagcaggaattttctatcaaggaggcaaacttcacagagggaagtctgaagctaaagattcagaccacaaagcgggctaagaaacccccaaagaatttggagaactatatatgtccacctgagatcaagatcaccatcaagcagtctggggaccagaaggtgtcccgtgctggaaaaaatagcaaagccacgaaggaggaagaaagaagccactccaaaaagaagctcctcacagccagtgaccttgcagccagtgacctcaaaggatttcagccacagattaaagactccagtaaggaggaagtctggaagagaagaggaggccaaggcatcccattcaaaaagcaattcctgtcccaggaacgtgccatgtgcttctcatgcccccggaacccattccccgcaaaacccggttctctcactcttccttttcacagtgaacctgcagtctgggcacaagaagtataacttcgcatggattctgcaaagcccacacctgtggtcattccctgttctttccattcaacaatggagacttgcccaagattgtaaactagtgagtgacagcatttgggcttatgatcttctctgcctgctagctagacattctctctgggtctaaaagataatccaaaaagatccagcttcacaatgctgccctgaagagataatgcattaggcggccctgatgcagcattcactgtcttccagggcaggcttgatcccatgagtttgcttgctcagacgatcacttaggaaacacatgcctttactctctaggccttttttctagcttgccttgtatcagctatgccatggatctttgctctcttaccccatgctatacagagtatgggctccaagccacagctggcctgtcaagtgtgtgtcgctggtccaccatgggatacatttagaaacttttatagcaatttgacatttttgtgatatccaagcatgtgattgttttcctacggatttgtcttatagtattttaccaaagtttccacacaaaaagtatggattaaggacaaagtatctggtccttcatcaaagatcgtttgataagctctgttctagttaaccaacactgagctttcctagttttaataaaagagtaggatttggaaaaaaaaaaaaaaaaaaaa

SEQ ID NO: 26 - DNA, 4129 bp, Homo sapiens
  source 1..4129: mol_type = other DNA
acacagagcagagtggggctctgagtatataactgttaggtgcctccctccagcaccatctcctgagaagcactctcccttgtcgtggaggtgggcaaatctttatcagccactgccttctgctgccaggaagccagctagagtggtctttaaagaaaactgggcatctcctgctacttaaaatcaaaaactacctaaaataaagattataaaaaagtaaggatgaatggacggtctttgattggcggcgctggtgacgcccgtcatggtcctgtttggaaggacccttttggaactaaagctggtgacgcagcgcgcagaggcatcgcccggctaagcttggccctggcagatgggtcgcaggaacaggagccagaggaagagatagccatggaggacagccccactatggttagagtggacagccccactatggttaggggtgaaaaccaggtttcgccatgtcaagggagaaggtgcttccccaaagctcttggctatgtcaccggtgacatgaaagaacttgccaaccagcttaaagacaaacccgtggtgctccagttcattgactggattctccggggcatatcccaagtggtgttcgtcaacaaccccgtcagtggaatcctgattctggtaggacttcttgttcagaacccctggtgggctctcactggctggctgggaacagtggtctccactctgatggccctcttgctcagccaggacaggtcattaatagcatctgggctctatggctacaatgccaccctggtgggagtactcatggctgtcttttcggacaagggagactatttctggtggctgttactccctgtatgtgctatgtccatgacttgcccaattttctcaagtgcattgaattccatgctcagcaaatgggacctccccgtcttcaccctccctttcaacatggcgttgtcaatgtacctttcagccacaggacattacaatccattctttccagccaaactggtcatacctataactacagctccaaatatctcctggtctgacctcagtgccctggagttgttgaaatctataccagtgggagttggtcagatctatggctgtgataatccatggacagggggcattttcctgggagccatcctactctcctccccactcatgtgcctgcatgctgccataggatcattgctgggcatagcagcgggactcagtctttcagccccatttgaggacatctactttggactctggggtttcaacagctctctggcctgcattgcaatgggaggaatgttcatggcgctcacctggcaaacccacctcctggctcttggctgtgccctgttcacggcctatcttggagtcggcatggcaaactttatggctgaggttggattgccagcttgtacctggcccttctgtttggccacgctattgttcctcatcatgaccacaaaaaattccaacatctacaagatgcccctcagtaaagttacttatcctgaagaaaaccgcatcttctacctgcaagccaagaaaagaatggtggaaagccctttgtgagaacaagccccatttgcagccatggtcacgagtcatttctgcctgactgctccagctaacttccagggtctcagcaaactgctgtttttcacgagtatcaactttcatactgacgcgtctgtaatctgttcttatgctcattttgtattttcctttcaactccaggaatatccttgagcatatgagagtcacatccaggtgatgtgctctggtatggaatttgaaaccccaatggggccttggcactaagactggaatgtatataaagtcaaagtgctccaacagaaggaggaagtgaaaacaaactattagtatttattgatattcttggtgtttagctggctcgatgatgttaacagtattaaaaattaaaccccataaaccaactaagccttatggaattcacagtcacaaaatcgaagttaatccagaattctgtgataagcagcttggctttttttttaaatcaatgcaagttacacattatagccagaatctgtatcacagaggtgcaagctgacagcagagctcagtccccacttcctgcaaacaatggcctgcaccctatcccttgtgtgtgtgacattctctcatgggacaatgttggggtttttcagactgacaggactgcaagagggagaaaggaattttgtcaatcaaaattattctgtattgcaacttttctcagagattgcaaaggattttttaggtagagattatttttccttatgaaaaatgatctgttttaaatgagataaaataggagaagttcctggcttaacctgttcttacatattaaagaaaagttacttactgtatttatgaaatactcagcttaggcatttttactttaacccctaaattgattttgtaaatgccacaaatgcatagaattgttaccaacctccaaagggctctttaaaatcatattttttattcatttgaggatgtcttataaagactgaaggcaaaggtcagattgcttacgggtgttatttttataagttgttgaattccttaatttaaaaaagctcattattttttgcacactcacaatattctctctcagaaatcaatggcatttgaaccaccaaaaagaaataaagggctgagtgcggtggctcacgcctgtaatcccagcactttggggagcccaggcgggcagattgcttgaacccaggagttcaagaccagcctgggcagcatggtgaaaccctgtatctacaaaaaatacaaaaattagccaggcatggtggtgggtgcctgtagttccagctacttgggaggctgaggtgggaaaatgacttgagcccaggaggaggaggctgcagtgagctaagattgcaccactgcactccaacctgggcgacaagagtgaaactgtgtctctcaaaaaaaaaaaaaaacaaacaaaaacaaaaacaaaacaaaacaaaacaaaacaaaacaggtaaggattcccctgttttcctctctttaattttaaagttatcagttccgtaaagtctctgtaaccaaacatactgaagacagcaacagaagtcacgttcagggactggctcacacctgtaatcccagcactttgggagatggaggtaaaaggatctcttgagcccaggagttcaagaccagcttgggcaacatagcaagactccatctcttaaaaaataaaaatagtaacattagccaggtgtagcagcacacatctgcagcagctactcaggaggctgaggtggaaagatcgcttgtgcacagaagttcgaggctgcagtgagctatatgatcatgtcactgcactccagcctgtgtgaccgagcaagaccctatctcaaaaaaattaattaattaattaattaattaatttaaaaaggaagtcatgttcatttactttccacttcagtgtgtatcgtgtagtattttggaggttggaaagtgaaacgtaggaatcctgaagattttttccacttctagtttgcagtgctcagtgcacaatatacattttgctgaatgaataaacagaaatagggaagtaaacctacaaatattttagggagaagctcacttcttccttttctcaggaaaccaagcaagcaaacatatcgttccaattttaaaacccagtgaccaaagcctttggaactatgaatttgcaactgtcataggtttatggatattgctgtggagaagctcaattttcagtgtttgaactgaaccctttcttgttagggaacgtgtgaaagaagaattgtggggaaaaaaaagcaagcataaccaaagatcatcagcagtgaagaatctaggctgtggctgagagaaccagaggcctctaaaatggacccgagtcgatcttcagaacagggatctaccatgcaggagcttcttgtgctcacacaaatctgtaaatgggaacattgtacattgtcgaatttaaatgatattaattttctcaagctatttttgttactattttcctaaaattgaatatttgcagggagcacttatactttttcctaatgtctgtataacaaatttctatgcaagtacatgaataaattatgctcacagctca

SEQ ID NO: 27 - DNA, 3885 bp, Homo sapiens
  source 1..3885: mol_type = other DNA
agagccggacggggtaaactgagcggcggcggcggggcgctggggcggagactgcgacccggagccgcccggactgacggagcccactgcggtgcgggcgttggcgcgggcacggaggacccgggcaggcatcgcaagcgaccccgagcggagccccggagccatggccctgagcgagctggcgctggtccgctggctgcaggagagccgccgctcgcggaagctcatcctgttcatcgtgttcctggcgctgctgctggacaacatgctgctcactgtcgtggtccccatcatcccaagttatctgtacagcattaagcatgagaagaatgctacagaaatccagacggccaggccagtgcacactgcctccatctcagacagcttccagagcatcttctcctattatgataactcgactatggtcaccgggaatgctaccagagacctgacacttcatcagaccgccacacagcacatggtgaccaacgcgtccgctgttccttccgactgtcccagtgaagacaaagacctcctgaatgaaaacgtgcaagttggtctgttgtttgcctcgaaagccaccgtccagctcatcaccaaccctttcataggactactgaccaacagaattggctatccaattcccatatttgcgggattctgcatcatgtttgtctcaacaattatgtttgccttctccagcagctatgccttcctgctgattgccaggtcgctgcagggcatcggctcgtcctgctcctctgtggctgggatgggcatgcttgccagtgtctacacagatgatgaagagagaggcaacgtcatgggaatcgccttgggaggcctggccatgggggtcttagtgggcccccccttcgggagtgtgctctatgagtttgtggggaagacggctccgttcctggtgctggccgccctggtactcttggatggagctattcagctctttgtgctccagccgtcccgggtgcagccagagagtcagaaggggacacccctaaccacgctgctgaaggacccgtacatcctcattgctgcaggctccatctgctttgcaaacatgggcatcgccatgctggagccagccctgcccatctggatgatggagaccatgtgttcccgaaagtggcagctgggcgttgccttcttgccagctagtatctcttatctcattggaaccaatatttttgggatacttgcacacaaaatggggaggtggctttgtgctcttctgggaatgataattgttggagtcagcattttatgtattccatttgcaaaaaacatttatggactcatagctccgaactttggagttggttttgcaattggaatggtggattcgtcaatgatgcctatcatgggctacctcgtagacctgcggcacgtgtccgtctatgggagtgtgtacgccattgcggatgtggcattttgtatggggtatgctataggtccttctgctggtggtgctattgcaaaggcaattggatttccatggctcatgacaattattgggataattgatattctttttgcccctctctgcttttttcttcgaagtccacctgccaaagaagaaaaaatggctattctcatggatcacaactgccctattaaaacaaaaatgtacactcagaataatatccagtcatatccgataggtgaagatgaagaatctgaaagtgactgagatgagatcctcaaaaatcatcaaagtgtttaattgtataaaacagtgtttccagtgacacaactcatccagaactgtcttagtcataccatccatccctggtgaaagagtaaaaccaaaggttattatttcctttccatggttatggtcgattgccaacagccttataaagaaaaagaagcttttctaggggtttgtataaatagtgttgaaactttattttatgtatttaattttattaaatatcatacaatatattttgatgaaataggtattgtgtaaatctataaatatttgaatccaaaccaaatataattttttaacttacattaacaaacatttgggcaaaaatcatattggtaatgagtgtttaaaattaaagcacacattatctctgagactcttccaacaaagagaaactagaatgaagtctgaaaaacagaatcaagtaagacagcatgttatatagtgacactgaatgttatttaacttgtagttactatcaatatatttatgcgttaaacagctagttctctcaagtgtagaggacaagaacttgtgtcagttatcttttgaatccataaatcttagctggcattagttttctatgtaatcacctacctagagagagttgtaaattatatgttaacatgttatctggttggcagcaaacactaaagccaataaaggaaaaacagtaaatgttccgaaagcagagaaaagcaaccaaacatattgttatgaactaaaagctttccctttaagatgcatacttgtcttactggatgaagaaaattgagggtacatgtaccttatactgtcaaggttgtttaaacatgataaggttaatcgccatctacttcaagttttagaaaaggaaacaagaagctgaaaacagctgctctgactttaatatctgactatatctttgatctgtttgcaggtcatccaagtgttttctaggaatatatttattttaggttgtctgaaactactattttttagactcctgaaagttgttcacatcaatgtgaagacaaattttaaatgaaaatgaagaatgaaattatgtcttgaatcatatattaagaagtaaaaataatagtgatcaggcagaaaagaaaaatggaacatctaaaaatgtatgtgctaactatatcatccagtgtgcagtgttgtgtatttttctaagcatgacaacattgatgtgccttttcagtgtaacagcaaatactgttagtgaacattgtcaatttatgtcattttgttaagagatatgactggagtgtgcagtgtggaatgtctctaatactacttgtgaatcctgcagttctataatcataaacaaaaattacttagtttcgttaagctaagattgtgtttgtgttaacttcgacatcaaggagcaaagaactttagaacagactcctcaatcttgtgactttcttattctctaggaaagtaacacttcgtttcatgaagcttttctgtggggcttcgattatttcaagtctggtttctaagtgcagtgtgtttgaagcaaacgaacttccaactcacttatttggcattgggcaacttggccaagtctgccactttggaagatggctctggaggaaactctcatatggctaaaaaggcaggctagtttcttacttctacaggggtagagccttaaaaaagaacgtgctacaaattggttctctttgagggtttctggttctccctgcccccaataccatatactttattgcaattttatttttgcctttacggctctgtgtctttctgcaagaaggcctggcaaaggtatgcctgctgttggtccctcgggataagataaaatataaataaaaccttcagaactgttttggagcaaaagatagcttgtacttggggaaaaaaattctaagttcttttatatgactaatattcttggttagcaagactggaaagaggtgtttttttaaaatgtacataccagaacaaagaacatacagctctctgaacatttattttttgaacagaggtggtttttatgtttggacctggtaatacagatacaaaaactttaatgaggtagcaatgaatattcaactgtttgactgctaagtgtatctgtccatattttagcaagtttacttaataaatcttctgaaccatgaaaaaaaaaaaaa

SEQ ID NO: 28 - DNA, 5486 bp, Homo sapiens
  source 1..5486: mol_type = other DNA
gccctcttctgaagaggcgtttctggaccactgagccccgcctcccactgtgagcggaaccctaccgtttttaaaaaaatctttttcaaaacttgccaggttgtctttccaaatatttttaataatagtgctgctgctgtagaccacagagaaaagaatccctcgctcttccttttcacttagtagaaacttctaccgcgtaggtcccgccaggagttcgcgcatgcgcaggagcgacaataagatggcggtgataatcgccgcactttttttcaaattagtggatcccagaaatcattgcgcgcatttgtaacgaatttccgttcgagtttgtattttaggcgccattttcgagtgaaggacccggagccgaaacaccggtaggagcggggaggtgggtactacacaaccgtctccagccttggtctgagtggactgtcctgcagcgaccatgccccgtaaaggcacccagccctccactgcccggcgcagagaggaagggccgccgccgccgtcccctgacggcgccagcagcgacgcggagcctgagccgccgtccggccgcacggagagcccagccaccgccgcagagactgcaagtgaggaacttgataatagaagtttagaagagattttgaacagcattcctcctcccccgcctccagcaatgaccaatgaagctggagctcctcggcttatgataactcatattgtaaaccagaacttcaaatcctatgctggggagaaaattctgggacctttccataagcgcttttcctgtattatcgggccaaatggcagtggcaaatccaatgttattgattctatgctttttgtgtttggctatcgagcacaaaaaataagatctaaaaaactctcagtattaatacataattctgatgaacacaaggacattcagagttgtacagtagaagttcattttcaaaagataattgataaggaaggggatgattatgaagtcattcctaacagtaatttctatgtatccagaacggcctgcagagataatacttctgtctatcacataagtggaaagaaaaagacatttaaggatgttggaaatcttcttcgaagccatggaattgacttggaccataatagatttttaattttacagggtgaagttgaacaaattgctatgatgaaaccaaaaggccagactgaacacgatgagggtatgcttgaatatttagaagatataattggttgtggacggctaaatgaacctattaaagtcttgtgtcggagagttgaaatattaaatgaacacagaggagagaagttaaacagggtaaagatggtggaaaaggaaaaggatgccttagaaggagagaaaaacatagctatcgaatttcttaccttggaaaatgaaatatttagaaaaaagaatcatgtttgtcaatattatatttatgagttgcagaaacgaattgctgaaatggaaactcaaaaggaaaaaattcatgaagataccaaagaaattaatgagaagagcaatatactatcaaatgaaatgaaagctaagaataaagatgtaaaagatacagaaaagaaactgaataaaattacaaaatttattgaggagaataaagaaaaatttacacagctagatttggaagatgttcaagttagagaaaagttaaaacatgccacgagtaaagccaaaaaactggagaaacaacttcaaaaagataaagaaaaggttgaagaatttaaaagtatacctgccaagagtaacaatatcattaatgaaacaacaaccagaaacaatgccctcgagaaggaaaaagagaaagaagaaaaaaaattaaaggaagttatggatagccttaaacaggaaacacaagggcttcagaaagaaaaagaaagtcgagagaaagaacttatgggtttcagcaaatcggtaaatgaagcacgttcaaagatggatgtagcccagtcagaacttgatatctatctcagtcgtcataatactgcagtgtctcaattaactaaggctaaggaagctctaattgcagcttctgagactctcaaagaaaggaaagctgcaatcagagatatagaaggaaaactccctcaaactgaacaagaattaaaggagaaagaaaaagaacttcaaaaacttacacaagaagaaacaaactttaaaagtttggttcatgatctctttcaaaaagttgaagaagcaaagagctcattagcaatgaatcgaagtagggggaaagtccttgatgcaataattcaagaaaaaaaatctggcaggattccaggaatatatggaagattgggggacttaggagccattgatgaaaaatacgacgtggctatatcatcctgttgtcatgcactggactacattgttgttgattctattgatatagcccaagaatgtgtaaacttccttaaaagacaaaatattggagttgcaacctttataggtttagataagatggctgtatgggcgaaaaagatgaccgaaattcaaactcctgaaaatactcctcgtttatttgatttagtaaaagtaaaagatgagaaaattcgccaagctttttattttgctttacgagataccttagtagctgacaacttggatcaagccacaagagtagcatatcaaaaagatagaagatggagagtggtaactttacagggacaaatcatagaacagtcaggtacaatgactggtggtggaagcaaagtaatgaaaggaagaatgggttcctcacttgttattgaaatctctgaagaagaggtaaacaaaatggaatcacagttgcaaaacgactctaaaaaagcaatgcaaatccaagaacagaaagtacaacttgaagaaagagtagttaagttacggcatagtgaacgagaaatgaggaacacactagaaaaatttactgcaagcatccagcgtttaatagagcaagaagaatatttgaatgtccaagttaaggaacttgaagctaatgtacttgctacagcccctgacaaaaaaaagcagaaattgctagaagaaaacgttagtgctttcaaaacagaatatgatgctgtggctgagaaagctggtaaagtagaagctgaggttaaacgcttacacaataccatcgtagaaatcaataatcataaactcaaggcccaacaagacaaacttgataaaataaataagcaattagatgaatgtgcttctgctattactaaagcccaagtagcaatcaagactgctgacagaaaccttcaaaaggcacaagactctgtcttgcgtacagagaaagaaataaaagatactgagaaagaggtggatgacctaacagcagagctgaaaagtcttgaggacaaagcagcagaggtcgtaaagaatacaaatgctgcagaggaatccttaccagagatccagaaagaacatcgcaatctgcttcaagaattaaaagttattcaagaaaatgaacatgctcttcaaaaagatgcacttagtattaagttgaaacttgaacaaatagatggtcacattgctgaacataattctaaaataaaatattggcacaaagagatttcaaaaatatcactgcatcctatagaagataatcctattgaagagatttcggttctaagcccagaggatcttgaagcgatcaagaatccagattctataacaaatcaaattgcacttttggaagcccggtgtcatgaaatgaaaccaaacctcggtgccatcgcagagtataaaaagaaggaagaattgtatttgcaacgggtagcagaattggacaaaattacttatgaaagagacagttttagacaggcatatgaagatcttcggaaacaaaggcttaatgaatttatggcaggtttttatataataacaaataaattaaaggaaaattaccaaatgcttactttgggaggggacgccgaactcgagcttgtagacagcttggatcctttctctgaaggaatcatgttcagtgttcgaccacctaagaaaagttggaaaaagatcttcaacctttcgggaggagagaaaacacttagttcattggctttagtatttgctcttcaccactacaagcccactcccctttacttcatggatgagattgatgcagcccttgattttaaaaatgtgtccattgttgcattttatatatatgaacaaacaaaaaatgcacagttcataataatttctcttcgaaataatatgtttgagatttcggatagacttattggaatttacaagacatacaacataacaaaaagtgttgctgtaaatccaaaagaaattgcatctaagggactttgttgaactttatgctgaagattcttcaagttgattcagtgtattactgatttttttctatttgtaaaggattatgagttgtataaaatacatactccctaaactagatcatgaaactggtttctgttttatgcagttgtcatttgtaaagtctaataaaatattctctataattgcttctagattacaaaaatatgacaatcttgtaagtagcagactatggagaaaaatgagttacctggagggtcaggtaacttgccaaactaaaaagtatgttagttgaggcaaagtcctaagcaaggttgtgctatcaaggctcagcataccttcgtgggcctttgatttaccaacactggaaatgcctgccaactaatcttggatagattctttaaggcattccacttagcttgccagttgagacaatcaccacagttattacccaaatactatgaacatatttttgtaaaccagtcattctgaattatagtgatgagaatttaaatatatgcttttctagaatttgatgtttgaccatttatgacttaattaccagagagccagtaaattaggacagtgtttcaacaagcctaggctatctcgtaagttgaaaaatatcccactatagttgcttcatgagtatgaagtaagatggcctctgatttacactggttcaatttacaaattttcaactttatgataggtttatccgggtactaaatgcatttcaacttgatagtttcaacttatgataggtttaccaggatgtagtcccactgttgaggagcatctatttaggggttaattactttagtaataagtggaaagtaagataccttgagtaatgtttgcctataaaattgtcagcgtatttttacactattggctcaagaatgttataatgctaagggacataagttggcaaccacttggtttttggaaggactttcggtattgtattagaagtctgccctagctgttaaatttctgggtatttatcctaaggaattaattaaagagttaattgttcctttcttcagtgggccattgttttagatatttaaaaaatccaacagtttctatcataatgtaactgtaaaaatgtaaacacattattagcatggacttttaaataaagatttaaagaaagcaagatcggaaaaaaaaaaaaaaaaaa

SEQ ID NO: 29 - DNA, 3601 bp, Homo sapiens
  source 1..3601: mol_type = other DNA
gggagaaggaggaggccgggggaaggaggagacaggaggaggagggaccacggggtggaggggagatagacccagcccagagctctgagtggtttcctgttgcctgtctctaaacccctccacattcccgcggtccttcagactgcccggagagcgcgctctgcctgccgcctgcctgcctgccactgagggttcccagcaccatgagggcctggatcttctttctcctttgcctggccgggagggccttggcagcccctcaagaagccctgcctgatgagacagaggtggtggaagaaactgtggcagaggtgactgaggtatctgtgggagctaatcctgtccaggtggaagtaggagaatttgatgatggtgcagaggaaaccgaagaggaggtggtggcggaaaatccctgccagaaccaccactgcaaacacggcaaggtgtgcgagctggatgagaacaacacccccatgtgcgtgtgccaggaccccaccagctgcccagcccccattggcgagtttgagaaggtgtgcagcaatgacaacaagaccttcgactcttcctgccacttctttgccacaaagtgcaccctggagggcaccaagaagggccacaagctccacctggactacatcgggccttgcaaatacatccccccttgcctggactctgagctgaccgaattccccctgcgcatgcgggactggctcaagaacgtcctggtcaccctgtatgagagggatgaggacaacaaccttctgactgagaagcagaagctgcgggtgaagaagatccatgagaatgagaagcgcctggaggcaggagaccaccccgtggagctgctggcccgggacttcgagaagaactataacatgtacatcttccctgtacactggcagttcggccagctggaccagcaccccattgacgggtacctctcccacaccgagctggctccactgcgtgctcccctcatccccatggagcattgcaccacccgctttttcgagacctgtgacctggacaatgacaagtacatcgccctggatgagtgggccggctgcttcggcatcaagcagaaggatatcgacaaggatcttgtgatctaaatccactccttccacagtaccggattctctctttaaccctccccttcgtgtttcccccaatgtttaaaatgtttggatggtttgttgttctgcctggagacaaggtgctaacatagatttaagtgaatacattaacggtgctaaaaatgaaaattctaacccaagacatgacattcttagctgtaacttaactattaaggccttttccacacgcattaatagtcccatttttctcttgccatttgtagctttgcccattgtcttattggcacatgggtggacacggatctgctgggctctgccttaaacacacattgcagcttcaacttttctctttagtgttctgtttgaaactaatacttaccgagtcagactttgtgttcatttcatttcagggtcttggctgcctgtgggcttccccaggtggcctggaggtgggcaaagggaagtaacagacacacgatgttgtcaaggatggttttgggactagaggctcagtggtgggagagatccctgcagaacccaccaaccagaacgtggtttgcctgaggctgtaactgagagaaagattctggggctgtgttatgaaaatatagacattctcacataagcccagttcatcaccatttcctcctttacctttcagtgcagtttcttttcacattaggctgttggttcaaacttttgggagcacggactgtcagttctctgggaagtggtcagcgcatcctgcagggcttctcctcctctgtcttttggagaaccagggctcttctcaggggctctagggactgccaggctgtttcagccaggaaggccaaaatcaagagtgagatgtagaaagttgtaaaatagaaaaagtggagttggtgaatcggttgttctttcctcacatttggatgattgtcataaggtttttagcatgttcctccttttcttcaccctccccttttttcttctattaatcaagagaaacttcaaagttaatgggatggtcggatctcacaggctgagaactcgttcacctccaagcatttcatgaaaaagctgcttcttattaatcatacaaactctcaccatgatgtgaagagtttcacaaatccttcaaaataaaaagtaatgacttagaaactgccttcctgggtgatttgcatgtgtcttagtcttagtcaccttattatcctgacacaaaaacacatgagcatacatgtctacacatgactacacaaatgcaaacctttgcaaacacattatgcttttgcacacacacacctgtacacacacaccggcatgtttatacacagggagtgtatggttcctgtaagcactaagttagctgttttcatttaatgacctgtggtttaacccttttgatcactaccaccattatcagcaccagactgagcagctatatccttttattaatcatggtcattcattcattcattcattcacaaaatatttatgatgtatttactctgcaccaggtcccatgccaagcactggggacacagttatggcaaagtagacaaagcatttgttcatttggagcttagagtccaggaggaatacattagataatgacacaatcaaatataaattgcaagatgtcacaggtgtgatgaagggagagtaggagagaccatgagtatgtgtaacaggaggacacagcattattctagtgctgtactgttccgtacggcagccactacccacatgtaactttttaagatttaaatttaaattagttaacattcaaaacgcagctccccaatcacactagcaacatttcaagtgcttgagagccatgcatgattagtggttaccctattgaataggtcagaagtagaatcttttcatcatcacagaaagttctattggacagtgctcttctagatcatcataagactacagagcacttttcaaagctcatgcatgttcatcatgttagtgtcgtattttgagctggggttttgagactccccttagagatagagaaacagacccaagaaatgtgctcaattgcaatgggccacatacctagatctccagatgtcatttcccctctcttattttaagttatgttaagattactaaaacaataaaagctcctaaaaaatcaaactgtattctggtgttctcttctacacagtgggagggcgagcagtaggagagattggcccatttggtgctggccatttgaggaatgcaagcccagcactagtctcataatctctaggaatctgtagagagaggaattgaagtaaatttcagcattggctcattcagtcattcggcgacattcatcaggtacctgcaatgtgttaggggatcttatgagtaggcagcgtgcgtgatccttgctcccctggagctttctaacattctagcaggcagaccacacataaatttgcaatactgtttctgataaaaacgtgctgtaaaggaaataaagcagagaactatcatggaaaaaaaaaaaaaaaaaa

SEQ ID NO: 30 - DNA, 2989 bp, Homo sapiens
  source 1..2989: mol_type = other DNA
gtctgggccgagcccgcccagctggctgagacgcgtggagcctggcggcgagtgggggcgtgcgacggttactctggttactggggccgcgccgcgctggcgagagccgccgcccgcgagggatgctggtgaggaagccgtcgggagccgccgccgccatctgagggaggtaccctggaaaccaccttttatcggtggggaagtgcagtcgcggtgggcggctctgggggccagcgaaacgggaggcctctaaatctttaggttggggctgcattgccctggagccgcactcttgagtccgaggccatcttttgttggagaaggcgtcggcgttggcgttttcccgaggttgggctgtacagtgtctccgtccgcggaaaaagaagcctctgaacccgcgccggcccgcagcccccgtgccttccggccgctgctcgccgtcgccagaggctaggccacgtttcccccagtgccgaggtgtttctgtgaccctccctccactcccattcccttctgaaagggcacctgctcttggtgagaaaagaaattatagcacgaagagccagtatcagaagagtatccatcacccgcagcaaccgctcagggaacaccatcaaaaaagaaaaaaagggaatatctggatttcctgggcgaggaggagcgagtctgctcgggagctgttccagcaggcgatttttaaatactgctttctacgccctatacaacttggcttcacatacttttacactaactttatatgatttttaaaaactggtctgatcggacttctcgtcctgggacactgtttactggagtctggccggctctccgtgctcctcttggtacctcattttggggagaaccttaaacccactcgagcagataatctccgccttgaccggtgccaccaaagaagccttggaaccatgtggacttttctgggcattgccactttcacctatttttataagaagttcggggacttcatcactttggccaacagggaggtcctgttgtgcgtgctggtgttcctctcgctgggcctggtgctctcctaccgctgtcgccaccgaaacgggggtctcctcgggcgccagcagagcggctcccagttcgccctcttctcggatattctctcaggcctgcctttcattggcttcttctgggccaaatccccccctgaatcagaaaataaggagcagctcgaggccaggaggcgcagaaaaggaaccaatatttcagaaacaagcttaataggaacagctgcctgtacatcaacatcttctcagaatgacccagaagttatcatcgtgggagctggcgtgcttggctctgctttggcagctgtgctttccagagatggaagaaaggtgacagtcattgagagagacttaaaagagcctgacagaatagttggagaattcctgcagccgggtggttatcatgttctcaaagaccttggtcttggagatacagtggaaggtcttgatgcccaggttgtaaatggttacatgattcatgatcaggaaagcaaatcagaggttcagattccttaccctctgtcagaaaacaatcaagtgcagagtggaagagctttccatcacggaagattcatcatgagtctccggaaagcagctatggcagagcccaatgcaaagtttattgaaggtgttgtgttacagttattagaggaagatgatgttgtgatgggagttcagtacaaggataaagagactggagatatcaaggaactccatgctccactgactgttgttgcagatgggcttttctccaagttcaggaaaagcctggtctccaataaagtttctgtatcatctcattttgttggctttcttatgaagaatgcaccacagtttaaagcaaatcatgctgaacttattttagctaacccgagtccagttctcatctaccagatttcatccagtgaaactcgagtacttgttgacattagaggagaaatgccaaggaatttaagagaatacatggttgaaaaaatttacccacaaatacctgatcacctgaaagaaccattcttagaagccactgacaattctcatctgaggtccatgccagcaagcttccttcctccttcatcagtgaagaaacgaggtgttcttcttttgggagacgcatataatatgaggcatccacttactggtggaggaatgactgttgcttttaaagatataaaactatggagaaaactgctaaagggtatccctgacctttatgatgatgcagctattttcgaggccaaaaaatcattttactgggcaagaaaaacatctcattcctttgtcgtgaatatccttgctcaggctctttatgaattattttctgccacagatgattccctgcatcaactaagaaaagcctgttttctttatttcaaacttggtggcgaatgtgttgcgggtcctgttgggctgctttctgtattgtctcctaaccctctagttttaattggacacttctttgctgttgcaatctatgccgtgtatttttgctttaagtcagaaccttggattacaaaacctcgagcccttctcagtagtggtgctgtattgtacaaagcgtgttctgtaatatttcctctaatttactcagaaatgaagtatatggttcattaagcttaaaggggaaccatttgtgaatgaatatttggaacttaccaagtcctaagagacttttggaagaggatatatatagcatagtaccataccacttataaagtggaaactcttggaccaagatttggattaatttgtttttgaagttttttgtatataaatatgtaaatacatgctttaatttgcaatttaaaatgaaggggttaaataagttagacatttaaaagaaatgattgttaccataaattagtgctaatgctgaggagaactacagtttttcttttgaatttagtatttgagatgagttgttgggacatgcaaataaaatgaagaatgaa

SEQ ID NO: 31 - DNA, 3138 bp, Homo sapiens
  source 1..3138: mol_type = other DNA
attttacccagcccctgttcaagatggagttgctgtggttcacacatctctgacaaaaatacagggctattcggagtcaccagacctggagtttgagtatgctgacacagacaagtgggctgcagagctctcggagctttacagctacacggaagggccagaattcctgatgaatcgaaaatgctttgaggaggacttccggatccatgtgacagacaagaagtggactgagctggataccaaccagcaccggacccatgccatgaggctcctggatggcttggaagtcactgccagggagaagagactcaaggtggctcgagcaattctctatgttgctcaaggcacgtttggggagtgcagctcggaggcagaggtgcagtcctggatgcgctacaacatctttctcctcctggaggtgggcacgttcaatgctttggtggagcttctgaacatggaaatagacaacagtgccgcctgcagcagtgctgtgaggaagcctgccatctccctggctgacagcacagacctcagggtcctgctcaacatcatgtacctgatagtggagaccgttcatcaggagtgtgagggtgacaaggctgagtggaggaccatgcggcagaccttcagagccgagctgggctccccgctgtacaacaatgagccatttgccatcatgctgtttgggatggtgaccaaattttgcagtggtcacgcccctcactttcccatgaagaaagttctcttgctgctctggaagacagtattgtgcacgctaggcggctttgaggagctgcagagcatgaaggctgagaagcgcagcatcctgggcctccccccgcttcctgaggacagcatcaaagtgattcgcaacatgagagcagcctctccaccagcatctgcttcagacttgattgagcagcagcagaaacggggccgccgagagcacaaggctctgataaagcaggacaacctagatgccttcaacgagcgggatccctacaaggctgatgactctcgagaagaggaagaggagaatgatgatgacaacagtctggagggggagacgtttcccctggaacgggatgaagtgatgcctcccccgctacagcacccacagactgacaggctgacttgccccaaagggctcccgtgggctcccaaggtcagagagaaagacattgagatgttccttgagtccagccgcagcaaatttataggttacactctaggcagtgacacgaacacagtggtggggctgcccaggccaatccacgaaagcatcaagactctgaaacagcacaagtacacgtcgattgcagaggtccaggcacagatggaggaggaatacctccgctcccctctctcagggggagaagaagaagttgagcaagtccctgcagaaaccctctaccaaggcttgctccccagcctgcctcagtatatgattgccctcctgaagatcctgttggctgcagcacccacctcaaaagccaaaacagactcaatcaacatcctagcggacgtcttgcctgaggagatgcccaccacagtgttgcagagcatgaagctgggggtggatgtaaaccgccacaaagaggtcattgttaaggccatttctgctgtcctgctgctgctgctcaagcactttaagttgaaccatgtctaccagtttgaatacatggcccagcacctggtgtttgccaactgcattcctttgatcctaaagttcttcaatcaaaacatcatgtcctacatcactgccaagaacagcatttctgtcctggattaccctcactgcgtggtgcatgagctgccagagctgacggcggagagtttggaagcaggtgacagtaaccaattttgctggaggaacctcttttcttgtatcaatctgcttcggatcttgaacaagctgacaaagtggaagcattcaaggacaatgatgctggtggtgttcaagtcagcccccatcttgaagcgggccctaaaggtgaaacaagccatgatgcagctctatgtgctgaagctgctcaaggtacagaccaaatacttggggcggcagtggcgaaagagcaacatgaagaccatgtctgccatctaccagaaggtgcggcatcggctgaacgacgactgggcatacggcaatgatcttgatgcccggccttgggacttccaggcagaggagtgtgcccttcgtgccaacattgaacgcttcaacgcccggcgctatgaccgggcccacagcaaccctgacttcctgccagtggacaactgcctgcagagtgtcctgggccaacgggtggacctccctgaggactttcagatgaactatgacctctggttagaaagggaggtcttctccaagcccatttcctgggaagagctgctgcagtgaggctgttggttaggggactgaaatggagagaaaagatgatctgaaggtacctgtgggactgtcctagttcattgctgcagtgctcccatcccccaccaggtggcagcacagccccactgtgtcttccgcagtctgtcctgggcttgggtgagcccagcttgacctccccttggttcccagggtcctgctccgaagcagtcatctctgcctgagatccattcttcctttacttcccccaccctcctctcttggatatggttggttttggctcatttcacaatcagcccaaggctgggaaagctggaatgggatgggaacccctccgccgtgcatctgaatttcaggggtcatgctgatgcctctcgagacatacaaatccttgctttgtcagcttgcaaaggaggagagtttaggattagggccagggccagaaagtcggtatcttggttgtgctctggggtgggggtggggtgtttctgatgttattccagcctcctgctacattatatccagaagtaattgcggaggctccttcagctgcctcagcactttgattttggacagggacaaggtaggaagagaagcttcccttaaccagaggggccatttttccttttggctttcgagggcctgtaaatatctatatataattctgtgtgtattctgtgtcatgttggggtttttaatgtgattgtgtattctgtttacattaaaaagaagcaaaaataaaaaaaaaaaaa

SEQ ID NO: 32 - DNA, 3098 bp, Homo sapiens
  source 1..3098: mol_type = other DNA
gcacgcagccttagctccttccccgcccttgctcttcccagtttctccgtcagcctgcgggtcccggctggcggctgcttccggtaggagagcggtgtagagcgagcaggtctcagctcctcgtcatgtcatacggtcccttagacatgtaccggaacccggggccctcggggccccagctccgggacttcagcagcatcatccagacgtgcagcggcaacatccagcggatcagccaagccactgctcagataaagaatttgatgagccagctaggaactaagcaggactcaagcaagctacaggaaaatctgcaacagttacaacactccacaaatcagctcgccaaggaaacaaatgaattgctgaaagaattagggtccttgccccttcccttatctacttcagaacagcgccagcagagacttcagaaggaacgcctcatgaatgacttctctgcagccttaaacaatttccaggctgtgcagagaagggtatctgaaaaggaaaaggagagtattgccagagcaagagctggatctcgtctttctgcagaagagaggcaaagagaggagcagctggtctcatttgacagccatgaggagtggaaccagatgcagagccaggaggatgaggtggccatcactgagcaggatttggaacttattaaagaaagagaaacggcaattcggcagctggaggctgacattttggatgtcaatcagatatttaaagatttggccatgatgatccatgaccagggtgatctgattgatagcatagaagccaatgtggaaagctcagaggtgcacgtcgaaagagccactgaacagttacagcgagctgcttactatcagaaaaaatctcgcaagaagatgtgtatcctggtgcttgtcctgtcagtgattattctaatcttgggacttattatctggctagtttataaaacgaagtgattgcctccgatcgttctcccgctgagctgttttcaagggcaagtgcttgttgaagtcttgccagaacaaactgatcacaagaagacagcatatatcagaacgtcctgtaatcatttagttagaaactaactactaactagtctttggaattcgtgacctatggagacagtaattatcaatttattgattctattgatttctcaaattaggaattaacttatgtggattttgcttcctcttgtattctgattgcccttcatcccaagtgtttactgaaaattccattctagatattcttgttttgacaaatgacactacagtctcgtaatattgtcttttatgtatatacaaaatttacctttttactagcatctgagatagagttactttctggtacccagtatattggagtctgtcagaaactctataataggccaccagtttttattatttaacatttttatttgaatttctaagaagcctattctctatctattttgaaagattttggcactatatttaattggaaggtaaaatattgtacatgtgatccagagtaaatgagaagtctctatctgagctggtcagttactggagtacatgttactaatctgggtttaaagtttacttcattatctgctagtgtcatccacagcagttcatcctcatccacactaagccatcctgttagcttttaaaggaagttaatttaattaacattaatatactctatgggctccctctcccacctgtctgcatagaaaggcagaattagacatagcatgctttggaaaagcaaataggaattgttgggaatgatttaatcttgttgttgttgttgttgttgttcacttgtggttctacattcctggtgaatgatgaatgttgctgtcaaagggctgccccctaccttataagggttgctgggcatttgaaggcaggaagatttttaaagatagattgaggttggtttaaaattattcctgtaaaccaacaataaagcaaagaagaggttcatttttgtaaataacactggtttcaaatagtgatgttagacttaacctaatttataaacaagagattaatatctccatgcatagttttagacaaaaaaagatgtttcaataaaattactgtcttgtaatataaatgttgtccacttcccttttccacaggcctagaacagttaaagggaacataatttgtttaggctcccacataaatgtgaatctggccaacaactttggttcatcctttagtgaattagaggatttggctaccctgagtatatttatattcatttcttctgttctccttctgttattatacttaatcttctaaactaaactaatgtgaacagtagggaagcaagggcccaaatgcataagtttctttgcactgttgcacttacttaatacaaataaatgttttttaaagcttttgtagtatgtttttatgagttaacatcctaatgtggtaggtattaggtaatgtgctgtcatgagaaaaattgagacttccaagaaaactggacaccaggtgagggttggtttggagacggaataggtgtagctgcctttccttgaaaaacagtgtgtagagatggctgagtgcaatggctcacacttgtaatcccaacactttggaaggctgaggcgggaggatcagtatatcggtacgtctgagcccaagagttcaagatcagcctgggcagtatagcaagaccccatctccattttttttttaatgattttttaattaaaaaaaagaacaacaggatagagctgttggggtggcacagtggcccaaagagcagcttcagagataattccttggttctatgatccctgtttaactccaaattacagtcggacttggatacatcatttgtaacattgtaggaaagaaaaaagtcttggttgtgaaaaacgatttgcatttgggtaaaataaagtgaccatgcttttgttctgtaatactgtgtgacctgtggttgttgtaatggtgatcatggagagcaaatatgaacttggcctggattttaaatggcctagaatttgtggtagttgccaaagaggttctcctaggtggtcttaataaacctattcacagaattctccttaaaaaaaaaaaaaaaaaaaa

SEQ ID NO: 33 - DNA, 293 bp, Homo sapiens
  source 1..293: mol_type = other DNA
taggcgcgagctaagcaggaggcggaggcggaggcggagggcgaggggcggggagcgccgcctggagcgcggcaggaagccttatcagttgtgagtgaggaccagtcgttgtttgagtgtgcctacggaacgccacacctggctaagacagagatgaccgcgtcctcctccagcgactatggacagacttccaagatgagcccacgcgtccctcagcaggattggctgtctcaacccccagccagggtcaccatcaaaatggaatgtaaccctagccaggtgaatggctcaag

SEQ ID NO: 34 - DNA, 3112 bp, Homo sapiens
  source 1..3112: mol_type = other DNA
gggaacaaaagctggagctccaccgcggtggcggccgctctagccctcaaggaactctcctgatgaatgcagtgtggccaaaggcgggaagatggtgggcagcccagacaccgttgggatgaactacggcagctacatggaggagaagcacatgccacccccaaacatgaccacgaacgagcgcagagttatcgtgccagcagatcctacgctatggagtacagaccatgtgcggcagtggctggagtgggcggtgaaagaatatggccttccagacgtcaacatcttgttattccagaacatcgatgggaaggaactgtgcaagatgaccaaggacgacttccagaggctcacccccagctacaacgccgacatccttctctcacatctccactacctcagagagactcctcttccacatttgacttcagatgatgttgataaagccttacaaaactctccacggttaatgcatgctagaaacacagggggtgcagcttttattttcccaaatacttcagtatatcctgaagctacgcaaagaattacaactaggccagatttaccatatgagccccccaggagatcagcctggaccggtcacggccaccccacgccccagtcgaaagctgctcaaccatctccttccacagtgcccaaaactgaagaccagcgtcctcagttagaaccttatcagattcttggaccaacaagtagccgccttgcaaatccaggcagtggccagatccagctttggcagttcctcctggagctcctgtcggacagctccaactccagctgcatcacctgggaaggcaccaacggggagttcaagatgacggatcccgacgaggtggcccggcgctggggagagcggaagagcaaacccaacatgaactacgataagctcagccgcgccctccgttactactatgacaagaacatcatgaccaaggtccatgggaagcgctacgcctacaagttcgacttccacgggatcgcccaggccctccagccccaccccccggagtcatctctgtacaagtacccctcagacctcccgtacatgggctcctatcacgcccacccacagaagatgaactttgtggcgccccaccctccagccctccccgtgacatcttccagtttttttgctgccccaaacccatactggaattcaccaactgggggtatataccccaacactaggctccccaccagccatatgccttctcatctgggcacttactactaaagacctggcggaggcttttcccatcagcgtgcattcaccagcccatcgccacaaactctatcggagaacatgaatcaaaagtgcctcaagaggaatgaaaaaagctttactggggctggggaaggaagccggggaagagatccaaagactcttgggagggagttactgaagtcttactgaagtcttactacagaaatgaggaggatgctaaaaatgtcacgaatatggacatatcatctgtggactgaccttgtaaaagacagtgtatgtagaagcatgaagtcttaaggacaaagtgccaaagaaagtggtcttaagaaatgtataaactttagagtagagtttgaatcccactaatgcaaactgggatgaaactaaagcaatagaaacaacacagttttgacctaacataccgtttataatgccattttaaggaaaactacctgtatttaaaaatagaaacatatcaaaaacaagagaaaagacacgagagagactgtggcccatcaacagacgttgatatgcaactgcatggcatgtgctgttttggttgaaatcaaatacattccgtttgatggacagctgtcagctttctcaaactgtgaagatgacccaaagtttccaactcctttacagtattaccgggactatgaactaaaaggtgggactgaggatgtgtatagagtgagcgtgtgattgtagacagaggggtgaagaaggaggaggaagaggcagagaaggaggagaccagggctgggaaagaaacttctcaagcaatgaagactggactcaggacatttggggactgtgtacaatgagttatggagactcgagggttcatgcagtcagtgttataccaaacccagtgttaggagaaaggacacagcgtaatggagaaaggggaagtagtagaattcagaaacaaaaatgcgcatctctttctttgtttgtcaaatgaaaattttaactggaattgtctgatatttaagagaaacattcaggacctcatcattatgtgggggctttgttctccacagggtcaggtaagagatggccttcttggctgccacaatcagaaatcacgcaggcattttgggtaggcggcctccagttttcctttgagtcgcgaacgctgtgcgtttgtcagaatgaagtatacaagtcaatgtttttccccctttttatataataattatataacttatgcatttatacactacgagttgatctcggccagccaaagacacacgacaaaagagacaatcgatataatgtggccttgaattttaactctgtatgcttaatgtttacaatatgaagttattagttcttagaatgcagaatgtatgtaataaaataagcttggcctagcatggcaaatcagatttatacaggagtctgcatttgcactttttttagtgactaaagttgcttaatgaaaacatgtgctgaatgttgtggattttgtgttataatttactttgtccaggaacttgtgcaagggagagccaaggaaataggatgtttggcacccaaatggcgtcagcctctccaggtccttcttgcctcccctcctgtcttttatttctagccccttttggaacagaaggaccccgggtttcacattggagcctccatatttatgcctggaatggaaagaggcctatgaagctggggttgtcattgagaaattctagttcagcacctggtcacaaatcacccttaattcctgctatgattaaaatacatttgttgaacagtgaacaagctaccactcgtaaggcaaactgtattattactggcgggcggatcccccgggctgcaggaattcgatatcaagcttatcgataccgtcga

SEQ ID NO: 35 - DNA, 3037 bp, Homo sapiens
  source 1..3037: mol_type = other DNA
ctcctcacaggtgtgtctctagtcctcgtggttgcctgccccactccctgccgagacgcctgccagaaaggtcacctatcctgaaccccagcaagcctgaaacagctcagccaagcaccctgcgatggaagctgcagatgcctccaggagcaacgggtcgagcccagaagccagggatgcccggagcccgtcgggccccagtggcagcctggagaatggcaccaaggctgacggcaaggatgccaagaccaccaacgggcacggcggggaggcagctgagggcaagagcctgggcagcgccctgaagccaggggaaggtaggagcgccctgttcgcgggcaatgagtggcggcgacccatcatccagtttgtcgagtccggggacgacaagaactccaactacttcagcatggactctatggaaggcaagaggtcgccgtacgcagggctccagctgggggctgccaagaagccacccgttacctttgccgaaaagggcgagctgcgcaagtccattttctcggagtcccggaagcccacggtgtccatcatggagcccggggagacccggcggaacagctacccccgggccgacacgggccttttttcacggtccaagtccggctccgaggaggtgctgtgcgactcctgcatcggcaacaagcagaaggcggtcaagtcctgcctggtgtgccaggcctccttctgcgagctgcatctcaagccccacctggagggcgccgccttccgagaccaccagctgctcgagcccatccgggactttgaggcccgcaagtgtcccgtgcatggcaagacgatggagctcttctgccagaccgaccagacctgcatctgctacctttgcatgttccaggagcacaagaatcatagcaccgtgacagtggaggaggccaaggccgagaaggagacggagctgtcattgcaaaaggagcagctgcagctcaagatcattgagattgaggatgaagctgagaagtggcagaaggagaaggaccgcatcaagagcttcaccaccaatgagaaggccatcctggagcagaacttccgggacctggtgcgggacctggagaagcaaaaggaggaagtgagggctgcgctggagcagcgggagcaggatgctgtggaccaagtgaaggtgatcatggatgctctggatgagagagccaaggtgctgcatgaggacaagcagacccgggagcagctgcatagcatcagcgactctgtgttgtttctgcaggaatttggtgcattgatgagcaattactctctccccccacccctgcccacctatcatgtcctgctggagggggagggcctgggacagtcactaggcaacttcaaggacgacctgctcaatgtatgcatgcgccacgttgagaagatgtgcaaggcggacctgagccgtaacttcattgagaggaaccacatggagaacggtggtgaccatcgctatgtgaacaactacacgaacagcttcgggggtgagtggagtgcaccggacaccatgaagagatactccatgtacctgacacccaaaggtggggtccggacatcataccagccctcgtctcctggccgcttcaccaaggagaccacccagaagaatttcaacaatctctatggcaccaaaggtaactacacctcccgggtctgggagtactcctccagcattcagaactctgacaatgacctgcccgtcgtccaaggcagctcctccttctccctgaaaggctatccctccctcatgcggagccaaagccccaaggcccagccccagacttggaaatctggcaagcagactatgctgtctcactaccggccattctacgtcaacaaaggcaacgggattgggtccaacgaagccccatgagctcctggcggaaggaacgaggcgccacacccctgctcttcctcctgaccctgctgctcttgccttctaagctactgtgcttgtctgggtgggagggagcctggtcctgcacctgccctctgcagccctctgccagcctcttgggggcagttccggcctctccgacttccccactggccacactccattcagactcctttcctgccttgtgacctcagatggtcaccatcattcctgtgctcagaggccaacccatcacaggggtgagataggttggggcctgccctaacccgccagcctcctcctctcgggctggatctgggggctagcagtgagtacccgcatggtatcagcctgcctctcccgcccacgccctgctgtctccaggcctatagacgtttctctccaaggccctatcccccaatgttgtcagcagatgcctggacagcacagccacccatctcccattcacatggcccacctcctgcttcccagaggactggccctacgtgctctctctcgtcctacctatcaatgcccagcatggcagaacctgcagcccttggccactgcagatggaaacctctcagtgtcttgacatcaccctacccaggcggtgggtctccaccacagccactttgagtctgtggtccctggagggtggcttctcctgactggcaggatgaccttagccaagatattcctctgttccctctgctgagataaagaattcccttaacatgatataatccacccatgcaaatagctactggcccagctaccatttaccatttgcctacagaatttcattcagtctacactttggcattctctctggcgatggagtgtggctgggctgaccgcaaaaggtgccttacacactgcccccaccctcagccgttgccccatcagaggctgcctcctccttctgattaccccccatgttgcatatcagggtgctcaaggattggagaggagacaaaaccaggagcagcacagtggggacatctcccgtctcaacagccccaggcctatgggggctctggaaggatgggccagcttgcaggggttggggagggagacatccagcttgggctttcccctttggaataaaccattggtctgtcaaaaaaaaaaaaaaaaaaa

SEQ ID NO: 36 - DNA, 4035 bp, Homo sapiens
  source 1..4035: mol_type = other DNA
acttaacaaccgaagtaacccgcaatgcggaagggcgaggggattgcgagtcaccgagtttcccgcgcggcttgagtcacggcctagaaagagagatgttggggttcccaggaccaggacagaggtggtagtgaactctcatgggcatccagagaaggtcaggccccttgctgacaggcctatctgtggggctactgctgctcttcagctgggtgacccttgtccagccaacctctctctcagctctggtccaccaccctcacttgtgccagaccacccgggatgtccatggccgtcactaccctggtttcttttgccctcgtctgtctgattctccagaggaagcctactgctgccacctgcaggctgcagggggctcctgctgcacccgggctgaatttgaggccctgtaccaagtcaatctgtccgctcttccgcccccgcccatcctcaggggcccaggcccgctcctagtgctgggcctctacaacctactggttgtgaccctgatgaccgtagacctcgtgcacttctgctgcggtcggggccggagtctgggctggagccaccgcaggcctccctctgggtcctccgccgcgagctccctgcaggtctctgcggggacagcttaggtgcgcccggagcttgcctgcacctgcgatccagagccaagcgccccgcccctgcccgggcgcgctccctccttagccctgcccctctctgaccccacctccgacgcaagagtggggcggggcagctgccggtggcgtcccgaacccagactcgccccgccccagagactgcgcctgcgcgggcacgagacaacctctccgcgatgactgccagctcagtggagcagctgcggaaggagggcaatgagctgttcaaatgtggagactacgggggcgccctggcggcctacactcaggccctgggtctggacgcgacgccccaggaccaggccgttctgcaccggaaccgggccgcctgccacctcaagctggaagattacgacaaagcagaaacagaggcatccaaagccattgaaaaggatggtggggatgtcaaagcactctaccggcggagccaagccctagagaagctgggccgcctggaccaggctgtccttgacctgcagagatgtgtgagcttggagcccaagaacaaagttttccaggaggccttgcggaacatcgggggccagattcaggagaaggtgcgatacatgtcctcgacggatgccaaagtggaacagatgtttcagatactgttggacccagaagagaagggcactgagaaaaagcaaaaggcttctcagaacctggtggtgctggccagggaggatgctggagcggagaagatcttccggagtaatggggttcagctcttgcaacgtttactggacatgggagagactgacctcatgctggcggctctgcgtacgctggttggcatttgctctgagcatcagtcacggacagtggcaaccctgagcatactgggaactcggcgagtagtctccatcctgggcgtggaaagccaggctgtgtccctggctgcctgccacctgctgcaggttatgtttgatgccctcaaggaaggtgtcaaaaaaggcttccgaggcaaagaaggtgccatcattgtggatcctgcccgggagctgaaggtcctcatcagtaacctcttagatctgctgacagaggtgggggtctctggccaaggccgagacaatgccctgaccctcctgattaaagcggtgccccggaagtctctcaaggaccccaacaacagcctcaccctctgggtcatcgaccaaggtctgaaaaagattttggaagtggggggctctctacaggaccctcctggggagctcgcagtgaccgcaaacagccgcatgagcgcctctattctcctcagcaagctctttgatgacctcaagtgtgatgcggagagggagaatttccacagactttgtgaaaactacatcaagagctggtttgagggccaagggctggccgggaagctacgggccatccagacggtgtcctgcctcctgcagggcccatgtgacgctggcaaccgggccttggagctgagcggtgtcatggagagtgtgattgctctgtgtgcctctgagcaggaggaggagcagctggtggccgtggaggctctgatccatgcagccggcaaggctaagcgggcctcattcatcactgccaatggtgtctcgctgctgaaggacctatataagtgcagcgagaaggacagcatccgcatccgggcgctagtgggactctgtaagctcggttcggctggagggactgacttcagcatgaagcagtttgctgaaggctccactctcaaactggctaagcagtgtcgaaagtggctgtgcaatgaccagatcgacgcaggcactcggcgctgggcagtggagggcctggcttacctgacctttgatgccgacgtgaaggaagagtttgtggaggatgcggctgctctgaaagctctgttccagctcagcaggttggaggagaggtcagtgctctttgcggtggcctcagcgctggtgaactgcaccaacagctatgactacgaggagcccgaccccaagatggtggagctggccaagtatgccaagcagcatgtgcccgagcagcaccccaaggacaagccaagcttcgtgcgggctcgggtgaagaagctgctggcagcgggtgtggtgtcggccatggtgtgcatggtgaagacggagagccctgtgctgaccagttcctgcagagagctgctctccagggtcttcttggctttagtggaagaggtagaggaccgaggcactgtggttgcccagggaggcggcagggcgctgatcccgctggccctggaaggcacggacgtggggcagacaaaggcagcccaggcccttgccaagctcaccatcacctccaacccggagatgaccttccctggcgagcggatctatgaggtggtccggcccctcgtctccctgttgcacctcaactgctcaggcctgcagaacttcgaggcgctcatggccctaacaaacctggctgggatcagcgagaggctccggcagaagatcctgaaggagaaggctgtgcccatgatagaaggctacatgtttgaggagcatgagatgatccgccgggcagccacggagtgcatgtgtaacttggccatgagcaaggaggtgcaggacctcttcgaagcccagggcaatgaccgactgaagctgctggtgctgtacagtggagaggatgatgagctgctacagcgggcagctgccgggggcttggccatgcttacctccatgcggcccacgctctgcagccgcattccccaagtgaccacacactggctggagatcctgcaggccctgcttctgagctccaaccaggagctgcagcaccggggtgctgtggtggtgctgaacatggtggaggcctcgagggagattgccagcaccctgatggagagtgagatgatggagatcttgtcagtgctagctaagggtgaccacagccctgtcacaagggctgctgcagcctgcctggacaaagcagtggaatatgggcttatccaacccaaccaagatggagagtgagggggttgtccctgggcccaaggctcatgcacacgctacctattgtggcacggagagtaaggacggaagcagctttggctggtggtggctggcatgcccaatactcttgcccatcctcgcttgctgccctaggatgtcctctgttctgagtcagcggccacgttcagtcacacagccctgcttggccagcactgcctgcagcctcactcagaggggccctttttctgtactactgtagtcagctgggaatggggaaggtgcatcccaacacagcctgtggatcctggggcatctggaagggcgcacacatcagcagcctcaccagctgtgagcctgctatcaggcctgcccctccaataaaagtgtgtagaactccaaaaaaaaaaaaaaaaaaaaaaaaaaaaaaaaaa

SEQ ID NO: 37 - DNA, 3729 bp, Homo sapiens
  source 1..3729: mol_type = other DNA
cgaaggggcgtggccaagcgcaccgcctcggggcggggccggcgttctagcgcatcgcggccgggtgcgtcactcgcgaagtggaatttgcccagacaagcaacatggctcggaaacgcgcggccggcggggagccgcggggacgcgaactgcgcagccagaaatccaaggccaagagcaaggcccggcgtgaggaggaggaggaggatgcctttgaagatgagaaacccccaaagaagagccttctctccaaagtttcacaaggaaagaggaaaagaggctgcagtcatcctgggggttcagcagatggtccagcaaaaaagaaagtggccaaggtgactgttaaatctgaaaacctcaaggttataaaggatgaagccctcagcgatggggatgacctcagggactttccaagtgacctcaagaaggcacaccatctgaagagaggggctaccatgaatgaagacagcaatgaagaagaggaagaaagtgaaaatgattgggaagaggttgaagaacttagtgagcctgtgctgggtgacgtgagagaaagtacagccttctctcgatctcttctgcctgtgaagccagtggagatagagattgaaacgccagagcaggcgaagacaagagaaagaagtgaaaagataaaactggagtttgagacatatcttcggagggcgatgaaacgtttcaataaaggggtccatgaggacacacacaaggttcaccttctctgcctgctagcaaatggcttctatcgaaataacatctgcagccagccagatctgcatgctattggcctgtccatcatcccagcccgctttaccagagtgctgcctcgagatgtggacacctactacctctcaaacctggtgaagtggttcattggaacatttacagttaatgcagaactttcagccagtgaacaagataacctgcagactacattggaaaggagatttgctatttactctgctcgagatgatgaggaattggtccatatattcttactgattctccgggctctgcagctcttgacccggctggtattgtctctacagccaattcctctgaagtcagcaacagcaaagggaaagaaaccttccaaggaaagattgactgcggatccaggaggctcctcagaaacttccagccaagttctagaaaaccacaccaaaccaaagaccagcaaaggaaccaaacaagaggaaacctttgctaagggcacctgcaggccaagtgccaaagggaagaggaacaagggaggcagaaagaaacggagcaagccctcctccagcgaggaagatgagggcccaggagacaagcaggagaaggcaacccagcgacgtccgcatggccgggagcggcgggtggcctccagggtgtcttataaagaggagagtgggagtgatgaggctggcagcggctctgattttgagctctccagtggagaagcctctgatccctctgatgaggattccgaacctggccctccaaagcagaggaaagcccccgctcctcagaggacaaaggctgggtccaagagtgcctccaggacccatcgtgggagccatcgtaaggacccaagcttgccagcggcatcctcaagctcttcaagcagtaaaagaggcaagaaaatgtgcagcgatggtgagaaggcagaaaaaagaagcatagctggtatagaccagtggctagaggtgttctgtgagcaggaggaaaagtgggtatgtgtagactgtgtgcacggtgtggtgggccagcctctgacctgttacaagtacgccaccaagcccatgacctatgtggtgggcattgacagtgacggctgggtccgagatgtcacacagaggtacgacccagtctggatgacagtgacccgcaagtgccgggttgatgctgagtggtgggccgagaccttgagaccataccagagcccatttatggacagggagaagaaagaagacttggagtttcaggcaaaacacatggaccagcctttgcccactgccattggcttatataagaaccaccctctgtatgccctgaagcggcatctcctgaaatatgaggccatctatcccgagacagctgccatccttgggtattgtcgtggagaagcggtctactccagggattgtgtgcacactctgcattccagggacacgtggctgaagaaagcaagagtggtgaggcttggagaagtaccctacaagatggtgaaaggcttttctaaccgtgctcggaaagcccgacttgctgagccccagctgcgggaagaaaatgacctgggcctgtttggctactggcagacagaggagtatcagcccccagtggccgtggacgggaaggtgccccggaacgagtttgggaatgtgtacctcttcctgcccagcatgatgcctattggctgtgtccagctgaacctgcccaatctacaccgcgtggcccgcaagctggacatcgactgtgtccaggccatcactggctttgatttccatggcggctactcccatcccgtgactgatggatacatcgtctgcgaggaattcaaagacgtgctcctgactgcctgggaaaatgagcaggcagtcattgaaaggaaggagaaggagaaaaaggagaagcgggctctagggaactggaagttgctggccaaaggtctgctcatcagggagaggctgaagcgtcgctacgggcccaagagtgaggcagcagctccccacacagatgcaggaggtggactctcttctgatgaagaggaggggaccagctctcaagcagaagcggccaggatactggctgcctcctggcctcaaaaccgagaagatgaagaaaagcagaagctgaagggtgggcccaagaagaccaaaagggaaaagaaagcagcagcttcccacctgttcccatttgagcagctgtgagctgagcgcccactagaggggcacccaccagttgctgctgccccactacaggccccacacctgccctgggcatgcccagcccctggtggtgggggcttctctgctgagaaggcaaactgaggcagcatgcacggaggcggggtcaggggagacgaggccaagctgaggaggtgctgcaggtcccgtctggctccagcccttgtcagattcacccagggtgaagccttcaaagctttttgctaccaaagcccactcaccctttgagctacagaacactttgctaggagatactcttctgcctcctagacctgttctttccatctttagaaacatcagtttttgtatggaagccaccgggagatttctggatggtggtgcatccgtgaatgcgctgatcgtttcttccagttagagtcttcatctgtccgacaagttcactcgcctcggttgcggacctaggaccatttctctgcaggccacttaccttcccctgagtcaggcttactaatgctgccctcactgcctctttgcagtaggggagagagcagagaagtacaggtcatctgctgggatctagttttccaagtaacattttgtggtgacagaagcctaaaaaaagctaaaatcaggaaagaaaaggaaaaatacgaattgaaaattaaggaaatgttagtaaaatagatgagtgttaaactagattgtattcattactagataaaatgtataaagctctctgtactaaggagaaatgacttttataacattttgagaaaataataaagcatttatctaaaaaaaaaaa

SEQ ID NO: 38 - DNA, 6137 bp, Homo sapiens
  source 1..6137: mol_type = other DNA
gtcttttgtccctcggcggacaccgtttgccagccaaagctatgtctgcgcgctcaccgacttcatagggtgccgaattcttttttccccaggcttgccatggctagtcgaggggctcggcagcgcctgaagggcagcggggccagcagtggggatacggccccggctgcggacaagctgcgggagctgctgggcagccgagaggcgggcggcgcggagcaccggaccgagttatctgggaacaaagcaggacaagtctgggcacctgaaggatctactgctttcaagtgtctgctttcagcaaggttatgtgctgctctcctgagcaacatctctgactgtgatgaaacattcaactactgggagccaacacactacctcatctatggggaagggtttcagacttgggaatattccccagcatatgccattcgctcctatgcttacctgttgcttcatgcctggccagctgcatttcatgcaagaattctacaaactaataagattcttgtgttttactttttgcgatgtcttctggcttttgtgagctgtatttgtgaactttacttttacaaggctgtgtgcaagaagtttgggttgcacgtgagtcgaatgatgctagccttcttggttctcagcactggcatgttttgctcatcatcagcattccttcctagtagcttctgtatgtacactacgttgatagccatgactggatggtatatggacaagacttccattgctgtgctgggagtagcagctggggctatcttaggctggccattcagtgcagctcttggtttacccattgcctttgatttgctggtcatgaaacacaggtggaagagtttctttcattggtcgctgatggccctcatactatttctggtgcctgtggtggtcattgacagctactattatgggaagttggtgattgcaccactcaacattgttttgtataatgtctttactcctcatggacctgatctttatggtacagaaccctggtatttctatttaattaatggatttctgaatttcaatgtagcctttgctttggctctcctagtcctaccactgacttctcttatggaatacctgctgcagagatttcatgttcagaatttaggccacccgtattggcttaccttggctccaatgtatatttggtttataattttcttcatccagcctcacaaagaggagagatttcttttccctgtgtatccacttatatgtctctgtggcgctgtggctctctctgcacttcagaaatgttaccactttgtgtttcaacgatatcgcctggagcactatactgtgacatcgaattggctggcattaggaactgtcttcctgtttgggctcttgtcattttctcgctctgtggcactgttcagaggatatcacgggccccttgatttgtatccagaattttaccgaattgctacagacccaaccatccacactgtcccagaaggcagacctgtgaatgtctgtgtgggaaaagagtggtatcgatttcccagcagcttccttcttcctgacaattggcagcttcagttcattccatcagagttcagaggtcagttaccaaaaccttttgcagaaggacctctggccacccggattgttcctactgacatgaatgaccagaatctagaagagccatccagatatattgatatcagtaaatgccattatttagtggatttggacaccatgagagaaacaccccgggagccaaaatattcatccaataaagaagaatggatcagcttggcctatagaccattccttgatgcttctagatcttcaaagctgctgcgggcattctatgtccccttcctgtcagatcagtatacagtgtacgtaaactacaccatcctcaaaccccggaaagcaaagcaaatcaggaagaaaagtggaggttagcaacacacctgtggccccaaaggacaaccatcttgttaactattgattccagtgacctgactccctgcaagtcatcgcctgtaacatttgtaataaaggtcttctgacatgaatactggaatctgggtgctctgggctagtcaaagtctatttcaaagtctaatcaaagtcacatttgctccctgtgtgtgtctctgttctgcatgtaaactttttgcagctaggcagagaaaggccctaaagcacagatagatatattgctccacatctcattgtttttcctctgttcaattatttactagaccggagaagagcagaaccaacttacaggaagaattgaaaatcctggtactggatggctgtgataagctgttctccacactctggcctggcatctgagaactagcaagcctctcttaggccatatgggcttctccaccaaagctgtttggcagctcctagcagaccttcttattgaaatcctcatgctgaaaatgaacacagcctagttgccaacccacatgtccttttcacctccagcaagactaagcttctttaaagcacttcacaggactaggaccctgtcctggagctatctcaggaaaaaggtgaccatttgaggaactgtgacctaattttattataatgatgcctctaattttcatttcctttacaaccaactgtaactataaggttgtattgcttttttgttcagttttagcatgctattttttgaattctagactcctccatgtgaagatatcaacagacaaaactacaactgtataggacatatttggagaaaattctatcaattgatacatttggatgacatcacatttttaagtaatgtaatctgaggccattgctgaggaaattaagaattttcctttttttttaaccacccccagtgaaaaggatcagtgtatatttatagcacctattttttagttctgtctgttgtgaggcacatcctgcatggggcacttctagtcaaataggcaatgataaggacctaattaaaatgtgataagtgtatactattactttaaaagcctttacagtcagtacttcagtttacaaggcactttcacagcatctcgtttgatcctcacagtcacaacatgtggtagacaaggcaggtgatttttatccccattttacagataaggaaacaggctgcgggtggggagtgaggggaggtaaagatagttagttgcctaaggtcacacagccagtaagtaatagagctgggactggaacccaggtttccttactctcatctattgctcctccatattcctcactcaaccatgaaaacattacttgaaaggactgatgaggttaaccagagacctaactgatattgtaactttctattttaaggaagaattgtgtctgtatttgagttctttggagcctccagtctgcctgtgtgttagaccagcacagcagtgctgtgtgatgcagcctgacctgtggcaggaaagtagtgcttctgtttggaagtcatgttcttttgcagccacacaggatccaaatatcagtactattcctgtagtcaatctggggtcacattataggtgccttatttccctaagggtaactgatctgaatatctgcaaataggatgaatctatttttcagaagttccatctttcatttttctttttttttttgagacagagtctcattctgtcgcccatgctggagtgcagtggcgcgatctcggctcgctgcaacctctgcctcccaggttgaagcaattctcatgcctcagccacccgagtagctgggattacaggcatgcgccatcatgcccagctaatttatgtatttttagtagagttggagtttcaccatgttggccaggctggtcttggactcctgacctcaggtcatccacccgcctcagcctcccaaagtgctggtattacaggcgtgagccaccgcacccagccccatctttcattttcaaagagaagggcattctaataggaactggtgccaagagagaagaaaagaagtgataacagaagaaatggctagttacaatattaaaaagctcctctttgagatctcctctgcaggaatatcagagacggagttgaagcgctggagaggtaataggtctagacagtacagaacaataactggggagtgtgtgaggatagactgggctcccccttgcttgaaagatctctggcatttaattctcaattcttgattactattttccagtgtaaaactagcacatatgatctgactacaggacagagaattttaagtgaaacatttgccttacttgcagtaataatgtgctgttcttcacagtagctaaggccctctatgtttcccagaggtaaataagaatccaggaatggaggtccatctgtgatgaatggcttttttctaatcaaagtagtataatgctgttttatctgttttgtcatcttgtttttttttttttttaaaaaaacaaaaccttaattataatatagcgcaaagaaaggccaggactgatgcagggattccttggaaatatcagttcctatcacttttaaaacctgattttggatctctctgttctatgtatgtctttagtgagagcacaatacatggcagaacgctgtgccaaatgttataggtaaggaatatagaaatgaatgttttttgttgtgaaggtgttttcatgtgatattttataaacacattttaaaaaatctccatcactttttagtataggaaggatagctttgcctgggaaaaacagtttcaacacacctgctcagagtagcagttctccctcaaaaaagcagtgttcagcctgcactgactgttctgcttgccaaaaggaggaagcatgcaagatacttatttctccatagattgtggagtatagagggatgtgggactacagattattattttttttccccgagacagagtcttgctctgtcgcccaggttggaacacaatggcacgacctcagctcactgcaacctctgtctcccgggttcaagcaattctcctgcttcagcctcctgagtagctgggattacaggcacacaccaccaccgcactcagctaatttttgtatttttagtagaggtggggttttaccatgttggccaggctggtcttaaactcctgaccttgtaatcatcccgcctcggcctcctaaagtgctaggattacaggcatgagccaccgcacccggcccagataatttttaatagcctttgatcatggggtgagtgagggagtaggtatacttggcaaatgcatggttctctgatttctagctctaaagcagccttatctgaatccccaaatcttgtgatgctgagtaccattactgaaccagtctgcacggtaggcatctgctaccaaaatttacctcctacctggtaggtgtcatctgataagaaagaagacaggttattttaattttttgagataatcacagaaaattgcagcccatactctttattaccgaattcaagtttggaaatagaccctttgttttaaatcatgatgggtctttatcccaatcatttatctgggtcatttttccaactttggagttctaggaaagaaccttgaaaacctgatatgattctgcagcatgaggtctacggtgaccatttgggcaaagctccagtggcaatcatttattgtgttttgcatttcctgggatttattgaaataagaattcactgtgattatgtagtcttctggctagtatcaggcagctctgcttttaatttggttaattttattttctctgaagagggagaagaggtacaatttaatcttggcctccacaagcatattaaagctcacgtgttaatcagtgcattcttatgctcctacattaaatgccttgggtaaatggataaatggacatgtgcccagctttaattttttttgcaacagaaagatcagacttccgtatggcatcgttggatttcagaggctttctggtgtatctgtaaatctgaatgttgccttctgccagtctgtataaccaggtgattcatgctgcaaatgaaatcaggaagcagtaaagtgttaaagcaagagtattgtccaattcacttgtcttcctgatccttgtactttatttcacgtgtcggtgtttacattacatacttatatttcctgtgaaagaaagagttaaataaattgtagcagtttga

SEQ ID NO: 39 - DNA, 3531 bp, Homo sapiens
  source 1..3531: mol_type = other DNA
agcggagcgccaggcagcgcggagcggaggccaggcccacagccgctccgcctcccggcccgcagatccccgacggccgcaccgcgggctcctctggcccgcaagaacacgtgcatggcgtcctggggaaggcgctgagtgcggagtcgcggcgccgcacgcggcaccatggccctggagcaggcgctgcaggcggcgcggcagggcgagctggacgtgctgaggtcgctgcacgccgcaggcctcctggggccctcgctgcgcgacccgctggacgcgctgcccgtgcaccacgcggcccgcgctgggaagctgcactgtctgcgcttcctggtggaggaagccgccctccccgccgcggcccgcgcccgcaacggcgccacaccggcccacgacgcctccgccaccggccacctcgcctgcctgcagtggctgctgtcgcagggcggctgcagagtgcaggacaaagacaattctggtgccacagtcttgcatctggctgcccgcttcggccaccccgaggtggtgaactggctcttgcatcatggcggtggggaccccaccgcggccacagacatgggcgccctgcctatccactacgctgccgccaaaggagacttcccctccctgaggcttctcgtcgagcactaccctgagggagtgaatgcccaaaccaagaacggtgccacgcccctgtacctggcgtgccaggagggccacctggaggtgacccagtacctggtgcaggaatgcggcgcagacccgcacgcgcgcgcccacgacggcatgaccccgctgcacgccgcggcgcagatgggccacagcccagtcatcgtgtggttggtgagctgcaccgacgtgagcctgtccgagcaggacaaagacggcgccaccgccatgcacttcgcggcgagccgcggccacaccaaggtgctcagctggctgctgctgcacggcggggagatctcggctgacctgtggggcgggaccccgctgcacgacgccgccgagaacggggagctagagtgctgccagatcctggtagtgaacggcgcggagctggacgtccgcgaccgcgacgggtacacggccgccgacctgtcggacttcaacggccacagccactgcacccgctacctgcgcacggtggagaacctgagcgtggagcaccgcgtgctttcccgggatccatccgcagagctggaggctaagcagccggattcaggcatgtcctcacccaataccacggtgtcggtccagccgctgaactttgacctcagctcgcctaccagcaccctctccaactacgactcctgctcctccagccactccagcatcaagggccagcaccctccatgtgggctttccagcgctagagctgcagacatacagagctacatggacatgctgaacccggagctgggcctgcctcggggcacgattgggaagcccacacccccaccacccccacccagcttccccccgccacccccgcccccaggcacccaactgcccccacccccacctggctacccagctcccaagcctcctgtaggaccacaggcagctgacatctacatgcagaccaagaacaaactccgccacgtggagacagaggccctcaagaaggagctgagctcctgtgacggccacgacgggctgcggaggcaggactccagccgcaagccccgcgccttcagcaagcagcccagcacgggggactactaccggcagctgggccgctgccccggcgagacgctggccgcacgcccgggcatggcgcacagcgaggaggtgcgtgcccgccagcccgcgcgcgccggctgcccgcgcctcggccctgccgcccgcggctcactcgaaggcccctccgctcccccgcaggcggcgctgcttcctgggaaccatgttcctaacggctgcgccgcggaccccaaggcgtccagggagctgccaccgccgcccccaccgccgccgccgcccctgccggaggccgcgagttcgccaccgccggccccgcctctgcccctcgagagcgctggccctggctgcgggcagcgccgctcctcctcgtccaccggcagcaccaagtctttcaacatgatgtccccgacgggcgacaactcggagctactggctgagattaaggcaggcaagagcctgaagccgacgccccagagcaaggggctgaccacagtgttctcaggcatcgggcagccggccttccagcccgattcgccgctgccttctgtgtcacctgcactgtcaccagtccggagccccacaccgccagctgcggggtttcagccgctgctcaatggaagcttggttcccgtgccgcccactactcctgcgccgggagtgcagctggacgtggaggctctcatccccacgcacgatgagcagggccggcccatccccgagtggaagcgccaggtgatggtgcgcaagatgcagctgaagatgcaggaggaggaggagcagaggcggaaggaggaggaggaggaggcccggctggccagcatgcccgcctggaggcgggacctcctgcggaagaagctggaagaagagagggagcagaagcggaaagaggaggagcgacagaagcaggaggagctgcggcgggagaaggaacagtcagagaagctgcggacgctgggctacgatgagagcaagctggcgccctggcagcgacaggtcatcctgaagaagggggacatcgctaagtactagaggccgcagactcctgtccgcagcctcgcagctccgtggggccctccgccccagccccagccagccaggccctggtggaaaggctgggagccgcacagccctcccctcctgcgctggaaaccctccctgacccccaccctggccccccgtatccccagcccttggcaacactggagtgcacacgccgccacggttgcccagaaaaagtgcccaagctgctgacgcaaacaacaacaaatgctgcttatttgcatgccgacttacatatatttgcatgttcgttgactatcaaagagtgcagagctctccccagccccgtgggtggtgactttgttttcctgcggggctcagccccctccaggatgcagccccctcccccgcaccccggaaccggcgtcgctggcgcatcctgggtggaggcaggccccgagctcggggaaggggttttcccttcctctctgacccagatctgcgcgcggcctagcccgggcctcatttcttatccccgccaagggtttcctctcagtcatttgtttaccagaaacatgaaaactgcctgtctggccgggccgcacttgtggcccccgggaccccacctctggccccacctccctcaagtctgcgccccgtccccagccagacccactcgctgccgggaccctttcactgccccggtggagtgaatagaggatgaggggccctgaccctgtgtctccaactgctgcaccccatcccgaccctgtctccgccacctcgcagccccattaaagcgctctcatctgggctccggttcactca

SEQ ID NO: 40 - DNA, 2272 bp, Homo sapiens
  source 1..2272: mol_type = other DNA
ttgggcggtggaccgcccctcggccccggggtaggctgacacgggagggtcctcagctaaagccaaaagcagatcaaagtggtgggactcgcgtcgcggccgcggagacgtgaagctctcgaggctcctcccgctgcgggtcggcgctcgccctcgctctcctcgccctccgccccggccccggccccgcgcccgccatggagaagactgagctgatccagaaggccaagctggccgagcaggccgagcgctacgacgacatggccacctgcatgaaggcagtgaccgagcagggcgccgagctgtccaacgaggagcgcaacctgctctccgtggcctacaagaacgtggtcgggggccgcaggtccgcctggagggtcatctctagcatcgagcagaagaccgacacctccgacaagaagttgcagctgattaaggactatcgggagaaagtggagtccgagctgagatccatctgcaccacggtgctggaattgttggataaatatttaatagccaatgcaactaatccagagagtaaggtcttctatctgaaaatgaagggtgattacttccggtaccttgctgaagttgcgtgtggtgatgatcgaaaacaaacgatagataattcccaaggagcttaccaagaggcatttgatataagcaagaaagagatgcaacccacacacccaatccgcctggggcttgctcttaacttttctgtattttactatgagattcttaataacccagagcttgcctgcacgctggctaaaacggcttttgatgaggccattgctgaacttgatacactgaatgaagactcatacaaagacagcaccctcatcatgcagttgcttagagacaacctaacactttggacatcagacagtgcaggagaagaatgtgatgcggcagaaggggctgaaaactaaatccatacagggtgtcatccttctttccttcaagaaacctttttacacatctccattccttattccacttggatttcctatagcaaagaaacccattcatgtgtatggaatcaactgtttatagtcttttcacactgcagctttgggaaaacttcattccttgatttgtgtttgtcttggccttcctggtgtgcagtactgctgtagaaaagtattaatagcttcatttcatataaacataagtaactcccaaacacttatgtagaggactaaaaatgtatctggtatttaagtaatctgaaccagttctgcaagtgactgtgttttgtattactgtgaaaataagaaaatgtagttaattacaatttaaagagtattccacataacttcttaatttctacattccctcccttactcttcgggggtttcctttcagtaagcaacttttccatgctcttaatgtattcctttttagtaggaatccggaagtattagattgaatggaaaagcacttgccatctctgtctaggggtcacaaattgaaatggctcctgtatcacatacggaggtcttgtgtatctgtggcaacagggagtttccttattcactctttatttgctgctgtttaagttgccaacctcccctcccaataaaaattcacttacacctcctgcctttgtagttctggtattcactttactatgtgatagaagtagcatgttgctgccagaatacaagcattgcttttggcaaattaaagtgcatgtcatttcttaatacactagaaaggggaaataaattaaagtacacaagtccaagtctaaaactttagtacttttccatgcagatttgtgcacatgtgagagggtgtccagtttgtctagtgattgttatttagagagttggaccactattgtgtgttgctaatcattgactgtagtcccaaaaaagccttgtgaaaatgttatgccctatgtaacagcagagtaacataaaataaaagtacattttataaaccatttactatggctttgtaacaattgcatacccatattttaagggacaggtgaatttactactttctaaagtttattgatacttcccttttatgtaaaatgtagtagtgatacctatatttccacattgtgcattgtgacacacttgtctagggatgcctggaagtgtataaaattggactgcatttcttagagtgttttactatagatcagtctcatgggccatctcttcctcagatgtaaatgatatctggttaagtgttatatggaataaagtggacattttaaaactagcaaagttaaaaaaaaaaaaaaaaaaaa

SEQ ID NO: 41 - DNA, 4510 bp, Homo sapiens
  source 1..4510: mol_type = other DNA
gcagagggggcggagagcgcccccgggggcggggcacgcaagtgacggcggcgcgggtggtggagcgctgggcggccaggctccctggctggccggtttgggcgtctgggccgtgaaggtgggacctcctgttccgggccgcaagtttccctctccagccgcccgccgttcgtagcatgtcccccagaactcggggagcgcaggcaggacaggcttagagaagacgcggtccccagcgcttgggccacggacgtcccaccccgctcctctgtcgctggagaaccgccgggccgagccactgggagaagcaggccagagccttccagggcctccggcccgtggacccgaggaggatgagctggctttttcccctgaccaagagcgcctcctcctccgcggctgggtcccccggtggcctcaccagcctccagcagcagaagcagcgcctgatcgagtccctccggaactcacactccagattgcttcctccacagtttcctcaggaaaaaccagtgatcagtgtttatccaccaatacgacatcacttaatggataaacaaggagtgtatgttacctctccattagtaaacaattttacaatgcactcagatcttggaaaaattattcagagtctgttggatgagttttggaagaatcctccagttttagctcctacttcaacagcatttccttatctatacagtaacccaagtgggatgtctccttatgcttctcagggttttccatttcttcctccatatcctccacaagaagcaaacaggagtatcacttctttatctgttgctgacactgtttcttcttcaacaacaagtcataccacagccaagcctgccgctccttcatttggtgtcctttcaaatctgccattacccattcccacagtggatgcttcaataccgacaagccaaaatggttttgggtacaagatgccagatgtccctgatgcatttccagaactctcagaactaagtgtgtcacaactcacagatatgaatgaacaagaggaggtattactagaacagtttctgactttgcctcaactaaaacaaattattaccgacaaagatgacttagtaaaaagtattgaggaactagcaagaaaaaatctccttttggagcccagcttggaagccaaaagacaaactgttttagataagtatgaattacttacacagatgaagtccactttcgaaaagaagatgcaaaggcagcatgaacttagtgagagctgtagtgcaagtgcccttcaggcaagattgaaagtagctgcacatgaagctgaggaagaatctgataatattgcagaagacttcttggagggaaagatggaaatagatgattttctcagtagcttcatggaaaagagaacaatttgccactgtagaagagccaaggaagagaaacttcagcaggcgatagcaatgcacagccaatttcatgctccactatagattttcctggaaacatgaactgccaagagaggaatgggacacaaaaccaaacactgttttatatttatggtttgcaaactggcatttcatcagtggctaaattcacagatatcctatatagattgtatacagaactgagactgattttgtaccgattagaatgattgctatgatctttgagaaatttttctgcactatttgcactgaaatgtttatttattgttgataaattgtatcatatttaagttccactgctgttcctcttaccttgattaaatgcctatgcatgtacttttagctagtttttaatattttataaaacttcatttaaatttgtatttttaacttgaagttccatttctttatcaaggatggtatttagatttttttcctcttaaccttttttcaaaaactattttcaactgtgaggaaacccttatttttctttctttgtggataaaactttcaaaagcaatttaagatattcatagtgttaggaaacaccaaacctgcctatgtgccatctcacaaaagaaacttttaatacctacaataaatcaaaagaataaaccagctgttcttatatattgtttcatttttaaaactaaagatgcatttaagaagcaatacaagtaaatattttacctaataggaaaaaaaaaagttgcctttcatttaaaccattccaacagaaattcttatgctaatttaaaacatatatatatctggtaggtttgtggttggataggttttctaaattcctaatgttaaaaacaatctttatgttaatatacactaaatctatacacaaaaaaagtcagtgaacttttctgacctttactgtgagttaccttttcctaagaggaaagctatagtaataagtaaaatttaatttttaggcaatcctgatttttaatgaatttaattgagtgttcttgtatactacattgagcagtttgcttctataccgtgtcacaaaattcatgtatttcttgagaagccctaaaagctcataaaggaaaatgccgtgaactatgtagctcaggcttggtaaggtgccatctaaattacaaaacaaactaatgcataattttgcttaaatttcatcccagtatgattgtcttcccaacaccagcatatagtatagattgtctgtcttttttatattttttagttcttcctgtacatgtttttggcaataaagttataggaagaacaaaattattttgttagaattaaaacatgcttaatatttagtctgtttgtggagggcaggtattcacgtggactgagatacaatgttggatacagaaaataactttcattgtcttcctgacactgtgctaaggacatgctgttaaagcttcaaagtgaccagatgaggaaggaataattaattattactcctgatttgtagataactgaggtaagagtgtttcaaatttatgatagtcttttgggtattcagaaacctttccttatactgcactggccaccagagcttaattttcccagcagttacagcaatgggagatagaacagtctcaatcttttgccaaccatcaggttcctagaaaccaggtaggtgtatcccataacaagggaggagcataccacagcccctcatttgattaattcatttgatctatctatgttattaagtacctactaggaataaggcattgtggaaatactatacaaagataaacattgtttagatgcttatctactttccttttcaccagaaaaacagaaaaaaaagaaacattttcttacagagtaaaaatgttctacataatcacatgagtagttcatctcagtgttttttattctttaaagttgaactatcccagtttcattctataccattcattggataaccttgttacaacccagtcatgaaacagagcagtgtgatcagttatctgcatttaacaaatagacaaatcagtttacataaaggttatgtatgtcacccacgatgaaaagaatctgcatttgaatatgcccgtatgaatgtgggttctgtttttgcaacagagattaagtgaccattttttctaattttatggctatatattttcttcataaaaattggtcacatcggagaagcagtgccacaggaaaaatgaaatgcatgtgaaagtttgtattctgattttacaagatgagatagaaatcagaattaaagaggaatacttaggagttactaggctaatcagtgtacgaatttgtcataggtagagatttaaaggttaatatcttaaaatagaagaaaattctaaatcaatcaatcagtgagatataaactaaacagacccacttcaaagttgaaagaaatttctaggcataaattgagactaggaaatttatatcagaatagagggtgcttgacacatatatatgcttaaattgaaggacagctcagattcatttttaggagaagaaagtaaactaatgtgctcttaaagaataaaaatttattctatggtttctgtctctgatcatcaccttccattctataaaaagctcagttactgatttgctgggtcatggtcaaaattcttacctatttatttcatatcaactttaaaaaataaattacttgcattctatatattactaattgggaagtaatatgcctcaaatcagttttatactggattattccctatgctttaaaccactgctctcaataaaacacttcctgattaatgtttgattattagatattttagtcttgttggggatattttagtcttgttgggttagccatgctctgaagaatctgtgaaagtacagtaaagttttaataagcaataaatgtaaccttttatataaatctcagtgctaggttaacttctaataagcagacgaacatgttacataaattataatgtctgtcttgtaaaaaagttgaggggactaaaagtttatgactctgatatggaagttgtcatattaaaaaactacattttaaaacatcaaatatttatactatttgcttttcaaataaaagcatagtgctgtttggcata

SEQ ID NO: 42 - DNA, 11062 bp, Homo sapiens
  source 1..11062: mol_type = other DNA
gcagatcgggagcggtgccgagaaaaatttccttactagatgacatttcatcgcaatgtccgatcgtttggggcaaattaccaagggcaaggatgggaaaagcaagtactcgactctcagcctgtttgataagtataaaggaaaatcagtagacgcgattagatcctcagttattcctagacatggcttacagagtcttgggaaagttgctgcagcccggcgcatgccaccgcctgcaaacctgccaagcttgaagtctgaaaacaaaggaaacgaccccaacatcgtgatagtacccaaggacgggacgggatgggcaaacaagcaggatcagcaagacccaaagagttccagtgcgacggcctctcagccgccggagtcgctgccgcagccgggtttgcagaaatctgtctccaatttgcagaaaccgacacagtcaatcagtcaggagaatacaaattcagtgccaggtggaccaaagtcatgggcacagctgaatggaaagccagtaggacacgaaggtggtttaaggggctcaagccgactgttatccttctctcccgaggaatttccgacgctgaaagcagctggagggcaggacaaggctggcaaagaaaagggcgtcttagatctgtcgtatgggccaggaccaagcctccgccctcagaatgtgacaagctggagggagggcggtgggcgacacataatttctgccacgtctctgagcacctccccaactgagctgggcagcaggaactcgagtacgggagatggagccccctcctcggcatgtaccagcgattctaaggacccctctctccgcccggctcagcctgtccgaaaaggggcttcacagttcatgggaaatgtataccacccacctacataccatgacatgcttcctgcttttatgtgttcgccgaagtcatcagaaaaccagggtacagtggaacgaggctcttttccccttcctcagctccgccttgaacctcgagttccttttagacagttccagatgaatgaccaagacggaaaagaaaacaggctgggattgtctcgcccactccgcccactaaggcagctggtggagcgggcaccacggcccaccattatcaatgcggaaaacctgaagggccttgacgatctggacgccgatgccgatgatggctgggcaggcctccatgaagaagtggactattctgagaaactgaagttcagtgatgatgaagaggaggaagaagttgtgaaggacggcaggccaaagtggaacagttgggaccctaggaggcagcggcagttgtcaatgagctctgcagacagtgcggacgctaagcggactcgagaggaagggaaggactgggctgaagcagtgggtgcgtcccgtgtggtccgaaaggcgccagaccctcagccaccgcccaggaagcttcatggctgggcaccaggccctgactaccagaagtcatcaatgggcagcatgttccggcaacagtccatcgaggacaaggaggacaagcccccaccaaggcagaagttcattcagtcagagatgtccgaggcggtggagcgagcccgaaagcgccgggaagaagaggagcgccgagcccgggaggagaggctggccgcctgtgctgccaaactcaagcagctggaccagaagtgtaagcaggcacgaaaggcaggtgaggcccggaagcaggcagagaaggaagtgccctggtctccaagtgctgagaaggcatctccccaggaaaacggccctgctgtccacaaaggctccccagaattccctgcccaagagacccccaccacattcccagaagaggcacccacagtgtccccagcagtggcacagagcaacagcagtgaggaagaggccagagaggctgggtcccctgcacaggagttcaagtatcagaagtcccttcctccccgattccagcgccagcagcagcaacaacagcaggagcagctgtacaagatgcagcactggcagccggtgtaccccccgccgtcccacccccagcgcaccttttacccacaccacccccagatgttgggcttcgatcccaggtggatgatgatgccttcctacatggacccacgtatcacgcccactcggaccccggtggacttctacccctccgccctgcatccctcaggactgatgaagcccatgatgccccaggagtccctcaatgggacaggctgtcgctctgaggatcagaactgtgtgcccccactccaagaaagaaaagtgacccccatcgactcaccccctgtgtggagcccagagggctacatggcactgcagagcaagggctacccgctcccgcacccgaagtcgagtgacaccttggctatggacatgcgtgtcaggaatgaaagctctttctctgcctcactcggaagggcagggggcgtaagtgctcagcgcgatctctttgaggagagaggggaggagtacttgagtgcttttgacaagaaggcccaagcagactttgacagctgtatctcttctcaaagaataggccaggagcttttgtttccaccccaagaaaatgttcaggatgcaggtgctcctgggggtcacacccaaaacctcaggtgttccccattggagcctgactttgtcccagatgagaaaaagccagagtgtggcagttgggatgttagccaccagccagagaccgctgacacagcccatggtgttgagcgggagacaccccgggaggggacggcctttaacatctcctcctgggacaagaacgggagccccaacaaacagccatcctcggagcctgaatggactcccgagccccggagctccagcagccagcacccggagcagacgggcaggacccggaggtcgggacccatcaagaaaccagtcctgaaagccctcaaggtggaagacaaggagaaggagcttgagaagattaagcaggagctaggggaggagagtacccggctggccaaggagaaggagcagagccccacggcagaaaaggatgaggacgaagagaacgatgcctctctggccaactcctccaccaccactttggaggacaaaggccctggccatgccacttttggccgcgaggccaccaaatttgaagaggaggagaaacctgacaaggcctgggaagccagacccccacgagagtccagcgatgttccccccatgaagagaaataactggatctttattgatgaggagcaagcctttggggtcagaggacaggcccggggccggggccgtggtttcagagagttcacttttcgtggtcggcctgctggcggaaatgggagcggcctctgtggtgggggggtcctgggggcccgcagcatctactgcagcagtcagcgcagcggccgtggccggggcctgcgagagtttgcgcggccagaggactgccccagagccaagccccgacggagagttgccagtgagacccatagcgagggctcagagtatgaagaacttcccaagcgccgccggcagaggggctccgagaacgggaatgaaggctcgctcctggagagggaggagagcaccttgaagaagggcgactgcagagattcttggcggtccaacaaggggtgctctgaggaccacagcggtctagatgccaagagccgaggccctcgggcctttgggcgagccctccctccccggctgagcaattgcgggtatggacggagaaccttcgtctccaaagagtcaccccactggcagagcaaaagtccaggcagctcttggcaggaatatggcccttccgacacatgcggatcccggcgacctacagacagagactatgtcccagattcctacagacaccctgacgcatttggtggccggggctttgaggacagccgcgcggaggacaagagatccttcttccaagatgaacacgtggcagattctgaaaatgcagagaaccggcccttcaggagaaggcgccccccacgccaagataagccccctcgattccggcgcctccggcaagagcgggagtccctgggcctgtggggacccgaggaggagccccacctgctggcaggtcagtggccaggcaggcccaaactgtgttctggggacaagagtggcactgtgggccgcaggtcccctgagctctcctaccagaactcctccgatcacgccaatgaggagtgggagacggcctccgaaagcagcgacttcagcgagcggcgggagcggcgggaaggccctgggtccgagcccgactcccaggtggatggtggcctgtcgggggctagtttgggtgagaagaaggagctggccaagaggagcttctccagtcagagacccgtggttgacagacagagccgaaagctggagccgggagggtttggggagaagcccgttaggccaggtggtggtgacacctcccctcgctatgagagccaacagaatgggacgcctttgaaagtgaaaagatccccagacgaggccttgcctggaggtcttagtggctgcagcagtgggagtggccactccccctatgccctggagcgggcagcccatgccagtgctgaccttcccgaagcctccagtaaaaaggcagagaaggaggccaagttggctgctccgagggcaggtgaacagggagaggccatgaaacagtttgacctgaactatggaagtgccatcattgaaaattgcgggtccagccccggggaggagagtgaggtgggttctatggtgggcgaaggcttcatcgaagtcctgaccaagaagcagcgccgcctgctggaggaagagagaagaaagaaggagcaggccgtgcaggtgcctgtcaaaggtcgaggcctttcctcccgtattcctcctcgatttgcaaaaaagcagaacaacttatgtctggagcaaggtgacgtgaccgtgcctggcagcagcctgggcactgagatctgggagagcagcagccaggctctccctgtgcaggccccagccaacgactcctggaggaaagctgtcactgccttcagcagcaccgagactggctctgcggagcagggttttaagagcagccagggagatagtggcgttgacttgagtgccgagtctcgggagtcgtctgcgacctcctcgcagcgcagctccccatatgggactctgaagccagaggagatgagcgggcccggcctggcggaacccaaggccgacagccacaaggagcaggctccaaagccatctgagcagaaggattcagaacaaggctctggacagagcaaggagcacagaccaggacccatcggcaacgagcgttctctgaaaaacagaaagggctcggagggggccgagcggctgcaaggggctgtcgtcccgcctgttaacggggtggagattcacgtggactccgtgctgcctgtgccacccattgaatttggagtcagtccaaaagactccgatttcagcttgccacctggttctgcctctggtcctactgggagtccagttgttaaacttcaggatgccttggccagtaatgcagggttaacacagagtatccccatcctgcggcgggaccatcacatccagagggccatcggtctctccccaatgtccttccccaccgccgaccttactctgaagatggagtctgcgcgcaaggcttgggaaaactcccccagtttgccggagcagagctctccaggcggcgctggctcaggcatccagcctccatcctctgtgggtgcctccagcggggtcaactacagctccttcggtggagtgtccatgccacccatgcctgtggcctctgtagcaccttctgcttctatgccaggcagccacctcccgcccctgtacctggatggccatgtgtttgcaagtcagccccggctggttcctcaaacgatacctcagcagcagagttaccaacaggccgccgctgcccagcagatcccgatctcccttcacacatctctgcaggcacaagctcagcttggactgaggggtgggcttcctgtgtcccagtcccaggagatcttcagctccttgcagcccttcagatctcaggtgtacatgcaccccagcctgtcaccgcccagcaccatgatcctctctgggggcacagccttgaagcctccatactcggcgttcccaggcatgcagcccttggagatggtgaagccgcagtctggctcaccctaccagcccatgagcgggaaccaagccctggtctacgagggccagctcagccaggctgctggcctgggtgcctcccagatgttggactcccagctcccacagctgaccatgccactgcctcggtacggctccgggcagcagccactgatcctgccccagtctattcagctgccacctgggcagagcctctccgttggggccccccgaaggattcctccgcccgggtcccagccgccagtcctgaacaccagcagagagccctctcagatggagatgaaaggcttccactttgccgacagtaaacagaatgtcccttcaggaggccccgtgccatcgccacagacctacaggcctagctctgctagccccagtgggaagccctctggatcagcagttaacatgggctctgtgcagggacactacgtgcaacaggcaaaacaacgagtggatgagaaacccagcctgggagccgtgaagctgcaggaggccccctcggctgcctcccagatgaagcgaaccggagcgatcaagcctcgggctgtcaaagtggaggagagtaaggcctgacagtgcctggctgccacctcgcctctccctactgaggacggtgccgccatgcggcctcgacacagccgacactcgggagcctcaccagatccaccgtccaaatgcgtggcccagactgagagacctccctcctctccactcccgaaagctccgttgtcaaccagcttgcacccgtggatatatggcattgacccgcttgctttgatacgaaacaaaaaagcagacgactccttcatcccatctgctcctaccgtgactgtggagtgacgcctcctgtgcagtgcagatttgccctccctgcctcctccctgtcctgccgcgcagccagggcgccttctcagcagtgcttccggcccagccgcccatccctaggcacagtgatttggcagcagggtcattttactttgaggctttttgttttaaaatgtagccaaggtttttacaaaggggaaaggaaaagaaaacaaaaacgcaagctccatgtgtatagctgaacttttatatgtttcttgccagcccctccgctcccttccatctctagcctctgtcctgtttagtttgatacgtcactgcagtaccttaagaggtgactcttaagaatgcatcccctcctgattcctcagctggttcacccttgaggttatttgcaaaaagaaaaggaggttcttgagggcaccgattgcgagcattctggtgcctggctccccgcctgggaagcgatggggtgctcagagcagcaggcaggttgggggagggggggggtcatagttgggttccagctcctggcttgatgagcccagggcgcttacaggcagcccatgaagttgatgacagttttagcatgagaatcacacagggtccctgtcctgggctcctctaaagccagtggatgtgctgggcaccagagacaaatcatggagatggctgctggtggctcccaggttggcccagatggggtgagctgacataccacaggcccatcccaggccccgtgggctctgcttctggggctccataccctgccctgcaggggtgctgtgtttttcacacatttctttccctgaagccttctgtaacctgtcattttccttccttcctcttccggagcctgctgctttctctggacctgtctccacctcccacacagctcatcgtgaacaccacttggtgatggagggagtggacccgtgtgtggtccccaagtgaggccactgggagtttgtccttttcctcctttgcttcactcccagcagcagacccaggttgtcaggacaggagggcctgagctaagcagtaggcatcagtctcgtttgtcttcagacggcgggggcaggtccagggtgaggctgggtggagggctgaccaaggtccaaagggcctgcgcagcctccgggagggcagcttctccagccagaggcttgtgtgagccatcgtgtgctgggcttgtttttaagtaagaaacaaggaaatcactccagattctgtcattccaaggaaagggaaggggacagttcaggtttctcagctgttcttaggggtcactgagcgtctacctcctcctccagaggaggctggctcagaacacctagaggagggggccggggatgcaccccccaccagaggctgccttcagcgtctcacgggtgcaggacagcgctcaggcttgggctctaagctctgtgtctagtgtagaacatggggaaggagcatcttaggaactgctgaagtaacttcttactgctctcacaattctaaggaagcgggagaacggcctcctaccaacagcgcccaccccagagctgcctgggaaagggcagttttactgaaaggtgctttactgttcacctgcatctttcagcagctcccctcctgccctcacctggtcttttccctctttatcccaagcctttatgcttgagtcccttccccaggggctgcccacccgacagttccaggcattccctacctgagcttcttgtctgcttttccttctcccactgcaagcggctgcttgtggggcctgggatgagccctctctgtccccaccggccctccttgccaagccattcctgggtgagttcaggcctgcgggagccacacattcatctccacctggacacttgagccgcatggccagacccctcccacctgatgcggtggtgcgtgtgatttgtcaaaagaaagccttctggatgctgttaagatgtacccttcaggtgaacctggtatcagacccacagtacttgctgtttgagaaaaaataaaaacaaaaaggtcacctgttctccagcccttttctcttacctggtatttccttcctttctcctcccccaccccaaataaaaaaacaaaaaacactagaatttatttatatgtattgatgttgtaggtctaggtgaaaaaaaaagaagtaaatgtttcactgctctatttatatataatgtctgaattaattctgtgcaggaaaggccaggaaattgcatgtgaagttcggtgcagtcaccacctgtgtgtgacctgagctgcagtctcttcgctgagatgcaggttttaaatgagacttggggggctgagggcaggcctcaggcctcccagcgccccaacccctccttggtctaatgaaatgcagttcttagtgcagagatgttttaaggtgcaatatatctcttcctttcccgtggttttagagccaagctcaaggtagtaggacgtagggtcttattttgttttcaaacccccatcctcagagcgcagatacatgcagaggcttctgccaggataccacggggccttagtgggaacaggtggagaccagcacttccctttcctgctgctgaggtagggattggggggtcagaacccactcacttttgcctgttaaagttgccctcctgacgctggcagctctgccttggtcactggggatgcggctcgttgctcagccaccagtggccttgcggtattgtccaccatccactagagtgggatgaagtccagagtgtgggtatacatctcagatgcccatctacccactggggacttcaatgccagctgcatttggtttggttttcttaactgttggcttctccccacagcgttttttgtttttttttaaacattcatattgttttcaaacttggaattcatagacactctggctctaggttccttaagggggaaaacaaaagatgactttatttcacattcaagaaaatcagttcagttccaaagctgtggtccttccagccacttctagggacactggggaaccttgttaaacgttgacatcagtgctctccagccgtgctgtcaccctcctatcttctggatctgccttcgcgatggtcagtgacagcttctggaagctgagcacacacaggtgcacagccatgctgtggtctggcctgctacggcagcatggcagctctggtggagccttctcccttgccatttggttcccctgtgccaagtagctgcaggctgcccctcaaatcttcatttgtcccttttcacttcctgcagaacaagcctgggttagagggtctgctggaaatggcctttgaagaccaaggataccaggatgtgtgcactctgtcgtgttctgtgatgaatgggaaacgtaggcttccagaaagccagctctcttctgaaatgtgacggacctaagcaggaagtcatccaggacaggagtggctcagtgttggggatggacgctgtcgcccagccatgctccaccagggccaccaatgtgtagttggctggtggtcttcgggcatgtgagacctgctcttcactgtttccaccccacttggtggcctccaggatggtagtggcaccctcagagccccatcttcagcatgttctgaagcctcagagtggaaattcctgctaaggctctgtgtggacgcctttctcccgtgatctaaaggggacactgtactcaagcttttgacctcatgccttgtgtagtaaaaaaggatttgggggttttgtttggttcctgagagggttgtgttttgtttttgtttccttttgtttatgttttggcctttcctctttgtctttccatgtagaccagatatttgaaagggcagacgatggctagaggtgtaatgtgcagcttgtttatacggtattttgggaaacttaccttggatgggaaatcgaatcgtggattcaccaggccggtgctggcacactcaccctcgccctttccctccggttcagtacctattgtttctcctttcaaatatgtgattgtactagctctttccatatgaaagaattctccttatttaaataaaaaaagtttaaaaa

SEQ ID NO: 43 - DNA, 7689 bp, Homo sapiens
  source 1..7689: mol_type = other DNA
tcccacagtgcctggcccagaagccttgctaaatatttgaacaggattgcccaatacttttctgctgtgagaatgtaagatggatccagaagagcaggagctcttaaatgattacagatacagaagctactcttcagtgattgaaaaggctttgagaaattttgagtcctcgagtgaatgggcggatctcatatcttcacttggcaaactcaacaaggctcttcagagtaacctgaggtactccttgttgccaagacggctcctcatcagcaaaagattagctcagtgtttgcaccctgccctgcccagtggtgtccacttaaaagctctggaaacctacgagattatctttaaaatcgtggggaccaaatggctggccaaggacttgtttctgtacagctgcgggttatttcctctcctggcacacgcggcggtgtcggtgaggccggtgctgctcaccctgtacgagaagtacttcctcccactgcagaagctgctcctgcccagtctgcaggccttcatcgtgggcctgctgcccggccttgaagagggctccgagatctccgacagaacggatgctctgctcctgagactgtcgctggtggttggcaaagaggtgttttacaccgccctctgggggagcgtcctggccagcccgtccatccgcctccctgcctcagtcttcgtggtgggccacatcaacagggatgcccccggccgggagcagaagtacatgctggggaccaatcaccaactcacggtgaagtctttgcgtgcctccctgttggactcaaatgttcttgtgcaaagaaataatctggaaatcgttctgtttttcttcccattttatacctgtctggattccaatgagagagccatccccctcctcagatctgacatcgtgcgcattctctcagccgccacccagaccctactgagaagggacatgtccctgaacagaagactgtatgcatggttactaggctcagacataaaaggaaataccgttgtgccagaatctgaaatctcaaattcttatgaagaccagtcgtcttatttttttgaaaaatactccaaggatcttttagttgagggtttggctgagatattgcatcagaagttcatagatgctgacgtggaggaacgccatcatgcatacctgaagccttttcgcgtcctcatcagtctgcttgacaagccagaaatagggcctcaagtggttgggaatttgtttctcgaagtcatcagggccttttattcttactgcagagatgcccttggctctgatcttaaacttagctacacccagagtggaaattcgctgataagtgcaatcaaggaaaacagaaatgcctctgagattgtcaaaacggtaaatttgctgataacttctctaagcacagactttctctgggattatatgacaaggtgttttgaggaatgctttagaccagtgaagcagcgttacagcgtgaggaacagcgtcagccctccccccacggtctcggagctctgcgccctcctggtcttcctgctggatgtcattcctttggaactttactctgaggtgcaaacccagtatctccctcaggtgctcggctgcctggtgcagcctcttgctgaggacatggaggccttaagtttacctgaactcacgcatgccttgaagacgtgtttcaaggtgctcagcaaagtccagatgcctccttcctacctcgacacggagtccaccagcggaacctcgagtccagtaaaaggtgaaaacggcaaaataattttggaaacaaaggcagtgattcccggtgacgaagatgcttcgtttccccctctgaagtctgaggacagtgggatcgggctcagtgcctcgtcaccggagctctctgagcacttgagggttcctcgagtttctctggaaagggacgacgtttggaagaagggcgggagcatgcagaggacgtttctttgcatccaagagctaatcgccaactttgccagcaagaacatttttggagtacagctgacagcgtcaggagaagaaagcaagtccgaggagcctgcagggaagagggacagggatgggacgcagagcctggcagccaatgattccagcaggaagaactcttgggagcccaagcccatcactgtgcctcagttcaagcagatgctgtcagacttgttcacagcacgagggtctccattcaagacaaaaagttcagagtcaccatcgtcttcgcccagcagccctgccaggaaaaacgggggagaatgggatgttgagaaggtggtcattgacctggggggttccagggaggaacgcagggaggcctttgccgccgcctgccacctgctgctggattgtgccactttccctgtctacctgtccgaggaagagaccgagcagctctgtgcaacgctcttccagctgccaggagccggtgattccagttttccatcttggctgaagtccctcatgactatttgctgctgtgtgactgactgctacctccagaacgtggccatttccactctgctggaagtgataaaccattcccagtccctggcgcttgtcattgaagacaagatgaaacgctataagagctctggacacaaccctttttttggcaagctgcagatggtgacggttcctcccattgctccagggatattgaaagtcattgcagagaaaacagatttctatcagagggtggctcgtgtgctttggaatcagctgaacaaagagacccgggagcatcacgtcacctgcgtagaattgttctaccggctgcactgcctggcccctacggccaacatctgcgaggacatcatctgccatgccctcctggaccctgacaagggaacaaggctggaagctctgtttagattttccgtgatctggcatctgacaagagagatccaaggcagtcgagtaacatctcacaatcgctcctttgataggtccttgtttgtcgtgctggacagcctggcctgcacggatggtgccatcggtgcggcagcccagggctggctggtgcgtgcgctctccctcggggacgtggctcgcatcctcgaacccgtgctcctgctgctgctgcagccaaaaacccagagaacctccatccactgcctcaagcaggagaactcggccgatgacttgcaccgttggtttaacaggaagaaaacctctttcagagaggcatgcgcagtgcccgagcctcaggagagcggctctgaagagcacctgcctctgagccagttcaccacagtggaccgtgaagccatttgggccgaagtggagaaggagcccgagaagtacccgctgcgaggcgagctgagcgaggaagagctgccctactacgtggagcttccagacaggacggcccacggcgccccggacagcagcgagcacaccgagtctgcagatacaagctcctgccacacggacagcgagaacacgtcctccttctcctccccttcccacgacctgcaggagctgagcaacgaagagaactgctgtgcacccatccccatggggggcagggcgtaccccaagcgctcggccctgctggcggccttccagtcagaaagcttcaaggctggggccaagttaagcctggtgcgggtggactcggacaagacgcaggcttctgagtcgttctccagcgacgaggaggcggacttggagctccaggccctcaccacatccaggctgctaaagcagcagcgggaaaggcaggaggccgtcgaggccttgttcaagcacatcctgctctacctgcagccctacgactctcggcgggtcctctatgccttctcggtgctggaggctgtgctcaaaaccaaccctaaggaattcatcgaggctgtgtccaggactagcatggataccagctccaccgcgcacctcaacctcatctccaacctcctcgctcgccaccaggaggccctcattggccagagtttctacggaaagctccagacccaggtccccaacgtgtgcccccactctctgctcctggagctgctcacctacctctgcctgagcttcctgcgctcctactacccttgctatttgaaggtctcgcaccgagacattctcggcaaccgggacgtgcaggtcaaaagtgtcgaggttttgatcaggataatgatgcagctggtctcagtggccaagtcttcggaagggaagaacgtggagttcatccacagcttgctgcagaggtgcaaagttcaggagtttgtcctgctctccctgtcggcgtccatgtacacgagccagaagcgctacgggctggccaccgcccaccacggcagggccctgccagaggacagcctctttgaggagagtctcattaacttgggtcaggaccagatctggagtgagcacccgctgcagattgagctgctgaagctgctgcaggtgctgattgtcttggaacaccacctgggtcgggcccatgaggaggcggaaaaccagcccgacctgtcccgggagtggcagagagccctgaacttccagcaggccatcagcgccctgcagtacgtgcagccccaccccctcacctcccagggtcttctggtctctgcggtggtgaggggtctgcagcccgcctacggttacggcatgcatccggcctgggtgagcttggtcacgcattccttgccctacttcggaaagtccctgggctggacggtgacaccctttgttgtccagatttgcaaaaacttggatgacttggtcaagcagtatgaaagcgaatctgtgaagctctctgtcagcacaacctccaagagggaaaacatttctccagattatccactcacccttctagaaggtctaacgaccattagtcatttttgtcttttggaacaagccaaccaaaacaaaaagaccatggctgcaggtgatcctgccaacttgaggaatgccagaaatgccattttggaagagctgcctcgaactgttaacaccatggcccttctctggaatgttctcagaaaggaggagactcaaaagagacctgtcgatctcctaggggccacgaagggatcctcttccgtttactttaaaaccaccaaaaccataagacaaaaaattttagacttcttaaaccccttgacggcccatcttggggttcagttgacagcggctgttgcggcagtgtggagcagaaagaaagcccagcgtcacagtaagatgaagattatcccaacggcaagtgcatcccagctaacccttgtcgacttggtgtgtgcactcagcaccctgcagactgacacgctgctgcacctggtgaaggaggtggtgaagaggccaccccaagtcaaagggggtgatgagaaatcgcccctagtggacattcctgtgttgcagttttgctatgcttttctccaaaggctcccagtaccagccttgcaagagaacttttcttcactgttgggagtattgaaagagtctgtacagttgaatctagccccacctgggtattttctgcttctcagcatgctgaatgactttgtaacaagaactcccaacctggaaaacaagaaggaccaaaaagacctgcaggaaatcactcagaaaatcctagaagctgtggggaacattgccggctcttccttggagcaaaccagctggctaagcagaaacctggaagtgaaggcccaacctcaggcctctctagaagaatctgatgctgaggaggacctgtatgatgctgctgcagcttcagcaatggtgtcttcatccgccccgtcggtgtacagcgtgcaagccctctctctcctggcagaggtactggcttccctcctggacatggtttatcgaagtgatgagaaggagaaagctgtgccgttaatctcccgtctgctttactatgtttttccatacttacgcaaccacagtgcctacaatgctcccagcttccgggctggcgctcagctgctgagctccctgagtggctatgcctacacaaagcgagcctggaggaaggaggtcctggagctgtttctcgaccccgctttctttcagatggatacttcctgtgttcattggaagtccattattgaccatcttttgactcatgagaaaacaatgtttaaggatttaatgaacatgcagagcagttctttgaaactattctcaagttttgaacagaaagccatgctgttaaagcgccaggcttttgctgtcttcagtggagaacttgatcaataccacctttaccttccactgatacaagaacgcctgacagacaatctcagagttggacagacatccatagttgctgctcagatgtttctttttttcagagttttgctgctaagaatatctcctcaacatttgacttcattgtggccaataatggtctctgaattgattcagacattcacacagcttgaagaagatctaaaagatgaagatgagtcattgagaagcaccaacaaagtaaacagaacgaaagtttcagtcccggatgcaaatggaccctcagtgggggagataccccagagtgaactcatcttgtatttatcagcttgcaaattcttggacacagcgctttcttttccacctgacaagatgccattatttcaaatttataggtgggcatttattccagaagtggacacagagggccctgccttcctgtcggatgtagaggagaatcaccaagaatgcaaaccccacactgtcaggattctagaacttctaaaattaaagtttggggaaatcagtagctctgatgagatcaccatgaagagtgaattcccgcttctgcgccaacattctgtttccagcatcaggcagttgatgccattcttcatgactctaaatggtgcatttaagacccagagacagctgcctgctgatagcccaggaactccattcttggactttcctgtcacagatagcccaaggatcttaaaacaactggaagaatgcatcgaatatgattttctggaacatccagaatgttaaccatgtgagagagaatatgtttaatccatgtattggtactttactgaaaaccaggttatattctaaagaagaaagaaggcaggatagtgcttttgaacaagcctatttccattttgaaagtagatttcaggctaggtgcggtggctcacacctgtaatctcagcactttgggaggccaaggcaggcagatcacttgaggtcaggagttcgagaccagcctgaccaacatggtgagaccctgtctctactaaaaatacaaaaattagctgggtgtggtggcggcgcctgtaatcccagctacttgggaggctaaggcatgagaattgcttgaacccaggaggtggaggctgcagtgagccgagatcacgacactgcactccagctgtgtgacagaatgagaccatctccaaaaaaaaaaaaaagtagatttcagataatttactgttcagcaacaggacacacctccctaaatgccttgtaatatatttgaatctgattctgcatttcttcctcaatttatgtaatgaaaataaaattaatatatcatctaacagtagcacaaaatttgtaatatgaagtaaagtatgaagataatgaagaagttgttttctttgttgaagcagttatatgggtctttctcagtatatttctcttttctctaaaagtttaaacttattaaaagaatgttatttttaacctttcaaaaaaaaaaa

SEQ ID NO: 44 - DNA, 1138 bp, Homo sapiens
  source 1..1138: mol_type = other DNA
gctctggccggccccggcgattggtcaccgcccgctaggggacagccctggcctcctctgattggcaagcgctggccacctccccacaccccttgcgaacgctcccctagtggagaaaaggagtagctattagccaattcggcagggcccgctttttagaagcttgatttcctttgaagatgaaagactagcggaagctctgcctctttccccagtgggcgagggaactcggggcgattggctgggaactgtatccacccaaatgtcaccgatttcttcctatgcaggaaatgagcagacccatcaataagaaatttctcagcctggccgaaaatggttggccccacgaagccacgacaactggaggcaaagagggttgctcaacgccccgcctcattggaaaaccaaatcagatctgggacctatatagcgtggcggaggcggggcgatgattgtcgcgctcgcacccactgcagctgcgcacagtcgcatttctttccccgcccctgagaccctgcagcaccatctgtcatggcggctgggctgtttggtttgagcgctcgccgtcttttggcggcagcggcgacgcgagggctcccggccgcccgcgtccgctgggaatctagcttctccaggactgtggtcgccccgtccgctgtggcgggaaagcggcccccagaaccgaccacaccgtggcaagaggacccagaacccgaggacgaaaacttgtatgagaagaacccagactcccatggttatgacaaggaccccgttttggacgtctggaacatgcgacttgtcttcttctttggcgtctccatcatcctggtccttggcagcacctttgtggcctatctgcctgactacaggtgcacagggtgtccaagagcgtgggatgggatgaaagagtggtcccgccgcgaagctgagaggcttgtgaaataccgagaggccaatggccttcccatcatggaatccaactgcttcgaccccagcaagatccagctgccagaggatgagtgaccagttgctaagtggggctcaagaagcaccgccttccccaccccctgcctgccattctgacctcttctcagagcacctaattaaaggggctgaaagtctgaaaaaaaaaaaaaaa

SEQ ID NO: 45 - DNA, 1377 bp, Homo sapiens
  source 1..1377: mol_type = other DNA
atgctaaaactaatcgtcccaacaattatattactaccactgacatgactttccaaaaaacacataatttgaatcaacacaaccacccacagcctaattattagcatcatccctctactattttttaaccaaatcaacaacaacctatttagctgttccccaaccttttcctccgaccccctaacaacccccctcctaatactaactacctgactcctacccctcacaatcatggcaagccaacgccacttatccagtgaaccactatcacgaaaaaaactctacctctctatactaatctccctacaaatctccttaattataacattcacagccacagaactaatcatattttatatcttcttcgaaaccacacttatccccaccttggctatcatcacccgatgaggcaaccagccagaacgcctgaacgcaggcacatacttcctattctacaccctagtaggctcccttcccctactcatcgcactaatttacactcacaacaccctaggctcactaaacattctactactcactctcactgcccaagaactatcaaactcctgagccaacaacttaatatgactagcttacacaatagcttttatagtaaagatacctctttacggactccacttatgactccctaaagcccatgtcgaagcccccatcgctgggtcaatagtacttgccgcagtactcttaaaactaggcggctatggtataatacgcctcacactcattctcaaccccctgacaaaacacatagcctaccccttccttgtactatccctatgaggcataattataacaagctccatctgcctacgacaaacagacctaaaatcgctcattgcatactcttcaatcagccacatagccctcgtagtaacagccattctcatccaaaccccctgaagcttcaccggcgcagtcattctcataatcgcccacgggcttacatcctcattactattctgcctagcaaactcaaactacgaacgcactcacagtcgcatcataatcctctctcaaggacttcaaacttactcccactaatagctttttgatgacttctagcaagcctcgctaacctcgccttaccccccactattaacctactgggagaactctctgtgctagtaaccacgttctcctgatcaaatatcactctcctacttacaggactcaacatactagtcacagccctatactccctctacatatttaccacaacacaatggggctcactcacccaccacattaacaacataaaaccctcattcacacgagaaaacaccctcatgttcatacacctatcccccattctcctcctatccctcaaccccgacatcattaccgggttttcctctt

SEQ ID NO: 46 - DNA, 1779 bp, Homo sapiens
  source 1..1779: mol_type = other DNA
cggcgtgccctggggcggcgcgggcgcaggggcgcgtgcgcggcgggctgtcgttggctggagcagcggctgcgcgggtcgcggtgctgtgaggtctgcgggcgctggcaaatccggcccaggatgtagagctggcagtgcctgacggcgcgtctgacgcggagttgggtggggtagagagtagggggcggtagtcgggggtggtgggagaaggaggaggcggcgaatcacttataaatggcgccgaagcaggacccgaagcctaaattccaggaggttgggatgaatgggttccggagagcagagtactcaaatacgtggacaccaatttgcagaaacagcgagaacttcaaaaagccaatcaggagcagtatgcagaggggaagatgagaggggctgccccaggaaagaagacatctggtctgcaacagaaaaatgttgaagtgaaaacgaaaaagaacaaacagaaaacacctggaaatggagatggtggcagtaccagtgagacccctcagcctcctcggaagaaaagggcccgggtagatcctactgttgaaaatgaggaaacattcatgaacagagttgaagttaaagtaaagattcctgaagagctaaaaccgtggcttgttgatgactgggacttaattaccaggcaaaaacagctcttttatcttcctgccaagaagaatgtggattccattcttgaggattatgcaaattacaagaaatctcgtggaaacacagataataaggagtatgcggttaatgaagttgtggcagggataaaagaatacttcaacgtaatgttgggtacccagctactctataaatttgagagaccacagtatgctgaaattcttgcagatcatcccgatgcacccatgtcccaggtgtatggagcgccacatctcctgagattatttgtacgaattggagcaatgttggcttatacacctctggatgagaagagccttgctttattactcaattatcttcacgatttcctaaagtacctggcaaagaattctgcaactttgttcagtgccagcgattatgaagtggctcctcctgagtaccatcggaaagctgtgtgagaggcactctcactcacttatgtttggatctccgtaaacacatttttgttcttagtctatctcttgtacaaacgatgtgctttgaagatgttagtgtataacaattgatgtttgttttctgtttgattttaaacagagaaaaaataaaagggggtaatagctccttttttcttctttctttttttttttcatttcaaaattgctgccagtgttttcaatgatggacaacagagggatatgctgtagagtgttttattgcctagttgacaaagctgcttttgaatgctggtggttctattcctttgacactacgcacttttataatacatgttaatgctatatgacaaaatgctctgattcctagtgccaaaggttcaattcagtgtatataactgaacacactcatccatttgtgcttttgtttttttttatggtgcttaaagtaaagagcccatcctttgcaagtcatccatgttgttacttaggcattttatcttggctcaaattgttgaagaatggtggcttgtttcatggtttttgtatttgtgtctaatgcacgttttaacatgatagacgcaatgcattgtgtagctagttttctggaaaagtcaatcttttaggaattgtttttcagatcttcaataaattttttctttaaatttcaaagaacaaaaaaaaaaaaaaa

SEQ ID NO: 47 - DNA, 7827 bp, Homo sapiens
  source 1..7827: mol_type = other DNA
gtagtcttgacgtgagctagctggcatggcggcctgcattgcagcggggcactgggctgcaatgggcctaggccggagtttccaagccgccaggactctgctccccccgccggcctctatcgcctgcagggtccacgcggggcctgtccggcagcagagcactgggccttccgagcccggtgcgttccaaccgccgccgaaaccggtcatcgtggacaagcaccgccccgtggaaccggaacgcaggttcttgagtcctgaattcattcctcgaaggggaagaacagatcctctgaaatttcaaatagaaagaaaagatatgttagaaaggagaaaagtactccacattccagagttctatgttggaagtattcttcgtgttactacagctgacccatatgccagtggaaaaatcagccagtttctggggatttgcattcagagatcaggaagaggacttggagctactttcatccttaggaatgttatcgaaggacaaggtgtcgagatttgctttgaactttataatcctcgggtccaggagattcaggtggtcaaattagagaaacggctggatgatagcttgctatacttacgagatgcccttcctgaatatagcacttttgatgtgaatatgaagccagtagtacaagagcctaaccaaaaagttcctgttaatgagctgaaagtaaaaatgaagcctaagccctggtctaaacgctgggaacgtccaaattttaatattaaaggaatcagatttgatctttgtttaactgaacagcaaatgaaagaagctcagaagtggaatcagccatggcttgaatttgatatgatgagggaatatgatacttcaaaaattgaagctgcaatatggaaggaaattgaagcgtcgaaaaggtcttgattctgagaatgaatttggttagttgcagaagatacattggctctaagaggatatattttgagaccaatttaatttcatttataagaacatagtaattaagtgaactaagcattcattgttttattaatactttttttctaaaataaaacttgtacaccagtttattactctaaaaagagaattacacatgccaaatggaccaatgtccatttgcttattggaggcaaagctacaatagaagtcagagcatcaccagaatggtctttaatgagcatggaacctgagcaaagggaataggtgggatgaattttttttttaattgtgaaacaattcataagcacaatatgatttacagaataataaacattcatgtacccactatcaggttaagaaatagaacatttattaatatgtaggaatgttaagaaataaaacatttaataagatctcagaagactccagtaaatctgcaattgtatctctctcctttttaaatgtaaatatcatcttgacttgttaattattcccttgcatttcttttagtttactgccaacacatatattcttcaacaatatatttaattttgaaaaacctgaaaaaaaaaacctgttagcaagtataaaggggcagtattactattattgcatgaaggcttcaagggaaacgttacagtctttgggtcatagtctggcttcagcttcctctgagagtttacagaggccaattttgagcaaattcatggctaaggttatgagtgagttctgctaaacagaaggctcaccacaaggtatctggcaggattatactgggtagctggatgttgcagaaatgtggttagaggaagtaaactgttttttgatgctcacagcatgatgaatcaaactctgtatcttaggattaggttaaaacaatacctttggtatgatatgagtgttgttgctgatccatgcagcatggattggaaagctggggtataagcacacatgctaaagaaaaacatgtaatttggtccatactcacctggatatactgttcctcaggttaaaaaatacagtactatcctaaatcttgaaggcaactctcagcctatccattgagttaccttcagatctgccctctggttcctagctgtcttgggactaacttctttcctgcgctcagctgttttctggattccatgttttccattttattgagtactaacttgttttgctgcagcacatcctttggtagcttctagaggaagtttgtgtggaggtaaaatttttgagaccttgcatgtctcatgtttgattgatactttatacgtttaggtaggaggtaattttccttcaggactttaaaaatattgttgctccattttctttgtttctattgttgtattgagaaatccaatgccattttgatttccccatcataaatttcatgatgatgtgtcttggtgtgggtctatatttatccattgtattgggttttaggtgaacccttccagatagtaactcatttctgtcagttctgggaaacacttagcattggttgatgatttattctctgctgctttgttctcccaactattatttggatgttggatatccagcactgggtatctattttcttacctccctcccttgaccccagtctctgttttttagctctttagctcaatcttccaactctttgctattgtattttaaaatcttaagaccccttcttgatttgtagaagttccttttcttacaaccaaaaagcctttatctatggatttgttcacagataaggggtattcaatatagtgtatttttttttcatttaaaattgtttgcgcatctatttcctccaaatttctttctgtatttattttttgttgtctatatttcagacttttccaggatatctgataatctttggctgtcttcttatggttgaaagagggactaaaaagcttggaaagcctttgggttgtgggaaggggctgtctttaggattatctgaatgggcttttttgggagtcccctcctccacatgaatattttggttttgtcagattccctagaatagaggcttccaatctccttcctggaggggtctgtccaggaaggagattgtctaggggtctgtcagacagcagctttcagctacttccttgatctttttcactaatgattatatagtcatctaactactgtcaacaagtaatagatatcctatccttcacttgtttagattatttgctgagataacctctcaaaagaacctctcaaaataaaaggttaacaagagcctatatcttatatttttcttctctttatcttgttagaagatagctattaaaacctgttctttttctgtcttgataaacacacttcaatcttggtagaatggtagatgggacagtatattttaggacctaaagctctgcaaatgtatgatcagcttgtaagtacaggtgctcaaaaacatgtaaacaatcatgctttttactctgtaggaatatctttaaaattcttgtgaatttttccccagaagtaaagcaaatcttcccccagaaataaaattaaatgtgcataatctaaagctttttttttttattgtggtaggatatatatataaaacataatttgccattgtaaacattttaaatttacaagtcagaggcattaattacatcacaatgttgtgaaattattactactatttccaaaattttctcatcaccccaaactgaaactctgtaactgttgagcaataacctcattcctgtatctctcccaaccccaggtaacctcaaatctttctttttatctttgagacaaggtctcattctatcactcaggtaggagtgcagtggtgtgatcatagctcattgcagcctcaaaatcctgggctcaagcaatcctccttgagtagctaagactataggcacacattaactgcgcctggctgattttgttttttgtagagatgtggtcttgctatgtttcccatgctggtcttgagttcctggcctcaagcagtccttaagattcatccatgttgtggcatgtgtcagaatttcatttgtttttatgactaaataatattccattgtatgtatatacattttgttcatccatcttctgatgaacactgggatatgtctaccttttggctattgtgaataatgctgcagtaaacattgacataacaagtatgtatttgattgcctgtttctaagttcttttgggtatacatcttgagtagaattgctagataatgtcatgttttatttctcttgtgatttcttcttcgatcccctggttgagtgtgttaatttctacatgtttatgaatttcccactgtttttttgttattgatttccaagttcattccattgtgattagagaagatacttagtatgattttaatgtttttgagaattggtgtgtggcctgatagatggtctgtcctggagaatgttcctcatacacttgagcaaaatatttatcatgctattgttgactgtagttttctatatgtctcttaggtcaaggtggtttacaatgtgttaaggttctctttttttaaaaaaatttttgcacagagtatctttttctatgtgttccatgtatttgtgtctttggagctatagtctcttgtagacagcatatcactatcttgttttgttttgttttttctgtccattctgccaatttctgccttttgattggaaaatttaatccatttgcatttaaagtaattaaggaaggactttcttctaccatttaacacttcttctatatgtcatatacttttttggcccctcatttcctctttatggccttcttttctgtttttttgtagtgaactagtctgattctctttccactcccctttgtgtatatttgttagatgttttatttgtggttgctatggggattatagttaacatcctacacttaaaacaatctaatttaaactgataccaatttaccttcaatagcatacaaaatctctactcctgtaaagctctgcccctgccccccttatgttattgatggcacaaattgcctaataaataatttatagttatttgtatgagtttgtcttttaaatcatttaggaaataaaaagtggagttagaaaacagtatgatagtaatactgacttttatatttgtcaatatatttatcttattttggatccttatttcattatatagatttgagttactgtctagtgcccttccatttcggcccaaaggattcccttatgcatttcttgcagggcaagtctaattgtaataaactccctcagcttttgttttatctgagaatgtcttgatttctcccttatttttgatggataattttgccagatacatgaatttttggtaacagtatttttctttcagcactttaaatatgtcatcccactaccttctgacttcatggtttctcatgagatattagatgttataaaatttgaggattcctcattcttgatgagtcagttctgtcttattgcttttcggatttgctcagcttttgtcttttgacagtttgattataacgcggctcagtgtgggtctctgagtttatcccacttagagtttgttgagtttcttggagtcatagatttatgtcttttatcaaattttggacatatttggctattatttcttcaatttttttcactgcttctttcttttccttctgaaatattcttaatgtatatgttggtctgtttgatgctgtctcaccagtttcttaggctgtgttctcttttgttcctcagacttgattattgcagttgcccttctttttatttttttcaagtttgttgattcttctccctgttcagatcaactgttgaactcctctagtgaatttatttcagttactgtacttttcagctccaagatttatctttggttcctttttataacgtctgtgtctttattgatattctcattttgttcatatgtctctttcttcctttagttctttgtccatgttttcctttagctctttgggcttatttaagacaattgtttaaagtctttgcatagtaagtccaatgtctgtgtttcttcagggatggttttcattattttgttttcaatgagccatactttcctgtgtctttgtatgctgtctttttgttgttgaaaactgtatgtttgaacatcataacgtggtggccctgaaaatcagatattccccccttcctgagagttagttttatttttattattgaagattgtagcagtctattgctacatgtgcagtcatttccaaactatttttgcaaagactgtattccttctgtgtgtcatcactgaagtctctgttccttagtttgtgtttaatagtttgacatagatttccttgaaaggagttaaaactagcagaaaaatctctctcccagtctttccagtctttgtagattggttctgtgctgggcttttccattaatacttagccaggcttgtactgagcctaacaatcaggcccaaaagcgtagggtctttgcagatcttgtctgagcatgcttcttgctgtgtatgcacgtagttttctaaatctccctgtatgtgctgttgaatattctaatttcccaaagaaactcctttgcagctttttctcacagaacatagatggttttttggatatcttgaccatagtctttcgacccaggtgtttgcggttgttagttcaccttacacttttttcaagcattgcctactgcttacgatgagtgctctgtcaatcctttaagtagccccagacaggctaccagagacttaaacaagaatttgtaagttctgctcagcttcctctagaaatggggatcagggtccaagacagaatgcagttgctgatttcaagactgctgcaacaccagggagcttgtgggggaagggcaagcagaaatgtcacaaagctttcttgccattttaaagttgcctgttcttgactcagcatttgcttcattgctataaactttttactgtttttcagagttctgataaaattggctatgcctgttcctgctttaaaaaatatatatatattttttagggattggggtctcactatactgaccaggctggtcttgaacttctggcctcaagccatcctctcatttcagcttcccaaagtgctgcaattacacgcgtgaaccaccacacccagcccctgcttgtttttcaatgtgcctactccaccatgttgctcaagtatgtatattttctaaactaccttgtagtgttgtgatgggaaataaatccctgagccttttgaataactcagagagatcaaaaacttagtttatcctattcgaaggattagaaaaatgatatatctttcactttttcagggataggctcctcattagaaggctcctatgtgccgatgctgtacaagacatttcatttctcttaatgtttacaacaagcttgttgccaaggctgatcttgaactcctggcctcaaacgatcctcccagctcagtctcacaaagtgttgggatgtctggccaactaatgactatcttaactcttgtgtttcaatgtttatgccttcttttatcttgactgattgtatgactatgtcttctagaacaatgttgaacagaaatggtgagagcagacatccttgctttaatatttcaccattatatatgatgttaggtatagatttttctcacagatgccttttatcagattgaggaatttatattcctactttgccgaaaggtttttgtagtatgagggggtgctgaattttgtcaaacactttttcggtaataattgagatgattggttctgcagtcatcgagatgtggattttctcctttattctgttcgtgagtgattacactggttgactaatgttaaaacaaccttactttccaggaataaaccctattatcttttttataca

SEQ ID NO: 48 - DNA, 1821 bp, Homo sapiens
  source 1..1821: mol_type = other DNA
tgcgggtacggacagcgcatgagcttatgttgagggcggagcccagaccagcccttcgtcctatcctgcccttccagcacctctcagccgtaacttaaactacacttcccagaagcctcctcagccagggacttccgttgtcgtcagcggaagcggtgacagatcatcccaggccacacagaggccggcttggtcactatggaggagataggcatcttggtggagaaggctcaggatgagatcccagcactgtccgtgtcccggccccagaccggcctgtccttcctgggccctgagcctgaggacctggaggacctgtacagccgctacaaggaggaggtgaagcgaatccaaagcatcccgctggtcatcggacaatttctggaggctgtggatcagaatacagccatcgtgggctctaccacaggctccaactattatgtgcgcatcctgagcaccatcgatcgggagctgctcaagcccaacgcctcagtggccctccacaagcacagcaatgcactggtggacgtgctgccccccgaagccgacagcagcatcatgatgctcacctcagaccagaagccagatgtgatgtacgcggacatcggaggcatggacatccagaagcaggaggtgcgggaggccgtggagctcccgctcacgcatttcgagctctacaagcagatcggcatcgatcccccccgaggcgtcctcatgtatggcccacctggctgtgggaagaccatgttggcaaaggcggtggcacatcacacaacagctgcattcatccgggtcgtgggctcggagtttgtacagaagtatctgggtgagggcccccgcatggtccgggatgtgttccgcctggccaaggagaatgcacctgccatcatcttcatagacgagattgatgccatcgccaccaagagattcgatgctcagacaggggccgacagggaggttcagaggatcctgctggagctgctgaatcagatggatggatttgatcagaatgtcaatgtcaaggtaatcatggccacaaacagagcagacaccctggatccggccctgctacggccaggacggctggaccgtaaaattgaatttccacttcctgaccgccgccagaagagattgattttctccactatcactagcaagatgaacctctctgaggaggttgacttggaagactatgtggcccggccagataagatttcaggagctgatattaactccatctgtcaggagagtggaatgttggctgtccgtgaaaaccgctacattgtcctggccaaggacttcgagaaagcatacaagactgtcatcaagaaggacgagcaggagcatgagttttacaagtgacccttcccttccctccaccacaccactcaggggctggggcttctctcgcacccccagcacctctgtcccaaaacctcattcccttttttctttacccaggattggtttcttcaataaatagataagatcgaatccatttaatttcttcttagaagtttaactcctttggagaatgtgggccttgaataggatcctctgggtccctcttaatctgacagatgagcagacgaggtgcatggcctgggttgcagcttgagagaaccaaaatattcaaaccagatgacttccaaaatgtggggaaagggatggaaaatgaacctgagatggagtccttaatcacgggataaagccctgtgcatctccctcatttcctacaggtaaaagacagtaaagaaattcaggtcacaggccttgggagttcataggaaggagatgtccagtgctgtccagtagaacttt

SEQ ID NO: 49 - DNA, 5159 bp, Homo sapiens
  source 1..5159: mol_type = other DNA
ggtcccggaagtgcgccagtcgtaccttcgcggccgcaactcgctcggccgccgccatcttgcgagctcgtcgtactgaccgagcggggaggctgtcttgaggcggcaccgctcaccgacaccgaggcggactggcagccctgagcgtcgcagtcatgccggccggacccgtgcaggcggtgcccccgccgccgcccgtgcccacggagcccaaacagcccacagaagaagaagcatcttcaaaggaggattctgcaccttctaagccagttgtggggattatttaccctcctccagaggtcagaaatattgttgacaagactgccagctttgtggccagaaacgggcctgaatttgaagctaggatccgacagaacgagatcaacaaccccaagttcaactttctgaaccccaatgacccttaccatgcctactaccgccacaaggtcagcgagttcaaggaagggaaggctcaggagccgtccgccgccatccccaaggtcatgcagcagcagcagcagaccacccagcagcagctgccccagaaggtccaagcccaagtaatccaagagaccatcgtgcccaaagagcctcctcctgagtttgagttcattgctgatcctccctctatctcagccttcgacttggatgtggtgaagctgacggctcagtttgtggccaggaatgggcgccagtttctgacccagctgatgcagaaagagcagcgcaactaccagtttgactttctccgcccacagcacagcctcttcaactacttcacgaagctagtggaacagtacaccaagatcttgattccacccaaaggtttattttcaaagctcaagaaagaggctgaaaacccccgagaagttttggatcaggtgtgttaccgagtggaatgggccaaattccaggaacgtgagaggaagaaggaagaagaggagaaggagaaggagcgggtggcctatgctcagatcgactggcatgattttgtggtggtggaaacagtggacttccaacccaatgagcaagggaacttccctccccccaccacgccagaggagctgggggcccgaatcctcattcaggagcgctatgaaaagtttggggagagtgaggaagttgagatggaggtcgagtctgatgaggaggatgacaaacaggagaaggcggaggagcctccttcccagctggaccaggacacccaagtacaagatatggatgagggttcagatgatgaagaagaagggcagaaagtgcccccacccccagagacacccatgcctccacctctgcccccaactccagaccaagtcattgtccgcaaggattatgatcccaaagcctccaagcccttgcctccagcccctgctccagatgagtatcttgtgtcccccattactggggagaagatccccgccagcaaaatgcaggaacacatgcgcattggacttcttgaccctcgctggctggagcagcgggatcgctccatccgtgagaagcagagcgatgatgaggtgtacgcaccaggtctggatattgagagcagcttgaagcagttggctgagcggcgtactgacatcttcggtgtagaggaaacagccattggtaagaagatcggtgaggaggagatccagaagccagaggaaaaggtgacctgggatggccactcaggcagcatggcccggacccagcaggctgcccaggccaacatcaccctccaggagcagattgaggccattcacaaggccaaaggcctggtgccagaggatgacactaaagagaagattggccccagcaagcccaatgaaatccctcaacagccaccgccaccatcttcagccaccaacatccccagctcggctccacccatcacttcagtgccccgaccacccacaatgccacctccagttcgtactacagttgtctccgcagtacccgtcatgccccggcccccaatggcatctgtggtccggctgcccccaggctcagtgatcgcccccatgccgcccatcatccacgcgcccagaatcaacgtggtgcccatgcctccctcggcccctcctattatggccccccgcccaccccccatgattgtgccaacagcctttgtgcctgctccacctgtggcacctgtcccagctccagccccaatgccccctgtgcatcccccacctcccatggaagatgagcccacctccaaaaaactgaagacagaggacagcctcatgccagaggaggagttcctgcgcagaaacaagggtccagtgtccatcaaagtccaggtgcccaacatgcaggataagacggaatggaaactgaatgggcaggtgctggtcttcaccctcccactcacggaccaggtctctgtcattaaggtgaagattcatgaagccacaggcatgcctgcagggaaacagaagctacagtatgagggtatcttcatcaaagattccaactcactggcttactacaacatggccaatggcgcagtcatccacctggccctcaaggagagaggcgggaggaagaagtagacaagaggaacctgctgtcaagtccctgccattttgcctctcctgtctcccaccccctgccccagacccaggagcccccctgaggctttgccttgcctgcatatttgtttcgctcttactcagtttgggaattcaaattgtcctgcagaggttcattcccctgaccctttccccacattggtaagagtagctgggttttctaagccactctctggaatctctttgtgttagggtctcgatttgaggacattcatttcttcagcagcccattagcaactgagagcccagggatgtcctacaggatagtttcatagtgacaggtggcacttggctaatagaatatggctgatattgtcattaatcattttgtaccttgacatgggttgtctaataaaactcggacccttcttgtgaaatcagttaaataagacttgtctcggtcacctgtgccctgtccagactcgaggcagtggtaacactgcacagtgctatgtggcttctctttgaggatttttgggttttgtaactaaattcttgctgccctcatactttttatgtattagaatcatattcgtattgcccttttaaaacattgggatcctccaaaggcctgccccatgtatttaacagtaatacaggaagcatggcaggcaccatgcaaaccaaggatggatggtgcagtccctgtgtcagtgggcggtggtttcctgctggcctggaatcactcatcacctgattgattggctctgtggtcctgggcaggtgcctcataggtgtgtggatatgatgacgtttctttaaaatgtatgtatttaacaaatacttaattgtattaaggtcatgtaccaaggatttgataaagtttaaataatttactctctacttttatccattttatccattttaactcatgtaatcctcatgtgagtattcctgtttaacacttgagtaaactgaggcacagagaacataagttgcatgccatagtcacacactgtgaaagtgaaaagagaatgtgtgcaaaacacgtcacagtcctggtttctgagtaaaggcaggctgttatctttagaatcaagctatcacagggagataggcaatgctgtgggtgttggaggaaggtgagagcctgttgctaacaatttcctggttttaaagctaaggctgattttattgggaagatctcacatgtgtgtggcccctgagagttcccagtgccttttatttgcagtccttccatttggacctcctagctgccccatcaggtcatctccagggctcagaggggtgagaccatttcccaaggtcacagaaccagctctctagtcaccaccctgcctctccctctcacccagagtcagtaccagttttatggctttattacaaactgctgggtccctcccattttcaacttgattgatgggatgtcatcccttatcctgtctgacatttgcctctggcctggttgctagaagtttgccccaggggcaagagttgaaatttggcttcctgaggtgggctttgtggtttgcgtccctaaagtgagcccactactggttgcttgtccatggccaacaccagaaatcccctgagcactacctgggtctcattccaagaaggaagagggtcaggagacctggggagtctcatattccaagttcttctttctttctgggagcagtgggcagttcatggtgttagggcactcacccccacagactggcaaaccctgcaggacttccgtggctgaggctgtgaccggaggccaggaatgccgttgggtggattgtgagtgaatgggccctttgagctgccctctagagagcaaatccagtttcctggagctcctgaatgaatatctgtactggctcgctcagatgcagaagctccattgaccatgaggccttgtgaacatcagtggccacaggcccagtgtgctgcttggcactgcactagtttaggacctgcagcatgtaggtagcgtcctagtgtttataatacaaagctgctctgcacagcttttctgattcttcttgcaatctcctgaggattatctgccccatttttaaaacgaggtggaatacccaaggtcatgtagccagtgagtgctctggaaagccaaagcagctcatcccttcctggggaccacactgctctgctccaccagaccacactatgaaataggaataagtgctcctgttgcaggactgctgggaaaacaggtggtgtgggacttaagtcaccataattttgaagacttgcatgcagagggctccaggaattgtagacattaaggaatttcactttcagttctacccactacttaagtacttgtcatgtactcttagaggaggccagtaatgatcagaaccattttactttaaaattaataatattgtattagagaatatattaaatggttatattgggttatgttaggatatatacttgaatggaaatacatgtactattagcaatcatatttcatttatccctgtaattagacaagaaagcataatatagctctactcatgggtacacataccagtgtataagatttttagaagtttactttttaaaaataaaagcaaaatgtaagatcttaaaaaaaaaaaaaaaaaa

SEQ ID NO: 50 - DNA, 5416 bp, Homo sapiens
  source 1..5416: mol_type = other DNA
agtgggccgccatgttgtcggagtgaaaggtaagggggagcgagagcgccagagagagaagatcggggggctgaaatccatcttcatcctaccgctccgcccgtgttggtggaatgagcgttgcatgtgtcttgaagagaaaagcagtgctttggcaggactctttcagcccccacctgaaacatcaccctcaagaaccagctaatcccaacatgcctgttgttttgacatctggaacagggtcgcaagcgcagccacaaccagctgcaaatcaggctcttgcagctgggactcactccagccctgtcccaggatctataggagttgcaggccgttcccaggacgacgctatggtggactacttctttcagaggcagcatggtgagcagcttgggggaggaggaagtggaggaggcggctataataatagcaaacatcgatggcctactggggataacattcatgcagaacatcaggtgcgttccatggatgaactgaatcatgattttcaagcacttgctctggagggaagagcgatgggagagcagctcttgccaggtaaaaagttttgggaaacagatgaatccagcaaagatggaccaaaaggaatattcctgggtgatcaatggcgagacagtgcctggggaacatcagatcattcagtttcccagccaatcatggtgcagagaagacctggtcagagtttccatgtgaacagtgaggtcaattctgtactgtccccacgatcggagagtgggggactaggcgttagcatggtggagtatgtgttgagctcatccccgggcgattcctgtctaagaaaaggaggatttggcccaagggatgcagacagtgatgaaaacgacaaaggtgaaaagaagaacaagggtacgtttgatggagataagctaggagatttgaaggaggagggtgatgtgatggacaagaccaatggtttaccagtgcagaatgggattgatgcagacgtcaaagattttagccgtacccctggtaattgccagaactctgctaatgaagtggatcttctgggtccaaaccagaatggttctgagggcttagcccagctgaccagcaccaatggtgccaagcctgtggaggatttctccaacatggagtcccagagtgtccccttggaccccatggaacatgtgggcatggagcctcttcagtttgattattcaggcacgcaggtacctgtggactcagcagcagcaactgtgggactttttgactacaattctcaacaacagctgttccaaagacctaatgcgcttgctgtccagcagttgacagctgctcagcagcagcagtatgcactggcagctgctcatcagccgcacatcggtttagctcccgctgcgtttgtccccaatccatacatcatcagcgctgctcccccagggacggacccctacacagctggattggctgcagcagcgacactaggcccagctgtggtccctcaccagtattatggagttactccctggggagtctaccctgccagtcttttccagcagcaagctgccgctgccgctgcagcaactaattcagctaatcaacagaccaccccacaggctcagcaaggacagcagcaggttctccgtggaggagccagccaacgtcctttgaccccaaaccagaaccagcagggacagcaaacggatccccttgtggcagctgcagcagtgaattctgcccttgcatttggacaaggtctggcagcaggcatgccaggttatccggtgttggctcctgctgcttactatgaccaaactggtgcccttgtagtgaatgcaggcgcgagaaatggtcttggagctcctgttcgacttgtagctcctgccccagtcatcattagttcctcagctgcacaagcagctgttgcagcagccgcagcttcagcaaatggagcagctggtggtcttgctggaacaacaaatggaccatttcgccctttaggaacacagcagcctcagccccagccccagcagcagcccaataacaacctggcatccagttctttctacggcaacaactctctgaacagcaattcacagagcagctccctcttctcccagggctctgcccagcctgccaacacatccttgggattcggaagtagcagttctctcggcgccaccctgggatccgcccttggagggtttggaacagcagttgcaaactccaacactggcagtggctcccgccgtgactccctgactggcagcagtgacctttataagaggacatcgagcagcttgacccccattggacacagtttttataacggccttagcttttcctcctctcctggacccgtgggcatgcctctccctagtcagggaccaggacattcacagacaccacctccttccctctcttcacatggatcctcttcaagcttaaacctgggaggactcacgaatggcagtggaagatacatctctgctgctccaggcgctgaagccaagtaccgcagtgcaagcagcgcctccagcctcttcagcccgagcagcactcttttctcttcctctcgtttgcgatatggaatgtctgatgtcatgccttctggcaggagcaggcttttggaagattttcgaaacaaccggtaccccaatttacaactgcgggagattgctggacatataatggaattttcccaagaccagcatgggtccagattcattcagctgaaactggagcgtgccacaccagctgagcgccagcttgtcttcaatgaaatcctccaggctgcctaccaactcatggtggatgtgtttggtaattacgtcattcagaagttctttgaatttggcagtcttgaacagaagctggctttggcagaacggattcgaggccacgtcctgtcattggcactacagatgtatggctgccgtgttatccagaaagctcttgagtttattccttcagaccagcaggtaattaatgagatggttcgggaactagatggccatgtcttgaagtgtgtgaaagatcagaatggcaatcacgtggttcagaaatgcattgaatgtgtacagccccagtctttgcaatttatcatcgatgcgtttaagggacaggtatttgccttatccacacatccttatggctgccgagtgattcagagaatcctggagcactgtctccctgaccagacactccctattttagaggagcttcaccagcacacagagcagcttgtacaggatcaatatggaaattatgtaatccaacatgtactggagcacggtcgtcctgaggataaaagcaaaattgtagcagaaatccgaggcaatgtacttgtattgagtcagcacaaatttgcaagcaatgttgtggagaagtgtgttactcacgcctcacgtacggagcgcgctgtgctcatcgatgaggtgtgcaccatgaacgacggtccccacagtgccttatacaccatgatgaaggaccagtatgccaactacgtggtccagaagatgattgacgtggcggagccaggccagcggaagatcgtcatgcataagatccggccccacatcgcaactcttcgtaagtacacctatggcaagcacattctggccaagctggagaagtactacatgaagaacggtgttgacttagggcccatctgtggcccccctaatggtatcatctgaggcagtgtcacccgctgttccctcattcccgctgacctcactggcccactggcaaatccaaccagcaaccagaaatgttctagtgtagagtctgagacgggcaagtggttgctccaggattactccctcctccaaaaaaggaatcaaatccacgagtggaaaagcctttgtaaatttaattttattacacataacatgtactattttttttaattgactaattgccctgctgttttactggtgtataggatacttgtacataggtaaccaatgtacatgggaggccacatattttgttcactgttgtatctatatttcacatgtggaaactttcagggtggttggtttaacaaaaaaaaaaagctttaaaaaaaaaagaaaaaaaggaaaaggtttttagctcatttgcctggccggcaagttttgcaaatagctcttccccacctcctcattttagtaaaaaacaaacaaaaacaaaaaaacctgagaagtttgaattgtagttaaatgaccccaaactggcatttaacactgtttataaaaaatatatatatatatatatatatatataatgaaaaaggtttcagagttgctaaagcttcagtttgtgacattaagtttatgaaattctaaaaaatgccttttttggagactatattatgctgaagaaggctgttcgtgaggaggagatgcgagcacccagaacgtcttttgaggctgggcgggtgtgattgtttactgcctactggatttttttctattaacattgaaaggtaaaatctgattatttagcatgagaaaaaaaaatccaactctgcttttggtcttgcttctataaatatatagtgtatacttggtgtagactttgcatatatacaaatttgtagtattttcttgttttgatgtctaatctgtatctataatgtaccctagtagtcgaacatacttttgattgtacaattgtacatttgtatacctgtaatgtaaatgtggagaagtttgaatcaacataaacacgttttttggtaagaaaagagaattagccagccctgtgcattcagtgtatattctcaccttttatggtcgtagcatatagtgttgtatattgtaaattgtaatttcaaccagaagtaaatttttttcttttgaaggaataaatgttctttatacagcctagttaatgtttaaaaagaaaaaaatagcttggttttatttgtcatctagtctcaagtatagcgagattctttctaaatgttattcaagattgagttctcactagtgtttttttaatcctaaaaaagtaatgttttgattttgtgacagtcaaaaggacgtgcaaaagtctagccttgcccgagctttccttacaatcagagcccctctcaccttgtaaagtgtgaatcgcccttcccttttgtacagaagatgaactgtattttgcattttgtctacttgtaagtgaatgtaacatactgtcaattttccttgtttgaatatagaattgtaacactacacggtgtacatttccagagccttgtgtatatttccaatgaacttttttgcaagcacacttgtaaccatatgtgtataattaacaaacctgtgtatgcttatgcctgggcaactattttttgtaactcttgtgtagattgtctctaaacaatgtgtgatctttattttgaaaaatacagaactttggaatctgaaaaaaaaaaaaaaaaaaaaaaaaaaaaaaa

SEQ ID NO: 51 - DNA, 1940 bp, Homo sapiens
  source 1..1940: mol_type = other DNA
gagtgagcggcgcggggccaatcagcgtgcgccgttccgaaagttgccttttatggctcgagcggccgcggcggcgccctataaaacccagcggcgcgacgcgccaccaccgccgagaccgcgtccgccccgcgagcacagagcctcgcctttgccgatccgccgcccgtccacacccgccgccagctcaccatggatgatgatatcgccgcgctcgtcgtcgacaacggctccggcatgtgcaaggccggcttcgcgggcgacgatgccccccgggccgtcttcccctccatcgtggggcgccccaggcaccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctccctggagaagagctacgagctgcctgacggccaggtcatcaccattggcaatgagcggttccgctgccctgaggcactcttccagccttccttcctgggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaagatcattgctcctcctgagcgcaagtactccgtgtggatcggcggctccatcctggcctcgctgtccaccttccagcagatgtggatcagcaagcaggagtatgacgagtccggcccctccatcgtccaccgcaaatgcttctaggcggactatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcgttgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttcccccttttttgtcccccaacttgagatgtatgaaggcttttggtctccctgggagtgggtggaggcagccagggcttacctgtacactgacttgagaccagttgaataaaagtgcacaccttaaaaatgaggaaaaaaaaaaaaaaaaaa

SEQ ID NO: 52 - DNA, 1309 bp, Homo sapiens
  source 1..1309: mol_type = other DNA
gctctctgctcctcctgttcgacagtcagccgcatcttcttttgcgtcgccagccgagccacatcgctcagacaccatggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacagcctcaagatcatcagcaatgcctcctgcaccaccaactgcttagcacccctggccaaggtcatccatgacaactttggtatcgtggaaggactcatgaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgacaacgaatttggctacagcaacagggtggtggacctcatggcccacatggcctccaaggagtaagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaaccagttaaaaaaaaaaaaaaaaaaaaa

SEQ ID NO: 53 - DNA, 2321 bp, Homo sapiens
  source 1..2321: mol_type = other DNA
gtcctcaaccaagatggcgcggatggcttcaggcgcatcacgacaccggcgcgtcacgcgacccgccctacgggcacctcccgcgcttttcttagcgccgcagacggtggccgagcgggggaccgggaagcatggcccgggggtcggcggttgcctgggcggcgctcgggccgttgttgtggggctgcgcgctggggctgcagggcgggatgctgtacccccaggagagcccgtcgcgggagtgcaaggagctggacggcctctggagcttccgcgccgacttctctgacaaccgacgccggggcttcgaggagcagtggtaccggcggccgctgtgggagtcaggccccaccgtggacatgccagttccctccagcttcaatgacatcagccaggactggcgtctgcggcattttgtcggctgggtgtggtacgaacgggaggtgatcctgccggagcgatggacccaggacctgcgcacaagagtggtgctgaggattggcagtgcccattcctatgccatcgtgtgggtgaatggggtcgacacgctagagcatgaggggggctacctccccttcgaggccgacatcagcaacctggtccaggtggggcccctgccctcccggctccgaatcactatcgccatcaacaacacactcacccccaccaccctgccaccagggaccatccaatacctgactgacacctccaagtatcccaagggttactttgtccagaacacatattttgactttttcaactacgctggactgcagcggtctgtacttctgtacacgacacccaccacctacatcgatgacatcaccgtcaccaccagcgtggagcaagacagtgggctggtgaattaccagatctctgtcaagggcagtaacctgttcaagttggaagtgcgtcttttggatgcagaaaacaaagtcgtggcgaatgggactgggacccagggccaacttaaggtgccaggtgtcagcctctggtggccgtacctgatgcacgaacgccctgcctatctgtattcattggaggtgcagctgactgcacagacgtcactggggcctgtgtctgacttctacacactccctgtggggatccgcactgtggctgtcaccaagagccagttcctcatcaatgggaaacctttctatttccacggtgtcaacaagcatgaggatgcggacatccgagggaagggcttcgactggccgctgctggtgaaggacttcaacctgcttcgctggcttggtgccaacgctttccgtaccagccactacccctatgcagaggaagtgatgcagatgtgtgaccgctatgggattgtggtcatcgatgagtgtcccggcgtgggcctggcgctgccgcagttcttcaacaacgtttctctgcatcaccacatgcaggtgatggaagaagtggtgcgtagggacaagaaccaccccgcggtcgtgatgtggtctgtggccaacgagcctgcgtcccacctagaatctgctggctactacttgaagatggtgatcgctcacaccaaatccttggacccctcccggcctgtgacctttgtgagcaactctaactatgcagcagacaagggggctccgtatgtggatgtgatctgtttgaacagctactactcttggtatcacgactacgggcacctggagttgattcagctgcagctggccacccagtttgagaactggtataagaagtatcagaagcccattattcagagcgagtatggagcagaaacgattgcagggtttcaccaggatccacctctgatgttcactgaagagtaccagaaaagtctgctagagcagtaccatctgggtctggatcaaaaacgcagaaaatacgtggttggagagctcatttggaattttgccgatttcatgactgaacagtcaccgacgagagtgctggggaataaaaaggggatcttcactcggcagagacaaccaaaaagtgcagcgttccttttgcgagagagatactggaagattgccaatgaaaccaggtatccccactcagtagccaagtcacaatgtttggaaaacagcctgtttacttgagcaagactgataccacctgcgtgtcccttcctccccgagtcagggcgacttccacagcagcagaacaagtgcctcctggactgttcacggcagaccagaacgtttctggcctgggttttgtggtcatctattctagcagggaacactaaaggtggaaataaaagattttctattatggaaataaagagttggcatgaaagtggctactgaaaaaaaaaaaaaaaaaaaaaaaaa

SEQ ID NO: 54 - DNA, 1229 bp, Homo sapiens
  source 1..1229: mol_type = other DNA
gtctgacgggcgatggcgcagccaatagacaggagcgctatccgcggtttctgattggctactttgttcgcattataaaaggcacgcgcgggcgcgaggcccttctctcgccaggcgtcctcgtggaagtgacatcgtctttaaaccctgcgtggcaatccctgacgcaccgccgtgatgcccagggaagacagggcgacctggaagtccaactacttccttaagatcatccaactattggatgattatccgaaatgtttcattgtgggagcagacaatgtgggctccaagcagatgcagcagatccgcatgtcccttcgcgggaaggctgtggtgctgatgggcaagaacaccatgatgcgcaaggccatccgagggcacctggaaaacaacccagctctggagaaactgctgcctcatatccgggggaatgtgggctttgtgttcaccaaggaggacctcactgagatcagggacatgttgctggccaataaggtgccagctgctgcccgtgctggtgccattgccccatgtgaagtcactgtgccagcccagaacactggtctcgggcccgagaagacctcctttttccaggctttaggtatcaccactaaaatctccaggggcaccattgaaatcctgagtgatgtgcagctgatcaagactggagacaaagtgggagccagcgaagccacgctgctgaacatgctcaacatctcccccttctcctttgggctggtcatccagcaggtgttcgacaatggcagcatctacaaccctgaagtgcttgatatcacagaggaaactctgcattctcgcttcctggagggtgtccgcaatgttgccagtgtctgtctgcagattggctacccaactgttgcatcagtaccccattctatcatcaacgggtacaaacgagtcctggccttgtctgtggagacggattacaccttcccacttgctgaaaaggtcaaggccttcttggctgatccatctgcctttgtggctgctgcccctgtggctgctgccaccacagctgctcctgctgctgctgcagccccagctaaggttgaagccaaggaagagtcggaggagtcggacgaggatatgggatttggtctctttgactaatcaccaaaaagcaaccaacttagccagttttatttgcaaaacaaggaaataaaggcttacttctttaaaaagtaaaaaaaaaaaaaaaaaaaaaaaaa

SEQ ID NO: 55 - DNA, 5234 bp, Homo sapiens
  source 1..5234: mol_type = other DNA
agagcgtcgggatatcgggtggcggctcgggacggaggacgcgctagtgtgagtgcgggcttctagaactacaccgaccctcgtgtcctcccttcatcctgcggggctggctggagcggccgctccggtgctgtccagcagccatagggagccgcacggggagcgggaaagcggtcgcggccccaggcggggcggccgggatggagcggggccgcgagcctgtggggaaggggctgtggcggcgcctcgagcggctgcaggttcttctgtgtggcagttcagaatgatggatcaagctagatcagcattctctaacttgtttggtggagaaccattgtcatatacccggttcagcctggctcggcaagtagatggcgataacagtcatgtggagatgaaacttgctgtagatgaagaagaaaatgctgacaataacacaaaggccaatgtcacaaaaccaaaaaggtgtagtggaagtatctgctatgggactattgctgtgatcgtctttttcttgattggatttatgattggctacttgggctattgtaaaggggtagaaccaaaaactgagtgtgagagactggcaggaaccgagtctccagtgagggaggagccaggagaggacttccctgcagcacgtcgcttatattgggatgacctgaagagaaagttgtcggagaaactggacagcacagacttcaccggcaccatcaagctgctgaatgaaaattcatatgtccctcgtgaggctggatctcaaaaagatgaaaatcttgcgttgtatgttgaaaatcaatttcgtgaatttaaactcagcaaagtctggcgtgatcaacattttgttaagattcaggtcaaagacagcgctcaaaactcggtgatcatagttgataagaacggtagacttgtttacctggtggagaatcctgggggttatgtggcgtatagtaaggctgcaacagttactggtaaactggtccatgctaattttggtactaaaaaagattttgaggatttatacactcctgtgaatggatctatagtgattgtcagagcagggaaaatcacctttgcagaaaaggttgcaaatgctgaaagcttaaatgcaattggtgtgttgatatacatggaccagactaaatttcccattgttaacgcagaactttcattctttggacatgctcatctggggacaggtgacccttacacacctggattcccttccttcaatcacactcagtttccaccatctcggtcatcaggattgcctaatatacctgtccagacaatctccagagctgctgcagaaaagctgtttgggaatatggaaggagactgtccctctgactggaaaacagactctacatgtaggatggtaacctcagaaagcaagaatgtgaagctcactgtgagcaatgtgctgaaagagataaaaattcttaacatctttggagttattaaaggctttgtagaaccagatcactatgttgtagttggggcccagagagatgcatggggccctggagctgcaaaatccggtgtaggcacagctctcctattgaaacttgcccagatgttctcagatatggtcttaaaagatgggtttcagcccagcagaagcattatctttgccagttggagtgctggagactttggatcggttggtgccactgaatggctagagggatacctttcgtccctgcatttaaaggctttcacttatattaatctggataaagcggttcttggtaccagcaacttcaaggtttctgccagcccactgttgtatacgcttattgagaaaacaatgcaaaatgtgaagcatccggttactgggcaatttctatatcaggacagcaactgggccagcaaagttgagaaactcactttagacaatgctgctttccctttccttgcatattctggaatcccagcagtttctttctgtttttgcgaggacacagattatccttatttgggtaccaccatggacacctataaggaactgattgagaggattcctgagttgaacaaagtggcacgagcagctgcagaggtcgctggtcagttcgtgattaaactaacccatgatgttgaattgaacctggactatgagaggtacaacagccaactgctttcatttgtgagggatctgaaccaatacagagcagacataaaggaaatgggcctgagtttacagtggctgtattctgctcgtggagacttcttccgtgctacttccagactaacaacagatttcgggaatgctgagaaaacagacagatttgtcatgaagaaactcaatgatcgtgtcatgagagtggagtatcacttcctctctccctacgtatctccaaaagagtctcctttccgacatgtcttctggggctccggctctcacacgctgccagctttactggagaacttgaaactgcgtaaacaaaataacggtgcttttaatgaaacgctgttcagaaaccagttggctctagctacttggactattcagggagctgcaaatgccctctctggtgacgtttgggacattgacaatgagttttaaatgtgatacccatagcttccatgagaacagcagggtagtctggtttctagacttgtgctgatcgtgctaaattttcagtagggctacaaaacctgatgttaaaattccatcccatcatcttggtactactagatgtctttaggcagcagcttttaatacagggtagataacctgtacttcaagttaaagtgaataaccacttaaaaaatgtccatgatggaatattcccctatctctagaattttaagtgctttgtaatgggaactgcctctttcctgttgttgttaatgaaaatgtcagaaaccagttatgtgaatgatctctctgaatcctaagggctggtctctgctgaaggttgtaagtggtcgcttactttgagtgatcctccaacttcatttgatgctaaataggagataccaggttgaaagaccttctccaaatgagatctaagcctttccataaggaatgtagctggtttcctcattcctgaaagaaacagttaactttcagaagagatgggcttgttttcttgccaatgaggtctgaaatggaggtccttctgctggataaaatgaggttcaactgttgattgcaggaataaggccttaatatgttaacctcagtgtcatttatgaaaagaggggaccagaagccaaagacttagtatattttcttttcctctgtcccttcccccataagcctccatttagttctttgttatttttgtttcttccaaagcacattgaaagagaaccagtttcaggtgtttagttgcagactcagtttgtcagactttaaagaataatatgctgccaaattttggccaaagtgttaatcttaggggagagctttctgtccttttggcactgagatatttattgtttatttatcagtgacagagttcactataaatggtgtttttttaatagaatataattatcggaagcagtgccttccataattatgacagttatactgtcggttttttttaaataaaagcagcatctgctaataaaacccaacagatactggaagttttgcatttatggtcaacacttaagggttttagaaaacagccgtcagccaaatgtaattgaataaagttgaagctaagatttagagatgaattaaatttaattaggggttgctaagaagcgagcactgaccagataagaatgctggttttcctaaatgcagtgaattgtgaccaagttataaatcaatgtcacttaaaggctgtggtagtactcctgcaaaattttatagctcagtttatccaaggtgtaactctaattcccattttgcaaaatttccagtacctttgtcacaatcctaacacattatcgggagcagtgtcttccataatgtataaagaacaaggtagtttttacctaccacagtgtctgtatcggagacagtgatctccatatgttacactaagggtgtaagtaattatcgggaacagtgtttcccataattttcttcatgcaatgacatcttcaaagcttgaagatcgttagtatctaacatgtatcccaactcctataattccctatcttttagttttagttgcagaaacattttgtggtcattaagcattgggtgggtaaattcaaccactgtaaaatgaaattactacaaaatttgaaatttagcttgggtttttgttacctttatggtttctccaggtcctctacttaatgagatagtagcatacatttataatgtttgctattgacaagtcattttaactttatcacattatttgcatgttacctcctataaacttagtgcggacaagttttaatccagaattgaccttttgacttaaagcagagggactttgtatagaaggtttgggggctgtggggaaggagagtcccctgaaggtctgacacgtctgcctacccattcgtggtgatcaattaaatgtaggtatgaataagttcgaagctccgtgagtgaaccatcattataaacgtgatgatcagctgtttgtcatagggcagttggaaacggcctcctagggaaaagttcatagggtctcttcaggttcttagtgtcacttacctagatttacagcctcacttgaatgtgtcactactcacagtctctttaatcttcagttttatctttaatctcctcttttatcttggactgacatttagcgtagctaagtgaaaaggtcatagctgagattcctggttcgggtgttacgcacacgtacttaaatgaaagcatgtggcatgttcatcgtataacacaatatgaatacagggcatgcattttgcagcagtgagtctcttcagaaaacccttttctacagttagggttgagttacttcctatcaagccagtacgtgctaacaggctcaatattcctgaatgaaatatcagactagtgacaagctcctggtcttgagatgtcttctcgttaaggagatgggccttttggaggtaaaggataaaatgaatgagttctgtcatgattcactattctagaacttgcatgacctttactgtgttagctctttgaatgttcttgaaattttagactttctttgtaaacaaatgatatgtccttatcattgtataaaagctgttatgtgcaacagtgtggagattccttgtctgatttaataaaatacttaaacactgaaaaaaaaaaa

SEQ ID NO: 56 - DNA, 1869 bp, Homo sapiens
  source 1..1869: mol_type = other DNA
tacctggttgatcctgccagtagcatatgcttgtctcaaagattaagccatgcatgtctaagtacgcacggccggtacagtgaaactgcgaatggctcattaaatcagttatggttcctttggtcgctcgctcctctcccacttggataactgtggtaattctagagctaatacatgccgacgggcgctgacccccttcgcgggggggatgcgtgcatttatcagatcaaaaccaacccggtcagcccctctccggccccggccggggggcgggcgccggcggctttggtgactctagataacctcgggccgatcgcacgccccccgtggcggcgacgacccattcgaacgtctgccctatcaactttcgatggtagtcgccgtgcctaccatggtgaccacgggtgacggggaatcagggttcgattccggagagggagcctgagaaacggctaccacatccaaggaaggcagcaggcgcgcaaattacccactcccgacccggggaggtagtgacgaaaaataacaatacaggactctttcgaggccctgtaattggaatgagtccactttaaatcctttaacgaggatccattggagggcaagtctggtgccagcagccgcggtaattccagctccaatagcgtatattaaagttgctgcagttaaaaagctcgtagttggatcttgggagcgggcgggcggtccgccgcgaggcgagccaccgcccgtccccgccccttgcctctcggcgccccctcgatgctcttagctgagtgtcccgcggggcccgaagcgtttactttgaaaaaattagagtgttcaaagcaggcccgagccgcctggataccgcagctaggaataatggaataggaccgcggttctattttgttggttttcggaactgaggccatgattaagagggacggccgggggcattcgtattgcgccgctagaggtgaaattcttggaccggcgcaagacggaccagagcgaaagcatttgccaagaatgttttcattaatcaagaacgaaagtcggaggttcgaagacgatcagataccgtcgtagttccgaccataaacgatgccgaccggcgatgcggcggcgttattcccatgacccgccgggcagcttccgggaaaccaaagtctttgggttccggggggagtatggttgcaaagctgaaacttaaaggaattgacggaagggcaccaccaggagtggagcctgcggcttaatttgactcaacacgggaaacctcacccggcccggacacggacaggattgacagattgatagctctttctcgattccgtgggtggtggtgcatggccgttcttagttggtggagcgatttgtctggttaattccgataacgaacgagactctggcatgctaactagttacgcgacccccgagcggtcggcgtcccccaacttcttagagggacaagtggcgttcagccacccgagattgagcaataacaggtctgtgatgcccttagatgtccggggctgcacgcgcgctacactgactggctcagcgtgtgcctaccctacgccggcaggcgcgggtaacccgttgaaccccattcgtgatggggatcggggattgcaattattccccatgaacgaggaattcccagtaagtgcgggtcataagcttgcgttgattaagtccctgccctttgtacacaccgcccgtcgctactaccgattggatggtttagtgaggccctcggatcggccccgccggggtcggcccacggccctggcggagcgctgagaagacggtcgaacttgactatctagaggaagtaaaagtcgtaacaaggtttccgtaggtgaacctgcggaaggatcatta

SEQ ID NO: 57 - DNA, 2288 bp, Homo sapiens
  source 1..2288: mol_type = other DNA
ggggccgaacgtggtataaaaggggcgggaggccaggctcgtgccgttttgcagacgccaccgccgaggaaaaccgtgtactattagccatggtcaaccccaccgtgttcttcgacattgccgtcgacggcgagcccttgggccgcgtctcctttgagctgtttgcagacaaggtcccaaagacagcagaaaattttcgtgctctgagcactggagagaaaggatttggttataagggttcctgctttcacagaattattccagggtttatgtgtcagggtggtgacttcacacgccataatggcactggtggcaagtccatctatggggagaaatttgaagatgagaacttcatcctaaagcatacgggtcctggcatcttgtccatggcaaatgctggacccaacacaaatggttcccagtttttcatctgcactgccaagactgagtggttggatggcaagcatgtggtgtttggcaaagtgaaagaaggcatgaatattgtggaggccatggagcgctttgggtccaggaatggcaagaccagcaagaagatcaccattgctgactgtggacaactcgaataagtttgacttgtgttttatcttaaccaccagatcattccttctgtagctcaggagagcacccctccaccccatttgctcgcagtatcctagaatctttgtgctctcgctgcagttccctttgggttccatgttttccttgttccctcccatgcctagctggattgcagagttaagtttatgattatgaaataaaaactaaataacaattgtcctcgtttgagttaagagtgttgatgtaggctttattttaagcagtaatgggttacttctgaaacatcacttgtttgcttaattctacacagtacttagattttttttactttccagtcccaggaagtgtcaatgtttgttgagtggaatattgaaaatgtaggcagcaactgggcatggtggctcactgtctgtaatgtattacctgaggcagaagaccacctgagggtaggagtcaagatcagcctgggcaacatagtgagacgctgtctctacaaaaaataattagcctggcctggtggtgcatgcctagtcctagctgatctggaggctgacgtgggaggattgcttgagcctagagtgagctattatcatgccactgtacagcctgggtgttcacagatcttgtgtctcaaaggtaggcagaggcaggaaaagcaaggagccagaattaagaggttgggtcagtctgcagtgagttcatgcatttagaggtgttcttcaagatgactaatgtcaaaaattgagacatctgttgcggttttttttttttttttttcccctggaatgcagtggcgtgatctcagctcactgcagcctccgcctcctgggttcaagtgattctagtgcctcagcctcctgagtagctgggataatgggcgtgtgccaccatgcccagctaatttttgtatttttagtatagatggggtttcatcattttgaccaggctggtctcaaactcttgacctcagctgatgcgcctgccttggcctcccaaactgctgagattacagatgtgagccaccgcaccctacctcattttctgtaacaaagctaagcttgaacactgttgatgttcttgagggaagcatattgggctttaggctgtaggtcaagtttatacatcttaattatggtggaattcctatgtagagtctaaaaagccaggtacttggtgctacagtcagtctccctgcagagggttaaggcgcagactacctgcagtgaggaggtactgcttgtagcatatagagcctctccctagctttggttatggaggctttgaggttttgcaaacctgaccaatttaagccataagatctggtcaaagggatacccttcccactaaggacttggtttctcaggaaattatatgtacagtgcttgctggcagttagatgtcaggacaatctaagctgagaaaaccccttctctgcccaccttaacagacctctagggttcttaacccagcaatcaagtttgcctatcctagaggtggcggatttgatcatttggtgtgttgggcaatttttgttttactgtctggttccttctgcgtgaattaccaccaccaccacttgtgcatctcagtcttgtgtgttgtctggttacgtattccctgggtgataccattcaatgtcttaatgtacttgtggctcagacctgagtgcaaggtggaaataaacatcaaacatcttttcattatcccta

SEQ ID NO: 58 - DNA, 2439 bp, Homo sapiens
  source 1..2439: mol_type = other DNA
gagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagctgtatttccaaaatgtcgctttctaacaagctgacgctggacaagctggacgttaaagggaagcgggtcgttatgagagtcgacttcaatgttcctatgaagaacaaccagataacaaacaaccagaggattaaggctgctgtcccaagcatcaaattctgcttggacaatggagccaagtcggtagtccttatgagccacctaggccggcctgatggtgtgcccatgcctgacaagtactccttagagccagttgctgtagaactcaaatctctgctgggcaaggatgttctgttcttgaaggactgtgtaggcccagaagtggagaaagcctgtgccaacccagctgctgggtctgtcatcctgctggagaacctccgctttcatgtggaggaagaagggaagggaaaagatgcttctgggaacaaggttaaagccgagccagccaaaatagaagctttccgagcttcactttccaagctaggggatgtctatgtcaatgatgcttttggcactgctcacagagcccacagctccatggtaggagtcaatctgccacagaaggctggtgggtttttgatgaagaaggagctgaactactttgcaaaggccttggagagcccagagcgacccttcctggccatcctgggcggagctaaagttgcagacaagatccagctcatcaataatatgctggacaaagtcaatgagatgattattggtggtggaatggcttttaccttccttaaggtgctcaacaacatggagattggcacttctctgtttgatgaagagggagccaagattgtcaaagacctaatgtccaaagctgagaagaatggtgtgaagattaccttgcctgttgactttgtcactgctgacaagtttgatgagaatgccaagactggccaagccactgtggcttctggcatacctgctggctggatgggcttggactgtggtcctgaaagcagcaagaagtatgctgaggctgtcactcgggctaagcagattgtgtggaatggtcctgtgggggtatttgaatgggaagcttttgcccggggaaccaaagctctcatggatgaggtggtgaaagccacttctaggggctgcatcaccatcataggtggtggagacactgccacttgctgtgccaaatggaacacggaggataaagtcagccatgtgagcactgggggtggtgccagtttggagctcctggaaggtaaagtccttcctggggtggatgctctcagcaatatttagtactttcctgccttttagttcctgtgcacagcccctaagtcaacttagcattttctgcatctccacttggcattagctaaaaccttccatgtcaagattcagctagtggccaagagatgcagtgccaggaacccttaaacagttgcacagcatctcagctcatcttcactgcaccctggatttgcatacattcttcaagatcccatttgaattttttagtgactaaaccattgtgcattctagagtgcatatatttatattttgcctgttaaaaagaaagtgagcagtgttagcttagttctcttttgatgtaggttattatgattagctttgtcactgtttcactactcagcatggaaacaagatgaaattccatttgtaggtagtgagacaaaattgatgatccattaagtaaacaataaaagtgtccattgaaaccgtgatttttttttttttcctgtcatactttgttaggaagggtgagaatagaatcttgaggaacggatcagatgtctatattgctgaatgcaagaagtggggcagcagcagtggagagatgggacaattagataaatgtccattctttatcaagggcctactttatggcagacattgtgctagtgcttttattctaacttttatttttatcagttacacatgatcataatttaaaaagtcaaggcttataacaaaaaagccccagcccattcctcccattcaagattcccactccccagaggtgaccactttcaactcttgagtttttcaggtatatacctccatgtttctaagtaatatgcttatattgttcacttcttttttttttattttttaaagaaatctatttcataccatggaggaaggctctgttccacatatatttccacttcttcattctctcggtatagttttgtcacaattatagattagatcaaaagtctacataactaatacagctgagctatgtagtatgctatgattaaatttacttatgtaaaaaaaaaaaaaaaaaa

SEQ ID NO: 59 - DNA, 1196 bp, Homo sapiens
  source 1..1196: mol_type = other DNA
cacttctgccgcccctgtttcaagggataagaaaccctgcgacaaaacctcctccttttccaagcggctgccgaagatggcggaggtgcaggtcctggtgcttgatggtcgaggccatctcctgggccgcctggcggccatcgtggctaaacaggtactgctgggccggaaggtggtggtcgtacgctgtgaaggcatcaacatttctggcaatttctacagaaacaagttgaagtacctggctttcctccgcaagcggatgaacaccaacccttcccgaggcccctaccacttccgggcccccagccgcatcttctggcggaccgtgcgaggtatgctgccccacaaaaccaagcgaggccaggccgctctggaccgtctcaaggtgtttgacggcatcccaccgccctacgacaagaaaaagcggatggtggttcctgctgccctcaaggtcgtgcgtctgaagcctacaagaaagtttgcctatctggggcgcctggctcacgaggttggctggaagtaccaggcagtgacagccaccctggaggagaagaggaaagagaaagccaagatccactaccggaagaagaaacagctcatgaggctacggaaacaggccgagaagaacgtggagaagaaaattgacaaatacacagaggtcctcaagacccacggactcctggtctgagcccaataaagactgttaattcctcatgcgttgcctgcccttcctccattgttgccctggaatgtacgggacccaggggcagcagcagtccaggtgccacaggcagccctgggacataggaagctgggagcaaggaaagggtcttagtcactgcctcccgaagttgcttgaaagcactcggagaattgtgcaggtgtcatttatctatgaccaataggaagagcaaccagttactatgagtgaaagggagccagaagactgattggagggccctatcttgtgagtggggcatctgttggactttccacctggtcatatactctgcagctgttagaatgtgcaagcacttggggacagcatgagcttgctgttgtacacagggtatttctagaagcagaaatagactgggaagatgcacaaccaaggggttacaggcatcgcccatgctcctcacctgtattttgtaatcagaaataaattgcttttaaagaaaaaaaaaaaaaaaaaa

SEQ ID NO: 60 - DNA, 987 bp, Homo sapiens
  source 1..987: mol_type = other DNA
aatataagtggaggcgtcgcgctggcgggcattcctgaagctgacagcattcgggccgagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaagcagcatcatggaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttgatgtgttatctctta

SEQ ID NO: 61 - DNA, 3003 bp, Homo sapiens
  source 1..3003: mol_type = other DNA
ctttctccttccccttcttccgggctcccgtcccggctcatcacccggcctgtggcccactcccaccgccagctggaaccctggggactacgacgtccctcaaaccttgcttctaggagataaaaagaacatccagtcatggataaaaatgagctggttcagaaggccaaactggccgagcaggctgagcgatatgatgacatggcagcctgcatgaagtctgtaactgagcaaggagctgaattatccaatgaggagaggaatcttctctcagttgcttataaaaatgttgtaggagcccgtaggtcatcttggagggtcgtctcaagtattgaacaaaagacggaaggtgctgagaaaaaacagcagatggctcgagaatacagagagaaaattgagacggagctaagagatatctgcaatgatgtactgtctcttttggaaaagttcttgatccccaatgcttcacaagcagagagcaaagtcttctatttgaaaatgaaaggagattactaccgttacttggctgaggttgccgctggtgatgacaagaaagggattgtcgatcagtcacaacaagcataccaagaagcttttgaaatcagcaaaaaggaaatgcaaccaacacatcctatcagactgggtctggcccttaacttctctgtgttctattatgagattctgaactccccagagaaagcctgctctcttgcaaagacagcttttgatgaagccattgctgaacttgatacattaagtgaagagtcatacaaagacagcacgctaataatgcaattactgagagacaacttgacattgtggacatcggatacccaaggagacgaagctgaagcaggagaaggaggggaaaattaaccggccttccaacttttgtctgcctcattctaaaatttacacagtagaccatttgtcatccatgctgtcccacaaatagttttttgtttacgatttatgacaggtttatgttacttctatttgaatttctatatttcccatgtggtttttatgtttaatattaggggagtagagccagttaacatttagggagttatctgttttcatcttgaggtggccaatatggggatgtggaatttttatacaagttataagtgtttggcatagtacttttggtacattgtggcttcaaaagggccagtgtaaaactgcttccatgtctaagcaaagaaaactgcctacatactggtttgtcctggcggggaataaaagggatcattggttccagtcacaggtgtagtaattgtgggtactttaaggtttggagcacttacaaggctgtggtagaatcataccccatggataccacatattaaaccatgtatatctgtggaatactcaatgtgtacacctttgactacagctgcagaagtgttcctttagacaaagttgtgacccattttactctggataagggcagaaacggttcacattccattatttgtaaagttacctgctgttagctttcattatttttgctacactcattttatttgtatttaaatgttttaggcaacctaagaacaaatgtaaaagtaaagatgcaggaaaaatgaattgcttggtattcattacttcatgtatatcaagcacagcagtaaaacaaaaacccatgtatttaacttttttttaggatttttgcttttgtgatttttttttttttgatacttgcctaacatgcatgtgctgtaaaaatagttaacagggaaataacttgagatgatggctagctttgtttaatgtcttatgaaattttcatgaacaatccaagcataattgttaagaacacgtgtattaaattcatgtaagtggaataaaagttttatgaatggacttttcaactactttctctacagcttttcatgtaaattagtcttggttctgaaacttctctaaaggaaattgtacattttttgaaatttattccttattccctcttggcagctaatgggctcttaccaagtttaaacacaaaatttatcataacaaaaatactactaatataactactgtttccatgtcccatgatcccctctcttcctccccaccctgaaaaaaatgagttcctattttttctgggagagggggggattgattagaaaaaaatgtagtgtgttccatttaaaattttggcatatggcattttctaacttaggaagccacaatgttcttggcccatcatgacattgggtagcattaactgtaagttttgtgcttccaaatcactttttggtttttaagaatttcttgatactcttatagcctgccttcaattttgatcctttattctttctatttgtcaggtgcacaagattaccttcctgttttagccttctgtcttgtcaccaaccattcttacttggtggccatgtacttggaaaaaggccgcatgatctttctggctccactcagtgtctaaggcaccctgcttcctttgcttgcatcccacagactatttccctcatcctatttactgcagcaaatctctccttagttgatgagactgtgtttatctccctttaaaaccctacctatcctgaatggtctgtcattgtctgcctttaaaatccttcctctttcttcctcctctattctctaaataatgatggggctaagttatacccaaagctcactttacaaaatatttcctcagtactttgcagaaaacaccaaacaaaaatgccattttaaaaaaggtgtattttttcttttagaatgtaagctcctcaagagcagggacaatgttttctgtatgttctattgtgcctagtacactgtaaatgctcaataaatattgatgatgggaggcagtgagtcttgatgataagggtgagaaactgaaatcccaaacactgttttgttgcttgttttattatgacctcagattaaattgggaaatattggcccttttgaataattgtcccaaatattacattcaaataaaagtgcaatggagaaaaaaaaaaa

SEQ ID NO: 62 - DNA, 2803 bp, Homo sapiens
  source 1..2803: mol_type = other DNA
actgcagccccgctcgactccggcgtggtgcgcaggcgcggtatcccccctcccccgccagctcgaccccggtgtggtgcgcaggcgcagtctgcgcagggactggcgggactgcgcggcggcaacagcagacatgtcgggggtccggggcctgtcgcggctgctgagcgctcggcgcctggcgctggccaaggcgtggccaacagtgttgcaaacaggaacccgaggttttcacttcactgttgatgggaacaagagggcatctgctaaagtttcagattccatttctgctcagtatccagtagtggatcatgaatttgatgcagtggtggtaggcgctggaggggcaggcttgcgagctgcatttggcctttctgaggcagggtttaatacagcatgtgttaccaagctgtttcctaccaggtcacacactgttgcagcacagggaggaatcaatgctgctctggggaacatggaggaggacaactggaggtggcatttctacgacaccgtgaagggctccgactggctgggggaccaggatgccatccactacatgacggagcaggcccccgccgccgtggtcgagctagaaaattatggcatgccgtttagcagaactgaagatgggaagatttatcagcgtgcatttggtggacagagcctcaagtttggaaagggcgggcaggcccatcggtgctgctgtgtggctgatcggactggccactcgctattgcacaccttatatggaaggtctctgcgatatgataccagctattttgtggagtattttgccttggatctcctgatggagaatggggagtgccgtggtgtcatcgcactgtgcatagaggacgggtccatccatcgcataagagcaaagaacactgttgttgccacaggaggctacgggcgcacctacttcagctgcacgtctgcccacaccagcactggcgacggcacggccatgatcaccagggcaggccttccttgccaggacctagagtttgttcagttccaccctacaggcatatatggtgctggttgtctcattacggaaggatgtcgtggagagggaggcattctcattaacagtcaaggcgaaaggtttatggagcgatacgcccctgtcgcgaaggacctggcgtctagagatgtggtgtctcggtccatgactctggagatccgagaaggaagaggctgtggccctgagaaagatcacgtctacctgcagctgcaccacctacctccagagcagctggccacgcgcctgcctggcatttcagagacagccatgatcttcgctggcgtggacgtcacgaaggagccgatccctgtcctccccaccgtgcattataacatgggcggcattcccaccaactacaaggggcaggtcctgaggcacgtgaatggccaggatcagattgtgcccggcctgtacgcctgtggggaggccgcctgtgcctcggtacatggtgccaaccgcctcggggcaaactcgctcttggacctggttgtctttggtcgggcatgtgccctgagcatcgaagagtcatgcaggcctggagataaagtccctccaattaaaccaaacgctggggaagaatctgtcatgaatcttgacaaattgagatttgctgatggaagcataagaacatcggaactgcgactcagcatgcagaagtcaatgcaaaatcatgctgccgtgttccgtgtgggaagcgtgttgcaagaaggttgtgggaaaatcagcaagctctatggagacctaaagcacctgaagacgttcgaccggggaatggtctggaacacggacctggtggagaccctggagctgcagaacctgatgctgtgtgcgctgcagaccatctacggagcagaggcacggaaggagtcacggggcgcgcatgccagggaagactacaaggtgcggattgatgagtacgattactccaagcccatccaggggcaacagaagaagccctttgaggagcactggaggaagcacaccctgtcctatgtggacgttggcactgggaaggtcactctggaatatagacccgtgatcgacaaaactttgaacgaggctgactgtgccaccgtcccgccagccattcgctcctactgatgagacaagatgtggtgatgacagaatcagcttttgtaattatgtataatagctcatgcatgtgtccatgtcataactgtcttcatacgcttctgcactctggggaagaaggagtacattgaagggagattggcacctagtggctgggagcttgccaggaacccagtggccagggagcgtggcacttacctttgtcccttgcttcattcttgtgagatgataaaactgggcacagctcttaaataaaatataaatgaacaaactttcttttatttccaaatccatttgaaatattttactgttgtgactttagtcatatttgttgacctaaaaatcaaatgtaatctttgtattgtgttacatcaaaatccagatattttgtatagtttcttttttctttttcttttcttttttttttttgagacaggatcggtgcagtagtacaatcacagctcactgcagcctcaaactcctgggcagctcaggtgatcttcctgactcagccttctgagtagttggggctacaggtgtgcaccaccatgcccagctcatttattttgtaattgtagggacagggtctcactgtgttgcctaggctggtctcaagtgatcctccctccttggcctcccaaggtgctggaattataggtgtgaacaaaccaaaaaaaaaaaaaa

SEQ ID NO: 63 - DNA, 1435 bp, Homo sapiens
  source 1..1435: mol_type = other DNA
ggcggggcctgcttctcctcagcttcaggcggctgcgacgagccctcaggcgaacctctcggctttcccgcgcggcgccgcctcttgctgcgcctccgcctcctcctctgctccgccaccggcttcctcctcctgagcagtcagcccgcgcgccggccggctccgttatggcgacccgcagccctggcgtcgtgattagtgatgatgaaccaggttatgaccttgatttattttgcatacctaatcattatgctgaggatttggaaagggtgtttattcctcatggactaattatggacaggactgaacgtcttgctcgagatgtgatgaaggagatgggaggccatcacattgtagccctctgtgtgctcaaggggggctataaattctttgctgacctgctggattacatcaaagcactgaatagaaatagtgatagatccattcctatgactgtagattttatcagactgaagagctattgtaatgaccagtcaacaggggacataaaagtaattggtggagatgatctctcaactttaactggaaagaatgtcttgattgtggaagatataattgacactggcaaaacaatgcagactttgctttccttggtcaggcagtataatccaaagatggtcaaggtcgcaagcttgctggtgaaaaggaccccacgaagtgttggatataagccagactttgttggatttgaaattccagacaagtttgttgtaggatatgcccttgactataatgaatacttcagggatttgaatcatgtttgtgtcattagtgaaactggaaaagcaaaatacaaagcctaagatgagagttcaagttgagtttggaaacatctggagtcctattgacatcgccagtaaaattatcaatgttctagttctgtggccatctgcttagtagagctttttgcatgtatcttctaagaattttatctgttttgtactttagaaatgtcagttgctgcattcctaaactgtttatttgcactatgagcctatagactatcagttccctttgggcggattgttgtttaacttgtaaatgaaaaaattctcttaaaccacagcactattgagtgaaacattgaactcatatctgtaagaaataaagagaagatatattagttttttaattggtattttaatttttatatatgcaggaaagaatagaagtgattgaatattgttaattataccaccgtgtgttagaaaagtaagaagcagtcaattttcacatcaaagacagcatctaagaagttttgttctgtcctggaattattttagtagtgtttcagtaatgttgactgtattttccaacttgttcaaattattaccagtgaatctttgtcagcagttcccttttaaatgcaaatcaataaattcccaaaaatttaaaaaaaaaaaaaaaaaaaaaa

SEQ ID NO: 64 - DNA, 4476 bp, Homo sapiens
  source 1..4476: mol_type = other DNA
agcagagagaacacacgtccttgcggaagtgacggcagttccgagtccagtgggggcggtgggagcgatgagggtctgagacggtgggagcggttgtgtgaagatggagacattccatacaccaagcttgggtgatgaggaatttgaaatcccacctatctccttggattctgatccctcattggctgtctcagatgtggttggccactttgatgacctggcagacccttcctcttcacaggatggcagtttttcagcccagtatggggtccagacattggacatgcctgtgggcatgacccatggcttgatggagcagggcggggggctcctgagtgggggcttgaccatggacttggaccactctataggaactcagtatagtgccaacccacctgttacaattgatgtaccaatgacagacatgacatctggcttgatggggcatagccagttgaccaccattgatcagtcagaactgagttcccagctgggtttgagcctagggggtggcaccatcctgccacctgcccagtcacctgaagatcgtctttcaaccaccccttcacctactagttcacttcacgaggatggtgttgaggatttccggaggcaacttcccagccagaagacagtcgtggtggaagcagggaaaaagcagaaggccccaaagaagagaaaaaagaaagatcctaatgaacctcagaaaccagtttcagcatatgctttattctttcgtgatacacaggctgccatcaagggacagaatcctaatgccacttttggtgaggtttcaaaaattgtggcctccatgtgggatagtcttggagaggagcaaaaacaggtatataagaggaaaactgaggctgccaagaaagagtatctgaaggcactggctgcttacaaagacaaccaggagtgtcaggccactgtggaaacagtggaattggatccagcaccaccatcacaaactccttctccacctcctatggctactgttgacccagcatctccagcaccagcttcaatagagccccctgccctgtccccatccattgttgttaactccaccctttcatcctatgtggcaaaccaggcatcttctggagctgggggtcagcccaatatcaccaagttgattattaccaaacaaatgttgccctcttctattactatgtctcaaggagggatggttactgttatcccagccacagtggtgacctcccgggggctccaactaggccaaaccagtacagctactatccagcccagtcaacaagcccagattgtcactcggtcagtgttgcaggcagcagcagctgctgctgctgctgcttctatgcaactgcctccaccccgactacagccccctccattacaacagatgccacagcccccgactcagcagcaagttaccattctgcagcagcctcctccactccaggccatgcaacagcctccacctcagaaagttcgaatcaatttacagcaacagcctcctcctctgcagatcaagagtgtgcctctacccactttgaaaatgcagactaccttagtcccaccaactgtggaaagtagtcctgagcggcctatgaacaacagccctgaggcccatacagtggaggcaccttctcctgagactatctgtgagatgatcacagatgtagttcctgaggttgagtctccttctcagatggatgttgaattggtgagtgggtctcctgtggcactctcaccccagcctcgatgtgtgaggtctggttgtgagaaccctcccattgtgagtaaggactgggacaatgaatactgcagcaatgagtgtgtggtgaagcactgcagggatgtattcttggcctgggtagcctctagaaattcaaacacagtggtgtttgtgaaatagtccttcctgttctccaagccagtgaagagttatctgctgggaaagtgtccaagagcctgtttttgaaacacaagctgggcttctggtagtgcctcatcacaacccatgatggctgttcatgtttcaccccttttcttccttcagcagaggccaggctatggagcagggccactgaatttgctgtaatctggagatgctttttactttcaaccataagcggtaatagcagaggaaagggtgaagggagtctgggcaagcaaagcatagagatggtggggtggtggtggggttgaagaaacttgttggtataattgtcataggacttgcctaaaatattattaaaattacgggagtgtactcagctttgagcctaggagaaaatgccactgtgtgcatccattttaaagggttccctcataaaaaaatgttattccccattatcacatcagtacactgctttgaaaacaaaacttttcaacatgggcatactgggctacatggaaaatgacatcacccaggagtgatttctctttatatatattatttctgcagttaccatccttatctgagttatcacagttcatgaatctaagaggcggaactctacatcattagtaagaggttccaccaaagtctaaagttgtattcacttgtgtttgatgaactatctttaaaagaccataggtctatcattatttcttagacataatctaaagaaaaacagactagagaagccacctggttgtaacagaataagcagaagtttacagcatgatagtccaagtggtgataactttaaataaaactcaaatttttactgtttgtagacaggaatgctgtcctagagaacctcctcctcaaccagctacgtacatagttttatcctatgcattcctgttttctgtgtgttttttgttttttttttttttttttttttttgagacagagtctcgctctgtcacccaggctggagtgcagtggtgcgacctcagctcactgaaacctctgcctcccgggttcaagcgattctcctgcatcagcctcccgagtagctaggattacaggcgcccgccactacgcccagctaatttgtggtatttttagtagagacagggtttcaccatgttggccaggctggtctcgaactcctgacctcatgatccgcccgccttgacctcccaaagtgctgggattacaggcatgagccaccgcacccagcctgcattcctgtttttttaatggttttggagggtagcagtagagatggggtctcactatgttgcccagtctagtcttgaactcctgggctacagttaccctcctacctcggcttcccaaagtgctcggattacaggtgtgagccactgtgcctagcctataatgatcattttaatgtttcccatgcactcatttagtttgaaccttcacagcaacccaatgaggtaatactcccatttcacatataatactgagagatgagttgcacaagattatacactgttaagtagcagagccagaatggacttcagaatcccaactacaatacaaatgtttatttaaataaagaagaaagctattgtacaaatatcactcttcaggtttagcttacagagccatggctatggattcttagctctgtaaggaagtgcttctataaattcttaggtttagagatgataccatctgggtacctttgcttgaaccgtgcaaccacatctgggtctagtaggtggatcccatccagttggtttccaagggtgatcctgaaacagtgtaaaaggaggggcaaaccagaaatcctggaattagagggtttaatattgttaaaaaatgcataccaaatgaagactgcctatcatcatatcaaatatgccaattctaaaaagagcttaacattagaatagtatatggtagaattactagttcagaattggcatagattctggtgttaaaatagactggatctgtattatctgagggttagtaactaatgcttagccaggcctgcttcacagagttgctaccagggagtattctttggataagcaaaatgctagcagcatgtgttttaagctctgttaaggggtgaaagatgtaattattgacagattaaatagataacttcgtaaccaccagggggcagattcaatacatcacagaatggctgaggaagatccttgggttgtgaagagagtagaaaccctagggagcagtgcttttgggtcctagaacctgttgagtttctaatgaatatttgtagaatctcataaaacagtttaaatacaagcttaagtggcttatgaatcctgtgaagctcatttatggactagtgtaaaacaatgtgaagctctactaagttctgtccttaatcataaataatagccccttgaggactagcctgttctctggtcaccttaccagttgggttgcacattgtgtggtcgtccaaataactcaatcttgcgagtgccaggagatagtctttcaatcatgccatagatttcatctggtttatgactggtggaacgaacctaggaaataaaaactagctgctttttaagttacacaagaaaaaa

SEQ ID NO: 65 - DNA, 4814 bp, Homo sapiens
  source 1..4814: mol_type = other DNA
cttcgtgccacgtcaccgcctgcgtcgcttccggaggcgcagcgggcgatgacgtagagggacgtgccctctatatgaggttggggagcggctgagtcggccttttccgcccgctcccccctccccccgagcgccgctccggctgcaccgcgctcgctccgagtttcaggctcgtgctaagctagcgccgtcgtcgtctcccttcagtcgccatcatgattatctaccgggacctcatcagccacgatgagatgttctccgacatctacaagatccgggagatcgcggacgggttgtgcctggaggtggaggggaagatggtcagtaggacagaaggtaacattgatgactcgctcattggtggaaatgcctccgctgaaggccccgagggcgaaggtaccgaaagcacagtaatcactggtgtcgatattgtcatgaaccatcacctgcaggaaacaagtttcacaaaagaagcctacaagaagtacatcaaagattacatgaaatcaatcaaagggaaacttgaagaacagagaccagaaagagtaaaaccttttatgacaggggctgcagaacaaatcaagcacatccttgctaatttcaaaaactaccagttctttattggtgaaaacatgaatccagatggcatggttgctctattggactaccgtgaggatggtgtgaccccatatatgattttctttaaggatggtttagaaatggaaaaatgtgatgcaaaagaaagaaatccctgcgctttctgtctgtctttgtggcggcccagattgaattggggaatacatctttagcctggaaatgtaggctgcatgttaatggtaatgtaacttttgcagtgtaatgtttgaaaaatattaatgtagtttttgcttttacagtaacaaatgtggcaattattttggatctatcacctgtcatcataactggcttctgcttgtcatccacacaacaccaggacttaagacaaatgggactgatgtcatcttgagctcttcatttattttgactgtgatttatttggagtggaggcattgtttttaagaaaaacatgtcatgtaggttgtctaaaaataaaatgcatttaaactcatttgagagaatgccttttagtttaatgcatatttaaactaaattgatcctgtagtgttcctggagaagctagagcctgattgtaggctactactcatcaattaacttctacagtggagactacttctgggactggaatataaaaaagaatcaaaggttctgattttgagttgcaataaagggaaagaccatgctcatagcagtgccaacatctgaagtgtggagccttacccatttcatcacctacaacggaagtagttaactggaagagattaccaagagaataaaaagagactcattcagtggaagcaactttgtctcagcttatttcacataaagagagcgaagtcttttgggatgaatgttaattaaactccctggtaactagaacagggactggcaaactagcctatctgaccacctgttttgtacactttaaggtggttggttgcctttttaaatggttgaggggaaaagaataccttgtgggatatggaatttaagttcgagtccagttttattggaacgtggctatgcttattcatttatggattgactgtggctgttgtcagtgcatgagcagagttgtgtctaacagactagagcctgcaagtttgccagcccctgatttaaaagatgaaggtacacagaatgtgggctggctggtgggcaaaggggtaaaaatgttctctatattgtatctgaaaagatggggtgtctgaataagaaaatgcatctatttgacagacctggagcagttgctatctgctgctatggtttccaccacagatgcaagaagaacatgtccttgcgctttccgtctgtctaattgtggcagctgagattgaatagaggaatacaggaggaaaaaaagcgggaagagtttttgaggcaggtcggtcacccaggcttgtagtgcagtggcacaagcaactcactgcattctctgcatcctgtgctcaagccattttcccacctcagtctcactagttgctgggactgcaggcatgcacccctatgcccagctaatttttgtagagaccgagtatcgcttagttgcccagggtggtctcaactcctgggctcaaggagatctgcccacctcagcctcccaaagtgcaggcctagcctgggaggggaattttcaaaacgtgagttttgggaaatagtctatcagccttacctggttgattacacttgtaaaagaaagattaaaagcaggccagtgactctggtctgcttgaacatgtgaatgtagtggtttgagcaatctggagtttgccctagtgtcaaattccagactgtccatagtgtccaaaacctgaggcagatactaatgttaacccccagcaccccgtgattggaaacaaacctaaatacgtattgggaacttaatagcaattttaagcattctgatagattttttgtagggatggggtcatgccatgtggcccaggctggtctgaaaactctggcctcaagtgatctcaagctttggccttctaaagtgttgggattacaggtgtgaggcattgcacctggcttagcgttctgatttgacattgtaatgaaaagtgtgagtctcatctacagggccttttgtcctctgaaatgatagcaggaagggaattttcaggcagtggtcaaagctggggaaaccaggatagtgaagaaggccttgaggtgagagatggaagctaattggtgaactagccttggaagcctgaaacagacaagtagcaattcagagactttgtgggctccactgctccaacttgttttgaagattttcagttctgcagaagaggtatttccccagttgtcctttcagtgctcttagctgttttcccaacatccagatccaatcaaggctgggacatagcattttatcatgtctatttaagtcagaagtgatgaaccccagctgtttacctcatggtaaacctttgaagattccaggtagaatcttctcagactttgaagactgtctcattttatatctttttctcgttattcctagggtcaagacgttttgggcaagaataaggatgtgaacatcagaaagctcataacattttgtttttgatgctaagtttaacaaaggcatgctttagtagcctgtgggccctagggtttgttaaagtgtggagaacaactgagtggagcaagaggacttttctaggaaggtccttgtaatgtgacatttgaaaacaaatgaaggtgtggaagtaggccatgtggatatcaggacaaaccattccaggccaagacaacagcagttagtctggagtgtgatgtgttctgggaaaaaagtggccactttgctaacccaagaagacaggaagggttgtaaagcagtgggagtgtgcaaggaaggaagaccagacctcaaggaaaccacaggcgctctgagcagaagagttacatgatatgactcaaatttttaaaggatcactttggctgccaggtggcagggtaaaagcatagaataattgtgtataatgtgtttttaaggcaaagatagtggcttagtctagggtagtagactgaggtggtaggaaatgaagatagagacaacaggatatgctggtgggtgaggatggatttaatgttgatacaagtattttggtctgagcgtttggaagaaagttggcactgaggtgggaagtcgagtttagttttgttagttttggatgtgttaagtttgagatgctgattcttcagagaagtctaagctggagaactatatagagagtggaaagataacaatagacattgaaagccatgatacaggataaggtcatttggagagaggatagactgcattccaacatgagattggttgacaaagagaaaccaacaaaggtaattaagaggtgctcccactgcacttgtactcagaaggctgaggtaggattgttagaggccagcctgggcaccacagggagaccccatctctaaaatttagccaggaaccatggctcatgcctgtagccccaggaatttgggaggctgagtggggaggatcgcttgaggtcaggagtttgagaccagcctgggcaacatagggagacctaaaaaaattaattgggcatctgtagtcccagctactcaggcggctgagctgagaggatggcttgagtccgagagattgagggtgcagtgagctgtgatcataccactgcactccagcctgggcggcagtgagacactatctgaaaaaagtttaaaaattttaaaaaagaaggaactgcccctgaggtaagaaccaagggagggcctcccagaggtcaggtggaaaaagttttaggaaggaggaagtagtcaacagggttacctgttgcaaagtacttaagtaatatgaggcctgatagtggtaaacttgactaccgttggatttcactagtgggaaaggaagtctaattaaaatgcactcaagagactaacagtcgcaggcatgaaatacaatacaggtacatggttttttattatgtgtgcatctgcttcagtaataggtgtgaattactcatttggatcattaggagtttcaaaatctagttaaatgactagatttttgttgatgtaaattctgtcattctgaactgcagggattgtcagtaacttaactgcaaactaaactggtgataattatggtaaaattgcaagacgagcaataaatctcaaccaacttgagagaacactgataa